SEQUENCE LISTING (14 sequences)

SEQ ID NO: 1 - protein, 26 aa, Homo Sapiens
HRGVNNSYIQRSEHPLAQLYCHSIME

SEQ ID NO: 2 - protein, 342 aa, Homo Sapiens
EEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 3 - protein, 874 aa, Homo Sapiens
ERAGPSFGQQRQQQQPQQQKQQQRDQDSVEAWLDDHWDFTFSYFVRKATREMVNAWFAERVHTIPVCKEGIRGHTESCSCPLQQSPRADNSVPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 4 - protein, 27 aa, Homo Sapiens
HPGVSNQFLINTNSELALMYNDESVLE

SEQ ID NO: 5 - protein, 326 aa, Homo Sapiens
EEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHPGVSNQFLINTNSELALMYNDESVLEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAE

SEQ ID NO: 6 - protein, 858 aa, Homo Sapiens
ERAGPSFGQQRQQQQPQQQKQQQRDQDSVEAWLDDHWDFTFSYFVRKATREMVNAWFAERVHTIPVCKEGIRGHTESCSCPLQQSPRADNSVPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHPGVSNQFLINTNSELALMYNDESVLEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAE

SEQ ID NO: 7 - DNA, 34 bp, Homo Sapiens
cgtgaattcatggaggaagaaacaagagagctac

SEQ ID NO: 8 - DNA, 42 bp, Homo Sapiens
cgttctagactatcagttccgcttggcctggccgctttcccc

SEQ ID NO: 9 - DNA, 79 bp, Homo Sapiens
cgtgaattcatgcatcatcatcatcatcatcttctggttccgcgtggatctgcgcccgaggaagaaacaagagagctac

SEQ ID NO: 10 - DNA, 72 bp, Homo Sapiens
caaagaaagttctgaatttgtgttgatgagaaactgattggagactccaggatgatccaaatcgtggcttag

SEQ ID NO: 11 - DNA, 72 bp, Homo Sapiens
atcaacacaaattcagaacttgctttgatgtataatgatgaatctgtgttggaacaccatcattttgaccag

SEQ ID NO: 12 - DNA, 39 bp, Homo Sapiens
cgttctagactatcattctgcaagggcctgccatttctg

SEQ ID NO: 13 - DNA, 31 bp, Homo Sapiens
cgtcatatggaggaagaaacaagagagctac

SEQ ID NO: 14 - DNA, 39 bp, Homo Sapiens
cgtctcgagctatcattctgcaagggcctgccatttctg

(19) United States
(12) Patent Application Publication
(10) Pub. No.: US 2004/0082052 A1
(43) Pub. Date: Apr. 29, 2004

(54) Crystal structure

(75) Inventors: David Graham Brown, Sandwich (GB); Colin Roger Groom, Sandwich (GB); Andrew Lee Hopkins, Sandwich (GB); Timothy Mark Jenkins, Sandwich (GB); Sarah Helen Kamp, Sandwich (GB); Margaret Mary O'Gara, Sandwich (GB); Heather Joan Ringrose, Sandwich (GB); Colin Mark Robinson, Sandwich (GB); Wendy Elaine Taylor, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/427,222

(22) Filed: May 1, 2003

(30) Foreign Application Priority Data
Oct. 24, 2002 (IB) PC/IB02/04426

(51) Int. Cl. G06F 19/00; G01N 33/48; G01N 33/50; C12N 9/16

(52) U.S. Cl. 435/196; 702/19

(57) ABSTRACT

The present invention relates to the soakable crystal structures of a phosphodiesterase 5 (PDE5) and their uses in identifying PDE5 ligands, including PDE5 inhibitor compounds. The present invention also relates to methods of identifying such PDE5 inhibitor compounds and their medical use. The present invention additionally relates to crystals of PDE5 into which ligands may be soaked and crystals of PDE5 comprising PDE5 ligands that have been soaked into the crystal.

### RELATED APPLICATIONS AND PRIORITY CLAIMS THERETO

This application is a continuation-in-part of U.S. application No. (Not yet determined), filed on Apr. 30, 2003, entitled “Crystal Structure” of Brown et. al. which is the National Stage Application of International Application No. PC/IB02/04426 filed on Oct. 24, 2002, having designated the United States and published on (Not yet determined). The subject matter of the co-pending U.S. application No. (Not yet identified) and International Application No. GB/IB02/04426 is incorporated-by-reference in their entirety herein.

This application claims priority under 35 USC §119, 120 and 365 to the following foreign patent applications: Application No. GB 0126417.5 filed in the United Kingdom on Nov. 2, 2001, and International Application No. PC/IB02/04426 filed in the WO on Oct. 24, 2002 and having designated the U.S. as a receiving country.

### TECHNICAL FIELD

The present invention relates to crystal structures of a phosphodiesterase 5 (PDE5) and ligand complexes of PDE5 and to their uses in identifying PDE5 ligands, including PDE5 inhibitor compounds. The present invention also relates to methods of identifying such PDE5 inhibitor compounds and their medical use. The present invention additionally relates to crystals of PDE5 into which ligands may be soaked and to crystals of PDE5 comprising ligands that have been soaked into the crystal. Also contemplated by the present invention is the use of crystals of PDE5 into which ligands may be soaked in identifying ligands of PDE5, including PDE5 inhibitor compounds.

### BACKGROUND OF THE INVENTION

A wide variety of biological processes, including cardiac muscle contraction, regulation of blood flow, neural transmission, glandular secretion, cell differentiation and gene expression are affected by steady state levels of the cyclic nucleotide biological second messengers cAMP and cGMP. Intracellular receptors for these molecules include cyclic nucleotide dependent protein kinases (PGK) (Lohmann et al. 1997), cyclic nucleotide-gated channels, and class I phosphodiesterases (PDEs) (Charbonneau 1990). PDEs are a large family of proteins, which were first reported by Sutherland and co-workers (Rall & Sutherland 1958, Butcher & Sutherland 1962). The family of cyclic nucleotide phosphodiesterases catalyse the hydrolysis of 3′, 5′-cyclic nucleotides to the corresponding 5′ monophosphates. Current literature shows that there are eleven related, but biochemically distinct, human phosphodiesterase gene groups and that many of these groups include more than one gene subtype giving a total of twenty genes. Some PDEs are highly specific for hydrolysis of cAMP (PDE4, PDE7, PDE8), some are highly cGMP specific (PDE5, PDE6, PDE9), and some have mixed specificity (PDE1, PDE2, PDE3, PDE10, PDE11). All PDEs are multi-domain proteins; each PDE has a˜270 amino acid domain located towards the C-terminus, which has a high degree of amino acid sequence conservation between families (Charbonneau 1986). This domain has been extensively studied and shown to be responsible for the common catalytic function (Francis, S. H. et al. 1994). Non-homologous segments in the remainder of the protein have regulatory function or confer specific binding properties. PDE2, PDE5, PDE6 and PDE10 are all reported to contain putative GAF domains within their regulatory amino terminal portion (Aravind & Ponting 1997 and Soderling & Beavo 2000). These GAF domains have been shown to bind cGMP but their function is not yet fully understood. Full length mammalian PDEs characterised to date are dimeric in solution, but the relevance of the dimeric structure is unknown. The structure of the regulatory segment of PDE2A bound to cGMP has recently been solved and reveals a parallel dimer of four GAF domains, with cGMP binding to only one of the two GAF domains on each monomer (Martinez, et al. 2001).

PDE5, a cGMP specific PDE, has been recognised in recent years as an important therapeutic target. It is composed of the conserved C-terminal, zinc containing, catalytic domain, which catalyses the cleavage of cGMP, and an N-terminal regulatory portion, which contains two GAF domain repeats. Each GAF domain contains a cGMP-binding site, one of high affinity and the other of lower affinity. PDE5 activity is regulated through binding of cGMP to the high and low affinity cGMP binding sites followed by phosphorylation, which occurs only when both sites are occupied (Thomas et al. 1990). PDE5 is found in varying concentrations in a number of tissues including platelets, vascular and visceral smooth muscle, and skeletal muscle. The protein is a key regulator of cGMP levels in the smooth muscle of the erectile corpus cavernosal tissue. The physiological mechanism of erection involves release of nitric oxide (NO) in the corpus cavernosum during sexual stimulation. NO then activates the enzyme guanylate cyclase, which results in increased levels of cGMP, producing smooth muscle relaxation in the corpus cavernosum and allowing in flow of blood. Inhibition of PDE5 inhibits the breakdown of cGMP allowing the levels of cGMP, and hence smooth muscle relaxation, to be maintained (Corbin & Francis 1999). Sildenafil (UK-092,480), the active ingredient of Viagra® and a potent inhibitor of PDE5, has attracted widespread attention for the effective treatment of male erectile dysfunction.

Structural information has recently been shown for the catalytic domain of PDE4b a cAMP-specific PDE (Xu et al. 2000). This structure provides information about the overall fold of the catalytic domains of the PDE family, but, to date, no structural information is known about the way in which potential inhibitors bind to the enzyme.

The X-ray structure of a recombinant PDE5 comprising the catalytic domain in complex with Sildenafil has been determined (see earlier related patent application Number PCT/EB02/04426: Example 9, “crystallisation of wild type PDE5 catalytic domain with Sildenafil”, page 44, line 29 to page 45, line 17; Example 13, “Data collection, structure determination and refinement of wild type PDE5 with Sildenafil”, page 48, line 1 to page 49, line 9; Table 4, page 91 to 248; incorporated herein by reference). An engineered form of this PDE5, PDE5*, which shows improved qualities for the production of crystals of ligand complexes, has also been produced and the structure of such a complex has been determined (see Application Number PCT/IB02/04426: Example 11, “crystallisation of PDE5* with Sildenafil”, page 46, lines 8 to 25; Example 15, “data collection, structure determination and refinement of PDE5* with Sildenafil”, page 50, line 9 to page 51, line 6; and Table 6, “atomic coordinates for baculovirus-expressed PDE5* complex with Sildenafil”, pages 328 to 408; incorporated herein by reference). These complexes not only provide important structural information about this novel family of proteins but also assist the design of more potent and specific inhibitors of PDEs to treat the many diseases where PDEs play a role.

It is desirable to be able to obtain crystals of biological macromolecules in a complex with their ligands in order to obtain details of specific ligand interactions at a level of molecular resolution and, preferably, at atomic resolution. This invariably forms the critical step in providing information from which further, and possibly improved, ligands of the macromolecule may be designed and subsequently tested in further rounds of crystallisation. It is therefore most desirable that this process of obtaining complexes of the macromolecule be rapid and reproducible and minimise the need to constantly find new crystallisation conditions for each potential complex or use involved and time intensive structure solution methods for the solution of each potential complex structure. To this end the most desirable crystal form of a macromolecule of interest is one which is amenable to having potential ligands soaked into the preformed crystal of the apo macromolecule. This has distinct advantages over conventionally produced co-crystals. In particular no new crystallisation conditions need to be found for each potential complex. The lattice symmetry and cell dimensions for each new ligand complex will be substantially the same as for the apo macromolecule such that the data collection parameters for the apo and all complex crystals will essentially be identical. The structure solution can then occur using rapid difference fourier methods, thus avoiding more involved time and labour intensive phasing methods. Consequently, soakable crystals of macromolecules allow for particularly rapid screening and structural determination of new macromolecule-ligand complexes.

The engineered form of PDE5, PDE5*, allows the production of soakable crystals of ligand complexes, and the structure of such a soaked PDE5*-ligand complex has been determined.

### SUMMARY OF THE INVENTION

It has been found that PDE5 can be crystallised. It has also been found that manipulating the wild-type PDE5 amino acid sequence can facilitate the crystallisation of PDE5. Specifically, it has been found that manipulations of certain portions of the PDE5 amino acid sequence can facilitate the crystallisation of PDE5. More specifically it has been shown that particular manipulations of the PDE5 amino acid sequence can provide soakable crystal forms of PDE5 into which potential PDE5 ligands may be introduced by the process of crystal soaking.

It has been shown that manipulations of the catalytic domain of PDE5, specifically the 657-682 region of PDE5 (the “loop region”), can facilitate the crystallisation of PDE5 and particularly provide soakable crystals of PDE5. More specifically, manipulations of the loop region amino acid sequence (HRGVNNSYIQRSEHPLAQLYCHSIME=SEQ ID NO: 1) of PDE5 can facilitate the crystallisation of PDE5 and provide soakable crystals of PDE5. This manipulation can be achieved by deletion, addition or substitution of one or more amino acid residues of the PDE5 loop region or it can be achieved by complete replacement of the PDE5 loop region with a loop region (or other amino acid sequence) from another protein, preferably another PDE, more preferably PDE4, most preferably PDE4b.

Crystals of PDE5 have been found to be useful for screening for PDE5 ligands, especially PDE5 inhibitors by (i) co-crystallising PDE5 with the PDE5 ligand (e.g. PDE5 inhibitor), as shown in Application Number PCT/IB02/04426 (Example 9, “crystallisation of wild type PDE5 catalytic domain with Sildenafil”, page 44, line 29 to page 45, line 17; Example 11, “crystallisation of PDE5* with Sildenafil”, page 46, lines 8 to 25; Example 13, “Data collection, structure determination and refinement of wild type PDE5 with Sildenafil”, page 48, line 1 to page 49, line 9; Example 15, “data collection, structure determination and refinement of PDE5* with Sildenafil”, page 50, line 9 to page 51, line 6; Table 4 “atomic co-ordinates for wild type PDE5 complexed with Sildenafil”, pages 91 to 240; and Table 6, “atomic co-ordinates for baculovirus—expressed PDE5* complex with Sildenafil”, pages 328 to 408; incorporated herein by reference), or (ii) by soaking the PDE5 ligand (e.g. PDE5 inhibitor) into the crystal of PDE5, as shown in the present invention.

PDE5 ligands, especially PDE5 inhibitors, as identified by the methods of the present invention are useful in curative, palliative or prophylactic treatments.

Thus, the present invention provides the following (numbered) aspects:

### ASPECTS

1. A crystal of phosphodiesterase 5 (PDE5), wherein the crystal is soakable.

2. The crystal of PDE5 according to aspect 1, wherein said PDE5 is from a mammal.

3. The crystal of PDE5 according to aspect 1 or aspect 2, wherein said PDE5 is from a human.

4. The crystal of PDE5 according to any one of aspects 1 to 3, wherein said PDE5 is an isoform selected from the group consisting of PDE5A1, PDE5A2, PDE5A3 and PDE5A4.

5. The crystal of PDE5 according to any one of aspects 1 to 4, wherein said PDE5 comprises SEQ ID NO: 1 (PDE5 loop region) or a homologue, fragment, variant, analogue or derivative thereof.

SEQ ID NO: 1 is the so-called “loop region” of PDE5. This loop region or a homologue, fragment, variant, analogue or derivative thereof includes additions, deletions or substitutions of amino acid residues comprised within the loop region.

Preferably, a variant in relation to the amino acid sequence of the crystal of the PDE5 of the present invention includes the deletion or substitution of the histidine (His/H) residue as shown emboldened and underlined in SEQ ID NO: 1 (HRGVNNSYIQRSEHPLAQLYCHSIME). Replacement of said histidine (H) residue is preferably by way of incorporating one or more amino acid residues (other than histidine), preferably wherein said amino acid residues are neutral or non-polar.

More preferably, a variant in relation to the amino acid sequence of the crystal of the PDE5 of the present invention includes the complete replacement of the loop region with a loop region (or other amino acid sequence) from another protein, preferably a PDE, more preferably PDE4, most preferably PDE4b (see hereinafter).

6. The crystal of PDE5 according to any one of aspects 1 to 5, wherein said PDE5 comprises SEQ ID NO: 4 (PDE4 loop) or a homologue, fragment, variant, analogue or derivative thereof.

7. The crystal of PDE5 according to any one of aspects 1 to 6, wherein said PDE5 comprises SEQ ID NO: 5 (PDE5*=loop swapped PDE5 catalytic domain) or a homologue, fragment, variant, analogue or derivative thereof

8. The crystal of PDE5 according to any one of aspects 1 to 7, wherein said PDE5 comprises SEQ ID NO: 6 (full PDE5 sequence comprising PDE5*) or a homologue, fragment, variant, analogue or derivative thereof.

9. The crystal of PDE5 according to any of the preceding aspects which is grown using polyethylene glycol as a precipitant. Polyethylene glycol of molecular weights 2000 to 8000 are commonly used. Preferably the molecular weight of the polyethylene glycol is 4000. The polyethylene glycol may be at a concentration of between 10% and 30%, but is most preferably 20%. Additionally, the PDE5 protein used to grow the crystals is desired to be of a concentration between 1 and 20 mg/ml, preferably between 5 and 15 mg/ml and most preferably is at 10 mg/ml concentration.

10. The crystal of PDE5 according to any of the preceding aspects which is grown in a buffer in the pH range of 6.5 to 8.0. Preferably the pH is in the range of 7.0 to 7.8; most preferably the pH is 7.4. The buffer should be one capable of providing buffer capacity over the required pH range, preferably HEPES.

11. The crystal of PDE5 according to any of the preceding aspects which is grown in the presence of an alcohol. Preferably the alcohol is isopropanol.

12. The crystal of PDE5 according to any of the preceding aspects which is grown in a solution containing HEPES buffer, polyethylene glycol 4000 and iso-propanol. Preferably the solution contains 0.1M sodium HEPES pH 7.4, 20% polyethylene glycol 4000 and 10% iso-propanol. The crystal is grown from solution inclubated at or below room temperature (20-25° C.). Preferably the crystal is grown from a solution inclubated at a temperature within the range of 2-6° C. More preferably, the crystal is grown from a solution inclubated at about 4° C.

Soakable crystals may be grown using a variety of methods such as dialysis, sitting drop vapour diffusion or batch methods, microcrystallisation methods, micro or macro seeding methods or gel crystallisation methods but are preferably grown using hanging drop vapour diffusion.

13. The crystal of PDE5 as defined in any of the preceding aspects, which has one or more of the following characteristics:

(a) a space group C2;

(b) unit cell dimensions a˜56 Å±1%, b˜77 Å±1%, c˜81Å±1%, α=y=90°, β=103°±1%;

(c) 1 molecule per asymmetric unit;

(d) comprises a PDE5 of a molecular weight of approximately 40 kDa±2 kDa;

(e) a calculated solvent content of approximately 47±5%; and

(f) a monoclinic crystal system.

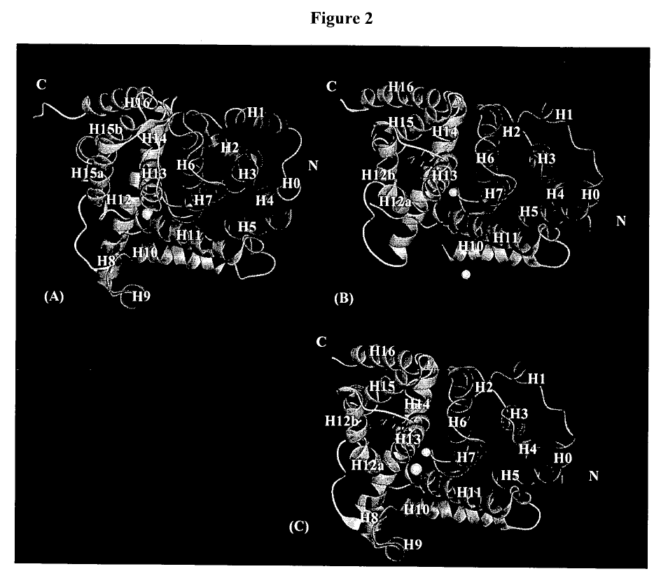

14. The crystal of PDE5 according to any of the preceding aspects, wherein PDE5 has an active site within the third sub-domain of the protein which is bounded by Helices 15 (H15 813-824) and 14 (H14 772-797), the C-terminus of Helix 13 (H13 749-765), and the C-terminus of Helix 11 (H11 706-721) along with the loop region between Helices 11 and 12a (H12a 725-731) as shown in FIG. 2.

15. The crystal of PDE5 according to aspect 14, wherein the active site is capable of accommodating a pyrazolo-pyrimidinone. An example of a preferred pyrazolo-pyrimidinone is UK-088,800. Another example of a pyrazolo-pyrimidinone is Sildenafil.

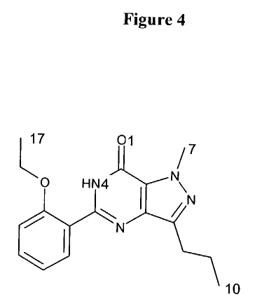

UK-088,800 is illustrated in FIG. 4 and is a structural representation of the compound with the chemical name 5-(2-ethoxyphenyl)-1-methyl-3-propyl-1,6-dihydro-7H-pyrazolo[4,3-d]pyrimidin-7-one.

16. The crystal of PDE5 according to any of the preceding aspects, wherein PDE5 has an active site within the third sub-domain of the protein and comprises Leu 765, Ala 767 and Ile 768 and one or more of Phe 820, Val 782, Phe 786, Tyr 612, Leu 804, Ala 779, Ala 783, Ile 813, Met 816 and Gln 817.

17. The crystal of PDE5 according to aspect 16, wherein the active site is capable of accommodating a pyrazolo-pyrimidinone. An example of a preferred pyrazolo-pyrimidinone is UK-088,800. Another example of a pyrazolo-pyrimidinone is Sildenafil.

18. The crystal of PDE5 according to any of the preceding aspects, wherein said PDE5 has a three-dimensional structure characterised by the atomic co-ordinates set out in Table 4 (PDE5* apo C2 form) or a derivative set as expressed in any reference frame.

19. A heavy atom derivative of the crystal of PDE5 according to any of the preceding aspects.

20. The crystal of PDE5 according to any of the preceding aspects, wherein a PDE5 ligand has been soaked in. The process of soaking may involve transferring the crystal to be soaked from the solution in which it is grown into a stabilising solution containing the ligand or to which the ligand is to be added once the crystal is present in it. It is desirable that the stabilising solution has the physical properties such that the crystal, when transferred, retains its structural integrity and does not crack or dissolve or alter significantly in its crystal parameters, symmetry or cell dimensions.. Preferably, the stabilising solution comprises some or all of the chemical constituents from which the crystal was grown. More preferably, the stabilising solution is of similar or identical composition to the solutions used for crystal growth as set out in aspects 9 to 12 above, although the concentration of precipitant used may be slightly increased. Most preferably the stabilising solution has the same pH as the crystal growth solution. The ligand may be added to the solution containing, or to contain, the crystal for soaking as a solid or in a liquid form (i.e. the ligand already being in solution). Preferably the ligand is in solution and the solvent maybe aqueous, organic or non-organic. Most preferably the ligand is in DMSO. The ligand is commonly added to the soaking solution to yield a final ligand concentration above the expected binding constant for the ligand by PDE5; preferably this is greater than or equal to 10 times the binding constant. The final concentration of ligand present in the crystal soaking solution is preferably between 0.1 and 20 mg/ml, more preferably between 0.5 and 10 mg/ml, and most preferably between 1 and 5 mg/ml.

21. The crystal of PDE5 according to aspect 20, wherein said PDE5 ligand is a PDE5 inhibitor.

22. The crystal of PDE5 according to aspect 21, wherein said PDE5 inhibitor is a pyrazolo-pyrimidinone, preferably UK-088,800. Alternatively the inhibitor is Sildenafil.

23. The crystal of PDE5 according to aspect 22, wherein said PDE5 has a three-dimensional structure characterised by the atomic co-ordinates set out in Table 5 (PDE5*-UK-088,800 complex) or a derivative set as expressed in any reference frame.

24. Use of the atomic co-ordinates determined from the crystal of PDE5 according to any one of the preceding aspects for deriving a three-dimensional structure of a PDE5 or a mutant, derivative, variant, analogue, homologue, sub-domain or fragment thereof.

25. Use according to aspect 24, wherein the PDE5 sub-domain is the catalytic domain.

26. Use of the three-dimensional structure of PDE5 as derivable according to aspect 24 or aspect 25 to computationally or otherwise evaluate the binding interactions of a chemical compound with an active site on PDE5.

27. Use of the three-dimensional structure of PDE5 as derivable according to aspect 26 to design a compound capable of associating with PDE5. Preferably said compound is a pyrazolo-pyrimidinone, more preferably UK-088,800. Alternatively the compound is Sildenafil.

28. Use according to aspect 26 or aspect 27, wherein the compound is a PDE5 ligand.

29. Use according to aspect 28, wherein said compound is a PDE5 inhibitor.

30. Use according to aspect 29, wherein said PDE5 inhibitor is a pyrazolo-pyrimidinone, preferably UK-088,800. Alternatively the inhibitor is Sildenafil.

31. A method of selecting a compound capable of associating with PDE5 from a group of potential PDE5 ligand compounds comprising the following steps:

i. soaking the crystal of PDE5 according to any one of aspects 1 to 19 in a solution containing a potential PDE5 ligand compound;

ii. determining the three-dimensional structure from the soaked crystal; and

iii. assessing whether the compound is bound to PDE5.

32. A compound selected by the use according to aspect 31.

33. A compound designed by the use according to any one of aspects 27 to 30 or identified by the method of aspect 31.

34. The compound according to aspect 33, which is a PDE5 inhibitor.

35. A method of selecting a PDE5 ligand from a group of potential PDE5 ligands, comprising the following steps:

(a) computationally creating a three-dimensional representation of the structure of PDE5 as derivable according to aspect 24 or aspect 25, and a three-dimensional representation of the structure of the potential PDE5 ligand;

(b) co-displaying the three-dimensional representation of the potential PDE5 ligand with the three-dimensional representation of the PDE5 structure; and

(c) assessing whether the three-dimensional representation of the potential PDE5 ligand fits the three-dimensional representation of an active site of the PDE5 structure.

Preferably, ligands are built using molecular graphics tools, more preferably the designed ligand is energy minimised prior to co-display and analysis.

36. The method according to aspect 35, further comprising the following steps:

(d) incorporating the potential PDE5 ligand in a biological PDE5 activity assay; and

(e) determining whether the potential PDE5 ligand modulates PDE5 activity in said assay.

37. The method according to aspect 35 or aspect 36 wherein said potential PDE5 ligand is a potential PDE5 inhibitor compound and said potential PDE5 inhibitor compound inhibits PDE5 activity.

38. A PDE5 ligand selected by the method of any one of aspects 35 to 37. Preferably the ligand is a PDE5 inhibitor.

39. Use of a PDE5 ligand according to aspect 38 as a pharmaceutical. Preferably the ligand is a PDE5 inhibitor.

40. A pharmaceutical composition comprising one or more PDE5 ligands according to aspect 38 and one or more pharmaceutically acceptable excipients.

41. Use of a PDE5 ligand according to aspect 38 in the manufacture of a medicament for the prophylaxis or treatment of a condition, disease, disorder or dysfunction where the inhibition of PDE5 is prophylactically or therapeutically beneficial.

42. Use according to aspect 41, wherein said disorder is a mammalian sexual disorder.

The curative, palliative or prophylactic treatments contemplated by the present invention include the curative, palliative or prophylactic treatment of mammalian sexual disorders, in particular the treatment of mammalian sexual dysfunctions such as male erectile dysfunction (MED), impotence, female sexual dysfunction (FSD), clitoral dysfunction, female hypoactive sexual desire disorder, female sexual arousal disorder (FSAD), female sexual pain disorder or female sexual orgasmic dysfunction (FSOD) as well as sexual dysfunction due to spinal cord injury or selective serotonin re-uptake inhibitor (SSRI) induced sexual dysfunction but, clearly, will also be useful for treating other medical conditions for which PDE5 inhibitor is indicated. Such conditions include premature labour, dysmenorrhoea, benign prostatic hyperplasia (BPH), bladder outlet obstruction, incontinence, stable, unstable and variant (Prinzmetal) angina, hypertension, pulmonary hypertension, chronic obstructive pulmonary disease, coronary artery disease, congestive heart failure, atherosclerosis, conditions of reduced blood vessel patency, e.g. post-percutaneous transluminal coronary angioplasty (post-PTCA), peripheral vascular disease, stroke, nitrate induced tolerance, bronchitis, allergic asthma, chronic asthma, allergic rhinitis, diseases and conditions of the eye such as glaucoma, optic neuropathy, macular degeneration, elevated intra-occular pressure, retinal or arterial occulsion and diseases characterised by disorders of gut motility, e.g. irritable bowel syndrome (IBS).

Further medical conditions for which a PDE5 inhibitor is indicated, and for which treatment with compounds of the present invention may be useful include pre-eclampsia, Kawasaki's syndrome, nitrate tolerance, multiple sclerosis, diabetic nephropathy, neuropathy including autonomic and peripheral neuropathy and in particular diabetic neuropathy and symptoms thereof e.g. gastroparesis, peripheral diabetic neuropathy, Alzheimer's disease, acute respiratory failure, psoriasis, skin necrosis, cancer, metastasis, baldness, nutcracker oesophagus, anal fissure, haemorrhoids, the insulin resistance syndrome, diabetes, hypoxic vasoconstriction as well as the stabilisation of blood pressure during haemodialysis.

Particularly preferred conditions include MED and FSD (preferably FSAD).

Further (numbered) aspects of the present invention include:

43. Use of the atomic co-ordinates determined from the crystal of PDE5 according to any one of aspects 1 to 23, to solve the crystal structure of a mutant, derivative, fragment, variant, analogue, homologue or complex of PDE5.

44. Use of the atomic co-ordinates determined from the crystal of PDE5 according to any one of aspects 1 to 23, to produce a model of the three-dimensional structure of PDE5-related proteins.

45. Use of the three-dimensional structure of PDE5 as derivable according to aspect 24 or aspect 25 to design site-directed mutants that mimic other PDE5 isoforms or variants thereof.

46. A method of soaking a chemical compound into a crystal according to any one of aspects 1 to 19 comprising the following steps:

a) incubating the crystal in an aqueous stabilising solution comprising buffer and polyethylene glycol;

b) combining the chemical compound with the stabilising solution preferably wherein the chemical compound is in solid or liquid form, more preferably in liquid form, most preferably solublised in DMSO; and

c) optionally adding a cryo-protectant to the stabilising solution.

47. A method according to aspect 46, wherein the stabilising solution comprises polyethylene glycol which is of the molecular weight 4000. The polyethylene glycol may be at a concentration of between 10% and 30%, but is most preferably 20%. Additionally the pH is preferably in the range of 7.0 to 7.8; most preferably the pH is 7.4. The buffer should be one capable of providing buffer capacity over the required pH range; preferably HEPES. Preferably the stabilising solution contains an alcohol; most preferably this is isopropanol. Preferably the stabilising solution contains 0.1 M sodium HEPES pH 7.4, 20% polyethylene glycol 4000 and 10% iso-propanol. Examples of suitable cryoprotectants include 2-methyl-2,4-pentanediol (MPD) and organic polymers e.g. lower molecular weight PEG. Carbohydrates such as sorbitol or xylitol, and alcohols may also be used. Preferably the cryoprotectant is glycerol.

A PDE5 ligand (also known as a PDE5 inhibitor compound) according to aspect 38 (hereinafter referred to as “the compound”) can be administered alone but, in human therapy, will generally be administered in admixture with a suitable pharmaceutical excipient diluent or carrier selected with regard to the intended route of administration and standard pharmaceutical practice. Thus, the pharmaceutical compositions, pharmaceuticals and medicaments contemplated by the present invention may be formulated in various ways well-known to one of skill in the art and administered by similarly well-known methods.

For example, the compound of the invention can be administered orally, buccally or sublingually in the form of tablets, capsules (including soft gel capsules), ovules, elixirs, solutions or suspensions, which may contain flavouring or colouring agents, for immediate-, delayed-, modified-, or controlled-release such as sustained-, dual-, or pulsatile delivery applications. The compound may also be administered via intracavemosal injection. The compound may also be administered via fast dispersing or fast dissolving dosages forms or in the form of a high-energy dispersion or as coated particles. Suitable pharmaceutical formulations of the compound may be in coated or un-coated form as desired.

Such tablets may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compound may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate-modifying excipients maybe present both within the dosage form i.e. within the matrix, and/or on the dosage form i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compound can also be administered parenterally, for example, intracavernosally, intravenously, intra-arterially, intraperitoneally, intrathecally, intraventricularly, intraurethrally intrasternally, intracranially, intramuscularly or subcutaneously, or they may be administered by infusion or needleless injection techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

For oral and parenteral administration to human patients, the daily dosage level of the compound will usually be from 10 to 500 mg (in single or divided doses).

Thus, for example, tablets or capsules of the compound may contain from 5 mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, in the treatment of certain conditions (including MED and FSD), the compound may be taken as a single dose on an “as required” basis (i.e. as needed or desired).

The compound can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™ or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

The compound may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the compound can be administered in the form of a suppository or pessary, or they may be applied topically in the form of a gel, hydrogel, lotion, solution, cream, ointment or dusting powder. The compound may also be dermally administered. The compound may also be transdermally administered, for example, by the use of a skin patch. The compound may also be administered by the ocular, pulmonary or rectal routes.

For ophthalmic use, the compound can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, the compound may be formulated in an ointment such as petrolatum.

For application topically to the skin, the compound of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compound may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

Generally, in humans, oral administration the compound is the preferred route, being the most convenient and, for example in MED, avoiding the well-known disadvantages associated with intracavernosal (i.c.) administration. A preferred oral dosing regimen in MED for a typical man is from 25 to 250 mg of compound when required. In circumstances where the recipient suffers from a swallowing disorder or from impairment of drug absorption after oral administration, the drug may be administered parenterally, sublingually or buccally.

For veterinary use, the compound, or a veterinarily acceptable salt thereof, or a veterinarily acceptable solvate or pro-drug thereof, is administered as a suitably acceptable formulation in accordance with normal veterinary practice and the veterinary surgeon will determine the dosing regimen and route of administration which will be most appropriate for a particular animal.

### DETAILED DESCRIPTION OF THE INVENTION

The term “apo” as used herein is taken to mean macromolecule and inparticular any protein (or named protein) that is detached from a/its ligand(s) and/or prosthetic group(s).

The term “buffer” as used herein is taken to include any solution containing a weak acid and a conjugate base of this acid (or, less commonly, a weak base and its conjugate acid). Thus, a “buffer” as used herein resists change in its pH level when an acid or a base is added to it, because the acid neutralises an added base (or, less commonly, the base neutralises an added acid).

An “activity assay” as referred to herein with reference to PDE5 is taken to mean an in vitro assay of PDE inhibitory activities against cyclic guanosine 3′,5′-monophosphate (cGMP) and cyclic adenosine 3′,5′-monophosphate (cAMP) phosphodiesterases which can be determined by measurement of their IC₅₀ values (the concentration of compound required for 50% inhibition of enzyme activity). Preferably the required PDE enzymes can be isolated from a variety of sources, including human corpus cavemosum, human and rabbit platelets, human cardiac ventricle, human skeletal muscle and bovine retina, essentially by a modification of the method of Thompson W J and Appleman M M; Biochemistry 10(2),311-316, 1971, as described by Ballard S A et al.; J. Urology 159(6), 2164-2171, 1998. In particular, cGMP-specific PDE5 and cGMP-inhibited cAMP PDE3 can be obtained from human corpus cavemosum tissue, human platelets or rabbit platelets.

Assays can be performed either using a modification of the “batch” method of Thompson, W J et al.; Biochemistry 18(23), 5228-5237, 1979, essentially as described by Ballard S A et al.; J. Urology 159(6), 2164-2171, 1998, or using a scintillation proximity assay for the direct detection of [³H]-labelled AMP/GMP using a modification of the protocol described by Amersham plc under product code TRKQ7090/7100. In summary, for the scintillation proximity assay, the effect of PDE inhibitors are investigated by assaying a fixed amount of enzyme in the presence of varying inhibitor concentrations and low substrate (cGMP or cAMP in a 3:1 ratio unlabelled to [³H]-labeled at a concentration of ˜⅓ Kₘ or less), such that IC₅≅Kᵢ. The final assay volume is made up to 100 μl with assay buffer [20 mM Tris-HCl pH 7.4, 5mM MgCl₂, 1 mg/ml bovine serum albumin]. Reactions are initiated with enzyme, incubated for 30-60min at 30° C. to give <30% substrate turnover and terminated with 50 μl yttrium silicate SPA beads (containing 3 mM of the respective unlabelled cyclic nucleotide for PDE5). Plates are re-sealed and shaken for 20 min, after which the beads are allowed to settle for 30 min in the dark and then counted on a TopCount plate reader (Packard, Meriden, Conn.). Radioactivity units are converted to % activity of an uninhibited control (100%), plotted against inhibitor concentration, and inhibitor IC₅₀ values obtained using the ° Fit Curve Microsoft Excel extension.

The term “precipitant” as used herein is taken to include any substance that, when added to solution comprising a biological molecule, causes the biological molecule to precipitate or crystallise from the solution.

The term “complex” as used herein is taken to mean a biological macromolecule, preferably a protein, with ligand(s) bound and may be formed before, during or after protein crystallisation.

The term “soaking” as used herein is taken to mean a process of placing a crystal in an aqueous solution containing a chemical compound, (usually) small molecule (e.g. inhibitor), or adding a chemical compound to an aqueous solution containing a crystal such that the compound may be able to enter the crystal lattice by diffusion and may interact (e.g. contact and bond with) with the molecules comprising the lattice preferably to form a protein-ligand complex. The compound may be added to the aqueous solution in solid form, or it may be dissolved in a suitable solvent, preferably di-methyl-sulphoxide (DMSO).

The term “soakable crystal” as used herein is taken to mean a crystal into which a small molecule or ligand may be soaked without significant disruption of the lattice such that the ligand can enter and pass through the crystal lattice and have access to, and may associate with, the molecules comprising the lattice. Preferably a soakable crystal is a crystal which is ameanable to the soaking process in which a small molecule or ligand either in solution or in solid form is added to the solution containing the crystal. The small molecule may then enter the crystal lattice by diffusion and associate with the molecules comprising the crystal lattice. It is preferable that the soakable crystal binds the ligand without disruption of the lattice or cracking of the crystal and that the lattice symmetry and crystal parameters are not significantly altered by the soaking and ligand binding process. It is preferable that the soakable crystal diffracts X-rays to atomic resolution, preferably to beyond 3.5 Å, more preferably beyond 2.5 Å, most preferably 1.5 Å, after undergoing the soaking process.

The term “stabilising solution” as used herein is taken to mean a solution into which a crystal may be transferred for the purposes of further manipulation, for example for soaking, and in which the crystal retains its structural integrity and does not crack or dissolve or alter significantly in its crystal parameters, symmetry or cell dimensions. Preferably the stabilising solution comprises some or all of the chemical constituents from which the crystal was grown and is of the approximately the same, and more preferably identical, pH.

The term “cryoprotectant” as used herein is taken to mean a chemical compound which, when added to a solution, allows the solution to be rapidly frozen without the formation of ice crystals. Such a cryoprotectant is preferably an alcohol such as ethanol or a carbohydrate such as xylitol or sorbitol, but may also be 2-methyl-2,4-pentanediol (MPD) or other organic polymers e.g. lower molecular weight PEG. Most preferably the cryoprotectant is glycerol.

The term “co-crystallisation” as used herein is taken to mean crystallisation of a pre-formed comple of a macromolecule with it's ligand i.e. a protein/small molecule complex.

The terms “mutant”, “variant”, “homologue”, “analogue”, “derivative” or “fragment”, are in relation to the amino acid sequence of the PDE5 protein or polypeptide sequence which is used to produce the crystal of the present invention. The terms include any substitution of, variation of, modification of, replacement of, deletion of, or addition of one (or more) amino acids from (or to) the sequence providing the resultant PDE5 is capable of being crystallised.

Typically, for the “mutant”, “variant”, “homologue”, “analogue”, “derivative” or “fragment” in relation to the amino acid sequence of the protein or polypeptide of the PDE5 of the crystal of the present invention, the types of amino acid substitutions that could be made should maintain the hydrophobicity/hydrophilicity of the amino acid sequence. Amino acid substitutions may be made, for example from 1, 2 or 3 to 10, 20 or 30 substitutions, provided that the modified PDE5 retains the ability to be crystallised in accordance with present invention. Amino acid substitutions may include the use of non-naturally occurring analogues.

In relation to amino acid sequences, the term “variant” as used herein refers to additions, deletions or substitutions of amino acid residues comprised within the wild-type amino acid sequence or fragment thereof. Preferably, a variant in relation to the amino acid sequence of the crystal of the PDE5 of the present invention could include the deletion or substitution of the histidine (His/H) residue as shown emboldened and underlined in SEQ ID NO: 1 (HRGVNNSYIQRSEHPLAQLYCHSIME), which sequence is comprised in the protein or polypeptide of PDE5 of the crystal of the present invention. Replacement of said histidine (H) residue is preferably by way of incorporating one or more amino acid residues (other than histidine), preferably wherein said amino acid residues are neutral or non-polar.

The terms “mutant”, “variant”, “homologue”, “analogue”, “derivative” or “fragment” in relation to the nucleotide sequence coding for the PDE5 of the crystal of the present invention include any substitution of, variation of, modification of, replacement of, deletion of, or addition of, one (or more) nucleotide from (or to) the sequence providing the resultant nucleotide sequence codes for, or is capable of coding for, a PDE5 which is capable of being crystallised.

In relation to nucleotide sequences, the term “variant” as used herein refers to additions, deletions or substitutions of nucleotides of the wild-type nucleotide sequence or fragment thereof.

The term “fragment” as used herein refers to any portion of the PDE5 amino acid sequence as defined in the present invention provided the resultant PDE5 comprising said PDE5 portion is capable of being crystallised. Thus, the term “fragment” also includes PDE5, which comprises any portion of SEQ ID NOS: 1, 2, 3, 4, 5, or 6.

An example of a specific fragment of SEQ ID NO: 6 (full-length “loop-swapped” PDE5 sequence) according to the present invention could be SEQ ID NO: 5 (“loop-swapped” PDE5 catalytic domain). Moreover, an example of a specific fragment of SEQ ID NO: 5 (“loop-swapped” PDE5 catalytic domain) according to the present invention could be SEQ ID NO: 4 (PDE4 “loop region”; HPGVSNQFLINTNSELALMYNDESVLE).

The term “analogue” as used herein means a sequence similar to the amino acid sequence of the PDE5 polypeptide of the crystal of the present invention or of any one of SEQ ID NOS: 1, 2, 3, 4, 5 or 6, but wherein non-detrimental (i.e. not detrimental to the PDE5's capability of being crystallised) amino acid substitutions or deletions have been made.

The term “derivative” as used herein in relation to the amino acid sequence of the PDE5 of the crystal of the present invention, or of any one of SEQ ID NOS: 1, 2, 3, 4, 5 or 6, includes chemical modification of PDE5. Illustrative of such modifications would be replacement of hydrogen by an alkyl, acyl, or amino group.

The term “heavy atom derivative” as used herein in relation to crystals refers to a crystal comprising macromolecules in the crystal lattice which are modified by the inclusion of a heavy atoms (i.e. of significantly greater atomic mass than the atoms common to organic macromolecules, e.g. carbon, nitrogen, oxygen, phosphorous, etc.) into their structure. This is commonly achieved using ionic compounds such as salts of mercury, platinum, gold or silver which may covalently bond to certain groups on the macromolecule either during co-crystalisation with the ionic compound or by soaking the compound into the crystal. Other processes may also be used such as derivitisation with seleno-methionine or with noble gases such as Xenon. The purpose of such heavy atom derivatives of crystals is to provide phasing information for structure solution.

As used herein a “deletion” is defined as a change in either nucleotide or amino acid sequence in which one or more nucleotides or amino acid residues, respectively, are absent.

As used herein an “insertion” or “addition” is a change in a nucleotide or amino acid sequence, which has resulted in the addition of one or more nucleotides or amino acid residues, respectively, as compared to the sequences of the naturally occurring PDE5.

As used herein “substitution” results from the replacement of one or more nucleotides or amino acids by different nucleotides or amino acids, respectively.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The term “homologue” covers homology specifically with respect to protein structure, primary secondary, tertiary and quartemary, and covers any structural PDE5 homologue that is capable of being crystallised.

With respect to homology of the amino acid sequences detailed herein, preferably there is at least 70%, more preferably at least 75%, more preferably at least 80%, yet more preferably at least 85%, even more preferably at least 90% homology to SEQ ID NOS: 1, 2, 3, 4, 5 or 6. More preferably there is at least 95%, and most preferably at least 98%, homology to SEQ ID NOS: 1, 2, 3, 4, 5 or 6.

With respect to homology of the nucleotide sequences coding for the amino acid sequences detailed herein, preferably there is at least 70%, more preferably at least 75%, more preferably at least 80%, yet more preferably at least 85%, even more preferably at least 90% homology to the nucleotide sequences which code for SEQ ID NOS: 1, 2, 3, 4, 5 or 6. More preferably there is at least 95%, and most preferably at least 98%, homology to the nucleotide sequences which code for SEQ ID NOS: 1, 2, 3, 4, 5 or 6.

The term “homologue” with respect to the nucleotide sequence of the PDE5 as defined in the present invention and the amino acid sequence of the PDE5 as defined in the present invention may be synonymous with allelic variations of the sequences.

In particular, the term “homology” as used herein may be equated with the term “identity”. Here, sequence homology with respect to, for example, the amino acid sequence of PDE5 of the crystal of the present invention can be determined by a strict comparison of any one or more of the sequences with another sequence to see if that other sequence has at least 70% identity to the sequence(s). Relative sequence homology (i.e. sequence identity) can also be determined by commercially available computer programs that can calculate percentage (%) homology between two or more sequences. A typical example of such a computer program is CLUSTAL.

Percentage homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for off-line and on-line searching (see Ausubel et al., 1999 ibid, pages 7-58 to 7-60). However, for some applications it is preferred to use the GCG Bestfit program.

Although the final percentage homology can be measured in terms of identity, in some cases, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate percentage homology, preferably percentage sequence identity. The software typically does this as part of the sequence comparison and generates a numerical result.

As indicated, for some applications, sequence homology (or identity) may be determined using any suitable homology algorithm, using for example default parameters. For a discussion of basic issues in similarity searching of sequence databases, see Altschul et al. (1994) Nature Genetics 6:119-129. For some applications, the BLAST algorithm is employed, with parameters set to default values. The BLAST algorithm is described in detail at http://www.ncbi.nih.gov/BLAST/blast_help.html. Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

Other computer program methods to determine identify and similarity between the two sequences include but are not limited to the GCG program package (Devereux et al. 1984 Nucleic Acids Research 12: 387) and FASTA (Atschul et al. 1990 J Molec Biol 403-410).

The amino acid sequence of the PDE5 of the present invention present invention may be produced by expression of a nucleotide sequence coding for the same in a suitable expression system.

In addition, or in the alternative, the protein itself could be produced using chemical methods to synthesize a PDE5 amino acid sequence, in whole or in part. For example, peptides can be synthesized by solid phase techniques, cleaved from the resin, and purified by preparative high performance liquid chromatography (e.g. Creighton (1983) Proteins Structures and Molecular Principles, W H Freeman and Co., New York, N.Y., USA). The composition of the synthetic peptides may be confirmed by amino acid analysis or sequencing (e.g. the Edman degradation procedure).

Direct peptide synthesis can be performed using various solid-phase techniques (Roberge J Y et al, Science, Vol 269, 1995, pp. 202-204) and automated synthesis may be achieved, for example, using the ABI 431 A Peptide Synthesizer (Perkin Elmer, Boston, Mass., USA) in accordance with the instructions provided by the manufacturer. Additionally, the amino acid sequence of PDE5, or any part thereof, may be altered during direct synthesis and/or combined using chemical methods with a sequence from other subunits, or any part thereof, to produce a variant polypeptide.

### Protein Engineering of PDE5*′

Analysis of the catalytic domain of wild-type PDE5 protein by mass spectrometry and SDS-PAGE (data not shown) shows that the protein is cleaved within the region of residues 664-682. High concentrations of protease inhibitors provide some stabilisation of the protein, but cleavage still prevents reproducible crystallisation.

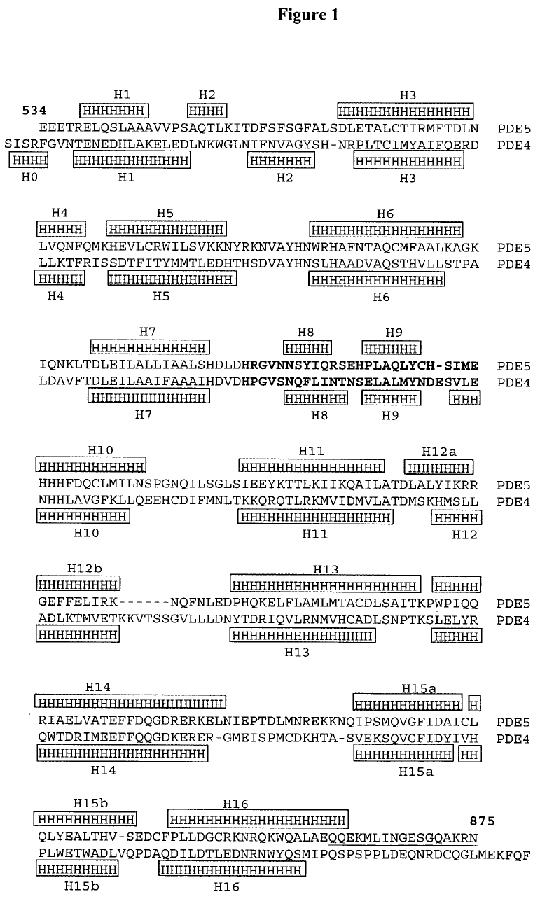

An engineered form of the catalytic domain of PDE5, called PDE5*′, has been produced where the 657-682 region of PDE5 has been replaced by the same region in PDE4 producing a chimeric construct, (see FIG. 1 for sequence alignment of this region). The C-terminus of this construct is also truncated (C-term 858) compared to the wild-type structure (C-term 875). Hereafter this engineered construct will be referred to as PDE5*′.

This engineered protein has been shown to be stable to degradation by mass spectrometry and SDS-PAGE (data not shown). The protein shows improved biophysical properties allowing an alternative purification protocol to be developed. The new protocol utilises binding to a Blue sepharose column and specific elution with cGMP. The wild-type protein had been shown not to bind to this column probably due to the disorder of the structure around the protease cleavage site. This PDE5* protein was used to produce crystals with inhibitors which diffract to higher resolution and have no disordered regions. The protein has also been used reproducibly to produce crystals with further inhibitors which routinely diffract to 1.8 Å resolution or higher, making it an improved protein for use in structure based drug design.

### The Structure of PDE4 and Wild Type PDE5 Catalytic Domains

(This is also detailed in Application Number PCT/IB02/04426, page 28, line 19 to page 29, line 17 and is incorporated herein by reference.)

The structure of the catalytic domain of PDE4b was published (Xu et al. 2000). A topological comparison of the PDE5 catalytic domain with the structures in the Protein Data Bank (PDB) does not reveal significant additional homology with other known protein structures except for the PDE4 structure. Comparisons between the two structures have been made (FIG. 1 of the present application shows a sequence and secondary structural alignment of the two proteins). The structure and domain arrangement is virtually identical to that of PDE4, save that the second sub-domain highlighted in PDE4 is only partially present in the PDE5 structure, as detailed below.

The structure is composed of a single domain of 15 α helices arranged in a compact fold (FIG. 2 of the present application). Within the overall domain, three sub-domains can also be defined. Helices 1 (H1 539-545) and 2 (H2 551-554) lie on the exterior of the protein and comprise the N-terminal region of the construct. These two helices do not overlay with the equivalent ones (H0, H1 and H2) in the PDE4 structure. This region is not well conserved across the PDE protein family. Helices 3 (H3 568-582), 4 (H4 584-588), 5 (H5 592-604), 6 (H6 615-631) and 7 (H7 640-652) form the first sub-domain of the protein and are contained within the core of the protein. There is no observable electron density for helices 8 and 9 based on the PDE4 nomenclature. Helix 10 (H10 684-694) is again on the exterior and forms the dimer interface within the structure. Helices 10 and 11 (H11 706-721) are the visible portion of the second sub-domain. Helices 12 (H12a 725-731, H12b 733-741), 13 (H13 749-765), 14 (H14 772-797), 15 (H15 813-824), 16 (H16 826-836) and 17 (H17 841-861) form the third sub-domain of the protein. It should be noted that in PDE5 helix H12 is not a contiguous helix as in PDE4 but is composed of two short helices with a kink in the middle and helix H15 is a contiguous helix in PDE5 but not in PDE4.

### Structure of PDE5* Catalytic Domain Complexed with UK-088,800

The structure of the catalytic domain of PDE5* protein complexed with UK-088,800 was determined by molecular replacement using one molecule of the protein structure from the PDE5* complex with Sildenafil (as described in Application Number PCT/IB02/04426: Example 11, “crystallisation of PDE5* with Sildenafil”, page 46, lines 8 to 25; Example 15, “data collection, structure determination and refinement PDE5* with Sildenafil”, page 50, line 9 to page 51, line 6 and Table 6, “atomic co-ordinates for baculovirus-expressed PDE5* complex with Sildenafil”, pages 328 to 408; incorporated herein by reference) as a basis for the search model, the co-ordinates for which are included in Table 3 of the present application. The PDE5* catalytic domain crystallises in the space group C2 as a monomer with one molecule present in the asymmetric unit. The C2 form of the PDE5* catalytic domain crystallises in the space group C2 with approximate cell dimensions a=55.97 Å, b=76.55 Å, c=80.50 Å and β=103.09. They contain one molecule per asymmetric unit and have a solvent content of 44% (Matthews, 1969). The structure of the PDE5* comprises 17 α helices and the overall fold is very similar to the wild-type structure with a number of important differences. The major difference in the structure is the presence of helices H8 and H9 composed of the swapped portion from PDE4, residues 657-682. These fold in an identical way to that observed in the PDE4 structure and complete the second sub-domain of the protein. The entire C-terminal region of this construct can also be built into the electron density leaving just three disordered residues at the N-terminus of this structure. This is likely to contribute to its enhanced properties for crystallisation.

### PDE5*: Active Site and Protein-Inhibitor Interactions

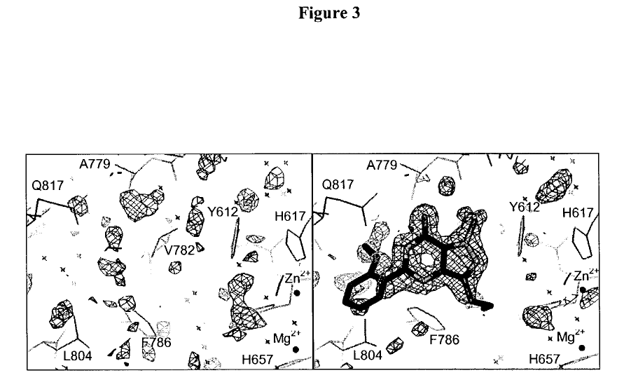

UK-088,800 occupies the same region of the active site as observed for the previously described UK-092,480, Sildenafil, as detailed below (Application Number PCT/IB02/04426: FIG. 4, “A view of compound Sildenafil bound to loop swapped PDE5 (PDE5*), page 425; incorporated herein by reference) and demonstrates the same mainly hydrophobic interactions with the protein (FIG. 3 “Comparison of the fo-fc electron density map contoured at 2.5σ of apo PDE5* (left) and PDE5* soaked with UK-088,800 (right), at the active site region” of the present application). The same two direct hydrogen bonds observed in the complex of PDE5* with Sildenafil are also observed between Gln 817 of the protein and inhibitor (Oε1-N4 2.9 Å and Nε2-O1 3.1 Å).

Carbon atom C7 of the inhibitor points into a small hydrophobic pocket formed by Leu 765, Ala 767 and Ile 768. These residues, together with Phe 820, form a planar face to the binding site against which the purine ring of the inhibitor stacks. The opposite side of the purine packs against Val 782. The C10 propyl substituent form good van der Waals contacts with Phe 786. Phe 786 packs against Leu 804 which in turn forms additional hydrophobic interactions with the phenyl moiety of the inhibitor. The O-alkyl moiety occupies a small pocket bounded by Ala 779, Phe 786, Ala 783, Val 782, Leu 804, Ile 813, Met 816 and Gln 817. There is no direct interaction between the inhibitor and the zinc ion found in the active site. The structure confirms the competitive nature of the mode of inhibition of UK-088,800 by binding in the active site, therefore blocking access for the cGMP substrate (which has also been modelled—data not included).

The main difference comparing Sildenafil and UK-088,800 is that the latter inhibitor lacks the sulphonylpiperazine present in Sildenafil which points out of the active site.

### Wild-Type PDE5-Sildenafil Complex: Active Site and Protein-Inhibitor Interactions

(This is detailed in Application Number PCT/EB02/04426, page 30, line 8 to page 31, line 1, and is incorporated herein by reference.)

The active site lies mainly within the third sub-domain of the protein and is bounded by helices H15, H14, the C-terminus of H13, and the C-terminus of H11, along with the loop region between H11and H12a. The majority of the interactions between the inhibitor and the protein are hydrophobic in nature, with only two direct hydrogen bonds observed (Application Number PCT/IB02/04426: FIG. 3, page 424, incorporated herein by reference). The first is between N17 of the purine ring of the inhibitor and Oε1 of Gln 817 (2.8 Å) and the second is from the adjacent oxygen atom O16 of the inhibitor to Nε2 of the same residue Gln 817 (3.1 Å).

Carbon atom C12 of the inhibitor points into a small hydrophobic pocket formed by Leu 765, Ala 767 and Ile 768. These residues, together with Phe 820, form a planar face to the binding site against which the purine ring of the inhibitor stacks. The opposite side of the purine packs against Val 782. The C5 propyl substituent forms good van der Waals contacts with Val 782 and Phe 786 and Tyr 612. Phe 786 and Leu 804 form additional hydrophobic interactions with the phenyl moiety of the inhibitor. The O-alkyl moiety occupies a small pocket bounded by Ala 779, Phe 786, Ala 783, Val 782, Leu 804, Ile 813, Met 816 and Gln 817. The sulphonamide group points out towards the solvent whilst the piperazine ring is bounded by the extended residues 662-665, although whether the conformation of this part of the structure is unaffected by the chain break is questionable. There is no direct interaction between the inhibitor and the zinc ion found in the active site. The structure confirms the competitive nature of the mode of inhibition of Sildenafil by binding in the active site therefore blocking access for the cGMP substrate (which has also been modelled - data not included).

### Wild Type PDE5-Sildenafil Complex: Metal Ions in the Active Site

Only one zinc atom is present in the active site of this structure. The co-ordination of the ion within the active site is also consistent with that expected for zinc. The metal is co-ordinated by His 653 (Nε2-Zn 2.0 Å), His 617 (Nε2-Zn 2.1 Å), Asp 764 (OD2-Zn 2.2 Å) and also Asp 654 (OD2-Zn 2.2 Å). These residues are completely conserved across the PDE gene family. There is no evidence of a second metal ion in the active site.

The present invention will now be described, by way of example only, with reference to the accompanying Sequence Listing and Figures, in which:

SEQ ID NO: 1 shows the amino acid sequence of the loop region from PDE5.

SEQ ID NO: 2 shows the amino acid sequence of the wild-type PDE5 catalytic domain.

SEQ ID NO: 3 shows the amino acid sequence of the full-length wild-type PDE5 sequence.

SEQ ID NO: 4 shows the amino acid sequence of the loop region of PDE4.

SEQ ID NO: 5 shows the amino acid sequence of the loop-swapped PDE5 catalytic domain=PDE5*.

SEQ ID NO: 6 shows the amino acid sequence of full-length PDE5 sequence comprising PDE5*.

SEQ ID NOS: 7-14 are oligonucleotide primers.

FIG. 1 shows an alignment of PDE5 (upper sequence) and PDE4b (lower sequence) catalytic domains. Positions and numbering of helices from the structures are marked for each. Residues in bold show a sequence alignment for the engineered region. The sequence from PDE4 has been used to replace the corresponding region in PDE5. This results in a residue insertion in this region. Underlining highlight C-terminal region absent in PDE5*.

FIG. 2 shows a ribbon representation of the overall fold of proteins showing secondary structure elements. The inhibitor is shown in an all atom stick representation and the metal ions as spheres. (A)=PDE4b, (B)=wild-type PDE5+Sildenafil, (C)=“loop-swapped” PDE5 (PDE5*)+Sildenafil. Helices are numbered using PDE4 structure as reference. Helices H0-H7 form sub-domain 1, helices H8-H11 form sub-domain 2, and helices H12-H16 form sub-domain 3.

FIG. 3: Comparison of the fo-fc electron density map contoured at 2.5 σ of apo PDE5* (“loop-swapped” PDE5) (left) and PDE5* soaked with UK-088,800 (right), at the active site region.

FIG. 4: A two dimensional representation of the structure of UK-088,800, 5-(2-ethoxyphenyl)-1 -methyl-3-propyl- 1,6-dihydro-7H-pyrazolo[4,3-d]pyrimidin-7-one (only the Hydrogen on N4 is illustrated).

### EXPERIMENTAL SECTION

### EXAMPLE 1

### Construction and Expression of PDE5 Wild-Type Catalytic Domain (E534-N875)

Oligonucleotide primers were designed from the sequence of human PDE5 (Accession number=AB001635). DNA fragments were generated by PCR amplification from a full-length PDE5 clone. The following oligonucleotides were used:

PDE5 5′ Untagged Oligo:

(SEQ ID NO:7)

CGTGAATTCATGGAGGAAGAAACAAGAGAGCTAC
PDE5 3′ Long Oligo:

(SEQ ID NO:8)

CGTTCTAGACTATCAGTTCCGCTTGGCCTGGCCGCTTTCCCC

The PCR reaction was carried out for 30 cycles in a total volume of 50 μl in a solution containing 1.5 mM MgCl₂, 200 μM dNTPs, 50 pmol of each primer and 2.5 units of Expand DNA polymerase (Roche, East Sussex, UK). Each cycle was 94° C., 1 min, 50° C., 1 min and 72° C., 2 mins.

The final amplified DNA fragments for both constructs were separated on a 1% agarose gel and purified using a QIAquick gel extraction kit (Qiagen, West Sussex, UK). The fragment was then digested using EcoRI and XbaI, and ligated into pFastbac1 EcoRI/XbaI-digested vector (Life Technologies, Paisley, UK). The ligation was carried out at 12° C. for 16 hours. The ligation mix was then electroporated into E. coli (TOP 10) (Invitrogen, Gronigen, The Netherlands).

Clones containing the desired insert were selected by using 2YT plates containing 100 μg/ml ampicillin and checked using endonuclease digestion for presence of correct size insert. DNA sequence analysis was carried out by Lark (Saffron Waldon, UK).

Recombinant bacmid DNA was produced by transforming E.coli DH10BAC™ with pFastbac1::PDE5 catalytic domain (534-875) plasmid DNA. This was carried out according to the method shown in the Bac to Bac™ baculovirus expression manual (Life Technologies, Paisley, UK). PCR analysis was used to verify successful transposition to the bacmid using pUC/M13 amplification primers (Invitrogen, Gronigen, The Netherlands).

Generation of primary baculovirus stocks was carried out by transfection using Sf-9 insect cells. Bacmid DNA containing the correct insert was mixed with CELLFECTIN™ transfection reagent (Life Technologies, Paisley, UK) and added to a monolayer of Sf-9 insect cells using SF-900II serum free medium (Invitrogen, Gronigen, The Netherlands). Following 72 hours incubation at 27° C. the supernatant was harvested as the initial baculovirus stock. This stock was amplified by adding the initial virus stock into a suspension of Sf-9 insect cells at 1×10⁶ cells/ml in 1 litre Erlenmeyer flasks (Coming Life Sciences, New York, USA), at an agitation of 125 rpm at 27° C. After 6 days post infection the supernatant was harvested by centrifugation and stored at 4° C. as the working virus stock. The titre of this working stock was determined by conventional plaque assay analysis as in the Bac to Bac baculovirus expression manual (Invitrogen, Gronigen, The Netherlands).

Protein expression was optimised in Erlenmeyer flask cultures using Sf-9 and T.ni High5 insect cell cultures looking at different multiplicity's of infection (MOI) and harvest times, the optimal conditions found were then scaled up into fermenters.

The fermenters used were autoclavable Applikon 3 litre stirred vessels controlled using Applikon 1030 biocontrollers. Inoculum of T.ni High5 cells was initially prepared from shake flask cultures. The fermenter was inoculated with 5×10⁵ cells/ml, with an initial working volume of 1.8 litres made up in Excel 405 serum free medium (JRH Biosciences, Kansas, USA). Temperature was controlled at 27° C., dissolved oxygen concentration controlled at 60% and pH was measured but not controlled. Oxygen concentration was controlled throughout. Agitation was set at 150 rpm with a double impeller system of marine impeller within the culture and Rushton impeller at the liquid/headspace interface. Aeration was continuous to the headspace at 0.5 l/min.

When the viable cell density reached 2×10⁶ cells/ml the culture was infected using an MOI of 1 from the titred baculovirus working stock. Harvest time for the culture was 48 hours post infection. This was achieved by centrifugation at 2000 g for 15 mins; the insect cell pellet was then stored at −80° C. prior to purification.

### EXAMPLE 2

### Construction and Expression of His6-Tagged PDE5 Wild-Type Catalytic Domain (E534-N875)

Oligonucleotide primers were designed from the sequence of human PDE5 (=PDE5A1 isoform; Accession number=AB001635). DNA fragments were generated by PCR amplification from a full-length PDE5 clone. The following oligonucleotides were used:

| | |
|---|---|
| PDE5 5′ His6 Oligo | |
| CGTGAATTCATGCATCATCATCATCATCATCTTCTGGTTCCGCGTGGATCTG | (SEQ ID NO:9) |
| CGCCCGAGGAAGAAACAAGAGAGCTAC | |
| PDE5 3′ Long Oligo | |
| CGTTCTAGACTATCAGTTCCGCTTGGCCTGGCCGCTTTCCCC | (SEQ ID NO:8) |

The PCR reaction was carried out for 30 cycles in a total volume of 50 μl in a solution containing 1.5 nM MgCl₂, 200 μM dNTPs, 50 pmol of each primer and 2.5 units of Expand DNA polymerase (Roche, East Sussex, UK). Each cycle was 94° C., 1 min, 50° C., 1 min and 72° C., 2 mins.

The final amplified DNA fragments for both constructs were separated on a 1% agarose gel and purified using a QIAquick gel extraction kit (Qiagen, West Sussex, UK). The fragment was then digested using EcoRI and XbaI, and ligated into pFastbac1 EcoRI/XbaI-digested vector (Life Technologies, Paisley, UK). The ligation was carried out at 12° C. for 16 hours. The ligation mix was then electroporated into E. Coli (TOP 10) (Invitrogen, Gronigen, The Netherlands).

Clones containing the desired insert were selected by using 2YT plates containing 100 μg/ml ampicillin and checked using endonuclease digestion for presence of correct size insert. DNA sequence analysis was carried out by Lark (Saffron Waldon, UK).

Methods to generate the recombinant baculovirus were as those for wild-type PDE5 catalytic domain (see EXAMPLE 1).

Expression optimisation again showed T.ni High5 insect cells to give the best expression. Therefore baculovirus expression in fermenters was carried out using the same procedures as for the previous construct.

### EXAMPLE 3

### Construction and Expression of PDE5* (E534-E858) in Baculovirus

The PDE5* construct was produced by using overlap extension PCR where the following oligonucleotides were used:

| | |
|---|---|
| A: CGTGAATTCATGGAGGAAGAAACAAGAGAGCTAC | (SEQ ID NO:7) |
| B: CAAAGAAAGTTCTGAATTTGTGTTGATGAGAAACTGATTGGAGACTCCA | (SEQ ID NO:10) |
| GGATGATCCAAATCGTGGCTTAG | |
| C: ATCAACACAAATTCAGAACTTGCTTTGATGTATAATGATGAATCTGTGTT | (SEQ ID NO:11) |
| GGAACACCATCATTTTGACCAG | |
| D: CGTTCTAGACTATCATTCTGCAAGGGCCTGCCATTTCTG | (SEQ ID NO:12) |

Initial DNA fragments were generated using oligonucleotides A+B and C+D with the same template DNA as for the wild-type PDE5 catalytic domain construct. The PCR reaction was carried out for 30 cycles in a total volume of 50 μl in a solution containing 1.5 mM MgCl₂, 200 μM dNTPs, 50 pmol of each primer and 2 units of Expand DNA polymerase (Roche, East Sussex, UK). Each cycle was 94° C., 1 min, 50° C., 2 min, and 72° C., 3 min. In the second round of PCR, DNA products from PCR A+B and C+D were used as template DNA with the oligonucleotides A+D used to amplify the full-length construct. The PCR conditions were the same as the initial PCR reaction. This generates a construct with the PDE4 swapped region and a C-terminal truncation (C-term 858) as compared to PDE5 catalytic domain (C-term 875).

Methods to generate the recombinant baculovirus were as those for wild-type PDE5 catalytic domain (see EXAMPLE 1).

Expression optimisation again showed T.ni High5 insect cells to give the best expression. Therefore baculovirus expression in fermenters was carried out using the same procedures as for the previous construct.

### EXAMPLE 4

### Construction and Expression of PDE5* (E534-E858) in E. coli

The PDE5* construct in E. coli was produced by using PCR where the following oligonucleotides were used and the template DNA being pFastbac1::PDE5* plasmid DNA (sequence verified), produced in EXAMPLE 3.

(SEQ ID NO:13)

E: CGTCATATGGAGGAAGAAACAAGAGAGCTAC

(SEQ ID NO:14)

F: CGTCTCGAGCTATCATTCTGCAAGGGCCTGCCATTTCTG

The PCR reaction was carried out for 30 cycles in a total volume of 50 μl in a solution containing 1.5 mM MgCl₂, 200 μM dNTPs, 50 pmol of each primer and 2.5 units of Expand DNA polymerase (Roche, East Sussex, UK). Each cycle was 94° C., 1 min, 50° C., 1 min and 72° C., 2 mins.

The final amplified DNA fragment was separated on a 1% agarose gel and purified using a QIAquick gel extraction kit (Qiagen, West Sussex, UK). The fragments were then digested using Nde1 and Xho1, and ligated into pET21C (Novagen, Nottingham, UK) Nde1/Xho1-digested vector. The ligation was carried out at 12° C. for 16 hours. The ligation mix was then electroporated into E. coli (TOP 10) (Invitrogen, Gronigen, The Netherlands).

Clones containing the desired insert were selected by using 2YT plates containing 100 μg/ml ampicillin. Plasmid DNA was also checked using endonuclease digestion for presence of correct size insert. DNA sequence analysis was carried out by Lark (Saffron Waldon, UK).

The correctly sequenced plasmid DNA was then electroporated into E. coli BL21 (DE3) (Novagen, Nottingham, UK) for expression. Expression was carried out in 7 litre Applikon fermenters using 5 litre 2YT broth containing 100μg/ml carbenicillin as the medium. Agitation was set at 1000 rpm using a double rushton impeller assembly and aeration to the sparger at 2 litres/min. The fermenter was inoculated with an overnight shake flask culture grown at 37° C. and 200 rpm, the inoculation density was 1 % vol/vol. The fermentation was pH controlled at 7.2 using 20% vol/vol NH₄OH solution and temperature initially set to 37° C. When the OD_{600 nm} reached 1.5 the temperature set-point was reduced to 25° C. and then the culture was induced with IPTG at a final concentration of 1 mM. The fermentation was then harvested 4 hours post-induction by batch centrifugation (8,000 rpm for 10 minutes). The final pellet was then frozen (−80° C.) to await subsequent purification.

### EXAMPLE 5

### Purification of PDE5* Catalytic Domain

Pellet from the E. coli fermentation was resuspended into 10 mls lysis buffer per gram wet cell weight and mechanically broken using a continuous cell disrupter (Constant Systems, Warwickshire, UK) at a pressure of 20 kpsi. The lysis buffer consisted of 50 mM Tris HCl (pH 7.5), 100 mM NaCl, 1 mM DTT containing EDTA-free protease inhibitor cocktail tablets (Roche, East Sussex, UK) and 10 μM E-64. The lysate was chilled and centrifuged at 14000 g for 45 min to remove cell debris. All purifications were subsequently carried out using an Äkta Explorer purification system (Amersham Pharmacia, Buckinghamshire, UK). The supernatant was applied to a 50 ml Q—sepharose fast-flow column (Amersham Pharmacia, Buckinghamshire, UK) at 5 ml/min with the flow-through collected. The flow-through sample was then applied at 50 ml/min to a 2 litre G-25 superfine desalting column (Amersham Pharmacia, Buckinghamshire, UK) pre-equilibrated in Blue sepharose buffer A (50 mM Bis-Tris (pH 6.4), 50 mM NaCl, 2 mM EDTA, 2 mM EGTA and 1 mM DTT). The protein fraction was eluted in Blue sepharose buffer A.

The next column step was carried out in series, loading the sample initially onto a 20 ml SP-sepharose high performance column (Amersham Pharmacia, Buckinghamshire, UK) then flow-through from this directly onto a 10 ml Blue sepharose fast-flow column (Amersham Pharmacia, Buckinghamshire, UK) at a flow-rate of 2 ml/min. Once loading was complete, the SP-sepharose column was taken out of line and the Blue sepharose column washed with 5 column volumes of Blue sepharose buffer A. The column was washed with Blue sepharose buffer A containing 1 M NaCl until the absorbance 280 nm reached baseline and then washed with 5 column volumes of Blue sepharose buffer A. PDE5* protein was step-eluted using Blue sepharose buffer containing 20 mM cGMP (Na-salt) (Sigma, Dorset, UK). Fractions were assayed on Tris-glycine SDS gels (Invitrogen, Gronigen, The Netherlands) and pooled accordingly. These fractions were concentrated to 2.5 mg/ml using centrifugal concentrators (Vivascience, Gloucestershire, UK) and loaded at 1.5 ml/min onto a Superdex-200 prep grade 26/60 column pre-equilibrated with 50 mM Bis-Tris (pH 6.8), 500 mM NaCl, 1 mM DTT and 2 μM E-64. The eluted fractions were analysed on Tris-glycine SDS PAGE gels.

### EXAMPLE 6

### Crystallisation of PDE5*

The PDE5* fractions from the final gel filtration column were pooled (total volume of 25 mls) and the protein concentration was assayed (0.2 mg/ml). The protein solution was supplemented with 10 μM E-64 and 1 mg/ml leupeptin (Sigma, Dorset, UK). The solution was concentrated to 10 mg/ml using a Centriprep 10 kDa Molecular weight cut-off centrifugal concentrator (Amicon Bioseparations, Maine, USA) at 3,000rpm, 4° C. Prior to crystallisation, the protein solution was centrifuged for 5 min at 14,000 rpm in an Eppendorf centrifuge.

Hanging drop vapour diffusion crystallisation trials were set up at 4° C. Drops comprised of 2 μl reservoir buffer mixed with 2 μl protein solution were suspended on cover slips over 500 μl reservoir solutions containing 0.1M sodium HEPES pH 7.4, 20% Polyethylene glycol 4000 and 10% iso-Propanol. Plate-shaped crystals, up to 700 μm in largest dimension, grew after 1-3 days. Crystals were transferred to a 20 μl drop of 0.1 M sodium HEPES pH 7.4 and 20% Polyethylene glycol 4000 in square microbridges. Crystals were then transferred to a solution of 0.1 M sodium HEPES pH 7.4, 20% Polyethylene glycol 4000 with 15% glycerol and then frozen during X-ray data collection.

The crystals belong to space group C2 with cell dimensions a=55.97 Å, b=76.55 Å, c=80.50 Å and β=103.09. They contain one molecule per asymmetric unit and have a solvent content of 44% (Matthews, 1969).

### EXAMPLE 7

### Soaking Inhibitor into PDE5*

Plate-shaped crystals of apo PDE5* obtained according to methods detailed in EXAMPLE 6 were transferred to a 20 μ;l drop of 0.1M sodium HEPES and 20% Polyethylene glycol 4000 in square microbridges. To this, 1.0 μl of a 20 mg/ml concentration of inhibitor dissolved in DMSO was added. Crystals were then transferred to a solution of 0.1M sodium HEPES pH 7.4, 20% Polyethylene glycol 4000 with 15% glycerol and then frozen during X-ray data collection.

### EXAMPLE 8

### Data Collection, Structure Determination and Refinement of the Soakable C2 Crystal form of PDE5*

The structure of the E. coli-engineered PDE5* was solved by molecular replacement (MR) using the coordinates of one subunit of PDE5* in the P2₁ crystal form (the crystals belong to the monoclinic space group P2₁ with unit cell dimensions a=54.93 Å, b=77.77 Å, c=82.05 Å, α=γ90° β100.9550°) the coordinate set is detailed in Table 3. Molecular replacement was performed using AMORE (CCP4, J.Navaza 1994). The resulting map was of good quality and the structure was refitted using QUANTA® (Accelrys products).

A representative X-ray diffraction data set was collected with a Rigaku Satum92 CCD detector on an in-house FR-D rotating anode (Rigaku), with Osmic mirrors (MSC). Data were processed using the CrystalClear/D*Trek processing package (Rigaku-MSC). Data collection statistics are summarised in Table 1 (Apo).

The crystals belong to space group C2 with cell dimensions a=55.97 Å, b=76.55 Å, c=80.50 Å and β=103.09. They contain one molecule per asymmetric unit and have a solvent content of 44% (Matthews, 1968).

Refinement was carried out in the resolution range 30-1.6 Å using CNX (Brüinger et al., 1998) with the “mlf” maximum likelihood target function. Partial structure factors from a flat bulk-solvent model and anisotropic B-factor correction were supplied throughout the refinement. The R-factor for the refined model is 0.231 (free R-factor, 5% of data, 0.267) for all data in the resolution range 30-1.6 Å. The refinement statistics are summarised in Table 2 (Apo).

The co-ordinate set for the refined model of apo PDE5* is recorded in Table 4. The model contains 323 amino acid residues, 537-858 (residue Glu 681A has been numbered to maintain PDE5 numbering scheme), a zinc ion and a magnesium ion, as well as 222 water molecules.

### EXAMPLE 9

### Data Collection, Structure Determination and Refinement of the Soakable C2 Crystal form of PDE5* Soaked with UK-088,800

The structure of the UK-088,800 soaked into the C2 crystal form of PDE5* was solved by difference Fourier methods.

X-ray diffraction data were collected with an RaxisIV image plate detector on an in-house FR-D rotating anode (Rigaku), with Osmic mirrors (MSC). All data were processed using the HKL package (Otwinowski & Minor, 1997). Data collection statistics are summarised in Table 1 (UK-088,800).

Refinement was carried out in the resolution range 30-1.5 Å using CNX (Brüinger et al., 1998) with the “mlf” maximum likelihood target function. Partial structure factors from a flat bulk-solvent model and anisotropic B-factor correction were supplied throughout the refinement. The R-factor for the refined model is 0.219 (free R-factor, 5% of data, 0.228) for all data in the resolution range 30-1.5 Å. The refinement statistics are summarised in Table 2 (UK-088,800).

The co-ordinate set for the refined model of PDE5*-UK-088,800 is recorded in Table 5. The model contains 323 amino acid residues, 537-858 (residue Glu 681A has been numbered to maintain PDE5 numbering scheme), the ligand UK-088,800, a zinc ion and a magnesium ion, as well as 183 water molecules.

### EXAMPLE 10

### Comparison of the apo PDE5* and PDE5* Soaked with UK-088,800

The (fo-fc)α_{calc} electron density map of PDE5* soaked with UK-088,800 calculated with experimental structure factor amplitudes and model phases derived prior to incorporation of the ligand into the model, shows a clearly featured peak that can unequivocally be interpreted as UK-088,880 (FIG. 3). In contrast, the equivalent (fo-FC)α_{calc} electron density map of apo PDE5* shows no significant residual features that could be interpreted as a small molecule ligand. The experiment allowed the experimental determination of the binding mode of UK-088,800 to PDE5* and demonstrates that the C2 crystal form of PDE5* as described herein is amenable to ligand soaking. The structure of PDE5* in the C2 crystal form does not show significant differences to that observed with the P2₁ crystal form.

TABLE 1
Data collection statistics for the soakable C2
crystal form of PDE5*

| | Apo | UK-088,800 |
|---|---|---|
| Resolution [Å] | 1.6 | 1.5 |
| Unique reflections | 41189 | 52038 |
| Completeness [%] | 94.5 | 99.5 |
| Redundancy | 2.9 | 3.5 |
| R_{sym}^{a} | 0.074 | 0.049 |

${}^{a} {R}_{sym} = {\sum }_{hkl} {\sum }_{i} \vert {I}_{i} ( hkl ) - \overline{I ( hkl )} \vert / {\sum }_{hkl} {\sum }_{i} \vert {I}_{i} ( hkl ) \vert$

TABLE 2
Refinement statistics of soakable C2 crystal form of PDE5*

| | Apo | UK-088,800 |
|---|---|---|
| Resolution range, Å | 30.0-1.6 | 30.0-1.5 |
| Amino acid residues, no. | 323 | 323 |
| Water molecules, no. | 222 | 183 |
| rmsd bond length, Å | 0.005 | 0.005 |
| rmsd bond angles, ° | 1.130 | 1.158 |
| ^{a}R-factor | 0.231 | 0.219 |
| Free R-factor (5 % of data) | 0.267 | 0.228 |

${}^{a} R = {\sum }_{hkl} ‖ {F}_{obs} \vert - k \vert {F}_{calc} ‖ / {\sum }_{hkl} \vert {F}_{obs} \vert$

TABLE 3
Atomic coordinates for baculovirus-expressed PDE5*
complex with Sildenafil Atom Number, Atom Type,
Residue Type, Residue Number Cartesian Coordinates
X, Y, Z, Atom Occupancy (O) and Temperature Factor (B)

| | | | | | | X | Y | Z | O | B |
|---|---|---|---|---|---|---|---|---|---|---|
| ATOM | 1 | CB | THR | A | 537 | −3.008 | 28.295 | 13.745 | 1.00 | 40.04 |
| ATOM | 2 | OG1 | THR | A | 537 | −4.423 | 28.364 | 13.527 | 1.00 | 40.20 |
| ATOM | 3 | CG2 | THR | A | 537 | −2.273 | 28.516 | 12.435 | 1.00 | 40.01 |
| ATOM | 4 | C | THR | A | 537 | −2.764 | 26.998 | 15.853 | 1.00 | 42.52 |
| ATOM | 5 | O | THR | A | 537 | −3.657 | 26.395 | 16.451 | 1.00 | 44.33 |
| ATOM | 6 | N | THR | A | 537 | −3.513 | 25.856 | 13.760 | 1.00 | 44.29 |
| ATOM | 7 | CA | THR | A | 537 | −2.652 | 26.924 | 14.338 | 1.00 | 43.47 |
| ATOM | 8 | N | ARG | A | 538 | −1.857 | 27.751 | 16.466 | 1.00 | 33.65 |
| ATOM | 9 | CA | ARG | A | 538 | −1.818 | 27.897 | 17.916 | 1.00 | 31.51 |
| ATOM | 10 | CB | ARG | A | 538 | −3.201 | 28.267 | 18.463 | 1.00 | 51.67 |
| ATOM | 11 | CG | ARG | A | 538 | −3.420 | 27.913 | 19.928 | 1.00 | 52.25 |
| ATOM | 12 | CD | ARG | A | 538 | −2.377 | 28.532 | 20.845 | 1.00 | 52.37 |
| ATOM | 13 | NE | ARG | A | 538 | −1.851 | 27.547 | 21.788 | 1.00 | 52.59 |
| ATOM | 14 | CZ | ARG | A | 538 | −1.048 | 27.833 | 22.809 | 1.00 | 52.58 |
| ATOM | 15 | NH1 | ARG | A | 538 | −0.676 | 29.087 | 23.032 | 1.00 | 52.27 |
| ATOM | 16 | NH2 | ARG | A | 538 | −0.608 | 26.864 | 23.600 | 1.00 | 52.33 |
| ATOM | 17 | C | ARG | A | 538 | −1.336 | 26.584 | 18.521 | 1.00 | 30.67 |
| ATOM | 18 | O | ARG | A | 538 | −0.175 | 26.465 | 18.906 | 1.00 | 30.03 |
| ATOM | 19 | N | GLU | A | 539 | −2.221 | 25.594 | 18.593 | 1.00 | 27.90 |
| ATOM | 20 | CA | GLU | A | 539 | −1.827 | 24.310 | 19.151 | 1.00 | 27.13 |
| ATOM | 21 | CB | GLU | A | 539 | −3.000 | 23.322 | 19.146 | 1.00 | 33.40 |
| ATOM | 22 | CG | GLU | A | 539 | −2.627 | 21.946 | 19.679 | 1.00 | 34.46 |
| ATOM | 23 | CD | GLU | A | 539 | −3.814 | 21.007 | 19.761 | 1.00 | 34.35 |
| ATOM | 24 | OE1 | GLU | A | 539 | −4.656 | 21.037 | 18.843 | 1.00 | 34.50 |
| ATOM | 25 | OE2 | GLU | A | 539 | −3.899 | 20.233 | 20.738 | 1.00 | 34.57 |
| ATOM | 26 | C | GLU | A | 539 | −0.666 | 23.734 | 18.346 | 1.00 | 27.02 |
| ATOM | 27 | O | GLU | A | 539 | 0.280 | 23.189 | 18.909 | 1.00 | 27.32 |
| ATOM | 28 | N | LEU | A | 540 | −0.739 | 23.866 | 17.027 | 1.00 | 26.77 |
| ATOM | 29 | CA | LEU | A | 540 | 0.320 | 23.354 | 16.166 | 1.00 | 26.36 |
| ATOM | 30 | CB | LEU | A | 540 | −0.070 | 23.496 | 14.695 | 1.00 | 24.87 |
| ATOM | 31 | CG | LEU | A | 540 | 1.027 | 23.155 | 13.682 | 1.00 | 24.86 |
| ATOM | 32 | CD1 | LEU | A | 540 | 1.426 | 21.689 | 13.833 | 1.00 | 25.12 |
| ATOM | 33 | CD2 | LEU | A | 540 | 0.531 | 23.447 | 12.264 | 1.00 | 25.63 |
| ATOM | 34 | C | LEU | A | 540 | 1.631 | 24.091 | 16.415 | 1.00 | 26.72 |
| ATOM | 35 | O | LEU | A | 540 | 2.681 | 23.479 | 16.603 | 1.00 | 26.47 |
| ATOM | 36 | N | GLN | A | 541 | 1.573 | 25.416 | 16.407 | 1.00 | 25.80 |
| ATOM | 37 | CA | GLN | A | 541 | 2.777 | 26.196 | 16.626 | 1.00 | 26.31 |
| ATOM | 38 | CB | GLN | A | 541 | 2.455 | 27.682 | 16.484 | 1.00 | 28.46 |
| ATOM | 39 | CG | GLN | A | 541 | 2.334 | 28.108 | 15.029 | 1.00 | 28.55 |
| ATOM | 40 | CD | GLN | A | 541 | 1.709 | 29.475 | 14.868 | 1.00 | 28.37 |
| ATOM | 41 | OE1 | GLN | A | 541 | 1.843 | 30.334 | 15.736 | 1.00 | 28.46 |
| ATOM | 42 | NE2 | GLN | A | 541 | 1.033 | 29.687 | 13.747 | 1.00 | 29.26 |
| ATOM | 43 | C | GLN | A | 541 | 3.415 | 25.894 | 17.978 | 1.00 | 25.96 |
| ATOM | 44 | O | GLN | A | 541 | 4.636 | 25.785 | 18.082 | 1.00 | 26.05 |
| ATOM | 45 | N | SER | A | 542 | 2.589 | 25.733 | 19.008 | 1.00 | 27.15 |
| ATOM | 46 | CA | SER | A | 542 | 3.103 | 25.440 | 20.341 | 1.00 | 27.52 |
| ATOM | 47 | CB | SER | A | 542 | 1.980 | 25.510 | 21.379 | 1.00 | 38.20 |
| ATOM | 48 | OG | SER | A | 542 | 1.535 | 26.841 | 21.558 | 1.00 | 38.87 |
| ATOM | 49 | C | SER | A | 542 | 3.752 | 24.067 | 20.390 | 1.00 | 27.50 |
| ATOM | 50 | O | SER | A | 542 | 4.795 | 23.888 | 21.015 | 1.00 | 27.75 |
| ATOM | 51 | N | LEU | A | 543 | 3.131 | 23.095 | 19.732 | 1.00 | 23.90 |
| ATOM | 52 | CA | LEU | A | 543 | 3.676 | 21.743 | 19.723 | 1.00 | 23.62 |
| ATOM | 53 | CB | LEU | A | 543 | 2.702 | 20.785 | 19.022 | 1.00 | 31.43 |
| ATOM | 54 | CG | LEU | A | 543 | 2.929 | 19.274 | 19.167 | 1.00 | 32.27 |
| ATOM | 55 | CD1 | LEU | A | 543 | 1.959 | 18.529 | 18.263 | 1.00 | 31.54 |
| ATOM | 56 | CD2 | LEU | A | 543 | 4.340 | 18.904 | 18.800 | 1.00 | 32.06 |
| ATOM | 57 | C | LEU | A | 543 | 5.025 | 21.725 | 19.009 | 1.00 | 23.65 |
| ATOM | 58 | O | LEU | A | 543 | 5.998 | 21.144 | 19.498 | 1.00 | 23.61 |
| ATOM | 59 | N | ALA | A | 544 | 5.079 | 22.365 | 17.848 | 1.00 | 24.41 |
| ATOM | 60 | CA | ALA | A | 544 | 6.295 | 22.402 | 17.052 | 1.00 | 24.73 |
| ATOM | 61 | CB | ALA | A | 544 | 6.014 | 23.090 | 15.728 | 1.00 | 23.31 |
| ATOM | 62 | C | ALA | A | 544 | 7.477 | 23.076 | 17.748 | 1.00 | 25.97 |
| ATOM | 63 | O | ALA | A | 544 | 8.619 | 22.642 | 17.600 | 1.00 | 25.19 |
| ATOM | 64 | N | ALA | A | 545 | 7.202 | 24.125 | 18.512 | 1.00 | 29.21 |
| ATOM | 65 | CA | ALA | A | 545 | 8.259 | 24.861 | 19.196 | 1.00 | 30.03 |
| ATOM | 66 | CB | ALA | A | 545 | 7.837 | 26.315 | 19.351 | 1.00 | 32.13 |
| ATOM | 67 | C | ALA | A | 545 | 8.660 | 24.288 | 20.553 | 1.00 | 30.21 |
| ATOM | 68 | O | ALA | A | 545 | 9.698 | 24.657 | 21.105 | 1.00 | 30.14 |
| ATOM | 69 | N | ALA | A | 546 | 7.849 | 23.382 | 21.088 | 1.00 | 28.91 |
| ATOM | 70 | CA | ALA | A | 546 | 8.126 | 22.790 | 22.391 | 1.00 | 28.92 |
| ATOM | 71 | CB | ALA | A | 546 | 6.878 | 22.068 | 22.913 | 1.00 | 27.14 |
| ATOM | 72 | C | ALA | A | 546 | 9.308 | 21.831 | 22.400 | 1.00 | 29.12 |
| ATOM | 73 | O | ALA | A | 546 | 9.648 | 21.222 | 21.381 | 1.00 | 29.57 |
| ATOM | 74 | N | VAL | A | 547 | 9.936 | 21.702 | 23.564 | 1.00 | 27.29 |
| ATOM | 75 | CA | VAL | A | 547 | 11.060 | 20.791 | 23.714 | 1.00 | 26.19 |
| ATOM | 76 | CB | VAL | A | 547 | 11.917 | 21.148 | 24.947 | 1.00 | 37.00 |
| ATOM | 77 | CG1 | VAL | A | 547 | 12.925 | 20.042 | 25.223 | 1.00 | 37.18 |
| ATOM | 78 | CG2 | VAL | A | 547 | 12.635 | 22.464 | 24.711 | 1.00 | 37.04 |
| ATOM | 79 | C | VAL | A | 547 | 10.482 | 19.395 | 23.903 | 1.00 | 26.09 |
| ATOM | 80 | O | VAL | A | 547 | 9.473 | 19.226 | 24.588 | 1.00 | 27.35 |
| ATOM | 81 | N | VAL | A | 548 | 11.110 | 18.400 | 23.286 | 1.00 | 28.09 |
| ATOM | 82 | CA | VAL | A | 548 | 10.638 | 17.029 | 23.409 | 1.00 | 28.48 |
| ATOM | 83 | CB | VAL | A | 548 | 10.792 | 16.254 | 22.079 | 1.00 | 23.53 |
| ATOM | 84 | CG1 | VAL | A | 548 | 10.198 | 14.845 | 22.215 | 1.00 | 24.59 |
| ATOM | 85 | CG2 | VAL | A | 548 | 10.123 | 17.016 | 20.953 | 1.00 | 23.63 |
| ATOM | 86 | C | VAL | A | 548 | 11.468 | 16.336 | 24.475 | 1.00 | 28.05 |
| ATOM | 87 | O | VAL | A | 548 | 12.637 | 16.033 | 24.252 | 1.00 | 29.14 |
| ATOM | 88 | N | PRO | A | 549 | 10.879 | 16.080 | 25.655 | 1.00 | 28.24 |
| ATOM | 89 | CD | PRO | A | 549 | 9.550 | 16.472 | 26.151 | 1.00 | 28.37 |
| ATOM | 90 | CA | PRO | A | 549 | 11.647 | 15.414 | 26.709 | 1.00 | 28.05 |
| ATOM | 91 | CB | PRO | A | 549 | 10.648 | 15.321 | 27.858 | 1.00 | 28.48 |
| ATOM | 92 | CG | PRO | A | 549 | 9.781 | 16.524 | 27.640 | 1.00 | 28.59 |
| ATOM | 93 | C | PRO | A | 549 | 12.133 | 14.045 | 26.254 | 1.00 | 28.74 |
| ATOM | 94 | O | PRO | A | 549 | 11.639 | 13.495 | 25.262 | 1.00 | 27.64 |
| ATOM | 95 | N | SER | A | 550 | 13.103 | 13.505 | 26.977 | 1.00 | 30.38 |
| ATOM | 96 | CA | SER | A | 550 | 13.658 | 12.202 | 26.649 | 1.00 | 30.47 |
| ATOM | 97 | CB | SER | A | 550 | 14.923 | 11.963 | 27.464 | 1.00 | 32.70 |
| ATOM | 98 | OG | SER | A | 550 | 14.611 | 11.959 | 28.843 | 1.00 | 32.28 |
| ATOM | 99 | C | SER | A | 550 | 12.654 | 11.101 | 26.955 | 1.00 | 29.91 |
| ATOM | 100 | O | SER | A | 550 | 11.683 | 11.310 | 27.684 | 1.00 | 30.19 |
| ATOM | 101 | N | ALA | A | 551 | 12.898 | 9.922 | 26.396 | 1.00 | 27.46 |
| ATOM | 102 | CA | ALA | A | 551 | 12.022 | 8.784 | 26.626 | 1.00 | 27.31 |
| ATOM | 103 | CB | ALA | A | 551 | 12.451 | 7.610 | 25.756 | 1.00 | 33.59 |
| ATOM | 104 | C | ALA | A | 551 | 12.064 | 8.389 | 28.097 | 1.00 | 27.99 |
| ATOM | 105 | O | ALA | A | 551 | 11.031 | 8.093 | 28.700 | 1.00 | 27.97 |
| ATOM | 106 | N | GLN | A | 552 | 13.264 | 8.382 | 28.670 | 1.00 | 30.15 |
| ATOM | 107 | CA | GLN | A | 552 | 13.433 | 8.019 | 30.074 | 1.00 | 31.28 |
| ATOM | 108 | CB | GLN | A | 552 | 14.917 | 8.099 | 30.457 | 1.00 | 50.35 |
| ATOM | 109 | CG | GLN | A | 552 | 15.547 | 9.470 | 30.254 | 1.00 | 51.01 |
| ATOM | 110 | CD | GLN | A | 552 | 17.068 | 9.439 | 30.310 | 1.00 | 51.35 |
| ATOM | 111 | OE1 | GLN | A | 552 | 17.725 | 8.932 | 29.398 | 1.00 | 51.08 |
| ATOM | 112 | NE2 | GLN | A | 552 | 17.635 | 9.979 | 31.385 | 1.00 | 51.12 |
| ATOM | 113 | C | GLN | A | 552 | 12.595 | 8.934 | 30.969 | 1.00 | 31.11 |
| ATOM | 114 | O | GLN | A | 552 | 11.888 | 8.470 | 31.868 | 1.00 | 30.93 |
| ATOM | 115 | N | THR | A | 553 | 12.660 | 10.232 | 30.704 | 1.00 | 32.81 |
| ATOM | 116 | CA | THR | A | 553 | 11.904 | 11.202 | 31.484 | 1.00 | 32.49 |
| ATOM | 117 | CB | THR | A | 553 | 12.250 | 12.637 | 31.060 | 1.00 | 35.77 |
| ATOM | 118 | OG1 | THR | A | 553 | 13.615 | 12.915 | 31.399 | 1.00 | 35.76 |
| ATOM | 119 | CG2 | THR | A | 553 | 11.339 | 13.639 | 31.761 | 1.00 | 35.80 |
| ATOM | 120 | C | THR | A | 553 | 10.399 | 10.999 | 31.333 | 1.00 | 32.89 |
| ATOM | 121 | O | THR | A | 553 | 9.658 | 11.065 | 32.313 | 1.00 | 33.04 |
| ATOM | 122 | N | LEU | A | 554 | 9.954 | 10.750 | 30.105 | 1.00 | 29.86 |
| ATOM | 123 | CA | LEU | A | 554 | 8.534 | 10.545 | 29.827 | 1.00 | 28.95 |
| ATOM | 124 | CB | LEU | A | 554 | 8.278 | 10.693 | 28.325 | 1.00 | 27.17 |
| ATOM | 125 | CG | LEU | A | 554 | 8.586 | 12.088 | 27.769 | 1.00 | 27.34 |
| ATOM | 126 | CD1 | LEU | A | 554 | 8.439 | 12.101 | 26.255 | 1.00 | 27.36 |
| ATOM | 127 | CD2 | LEU | A | 554 | 7.650 | 13.105 | 28.410 | 1.00 | 27.14 |
| ATOM | 128 | C | LEU | A | 554 | 8.011 | 9.197 | 30.326 | 1.00 | 28.80 |
| ATOM | 129 | O | LEU | A | 554 | 6.803 | 9.014 | 30.486 | 1.00 | 28.57 |
| ATOM | 130 | N | LYS | A | 555 | 8.925 | 8.260 | 30.561 | 1.00 | 24.74 |
| ATOM | 131 | CA | LYS | A | 555 | 8.588 | 6.935 | 31.071 | 1.00 | 25.34 |
| ATOM | 132 | CB | LYS | A | 555 | 7.764 | 7.072 | 32.356 | 1.00 | 44.27 |
| ATOM | 133 | CG | LYS | A | 555 | 8.420 | 7.960 | 33.402 | 1.00 | 45.10 |
| ATOM | 134 | CD | LYS | A | 555 | 7.451 | 8.363 | 34.501 | 1.00 | 45.29 |
| ATOM | 135 | CE | LYS | A | 555 | 8.106 | 9.352 | 35.455 | 1.00 | 45.10 |
| ATOM | 136 | NZ | LYS | A | 555 | 7.173 | 9.818 | 36.518 | 1.00 | 45.31 |
| ATOM | 137 | C | LYS | A | 555 | 7.818 | 6.084 | 30.060 | 1.00 | 24.58 |
| ATOM | 138 | O | LYS | A | 555 | 7.148 | 5.116 | 30.433 | 1.00 | 25.45 |
| ATOM | 139 | N | ILE | A | 556 | 7.932 | 6.430 | 28.783 | 1.00 | 26.12 |
| ATOM | 140 | CA | ILE | A | 556 | 7.222 | 5.699 | 27.740 | 1.00 | 26.45 |
| ATOM | 141 | CB | ILE | A | 556 | 7.160 | 6.515 | 26.432 | 1.00 | 24.50 |
| ATOM | 142 | CG2 | ILE | A | 556 | 6.294 | 7.753 | 26.640 | 1.00 | 24.65 |
| ATOM | 143 | CG1 | ILE | A | 556 | 8.571 | 6.907 | 25.987 | 1.00 | 24.30 |
| ATOM | 144 | CD1 | ILE | A | 556 | 8.631 | 7.477 | 24.582 | 1.00 | 24.18 |
| ATOM | 145 | C | ILE | A | 556 | 7.806 | 4.321 | 27.446 | 1.00 | 26.74 |
| ATOM | 146 | O | ILE | A | 556 | 7.203 | 3.525 | 26.722 | 1.00 | 26.46 |
| ATOM | 147 | N | THR | A | 557 | 8.974 | 4.027 | 28.004 | 1.00 | 26.58 |
| ATOM | 148 | CA | THR | A | 557 | 9.571 | 2.718 | 27.782 | 1.00 | 26.91 |
| ATOM | 149 | CB | THR | A | 557 | 11.098 | 2.750 | 27.964 | 1.00 | 33.52 |
| ATOM | 150 | OG1 | THR | A | 557 | 11.653 | 3.811 | 27.179 | 1.00 | 33.53 |
| ATOM | 151 | CG2 | THR | A | 557 | 11.709 | 1.430 | 27.506 | 1.00 | 33.78 |
| ATOM | 152 | C | THR | A | 557 | 8.983 | 1.711 | 28.766 | 1.00 | 26.91 |
| ATOM | 153 | O | THR | A | 557 | 9.108 | 0.501 | 28.578 | 1.00 | 27.08 |
| ATOM | 154 | N | ASP | A | 558 | 8.327 | 2.213 | 29.810 | 1.00 | 22.97 |
| ATOM | 155 | CA | ASP | A | 558 | 7.739 | 1.347 | 30.824 | 1.00 | 24.55 |
| ATOM | 156 | CB | ASP | A | 558 | 7.564 | 2.101 | 32.145 | 1.00 | 42.16 |
| ATOM | 157 | CG | ASP | A | 558 | 8.816 | 2.834 | 32.573 | 1.00 | 43.08 |
| ATOM | 158 | OD1 | ASP | A | 558 | 9.918 | 2.258 | 32.459 | 1.00 | 43.16 |
| ATOM | 159 | OD2 | ASP | A | 558 | 8.692 | 3.987 | 33.036 | 1.00 | 43.34 |
| ATOM | 160 | C | ASP | A | 558 | 6.386 | 0.797 | 30.408 | 1.00 | 23.87 |
| ATOM | 161 | O | ASP | A | 558 | 5.522 | 1.537 | 29.940 | 1.00 | 24.35 |
| ATOM | 162 | N | PHE | A | 559 | 6.200 | −0.505 | 30.593 | 1.00 | 26.69 |
| ATOM | 163 | CA | PHE | A | 559 | 4.936 | −1.140 | 30.257 | 1.00 | 26.24 |
| ATOM | 164 | CB | PHE | A | 559 | 5.052 | −2.662 | 30.372 | 1.00 | 34.04 |
| ATOM | 165 | CG | PHE | A | 559 | 5.885 | −3.290 | 29.292 | 1.00 | 34.73 |
| ATOM | 166 | CD1 | PHE | A | 559 | 5.537 | −3.135 | 27.952 | 1.00 | 34.65 |
| ATOM | 167 | CD2 | PHE | A | 559 | 7.018 | −4.036 | 29.610 | 1.00 | 34.64 |
| ATOM | 168 | CE1 | PHE | A | 559 | 6.304 | −3.713 | 26.943 | 1.00 | 34.43 |
| ATOM | 169 | CE2 | PHE | A | 559 | 7.793 | −4.618 | 28.610 | 1.00 | 34.92 |
| ATOM | 170 | CZ | PHE | A | 559 | 7.435 | −4.456 | 27.272 | 1.00 | 34.80 |
| ATOM | 171 | C | PHE | A | 559 | 3.841 | −0.639 | 31.196 | 1.00 | 26.88 |
| ATOM | 172 | O | PHE | A | 559 | 2.664 | −0.631 | 30.837 | 1.00 | 25.97 |
| ATOM | 173 | N | SER | A | 560 | 4.245 | −0.213 | 32.389 | 1.00 | 25.49 |
| ATOM | 174 | CA | SER | A | 560 | 3.321 | 0.285 | 33.407 | 1.00 | 26.93 |
| ATOM | 175 | CB | SER | A | 560 | 3.945 | 0.120 | 34.797 | 1.00 | 39.67 |
| ATOM | 176 | OG | SER | A | 560 | 5.170 | 0.830 | 34.899 | 1.00 | 40.02 |
| ATOM | 177 | C | SER | A | 560 | 2.920 | 1.745 | 33.197 | 1.00 | 26.06 |
| ATOM | 178 | O | SER | A | 560 | 2.205 | 2.327 | 34.018 | 1.00 | 26.04 |
| ATOM | 179 | N | PHE | A | 561 | 3.382 | 2.326 | 32.098 | 1.00 | 26.05 |
| ATOM | 180 | CA | PHE | A | 561 | 3.081 | 3.711 | 31.751 | 1.00 | 25.67 |
| ATOM | 181 | CB | PHE | A | 561 | 3.551 | 3.982 | 30.321 | 1.00 | 26.66 |
| ATOM | 182 | CG | PHE | A | 561 | 3.158 | 5.330 | 29.795 | 1.00 | 25.48 |
| ATOM | 183 | CD1 | PHE | A | 561 | 3.990 | 6.430 | 29.972 | 1.00 | 26.67 |
| ATOM | 184 | CD2 | PHE | A | 561 | 1.948 | 5.502 | 29.127 | 1.00 | 26.67 |
| ATOM | 185 | CE1 | PHE | A | 561 | 3.624 | 7.686 | 29.489 | 1.00 | 26.12 |
| ATOM | 186 | CE2 | PHE | A | 561 | 1.571 | 6.748 | 28.642 | 1.00 | 25.76 |
| ATOM | 187 | CZ | PHE | A | 561 | 2.412 | 7.844 | 28.823 | 1.00 | 26.46 |
| ATOM | 188 | C | PHE | A | 561 | 1.597 | 4.082 | 31.865 | 1.00 | 26.23 |
| ATOM | 189 | O | PHE | A | 561 | 0.715 | 3.293 | 31.533 | 1.00 | 25.55 |
| ATOM | 190 | N | SER | A | 562 | 1.335 | 5.304 | 32.320 | 1.00 | 24.91 |
| ATOM | 191 | CA | SER | A | 562 | −0.029 | 5.803 | 32.463 | 1.00 | 26.24 |
| ATOM | 192 | CB | SER | A | 562 | −0.406 | 5.906 | 33.940 | 1.00 | 42.72 |
| ATOM | 193 | OG | SER | A | 562 | −1.720 | 6.414 | 34.084 | 1.00 | 43.29 |
| ATOM | 194 | C | SER | A | 562 | −0.134 | 7.179 | 31.817 | 1.00 | 24.23 |
| ATOM | 195 | O | SER | A | 562 | 0.730 | 8.024 | 32.013 | 1.00 | 25.59 |
| ATOM | 196 | N | ASP | A | 563 | −1.199 | 7.401 | 31.055 | 1.00 | 23.00 |
| ATOM | 197 | CA | ASP | A | 563 | −1.410 | 8.675 | 30.373 | 1.00 | 22.76 |
| ATOM | 198 | CB | ASP | A | 563 | −2.135 | 8.446 | 29.043 | 1.00 | 25.24 |
| ATOM | 199 | CG | ASP | A | 563 | −3.635 | 8.228 | 29.230 | 1.00 | 23.77 |
| ATOM | 200 | OD1 | ASP | A | 563 | −4.037 | 7.150 | 29.718 | 1.00 | 23.35 |
| ATOM | 201 | OD2 | ASP | A | 563 | −4.421 | 9.143 | 28.906 | 1.00 | 24.81 |
| ATOM | 202 | C | ASP | A | 563 | −2.263 | 9.659 | 31.176 | 1.00 | 22.96 |
| ATOM | 203 | O | ASP | A | 563 | −2.297 | 10.848 | 30.865 | 1.00 | 21.79 |
| ATOM | 204 | N | PHE | A | 564 | −2.958 | 9.150 | 32.187 | 1.00 | 24.29 |
| ATOM | 205 | CA | PHE | A | 564 | −3.878 | 9.962 | 32.987 | 1.00 | 25.23 |
| ATOM | 206 | CB | PHE | A | 564 | −4.394 | 9.157 | 34.180 | 1.00 | 41.24 |
| ATOM | 207 | CG | PHE | A | 564 | −5.576 | 9.787 | 34.857 | 1.00 | 41.58 |
| ATOM | 208 | CD1 | PHE | A | 564 | −6.793 | 9.895 | 34.191 | 1.00 | 41.99 |
| ATOM | 209 | CD2 | PHE | A | 564 | −5.469 | 10.300 | 36.146 | 1.00 | 42.28 |
| ATOM | 210 | CE1 | PHE | A | 564 | −7.888 | 10.506 | 34.797 | 1.00 | 41.91 |
| ATOM | 211 | CE2 | PHE | A | 564 | −6.558 | 10.915 | 36.763 | 1.00 | 41.92 |
| ATOM | 212 | CZ | PHE | A | 564 | −7.769 | 11.018 | 36.086 | 1.00 | 41.83 |
| ATOM | 213 | C | PHE | A | 564 | −3.410 | 11.321 | 33.490 | 1.00 | 25.10 |
| ATOM | 214 | O | PHE | A | 564 | −4.185 | 12.278 | 33.488 | 1.00 | 25.27 |
| ATOM | 215 | N | GLU | A | 565 | −2.158 | 11.406 | 33.921 | 1.00 | 29.45 |
| ATOM | 216 | CA | GLU | A | 565 | −1.618 | 12.651 | 34.459 | 1.00 | 30.36 |
| ATOM | 217 | CB | GLU | A | 565 | −0.456 | 12.341 | 35.408 | 1.00 | 40.02 |
| ATOM | 218 | CG | GLU | A | 565 | −0.821 | 11.524 | 36.645 | 1.00 | 40.24 |
| ATOM | 219 | CD | GLU | A | 565 | −1.312 | 10.120 | 36.320 | 1.00 | 40.61 |
| ATOM | 220 | OE1 | GLU | A | 565 | −0.655 | 9.424 | 35.514 | 1.00 | 40.05 |
| ATOM | 221 | OE2 | GLU | A | 565 | −2.352 | 9.711 | 36.883 | 1.00 | 40.18 |
| ATOM | 222 | C | GLU | A | 565 | −1.144 | 13.652 | 33.409 | 1.00 | 30.08 |
| ATOM | 223 | O | GLU | A | 565 | −0.860 | 14.810 | 33.732 | 1.00 | 30.87 |
| ATOM | 224 | N | LEU | A | 566 | −1.070 | 13.217 | 32.155 | 1.00 | 25.91 |
| ATOM | 225 | CA | LEU | A | 566 | −0.587 | 14.073 | 31.078 | 1.00 | 25.51 |
| ATOM | 226 | CB | LEU | A | 566 | 0.157 | 13.219 | 30.051 | 1.00 | 30.68 |
| ATOM | 227 | CG | LEU | A | 566 | 1.256 | 12.294 | 30.578 | 1.00 | 31.45 |
| ATOM | 228 | CD1 | LEU | A | 566 | 1.775 | 11.429 | 29.437 | 1.00 | 30.44 |
| ATOM | 229 | CD2 | LEU | A | 566 | 2.377 | 13.112 | 31.186 | 1.00 | 31.07 |
| ATOM | 230 | C | LEU | A | 566 | −1.642 | 14.889 | 30.349 | 1.00 | 25.31 |
| ATOM | 231 | O | LEU | A | 566 | −2.793 | 14.475 | 30.220 | 1.00 | 24.57 |
| ATOM | 232 | N | SER | A | 567 | −1.228 | 16.054 | 29.859 | 1.00 | 27.06 |
| ATOM | 233 | CA | SER | A | 567 | −2.110 | 16.928 | 29.096 | 1.00 | 26.87 |
| ATOM | 234 | CB | SER | A | 567 | −1.619 | 18.373 | 29.159 | 1.00 | 26.86 |
| ATOM | 235 | OG | SER | A | 567 | −0.406 | 18.519 | 28.439 | 1.00 | 26.48 |
| ATOM | 236 | C | SER | A | 567 | −2.038 | 16.447 | 27.650 | 1.00 | 26.54 |
| ATOM | 237 | O | SER | A | 567 | −1.187 | 15.617 | 27.314 | 1.00 | 25.78 |
| ATOM | 238 | N | ASP | A | 568 | −2.921 | 16.963 | 26.800 | 1.00 | 25.65 |
| ATOM | 239 | CA | ASP | A | 568 | −2.935 | 16.588 | 25.388 | 1.00 | 25.44 |
| ATOM | 240 | CB | ASP | A | 568 | −4.077 | 17.292 | 24.657 | 1.00 | 35.03 |
| ATOM | 241 | CG | ASP | A | 568 | −5.437 | 16.732 | 25.028 | 1.00 | 35.82 |
| ATOM | 242 | OD1 | ASP | A | 568 | −6.450 | 17.284 | 24.555 | 1.00 | 36.02 |
| ATOM | 243 | OD2 | ASP | A | 568 | −5.489 | 15.744 | 25.788 | 1.00 | 35.24 |
| ATOM | 244 | C | ASP | A | 568 | −1.604 | 16.946 | 24.739 | 1.00 | 26.02 |
| ATOM | 245 | O | ASP | A | 568 | −1.040 | 16.154 | 23.981 | 1.00 | 24.45 |
| ATOM | 246 | N | LEU | A | 569 | −1.104 | 18.140 | 25.040 | 1.00 | 26.56 |
| ATOM | 247 | CA | LEU | A | 569 | 0.177 | 18.584 | 24.501 | 1.00 | 27.05 |
| ATOM | 248 | CB | LEU | A | 569 | 0.544 | 19.958 | 25.066 | 1.00 | 35.85 |
| ATOM | 249 | CG | LEU | A | 569 | 1.958 | 20.460 | 24.751 | 1.00 | 36.55 |
| ATOM | 250 | CD1 | LEU | A | 569 | 2.110 | 20.683 | 23.255 | 1.00 | 36.24 |
| ATOM | 251 | CD2 | LEU | A | 569 | 2.211 | 21.755 | 25.509 | 1.00 | 36.44 |
| ATOM | 252 | C | LEU | A | 569 | 1.267 | 17.582 | 24.864 | 1.00 | 25.81 |
| ATOM | 253 | O | LEU | A | 569 | 2.080 | 17.214 | 24.021 | 1.00 | 27.30 |
| ATOM | 254 | N | GLU | A | 570 | 1.283 | 17.151 | 26.126 | 1.00 | 24.45 |
| ATOM | 255 | CA | GLU | A | 570 | 2.266 | 16.183 | 26.596 | 1.00 | 23.61 |
| ATOM | 256 | CB | GLU | A | 570 | 2.143 | 15.984 | 28.104 | 1.00 | 28.33 |
| ATOM | 257 | CG | GLU | A | 570 | 2.600 | 17.182 | 28.912 | 1.00 | 28.49 |
| ATOM | 258 | CD | GLU | A | 570 | 2.473 | 16.953 | 30.402 | 1.00 | 28.93 |
| ATOM | 259 | OE1 | GLU | A | 570 | 1.346 | 16.679 | 30.864 | 1.00 | 27.96 |
| ATOM | 260 | OE2 | GLU | A | 570 | 3.500 | 17.049 | 31.111 | 1.00 | 29.89 |
| ATOM | 261 | C | GLU | A | 570 | 2.149 | 14.832 | 25.892 | 1.00 | 23.15 |
| ATOM | 262 | O | GLU | A | 570 | 3.166 | 14.194 | 25.622 | 1.00 | 23.63 |
| ATOM | 263 | N | THR | A | 571 | 0.931 | 14.382 | 25.592 | 1.00 | 21.78 |
| ATOM | 264 | CA | THR | A | 571 | 0.803 | 13.099 | 24.897 | 1.00 | 21.61 |
| ATOM | 265 | CB | THR | A | 571 | −0.670 | 12.597 | 24.814 | 1.00 | 20.96 |
| ATOM | 266 | OG1 | THR | A | 571 | −1.477 | 13.550 | 24.117 | 1.00 | 22.41 |
| ATOM | 267 | CG2 | THR | A | 571 | −1.242 | 12.365 | 26.212 | 1.00 | 20.91 |
| ATOM | 268 | C | THR | A | 571 | 1.362 | 13.236 | 23.483 | 1.00 | 22.39 |
| ATOM | 269 | O | THR | A | 571 | 1.924 | 12.283 | 22.929 | 1.00 | 21.33 |
| ATOM | 270 | N | ALA | A | 572 | 1.218 | 14.425 | 22.906 | 1.00 | 21.89 |
| ATOM | 271 | CA | ALA | A | 572 | 1.726 | 14.683 | 21.564 | 1.00 | 22.51 |
| ATOM | 272 | CB | ALA | A | 572 | 1.235 | 16.037 | 21.061 | 1.00 | 23.95 |
| ATOM | 273 | C | ALA | A | 572 | 3.247 | 14.647 | 21.603 | 1.00 | 23.15 |
| ATOM | 274 | O | ALA | A | 572 | 3.885 | 14.120 | 20.690 | 1.00 | 23.62 |
| ATOM | 275 | N | LEU | A | 573 | 3.830 | 15.211 | 22.660 | 1.00 | 24.60 |
| ATOM | 276 | CA | LEU | A | 573 | 5.281 | 15.215 | 22.805 | 1.00 | 25.21 |
| ATOM | 277 | CB | LEU | A | 573 | 5.694 | 16.061 | 24.013 | 1.00 | 27.78 |
| ATOM | 278 | CG | LEU | A | 573 | 5.470 | 17.565 | 23.808 | 1.00 | 27.76 |
| ATOM | 279 | CD1 | LEU | A | 573 | 5.732 | 18.313 | 25.101 | 1.00 | 27.67 |
| ATOM | 280 | CD2 | LEU | A | 573 | 6.391 | 18.074 | 22.699 | 1.00 | 27.52 |
| ATOM | 281 | C | LEU | A | 573 | 5.777 | 13.784 | 22.953 | 1.00 | 25.21 |
| ATOM | 282 | O | LEU | A | 573 | 6.797 | 13.413 | 22.374 | 1.00 | 25.36 |
| ATOM | 283 | N | CYS | A | 574 | 5.052 | 12.979 | 23.725 | 1.00 | 21.84 |
| ATOM | 284 | CA | CYS | A | 574 | 5.432 | 11.581 | 23.897 | 1.00 | 21.46 |
| ATOM | 285 | CB | CYS | A | 574 | 4.446 | 10.852 | 24.807 | 1.00 | 23.13 |
| ATOM | 286 | SG | CYS | A | 574 | 4.663 | 11.173 | 26.560 | 1.00 | 22.80 |
| ATOM | 287 | C | CYS | A | 574 | 5.426 | 10.890 | 22.540 | 1.00 | 21.90 |
| ATOM | 288 | O | CYS | A | 574 | 6.277 | 10.047 | 22.260 | 1.00 | 21.49 |
| ATOM | 289 | N | THR | A | 575 | 4.449 | 11.240 | 21.705 | 1.00 | 23.44 |
| ATOM | 290 | CA | THR | A | 575 | 4.334 | 10.631 | 20.384 | 1.00 | 21.88 |
| ATOM | 291 | CB | THR | A | 575 | 3.006 | 11.042 | 19.699 | 1.00 | 21.68 |
| ATOM | 292 | OG1 | THR | A | 575 | 1.916 | 10.641 | 20.536 | 1.00 | 22.63 |
| ATOM | 293 | CG2 | THR | A | 575 | 2.857 | 10.370 | 18.340 | 1.00 | 22.72 |
| ATOM | 294 | C | THR | A | 575 | 5.534 | 11.000 | 19.518 | 1.00 | 22.93 |
| ATOM | 295 | O | THR | A | 575 | 6.111 | 10.140 | 18.865 | 1.00 | 20.91 |
| ATOM | 296 | N | ILE | A | 576 | 5.929 | 12.268 | 19.529 | 1.00 | 22.16 |
| ATOM | 297 | CA | ILE | A | 576 | 7.091 | 12.673 | 18.748 | 1.00 | 23.75 |
| ATOM | 298 | CB | ILE | A | 576 | 7.403 | 14.171 | 18.924 | 1.00 | 26.26 |
| ATOM | 299 | CG2 | ILE | A | 576 | 8.756 | 14.495 | 18.311 | 1.00 | 26.23 |
| ATOM | 300 | CG1 | ILE | A | 576 | 6.318 | 15.016 | 18.260 | 1.00 | 26.29 |
| ATOM | 301 | CD1 | ILE | A | 576 | 6.476 | 16.494 | 18.490 | 1.00 | 26.83 |
| ATOM | 302 | C | ILE | A | 576 | 8.311 | 11.872 | 19.209 | 1.00 | 22.92 |
| ATOM | 303 | O | ILE | A | 576 | 9.111 | 11.416 | 18.394 | 1.00 | 23.77 |
| ATOM | 304 | N | ARG | A | 577 | 8.447 | 11.703 | 20.521 | 1.00 | 24.95 |
| ATOM | 305 | CA | ARG | A | 577 | 9.575 | 10.965 | 21.077 | 1.00 | 24.47 |
| ATOM | 306 | CB | ARG | A | 577 | 9.560 | 11.050 | 22.609 | 1.00 | 26.95 |
| ATOM | 307 | CG | ARG | A | 577 | 10.652 | 10.234 | 23.294 | 1.00 | 27.15 |
| ATOM | 308 | CD | ARG | A | 577 | 12.031 | 10.561 | 22.731 | 1.00 | 27.06 |
| ATOM | 309 | NE | ARG | A | 577 | 12.398 | 11.956 | 22.957 | 1.00 | 26.65 |
| ATOM | 310 | CZ | ARG | A | 577 | 13.447 | 12.556 | 22.401 | 1.00 | 27.38 |
| ATOM | 311 | NH1 | ARG | A | 577 | 14.247 | 11.888 | 21.578 | 1.00 | 27.39 |
| ATOM | 312 | NH2 | ARG | A | 577 | 13.696 | 13.829 | 22.666 | 1.00 | 26.81 |
| ATOM | 313 | C | ARG | A | 577 | 9.565 | 9.509 | 20.625 | 1.00 | 24.18 |
| ATOM | 314 | O | ARG | A | 577 | 10.619 | 8.913 | 20.416 | 1.00 | 24.97 |
| ATOM | 315 | N | MET | A | 578 | 8.372 | 8.938 | 20.471 | 1.00 | 21.76 |
| ATOM | 316 | CA | MET | A | 578 | 8.247 | 7.552 | 20.032 | 1.00 | 21.44 |
| ATOM | 317 | CB | MET | A | 578 | 6.782 | 7.122 | 20.042 | 1.00 | 25.13 |
| ATOM | 318 | CG | MET | A | 578 | 6.222 | 6.855 | 21.425 | 1.00 | 23.93 |
| ATOM | 319 | SD | MET | A | 578 | 4.483 | 6.363 | 21.351 | 1.00 | 23.95 |
| ATOM | 320 | CE | MET | A | 578 | 3.892 | 6.945 | 22.915 | 1.00 | 24.88 |
| ATOM | 321 | C | MET | A | 578 | 8.820 | 7.370 | 18.630 | 1.00 | 22.27 |
| ATOM | 322 | O | MET | A | 578 | 9.562 | 6.422 | 18.373 | 1.00 | 22.21 |
| ATOM | 323 | N | PHE | A | 579 | 8.462 | 8.270 | 17.722 | 1.00 | 22.29 |
| ATOM | 324 | CA | PHE | A | 579 | 8.960 | 8.194 | 16.355 | 1.00 | 22.58 |
| ATOM | 325 | CB | PHE | A | 579 | 8.317 | 9.267 | 15.474 | 1.00 | 22.36 |
| ATOM | 326 | CG | PHE | A | 579 | 6.970 | 8.885 | 14.925 | 1.00 | 22.77 |
| ATOM | 327 | CD1 | PHE | A | 579 | 5.812 | 9.077 | 15.672 | 1.00 | 22.73 |
| ATOM | 328 | CD2 | PHE | A | 579 | 6.864 | 8.341 | 13.649 | 1.00 | 21.91 |
| ATOM | 329 | CE1 | PHE | A | 579 | 4.560 | 8.733 | 15.151 | 1.00 | 22.92 |
| ATOM | 330 | CE2 | PHE | A | 579 | 5.625 | 7.993 | 13.119 | 1.00 | 22.30 |
| ATOM | 331 | CZ | PHE | A | 579 | 4.469 | 8.190 | 13.871 | 1.00 | 22.31 |
| ATOM | 332 | C | PHE | A | 579 | 10.466 | 8.390 | 16.328 | 1.00 | 24.14 |
| ATOM | 333 | O | PHE | A | 579 | 11.183 | 7.701 | 15.600 | 1.00 | 22.99 |
| ATOM | 334 | N | THR | A | 580 | 10.935 | 9.336 | 17.133 | 1.00 | 24.07 |
| ATOM | 335 | CA | THR | A | 580 | 12.350 | 9.667 | 17.198 | 1.00 | 24.45 |
| ATOM | 336 | CB | THR | A | 580 | 12.567 | 10.901 | 18.085 | 1.00 | 24.15 |
| ATOM | 337 | OG1 | THR | A | 580 | 11.712 | 11.961 | 17.636 | 1.00 | 24.88 |
| ATOM | 338 | CG2 | THR | A | 580 | 14.008 | 11.370 | 18.009 | 1.00 | 24.18 |
| ATOM | 339 | C | THR | A | 580 | 13.239 | 8.535 | 17.691 | 1.00 | 24.89 |
| ATOM | 340 | O | THR | A | 580 | 14.218 | 8.188 | 17.030 | 1.00 | 24.15 |
| ATOM | 341 | N | ASP | A | 581 | 12.903 | 7.955 | 18.840 | 1.00 | 26.03 |
| ATOM | 342 | CA | ASP | A | 581 | 13.703 | 6.870 | 19.402 | 1.00 | 26.27 |
| ATOM | 343 | CB | ASP | A | 581 | 13.395 | 6.710 | 20.894 | 1.00 | 26.96 |
| ATOM | 344 | CG | ASP | A | 581 | 14.029 | 7.808 | 21.733 | 1.00 | 26.96 |
| ATOM | 345 | OD1 | ASP | A | 581 | 14.376 | 8.868 | 21.167 | 1.00 | 26.78 |
| ATOM | 346 | OD2 | ASP | A | 581 | 14.176 | 7.620 | 22.958 | 1.00 | 27.14 |
| ATOM | 347 | C | ASP | A | 581 | 13.578 | 5.534 | 18.676 | 1.00 | 26.95 |
| ATOM | 348 | O | ASP | A | 581 | 14.306 | 4.589 | 18.978 | 1.00 | 27.55 |
| ATOM | 349 | N | LEU | A | 582 | 12.654 | 5.447 | 17.727 | 1.00 | 25.40 |
| ATOM | 350 | CA | LEU | A | 582 | 12.516 | 4.231 | 16.934 | 1.00 | 25.74 |
| ATOM | 351 | CB | LEU | A | 582 | 11.040 | 3.918 | 16.642 | 1.00 | 23.64 |
| ATOM | 352 | CG | LEU | A | 582 | 10.286 | 3.274 | 17.817 | 1.00 | 22.73 |
| ATOM | 353 | CD1 | LEU | A | 582 | 8.801 | 3.146 | 17.484 | 1.00 | 21.47 |
| ATOM | 354 | CD2 | LEU | A | 582 | 10.873 | 1.900 | 18.125 | 1.00 | 22.27 |
| ATOM | 355 | C | LEU | A | 582 | 13.284 | 4.483 | 15.635 | 1.00 | 26.83 |
| ATOM | 356 | O | LEU | A | 582 | 13.259 | 3.669 | 14.710 | 1.00 | 26.36 |
| ATOM | 357 | N | ASN | A | 583 | 13.967 | 5.625 | 15.588 | 1.00 | 30.93 |
| ATOM | 358 | CA | ASN | A | 583 | 14.767 | 6.026 | 14.433 | 1.00 | 32.87 |
| ATOM | 359 | CB | ASN | A | 583 | 15.944 | 5.065 | 14.247 | 1.00 | 52.34 |
| ATOM | 360 | CG | ASN | A | 583 | 17.069 | 5.331 | 15.225 | 1.00 | 53.08 |
| ATOM | 361 | OD1 | ASN | A | 583 | 18.026 | 4.561 | 15.312 | 1.00 | 53.41 |
| ATOM | 362 | ND2 | ASN | A | 583 | 16.967 | 6.432 | 15.962 | 1.00 | 53.31 |
| ATOM | 363 | C | ASN | A | 583 | 13.973 | 6.114 | 13.143 | 1.00 | 31.76 |
| ATOM | 364 | O | ASN | A | 583 | 14.443 | 5.706 | 12.085 | 1.00 | 32.63 |
| ATOM | 365 | N | LEU | A | 584 | 12.769 | 6.665 | 13.235 | 1.00 | 27.18 |
| ATOM | 366 | CA | LEU | A | 584 | 11.904 | 6.813 | 12.073 | 1.00 | 27.03 |
| ATOM | 367 | CB | LEU | A | 584 | 10.455 | 6.521 | 12.468 | 1.00 | 25.42 |
| ATOM | 368 | CG | LEU | A | 584 | 9.877 | 5.107 | 12.377 | 1.00 | 27.73 |
| ATOM | 369 | CD1 | LEU | A | 584 | 10.936 | 4.047 | 12.537 | 1.00 | 26.75 |
| ATOM | 370 | CD2 | LEU | A | 584 | 8.771 | 4.975 | 13.422 | 1.00 | 25.52 |
| ATOM | 371 | C | LEU | A | 584 | 11.971 | 8.208 | 11.468 | 1.00 | 26.54 |
| ATOM | 372 | O | LEU | A | 584 | 11.760 | 8.380 | 10.270 | 1.00 | 27.46 |
| ATOM | 373 | N | VAL | A | 585 | 12.265 | 9.204 | 12.296 | 1.00 | 28.54 |
| ATOM | 374 | CA | VAL | A | 585 | 12.300 | 10.589 | 11.831 | 1.00 | 29.02 |
| ATOM | 375 | CB | VAL | A | 585 | 12.403 | 11.550 | 13.025 | 1.00 | 27.38 |
| ATOM | 376 | CG1 | VAL | A | 585 | 12.404 | 12.993 | 12.545 | 1.00 | 27.75 |
| ATOM | 377 | CG2 | VAL | A | 585 | 11.236 | 11.310 | 13.971 | 1.00 | 27.59 |
| ATOM | 378 | C | VAL | A | 585 | 13.375 | 10.951 | 10.804 | 1.00 | 30.26 |
| ATOM | 379 | O | VAL | A | 585 | 13.059 | 11.379 | 9.693 | 1.00 | 29.85 |
| ATOM | 380 | N | GLN | A | 586 | 14.639 | 10.799 | 11.167 | 1.00 | 36.79 |
| ATOM | 381 | CA | GLN | A | 586 | 15.697 | 11.132 | 10.228 | 1.00 | 38.86 |
| ATOM | 382 | CB | GLN | A | 586 | 17.024 | 11.300 | 10.969 | 1.00 | 56.08 |
| ATOM | 383 | CG | GLN | A | 586 | 16.935 | 12.315 | 12.102 | 1.00 | 56.46 |
| ATOM | 384 | CD | GLN | A | 586 | 18.288 | 12.833 | 12.551 | 1.00 | 56.85 |
| ATOM | 385 | OE1 | GLN | A | 586 | 18.393 | 13.538 | 13.556 | 1.00 | 56.83 |
| ATOM | 386 | NE2 | GLN | A | 586 | 19.332 | 12.494 | 11.801 | 1.00 | 56.71 |
| ATOM | 387 | C | GLN | A | 586 | 15.796 | 10.046 | 9.163 | 1.00 | 38.13 |
| ATOM | 388 | O | GLN | A | 586 | 16.005 | 10.336 | 7.985 | 1.00 | 38.63 |
| ATOM | 389 | N | ASN | A | 587 | 15.615 | 8.799 | 9.586 | 1.00 | 34.73 |
| ATOM | 390 | CA | ASN | A | 587 | 15.665 | 7.652 | 8.685 | 1.00 | 34.34 |
| ATOM | 391 | CB | ASN | A | 587 | 15.120 | 6.420 | 9.399 | 1.00 | 37.16 |
| ATOM | 392 | CG | ASN | A | 587 | 15.340 | 5.140 | 8.620 | 1.00 | 36.75 |
| ATOM | 393 | OD1 | ASN | A | 587 | 15.392 | 5.141 | 7.389 | 1.00 | 36.63 |
| ATOM | 394 | ND2 | ASN | A | 587 | 15.450 | 4.031 | 9.341 | 1.00 | 37.09 |
| ATOM | 395 | C | ASN | A | 587 | 14.834 | 7.911 | 7.430 | 1.00 | 34.66 |
| ATOM | 396 | O | ASN | A | 587 | 15.294 | 7.691 | 6.307 | 1.00 | 34.46 |
| ATOM | 397 | N | PHE | A | 588 | 13.600 | 8.372 | 7.628 | 1.00 | 30.54 |
| ATOM | 398 | CA | PHE | A | 588 | 12.711 | 8.654 | 6.511 | 1.00 | 30.38 |
| ATOM | 399 | CB | PHE | A | 588 | 11.366 | 7.964 | 6.734 | 1.00 | 24.15 |
| ATOM | 400 | CG | PHE | A | 588 | 11.487 | 6.479 | 6.910 | 1.00 | 23.77 |
| ATOM | 401 | CD1 | PHE | A | 588 | 11.369 | 5.896 | 8.169 | 1.00 | 24.08 |
| ATOM | 402 | CD2 | PHE | A | 588 | 11.762 | 5.663 | 5.820 | 1.00 | 23.24 |
| ATOM | 403 | CE1 | PHE | A | 588 | 11.526 | 4.515 | 8.336 | 1.00 | 24.41 |
| ATOM | 404 | CE2 | PHE | A | 588 | 11.921 | 4.285 | 5.969 | 1.00 | 23.43 |
| ATOM | 405 | CZ | PHE | A | 588 | 11.804 | 3.707 | 7.228 | 1.00 | 23.76 |
| ATOM | 406 | C | PHE | A | 588 | 12.535 | 10.151 | 6.304 | 1.00 | 30.36 |
| ATOM | 407 | O | PHE | A | 588 | 11.595 | 10.599 | 5.649 | 1.00 | 30.79 |
| ATOM | 408 | N | GLN | A | 589 | 13.462 | 10.912 | 6.873 | 1.00 | 35.42 |
| ATOM | 409 | CA | GLN | A | 589 | 13.489 | 12.366 | 6.750 | 1.00 | 36.58 |
| ATOM | 410 | CB | GLN | A | 589 | 14.167 | 12.739 | 5.433 | 1.00 | 49.72 |
| ATOM | 411 | CG | GLN | A | 589 | 15.501 | 12.043 | 5.243 | 1.00 | 49.96 |
| ATOM | 412 | CD | GLN | A | 589 | 15.680 | 11.505 | 3.839 | 1.00 | 50.27 |
| ATOM | 413 | OE1 | GLN | A | 589 | 14.788 | 10.852 | 3.296 | 1.00 | 50.16 |
| ATOM | 414 | NE2 | GLN | A | 589 | 16.839 | 11.762 | 3.249 | 1.00 | 50.04 |
| ATOM | 415 | C | GLN | A | 589 | 12.133 | 13.055 | 6.846 | 1.00 | 36.29 |
| ATOM | 416 | O | GLN | A | 589 | 11.648 | 13.655 | 5.882 | 1.00 | 36.29 |
| ATOM | 417 | N | MET | A | 590 | 11.524 | 12.969 | 8.021 | 1.00 | 36.14 |
| ATOM | 418 | CA | MET | A | 590 | 10.240 | 13.608 | 8.258 | 1.00 | 35.67 |
| ATOM | 419 | CB | MET | A | 590 | 9.459 | 12.847 | 9.331 | 1.00 | 32.44 |
| ATOM | 420 | CG | MET | A | 590 | 9.055 | 11.431 | 8.966 | 1.00 | 31.88 |
| ATOM | 421 | SD | MET | A | 590 | 8.254 | 10.607 | 10.373 | 1.00 | 31.19 |
| ATOM | 422 | CE | MET | A | 590 | 6.882 | 11.641 | 10.615 | 1.00 | 32.88 |
| ATOM | 423 | C | MET | A | 590 | 10.515 | 15.022 | 8.759 | 1.00 | 35.35 |
| ATOM | 424 | O | MET | A | 590 | 11.351 | 15.209 | 9.641 | 1.00 | 35.80 |
| ATOM | 425 | N | LYS | A | 591 | 9.839 | 16.014 | 8.191 | 1.00 | 35.07 |
| ATOM | 426 | CA | LYS | A | 591 | 10.016 | 17.389 | 8.648 | 1.00 | 34.67 |
| ATOM | 427 | CB | LYS | A | 591 | 9.427 | 18.381 | 7.641 | 1.00 | 46.77 |
| ATOM | 428 | CG | LYS | A | 591 | 10.350 | 18.721 | 6.480 | 1.00 | 47.35 |
| ATOM | 429 | CD | LYS | A | 591 | 10.650 | 17.509 | 5.612 | 1.00 | 47.48 |
| ATOM | 430 | CE | LYS | A | 591 | 11.738 | 17.813 | 4.587 | 1.00 | 47.57 |
| ATOM | 431 | NZ | LYS | A | 591 | 11.398 | 18.958 | 3.694 | 1.00 | 47.52 |
| ATOM | 432 | C | LYS | A | 591 | 9.274 | 17.492 | 9.976 | 1.00 | 33.93 |
| ATOM | 433 | O | LYS | A | 591 | 8.132 | 17.049 | 10.081 | 1.00 | 34.06 |
| ATOM | 434 | N | HIS | A | 592 | 9.926 | 18.068 | 10.983 | 1.00 | 27.75 |
| ATOM | 435 | CA | HIS | A | 592 | 9.332 | 18.205 | 12.310 | 1.00 | 26.86 |
| ATOM | 436 | CB | HIS | A | 592 | 10.227 | 19.071 | 13.208 | 1.00 | 27.82 |
| ATOM | 437 | CG | HIS | A | 592 | 9.770 | 19.139 | 14.632 | 1.00 | 26.72 |
| ATOM | 438 | CD2 | HIS | A | 592 | 9.317 | 20.178 | 15.374 | 1.00 | 27.57 |
| ATOM | 439 | ND1 | HIS | A | 592 | 9.755 | 18.040 | 15.464 | 1.00 | 27.40 |
| ATOM | 440 | CE1 | HIS | A | 592 | 9.314 | 18.399 | 16.657 | 1.00 | 27.96 |
| ATOM | 441 | NE2 | HIS | A | 592 | 9.041 | 19.692 | 16.628 | 1.00 | 27.67 |
| ATOM | 442 | C | HIS | A | 592 | 7.927 | 18.796 | 12.277 | 1.00 | 28.09 |
| ATOM | 443 | O | HIS | A | 592 | 7.021 | 18.288 | 12.933 | 1.00 | 26.68 |
| ATOM | 444 | N | GLU | A | 593 | 7.749 | 19.865 | 11.508 | 1.00 | 32.89 |
| ATOM | 445 | CA | GLU | A | 593 | 6.456 | 20.534 | 11.405 | 1.00 | 34.01 |
| ATOM | 446 | CB | GLU | A | 593 | 6.572 | 21.771 | 10.510 | 1.00 | 53.17 |
| ATOM | 447 | CG | GLU | A | 593 | 7.483 | 22.870 | 11.051 | 1.00 | 53.98 |
| ATOM | 448 | CD | GLU | A | 593 | 8.914 | 22.403 | 11.272 | 1.00 | 54.08 |
| ATOM | 449 | OE1 | GLU | A | 593 | 9.519 | 21.853 | 10.326 | 1.00 | 53.79 |
| ATOM | 450 | OE2 | GLU | A | 593 | 9.434 | 22.591 | 12.394 | 1.00 | 53.97 |
| ATOM | 451 | C | GLU | A | 593 | 5.370 | 19.613 | 10.856 | 1.00 | 33.01 |
| ATOM | 452 | O | GLU | A | 593 | 4.230 | 19.634 | 11.319 | 1.00 | 32.84 |
| ATOM | 453 | N | VAL | A | 594 | 5.730 | 18.808 | 9.864 | 1.00 | 27.04 |
| ATOM | 454 | CA | VAL | A | 594 | 4.783 | 17.886 | 9.249 | 1.00 | 25.85 |
| ATOM | 455 | CB | VAL | A | 594 | 5.382 | 17.279 | 7.962 | 1.00 | 24.72 |
| ATOM | 456 | CG1 | VAL | A | 594 | 4.446 | 16.233 | 7.381 | 1.00 | 24.57 |
| ATOM | 457 | CG2 | VAL | A | 594 | 5.631 | 18.383 | 6.945 | 1.00 | 24.02 |
| ATOM | 458 | C | VAL | A | 594 | 4.386 | 16.771 | 10.215 | 1.00 | 26.36 |
| ATOM | 459 | O | VAL | A | 594 | 3.214 | 16.386 | 10.283 | 1.00 | 25.05 |
| ATOM | 460 | N | LEU | A | 595 | 5.356 | 16.246 | 10.956 | 1.00 | 24.15 |
| ATOM | 461 | CA | LEU | A | 595 | 5.062 | 15.187 | 11.918 | 1.00 | 25.41 |
| ATOM | 462 | CB | LEU | A | 595 | 6.352 | 14.633 | 12.536 | 1.00 | 24.61 |
| ATOM | 463 | CG | LEU | A | 595 | 6.204 | 13.640 | 13.696 | 1.00 | 25.86 |
| ATOM | 464 | CD1 | LEU | A | 595 | 5.345 | 12.453 | 13.272 | 1.00 | 24.75 |
| ATOM | 465 | CD2 | LEU | A | 595 | 7.577 | 13.163 | 14.141 | 1.00 | 25.05 |
| ATOM | 466 | C | LEU | A | 595 | 4.148 | 15.745 | 13.009 | 1.00 | 25.02 |
| ATOM | 467 | O | LEU | A | 595 | 3.197 | 15.088 | 13.435 | 1.00 | 24.20 |
| ATOM | 468 | N | CYS | A | 596 | 4.426 | 16.970 | 13.451 | 1.00 | 24.36 |
| ATOM | 469 | CA | CYS | A | 596 | 3.603 | 17.588 | 14.479 | 1.00 | 24.03 |
| ATOM | 470 | CB | CYS | A | 596 | 4.195 | 18.931 | 14.907 | 1.00 | 27.15 |
| ATOM | 471 | SG | CYS | A | 596 | 5.699 | 18.771 | 15.892 | 1.00 | 26.30 |
| ATOM | 472 | C | CYS | A | 596 | 2.177 | 17.789 | 13.987 | 1.00 | 24.31 |
| ATOM | 473 | O | CYS | A | 596 | 1.222 | 17.537 | 14.721 | 1.00 | 24.29 |
| ATOM | 474 | N | ARG | A | 597 | 2.032 | 18.233 | 12.742 | 1.00 | 22.43 |
| ATOM | 475 | CA | ARG | A | 597 | 0.703 | 18.460 | 12.194 | 1.00 | 22.27 |
| ATOM | 476 | CB | ARG | A | 597 | 0.777 | 19.177 | 10.841 | 1.00 | 37.36 |
| ATOM | 477 | CG | ARG | A | 597 | −0.597 | 19.576 | 10.315 | 1.00 | 38.44 |
| ATOM | 478 | CD | ARG | A | 597 | −0.541 | 20.392 | 9.032 | 1.00 | 38.82 |
| ATOM | 479 | NE | ARG | A | 597 | 0.033 | 21.719 | 9.233 | 1.00 | 38.78 |
| ATOM | 480 | CZ | ARG | A | 597 | 1.286 | 22.043 | 8.936 | 1.00 | 38.71 |
| ATOM | 481 | NH1 | ARG | A | 597 | 2.101 | 21.133 | 8.421 | 1.00 | 38.58 |
| ATOM | 482 | NH2 | ARG | A | 597 | 1.721 | 23.278 | 9.149 | 1.00 | 38.90 |
| ATOM | 483 | C | ARG | A | 597 | −0.042 | 17.140 | 12.042 | 1.00 | 21.87 |
| ATOM | 484 | O | ARG | A | 597 | −1.231 | 17.049 | 12.347 | 1.00 | 22.25 |
| ATOM | 485 | N | TRP | A | 598 | 0.661 | 16.114 | 11.578 | 1.00 | 22.62 |
| ATOM | 486 | CA | TRP | A | 598 | 0.039 | 14.802 | 11.396 | 1.00 | 20.68 |
| ATOM | 487 | CB | TRP | A | 598 | 1.048 | 13.826 | 10.788 | 1.00 | 21.19 |
| ATOM | 488 | CG | TRP | A | 598 | 0.506 | 12.443 | 10.641 | 1.00 | 20.61 |
| ATOM | 489 | CD2 | TRP | A | 598 | 0.680 | 11.358 | 11.554 | 1.00 | 21.45 |
| ATOM | 490 | CE2 | TRP | A | 598 | −0.059 | 10.262 | 11.048 | 1.00 | 20.54 |
| ATOM | 491 | CE3 | TRP | A | 598 | 1.386 | 11.201 | 12.755 | 1.00 | 20.69 |
| ATOM | 492 | CD1 | TRP | A | 598 | −0.304 | 11.982 | 9.644 | 1.00 | 21.12 |
| ATOM | 493 | NE1 | TRP | A | 598 | −0.648 | 10.671 | 9.882 | 1.00 | 21.75 |
| ATOM | 494 | CZ2 | TRP | A | 598 | −0.111 | 9.028 | 11.701 | 1.00 | 20.03 |
| ATOM | 495 | CZ3 | TRP | A | 598 | 1.334 | 9.974 | 13.406 | 1.00 | 21.23 |
| ATOM | 496 | CH2 | TRP | A | 598 | 0.588 | 8.901 | 12.875 | 1.00 | 21.56 |
| ATOM | 497 | C | TRP | A | 598 | −0.461 | 14.256 | 12.734 | 1.00 | 20.83 |
| ATOM | 498 | O | TRP | A | 598 | −1.575 | 13.743 | 12.833 | 1.00 | 20.85 |
| ATOM | 499 | N | ILE | A | 599 | 0.371 | 14.362 | 13.762 | 1.00 | 18.38 |
| ATOM | 500 | CA | ILE | A | 599 | −0.001 | 13.881 | 15.081 | 1.00 | 17.01 |
| ATOM | 501 | CB | ILE | A | 599 | 1.138 | 14.106 | 16.103 | 1.00 | 18.07 |
| ATOM | 502 | CG2 | ILE | A | 599 | 0.625 | 13.881 | 17.516 | 1.00 | 18.87 |
| ATOM | 503 | CG1 | ILE | A | 599 | 2.304 | 13.158 | 15.794 | 1.00 | 18.94 |
| ATOM | 504 | CD1 | ILE | A | 599 | 3.523 | 13.367 | 16.669 | 1.00 | 18.33 |
| ATOM | 505 | C | ILE | A | 599 | −1.263 | 14.596 | 15.554 | 1.00 | 18.40 |
| ATOM | 506 | O | ILE | A | 599 | −2.184 | 13.969 | 16.081 | 1.00 | 16.74 |
| ATOM | 507 | N | LEU | A | 600 | −1.311 | 15.912 | 15.371 | 1.00 | 21.06 |
| ATOM | 508 | CA | LEU | A | 600 | −2.486 | 16.657 | 15.802 | 1.00 | 21.13 |
| ATOM | 509 | CB | LEU | A | 600 | −2.202 | 18.162 | 15.761 | 1.00 | 21.76 |
| ATOM | 510 | CG | LEU | A | 600 | −1.149 | 18.582 | 16.788 | 1.00 | 21.76 |
| ATOM | 511 | CD1 | LEU | A | 600 | −0.811 | 20.053 | 16.597 | 1.00 | 21.85 |
| ATOM | 512 | CD2 | LEU | A | 600 | −1.672 | 18.324 | 18.201 | 1.00 | 22.51 |
| ATOM | 513 | C | LEU | A | 600 | −3.728 | 16.325 | 14.979 | 1.00 | 20.73 |
| ATOM | 514 | O | LEU | A | 600 | −4.841 | 16.351 | 15.501 | 1.00 | 21.49 |
| ATOM | 515 | N | SER | A | 601 | −3.540 | 16.032 | 13.694 | 1.00 | 21.08 |
| ATOM | 516 | CA | SER | A | 601 | −4.665 | 15.674 | 12.833 | 1.00 | 20.75 |
| ATOM | 517 | CB | SER | A | 601 | −4.225 | 15.579 | 11.372 | 1.00 | 22.55 |
| ATOM | 518 | OG | SER | A | 601 | −4.008 | 16.869 | 10.822 | 1.00 | 23.14 |
| ATOM | 519 | C | SER | A | 601 | −5.228 | 14.333 | 13.287 | 1.00 | 20.19 |
| ATOM | 520 | O | SER | A | 601 | −6.438 | 14.125 | 13.275 | 1.00 | 20.13 |
| ATOM | 521 | N | VAL | A | 602 | −4.340 | 13.424 | 13.679 | 1.00 | 21.43 |
| ATOM | 522 | CA | VAL | A | 602 | −4.773 | 12.116 | 14.168 | 1.00 | 21.85 |
| ATOM | 523 | CB | VAL | A | 602 | −3.564 | 11.204 | 14.471 | 1.00 | 17.69 |
| ATOM | 524 | CG1 | VAL | A | 602 | −4.018 | 9.928 | 15.198 | 1.00 | 18.88 |
| ATOM | 525 | CG2 | VAL | A | 602 | −2.867 | 10.845 | 13.174 | 1.00 | 19.25 |
| ATOM | 526 | C | VAL | A | 602 | −5.581 | 12.312 | 15.445 | 1.00 | 21.58 |
| ATOM | 527 | O | VAL | A | 602 | −6.705 | 11.823 | 15.566 | 1.00 | 20.63 |
| ATOM | 528 | N | LYS | A | 603 | −5.011 | 13.052 | 16.394 | 1.00 | 20.30 |
| ATOM | 529 | CA | LYS | A | 603 | −5.697 | 13.303 | 17.655 | 1.00 | 19.93 |
| ATOM | 530 | CB | LYS | A | 603 | −4.833 | 14.186 | 18.567 | 1.00 | 29.59 |
| ATOM | 531 | CG | LYS | A | 603 | −5.553 | 14.663 | 19.829 | 1.00 | 29.47 |
| ATOM | 532 | CD | LYS | A | 603 | −4.587 | 15.206 | 20.882 | 1.00 | 30.01 |
| ATOM | 533 | CE | LYS | A | 603 | −3.846 | 16.449 | 20.416 | 1.00 | 30.55 |
| ATOM | 534 | NZ | LYS | A | 603 | −2.899 | 16.934 | 21.464 | 1.00 | 31.23 |
| ATOM | 535 | C | LYS | A | 603 | −7.052 | 13.968 | 17.430 | 1.00 | 19.43 |
| ATOM | 536 | O | LYS | A | 603 | −8.034 | 13.636 | 18.095 | 1.00 | 20.76 |
| ATOM | 537 | N | LYS | A | 604 | −7.100 | 14.894 | 16.481 | 1.00 | 19.98 |
| ATOM | 538 | CA | LYS | A | 604 | −8.325 | 15.629 | 16.174 | 1.00 | 21.57 |
| ATOM | 539 | CB | LYS | A | 604 | −8.024 | 16.775 | 15.207 | 1.00 | 38.76 |
| ATOM | 540 | CG | LYS | A | 604 | −9.262 | 17.531 | 14.741 | 1.00 | 39.38 |
| ATOM | 541 | CD | LYS | A | 604 | −8.910 | 18.610 | 13.732 | 1.00 | 39.39 |
| ATOM | 542 | CE | LYS | A | 604 | −10.158 | 19.340 | 13.255 | 1.00 | 39.74 |
| ATOM | 543 | NZ | LYS | A | 604 | −9.843 | 20.385 | 12.242 | 1.00 | 39.72 |
| ATOM | 544 | C | LYS | A | 604 | −9.413 | 14.754 | 15.579 | 1.00 | 20.90 |
| ATOM | 545 | O | LYS | A | 604 | −10.609 | 15.039 | 15.742 | 1.00 | 21.11 |
| ATOM | 546 | N | ASN | A | 605 | −9.009 | 13.688 | 14.892 | 1.00 | 21.37 |
| ATOM | 547 | CA | ASN | A | 605 | −9.987 | 12.812 | 14.273 | 1.00 | 21.04 |
| ATOM | 548 | CB | ASN | A | 605 | −9.486 | 12.315 | 12.920 | 1.00 | 27.07 |
| ATOM | 549 | CG | ASN | A | 605 | −9.635 | 13.366 | 11.836 | 1.00 | 27.98 |
| ATOM | 550 | OD1 | ASN | A | 605 | −8.775 | 14.234 | 11.666 | 1.00 | 27.85 |
| ATOM | 551 | ND2 | ASN | A | 605 | −10.745 | 13.308 | 11.112 | 1.00 | 27.52 |
| ATOM | 552 | C | ASN | A | 605 | −10.506 | 11.662 | 15.116 | 1.00 | 21.57 |
| ATOM | 553 | O | ASN | A | 605 | −11.072 | 10.702 | 14.590 | 1.00 | 23.07 |
| ATOM | 554 | N | TYR | A | 606 | −10.292 | 11.754 | 16.422 | 1.00 | 20.07 |
| ATOM | 555 | CA | TYR | A | 606 | −10.853 | 10.792 | 17.349 | 1.00 | 20.46 |
| ATOM | 556 | CB | TYR | A | 606 | −9.858 | 10.374 | 18.437 | 1.00 | 18.79 |
| ATOM | 557 | CG | TYR | A | 606 | −8.993 | 9.216 | 17.992 | 1.00 | 18.19 |
| ATOM | 558 | CD1 | TYR | A | 606 | −7.745 | 9.430 | 17.409 | 1.00 | 19.08 |
| ATOM | 559 | CE1 | TYR | A | 606 | −6.989 | 8.362 | 16.913 | 1.00 | 18.94 |
| ATOM | 560 | CD2 | TYR | A | 606 | −9.467 | 7.909 | 18.074 | 1.00 | 18.23 |
| ATOM | 561 | CE2 | TYR | A | 606 | −8.722 | 6.840 | 17.578 | 1.00 | 17.63 |
| ATOM | 562 | CZ | TYR | A | 606 | −7.485 | 7.077 | 16.999 | 1.00 | 18.23 |
| ATOM | 563 | OH | TYR | A | 606 | −6.747 | 6.029 | 16.489 | 1.00 | 15.86 |
| ATOM | 564 | C | TYR | A | 606 | −12.015 | 11.580 | 17.947 | 1.00 | 21.76 |
| ATOM | 565 | O | TYR | A | 606 | −11.975 | 12.819 | 18.007 | 1.00 | 20.70 |
| ATOM | 566 | N | ARG | A | 607 | −13.060 | 10.882 | 18.365 | 1.00 | 19.57 |
| ATOM | 567 | CA | ARG | A | 607 | −14.223 | 11.562 | 18.931 | 1.00 | 20.12 |
| ATOM | 568 | CB | ARG | A | 607 | −15.497 | 10.791 | 18.575 | 1.00 | 23.54 |
| ATOM | 569 | CG | ARG | A | 607 | −15.679 | 10.605 | 17.074 | 1.00 | 23.38 |
| ATOM | 570 | CD | ARG | A | 607 | −17.034 | 10.010 | 16.715 | 1.00 | 23.37 |
| ATOM | 571 | NE | ARG | A | 607 | −17.142 | 9.734 | 15.282 | 1.00 | 24.11 |
| ATOM | 572 | CZ | ARG | A | 607 | −18.274 | 9.408 | 14.665 | 1.00 | 24.11 |
| ATOM | 573 | NH1 | ARG | A | 607 | −19.405 | 9.321 | 15.355 | 1.00 | 24.42 |
| ATOM | 574 | NH2 | ARG | A | 607 | −18.274 | 9.163 | 13.362 | 1.00 | 25.10 |
| ATOM | 575 | C | ARG | A | 607 | −14.037 | 11.647 | 20.438 | 1.00 | 20.33 |
| ATOM | 576 | O | ARG | A | 607 | −14.168 | 10.648 | 21.150 | 1.00 | 20.09 |
| ATOM | 577 | N | LYS | A | 608 | −13.736 | 12.847 | 20.926 | 1.00 | 25.24 |
| ATOM | 578 | CA | LYS | A | 608 | −13.484 | 13.033 | 22.349 | 1.00 | 25.95 |
| ATOM | 579 | CB | LYS | A | 608 | −13.108 | 14.491 | 22.636 | 1.00 | 41.13 |
| ATOM | 580 | CG | LYS | A | 608 | −14.175 | 15.511 | 22.294 | 1.00 | 41.73 |
| ATOM | 581 | CD | LYS | A | 608 | −13.670 | 16.916 | 22.603 | 1.00 | 41.87 |
| ATOM | 582 | CE | LYS | A | 608 | −14.750 | 17.972 | 22.413 | 1.00 | 41.79 |
| ATOM | 583 | NZ | LYS | A | 608 | −14.231 | 19.340 | 22.703 | 1.00 | 41.73 |
| ATOM | 584 | C | LYS | A | 608 | −14.625 | 12.599 | 23.254 | 1.00 | 26.44 |
| ATOM | 585 | O | LYS | A | 608 | −14.397 | 12.225 | 24.405 | 1.00 | 25.96 |
| ATOM | 586 | N | ASN | A | 609 | −15.847 | 12.626 | 22.732 | 1.00 | 27.23 |
| ATOM | 587 | CA | ASN | A | 609 | −17.005 | 12.248 | 23.522 | 1.00 | 27.32 |
| ATOM | 588 | CB | ASN | A | 609 | −18.212 | 13.066 | 23.071 | 1.00 | 36.30 |
| ATOM | 589 | CG | ASN | A | 609 | −17.995 | 14.554 | 23.282 | 1.00 | 36.84 |
| ATOM | 590 | OD1 | ASN | A | 609 | −17.809 | 15.008 | 24.411 | 1.00 | 37.02 |
| ATOM | 591 | ND2 | ASN | A | 609 | −17.998 | 15.316 | 22.199 | 1.00 | 37.30 |
| ATOM | 592 | C | ASN | A | 609 | −17.317 | 10.758 | 23.541 | 1.00 | 26.80 |
| ATOM | 593 | O | ASN | A | 609 | −18.330 | 10.332 | 24.098 | 1.00 | 27.72 |
| ATOM | 594 | N | VAL | A | 610 | −16.451 | 9.964 | 22.920 | 1.00 | 21.65 |
| ATOM | 595 | CA | VAL | A | 610 | −16.616 | 8.512 | 22.946 | 1.00 | 20.92 |
| ATOM | 596 | CB | VAL | A | 610 | −16.097 | 7.862 | 21.645 | 1.00 | 20.35 |
| ATOM | 597 | CG1 | VAL | A | 610 | −15.897 | 6.370 | 21.846 | 1.00 | 20.76 |
| ATOM | 598 | CG2 | VAL | A | 610 | −17.120 | 8.086 | 20.522 | 1.00 | 18.26 |
| ATOM | 599 | C | VAL | A | 610 | −15.780 | 8.109 | 24.171 | 1.00 | 19.89 |
| ATOM | 600 | O | VAL | A | 610 | −14.590 | 8.414 | 24.254 | 1.00 | 20.96 |
| ATOM | 601 | N | ALA | A | 611 | −16.423 | 7.450 | 25.130 | 1.00 | 19.80 |
| ATOM | 602 | CA | ALA | A | 611 | −15.791 | 7.078 | 26.397 | 1.00 | 19.90 |
| ATOM | 603 | CB | ALA | A | 611 | −16.780 | 6.287 | 27.251 | 1.00 | 20.34 |
| ATOM | 604 | C | ALA | A | 611 | −14.448 | 6.363 | 26.383 | 1.00 | 19.34 |
| ATOM | 605 | O | ALA | A | 611 | −13.549 | 6.731 | 27.138 | 1.00 | 20.80 |
| ATOM | 606 | N | TYR | A | 612 | −14.307 | 5.343 | 25.542 | 1.00 | 18.10 |
| ATOM | 607 | CA | TYR | A | 612 | −13.060 | 4.599 | 25.512 | 1.00 | 15.69 |
| ATOM | 608 | CB | TYR | A | 612 | −13.321 | 3.134 | 25.873 | 1.00 | 20.49 |
| ATOM | 609 | CG | TYR | A | 612 | −12.107 | 2.251 | 25.709 | 1.00 | 20.53 |
| ATOM | 610 | CD1 | TYR | A | 612 | −12.047 | 1.304 | 24.685 | 1.00 | 21.50 |
| ATOM | 611 | CE1 | TYR | A | 612 | −10.932 | 0.484 | 24.536 | 1.00 | 20.27 |
| ATOM | 612 | CD2 | TYR | A | 612 | −11.020 | 2.359 | 26.577 | 1.00 | 20.70 |
| ATOM | 613 | CE2 | TYR | A | 612 | −9.906 | 1.546 | 26.434 | 1.00 | 19.44 |
| ATOM | 614 | CZ | TYR | A | 612 | −9.870 | 0.611 | 25.414 | 1.00 | 20.76 |
| ATOM | 615 | OH | TYR | A | 612 | −8.769 | −0.205 | 25.284 | 1.00 | 20.43 |
| ATOM | 616 | C | TYR | A | 612 | −12.264 | 4.682 | 24.218 | 1.00 | 14.59 |
| ATOM | 617 | O | TYR | A | 612 | −11.064 | 4.921 | 24.268 | 1.00 | 15.21 |
| ATOM | 618 | N | HIS | A | 613 | −12.915 | 4.500 | 23.069 | 1.00 | 16.35 |
| ATOM | 619 | CA | HIS | A | 613 | −12.197 | 4.553 | 21.793 | 1.00 | 15.66 |
| ATOM | 620 | CB | HIS | A | 613 | −12.942 | 3.763 | 20.705 | 1.00 | 19.05 |
| ATOM | 621 | CG | HIS | A | 613 | −12.971 | 2.286 | 20.941 | 1.00 | 21.28 |
| ATOM | 622 | CD2 | HIS | A | 613 | −12.010 | 1.341 | 20.797 | 1.00 | 20.01 |
| ATOM | 623 | ND1 | HIS | A | 613 | −14.087 | 1.628 | 21.408 | 1.00 | 19.61 |
| ATOM | 624 | CE1 | HIS | A | 613 | −13.816 | 0.342 | 21.541 | 1.00 | 20.40 |
| ATOM | 625 | NE2 | HIS | A | 613 | −12.562 | 0.140 | 21.176 | 1.00 | 20.73 |
| ATOM | 626 | C | HIS | A | 613 | −11.998 | 5.989 | 21.330 | 1.00 | 15.17 |
| ATOM | 627 | O | HIS | A | 613 | −12.633 | 6.451 | 20.376 | 1.00 | 17.00 |
| ATOM | 628 | N | ASN | A | 614 | −11.101 | 6.682 | 22.020 | 1.00 | 18.64 |
| ATOM | 629 | CA | ASN | A | 614 | −10.784 | 8.068 | 21.725 | 1.00 | 17.28 |
| ATOM | 630 | CB | ASN | A | 614 | −11.344 | 8.977 | 22.827 | 1.00 | 21.97 |
| ATOM | 631 | CG | ASN | A | 614 | −10.939 | 8.530 | 24.219 | 1.00 | 21.21 |
| ATOM | 632 | OD1 | ASN | A | 614 | −9.752 | 8.417 | 24.526 | 1.00 | 22.66 |
| ATOM | 633 | ND2 | ASN | A | 614 | −11.928 | 8.277 | 25.074 | 1.00 | 21.32 |
| ATOM | 634 | C | ASN | A | 614 | −9.265 | 8.207 | 21.628 | 1.00 | 18.30 |
| ATOM | 635 | O | ASN | A | 614 | −8.543 | 7.210 | 21.671 | 1.00 | 17.11 |
| ATOM | 636 | N | TRP | A | 615 | −8.773 | 9.434 | 21.504 | 1.00 | 20.23 |
| ATOM | 637 | CA | TRP | A | 615 | −7.335 | 9.650 | 21.388 | 1.00 | 20.84 |
| ATOM | 638 | CB | TRP | A | 615 | −7.020 | 11.152 | 21.346 | 1.00 | 18.84 |
| ATOM | 639 | CG | TRP | A | 615 | −5.567 | 11.469 | 21.610 | 1.00 | 20.81 |
| ATOM | 640 | CD2 | TRP | A | 615 | −4.446 | 11.146 | 20.775 | 1.00 | 20.87 |
| ATOM | 641 | CE2 | TRP | A | 615 | −3.287 | 11.632 | 21.427 | 1.00 | 20.24 |
| ATOM | 642 | CE3 | TRP | A | 615 | −4.307 | 10.493 | 19.542 | 1.00 | 20.69 |
| ATOM | 643 | CD1 | TRP | A | 615 | −5.055 | 12.115 | 22.700 | 1.00 | 19.88 |
| ATOM | 644 | NE1 | TRP | A | 615 | −3.689 | 12.217 | 22.599 | 1.00 | 19.37 |
| ATOM | 645 | CZ2 | TRP | A | 615 | −2.007 | 11.488 | 20.886 | 1.00 | 21.34 |
| ATOM | 646 | CZ3 | TRP | A | 615 | −3.031 | 10.346 | 19.004 | 1.00 | 21.28 |
| ATOM | 647 | CH2 | TRP | A | 615 | −1.897 | 10.845 | 19.679 | 1.00 | 21.14 |
| ATOM | 648 | C | TRP | A | 615 | −6.457 | 8.993 | 22.453 | 1.00 | 19.80 |
| ATOM | 649 | O | TRP | A | 615 | −5.383 | 8.479 | 22.133 | 1.00 | 19.45 |
| ATOM | 650 | N | ARG | A | 616 | −6.888 | 9.010 | 23.713 | 1.00 | 19.15 |
| ATOM | 651 | CA | ARG | A | 616 | −6.063 | 8.424 | 24.764 | 1.00 | 18.73 |
| ATOM | 652 | CB | ARG | A | 616 | −6.658 | 8.707 | 26.146 | 1.00 | 20.84 |
| ATOM | 653 | CG | ARG | A | 616 | −6.628 | 10.197 | 26.516 | 1.00 | 20.98 |
| ATOM | 654 | CD | ARG | A | 616 | −5.208 | 10.754 | 26.511 | 1.00 | 21.78 |
| ATOM | 655 | NE | ARG | A | 616 | −5.185 | 12.195 | 26.772 | 1.00 | 22.05 |
| ATOM | 656 | CZ | ARG | A | 616 | −4.519 | 12.760 | 27.773 | 1.00 | 22.62 |
| ATOM | 657 | NH1 | ARG | A | 616 | −3.820 | 12.012 | 28.610 | 1.00 | 22.11 |
| ATOM | 658 | NH2 | ARG | A | 616 | −4.558 | 14.074 | 27.936 | 1.00 | 22.34 |
| ATOM | 659 | C | ARG | A | 616 | −5.869 | 6.922 | 24.559 | 1.00 | 18.23 |
| ATOM | 660 | O | ARG | A | 616 | −4.799 | 6.388 | 24.857 | 1.00 | 18.43 |
| ATOM | 661 | N | HIS | A | 617 | −6.889 | 6.246 | 24.041 | 1.00 | 17.23 |
| ATOM | 662 | CA | HIS | A | 617 | −6.754 | 4.812 | 23.796 | 1.00 | 17.41 |
| ATOM | 663 | CB | HIS | A | 617 | −8.095 | 4.194 | 23.403 | 1.00 | 16.86 |
| ATOM | 664 | CG | HIS | A | 617 | −7.964 | 2.830 | 22.798 | 1.00 | 13.76 |
| ATOM | 665 | CD2 | HIS | A | 617 | −8.291 | 2.369 | 21.569 | 1.00 | 17.85 |
| ATOM | 666 | ND1 | HIS | A | 617 | −7.384 | 1.772 | 23.465 | 1.00 | 17.12 |
| ATOM | 667 | CE1 | HIS | A | 617 | −7.357 | 0.716 | 22.670 | 1.00 | 15.55 |
| ATOM | 668 | NE2 | HIS | A | 617 | −7.899 | 1.052 | 21.515 | 1.00 | 16.82 |
| ATOM | 669 | C | HIS | A | 617 | −5.737 | 4.566 | 22.682 | 1.00 | 17.79 |
| ATOM | 670 | O | HIS | A | 617 | −4.911 | 3.643 | 22.770 | 1.00 | 17.66 |
| ATOM | 671 | N | ALA | A | 618 | −5.807 | 5.384 | 21.635 | 1.00 | 16.69 |
| ATOM | 672 | CA | ALA | A | 618 | −4.897 | 5.268 | 20.499 | 1.00 | 17.60 |
| ATOM | 673 | CB | ALA | A | 618 | −5.268 | 6.270 | 19.416 | 1.00 | 19.06 |
| ATOM | 674 | C | ALA | A | 618 | −3.478 | 5.517 | 20.973 | 1.00 | 18.60 |
| ATOM | 675 | O | ALA | A | 618 | −2.553 | 4.777 | 20.642 | 1.00 | 16.65 |
| ATOM | 676 | N | PHE | A | 619 | −3.327 | 6.573 | 21.764 | 1.00 | 16.36 |
| ATOM | 677 | CA | PHE | A | 619 | −2.040 | 6.942 | 22.316 | 1.00 | 18.60 |
| ATOM | 678 | CB | PHE | A | 619 | −2.205 | 8.224 | 23.142 | 1.00 | 21.01 |
| ATOM | 679 | CG | PHE | A | 619 | −0.989 | 8.594 | 23.938 | 1.00 | 22.31 |
| ATOM | 680 | CD1 | PHE | A | 619 | 0.166 | 9.041 | 23.305 | 1.00 | 21.99 |
| ATOM | 681 | CD2 | PHE | A | 619 | −0.995 | 8.475 | 25.326 | 1.00 | 22.04 |
| ATOM | 682 | CE1 | PHE | A | 619 | 1.304 | 9.366 | 24.045 | 1.00 | 21.42 |
| ATOM | 683 | CE2 | PHE | A | 619 | 0.139 | 8.798 | 26.076 | 1.00 | 21.94 |
| ATOM | 684 | CZ | PHE | A | 619 | 1.292 | 9.246 | 25.426 | 1.00 | 21.32 |
| ATOM | 685 | C | PHE | A | 619 | −1.459 | 5.816 | 23.176 | 1.00 | 17.86 |
| ATOM | 686 | O | PHE | A | 619 | −0.285 | 5.470 | 23.040 | 1.00 | 17.74 |
| ATOM | 687 | N | ASN | A | 620 | −2.281 | 5.239 | 24.049 | 1.00 | 17.68 |
| ATOM | 688 | CA | ASN | A | 620 | −1.814 | 4.160 | 24.920 | 1.00 | 18.84 |
| ATOM | 689 | CB | ASN | A | 620 | −2.868 | 3.793 | 25.972 | 1.00 | 22.20 |
| ATOM | 690 | CG | ASN | A | 620 | −3.028 | 4.867 | 27.037 | 1.00 | 22.41 |
| ATOM | 691 | OD1 | ASN | A | 620 | −2.078 | 5.578 | 27.354 | 1.00 | 22.93 |
| ATOM | 692 | ND2 | ASN | A | 620 | −4.220 | 4.969 | 27.611 | 1.00 | 21.57 |
| ATOM | 693 | C | ASN | A | 620 | −1.450 | 2.921 | 24.113 | 1.00 | 18.91 |
| ATOM | 694 | O | ASN | A | 620 | −0.514 | 2.201 | 24.461 | 1.00 | 18.42 |
| ATOM | 695 | N | THR | A | 621 | −2.184 | 2.682 | 23.031 | 1.00 | 16.41 |
| ATOM | 696 | CA | THR | A | 621 | −1.901 | 1.519 | 22.191 | 1.00 | 16.82 |
| ATOM | 697 | CB | THR | A | 621 | −2.965 | 1.360 | 21.093 | 1.00 | 17.95 |
| ATOM | 698 | OG1 | THR | A | 621 | −4.246 | 1.190 | 21.704 | 1.00 | 17.42 |
| ATOM | 699 | CG2 | THR | A | 621 | −2.664 | 0.130 | 20.233 | 1.00 | 17.02 |
| ATOM | 700 | C | THR | A | 621 | −0.526 | 1.693 | 21.556 | 1.00 | 18.34 |
| ATOM | 701 | O | THR | A | 621 | 0.276 | 0.749 | 21.505 | 1.00 | 18.19 |
| ATOM | 702 | N | ALA | A | 622 | −0.249 | 2.901 | 21.085 | 1.00 | 15.98 |
| ATOM | 703 | CA | ALA | A | 622 | 1.041 | 3.211 | 20.479 | 1.00 | 18.35 |
| ATOM | 704 | CB | ALA | A | 622 | 1.005 | 4.592 | 19.864 | 1.00 | 16.90 |
| ATOM | 705 | C | ALA | A | 622 | 2.151 | 3.138 | 21.529 | 1.00 | 17.61 |
| ATOM | 706 | O | ALA | A | 622 | 3.242 | 2.656 | 21.248 | 1.00 | 16.57 |
| ATOM | 707 | N | GLN | A | 623 | 1.876 | 3.622 | 22.740 | 1.00 | 17.78 |
| ATOM | 708 | CA | GLN | A | 623 | 2.878 | 3.578 | 23.803 | 1.00 | 19.17 |
| ATOM | 709 | CB | GLN | A | 623 | 2.351 | 4.260 | 25.071 | 1.00 | 19.99 |
| ATOM | 710 | CG | GLN | A | 623 | 3.361 | 4.335 | 26.219 | 1.00 | 19.42 |
| ATOM | 711 | CD | GLN | A | 623 | 3.358 | 3.103 | 27.110 | 1.00 | 20.35 |
| ATOM | 712 | OE1 | GLN | A | 623 | 2.302 | 2.595 | 27.488 | 1.00 | 19.39 |
| ATOM | 713 | NE2 | GLN | A | 623 | 4.547 | 2.634 | 27.475 | 1.00 | 19.48 |
| ATOM | 714 | C | GLN | A | 623 | 3.251 | 2.137 | 24.114 | 1.00 | 19.45 |
| ATOM | 715 | O | GLN | A | 623 | 4.420 | 1.824 | 24.358 | 1.00 | 19.89 |
| ATOM | 716 | N | CYS | A | 624 | 2.258 | 1.256 | 24.118 | 1.00 | 17.26 |
| ATOM | 717 | CA | CYS | A | 624 | 2.533 | −0.146 | 24.385 | 1.00 | 19.37 |
| ATOM | 718 | CB | CYS | A | 624 | 1.234 | −0.938 | 24.530 | 1.00 | 19.88 |
| ATOM | 719 | SG | CYS | A | 624 | 1.499 | −2.658 | 25.026 | 1.00 | 19.62 |
| ATOM | 720 | C | CYS | A | 624 | 3.369 | −0.708 | 23.237 | 1.00 | 18.34 |
| ATOM | 721 | O | CYS | A | 624 | 4.255 | −1.528 | 23.460 | 1.00 | 18.71 |
| ATOM | 722 | N | MET | A | 625 | 3.094 | −0.258 | 22.015 | 1.00 | 19.30 |
| ATOM | 723 | CA | MET | A | 625 | 3.844 | −0.720 | 20.843 | 1.00 | 18.88 |
| ATOM | 724 | CB | MET | A | 625 | 3.259 | −0.102 | 19.567 | 1.00 | 21.69 |
| ATOM | 725 | CG | MET | A | 625 | 3.952 | −0.521 | 18.267 | 1.00 | 19.33 |
| ATOM | 726 | SD | MET | A | 625 | 3.854 | −2.291 | 17.926 | 1.00 | 18.96 |
| ATOM | 727 | CE | MET | A | 625 | 2.148 | −2.414 | 17.292 | 1.00 | 20.28 |
| ATOM | 728 | C | MET | A | 625 | 5.307 | −0.296 | 21.019 | 1.00 | 20.73 |
| ATOM | 729 | O | MET | A | 625 | 6.226 | −1.097 | 20.832 | 1.00 | 19.61 |
| ATOM | 730 | N | PHE | A | 626 | 5.515 | 0.968 | 21.381 | 1.00 | 21.46 |
| ATOM | 731 | CA | PHE | A | 626 | 6.862 | 1.487 | 21.604 | 1.00 | 22.24 |
| ATOM | 732 | CB | PHE | A | 626 | 6.810 | 2.973 | 22.003 | 1.00 | 18.30 |
| ATOM | 733 | CG | PHE | A | 626 | 8.162 | 3.566 | 22.325 | 1.00 | 18.77 |
| ATOM | 734 | CD1 | PHE | A | 626 | 8.662 | 3.539 | 23.628 | 1.00 | 19.77 |
| ATOM | 735 | CD2 | PHE | A | 626 | 8.943 | 4.126 | 21.321 | 1.00 | 19.03 |
| ATOM | 736 | CE1 | PHE | A | 626 | 9.920 | 4.060 | 23.921 | 1.00 | 19.58 |
| ATOM | 737 | CE2 | PHE | A | 626 | 10.207 | 4.650 | 21.607 | 1.00 | 19.50 |
| ATOM | 738 | CZ | PHE | A | 626 | 10.693 | 4.616 | 22.907 | 1.00 | 17.99 |
| ATOM | 739 | C | PHE | A | 626 | 7.578 | 0.684 | 22.685 | 1.00 | 22.81 |
| ATOM | 740 | O | PHE | A | 626 | 8.747 | 0.321 | 22.528 | 1.00 | 22.67 |
| ATOM | 741 | N | ALA | A | 627 | 6.878 | 0.405 | 23.781 | 1.00 | 23.57 |
| ATOM | 742 | CA | ALA | A | 627 | 7.460 | −0.351 | 24.883 | 1.00 | 24.17 |
| ATOM | 743 | CB | ALA | A | 627 | 6.511 | −0.357 | 26.082 | 1.00 | 26.38 |
| ATOM | 744 | C | ALA | A | 627 | 7.794 | −1.787 | 24.477 | 1.00 | 24.26 |
| ATOM | 745 | O | ALA | A | 627 | 8.854 | −2.305 | 24.834 | 1.00 | 25.36 |
| ATOM | 746 | N | ALA | A | 628 | 6.898 | −2.428 | 23.728 | 1.00 | 21.99 |
| ATOM | 747 | CA | ALA | A | 628 | 7.127 | −3.806 | 23.296 | 1.00 | 22.22 |
| ATOM | 748 | CB | ALA | A | 628 | 5.884 | −4.364 | 22.609 | 1.00 | 18.57 |
| ATOM | 749 | C | ALA | A | 628 | 8.315 | −3.879 | 22.350 | 1.00 | 23.09 |
| ATOM | 750 | O | ALA | A | 628 | 9.085 | −4.838 | 22.382 | 1.00 | 22.87 |
| ATOM | 751 | N | LEU | A | 629 | 8.462 | −2.864 | 21.506 | 1.00 | 21.79 |
| ATOM | 752 | CA | LEU | A | 629 | 9.565 | −2.835 | 20.555 | 1.00 | 24.56 |
| ATOM | 753 | CB | LEU | A | 629 | 9.338 | −1.737 | 19.521 | 1.00 | 24.39 |
| ATOM | 754 | CG | LEU | A | 629 | 8.155 | −1.980 | 18.582 | 1.00 | 25.70 |
| ATOM | 755 | CD1 | LEU | A | 629 | 7.906 | −0.753 | 17.734 | 1.00 | 25.30 |
| ATOM | 756 | CD2 | LEU | A | 629 | 8.446 | −3.200 | 17.711 | 1.00 | 26.08 |
| ATOM | 757 | C | LEU | A | 629 | 10.897 | −2.608 | 21.256 | 1.00 | 25.12 |
| ATOM | 758 | O | LEU | A | 629 | 11.898 | −3.243 | 20.920 | 1.00 | 25.35 |
| ATOM | 759 | N | LYS | A | 630 | 10.907 | −1.705 | 22.234 | 1.00 | 32.90 |
| ATOM | 760 | CA | LYS | A | 630 | 12.126 | −1.394 | 22.981 | 1.00 | 33.87 |
| ATOM | 761 | CB | LYS | A | 630 | 12.033 | 0.012 | 23.573 | 1.00 | 31.73 |
| ATOM | 762 | CG | LYS | A | 630 | 12.137 | 1.116 | 22.544 | 1.00 | 31.51 |
| ATOM | 763 | CD | LYS | A | 630 | 13.494 | 1.089 | 21.868 | 1.00 | 32.17 |
| ATOM | 764 | CE | LYS | A | 630 | 13.682 | 2.292 | 20.966 | 1.00 | 31.52 |
| ATOM | 765 | NZ | LYS | A | 630 | 14.984 | 2.243 | 20.254 | 1.00 | 31.88 |
| ATOM | 766 | C | LYS | A | 630 | 12.416 | −2.394 | 24.096 | 1.00 | 34.55 |
| ATOM | 767 | O | LYS | A | 630 | 13.176 | −3.347 | 23.906 | 1.00 | 35.07 |
| ATOM | 768 | N | ALA | A | 631 | 11.814 | −2.168 | 25.260 | 1.00 | 30.54 |
| ATOM | 769 | CA | ALA | A | 631 | 12.005 | −3.047 | 26.406 | 1.00 | 30.93 |
| ATOM | 770 | CB | ALA | A | 631 | 11.175 | −2.548 | 27.590 | 1.00 | 31.25 |
| ATOM | 771 | C | ALA | A | 631 | 11.630 | −4.492 | 26.070 | 1.00 | 30.76 |
| ATOM | 772 | O | ALA | A | 631 | 12.270 | −5.432 | 26.538 | 1.00 | 30.38 |
| ATOM | 773 | N | GLY | A | 632 | 10.588 | −4.660 | 25.257 | 1.00 | 30.21 |
| ATOM | 774 | CA | GLY | A | 632 | 10.147 | −5.991 | 24.871 | 1.00 | 30.31 |
| ATOM | 775 | C | GLY | A | 632 | 11.073 | −6.644 | 23.860 | 1.00 | 30.73 |
| ATOM | 776 | O | GLY | A | 632 | 10.975 | −7.843 | 23.592 | 1.00 | 30.32 |
| ATOM | 777 | N | LYS | A | 633 | 11.967 | −5.848 | 23.287 | 1.00 | 31.93 |
| ATOM | 778 | CA | LYS | A | 633 | 12.937 | −6.346 | 22.315 | 1.00 | 32.77 |
| ATOM | 779 | CB | LYS | A | 633 | 13.858 | −7.374 | 22.979 | 1.00 | 44.15 |
| ATOM | 780 | CG | LYS | A | 633 | 14.875 | −6.775 | 23.929 | 1.00 | 44.47 |
| ATOM | 781 | CD | LYS | A | 633 | 15.841 | −7.835 | 24.426 | 1.00 | 44.54 |
| ATOM | 782 | CE | LYS | A | 633 | 17.052 | −7.197 | 25.079 | 1.00 | 44.74 |
| ATOM | 783 | NZ | LYS | A | 633 | 17.770 | −6.316 | 24.114 | 1.00 | 44.90 |
| ATOM | 784 | C | LYS | A | 633 | 12.352 | −6.948 | 21.041 | 1.00 | 32.00 |
| ATOM | 785 | O | LYS | A | 633 | 12.862 | −7.944 | 20.522 | 1.00 | 32.88 |
| ATOM | 786 | N | ILE | A | 634 | 11.292 | −6.338 | 20.527 | 1.00 | 26.60 |
| ATOM | 787 | CA | ILE | A | 634 | 10.670 | −6.818 | 19.305 | 1.00 | 25.39 |
| ATOM | 788 | CB | ILE | A | 634 | 9.124 | −6.695 | 19.400 | 1.00 | 26.41 |
| ATOM | 789 | CG2 | ILE | A | 634 | 8.473 | −7.074 | 18.076 | 1.00 | 27.54 |
| ATOM | 790 | CG1 | ILE | A | 634 | 8.607 | −7.612 | 20.510 | 1.00 | 27.29 |
| ATOM | 791 | CD1 | ILE | A | 634 | 7.113 | −7.543 | 20.725 | 1.00 | 27.31 |
| ATOM | 792 | C | ILE | A | 634 | 11.200 | −6.013 | 18.117 | 1.00 | 25.00 |
| ATOM | 793 | O | ILE | A | 634 | 11.127 | −6.446 | 16.968 | 1.00 | 25.10 |
| ATOM | 794 | N | GLN | A | 635 | 11.770 | −4.850 | 18.409 | 1.00 | 23.96 |
| ATOM | 795 | CA | GLN | A | 635 | 12.305 | −3.975 | 17.377 | 1.00 | 23.37 |
| ATOM | 796 | CB | GLN | A | 635 | 12.904 | −2.716 | 18.010 | 1.00 | 27.98 |
| ATOM | 797 | CG | GLN | A | 635 | 13.476 | −1.740 | 17.002 | 1.00 | 28.57 |
| ATOM | 798 | CD | GLN | A | 635 | 14.111 | −0.516 | 17.645 | 1.00 | 28.17 |
| ATOM | 799 | OE1 | GLN | A | 635 | 14.635 | 0.354 | 16.953 | 1.00 | 29.49 |
| ATOM | 800 | NE2 | GLN | A | 635 | 14.059 | −0.444 | 18.967 | 1.00 | 27.30 |
| ATOM | 801 | C | GLN | A | 635 | 13.368 | −4.672 | 16.535 | 1.00 | 24.49 |
| ATOM | 802 | O | GLN | A | 635 | 13.445 | −4.468 | 15.324 | 1.00 | 24.24 |
| ATOM | 803 | N | ASN | A | 636 | 14.180 | −5.493 | 17.195 | 1.00 | 30.08 |
| ATOM | 804 | CA | ASN | A | 636 | 15.266 | −6.222 | 16.547 | 1.00 | 31.17 |
| ATOM | 805 | CB | ASN | A | 636 | 16.172 | −6.829 | 17.618 | 1.00 | 31.58 |
| ATOM | 806 | CG | ASN | A | 636 | 16.612 | −5.811 | 18.651 | 1.00 | 32.71 |
| ATOM | 807 | OD1 | ASN | A | 636 | 17.739 | −5.309 | 18.607 | 1.00 | 32.53 |
| ATOM | 808 | ND2 | ASN | A | 636 | 15.716 | −5.490 | 19.585 | 1.00 | 31.94 |
| ATOM | 809 | C | ASN | A | 636 | 14.782 | −7.330 | 15.617 | 1.00 | 31.51 |
| ATOM | 810 | O | ASN | A | 636 | 15.553 | −7.853 | 14.809 | 1.00 | 31.97 |
| ATOM | 811 | N | LYS | A | 637 | 13.510 | −7.690 | 15.732 | 1.00 | 28.95 |
| ATOM | 812 | CA | LYS | A | 637 | 12.944 | −8.750 | 14.904 | 1.00 | 28.44 |
| ATOM | 813 | CB | LYS | A | 637 | 11.983 | −9.596 | 15.740 | 1.00 | 31.06 |
| ATOM | 814 | CG | LYS | A | 637 | 12.590 | −10.196 | 16.993 | 1.00 | 30.31 |
| ATOM | 815 | CD | LYS | A | 637 | 11.502 | −10.770 | 17.889 | 1.00 | 30.88 |
| ATOM | 816 | CE | LYS | A | 637 | 12.072 | −11.316 | 19.186 | 1.00 | 31.13 |
| ATOM | 817 | NZ | LYS | A | 637 | 12.956 | −12.494 | 18.960 | 1.00 | 31.42 |
| ATOM | 818 | C | LYS | A | 637 | 12.200 | −8.219 | 13.683 | 1.00 | 28.47 |
| ATOM | 819 | O | LYS | A | 637 | 11.696 | −9.003 | 12.875 | 1.00 | 29.67 |
| ATOM | 820 | N | LEU | A | 638 | 12.137 | −6.898 | 13.539 | 1.00 | 27.41 |
| ATOM | 821 | CA | LEU | A | 638 | 11.401 | −6.297 | 12.427 | 1.00 | 27.76 |
| ATOM | 822 | CB | LEU | A | 638 | 10.187 | −5.546 | 12.977 | 1.00 | 24.26 |
| ATOM | 823 | CG | LEU | A | 638 | 9.349 | −6.293 | 14.016 | 1.00 | 24.56 |
| ATOM | 824 | CD1 | LEU | A | 638 | 8.265 | −5.366 | 14.549 | 1.00 | 25.02 |
| ATOM | 825 | CD2 | LEU | A | 638 | 8.740 | −7.549 | 13.396 | 1.00 | 24.12 |
| ATOM | 826 | C | LEU | A | 638 | 12.203 | −5.348 | 11.543 | 1.00 | 26.64 |
| ATOM | 827 | O | LEU | A | 638 | 13.301 | −4.925 | 11.896 | 1.00 | 28.43 |
| ATOM | 828 | N | THR | A | 639 | 11.630 | −5.011 | 10.389 | 1.00 | 23.44 |
| ATOM | 829 | CA | THR | A | 639 | 12.253 | −4.093 | 9.441 | 1.00 | 22.41 |
| ATOM | 830 | CB | THR | A | 639 | 11.772 | −4.343 | 8.004 | 1.00 | 28.95 |
| ATOM | 831 | OG1 | THR | A | 639 | 10.378 | −4.021 | 7.908 | 1.00 | 28.33 |
| ATOM | 832 | CG2 | THR | A | 639 | 11.982 | −5.794 | 7.611 | 1.00 | 28.40 |
| ATOM | 833 | C | THR | A | 639 | 11.870 | −2.662 | 9.798 | 1.00 | 22.44 |
| ATOM | 834 | O | THR | A | 639 | 10.921 | −2.445 | 10.551 | 1.00 | 23.44 |
| ATOM | 835 | N | ASP | A | 640 | 12.601 | −1.695 | 9.250 | 1.00 | 24.48 |
| ATOM | 836 | CA | ASP | A | 640 | 12.321 | −0.288 | 9.508 | 1.00 | 24.67 |
| ATOM | 837 | CB | ASP | A | 640 | 13.313 | 0.604 | 8.747 | 1.00 | 30.57 |
| ATOM | 838 | CG | ASP | A | 640 | 14.693 | 0.642 | 9.393 | 1.00 | 30.89 |
| ATOM | 839 | OD1 | ASP | A | 640 | 15.634 | 1.146 | 8.743 | 1.00 | 30.85 |
| ATOM | 840 | OD2 | ASP | A | 640 | 14.838 | 0.184 | 10.548 | 1.00 | 30.86 |
| ATOM | 841 | C | ASP | A | 640 | 10.895 | 0.038 | 9.074 | 1.00 | 24.64 |
| ATOM | 842 | O | ASP | A | 640 | 10.177 | 0.761 | 9.765 | 1.00 | 24.23 |
| ATOM | 843 | N | LEU | A | 641 | 10.491 | −0.513 | 7.931 | 1.00 | 24.25 |
| ATOM | 844 | CA | LEU | A | 641 | 9.153 | −0.291 | 7.392 | 1.00 | 23.81 |
| ATOM | 845 | CB | LEU | A | 641 | 9.066 | −0.863 | 5.975 | 1.00 | 25.82 |
| ATOM | 846 | CG | LEU | A | 641 | 9.326 | 0.063 | 4.781 | 1.00 | 27.50 |
| ATOM | 847 | CD1 | LEU | A | 641 | 10.267 | 1.188 | 5.137 | 1.00 | 25.96 |
| ATOM | 848 | CD2 | LEU | A | 641 | 9.858 | −0.767 | 3.629 | 1.00 | 26.21 |
| ATOM | 849 | C | LEU | A | 641 | 8.074 | −0.914 | 8.269 | 1.00 | 23.08 |
| ATOM | 850 | O | LEU | A | 641 | 7.013 | −0.332 | 8.464 | 1.00 | 24.13 |
| ATOM | 851 | N | GLU | A | 642 | 8.336 | −2.110 | 8.778 | 1.00 | 23.58 |
| ATOM | 852 | CA | GLU | A | 642 | 7.371 | −2.771 | 9.639 | 1.00 | 24.03 |
| ATOM | 853 | CB | GLU | A | 642 | 7.847 | −4.188 | 9.966 | 1.00 | 29.79 |
| ATOM | 854 | CG | GLU | A | 642 | 7.607 | −5.150 | 8.816 | 1.00 | 30.52 |
| ATOM | 855 | CD | GLU | A | 642 | 8.276 | −6.492 | 9.004 | 1.00 | 30.88 |
| ATOM | 856 | OE1 | GLU | A | 642 | 7.928 | −7.424 | 8.246 | 1.00 | 30.84 |
| ATOM | 857 | OE2 | GLU | A | 642 | 9.147 | −6.615 | 9.893 | 1.00 | 30.40 |
| ATOM | 858 | C | GLU | A | 642 | 7.166 | −1.947 | 10.911 | 1.00 | 24.17 |
| ATOM | 859 | O | GLU | A | 642 | 6.043 | −1.816 | 11.404 | 1.00 | 24.58 |
| ATOM | 860 | N | ILE | A | 643 | 8.260 | −1.382 | 11.419 | 1.00 | 21.47 |
| ATOM | 861 | CA | ILE | A | 643 | 8.230 | −0.555 | 12.625 | 1.00 | 21.07 |
| ATOM | 862 | CB | ILE | A | 643 | 9.659 | −0.184 | 13.062 | 1.00 | 24.97 |
| ATOM | 863 | CG2 | ILE | A | 643 | 9.614 | 0.833 | 14.195 | 1.00 | 23.30 |
| ATOM | 864 | CG1 | ILE | A | 643 | 10.409 | −1.444 | 13.502 | 1.00 | 23.69 |
| ATOM | 865 | CD1 | ILE | A | 643 | 11.900 | −1.229 | 13.673 | 1.00 | 24.32 |
| ATOM | 866 | C | ILE | A | 643 | 7.440 | 0.727 | 12.350 | 1.00 | 20.73 |
| ATOM | 867 | O | ILE | A | 643 | 6.536 | 1.088 | 13.109 | 1.00 | 20.07 |
| ATOM | 868 | N | LEU | A | 644 | 7.792 | 1.405 | 11.259 | 1.00 | 21.45 |
| ATOM | 869 | CA | LEU | A | 644 | 7.122 | 2.632 | 10.843 | 1.00 | 22.42 |
| ATOM | 870 | CB | LEU | A | 644 | 7.707 | 3.112 | 9.510 | 1.00 | 22.90 |
| ATOM | 871 | CG | LEU | A | 644 | 7.028 | 4.294 | 8.816 | 1.00 | 23.31 |
| ATOM | 872 | CD1 | LEU | A | 644 | 7.162 | 5.546 | 9.670 | 1.00 | 23.35 |
| ATOM | 873 | CD2 | LEU | A | 644 | 7.671 | 4.506 | 7.449 | 1.00 | 22.87 |
| ATOM | 874 | C | LEU | A | 644 | 5.623 | 2.400 | 10.686 | 1.00 | 22.48 |
| ATOM | 875 | O | LEU | A | 644 | 4.809 | 3.178 | 11.183 | 1.00 | 21.76 |
| ATOM | 876 | N | ALA | A | 645 | 5.262 | 1.327 | 9.993 | 1.00 | 19.64 |
| ATOM | 877 | CA | ALA | A | 645 | 3.858 | 1.003 | 9.770 | 1.00 | 19.40 |
| ATOM | 878 | CB | ALA | A | 645 | 3.747 | −0.144 | 8.759 | 1.00 | 19.42 |
| ATOM | 879 | C | ALA | A | 645 | 3.110 | 0.643 | 11.062 | 1.00 | 18.96 |
| ATOM | 880 | O | ALA | A | 645 | 1.946 | 1.016 | 11.238 | 1.00 | 16.29 |
| ATOM | 881 | N | LEU | A | 646 | 3.767 | −0.073 | 11.970 | 1.00 | 15.67 |
| ATOM | 882 | CA | LEU | A | 646 | 3.113 | −0.458 | 13.218 | 1.00 | 17.59 |
| ATOM | 883 | CB | LEU | A | 646 | 3.972 | −1.464 | 13.982 | 1.00 | 21.09 |
| ATOM | 884 | CG | LEU | A | 646 | 4.011 | −2.880 | 13.401 | 1.00 | 20.20 |
| ATOM | 885 | CD1 | LEU | A | 646 | 5.105 | −3.678 | 14.102 | 1.00 | 20.69 |
| ATOM | 886 | CD2 | LEU | A | 646 | 2.654 | −3.552 | 13.561 | 1.00 | 21.23 |
| ATOM | 887 | C | LEU | A | 646 | 2.826 | 0.750 | 14.112 | 1.00 | 16.19 |
| ATOM | 888 | O | LEU | A | 646 | 1.770 | 0.824 | 14.748 | 1.00 | 17.93 |
| ATOM | 889 | N | LEU | A | 647 | 3.767 | 1.687 | 14.168 | 1.00 | 20.47 |
| ATOM | 890 | CA | LEU | A | 647 | 3.578 | 2.875 | 15.000 | 1.00 | 18.59 |
| ATOM | 891 | CB | LEU | A | 647 | 4.874 | 3.682 | 15.097 | 1.00 | 19.99 |
| ATOM | 892 | CG | LEU | A | 647 | 4.819 | 4.890 | 16.042 | 1.00 | 19.72 |
| ATOM | 893 | CD1 | LEU | A | 647 | 4.422 | 4.432 | 17.445 | 1.00 | 21.01 |
| ATOM | 894 | CD2 | LEU | A | 647 | 6.168 | 5.563 | 16.078 | 1.00 | 21.38 |
| ATOM | 895 | C | LEU | A | 647 | 2.447 | 3.735 | 14.436 | 1.00 | 19.57 |
| ATOM | 896 | O | LEU | A | 647 | 1.583 | 4.199 | 15.183 | 1.00 | 19.14 |
| ATOM | 897 | N | ILE | A | 648 | 2.447 | 3.935 | 13.120 | 1.00 | 17.04 |
| ATOM | 898 | CA | ILE | A | 648 | 1.393 | 4.714 | 12.475 | 1.00 | 16.34 |
| ATOM | 899 | CB | ILE | A | 648 | 1.671 | 4.884 | 10.962 | 1.00 | 16.27 |
| ATOM | 900 | CG2 | ILE | A | 648 | 0.428 | 5.441 | 10.251 | 1.00 | 17.73 |
| ATOM | 901 | CG1 | ILE | A | 648 | 2.895 | 5.787 | 10.770 | 1.00 | 18.41 |
| ATOM | 902 | CD1 | ILE | A | 648 | 3.350 | 5.921 | 9.339 | 1.00 | 17.00 |
| ATOM | 903 | C | ILE | A | 648 | 0.060 | 3.999 | 12.674 | 1.00 | 17.17 |
| ATOM | 904 | O | ILE | A | 648 | −0.946 | 4.620 | 13.027 | 1.00 | 16.88 |
| ATOM | 905 | N | ALA | A | 649 | 0.056 | 2.684 | 12.477 | 1.00 | 17.97 |
| ATOM | 906 | CA | ALA | A | 649 | −1.171 | 1.912 | 12.634 | 1.00 | 18.14 |
| ATOM | 907 | CB | ALA | A | 649 | −0.927 | 0.460 | 12.228 | 1.00 | 19.75 |
| ATOM | 908 | C | ALA | A | 649 | −1.724 | 1.977 | 14.060 | 1.00 | 17.69 |
| ATOM | 909 | O | ALA | A | 649 | −2.928 | 2.180 | 14.266 | 1.00 | 17.10 |
| ATOM | 910 | N | ALA | A | 650 | −0.850 | 1.802 | 15.047 | 1.00 | 16.14 |
| ATOM | 911 | CA | ALA | A | 650 | −1.276 | 1.846 | 16.438 | 1.00 | 15.98 |
| ATOM | 912 | CB | ALA | A | 650 | −0.070 | 1.703 | 17.365 | 1.00 | 18.40 |
| ATOM | 913 | C | ALA | A | 650 | −1.987 | 3.175 | 16.708 | 1.00 | 16.55 |
| ATOM | 914 | O | ALA | A | 650 | −3.049 | 3.214 | 17.327 | 1.00 | 17.85 |
| ATOM | 915 | N | LEU | A | 651 | −1.401 | 4.259 | 16.226 | 1.00 | 17.09 |
| ATOM | 916 | CA | LEU | A | 651 | −1.974 | 5.589 | 16.438 | 1.00 | 17.07 |
| ATOM | 917 | CB | LEU | A | 651 | −0.956 | 6.659 | 16.056 | 1.00 | 20.77 |
| ATOM | 918 | CG | LEU | A | 651 | 0.304 | 6.766 | 16.906 | 1.00 | 20.44 |
| ATOM | 919 | CD1 | LEU | A | 651 | 1.326 | 7.596 | 16.156 | 1.00 | 20.57 |
| ATOM | 920 | CD2 | LEU | A | 651 | −0.036 | 7.402 | 18.255 | 1.00 | 20.14 |
| ATOM | 921 | C | LEU | A | 651 | −3.246 | 5.846 | 15.648 | 1.00 | 17.89 |
| ATOM | 922 | O | LEU | A | 651 | −4.133 | 6.569 | 16.102 | 1.00 | 17.40 |
| ATOM | 923 | N | SER | A | 652 | −3.335 | 5.248 | 14.465 | 1.00 | 14.66 |
| ATOM | 924 | CA | SER | A | 652 | −4.484 | 5.470 | 13.591 | 1.00 | 16.84 |
| ATOM | 925 | CB | SER | A | 652 | −4.013 | 5.620 | 12.141 | 1.00 | 19.10 |
| ATOM | 926 | OG | SER | A | 652 | −2.948 | 6.535 | 12.014 | 1.00 | 18.78 |
| ATOM | 927 | C | SER | A | 652 | −5.549 | 4.386 | 13.596 | 1.00 | 17.02 |
| ATOM | 928 | O | SER | A | 652 | −6.593 | 4.574 | 12.987 | 1.00 | 16.48 |
| ATOM | 929 | N | HIS | A | 653 | −5.302 | 3.280 | 14.292 | 1.00 | 19.51 |
| ATOM | 930 | CA | HIS | A | 653 | −6.219 | 2.143 | 14.241 | 1.00 | 18.51 |
| ATOM | 931 | CB | HIS | A | 653 | −5.607 | 0.939 | 14.972 | 1.00 | 15.82 |
| ATOM | 932 | CG | HIS | A | 653 | −5.944 | 0.882 | 16.427 | 1.00 | 16.43 |
| ATOM | 933 | CD2 | HIS | A | 653 | −6.947 | 0.252 | 17.079 | 1.00 | 15.65 |
| ATOM | 934 | ND1 | HIS | A | 653 | −5.237 | 1.574 | 17.385 | 1.00 | 14.87 |
| ATOM | 935 | CE1 | HIS | A | 653 | −5.794 | 1.376 | 18.567 | 1.00 | 15.10 |
| ATOM | 936 | NE2 | HIS | A | 653 | −6.838 | 0.580 | 18.406 | 1.00 | 15.73 |
| ATOM | 937 | C | HIS | A | 653 | −7.681 | 2.284 | 14.652 | 1.00 | 19.28 |
| ATOM | 938 | O | HIS | A | 653 | −8.486 | 1.441 | 14.289 | 1.00 | 17.55 |
| ATOM | 939 | N | ASP | A | 654 | −8.039 | 3.329 | 15.395 | 1.00 | 14.03 |
| ATOM | 940 | CA | ASP | A | 654 | −9.434 | 3.505 | 15.811 | 1.00 | 13.68 |
| ATOM | 941 | CB | ASP | A | 654 | −9.576 | 3.404 | 17.342 | 1.00 | 17.23 |
| ATOM | 942 | CG | ASP | A | 654 | −9.893 | 1.995 | 17.816 | 1.00 | 16.81 |
| ATOM | 943 | OD1 | ASP | A | 654 | −10.559 | 1.251 | 17.063 | 1.00 | 16.45 |
| ATOM | 944 | OD2 | ASP | A | 654 | −9.510 | 1.647 | 18.953 | 1.00 | 16.54 |
| ATOM | 945 | C | ASP | A | 654 | −9.986 | 4.858 | 15.351 | 1.00 | 14.35 |
| ATOM | 946 | O | ASP | A | 654 | −11.004 | 5.325 | 15.870 | 1.00 | 15.00 |
| ATOM | 947 | N | LEU | A | 655 | −9.342 | 5.461 | 14.353 | 1.00 | 14.26 |
| ATOM | 948 | CA | LEU | A | 655 | −9.745 | 6.777 | 13.838 | 1.00 | 15.05 |
| ATOM | 949 | CB | LEU | A | 655 | −8.976 | 7.071 | 12.542 | 1.00 | 21.81 |
| ATOM | 950 | CG | LEU | A | 655 | −8.028 | 8.263 | 12.357 | 1.00 | 25.53 |
| ATOM | 951 | CD1 | LEU | A | 655 | −7.712 | 8.958 | 13.671 | 1.00 | 22.91 |
| ATOM | 952 | CD2 | LEU | A | 655 | −6.766 | 7.763 | 11.665 | 1.00 | 22.84 |
| ATOM | 953 | C | LEU | A | 655 | −11.257 | 6.941 | 13.591 | 1.00 | 16.32 |
| ATOM | 954 | O | LEU | A | 655 | −11.889 | 6.120 | 12.923 | 1.00 | 15.44 |
| ATOM | 955 | N | ASP | A | 656 | −11.826 | 8.013 | 14.147 | 1.00 | 16.42 |
| ATOM | 956 | CA | ASP | A | 656 | −13.251 | 8.326 | 13.988 | 1.00 | 17.71 |
| ATOM | 957 | CB | ASP | A | 656 | −13.556 | 8.647 | 12.508 | 1.00 | 20.19 |
| ATOM | 958 | CG | ASP | A | 656 | −14.905 | 9.353 | 12.312 | 1.00 | 20.24 |
| ATOM | 959 | OD1 | ASP | A | 656 | −15.243 | 10.252 | 13.110 | 1.00 | 19.07 |
| ATOM | 960 | OD2 | ASP | A | 656 | −15.623 | 9.021 | 11.344 | 1.00 | 19.47 |
| ATOM | 961 | C | ASP | A | 656 | −14.197 | 7.232 | 14.496 | 1.00 | 16.50 |
| ATOM | 962 | O | ASP | A | 656 | −15.295 | 7.060 | 13.971 | 1.00 | 16.42 |
| ATOM | 963 | N | HIS | A | 657 | −13.777 | 6.502 | 15.523 | 1.00 | 16.01 |
| ATOM | 964 | CA | HIS | A | 657 | −14.627 | 5.454 | 16.073 | 1.00 | 15.64 |
| ATOM | 965 | CB | HIS | A | 657 | −13.902 | 4.710 | 17.196 | 1.00 | 18.44 |
| ATOM | 966 | CG | HIS | A | 657 | −14.585 | 3.446 | 17.606 | 1.00 | 19.68 |
| ATOM | 967 | CD2 | HIS | A | 657 | −15.832 | 3.224 | 18.080 | 1.00 | 15.96 |
| ATOM | 968 | ND1 | HIS | A | 657 | −13.979 | 2.209 | 17.524 | 1.00 | 19.05 |
| ATOM | 969 | CE1 | HIS | A | 657 | −14.825 | 1.281 | 17.930 | 1.00 | 16.34 |
| ATOM | 970 | NE2 | HIS | A | 657 | −15.958 | 1.871 | 18.271 | 1.00 | 21.05 |
| ATOM | 971 | C | HIS | A | 657 | −15.893 | 6.126 | 16.611 | 1.00 | 17.49 |
| ATOM | 972 | O | HIS | A | 657 | −15.826 | 7.093 | 17.377 | 1.00 | 16.58 |
| ATOM | 973 | N | PRO | A | 658 | −17.068 | 5.625 | 16.216 | 1.00 | 19.29 |
| ATOM | 974 | CD | PRO | A | 658 | −17.281 | 4.579 | 15.199 | 1.00 | 17.12 |
| ATOM | 975 | CA | PRO | A | 658 | −18.346 | 6.189 | 16.659 | 1.00 | 19.86 |
| ATOM | 976 | CB | PRO | A | 658 | −19.313 | 5.701 | 15.595 | 1.00 | 16.51 |
| ATOM | 977 | CG | PRO | A | 658 | −18.772 | 4.311 | 15.313 | 1.00 | 15.25 |
| ATOM | 978 | C | PRO | A | 658 | −18.789 | 5.780 | 18.058 | 1.00 | 19.74 |
| ATOM | 979 | O | PRO | A | 658 | −19.745 | 6.335 | 18.589 | 1.00 | 20.03 |
| ATOM | 980 | N | GLY | A | 659 | −18.125 | 4.788 | 18.639 | 1.00 | 16.93 |
| ATOM | 981 | CA | GLY | A | 659 | −18.500 | 4.360 | 19.972 | 1.00 | 18.25 |
| ATOM | 982 | C | GLY | A | 659 | −19.458 | 3.189 | 20.003 | 1.00 | 18.14 |
| ATOM | 983 | O | GLY | A | 659 | −19.993 | 2.857 | 21.060 | 1.00 | 17.96 |
| ATOM | 984 | N | VAL | A | 660 | −19.695 | 2.581 | 18.844 | 1.00 | 19.22 |
| ATOM | 985 | CA | VAL | A | 660 | −20.573 | 1.418 | 18.749 | 1.00 | 20.26 |
| ATOM | 986 | CB | VAL | A | 660 | −21.909 | 1.771 | 18.060 | 1.00 | 19.84 |
| ATOM | 987 | CG1 | VAL | A | 660 | −22.697 | 2.730 | 18.941 | 1.00 | 20.64 |
| ATOM | 988 | CG2 | VAL | A | 660 | −21.655 | 2.391 | 16.698 | 1.00 | 20.17 |
| ATOM | 989 | C | VAL | A | 660 | −19.844 | 0.334 | 17.958 | 1.00 | 20.35 |
| ATOM | 990 | O | VAL | A | 660 | −18.953 | 0.630 | 17.160 | 1.00 | 20.64 |
| ATOM | 991 | N | SER | A | 661 | −20.226 | −0.915 | 18.190 | 1.00 | 18.66 |
| ATOM | 992 | CA | SER | A | 661 | −19.605 | −2.069 | 17.541 | 1.00 | 19.40 |
| ATOM | 993 | CB | SER | A | 661 | −20.001 | −3.341 | 18.278 | 1.00 | 23.46 |
| ATOM | 994 | OG | SER | A | 661 | −21.369 | −3.622 | 18.044 | 1.00 | 22.76 |
| ATOM | 995 | C | SER | A | 661 | −19.955 | −2.260 | 16.069 | 1.00 | 19.19 |
| ATOM | 996 | O | SER | A | 661 | −20.857 | −1.614 | 15.539 | 1.00 | 19.47 |
| ATOM | 997 | N | ASN | A | 662 | −19.237 | −3.175 | 15.421 | 1.00 | 20.96 |
| ATOM | 998 | CA | ASN | A | 662 | −19.493 | −3.489 | 14.018 | 1.00 | 21.17 |
| ATOM | 999 | CB | ASN | A | 662 | −18.474 | −4.500 | 13.487 | 1.00 | 20.86 |
| ATOM | 1000 | CG | ASN | A | 662 | −17.189 | −3.844 | 13.053 | 1.00 | 20.37 |
| ATOM | 1001 | OD1 | ASN | A | 662 | −17.200 | −2.702 | 12.606 | 1.00 | 21.25 |
| ATOM | 1002 | ND2 | ASN | A | 662 | −16.078 | −4.566 | 13.158 | 1.00 | 19.75 |
| ATOM | 1003 | C | ASN | A | 662 | −20.894 | −4.065 | 13.895 | 1.00 | 21.10 |
| ATOM | 1004 | O | ASN | A | 662 | −21.651 | −3.713 | 12.985 | 1.00 | 21.05 |
| ATOM | 1005 | N | GLN | A | 663 | −21.242 | −4.941 | 14.831 | 1.00 | 25.13 |
| ATOM | 1006 | CA | GLN | A | 663 | −22.556 | −5.555 | 14.836 | 1.00 | 26.08 |
| ATOM | 1007 | CB | GLN | A | 663 | −22.696 | −6.503 | 16.031 | 1.00 | 35.28 |
| ATOM | 1008 | CG | GLN | A | 663 | −21.884 | −7.790 | 15.904 | 1.00 | 36.63 |
| ATOM | 1009 | CD | GLN | A | 663 | −20.381 | −7.582 | 16.043 | 1.00 | 36.48 |
| ATOM | 1010 | OE1 | GLN | A | 663 | −19.588 | −8.447 | 15.665 | 1.00 | 36.95 |
| ATOM | 1011 | NE2 | GLN | A | 663 | −19.983 | −6.441 | 16.600 | 1.00 | 36.26 |
| ATOM | 1012 | C | GLN | A | 663 | −23.638 | −4.480 | 14.881 | 1.00 | 25.84 |
| ATOM | 1013 | O | GLN | A | 663 | −24.625 | −4.554 | 14.153 | 1.00 | 25.82 |
| ATOM | 1014 | N | PHE | A | 664 | −23.451 | −3.474 | 15.730 | 1.00 | 24.41 |
| ATOM | 1015 | CA | PHE | A | 664 | −24.423 | −2.394 | 15.832 | 1.00 | 25.34 |
| ATOM | 1016 | CB | PHE | A | 664 | −23.991 | −1.391 | 16.906 | 1.00 | 26.39 |
| ATOM | 1017 | CG | PHE | A | 664 | −24.932 | −0.226 | 17.069 | 1.00 | 27.42 |
| ATOM | 1018 | CD1 | PHE | A | 664 | −24.841 | 0.881 | 16.230 | 1.00 | 26.41 |
| ATOM | 1019 | CD2 | PHE | A | 664 | −25.915 | −0.240 | 18.057 | 1.00 | 27.03 |
| ATOM | 1020 | CE1 | PHE | A | 664 | −25.713 | 1.963 | 16.367 | 1.00 | 27.10 |
| ATOM | 1021 | CE2 | PHE | A | 664 | −26.795 | 0.840 | 18.202 | 1.00 | 27.63 |
| ATOM | 1022 | CZ | PHE | A | 664 | −26.687 | 1.943 | 17.350 | 1.00 | 26.59 |
| ATOM | 1023 | C | PHE | A | 664 | −24.577 | −1.697 | 14.476 | 1.00 | 25.16 |
| ATOM | 1024 | O | PHE | A | 664 | −25.691 | −1.485 | 14.006 | 1.00 | 24.58 |
| ATOM | 1025 | N | LEU | A | 665 | −23.454 | −1.361 | 13.847 | 1.00 | 21.05 |
| ATOM | 1026 | CA | LEU | A | 665 | −23.479 | −0.698 | 12.542 | 1.00 | 22.93 |
| ATOM | 1027 | CB | LEU | A | 665 | −22.058 | −0.348 | 12.104 | 1.00 | 22.29 |
| ATOM | 1028 | CG | LEU | A | 665 | −21.328 | 0.745 | 12.893 | 1.00 | 22.66 |
| ATOM | 1029 | CD1 | LEU | A | 665 | −19.914 | 0.890 | 12.347 | 1.00 | 22.68 |
| ATOM | 1030 | CD2 | LEU | A | 665 | −22.069 | 2.064 | 12.776 | 1.00 | 22.45 |
| ATOM | 1031 | C | LEU | A | 665 | −24.147 | −1.563 | 11.471 | 1.00 | 22.68 |
| ATOM | 1032 | O | LEU | A | 665 | −24.748 | −1.053 | 10.520 | 1.00 | 23.83 |
| ATOM | 1033 | N | ILE | A | 666 | −24.031 | −2.874 | 11.618 | 1.00 | 24.12 |
| ATOM | 1034 | CA | ILE | A | 666 | −24.635 | −3.794 | 10.664 | 1.00 | 24.57 |
| ATOM | 1035 | CB | ILE | A | 666 | −24.023 | −5.204 | 10.785 | 1.00 | 26.67 |
| ATOM | 1036 | CG2 | ILE | A | 666 | −24.819 | −6.195 | 9.951 | 1.00 | 26.86 |
| ATOM | 1037 | CG1 | ILE | A | 666 | −22.559 | −5.176 | 10.340 | 1.00 | 25.51 |
| ATOM | 1038 | CD1 | ILE | A | 666 | −21.811 | −6.483 | 10.553 | 1.00 | 26.41 |
| ATOM | 1039 | C | ILE | A | 666 | −26.136 | −3.880 | 10.926 | 1.00 | 26.29 |
| ATOM | 1040 | O | ILE | A | 666 | −26.937 | −3.859 | 9.993 | 1.00 | 26.10 |
| ATOM | 1041 | N | ASN | A | 667 | −26.511 | −3.968 | 12.197 | 1.00 | 31.89 |
| ATOM | 1042 | CA | ASN | A | 667 | −27.920 | −4.070 | 12.563 | 1.00 | 34.09 |
| ATOM | 1043 | CB | ASN | A | 667 | −28.063 | −4.437 | 14.044 | 1.00 | 34.18 |
| ATOM | 1044 | CG | ASN | A | 667 | −27.511 | −5.809 | 14.355 | 1.00 | 33.95 |
| ATOM | 1045 | OD1 | ASN | A | 667 | −27.608 | −6.722 | 13.538 | 1.00 | 34.50 |
| ATOM | 1046 | ND2 | ASN | A | 667 | −26.939 | −5.969 | 15.544 | 1.00 | 34.36 |
| ATOM | 1047 | C | ASN | A | 667 | −28.733 | −2.816 | 12.270 | 1.00 | 34.67 |
| ATOM | 1048 | O | ASN | A | 667 | −29.944 | −2.891 | 12.058 | 1.00 | 34.88 |
| ATOM | 1049 | N | THR | A | 668 | −28.073 | −1.665 | 12.264 | 1.00 | 41.41 |
| ATOM | 1050 | CA | THR | A | 668 | −28.755 | −0.408 | 11.993 | 1.00 | 41.66 |
| ATOM | 1051 | CB | THR | A | 668 | −28.180 | 0.728 | 12.851 | 1.00 | 34.82 |
| ATOM | 1052 | OG1 | THR | A | 668 | −26.774 | 0.855 | 12.598 | 1.00 | 34.37 |
| ATOM | 1053 | CG2 | THR | A | 668 | −28.404 | 0.438 | 14.325 | 1.00 | 34.77 |
| ATOM | 1054 | C | THR | A | 668 | −28.630 | −0.028 | 10.521 | 1.00 | 41.72 |
| ATOM | 1055 | O | THR | A | 668 | −29.012 | 1.070 | 10.121 | 1.00 | 42.66 |
| ATOM | 1056 | N | ASN | A | 669 | −28.096 | −0.945 | 9.720 | 1.00 | 36.50 |
| ATOM | 1057 | CA | ASN | A | 669 | −27.916 | −0.715 | 8.289 | 1.00 | 35.43 |
| ATOM | 1058 | CB | ASN | A | 669 | −29.271 | −0.756 | 7.581 | 1.00 | 39.66 |
| ATOM | 1059 | CG | ASN | A | 669 | −29.861 | −2.147 | 7.553 | 1.00 | 39.15 |
| ATOM | 1060 | OD1 | ASN | A | 669 | −29.314 | −3.048 | 6.919 | 1.00 | 39.61 |
| ATOM | 1061 | ND2 | ASN | A | 669 | −30.974 | −2.334 | 8.247 | 1.00 | 39.97 |
| ATOM | 1062 | C | ASN | A | 669 | −27.217 | 0.603 | 8.002 | 1.00 | 33.95 |
| ATOM | 1063 | O | ASN | A | 669 | −27.676 | 1.406 | 7.187 | 1.00 | 35.22 |
| ATOM | 1064 | N | SER | A | 670 | −26.098 | 0.816 | 8.684 | 1.00 | 24.12 |
| ATOM | 1065 | CA | SER | A | 670 | −25.304 | 2.020 | 8.524 | 1.00 | 24.45 |
| ATOM | 1066 | CB | SER | A | 670 | −24.162 | 2.032 | 9.543 | 1.00 | 36.85 |
| ATOM | 1067 | OG | SER | A | 670 | −23.245 | 3.081 | 9.279 | 1.00 | 38.12 |
| ATOM | 1068 | C | SER | A | 670 | −24.727 | 2.056 | 7.120 | 1.00 | 22.19 |
| ATOM | 1069 | O | SER | A | 670 | −24.529 | 1.013 | 6.498 | 1.00 | 22.80 |
| ATOM | 1070 | N | GLU | A | 671 | −24.461 | 3.261 | 6.626 | 1.00 | 25.15 |
| ATOM | 1071 | CA | GLU | A | 671 | −23.888 | 3.426 | 5.299 | 1.00 | 25.09 |
| ATOM | 1072 | CB | GLU | A | 671 | −23.736 | 4.911 | 4.975 | 1.00 | 30.03 |
| ATOM | 1073 | CG | GLU | A | 671 | −23.322 | 5.198 | 3.547 | 1.00 | 30.00 |
| ATOM | 1074 | CD | GLU | A | 671 | −23.174 | 6.683 | 3.277 | 1.00 | 30.78 |
| ATOM | 1075 | OE1 | GLU | A | 671 | −22.113 | 7.253 | 3.613 | 1.00 | 30.04 |
| ATOM | 1076 | OE2 | GLU | A | 671 | −24.132 | 7.283 | 2.742 | 1.00 | 30.29 |
| ATOM | 1077 | C | GLU | A | 671 | −22.521 | 2.757 | 5.281 | 1.00 | 23.99 |
| ATOM | 1078 | O | GLU | A | 671 | −22.087 | 2.222 | 4.267 | 1.00 | 24.80 |
| ATOM | 1079 | N | LEU | A | 672 | −21.857 | 2.786 | 6.429 | 1.00 | 23.33 |
| ATOM | 1080 | CA | LEU | A | 672 | −20.533 | 2.196 | 6.581 | 1.00 | 21.52 |
| ATOM | 1081 | CB | LEU | A | 672 | −20.010 | 2.512 | 7.984 | 1.00 | 30.40 |
| ATOM | 1082 | CG | LEU | A | 672 | −18.683 | 3.260 | 8.158 | 1.00 | 31.15 |
| ATOM | 1083 | CD1 | LEU | A | 672 | −18.555 | 4.398 | 7.164 | 1.00 | 30.73 |
| ATOM | 1084 | CD2 | LEU | A | 672 | −18.603 | 3.772 | 9.586 | 1.00 | 31.17 |
| ATOM | 1085 | C | LEU | A | 672 | −20.561 | 0.685 | 6.349 | 1.00 | 22.19 |
| ATOM | 1086 | O | LEU | A | 672 | −19.688 | 0.138 | 5.673 | 1.00 | 20.56 |
| ATOM | 1087 | N | ALA | A | 673 | −21.565 | 0.014 | 6.910 | 1.00 | 20.08 |
| ATOM | 1088 | CA | ALA | A | 673 | −21.685 | −1.434 | 6.766 | 1.00 | 20.23 |
| ATOM | 1089 | CB | ALA | A | 673 | −22.827 | −1.958 | 7.639 | 1.00 | 23.81 |
| ATOM | 1090 | C | ALA | A | 673 | −21.934 | −1.794 | 5.310 | 1.00 | 20.27 |
| ATOM | 1091 | O | ALA | A | 673 | −21.446 | −2.808 | 4.821 | 1.00 | 20.29 |
| ATOM | 1092 | N | LEU | A | 674 | −22.714 | −0.970 | 4.620 | 1.00 | 18.83 |
| ATOM | 1093 | CA | LEU | A | 674 | −22.998 | −1.229 | 3.213 | 1.00 | 20.25 |
| ATOM | 1094 | CB | LEU | A | 674 | −24.063 | −0.265 | 2.694 | 1.00 | 26.85 |
| ATOM | 1095 | CG | LEU | A | 674 | −24.330 | −0.385 | 1.189 | 1.00 | 27.73 |
| ATOM | 1096 | CD1 | LEU | A | 674 | −24.803 | −1.793 | 0.849 | 1.00 | 27.05 |
| ATOM | 1097 | CD2 | LEU | A | 674 | −25.364 | 0.646 | 0.779 | 1.00 | 27.39 |
| ATOM | 1098 | C | LEU | A | 674 | −21.740 | −1.082 | 2.363 | 1.00 | 18.25 |
| ATOM | 1099 | O | LEU | A | 674 | −21.454 | −1.915 | 1.500 | 1.00 | 21.42 |
| ATOM | 1100 | N | MET | A | 675 | −20.980 | −0.027 | 2.624 | 1.00 | 22.09 |
| ATOM | 1101 | CA | MET | A | 675 | −19.758 | 0.244 | 1.874 | 1.00 | 23.03 |
| ATOM | 1102 | CB | MET | A | 675 | −19.140 | 1.562 | 2.330 | 1.00 | 41.71 |
| ATOM | 1103 | CG | MET | A | 675 | −20.013 | 2.771 | 2.096 | 1.00 | 42.43 |
| ATOM | 1104 | SD | MET | A | 675 | −19.158 | 4.258 | 2.607 | 1.00 | 43.58 |
| ATOM | 1105 | CE | MET | A | 675 | −18.192 | 4.598 | 1.138 | 1.00 | 42.87 |
| ATOM | 1106 | C | MET | A | 675 | −18.711 | −0.848 | 2.018 | 1.00 | 23.59 |
| ATOM | 1107 | O | MET | A | 675 | −17.961 | −1.130 | 1.079 | 1.00 | 22.39 |
| ATOM | 1108 | N | TYR | A | 676 | −18.662 | −1.463 | 3.193 | 1.00 | 22.64 |
| ATOM | 1109 | CA | TYR | A | 676 | −17.670 | −2.495 | 3.451 | 1.00 | 22.20 |
| ATOM | 1110 | CB | TYR | A | 676 | −16.894 | −2.113 | 4.717 | 1.00 | 21.16 |
| ATOM | 1111 | CG | TYR | A | 676 | −16.197 | −0.767 | 4.574 | 1.00 | 21.36 |
| ATOM | 1112 | CD1 | TYR | A | 676 | −16.385 | 0.249 | 5.513 | 1.00 | 21.19 |
| ATOM | 1113 | CE1 | TYR | A | 676 | −15.774 | 1.501 | 5.352 | 1.00 | 20.94 |
| ATOM | 1114 | CD2 | TYR | A | 676 | −15.378 | −0.503 | 3.475 | 1.00 | 20.92 |
| ATOM | 1115 | CE2 | TYR | A | 676 | −14.760 | 0.739 | 3.307 | 1.00 | 20.99 |
| ATOM | 1116 | CZ | TYR | A | 676 | −14.964 | 1.734 | 4.242 | 1.00 | 21.41 |
| ATOM | 1117 | OH | TYR | A | 676 | −14.374 | 2.969 | 4.059 | 1.00 | 22.70 |
| ATOM | 1118 | C | TYR | A | 676 | −18.206 | −3.926 | 3.531 | 1.00 | 23.64 |
| ATOM | 1119 | O | TYR | A | 676 | −17.519 | −4.824 | 4.019 | 1.00 | 22.37 |
| ATOM | 1120 | N | ASN | A | 677 | −19.421 | −4.129 | 3.025 | 1.00 | 21.83 |
| ATOM | 1121 | CA | ASN | A | 677 | −20.071 | −5.444 | 2.995 | 1.00 | 23.13 |
| ATOM | 1122 | CB | ASN | A | 677 | −19.487 | −6.296 | 1.861 | 1.00 | 56.74 |
| ATOM | 1123 | CG | ASN | A | 677 | −18.007 | −6.571 | 2.033 | 1.00 | 57.80 |
| ATOM | 1124 | OD1 | ASN | A | 677 | −17.594 | −7.241 | 2.979 | 1.00 | 58.17 |
| ATOM | 1125 | ND2 | ASN | A | 677 | −17.197 | −6.052 | 1.116 | 1.00 | 58.15 |
| ATOM | 1126 | C | ASN | A | 677 | −20.019 | −6.223 | 4.311 | 1.00 | 22.92 |
| ATOM | 1127 | O | ASN | A | 677 | −19.726 | −7.425 | 4.332 | 1.00 | 22.51 |
| ATOM | 1128 | N | ASP | A | 678 | −20.317 | −5.521 | 5.398 | 1.00 | 22.36 |
| ATOM | 1129 | CA | ASP | A | 678 | −20.353 | −6.096 | 6.742 | 1.00 | 23.31 |
| ATOM | 1130 | CB | ASP | A | 678 | −21.461 | −7.146 | 6.843 | 1.00 | 24.85 |
| ATOM | 1131 | CG | ASP | A | 678 | −22.824 | −6.604 | 6.466 | 1.00 | 25.27 |
| ATOM | 1132 | OD1 | ASP | A | 678 | −23.031 | −5.376 | 6.536 | 1.00 | 23.89 |
| ATOM | 1133 | OD2 | ASP | A | 678 | −23.699 | −7.420 | 6.111 | 1.00 | 26.42 |
| ATOM | 1134 | C | ASP | A | 678 | −19.070 | −6.725 | 7.261 | 1.00 | 21.81 |
| ATOM | 1135 | O | ASP | A | 678 | −19.084 | −7.336 | 8.332 | 1.00 | 23.49 |
| ATOM | 1136 | N | GLU | A | 679 | −17.972 | −6.578 | 6.533 | 1.00 | 19.14 |
| ATOM | 1137 | CA | GLU | A | 679 | −16.703 | −7.166 | 6.962 | 1.00 | 20.71 |
| ATOM | 1138 | CB | GLU | A | 679 | −15.988 | −7.819 | 5.778 | 1.00 | 46.03 |
| ATOM | 1139 | CG | GLU | A | 679 | −16.640 | −9.084 | 5.259 | 1.00 | 47.01 |
| ATOM | 1140 | CD | GLU | A | 679 | −15.840 | −9.724 | 4.139 | 1.00 | 47.28 |
| ATOM | 1141 | OE1 | GLU | A | 679 | −15.723 | −9.106 | 3.059 | 1.00 | 47.36 |
| ATOM | 1142 | OE2 | GLU | A | 679 | −15.320 | −10.841 | 4.337 | 1.00 | 47.19 |
| ATOM | 1143 | C | GLU | A | 679 | −15.770 | −6.137 | 7.582 | 1.00 | 19.51 |
| ATOM | 1144 | O | GLU | A | 679 | −15.378 | −5.185 | 6.912 | 1.00 | 20.45 |
| ATOM | 1145 | N | SER | A | 680 | −15.407 | −6.345 | 8.848 | 1.00 | 17.83 |
| ATOM | 1146 | CA | SER | A | 680 | −14.505 | −5.442 | 9.574 | 1.00 | 17.23 |
| ATOM | 1147 | CB | SER | A | 680 | −13.043 | −5.747 | 9.224 | 1.00 | 21.15 |
| ATOM | 1148 | OG | SER | A | 680 | −12.707 | −7.098 | 9.511 | 1.00 | 20.61 |
| ATOM | 1149 | C | SER | A | 680 | −14.827 | −3.997 | 9.202 | 1.00 | 16.25 |
| ATOM | 1150 | O | SER | A | 680 | −13.941 | −3.218 | 8.844 | 1.00 | 15.64 |
| ATOM | 1151 | N | VAL | A | 681 | −16.108 | −3.664 | 9.309 | 1.00 | 18.34 |
| ATOM | 1152 | CA | VAL | A | 681 | −16.629 | −2.353 | 8.947 | 1.00 | 16.30 |
| ATOM | 1153 | CB | VAL | A | 681 | −18.115 | −2.245 | 9.327 | 1.00 | 19.28 |
| ATOM | 1154 | CG1 | VAL | A | 681 | −18.674 | −0.895 | 8.881 | 1.00 | 20.72 |
| ATOM | 1155 | CG2 | VAL | A | 681 | −18.894 | −3.397 | 8.665 | 1.00 | 19.24 |
| ATOM | 1156 | C | VAL | A | 681 | −15.864 | −1.173 | 9.529 | 1.00 | 16.04 |
| ATOM | 1157 | O | VAL | A | 681 | −15.356 | −0.347 | 8.790 | 1.00 | 17.05 |
| ATOM | 1158 | N | LEU | A | 681A | −15.782 | −1.110 | 10.852 | 1.00 | 15.07 |
| ATOM | 1159 | CA | LEU | A | 681A | −15.079 | −0.028 | 11.529 | 1.00 | 16.07 |
| ATOM | 1160 | CB | LEU | A | 681A | −15.166 | −0.217 | 13.043 | 1.00 | 22.39 |
| ATOM | 1161 | CG | LEU | A | 681A | −16.520 | 0.073 | 13.680 | 1.00 | 24.40 |
| ATOM | 1162 | CD1 | LEU | A | 681A | −16.571 | −0.536 | 15.076 | 1.00 | 24.68 |
| ATOM | 1163 | CD2 | LEU | A | 681A | −16.732 | 1.573 | 13.710 | 1.00 | 25.32 |
| ATOM | 1164 | C | LEU | A | 681A | −13.613 | 0.027 | 11.125 | 1.00 | 15.73 |
| ATOM | 1165 | O | LEU | A | 681A | −13.070 | 1.089 | 10.848 | 1.00 | 14.66 |
| ATOM | 1166 | N | GLU | A | 682 | −12.975 | −1.134 | 11.078 | 1.00 | 14.20 |
| ATOM | 1167 | CA | GLU | A | 682 | −11.566 | −1.181 | 10.731 | 1.00 | 16.26 |
| ATOM | 1168 | CB | GLU | A | 682 | −11.042 | −2.601 | 10.954 | 1.00 | 17.70 |
| ATOM | 1169 | CG | GLU | A | 682 | −11.075 | −3.043 | 12.424 | 1.00 | 17.27 |
| ATOM | 1170 | CD | GLU | A | 682 | −12.466 | −3.381 | 12.956 | 1.00 | 17.63 |
| ATOM | 1171 | OE1 | GLU | A | 682 | −13.350 | −3.778 | 12.164 | 1.00 | 16.51 |
| ATOM | 1172 | OE2 | GLU | A | 682 | −12.672 | −3.273 | 14.192 | 1.00 | 18.25 |
| ATOM | 1173 | C | GLU | A | 682 | −11.272 | −0.675 | 9.313 | 1.00 | 13.40 |
| ATOM | 1174 | O | GLU | A | 682 | −10.257 | −0.010 | 9.088 | 1.00 | 16.15 |
| ATOM | 1175 | N | HIS | A | 683 | −12.140 | −0.983 | 8.353 | 1.00 | 14.40 |
| ATOM | 1176 | CA | HIS | A | 683 | −11.943 | −0.474 | 7.001 | 1.00 | 15.72 |
| ATOM | 1177 | CB | HIS | A | 683 | −12.955 | −1.084 | 6.030 | 1.00 | 20.22 |
| ATOM | 1178 | CG | HIS | A | 683 | −12.527 | −2.406 | 5.483 | 1.00 | 21.36 |
| ATOM | 1179 | CD2 | HIS | A | 683 | −12.905 | −3.667 | 5.800 | 1.00 | 22.11 |
| ATOM | 1180 | ND1 | HIS | A | 683 | −11.538 | −2.529 | 4.531 | 1.00 | 19.70 |
| ATOM | 1181 | CE1 | HIS | A | 683 | −11.325 | −3.810 | 4.286 | 1.00 | 20.00 |
| ATOM | 1182 | NE2 | HIS | A | 683 | −12.139 | −4.520 | 5.044 | 1.00 | 21.26 |
| ATOM | 1183 | C | HIS | A | 683 | −12.102 | 1.046 | 7.044 | 1.00 | 15.07 |
| ATOM | 1184 | O | HIS | A | 683 | −11.395 | 1.772 | 6.343 | 1.00 | 16.13 |
| ATOM | 1185 | N | HIS | A | 684 | −13.033 | 1.519 | 7.874 | 1.00 | 16.25 |
| ATOM | 1186 | CA | HIS | A | 684 | −13.258 | 2.952 | 8.026 | 1.00 | 17.73 |
| ATOM | 1187 | CB | HIS | A | 684 | −14.505 | 3.216 | 8.878 | 1.00 | 19.40 |
| ATOM | 1188 | CG | HIS | A | 684 | −14.776 | 4.670 | 9.116 | 1.00 | 20.37 |
| ATOM | 1189 | CD2 | HIS | A | 684 | −14.870 | 5.387 | 10.261 | 1.00 | 19.50 |
| ATOM | 1190 | ND1 | HIS | A | 684 | −14.982 | 5.563 | 8.087 | 1.00 | 20.04 |
| ATOM | 1191 | CE1 | HIS | A | 684 | −15.194 | 6.766 | 8.588 | 1.00 | 19.05 |
| ATOM | 1192 | NE2 | HIS | A | 684 | −15.131 | 6.688 | 9.903 | 1.00 | 18.64 |
| ATOM | 1193 | C | HIS | A | 684 | −12.038 | 3.601 | 8.677 | 1.00 | 17.41 |
| ATOM | 1194 | O | HIS | A | 684 | −11.602 | 4.677 | 8.248 | 1.00 | 17.52 |
| ATOM | 1195 | N | HIS | A | 685 | −11.491 | 2.952 | 9.704 | 1.00 | 16.56 |
| ATOM | 1196 | CA | HIS | A | 685 | −10.316 | 3.481 | 10.387 | 1.00 | 17.88 |
| ATOM | 1197 | CB | HIS | A | 685 | −9.886 | 2.568 | 11.553 | 1.00 | 17.97 |
| ATOM | 1198 | CG | HIS | A | 685 | −10.943 | 2.365 | 12.599 | 1.00 | 17.64 |
| ATOM | 1199 | CD2 | HIS | A | 685 | −11.311 | 1.258 | 13.291 | 1.00 | 17.69 |
| ATOM | 1200 | ND1 | HIS | A | 685 | −11.728 | 3.392 | 13.079 | 1.00 | 16.45 |
| ATOM | 1201 | CE1 | HIS | A | 685 | −12.533 | 2.927 | 14.020 | 1.00 | 18.14 |
| ATOM | 1202 | NE2 | HIS | A | 685 | −12.299 | 1.634 | 14.168 | 1.00 | 17.23 |
| ATOM | 1203 | C | HIS | A | 685 | −9.178 | 3.619 | 9.380 | 1.00 | 17.11 |
| ATOM | 1204 | O | HIS | A | 685 | −8.482 | 4.639 | 9.348 | 1.00 | 17.09 |
| ATOM | 1205 | N | PHE | A | 686 | −8.984 | 2.596 | 8.551 | 1.00 | 17.41 |
| ATOM | 1206 | CA | PHE | A | 686 | −7.928 | 2.656 | 7.551 | 1.00 | 17.43 |
| ATOM | 1207 | CB | PHE | A | 686 | −7.766 | 1.325 | 6.810 | 1.00 | 22.00 |
| ATOM | 1208 | CG | PHE | A | 686 | −6.738 | 1.384 | 5.715 | 1.00 | 21.91 |
| ATOM | 1209 | CD1 | PHE | A | 686 | −5.396 | 1.591 | 6.014 | 1.00 | 22.44 |
| ATOM | 1210 | CD2 | PHE | A | 686 | −7.123 | 1.328 | 4.379 | 1.00 | 22.63 |
| ATOM | 1211 | CE1 | PHE | A | 686 | −4.449 | 1.754 | 4.998 | 1.00 | 21.95 |
| ATOM | 1212 | CE2 | PHE | A | 686 | −6.186 | 1.487 | 3.356 | 1.00 | 22.74 |
| ATOM | 1213 | CZ | PHE | A | 686 | −4.847 | 1.704 | 3.668 | 1.00 | 23.18 |
| ATOM | 1214 | C | PHE | A | 686 | −8.183 | 3.760 | 6.531 | 1.00 | 18.51 |
| ATOM | 1215 | O | PHE | A | 686 | −7.250 | 4.436 | 6.104 | 1.00 | 18.35 |
| ATOM | 1216 | N | ASP | A | 687 | −9.439 | 3.934 | 6.130 | 1.00 | 17.80 |
| ATOM | 1217 | CA | ASP | A | 687 | −9.768 | 4.974 | 5.166 | 1.00 | 18.73 |
| ATOM | 1218 | CB | ASP | A | 687 | −11.262 | 4.969 | 4.836 | 1.00 | 27.45 |
| ATOM | 1219 | CG | ASP | A | 687 | −11.661 | 6.129 | 3.932 | 1.00 | 29.42 |
| ATOM | 1220 | OD1 | ASP | A | 687 | −11.162 | 6.190 | 2.789 | 1.00 | 29.29 |
| ATOM | 1221 | OD2 | ASP | A | 687 | −12.465 | 6.984 | 4.366 | 1.00 | 29.84 |
| ATOM | 1222 | C | ASP | A | 687 | −9.389 | 6.329 | 5.751 | 1.00 | 17.84 |
| ATOM | 1223 | O | ASP | A | 687 | −8.793 | 7.164 | 5.070 | 1.00 | 18.06 |
| ATOM | 1224 | N | GLN | A | 688 | −9.726 | 6.534 | 7.020 | 1.00 | 20.35 |
| ATOM | 1225 | CA | GLN | A | 688 | −9.412 | 7.786 | 7.703 | 1.00 | 19.96 |
| ATOM | 1226 | CB | GLN | A | 688 | −10.060 | 7.804 | 9.085 | 1.00 | 24.85 |
| ATOM | 1227 | CG | GLN | A | 688 | −11.577 | 7.795 | 9.021 | 1.00 | 25.62 |
| ATOM | 1228 | CD | GLN | A | 688 | −12.120 | 9.081 | 8.440 | 1.00 | 26.01 |
| ATOM | 1229 | OE1 | GLN | A | 688 | −12.052 | 10.129 | 9.076 | 1.00 | 27.97 |
| ATOM | 1230 | NE2 | GLN | A | 688 | −12.647 | 9.012 | 7.221 | 1.00 | 27.26 |
| ATOM | 1231 | C | GLN | A | 688 | −7.911 | 7.969 | 7.831 | 1.00 | 20.01 |
| ATOM | 1232 | O | GLN | A | 688 | −7.406 | 9.077 | 7.689 | 1.00 | 19.66 |
| ATOM | 1233 | N | CYS | A | 689 | −7.202 | 6.871 | 8.091 | 1.00 | 18.42 |
| ATOM | 1234 | CA | CYS | A | 689 | −5.746 | 6.902 | 8.229 | 1.00 | 18.66 |
| ATOM | 1235 | CB | CYS | A | 689 | −5.224 | 5.498 | 8.556 | 1.00 | 19.50 |
| ATOM | 1236 | SG | CYS | A | 689 | −3.425 | 5.370 | 8.637 | 1.00 | 16.97 |
| ATOM | 1237 | C | CYS | A | 689 | −5.098 | 7.413 | 6.942 | 1.00 | 18.50 |
| ATOM | 1238 | O | CYS | A | 689 | −4.234 | 8.295 | 6.977 | 1.00 | 19.39 |
| ATOM | 1239 | N | LEU | A | 690 | −5.524 | 6.854 | 5.812 | 1.00 | 19.82 |
| ATOM | 1240 | CA | LEU | A | 690 | −5.006 | 7.235 | 4.497 | 1.00 | 20.55 |
| ATOM | 1241 | CB | LEU | A | 690 | −5.556 | 6.288 | 3.429 | 1.00 | 25.80 |
| ATOM | 1242 | CG | LEU | A | 690 | −4.985 | 6.446 | 2.017 | 1.00 | 26.79 |
| ATOM | 1243 | CD1 | LEU | A | 690 | −3.507 | 6.105 | 2.029 | 1.00 | 27.37 |
| ATOM | 1244 | CD2 | LEU | A | 690 | −5.728 | 5.535 | 1.059 | 1.00 | 27.51 |
| ATOM | 1245 | C | LEU | A | 690 | −5.389 | 8.678 | 4.148 | 1.00 | 19.76 |
| ATOM | 1246 | O | LEU | A | 690 | −4.621 | 9.401 | 3.521 | 1.00 | 21.37 |
| ATOM | 1247 | N | MET | A | 691 | −6.587 | 9.088 | 4.549 | 1.00 | 21.76 |
| ATOM | 1248 | CA | MET | A | 691 | −7.044 | 10.451 | 4.285 | 1.00 | 21.56 |
| ATOM | 1249 | CB | MET | A | 691 | −8.450 | 10.654 | 4.851 | 1.00 | 39.93 |
| ATOM | 1250 | CG | MET | A | 691 | −9.006 | 12.052 | 4.633 | 1.00 | 40.74 |
| ATOM | 1251 | SD | MET | A | 691 | −10.617 | 12.278 | 5.400 | 1.00 | 42.16 |
| ATOM | 1252 | CE | MET | A | 691 | −10.138 | 12.715 | 7.088 | 1.00 | 40.97 |
| ATOM | 1253 | C | MET | A | 691 | −6.079 | 11.451 | 4.928 | 1.00 | 22.42 |
| ATOM | 1254 | O | MET | A | 691 | −5.673 | 12.435 | 4.300 | 1.00 | 21.69 |
| ATOM | 1255 | N | ILE | A | 692 | −5.712 | 11.200 | 6.179 | 1.00 | 20.61 |
| ATOM | 1256 | CA | ILE | A | 692 | −4.799 | 12.094 | 6.878 | 1.00 | 21.43 |
| ATOM | 1257 | CB | ILE | A | 692 | −4.704 | 11.744 | 8.385 | 1.00 | 23.64 |
| ATOM | 1258 | CG2 | ILE | A | 692 | −3.670 | 12.633 | 9.066 | 1.00 | 23.04 |
| ATOM | 1259 | CG1 | ILE | A | 692 | −6.072 | 11.921 | 9.056 | 1.00 | 23.49 |
| ATOM | 1260 | CD1 | ILE | A | 692 | −6.591 | 13.352 | 9.054 | 1.00 | 25.57 |
| ATOM | 1261 | C | ILE | A | 692 | −3.403 | 12.045 | 6.252 | 1.00 | 22.50 |
| ATOM | 1262 | O | ILE | A | 692 | −2.759 | 13.081 | 6.084 | 1.00 | 22.01 |
| ATOM | 1263 | N | LEU | A | 693 | −2.940 | 10.846 | 5.901 | 1.00 | 24.42 |
| ATOM | 1264 | CA | LEU | A | 693 | −1.615 | 10.689 | 5.298 | 1.00 | 25.47 |
| ATOM | 1265 | CB | LEU | A | 693 | −1.288 | 9.203 | 5.087 | 1.00 | 21.69 |
| ATOM | 1266 | CG | LEU | A | 693 | −0.950 | 8.368 | 6.333 | 1.00 | 21.16 |
| ATOM | 1267 | CD1 | LEU | A | 693 | −0.945 | 6.888 | 5.985 | 1.00 | 22.43 |
| ATOM | 1268 | CD2 | LEU | A | 693 | 0.411 | 8.794 | 6.887 | 1.00 | 21.71 |
| ATOM | 1269 | C | LEU | A | 693 | −1.499 | 11.437 | 3.972 | 1.00 | 25.25 |
| ATOM | 1270 | O | LEU | A | 693 | −0.414 | 11.883 | 3.601 | 1.00 | 26.01 |
| ATOM | 1271 | N | ASN | A | 694 | −2.614 | 11.579 | 3.261 | 1.00 | 23.58 |
| ATOM | 1272 | CA | ASN | A | 694 | −2.610 | 12.284 | 1.981 | 1.00 | 22.99 |
| ATOM | 1273 | CB | ASN | A | 694 | −3.613 | 11.656 | 1.005 | 1.00 | 26.66 |
| ATOM | 1274 | CG | ASN | A | 694 | −3.178 | 10.294 | 0.514 | 1.00 | 26.69 |
| ATOM | 1275 | OD1 | ASN | A | 694 | −2.009 | 10.078 | 0.209 | 1.00 | 27.10 |
| ATOM | 1276 | ND2 | ASN | A | 694 | −4.127 | 9.369 | 0.413 | 1.00 | 27.29 |
| ATOM | 1277 | C | ASN | A | 694 | −2.949 | 13.763 | 2.132 | 1.00 | 23.59 |
| ATOM | 1278 | O | ASN | A | 694 | −2.913 | 14.508 | 1.155 | 1.00 | 24.38 |
| ATOM | 1279 | N | SER | A | 695 | −3.285 | 14.181 | 3.348 | 1.00 | 24.35 |
| ATOM | 1280 | CA | SER | A | 695 | −3.644 | 15.572 | 3.596 | 1.00 | 24.87 |
| ATOM | 1281 | CB | SER | A | 695 | −4.279 | 15.727 | 4.980 | 1.00 | 30.85 |
| ATOM | 1282 | OG | SER | A | 695 | −5.570 | 15.146 | 5.009 | 1.00 | 31.53 |
| ATOM | 1283 | C | SER | A | 695 | −2.431 | 16.475 | 3.496 | 1.00 | 25.45 |
| ATOM | 1284 | O | SER | A | 695 | −1.399 | 16.213 | 4.108 | 1.00 | 25.07 |
| ATOM | 1285 | N | PRO | A | 696 | −2.544 | 17.558 | 2.717 | 1.00 | 28.09 |
| ATOM | 1286 | CD | PRO | A | 696 | −3.728 | 18.069 | 2.009 | 1.00 | 32.92 |
| ATOM | 1287 | CA | PRO | A | 696 | −1.410 | 18.471 | 2.579 | 1.00 | 28.93 |
| ATOM | 1288 | CB | PRO | A | 696 | −1.978 | 19.603 | 1.718 | 1.00 | 32.61 |
| ATOM | 1289 | CG | PRO | A | 696 | −3.460 | 19.552 | 1.991 | 1.00 | 33.55 |
| ATOM | 1290 | C | PRO | A | 696 | −0.900 | 18.944 | 3.940 | 1.00 | 28.17 |
| ATOM | 1291 | O | PRO | A | 696 | −1.686 | 19.310 | 4.818 | 1.00 | 27.52 |
| ATOM | 1292 | N | GLY | A | 697 | 0.419 | 18.913 | 4.104 | 1.00 | 27.38 |
| ATOM | 1293 | CA | GLY | A | 697 | 1.035 | 19.336 | 5.347 | 1.00 | 27.26 |
| ATOM | 1294 | C | GLY | A | 697 | 1.106 | 18.239 | 6.391 | 1.00 | 27.36 |
| ATOM | 1295 | O | GLY | A | 697 | 1.688 | 18.430 | 7.460 | 1.00 | 27.28 |
| ATOM | 1296 | N | ASN | A | 698 | 0.523 | 17.084 | 6.080 | 1.00 | 24.05 |
| ATOM | 1297 | CA | ASN | A | 698 | 0.506 | 15.951 | 7.006 | 1.00 | 24.41 |
| ATOM | 1298 | CB | ASN | A | 698 | −0.937 | 15.592 | 7.379 | 1.00 | 26.46 |
| ATOM | 1299 | CG | ASN | A | 698 | −1.624 | 16.668 | 8.190 | 1.00 | 26.12 |
| ATOM | 1300 | OD1 | ASN | A | 698 | −2.038 | 17.702 | 7.664 | 1.00 | 27.64 |
| ATOM | 1301 | ND2 | ASN | A | 698 | −1.749 | 16.428 | 9.486 | 1.00 | 26.42 |
| ATOM | 1302 | C | ASN | A | 698 | 1.165 | 14.698 | 6.436 | 1.00 | 24.62 |
| ATOM | 1303 | O | ASN | A | 698 | 1.157 | 13.643 | 7.076 | 1.00 | 22.80 |
| ATOM | 1304 | N | GLN | A | 699 | 1.734 | 14.812 | 5.240 | 1.00 | 24.05 |
| ATOM | 1305 | CA | GLN | A | 699 | 2.348 | 13.669 | 4.572 | 1.00 | 24.71 |
| ATOM | 1306 | CB | GLN | A | 699 | 2.486 | 13.982 | 3.083 | 1.00 | 37.81 |
| ATOM | 1307 | CG | GLN | A | 699 | 1.165 | 14.399 | 2.451 | 1.00 | 38.52 |
| ATOM | 1308 | CD | GLN | A | 699 | 1.324 | 14.910 | 1.036 | 1.00 | 38.73 |
| ATOM | 1309 | OE1 | GLN | A | 699 | 1.768 | 14.184 | 0.148 | 1.00 | 39.77 |
| ATOM | 1310 | NE2 | GLN | A | 699 | 0.964 | 16.170 | 0.819 | 1.00 | 38.87 |
| ATOM | 1311 | C | GLN | A | 699 | 3.687 | 13.229 | 5.151 | 1.00 | 23.74 |
| ATOM | 1312 | O | GLN | A | 699 | 4.739 | 13.494 | 4.573 | 1.00 | 24.82 |
| ATOM | 1313 | N | ILE | A | 700 | 3.639 | 12.530 | 6.284 | 1.00 | 23.85 |
| ATOM | 1314 | CA | ILE | A | 700 | 4.853 | 12.058 | 6.938 | 1.00 | 23.88 |
| ATOM | 1315 | CB | ILE | A | 700 | 4.572 | 11.572 | 8.374 | 1.00 | 25.21 |
| ATOM | 1316 | CG2 | ILE | A | 700 | 4.101 | 12.736 | 9.225 | 1.00 | 24.98 |
| ATOM | 1317 | CG1 | ILE | A | 700 | 3.528 | 10.454 | 8.363 | 1.00 | 25.64 |
| ATOM | 1318 | CD1 | ILE | A | 700 | 3.370 | 9.764 | 9.699 | 1.00 | 25.16 |
| ATOM | 1319 | C | ILE | A | 700 | 5.550 | 10.935 | 6.182 | 1.00 | 24.95 |
| ATOM | 1320 | O | ILE | A | 700 | 6.662 | 10.540 | 6.546 | 1.00 | 24.94 |
| ATOM | 1321 | N | LEU | A | 701 | 4.904 | 10.426 | 5.135 | 1.00 | 25.36 |
| ATOM | 1322 | CA | LEU | A | 701 | 5.476 | 9.350 | 4.327 | 1.00 | 25.43 |
| ATOM | 1323 | CB | LEU | A | 701 | 4.460 | 8.218 | 4.146 | 1.00 | 25.64 |
| ATOM | 1324 | CG | LEU | A | 701 | 3.976 | 7.487 | 5.401 | 1.00 | 25.01 |
| ATOM | 1325 | CD1 | LEU | A | 701 | 2.975 | 6.403 | 5.008 | 1.00 | 25.21 |
| ATOM | 1326 | CD2 | LEU | A | 701 | 5.161 | 6.879 | 6.124 | 1.00 | 25.48 |
| ATOM | 1327 | C | LEU | A | 701 | 5.904 | 9.859 | 2.951 | 1.00 | 27.13 |
| ATOM | 1328 | O | LEU | A | 701 | 6.280 | 9.075 | 2.082 | 1.00 | 26.39 |
| ATOM | 1329 | N | SER | A | 702 | 5.851 | 11.172 | 2.764 | 1.00 | 36.40 |
| ATOM | 1330 | CA | SER | A | 702 | 6.218 | 11.787 | 1.488 | 1.00 | 38.48 |
| ATOM | 1331 | CB | SER | A | 702 | 6.077 | 13.303 | 1.581 | 1.00 | 34.20 |
| ATOM | 1332 | OG | SER | A | 702 | 7.065 | 13.837 | 2.446 | 1.00 | 34.77 |
| ATOM | 1333 | C | SER | A | 702 | 7.639 | 11.459 | 1.036 | 1.00 | 38.77 |
| ATOM | 1334 | O | SER | A | 702 | 7.915 | 11.380 | −0.162 | 1.00 | 39.14 |
| ATOM | 1335 | N | GLY | A | 703 | 8.537 | 11.278 | 1.997 | 1.00 | 37.92 |
| ATOM | 1336 | CA | GLY | A | 703 | 9.920 | 10.984 | 1.673 | 1.00 | 37.06 |
| ATOM | 1337 | C | GLY | A | 703 | 10.242 | 9.556 | 1.271 | 1.00 | 36.77 |
| ATOM | 1338 | O | GLY | A | 703 | 11.329 | 9.293 | 0.760 | 1.00 | 37.21 |
| ATOM | 1339 | N | LEU | A | 704 | 9.316 | 8.628 | 1.493 | 1.00 | 28.47 |
| ATOM | 1340 | CA | LEU | A | 704 | 9.566 | 7.231 | 1.138 | 1.00 | 27.53 |
| ATOM | 1341 | CB | LEU | A | 704 | 8.446 | 6.321 | 1.655 | 1.00 | 29.58 |
| ATOM | 1342 | CG | LEU | A | 704 | 8.208 | 6.233 | 3.161 | 1.00 | 30.45 |
| ATOM | 1343 | CD1 | LEU | A | 704 | 7.255 | 5.088 | 3.465 | 1.00 | 29.64 |
| ATOM | 1344 | CD2 | LEU | A | 704 | 9.510 | 6.005 | 3.855 | 1.00 | 30.15 |
| ATOM | 1345 | C | LEU | A | 704 | 9.682 | 7.037 | −0.364 | 1.00 | 26.70 |
| ATOM | 1346 | O | LEU | A | 704 | 9.092 | 7.783 | −1.142 | 1.00 | 27.33 |
| ATOM | 1347 | N | SER | A | 705 | 10.450 | 6.028 | −0.762 | 1.00 | 29.04 |
| ATOM | 1348 | CA | SER | A | 705 | 10.606 | 5.704 | −2.171 | 1.00 | 28.79 |
| ATOM | 1349 | CB | SER | A | 705 | 11.734 | 4.690 | −2.369 | 1.00 | 23.35 |
| ATOM | 1350 | OG | SER | A | 705 | 11.393 | 3.441 | −1.802 | 1.00 | 24.99 |
| ATOM | 1351 | C | SER | A | 705 | 9.275 | 5.062 | −2.540 | 1.00 | 29.09 |
| ATOM | 1352 | O | SER | A | 705 | 8.493 | 4.712 | −1.654 | 1.00 | 29.81 |
| ATOM | 1353 | N | ILE | A | 706 | 9.019 | 4.894 | −3.831 | 1.00 | 27.02 |
| ATOM | 1354 | CA | ILE | A | 706 | 7.764 | 4.295 | −4.268 | 1.00 | 26.47 |
| ATOM | 1355 | CB | ILE | A | 706 | 7.700 | 4.211 | −5.809 | 1.00 | 35.41 |
| ATOM | 1356 | CG2 | ILE | A | 706 | 6.441 | 3.472 | −6.241 | 1.00 | 36.09 |
| ATOM | 1357 | CG1 | ILE | A | 706 | 7.728 | 5.622 | −6.402 | 1.00 | 36.23 |
| ATOM | 1358 | CD1 | ILE | A | 706 | 7.821 | 5.658 | −7.917 | 1.00 | 36.02 |
| ATOM | 1359 | C | ILE | A | 706 | 7.561 | 2.902 | −3.681 | 1.00 | 26.18 |
| ATOM | 1360 | O | ILE | A | 706 | 6.487 | 2.584 | −3.175 | 1.00 | 26.02 |
| ATOM | 1361 | N | GLU | A | 707 | 8.594 | 2.071 | −3.736 | 1.00 | 25.11 |
| ATOM | 1362 | CA | GLU | A | 707 | 8.484 | 0.720 | −3.206 | 1.00 | 25.30 |
| ATOM | 1363 | CB | GLU | A | 707 | 9.725 | −0.092 | −3.577 | 1.00 | 46.28 |
| ATOM | 1364 | CG | GLU | A | 707 | 9.775 | −0.446 | −5.051 | 1.00 | 47.07 |
| ATOM | 1365 | CD | GLU | A | 707 | 8.667 | −1.406 | −5.452 | 1.00 | 47.32 |
| ATOM | 1366 | OE1 | GLU | A | 707 | 8.296 | −1.422 | −6.645 | 1.00 | 47.31 |
| ATOM | 1367 | OE2 | GLU | A | 707 | 8.177 | −2.153 | −4.576 | 1.00 | 47.02 |
| ATOM | 1368 | C | GLU | A | 707 | 8.284 | 0.730 | −1.692 | 1.00 | 25.18 |
| ATOM | 1369 | O | GLU | A | 707 | 7.483 | −0.042 | −1.164 | 1.00 | 24.41 |
| ATOM | 1370 | N | GLU | A | 708 | 9.005 | 1.610 | −1.002 | 1.00 | 23.37 |
| ATOM | 1371 | CA | GLU | A | 708 | 8.887 | 1.708 | 0.448 | 1.00 | 24.60 |
| ATOM | 1372 | CB | GLU | A | 708 | 9.904 | 2.704 | 1.011 | 1.00 | 30.84 |
| ATOM | 1373 | CG | GLU | A | 708 | 11.278 | 2.100 | 1.265 | 1.00 | 31.16 |
| ATOM | 1374 | CD | GLU | A | 708 | 12.236 | 3.077 | 1.921 | 1.00 | 31.10 |
| ATOM | 1375 | OE1 | GLU | A | 708 | 13.164 | 2.615 | 2.617 | 1.00 | 31.36 |
| ATOM | 1376 | OE2 | GLU | A | 708 | 12.073 | 4.301 | 1.732 | 1.00 | 31.87 |
| ATOM | 1377 | C | GLU | A | 708 | 7.481 | 2.138 | 0.839 | 1.00 | 24.78 |
| ATOM | 1378 | O | GLU | A | 708 | 6.886 | 1.579 | 1.768 | 1.00 | 23.94 |
| ATOM | 1379 | N | TYR | A | 709 | 6.961 | 3.133 | 0.128 | 1.00 | 23.21 |
| ATOM | 1380 | CA | TYR | A | 709 | 5.619 | 3.644 | 0.390 | 1.00 | 25.29 |
| ATOM | 1381 | CB | TYR | A | 709 | 5.295 | 4.802 | −0.550 | 1.00 | 27.48 |
| ATOM | 1382 | CG | TYR | A | 709 | 3.912 | 5.365 | −0.325 | 1.00 | 28.20 |
| ATOM | 1383 | CD1 | TYR | A | 709 | 3.654 | 6.221 | 0.743 | 1.00 | 28.35 |
| ATOM | 1384 | CE1 | TYR | A | 709 | 2.375 | 6.712 | 0.980 | 1.00 | 28.11 |
| ATOM | 1385 | CD2 | TYR | A | 709 | 2.853 | 5.011 | −1.157 | 1.00 | 29.10 |
| ATOM | 1386 | CE2 | TYR | A | 709 | 1.567 | 5.496 | −0.927 | 1.00 | 29.20 |
| ATOM | 1387 | CZ | TYR | A | 709 | 1.337 | 6.343 | 0.142 | 1.00 | 29.09 |
| ATOM | 1388 | OH | TYR | A | 709 | 0.065 | 6.813 | 0.379 | 1.00 | 28.65 |
| ATOM | 1389 | C | TYR | A | 709 | 4.568 | 2.549 | 0.224 | 1.00 | 25.53 |
| ATOM | 1390 | O | TYR | A | 709 | 3.740 | 2.336 | 1.111 | 1.00 | 24.94 |
| ATOM | 1391 | N | LYS | A | 710 | 4.602 | 1.859 | −0.915 | 1.00 | 25.86 |
| ATOM | 1392 | CA | LYS | A | 710 | 3.658 | 0.778 | −1.191 | 1.00 | 26.20 |
| ATOM | 1393 | CB | LYS | A | 710 | 3.955 | 0.144 | −2.551 | 1.00 | 48.58 |
| ATOM | 1394 | CG | LYS | A | 710 | 3.417 | −1.276 | −2.683 | 1.00 | 49.43 |
| ATOM | 1395 | CD | LYS | A | 710 | 3.795 | −1.914 | −4.008 | 1.00 | 49.56 |
| ATOM | 1396 | CE | LYS | A | 710 | 3.501 | −3.408 | −3.993 | 1.00 | 49.72 |
| ATOM | 1397 | NZ | LYS | A | 710 | 2.086 | −3.707 | −3.633 | 1.00 | 49.51 |
| ATOM | 1398 | C | LYS | A | 710 | 3.689 | −0.309 | −0.123 | 1.00 | 25.97 |
| ATOM | 1399 | O | LYS | A | 710 | 2.640 | −0.743 | 0.356 | 1.00 | 25.66 |
| ATOM | 1400 | N | THR | A | 711 | 4.891 | −0.756 | 0.236 | 1.00 | 24.32 |
| ATOM | 1401 | CA | THR | A | 711 | 5.049 | −1.800 | 1.240 | 1.00 | 23.82 |
| ATOM | 1402 | CB | THR | A | 711 | 6.528 | −2.205 | 1.399 | 1.00 | 33.44 |
| ATOM | 1403 | OG1 | THR | A | 711 | 7.030 | −2.673 | 0.141 | 1.00 | 33.80 |
| ATOM | 1404 | CG2 | THR | A | 711 | 6.669 | −3.307 | 2.441 | 1.00 | 33.11 |
| ATOM | 1405 | C | THR | A | 711 | 4.523 | −1.346 | 2.596 | 1.00 | 23.74 |
| ATOM | 1406 | O | THR | A | 711 | 3.861 | −2.106 | 3.295 | 1.00 | 24.01 |
| ATOM | 1407 | N | THR | A | 712 | 4.813 | −0.100 | 2.951 | 1.00 | 22.39 |
| ATOM | 1408 | CA | THR | A | 712 | 4.379 | 0.449 | 4.227 | 1.00 | 21.87 |
| ATOM | 1409 | CB | THR | A | 712 | 5.003 | 1.831 | 4.468 | 1.00 | 23.06 |
| ATOM | 1410 | OG1 | THR | A | 712 | 6.432 | 1.713 | 4.416 | 1.00 | 24.85 |
| ATOM | 1411 | CG2 | THR | A | 712 | 4.604 | 2.371 | 5.832 | 1.00 | 23.79 |
| ATOM | 1412 | C | THR | A | 712 | 2.859 | 0.548 | 4.330 | 1.00 | 21.27 |
| ATOM | 1413 | O | THR | A | 712 | 2.287 | 0.150 | 5.340 | 1.00 | 22.87 |
| ATOM | 1414 | N | LEU | A | 713 | 2.207 | 1.060 | 3.291 | 1.00 | 23.23 |
| ATOM | 1415 | CA | LEU | A | 713 | 0.751 | 1.189 | 3.314 | 1.00 | 22.85 |
| ATOM | 1416 | CB | LEU | A | 713 | 0.249 | 1.892 | 2.049 | 1.00 | 31.38 |
| ATOM | 1417 | CG | LEU | A | 713 | −0.070 | 3.382 | 2.200 | 1.00 | 32.26 |
| ATOM | 1418 | CD1 | LEU | A | 713 | −1.303 | 3.560 | 3.082 | 1.00 | 32.03 |
| ATOM | 1419 | CD2 | LEU | A | 713 | 1.117 | 4.106 | 2.804 | 1.00 | 32.84 |
| ATOM | 1420 | C | LEU | A | 713 | 0.051 | −0.154 | 3.473 | 1.00 | 22.64 |
| ATOM | 1421 | O | LEU | A | 713 | −0.966 | −0.251 | 4.159 | 1.00 | 22.10 |
| ATOM | 1422 | N | LYS | A | 714 | 0.605 | −1.187 | 2.844 | 1.00 | 20.65 |
| ATOM | 1423 | CA | LYS | A | 714 | 0.054 | −2.534 | 2.919 | 1.00 | 20.74 |
| ATOM | 1424 | CB | LYS | A | 714 | 0.822 | −3.457 | 1.970 | 1.00 | 43.81 |
| ATOM | 1425 | CG | LYS | A | 714 | 0.599 | −4.943 | 2.199 | 1.00 | 44.63 |
| ATOM | 1426 | CD | LYS | A | 714 | 1.492 | −5.772 | 1.278 | 1.00 | 44.93 |
| ATOM | 1427 | CE | LYS | A | 714 | 1.524 | −7.235 | 1.692 | 1.00 | 44.98 |
| ATOM | 1428 | NZ | LYS | A | 714 | 0.178 | −7.868 | 1.647 | 1.00 | 44.80 |
| ATOM | 1429 | C | LYS | A | 714 | 0.165 | −3.051 | 4.348 | 1.00 | 19.68 |
| ATOM | 1430 | O | LYS | A | 714 | −0.755 | −3.693 | 4.865 | 1.00 | 20.74 |
| ATOM | 1431 | N | ILE | A | 715 | 1.300 | −2.777 | 4.984 | 1.00 | 20.42 |
| ATOM | 1432 | CA | ILE | A | 715 | 1.504 | −3.216 | 6.348 | 1.00 | 20.28 |
| ATOM | 1433 | CB | ILE | A | 715 | 2.950 | −2.960 | 6.818 | 1.00 | 24.09 |
| ATOM | 1434 | CG2 | ILE | A | 715 | 3.105 | −3.399 | 8.264 | 1.00 | 24.82 |
| ATOM | 1435 | CG1 | ILE | A | 715 | 3.930 | −3.718 | 5.917 | 1.00 | 25.35 |
| ATOM | 1436 | CD1 | ILE | A | 715 | 5.381 | −3.440 | 6.231 | 1.00 | 24.56 |
| ATOM | 1437 | C | ILE | A | 715 | 0.539 | −2.476 | 7.257 | 1.00 | 19.19 |
| ATOM | 1438 | O | ILE | A | 715 | −0.035 | −3.073 | 8.161 | 1.00 | 19.49 |
| ATOM | 1439 | N | ILE | A | 716 | 0.367 | −1.180 | 7.005 | 1.00 | 19.27 |
| ATOM | 1440 | CA | ILE | A | 716 | −0.546 | −0.361 | 7.800 | 1.00 | 18.10 |
| ATOM | 1441 | CB | ILE | A | 716 | −0.519 | 1.112 | 7.345 | 1.00 | 20.08 |
| ATOM | 1442 | CG2 | ILE | A | 716 | −1.672 | 1.877 | 7.984 | 1.00 | 20.73 |
| ATOM | 1443 | CG1 | ILE | A | 716 | 0.828 | 1.743 | 7.717 | 1.00 | 19.90 |
| ATOM | 1444 | CD1 | ILE | A | 716 | 1.027 | 3.151 | 7.204 | 1.00 | 20.14 |
| ATOM | 1445 | C | ILE | A | 716 | −1.977 | −0.890 | 7.705 | 1.00 | 16.92 |
| ATOM | 1446 | O | ILE | A | 716 | −2.647 | −1.066 | 8.727 | 1.00 | 17.86 |
| ATOM | 1447 | N | LYS | A | 717 | −2.449 | −1.143 | 6.488 | 1.00 | 18.04 |
| ATOM | 1448 | CA | LYS | A | 717 | −3.808 | −1.656 | 6.315 | 1.00 | 19.57 |
| ATOM | 1449 | CB | LYS | A | 717 | −4.150 | −1.839 | 4.834 | 1.00 | 22.85 |
| ATOM | 1450 | CG | LYS | A | 717 | −5.590 | −2.297 | 4.619 | 1.00 | 22.42 |
| ATOM | 1451 | CD | LYS | A | 717 | −5.930 | −2.486 | 3.144 | 1.00 | 23.58 |
| ATOM | 1452 | CE | LYS | A | 717 | −7.357 | −2.986 | 2.980 | 1.00 | 22.98 |
| ATOM | 1453 | NZ | LYS | A | 717 | −7.730 | −3.216 | 1.552 | 1.00 | 24.81 |
| ATOM | 1454 | C | LYS | A | 717 | −3.977 | −2.985 | 7.042 | 1.00 | 19.43 |
| ATOM | 1455 | O | LYS | A | 717 | −4.957 | −3.197 | 7.753 | 1.00 | 18.92 |
| ATOM | 1456 | N | GLN | A | 718 | −3.024 | −3.894 | 6.870 | 1.00 | 19.29 |
| ATOM | 1457 | CA | GLN | A | 718 | −3.136 | −5.173 | 7.552 | 1.00 | 17.73 |
| ATOM | 1458 | CB | GLN | A | 718 | −2.009 | −6.099 | 7.096 | 1.00 | 35.14 |
| ATOM | 1459 | CG | GLN | A | 718 | −2.134 | −6.456 | 5.621 | 1.00 | 35.99 |
| ATOM | 1460 | CD | GLN | A | 718 | −0.956 | −7.249 | 5.095 | 1.00 | 36.88 |
| ATOM | 1461 | OE1 | GLN | A | 718 | −0.973 | −7.718 | 3.956 | 1.00 | 37.13 |
| ATOM | 1462 | NE2 | GLN | A | 718 | 0.076 | −7.396 | 5.915 | 1.00 | 36.21 |
| ATOM | 1463 | C | GLN | A | 718 | −3.144 | −5.031 | 9.073 | 1.00 | 16.21 |
| ATOM | 1464 | O | GLN | A | 718 | −3.899 | −5.725 | 9.764 | 1.00 | 16.17 |
| ATOM | 1465 | N | ALA | A | 719 | −2.326 | −4.117 | 9.585 | 1.00 | 17.17 |
| ATOM | 1466 | CA | ALA | A | 719 | −2.236 | −3.886 | 11.020 | 1.00 | 16.40 |
| ATOM | 1467 | CB | ALA | A | 719 | −1.062 | −2.939 | 11.335 | 1.00 | 18.08 |
| ATOM | 1468 | C | ALA | A | 719 | −3.542 | −3.325 | 11.577 | 1.00 | 15.71 |
| ATOM | 1469 | O | ALA | A | 719 | −3.996 | −3.754 | 12.637 | 1.00 | 15.47 |
| ATOM | 1470 | N | ILE | A | 720 | −4.150 | −2.380 | 10.864 | 1.00 | 15.28 |
| ATOM | 1471 | CA | ILE | A | 720 | −5.402 | −1.802 | 11.330 | 1.00 | 17.34 |
| ATOM | 1472 | CB | ILE | A | 720 | −5.758 | −0.520 | 10.525 | 1.00 | 16.58 |
| ATOM | 1473 | CG2 | ILE | A | 720 | −7.185 | −0.054 | 10.855 | 1.00 | 17.16 |
| ATOM | 1474 | CG1 | ILE | A | 720 | −4.736 | 0.570 | 10.852 | 1.00 | 17.26 |
| ATOM | 1475 | CD1 | ILE | A | 720 | −5.042 | 1.931 | 10.241 | 1.00 | 17.39 |
| ATOM | 1476 | C | ILE | A | 720 | −6.516 | −2.847 | 11.257 | 1.00 | 14.75 |
| ATOM | 1477 | O | ILE | A | 720 | −7.288 | −3.002 | 12.199 | 1.00 | 16.06 |
| ATOM | 1478 | N | LEU | A | 721 | −6.583 | −3.605 | 10.165 | 1.00 | 16.72 |
| ATOM | 1479 | CA | LEU | A | 721 | −7.617 | −4.629 | 10.070 | 1.00 | 17.03 |
| ATOM | 1480 | CB | LEU | A | 721 | −7.616 | −5.282 | 8.684 | 1.00 | 21.51 |
| ATOM | 1481 | CG | LEU | A | 721 | −8.136 | −4.398 | 7.544 | 1.00 | 22.77 |
| ATOM | 1482 | CD1 | LEU | A | 721 | −8.089 | −5.164 | 6.236 | 1.00 | 22.53 |
| ATOM | 1483 | CD2 | LEU | A | 721 | −9.556 | −3.947 | 7.834 | 1.00 | 22.98 |
| ATOM | 1484 | C | LEU | A | 721 | −7.442 | −5.694 | 11.148 | 1.00 | 15.19 |
| ATOM | 1485 | O | LEU | A | 721 | −8.416 | −6.269 | 11.624 | 1.00 | 16.45 |
| ATOM | 1486 | N | ALA | A | 722 | −6.199 | −5.940 | 11.546 | 1.00 | 17.48 |
| ATOM | 1487 | CA | ALA | A | 722 | −5.924 | −6.935 | 12.571 | 1.00 | 17.80 |
| ATOM | 1488 | CB | ALA | A | 722 | −4.431 | −7.113 | 12.735 | 1.00 | 18.14 |
| ATOM | 1489 | C | ALA | A | 722 | −6.547 | −6.557 | 13.905 | 1.00 | 18.00 |
| ATOM | 1490 | O | ALA | A | 722 | −6.764 | −7.420 | 14.750 | 1.00 | 18.41 |
| ATOM | 1491 | N | THR | A | 723 | −6.854 | −5.274 | 14.094 | 1.00 | 15.61 |
| ATOM | 1492 | CA | THR | A | 723 | −7.434 | −4.856 | 15.359 | 1.00 | 15.90 |
| ATOM | 1493 | CB | THR | A | 723 | −7.199 | −3.360 | 15.650 | 1.00 | 16.83 |
| ATOM | 1494 | OG1 | THR | A | 723 | −7.801 | −2.550 | 14.635 | 1.00 | 17.34 |
| ATOM | 1495 | CG2 | THR | A | 723 | −5.701 | −3.084 | 15.726 | 1.00 | 18.33 |
| ATOM | 1496 | C | THR | A | 723 | −8.909 | −5.192 | 15.499 | 1.00 | 16.50 |
| ATOM | 1497 | O | THR | A | 723 | −9.542 | −4.838 | 16.492 | 1.00 | 17.29 |
| ATOM | 1498 | N | ASP | A | 724 | −9.452 | −5.875 | 14.498 | 1.00 | 17.56 |
| ATOM | 1499 | CA | ASP | A | 724 | −10.826 | −6.342 | 14.549 | 1.00 | 18.40 |
| ATOM | 1500 | CB | ASP | A | 724 | −11.304 | −6.762 | 13.157 | 1.00 | 16.11 |
| ATOM | 1501 | CG | ASP | A | 724 | −12.737 | −7.273 | 13.160 | 1.00 | 14.99 |
| ATOM | 1502 | OD1 | ASP | A | 724 | −13.297 | −7.492 | 14.254 | 1.00 | 16.02 |
| ATOM | 1503 | OD2 | ASP | A | 724 | −13.310 | −7.458 | 12.069 | 1.00 | 18.73 |
| ATOM | 1504 | C | ASP | A | 724 | −10.707 | −7.582 | 15.431 | 1.00 | 18.02 |
| ATOM | 1505 | O | ASP | A | 724 | −10.114 | −8.571 | 15.006 | 1.00 | 18.53 |
| ATOM | 1506 | N | LEU | A | 725 | −11.242 | −7.536 | 16.649 | 1.00 | 20.52 |
| ATOM | 1507 | CA | LEU | A | 725 | −11.140 | −8.685 | 17.545 | 1.00 | 21.37 |
| ATOM | 1508 | CB | LEU | A | 725 | −11.851 | −8.407 | 18.873 | 1.00 | 31.10 |
| ATOM | 1509 | CG | LEU | A | 725 | −10.960 | −7.911 | 20.016 | 1.00 | 32.17 |
| ATOM | 1510 | CD1 | LEU | A | 725 | −11.782 | −7.798 | 21.293 | 1.00 | 32.32 |
| ATOM | 1511 | CD2 | LEU | A | 725 | −9.809 | −8.885 | 20.232 | 1.00 | 31.61 |
| ATOM | 1512 | C | LEU | A | 725 | −11.673 | −9.981 | 16.942 | 1.00 | 22.61 |
| ATOM | 1513 | O | LEU | A | 725 | −11.229 | −11.061 | 17.316 | 1.00 | 22.86 |
| ATOM | 1514 | N | ALA | A | 726 | −12.613 | −9.887 | 16.006 | 1.00 | 24.20 |
| ATOM | 1515 | CA | ALA | A | 726 | −13.158 | −11.095 | 15.376 | 1.00 | 26.25 |
| ATOM | 1516 | CB | ALA | A | 726 | −14.375 | −10.746 | 14.520 | 1.00 | 26.36 |
| ATOM | 1517 | C | ALA | A | 726 | −12.093 | −11.782 | 14.520 | 1.00 | 25.26 |
| ATOM | 1518 | O | ALA | A | 726 | −12.043 | −13.015 | 14.431 | 1.00 | 26.23 |
| ATOM | 1519 | N | LEU | A | 727 | −11.253 | −10.981 | 13.879 | 1.00 | 25.84 |
| ATOM | 1520 | CA | LEU | A | 727 | −10.183 | −11.497 | 13.037 | 1.00 | 27.01 |
| ATOM | 1521 | CB | LEU | A | 727 | −9.582 | −10.354 | 12.217 | 1.00 | 27.22 |
| ATOM | 1522 | CG | LEU | A | 727 | −9.355 | −10.566 | 10.718 | 1.00 | 29.26 |
| ATOM | 1523 | CD1 | LEU | A | 727 | −10.607 | −11.167 | 10.073 | 1.00 | 27.54 |
| ATOM | 1524 | CD2 | LEU | A | 727 | −9.011 | −9.229 | 10.069 | 1.00 | 28.24 |
| ATOM | 1525 | C | LEU | A | 727 | −9.123 | −12.118 | 13.946 | 1.00 | 26.83 |
| ATOM | 1526 | O | LEU | A | 727 | −8.441 | −13.077 | 13.575 | 1.00 | 27.75 |
| ATOM | 1527 | N | TYR | A | 728 | −8.982 | −11.566 | 15.144 | 1.00 | 23.72 |
| ATOM | 1528 | CA | TYR | A | 728 | −8.013 | −12.096 | 16.094 | 1.00 | 24.54 |
| ATOM | 1529 | CB | TYR | A | 728 | −7.843 | −11.144 | 17.280 | 1.00 | 26.68 |
| ATOM | 1530 | CG | TYR | A | 728 | −7.128 | −11.784 | 18.440 | 1.00 | 27.14 |
| ATOM | 1531 | CD1 | TYR | A | 728 | −7.828 | −12.208 | 19.568 | 1.00 | 27.32 |
| ATOM | 1532 | CE1 | TYR | A | 728 | −7.183 | −12.878 | 20.602 | 1.00 | 27.41 |
| ATOM | 1533 | CD2 | TYR | A | 728 | −5.762 | −12.040 | 18.377 | 1.00 | 27.43 |
| ATOM | 1534 | CE2 | TYR | A | 728 | −5.111 | −12.709 | 19.400 | 1.00 | 27.27 |
| ATOM | 1535 | CZ | TYR | A | 728 | −5.825 | −13.126 | 20.505 | 1.00 | 27.40 |
| ATOM | 1536 | OH | TYR | A | 728 | −5.178 | −13.810 | 21.507 | 1.00 | 28.59 |
| ATOM | 1537 | C | TYR | A | 728 | −8.483 | −13.462 | 16.593 | 1.00 | 24.46 |
| ATOM | 1538 | O | TYR | A | 728 | −7.713 | −14.425 | 16.643 | 1.00 | 24.91 |
| ATOM | 1539 | N | ILE | A | 729 | −9.753 | −13.536 | 16.968 | 1.00 | 25.84 |
| ATOM | 1540 | CA | ILE | A | 729 | −10.335 | −14.779 | 17.463 | 1.00 | 26.31 |
| ATOM | 1541 | CB | ILE | A | 729 | −11.803 | −14.555 | 17.876 | 1.00 | 28.61 |
| ATOM | 1542 | CG2 | ILE | A | 729 | −12.482 | −15.887 | 18.155 | 1.00 | 28.30 |
| ATOM | 1543 | CG1 | ILE | A | 729 | −11.855 | −13.646 | 19.106 | 1.00 | 27.91 |
| ATOM | 1544 | CD1 | ILE | A | 729 | −13.251 | −13.165 | 19.462 | 1.00 | 28.85 |
| ATOM | 1545 | C | ILE | A | 729 | −10.265 | −15.874 | 16.401 | 1.00 | 28.18 |
| ATOM | 1546 | O | ILE | A | 729 | −10.162 | −17.061 | 16.727 | 1.00 | 28.43 |
| ATOM | 1547 | N | LYS | A | 730 | −10.304 | −15.469 | 15.135 | 1.00 | 32.05 |
| ATOM | 1548 | CA | LYS | A | 730 | −10.265 | −16.402 | 14.008 | 1.00 | 33.23 |
| ATOM | 1549 | CB | LYS | A | 730 | −10.833 | −15.714 | 12.758 | 1.00 | 40.71 |
| ATOM | 1550 | CG | LYS | A | 730 | −10.709 | −16.526 | 11.475 | 1.00 | 40.87 |
| ATOM | 1551 | CD | LYS | A | 730 | −11.146 | −15.727 | 10.251 | 1.00 | 40.83 |
| ATOM | 1552 | CE | LYS | A | 730 | −11.013 | −16.555 | 8.977 | 1.00 | 41.35 |
| ATOM | 1553 | NZ | LYS | A | 730 | −11.478 | −15.810 | 7.772 | 1.00 | 41.21 |
| ATOM | 1554 | C | LYS | A | 730 | −8.865 | −16.938 | 13.697 | 1.00 | 33.75 |
| ATOM | 1555 | O | LYS | A | 730 | −8.688 | −18.137 | 13.457 | 1.00 | 33.39 |
| ATOM | 1556 | N | ARG | A | 731 | −7.876 | −16.050 | 13.703 | 1.00 | 31.85 |
| ATOM | 1557 | CA | ARG | A | 731 | −6.503 | −16.428 | 13.380 | 1.00 | 31.83 |
| ATOM | 1558 | CB | ARG | A | 731 | −5.825 | −15.293 | 12.604 | 1.00 | 43.64 |
| ATOM | 1559 | CG | ARG | A | 731 | −6.375 | −15.070 | 11.205 | 1.00 | 44.14 |
| ATOM | 1560 | CD | ARG | A | 731 | −5.975 | −13.707 | 10.644 | 1.00 | 44.16 |
| ATOM | 1561 | NE | ARG | A | 731 | −4.534 | −13.536 | 10.456 | 1.00 | 44.52 |
| ATOM | 1562 | CZ | ARG | A | 731 | −3.809 | −14.173 | 9.541 | 1.00 | 44.27 |
| ATOM | 1563 | NH1 | ARG | A | 731 | −4.383 | −15.039 | 8.716 | 1.00 | 44.55 |
| ATOM | 1564 | NH2 | ARG | A | 731 | −2.507 | −13.933 | 9.440 | 1.00 | 44.01 |
| ATOM | 1565 | C | ARG | A | 731 | −5.603 | −16.813 | 14.549 | 1.00 | 31.39 |
| ATOM | 1566 | O | ARG | A | 731 | −4.510 | −17.339 | 14.333 | 1.00 | 31.88 |
| ATOM | 1567 | N | ARG | A | 732 | −6.040 | −16.566 | 15.780 | 1.00 | 27.33 |
| ATOM | 1568 | CA | ARG | A | 732 | −5.189 | −16.880 | 16.923 | 1.00 | 27.09 |
| ATOM | 1569 | CB | ARG | A | 732 | −5.783 | −16.315 | 18.218 | 1.00 | 27.95 |
| ATOM | 1570 | CG | ARG | A | 732 | −7.063 | −16.972 | 18.680 | 1.00 | 27.63 |
| ATOM | 1571 | CD | ARG | A | 732 | −7.423 | −16.469 | 20.056 | 1.00 | 27.47 |
| ATOM | 1572 | NE | ARG | A | 732 | −8.557 | −17.178 | 20.632 | 1.00 | 27.81 |
| ATOM | 1573 | CZ | ARG | A | 732 | −8.915 | −17.083 | 21.907 | 1.00 | 28.15 |
| ATOM | 1574 | NH1 | ARG | A | 732 | −8.225 | −16.308 | 22.735 | 1.00 | 28.26 |
| ATOM | 1575 | NH2 | ARG | A | 732 | −9.955 | −17.772 | 22.358 | 1.00 | 29.20 |
| ATOM | 1576 | C | ARG | A | 732 | −4.928 | −18.372 | 17.086 | 1.00 | 26.60 |
| ATOM | 1577 | O | ARG | A | 732 | −3.853 | −18.767 | 17.524 | 1.00 | 26.91 |
| ATOM | 1578 | N | GLY | A | 733 | −5.909 | −19.195 | 16.733 | 1.00 | 28.21 |
| ATOM | 1579 | CA | GLY | A | 733 | −5.741 | −20.633 | 16.860 | 1.00 | 28.94 |
| ATOM | 1580 | C | GLY | A | 733 | −4.533 | −21.138 | 16.097 | 1.00 | 28.70 |
| ATOM | 1581 | O | GLY | A | 733 | −3.785 | −21.991 | 16.582 | 1.00 | 29.01 |
| ATOM | 1582 | N | GLU | A | 734 | −4.344 | −20.609 | 14.895 | 1.00 | 26.13 |
| ATOM | 1583 | CA | GLU | A | 734 | −3.224 | −20.999 | 14.049 | 1.00 | 26.64 |
| ATOM | 1584 | CB | GLU | A | 734 | −3.257 | −20.213 | 12.741 | 1.00 | 32.54 |
| ATOM | 1585 | CG | GLU | A | 734 | −2.289 | −20.720 | 11.697 | 1.00 | 32.73 |
| ATOM | 1586 | CD | GLU | A | 734 | −2.261 | −19.855 | 10.458 | 1.00 | 32.93 |
| ATOM | 1587 | OE1 | GLU | A | 734 | −3.330 | −19.331 | 10.068 | 1.00 | 32.62 |
| ATOM | 1588 | OE2 | GLU | A | 734 | −1.168 | −19.713 | 9.867 | 1.00 | 33.12 |
| ATOM | 1589 | C | GLU | A | 734 | −1.927 | −20.705 | 14.783 | 1.00 | 26.89 |
| ATOM | 1590 | O | GLU | A | 734 | −1.071 | −21.571 | 14.935 | 1.00 | 26.89 |
| ATOM | 1591 | N | PHE | A | 735 | −1.797 | −19.463 | 15.233 | 1.00 | 26.37 |
| ATOM | 1592 | CA | PHE | A | 735 | −0.630 | −18.996 | 15.965 | 1.00 | 26.70 |
| ATOM | 1593 | CB | PHE | A | 735 | −0.911 | −17.569 | 16.455 | 1.00 | 35.10 |
| ATOM | 1594 | CG | PHE | A | 735 | 0.263 | −16.884 | 17.099 | 1.00 | 35.60 |
| ATOM | 1595 | CD1 | PHE | A | 735 | 1.487 | −16.801 | 16.448 | 1.00 | 35.44 |
| ATOM | 1596 | CD2 | PHE | A | 735 | 0.116 | −16.256 | 18.333 | 1.00 | 35.57 |
| ATOM | 1597 | CE1 | PHE | A | 735 | 2.549 | −16.098 | 17.013 | 1.00 | 35.65 |
| ATOM | 1598 | CE2 | PHE | A | 735 | 1.168 | −15.550 | 18.909 | 1.00 | 35.48 |
| ATOM | 1599 | CZ | PHE | A | 735 | 2.388 | −15.469 | 18.247 | 1.00 | 35.90 |
| ATOM | 1600 | C | PHE | A | 735 | −0.309 | −19.919 | 17.148 | 1.00 | 26.06 |
| ATOM | 1601 | O | PHE | A | 735 | 0.819 | −20.387 | 17.297 | 1.00 | 26.34 |
| ATOM | 1602 | N | PHE | A | 736 | −1.313 | −20.186 | 17.978 | 1.00 | 27.83 |
| ATOM | 1603 | CA | PHE | A | 736 | −1.144 | −21.049 | 19.146 | 1.00 | 27.77 |
| ATOM | 1604 | CB | PHE | A | 736 | −2.462 | −21.134 | 19.925 | 1.00 | 26.06 |
| ATOM | 1605 | CG | PHE | A | 736 | −2.927 | −19.810 | 20.492 | 1.00 | 26.21 |
| ATOM | 1606 | CD1 | PHE | A | 736 | −4.239 | −19.659 | 20.948 | 1.00 | 25.79 |
| ATOM | 1607 | CD2 | PHE | A | 736 | −2.059 | −18.727 | 20.587 | 1.00 | 25.60 |
| ATOM | 1608 | CE1 | PHE | A | 736 | −4.676 | −18.450 | 21.489 | 1.00 | 25.76 |
| ATOM | 1609 | CE2 | PHE | A | 736 | −2.490 | −17.508 | 21.130 | 1.00 | 25.65 |
| ATOM | 1610 | CZ | PHE | A | 736 | −3.801 | −17.376 | 21.580 | 1.00 | 25.15 |
| ATOM | 1611 | C | PHE | A | 736 | −0.692 | −22.453 | 18.737 | 1.00 | 27.64 |
| ATOM | 1612 | O | PHE | A | 736 | 0.212 | −23.020 | 19.352 | 1.00 | 28.68 |
| ATOM | 1613 | N | GLU | A | 737 | −1.335 | −23.001 | 17.709 | 1.00 | 26.38 |
| ATOM | 1614 | CA | GLU | A | 737 | −1.015 | −24.335 | 17.187 | 1.00 | 26.87 |
| ATOM | 1615 | CB | GLU | A | 737 | −1.860 | −24.633 | 15.943 | 1.00 | 54.89 |
| ATOM | 1616 | CG | GLU | A | 737 | −3.324 | −24.925 | 16.215 | 1.00 | 55.77 |
| ATOM | 1617 | CD | GLU | A | 737 | −3.541 | −26.319 | 16.765 | 1.00 | 56.24 |
| ATOM | 1618 | OE1 | GLU | A | 737 | −3.171 | −27.293 | 16.074 | 1.00 | 56.16 |
| ATOM | 1619 | OE2 | GLU | A | 737 | −4.081 | −26.440 | 17.885 | 1.00 | 55.95 |
| ATOM | 1620 | C | GLU | A | 737 | 0.458 | −24.431 | 16.809 | 1.00 | 26.60 |
| ATOM | 1621 | O | GLU | A | 737 | 1.158 | −25.385 | 17.181 | 1.00 | 24.87 |
| ATOM | 1622 | N | LEU | A | 738 | 0.919 | −23.446 | 16.047 | 1.00 | 24.73 |
| ATOM | 1623 | CA | LEU | A | 738 | 2.304 | −23.402 | 15.603 | 1.00 | 25.81 |
| ATOM | 1624 | CB | LEU | A | 738 | 2.558 | −22.131 | 14.790 | 1.00 | 23.18 |
| ATOM | 1625 | CG | LEU | A | 738 | 2.098 | −22.127 | 13.326 | 1.00 | 24.63 |
| ATOM | 1626 | CD1 | LEU | A | 738 | 0.728 | −22.761 | 13.172 | 1.00 | 25.88 |
| ATOM | 1627 | CD2 | LEU | A | 738 | 2.093 | −20.699 | 12.816 | 1.00 | 24.42 |
| ATOM | 1628 | C | LEU | A | 738 | 3.271 | −23.459 | 16.771 | 1.00 | 25.22 |
| ATOM | 1629 | O | LEU | A | 738 | 4.259 | −24.190 | 16.730 | 1.00 | 26.20 |
| ATOM | 1630 | N | ILE | A | 739 | 2.992 | −22.676 | 17.809 | 1.00 | 22.89 |
| ATOM | 1631 | CA | ILE | A | 739 | 3.851 | −22.643 | 18.983 | 1.00 | 23.09 |
| ATOM | 1632 | CB | ILE | A | 739 | 3.406 | −21.531 | 19.961 | 1.00 | 29.33 |
| ATOM | 1633 | CG2 | ILE | A | 739 | 4.227 | −21.594 | 21.237 | 1.00 | 29.45 |
| ATOM | 1634 | CG1 | ILE | A | 739 | 3.554 | −20.161 | 19.293 | 1.00 | 29.28 |
| ATOM | 1635 | CD1 | ILE | A | 739 | 4.978 | −19.789 | 18.968 | 1.00 | 29.71 |
| ATOM | 1636 | C | ILE | A | 739 | 3.850 | −23.981 | 19.717 | 1.00 | 23.52 |
| ATOM | 1637 | O | ILE | A | 739 | 4.904 | −24.513 | 20.040 | 1.00 | 24.40 |
| ATOM | 1638 | N | ARG | A | 740 | 2.665 | −24.523 | 19.971 | 1.00 | 25.73 |
| ATOM | 1639 | CA | ARG | A | 740 | 2.545 | −25.790 | 20.683 | 1.00 | 27.35 |
| ATOM | 1640 | CB | ARG | A | 740 | 1.070 | −26.135 | 20.897 | 1.00 | 48.66 |
| ATOM | 1641 | CG | ARG | A | 740 | 0.840 | −27.418 | 21.679 | 1.00 | 49.28 |
| ATOM | 1642 | CD | ARG | A | 740 | −0.625 | −27.566 | 22.043 | 1.00 | 49.58 |
| ATOM | 1643 | NE | ARG | A | 740 | −1.484 | −27.521 | 20.863 | 1.00 | 49.67 |
| ATOM | 1644 | CZ | ARG | A | 740 | −1.504 | −28.454 | 19.916 | 1.00 | 49.81 |
| ATOM | 1645 | NH1 | ARG | A | 740 | −0.709 | −29.514 | 20.008 | 1.00 | 50.00 |
| ATOM | 1646 | NH2 | ARG | A | 740 | −2.317 | −28.330 | 18.876 | 1.00 | 49.79 |
| ATOM | 1647 | C | ARG | A | 740 | 3.241 | −26.950 | 19.975 | 1.00 | 27.41 |
| ATOM | 1648 | O | ARG | A | 740 | 3.784 | −27.845 | 20.628 | 1.00 | 27.24 |
| ATOM | 1649 | N | LYS | A | 741 | 3.230 | −26.935 | 18.646 | 1.00 | 27.03 |
| ATOM | 1650 | CA | LYS | A | 741 | 3.863 | −27.999 | 17.874 | 1.00 | 27.38 |
| ATOM | 1651 | CB | LYS | A | 741 | 3.055 | −28.279 | 16.605 | 1.00 | 33.72 |
| ATOM | 1652 | CG | LYS | A | 741 | 1.670 | −28.843 | 16.885 | 1.00 | 34.15 |
| ATOM | 1653 | CD | LYS | A | 741 | 0.952 | −29.259 | 15.614 | 1.00 | 34.24 |
| ATOM | 1654 | CE | LYS | A | 741 | −0.401 | −29.874 | 15.943 | 1.00 | 34.27 |
| ATOM | 1655 | NZ | LYS | A | 741 | −1.166 | −30.255 | 14.726 | 1.00 | 34.95 |
| ATOM | 1656 | C | LYS | A | 741 | 5.315 | −27.692 | 17.511 | 1.00 | 27.29 |
| ATOM | 1657 | O | LYS | A | 741 | 5.937 | −28.430 | 16.745 | 1.00 | 28.07 |
| ATOM | 1658 | N | ASN | A | 742 | 5.854 | −26.606 | 18.061 | 1.00 | 25.30 |
| ATOM | 1659 | CA | ASN | A | 742 | 7.239 | −26.224 | 17.797 | 1.00 | 25.09 |
| ATOM | 1660 | CB | ASN | A | 742 | 8.189 | −27.297 | 18.332 | 1.00 | 29.88 |
| ATOM | 1661 | CG | ASN | A | 742 | 8.202 | −27.351 | 19.837 | 1.00 | 30.15 |
| ATOM | 1662 | OD1 | ASN | A | 742 | 8.592 | −26.389 | 20.491 | 1.00 | 30.86 |
| ATOM | 1663 | ND2 | ASN | A | 742 | 7.768 | −28.474 | 20.400 | 1.00 | 30.30 |
| ATOM | 1664 | C | ASN | A | 742 | 7.498 | −26.005 | 16.318 | 1.00 | 24.75 |
| ATOM | 1665 | O | ASN | A | 742 | 8.526 | −26.431 | 15.782 | 1.00 | 23.22 |
| ATOM | 1666 | N | GLN | A | 743 | 6.564 | −25.319 | 15.665 | 1.00 | 27.53 |
| ATOM | 1667 | CA | GLN | A | 743 | 6.670 | −25.035 | 14.240 | 1.00 | 28.70 |
| ATOM | 1668 | CB | GLN | A | 743 | 5.451 | −25.592 | 13.495 | 1.00 | 37.55 |
| ATOM | 1669 | CG | GLN | A | 743 | 5.093 | −27.027 | 13.845 | 1.00 | 37.58 |
| ATOM | 1670 | CD | GLN | A | 743 | 3.960 | −27.566 | 12.992 | 1.00 | 38.11 |
| ATOM | 1671 | OE1 | GLN | A | 743 | 2.923 | −26.918 | 12.830 | 1.00 | 38.11 |
| ATOM | 1672 | NE2 | GLN | A | 743 | 4.147 | −28.763 | 12.447 | 1.00 | 37.75 |
| ATOM | 1673 | C | GLN | A | 743 | 6.755 | −23.533 | 13.979 | 1.00 | 28.82 |
| ATOM | 1674 | O | GLN | A | 743 | 6.881 | −23.105 | 12.832 | 1.00 | 28.92 |
| ATOM | 1675 | N | PHE | A | 744 | 6.681 | −22.729 | 15.035 | 1.00 | 28.91 |
| ATOM | 1676 | CA | PHE | A | 744 | 6.743 | −21.284 | 14.850 | 1.00 | 29.47 |
| ATOM | 1677 | CB | PHE | A | 744 | 6.432 | −20.556 | 16.157 | 1.00 | 40.72 |
| ATOM | 1678 | CG | PHE | A | 744 | 6.298 | −19.064 | 15.997 | 1.00 | 40.67 |
| ATOM | 1679 | CD1 | PHE | A | 744 | 5.279 | −18.525 | 15.214 | 1.00 | 41.29 |
| ATOM | 1680 | CD2 | PHE | A | 744 | 7.191 | −18.200 | 16.618 | 1.00 | 41.11 |
| ATOM | 1681 | CE1 | PHE | A | 744 | 5.153 | −17.145 | 15.053 | 1.00 | 41.25 |
| ATOM | 1682 | CE2 | PHE | A | 744 | 7.074 | −16.819 | 16.464 | 1.00 | 41.16 |
| ATOM | 1683 | CZ | PHE | A | 744 | 6.053 | −16.292 | 15.680 | 1.00 | 41.33 |
| ATOM | 1684 | C | PHE | A | 744 | 8.097 | −20.819 | 14.326 | 1.00 | 29.13 |
| ATOM | 1685 | O | PHE | A | 744 | 9.146 | −21.260 | 14.792 | 1.00 | 30.17 |
| ATOM | 1686 | N | ASN | A | 745 | 8.055 | −19.921 | 13.349 | 1.00 | 28.18 |
| ATOM | 1687 | CA | ASN | A | 745 | 9.260 | −19.363 | 12.750 | 1.00 | 29.83 |
| ATOM | 1688 | CB | ASN | A | 745 | 9.795 | −20.272 | 11.648 | 1.00 | 38.97 |
| ATOM | 1689 | CG | ASN | A | 745 | 10.919 | −19.622 | 10.867 | 1.00 | 39.20 |
| ATOM | 1690 | OD1 | ASN | A | 745 | 11.985 | −19.348 | 11.411 | 1.00 | 39.54 |
| ATOM | 1691 | ND2 | ASN | A | 745 | 10.679 | −19.357 | 9.590 | 1.00 | 40.15 |
| ATOM | 1692 | C | ASN | A | 745 | 8.941 | −18.005 | 12.149 | 1.00 | 29.56 |
| ATOM | 1693 | O | ASN | A | 745 | 8.208 | −17.914 | 11.165 | 1.00 | 29.59 |
| ATOM | 1694 | N | LEU | A | 746 | 9.499 | −16.956 | 12.741 | 1.00 | 38.07 |
| ATOM | 1695 | CA | LEU | A | 746 | 9.268 | −15.597 | 12.270 | 1.00 | 39.23 |
| ATOM | 1696 | CB | LEU | A | 746 | 9.822 | −14.591 | 13.280 | 1.00 | 38.00 |
| ATOM | 1697 | CG | LEU | A | 746 | 8.967 | −14.381 | 14.528 | 1.00 | 37.62 |
| ATOM | 1698 | CD1 | LEU | A | 746 | 9.676 | −13.427 | 15.475 | 1.00 | 38.20 |
| ATOM | 1699 | CD2 | LEU | A | 746 | 7.609 | −13.821 | 14.125 | 1.00 | 37.68 |
| ATOM | 1700 | C | LEU | A | 746 | 9.852 | −15.302 | 10.895 | 1.00 | 39.66 |
| ATOM | 1701 | O | LEU | A | 746 | 9.455 | −14.335 | 10.245 | 1.00 | 39.51 |
| ATOM | 1702 | N | GLU | A | 747 | 10.792 | −16.128 | 10.449 | 1.00 | 39.64 |
| ATOM | 1703 | CA | GLU | A | 747 | 11.404 | −15.918 | 9.143 | 1.00 | 39.71 |
| ATOM | 1704 | CB | GLU | A | 747 | 12.605 | −16.845 | 8.965 | 1.00 | 45.59 |
| ATOM | 1705 | CG | GLU | A | 747 | 13.921 | −16.094 | 8.881 | 1.00 | 46.56 |
| ATOM | 1706 | CD | GLU | A | 747 | 15.125 | −16.981 | 9.132 | 1.00 | 46.55 |
| ATOM | 1707 | OE1 | GLU | A | 747 | 15.305 | −17.974 | 8.395 | 1.00 | 46.54 |
| ATOM | 1708 | OE2 | GLU | A | 747 | 15.890 | −16.679 | 10.072 | 1.00 | 46.60 |
| ATOM | 1709 | C | GLU | A | 747 | 10.391 | −16.136 | 8.029 | 1.00 | 38.87 |
| ATOM | 1710 | O | GLU | A | 747 | 10.422 | −15.454 | 7.002 | 1.00 | 39.21 |
| ATOM | 1711 | N | ASP | A | 748 | 9.487 | −17.086 | 8.234 | 1.00 | 32.77 |
| ATOM | 1712 | CA | ASP | A | 748 | 8.455 | −17.363 | 7.248 | 1.00 | 32.31 |
| ATOM | 1713 | CB | ASP | A | 748 | 7.654 | −18.600 | 7.652 | 1.00 | 47.03 |
| ATOM | 1714 | CG | ASP | A | 748 | 6.639 | −19.002 | 6.603 | 1.00 | 47.67 |
| ATOM | 1715 | OD1 | ASP | A | 748 | 5.613 | −18.305 | 6.459 | 1.00 | 47.81 |
| ATOM | 1716 | OD2 | ASP | A | 748 | 6.876 | −20.018 | 5.912 | 1.00 | 48.36 |
| ATOM | 1717 | C | ASP | A | 748 | 7.544 | −16.141 | 7.190 | 1.00 | 32.61 |
| ATOM | 1718 | O | ASP | A | 748 | 6.915 | −15.777 | 8.185 | 1.00 | 30.88 |
| ATOM | 1719 | N | PRO | A | 749 | 7.466 | −15.490 | 6.018 | 1.00 | 39.12 |
| ATOM | 1720 | CD | PRO | A | 749 | 8.049 | −15.930 | 4.739 | 1.00 | 38.16 |
| ATOM | 1721 | CA | PRO | A | 749 | 6.634 | −14.299 | 5.824 | 1.00 | 39.58 |
| ATOM | 1722 | CB | PRO | A | 749 | 6.773 | −14.021 | 4.325 | 1.00 | 38.14 |
| ATOM | 1723 | CG | PRO | A | 749 | 7.069 | −15.367 | 3.745 | 1.00 | 38.10 |
| ATOM | 1724 | C | PRO | A | 749 | 5.176 | −14.417 | 6.274 | 1.00 | 39.62 |
| ATOM | 1725 | O | PRO | A | 749 | 4.601 | −13.447 | 6.770 | 1.00 | 39.95 |
| ATOM | 1726 | N | HIS | A | 750 | 4.571 | −15.588 | 6.108 | 1.00 | 32.50 |
| ATOM | 1727 | CA | HIS | A | 750 | 3.181 | −15.742 | 6.524 | 1.00 | 31.88 |
| ATOM | 1728 | CB | HIS | A | 750 | 2.562 | −17.016 | 5.946 | 1.00 | 30.66 |
| ATOM | 1729 | CG | HIS | A | 750 | 1.149 | −17.240 | 6.384 | 1.00 | 30.76 |
| ATOM | 1730 | CD2 | HIS | A | 750 | −0.008 | −16.667 | 5.981 | 1.00 | 30.83 |
| ATOM | 1731 | ND1 | HIS | A | 750 | 0.815 | −18.097 | 7.410 | 1.00 | 31.54 |
| ATOM | 1732 | CE1 | HIS | A | 750 | −0.488 | −18.041 | 7.621 | 1.00 | 31.23 |
| ATOM | 1733 | NE2 | HIS | A | 750 | −1.011 | −17.180 | 6.768 | 1.00 | 31.56 |
| ATOM | 1734 | C | HIS | A | 750 | 3.055 | −15.761 | 8.042 | 1.00 | 31.66 |
| ATOM | 1735 | O | HIS | A | 750 | 2.146 | −15.148 | 8.604 | 1.00 | 31.17 |
| ATOM | 1736 | N | GLU | A | 751 | 3.957 | −16.470 | 8.711 | 1.00 | 31.26 |
| ATOM | 1737 | CA | GLU | A | 751 | 3.913 | −16.519 | 10.165 | 1.00 | 31.20 |
| ATOM | 1738 | CB | GLU | A | 751 | 4.898 | −17.563 | 10.703 | 1.00 | 33.16 |
| ATOM | 1739 | CG | GLU | A | 751 | 4.444 | −19.000 | 10.455 | 1.00 | 33.17 |
| ATOM | 1740 | CD | GLU | A | 751 | 5.215 | −20.017 | 11.274 | 1.00 | 33.56 |
| ATOM | 1741 | OE1 | GLU | A | 751 | 5.432 | −19.772 | 12.478 | 1.00 | 33.10 |
| ATOM | 1742 | OE2 | GLU | A | 751 | 5.587 | −21.071 | 10.720 | 1.00 | 33.79 |
| ATOM | 1743 | C | GLU | A | 751 | 4.247 | −15.127 | 10.699 | 1.00 | 31.23 |
| ATOM | 1744 | O | GLU | A | 751 | 3.836 | −14.753 | 11.800 | 1.00 | 31.00 |
| ATOM | 1745 | N | LYS | A | 752 | 4.979 | −14.364 | 9.892 | 1.00 | 29.23 |
| ATOM | 1746 | CA | LYS | A | 752 | 5.372 | −13.008 | 10.239 | 1.00 | 29.88 |
| ATOM | 1747 | CB | LYS | A | 752 | 6.292 | −12.439 | 9.154 | 1.00 | 37.79 |
| ATOM | 1748 | CG | LYS | A | 752 | 6.867 | −11.058 | 9.453 | 1.00 | 38.37 |
| ATOM | 1749 | CD | LYS | A | 752 | 7.766 | −11.087 | 10.684 | 1.00 | 38.41 |
| ATOM | 1750 | CE | LYS | A | 752 | 8.526 | −9.783 | 10.861 | 1.00 | 38.61 |
| ATOM | 1751 | NZ | LYS | A | 752 | 9.473 | −9.521 | 9.748 | 1.00 | 38.19 |
| ATOM | 1752 | C | LYS | A | 752 | 4.143 | −12.120 | 10.373 | 1.00 | 29.16 |
| ATOM | 1753 | O | LYS | A | 752 | 3.921 | −11.510 | 11.425 | 1.00 | 29.42 |
| ATOM | 1754 | N | GLU | A | 753 | 3.341 | −12.029 | 9.315 | 1.00 | 30.11 |
| ATOM | 1755 | CA | GLU | A | 753 | 2.156 | −11.191 | 9.402 | 1.00 | 30.60 |
| ATOM | 1756 | CB | GLU | A | 753 | 1.467 | −11.040 | 8.038 | 1.00 | 54.85 |
| ATOM | 1757 | CG | GLU | A | 753 | 1.100 | −12.316 | 7.315 | 1.00 | 55.61 |
| ATOM | 1758 | CD | GLU | A | 753 | 0.501 | −12.034 | 5.942 | 1.00 | 55.82 |
| ATOM | 1759 | OE1 | GLU | A | 753 | 1.195 | −11.420 | 5.102 | 1.00 | 55.58 |
| ATOM | 1760 | OE2 | GLU | A | 753 | −0.663 | −12.420 | 5.705 | 1.00 | 55.80 |
| ATOM | 1761 | C | GLU | A | 753 | 1.201 | −11.736 | 10.458 | 1.00 | 29.96 |
| ATOM | 1762 | O | GLU | A | 753 | 0.476 | −10.973 | 11.094 | 1.00 | 30.36 |
| ATOM | 1763 | N | LEU | A | 754 | 1.215 | −13.050 | 10.668 | 1.00 | 25.09 |
| ATOM | 1764 | CA | LEU | A | 754 | 0.364 | −13.638 | 11.695 | 1.00 | 23.83 |
| ATOM | 1765 | CB | LEU | A | 754 | 0.510 | −15.160 | 11.719 | 1.00 | 25.34 |
| ATOM | 1766 | CG | LEU | A | 754 | −0.242 | −15.857 | 12.852 | 1.00 | 26.46 |
| ATOM | 1767 | CD1 | LEU | A | 754 | −1.730 | −15.558 | 12.759 | 1.00 | 25.32 |
| ATOM | 1768 | CD2 | LEU | A | 754 | 0.010 | −17.364 | 12.767 | 1.00 | 25.75 |
| ATOM | 1769 | C | LEU | A | 754 | 0.812 | −13.064 | 13.039 | 1.00 | 23.90 |
| ATOM | 1770 | O | LEU | A | 754 | −0.011 | −12.728 | 13.896 | 1.00 | 24.10 |
| ATOM | 1771 | N | PHE | A | 755 | 2.126 | −12.953 | 13.215 | 1.00 | 22.27 |
| ATOM | 1772 | CA | PHE | A | 755 | 2.673 | −12.405 | 14.444 | 1.00 | 23.27 |
| ATOM | 1773 | CB | PHE | A | 755 | 4.188 | −12.601 | 14.506 | 1.00 | 27.67 |
| ATOM | 1774 | CG | PHE | A | 755 | 4.811 | −11.977 | 15.715 | 1.00 | 27.49 |
| ATOM | 1775 | CD1 | PHE | A | 755 | 4.559 | −12.492 | 16.981 | 1.00 | 27.28 |
| ATOM | 1776 | CD2 | PHE | A | 755 | 5.589 | −10.834 | 15.596 | 1.00 | 26.66 |
| ATOM | 1777 | CE1 | PHE | A | 755 | 5.070 | −11.872 | 18.117 | 1.00 | 28.85 |
| ATOM | 1778 | CE2 | PHE | A | 755 | 6.108 | −10.200 | 16.727 | 1.00 | 27.67 |
| ATOM | 1779 | CZ | PHE | A | 755 | 5.849 | −10.718 | 17.987 | 1.00 | 27.51 |
| ATOM | 1780 | C | PHE | A | 755 | 2.352 | −10.914 | 14.590 | 1.00 | 23.26 |
| ATOM | 1781 | O | PHE | A | 755 | 1.932 | −10.469 | 15.665 | 1.00 | 22.65 |
| ATOM | 1782 | N | LEU | A | 756 | 2.553 | −10.148 | 13.520 | 1.00 | 21.99 |
| ATOM | 1783 | CA | LEU | A | 756 | 2.274 | −8.712 | 13.552 | 1.00 | 23.22 |
| ATOM | 1784 | CB | LEU | A | 756 | 2.593 | −8.059 | 12.206 | 1.00 | 24.32 |
| ATOM | 1785 | CG | LEU | A | 756 | 4.060 | −7.993 | 11.785 | 1.00 | 25.68 |
| ATOM | 1786 | CD1 | LEU | A | 756 | 4.163 | −7.476 | 10.364 | 1.00 | 24.86 |
| ATOM | 1787 | CD2 | LEU | A | 756 | 4.821 | −7.094 | 12.737 | 1.00 | 25.84 |
| ATOM | 1788 | C | LEU | A | 756 | 0.820 | −8.435 | 13.906 | 1.00 | 22.88 |
| ATOM | 1789 | O | LEU | A | 756 | 0.520 | −7.441 | 14.561 | 1.00 | 22.05 |
| ATOM | 1790 | N | ALA | A | 757 | −0.078 | −9.316 | 13.477 | 1.00 | 22.97 |
| ATOM | 1791 | CA | ALA | A | 757 | −1.498 | −9.152 | 13.770 | 1.00 | 22.24 |
| ATOM | 1792 | CB | ALA | A | 757 | −2.326 | −10.143 | 12.950 | 1.00 | 21.11 |
| ATOM | 1793 | C | ALA | A | 757 | −1.733 | −9.387 | 15.255 | 1.00 | 23.73 |
| ATOM | 1794 | O | ALA | A | 757 | −2.491 | −8.668 | 15.907 | 1.00 | 22.64 |
| ATOM | 1795 | N | MET | A | 758 | −1.080 | −10.407 | 15.794 | 1.00 | 17.51 |
| ATOM | 1796 | CA | MET | A | 758 | −1.234 | −10.717 | 17.198 | 1.00 | 18.28 |
| ATOM | 1797 | CB | MET | A | 758 | −0.581 | −12.072 | 17.503 | 1.00 | 27.85 |
| ATOM | 1798 | CG | MET | A | 758 | −1.181 | −13.255 | 16.720 | 1.00 | 27.03 |
| ATOM | 1799 | SD | MET | A | 758 | −2.826 | −13.794 | 17.241 | 1.00 | 29.72 |
| ATOM | 1800 | CE | MET | A | 758 | −3.778 | −13.473 | 15.747 | 1.00 | 28.03 |
| ATOM | 1801 | C | MET | A | 758 | −0.608 | −9.605 | 18.039 | 1.00 | 17.15 |
| ATOM | 1802 | O | MET | A | 758 | −1.088 | −9.319 | 19.137 | 1.00 | 18.07 |
| ATOM | 1803 | N | LEU | A | 759 | 0.447 | −8.979 | 17.515 | 1.00 | 17.47 |
| ATOM | 1804 | CA | LEU | A | 759 | 1.139 | −7.898 | 18.225 | 1.00 | 19.04 |
| ATOM | 1805 | CB | LEU | A | 759 | 2.418 | −7.482 | 17.491 | 1.00 | 19.37 |
| ATOM | 1806 | CG | LEU | A | 759 | 3.214 | −6.328 | 18.112 | 1.00 | 20.18 |
| ATOM | 1807 | CD1 | LEU | A | 759 | 3.610 | −6.669 | 19.550 | 1.00 | 21.45 |
| ATOM | 1808 | CD2 | LEU | A | 759 | 4.439 | −6.039 | 17.261 | 1.00 | 21.48 |
| ATOM | 1809 | C | LEU | A | 759 | 0.213 | −6.699 | 18.342 | 1.00 | 19.95 |
| ATOM | 1810 | O | LEU | A | 759 | 0.119 | −6.081 | 19.406 | 1.00 | 18.11 |
| ATOM | 1811 | N | MET | A | 760 | −0.454 | −6.367 | 17.240 | 1.00 | 17.24 |
| ATOM | 1812 | CA | MET | A | 760 | −1.393 | −5.240 | 17.251 | 1.00 | 17.90 |
| ATOM | 1813 | CB | MET | A | 760 | −2.032 | −5.063 | 15.874 | 1.00 | 18.91 |
| ATOM | 1814 | CG | MET | A | 760 | −1.140 | −4.422 | 14.835 | 1.00 | 19.09 |
| ATOM | 1815 | SD | MET | A | 760 | −0.717 | −2.696 | 15.216 | 1.00 | 18.56 |
| ATOM | 1816 | CE | MET | A | 760 | −2.370 | −1.939 | 15.227 | 1.00 | 17.91 |
| ATOM | 1817 | C | MET | A | 760 | −2.468 | −5.487 | 18.302 | 1.00 | 18.32 |
| ATOM | 1818 | O | MET | A | 760 | −2.808 | −4.590 | 19.072 | 1.00 | 15.93 |
| ATOM | 1819 | N | THR | A | 761 | −3.002 | −6.706 | 18.350 | 1.00 | 17.94 |
| ATOM | 1820 | CA | THR | A | 761 | −4.024 | −7.021 | 19.336 | 1.00 | 17.15 |
| ATOM | 1821 | CB | THR | A | 761 | −4.540 | −8.454 | 19.164 | 1.00 | 18.32 |
| ATOM | 1822 | OG1 | THR | A | 761 | −5.103 | −8.594 | 17.856 | 1.00 | 18.56 |
| ATOM | 1823 | CG2 | THR | A | 761 | −5.594 | −8.772 | 20.220 | 1.00 | 17.55 |
| ATOM | 1824 | C | THR | A | 761 | −3.446 | −6.880 | 20.746 | 1.00 | 17.99 |
| ATOM | 1825 | O | THR | A | 761 | −4.089 | −6.332 | 21.635 | 1.00 | 19.05 |
| ATOM | 1826 | N | ALA | A | 762 | −2.228 | −7.381 | 20.946 | 1.00 | 16.64 |
| ATOM | 1827 | CA | ALA | A | 762 | −1.590 | −7.298 | 22.255 | 1.00 | 16.82 |
| ATOM | 1828 | CB | ALA | A | 762 | −0.194 | −7.908 | 22.202 | 1.00 | 19.67 |
| ATOM | 1829 | C | ALA | A | 762 | −1.514 | −5.854 | 22.755 | 1.00 | 18.11 |
| ATOM | 1830 | O | ALA | A | 762 | −1.766 | −5.585 | 23.933 | 1.00 | 16.84 |
| ATOM | 1831 | N | CYS | A | 763 | −1.169 | −4.936 | 21.860 | 1.00 | 15.07 |
| ATOM | 1832 | CA | CYS | A | 763 | −1.081 | −3.523 | 22.216 | 1.00 | 16.86 |
| ATOM | 1833 | CB | CYS | A | 763 | −0.303 | −2.767 | 21.147 | 1.00 | 16.42 |
| ATOM | 1834 | SG | CYS | A | 763 | 1.450 | −3.220 | 21.036 | 1.00 | 16.68 |
| ATOM | 1835 | C | CYS | A | 763 | −2.469 | −2.904 | 22.372 | 1.00 | 18.36 |
| ATOM | 1836 | O | CYS | A | 763 | −2.705 | −2.091 | 23.269 | 1.00 | 15.92 |
| ATOM | 1837 | N | ASP | A | 764 | −3.385 | −3.289 | 21.494 | 1.00 | 17.47 |
| ATOM | 1838 | CA | ASP | A | 764 | −4.743 | −2.766 | 21.528 | 1.00 | 18.98 |
| ATOM | 1839 | CB | ASP | A | 764 | −5.539 | −3.360 | 20.360 | 1.00 | 18.28 |
| ATOM | 1840 | CG | ASP | A | 764 | −6.834 | −2.624 | 20.089 | 1.00 | 19.27 |
| ATOM | 1841 | OD1 | ASP | A | 764 | −7.670 | −3.161 | 19.333 | 1.00 | 22.03 |
| ATOM | 1842 | OD2 | ASP | A | 764 | −7.013 | −1.507 | 20.614 | 1.00 | 18.63 |
| ATOM | 1843 | C | ASP | A | 764 | −5.446 | −3.078 | 22.857 | 1.00 | 18.67 |
| ATOM | 1844 | O | ASP | A | 764 | −6.269 | −2.290 | 23.331 | 1.00 | 19.51 |
| ATOM | 1845 | N | LEU | A | 765 | −5.126 | −4.217 | 23.470 | 1.00 | 16.07 |
| ATOM | 1846 | CA | LEU | A | 765 | −5.771 | −4.596 | 24.726 | 1.00 | 16.91 |
| ATOM | 1847 | CB | LEU | A | 765 | −6.108 | −6.098 | 24.712 | 1.00 | 22.31 |
| ATOM | 1848 | CG | LEU | A | 765 | −6.925 | −6.653 | 23.540 | 1.00 | 22.32 |
| ATOM | 1849 | CD1 | LEU | A | 765 | −7.143 | −8.152 | 23.733 | 1.00 | 22.41 |
| ATOM | 1850 | CD2 | LEU | A | 765 | −8.260 | −5.940 | 23.451 | 1.00 | 22.60 |
| ATOM | 1851 | C | LEU | A | 765 | −4.911 | −4.288 | 25.953 | 1.00 | 17.63 |
| ATOM | 1852 | O | LEU | A | 765 | −5.250 | −4.699 | 27.062 | 1.00 | 18.64 |
| ATOM | 1853 | N | SER | A | 766 | −3.830 | −3.541 | 25.750 | 1.00 | 17.02 |
| ATOM | 1854 | CA | SER | A | 766 | −2.869 | −3.230 | 26.812 | 1.00 | 18.24 |
| ATOM | 1855 | CB | SER | A | 766 | −1.846 | −2.205 | 26.312 | 1.00 | 20.09 |
| ATOM | 1856 | OG | SER | A | 766 | −2.378 | −0.892 | 26.319 | 1.00 | 21.90 |
| ATOM | 1857 | C | SER | A | 766 | −3.400 | −2.775 | 28.171 | 1.00 | 17.35 |
| ATOM | 1858 | O | SER | A | 766 | −2.738 | −2.975 | 29.197 | 1.00 | 16.73 |
| ATOM | 1859 | N | ALA | A | 767 | −4.571 | −2.152 | 28.196 | 1.00 | 18.41 |
| ATOM | 1860 | CA | ALA | A | 767 | −5.113 | −1.682 | 29.466 | 1.00 | 19.58 |
| ATOM | 1861 | CB | ALA | A | 767 | −6.465 | −0.994 | 29.242 | 1.00 | 14.05 |
| ATOM | 1862 | C | ALA | A | 767 | −5.272 | −2.823 | 30.461 | 1.00 | 20.35 |
| ATOM | 1863 | O | ALA | A | 767 | −5.111 | −2.636 | 31.666 | 1.00 | 20.93 |
| ATOM | 1864 | N | ILE | A | 768 | −5.580 | −4.008 | 29.949 | 1.00 | 18.87 |
| ATOM | 1865 | CA | ILE | A | 768 | −5.789 | −5.176 | 30.786 | 1.00 | 20.62 |
| ATOM | 1866 | CB | ILE | A | 768 | −6.405 | −6.330 | 29.943 | 1.00 | 19.42 |
| ATOM | 1867 | CG2 | ILE | A | 768 | −5.323 | −7.008 | 29.112 | 1.00 | 20.41 |
| ATOM | 1868 | CG1 | ILE | A | 768 | −7.087 | −7.351 | 30.856 | 1.00 | 20.44 |
| ATOM | 1869 | CD1 | ILE | A | 768 | −8.333 | −6.831 | 31.548 | 1.00 | 20.90 |
| ATOM | 1870 | C | ILE | A | 768 | −4.499 | −5.657 | 31.466 | 1.00 | 20.39 |
| ATOM | 1871 | O | ILE | A | 768 | −4.543 | −6.544 | 32.307 | 1.00 | 21.21 |
| ATOM | 1872 | N | THR | A | 769 | −3.364 | −5.061 | 31.108 | 1.00 | 19.18 |
| ATOM | 1873 | CA | THR | A | 769 | −2.075 | −5.437 | 31.693 | 1.00 | 19.16 |
| ATOM | 1874 | CB | THR | A | 769 | −0.996 | −5.672 | 30.620 | 1.00 | 20.58 |
| ATOM | 1875 | OG1 | THR | A | 769 | −0.519 | −4.408 | 30.131 | 1.00 | 19.24 |
| ATOM | 1876 | CG2 | THR | A | 769 | −1.561 | −6.478 | 29.453 | 1.00 | 20.09 |
| ATOM | 1877 | C | THR | A | 769 | −1.496 | −4.379 | 32.637 | 1.00 | 21.13 |
| ATOM | 1878 | O | THR | A | 769 | −0.488 | −4.618 | 33.300 | 1.00 | 21.47 |
| ATOM | 1879 | N | LYS | A | 770 | −2.127 | −3.214 | 32.691 | 1.00 | 19.99 |
| ATOM | 1880 | CA | LYS | A | 770 | −1.634 | −2.120 | 33.527 | 1.00 | 20.68 |
| ATOM | 1881 | CB | LYS | A | 770 | −2.313 | −0.812 | 33.108 | 1.00 | 20.27 |
| ATOM | 1882 | CG | LYS | A | 770 | −2.103 | −0.409 | 31.660 | 1.00 | 19.98 |
| ATOM | 1883 | CD | LYS | A | 770 | −0.711 | 0.136 | 31.431 | 1.00 | 20.21 |
| ATOM | 1884 | CE | LYS | A | 770 | −0.502 | 0.464 | 29.960 | 1.00 | 20.34 |
| ATOM | 1885 | NZ | LYS | A | 770 | 0.829 | 1.065 | 29.701 | 1.00 | 21.28 |
| ATOM | 1886 | C | LYS | A | 770 | −1.861 | −2.330 | 35.026 | 1.00 | 19.97 |
| ATOM | 1887 | O | LYS | A | 770 | −2.628 | −3.204 | 35.437 | 1.00 | 20.86 |
| ATOM | 1888 | N | PRO | A | 771 | −1.186 | −1.521 | 35.866 | 1.00 | 23.24 |
| ATOM | 1889 | CD | PRO | A | 771 | −0.150 | −0.537 | 35.512 | 1.00 | 25.67 |
| ATOM | 1890 | CA | PRO | A | 771 | −1.329 | −1.615 | 37.321 | 1.00 | 24.06 |
| ATOM | 1891 | CB | PRO | A | 771 | −0.461 | −0.463 | 37.823 | 1.00 | 25.01 |
| ATOM | 1892 | CG | PRO | A | 771 | 0.636 | −0.421 | 36.809 | 1.00 | 24.38 |
| ATOM | 1893 | C | PRO | A | 771 | −2.804 | −1.429 | 37.672 | 1.00 | 23.94 |
| ATOM | 1894 | O | PRO | A | 771 | −3.529 | −0.723 | 36.970 | 1.00 | 23.81 |
| ATOM | 1895 | N | TRP | A | 772 | −3.236 | −2.057 | 38.759 | 1.00 | 22.70 |
| ATOM | 1896 | CA | TRP | A | 772 | −4.626 | −2.009 | 39.201 | 1.00 | 23.47 |
| ATOM | 1897 | CB | TRP | A | 772 | −4.721 | −2.480 | 40.652 | 1.00 | 31.03 |
| ATOM | 1898 | CG | TRP | A | 772 | −6.120 | −2.631 | 41.148 | 1.00 | 31.98 |
| ATOM | 1899 | CD2 | TRP | A | 772 | −7.205 | −3.295 | 40.484 | 1.00 | 31.41 |
| ATOM | 1900 | CE2 | TRP | A | 772 | −8.322 | −3.228 | 41.345 | 1.00 | 32.26 |
| ATOM | 1901 | CE3 | TRP | A | 772 | −7.342 | −3.940 | 39.246 | 1.00 | 31.94 |
| ATOM | 1902 | CD1 | TRP | A | 772 | −6.611 | −2.199 | 42.346 | 1.00 | 31.60 |
| ATOM | 1903 | NE1 | TRP | A | 772 | −7.930 | −2.553 | 42.471 | 1.00 | 31.93 |
| ATOM | 1904 | CZ2 | TRP | A | 772 | −9.565 | −3.785 | 41.009 | 1.00 | 32.15 |
| ATOM | 1905 | CZ3 | TRP | A | 772 | −8.579 | −4.493 | 38.912 | 1.00 | 31.45 |
| ATOM | 1906 | CH2 | TRP | A | 772 | −9.672 | −4.411 | 39.792 | 1.00 | 31.98 |
| ATOM | 1907 | C | TRP | A | 772 | −5.368 | −0.678 | 39.057 | 1.00 | 23.85 |
| ATOM | 1908 | O | TRP | A | 772 | −6.409 | −0.609 | 38.399 | 1.00 | 22.22 |
| ATOM | 1909 | N | PRO | A | 773 | −4.857 | 0.398 | 39.678 | 1.00 | 25.77 |
| ATOM | 1910 | CD | PRO | A | 773 | −3.583 | 0.541 | 40.403 | 1.00 | 32.22 |
| ATOM | 1911 | CA | PRO | A | 773 | −5.546 | 1.689 | 39.567 | 1.00 | 26.04 |
| ATOM | 1912 | CB | PRO | A | 773 | −4.609 | 2.650 | 40.304 | 1.00 | 32.61 |
| ATOM | 1913 | CG | PRO | A | 773 | −3.259 | 1.991 | 40.171 | 1.00 | 33.14 |
| ATOM | 1914 | C | PRO | A | 773 | −5.831 | 2.122 | 38.134 | 1.00 | 25.71 |
| ATOM | 1915 | O | PRO | A | 773 | −6.900 | 2.653 | 37.834 | 1.00 | 26.03 |
| ATOM | 1916 | N | ILE | A | 774 | −4.869 | 1.885 | 37.253 | 1.00 | 21.46 |
| ATOM | 1917 | CA | ILE | A | 774 | −5.008 | 2.236 | 35.850 | 1.00 | 20.45 |
| ATOM | 1918 | CB | ILE | A | 774 | −3.648 | 2.105 | 35.135 | 1.00 | 25.41 |
| ATOM | 1919 | CG2 | ILE | A | 774 | −3.790 | 2.436 | 33.654 | 1.00 | 25.27 |
| ATOM | 1920 | CG1 | ILE | A | 774 | −2.633 | 3.037 | 35.802 | 1.00 | 26.17 |
| ATOM | 1921 | CD1 | ILE | A | 774 | −1.205 | 2.807 | 35.355 | 1.00 | 25.47 |
| ATOM | 1922 | C | ILE | A | 774 | −6.038 | 1.329 | 35.173 | 1.00 | 19.51 |
| ATOM | 1923 | O | ILE | A | 774 | −6.928 | 1.804 | 34.471 | 1.00 | 18.75 |
| ATOM | 1924 | N | GLN | A | 775 | −5.925 | 0.021 | 35.388 | 1.00 | 22.02 |
| ATOM | 1925 | CA | GLN | A | 775 | −6.868 | −0.916 | 34.783 | 1.00 | 21.75 |
| ATOM | 1926 | CB | GLN | A | 775 | −6.491 | −2.361 | 35.135 | 1.00 | 20.94 |
| ATOM | 1927 | CG | GLN | A | 775 | −7.668 | −3.340 | 35.192 | 1.00 | 21.03 |
| ATOM | 1928 | CD | GLN | A | 775 | −8.395 | −3.531 | 33.864 | 1.00 | 21.36 |
| ATOM | 1929 | OE1 | GLN | A | 775 | −9.457 | −4.141 | 33.826 | 1.00 | 21.09 |
| ATOM | 1930 | NE2 | GLN | A | 775 | −7.826 | −3.018 | 32.779 | 1.00 | 21.40 |
| ATOM | 1931 | C | GLN | A | 775 | −8.300 | −0.626 | 35.219 | 1.00 | 21.97 |
| ATOM | 1932 | O | GLN | A | 775 | −9.216 | −0.637 | 34.402 | 1.00 | 21.91 |
| ATOM | 1933 | N | GLN | A | 776 | −8.486 | −0.372 | 36.510 | 1.00 | 21.52 |
| ATOM | 1934 | CA | GLN | A | 776 | −9.807 | −0.066 | 37.051 | 1.00 | 23.77 |
| ATOM | 1935 | CB | GLN | A | 776 | −9.709 | 0.263 | 38.537 | 1.00 | 38.83 |
| ATOM | 1936 | CG | GLN | A | 776 | −9.798 | −0.919 | 39.456 | 1.00 | 39.61 |
| ATOM | 1937 | CD | GLN | A | 776 | −9.747 | −0.503 | 40.910 | 1.00 | 39.43 |
| ATOM | 1938 | OE1 | GLN | A | 776 | −8.717 | −0.035 | 41.397 | 1.00 | 39.77 |
| ATOM | 1939 | NE2 | GLN | A | 776 | −10.865 | −0.662 | 41.610 | 1.00 | 39.38 |
| ATOM | 1940 | C | GLN | A | 776 | −10.420 | 1.133 | 36.350 | 1.00 | 23.07 |
| ATOM | 1941 | O | GLN | A | 776 | −11.582 | 1.106 | 35.942 | 1.00 | 24.26 |
| ATOM | 1942 | N | ARG | A | 777 | −9.628 | 2.190 | 36.231 | 1.00 | 23.71 |
| ATOM | 1943 | CA | ARG | A | 777 | −10.081 | 3.413 | 35.598 | 1.00 | 23.28 |
| ATOM | 1944 | CB | ARG | A | 777 | −8.993 | 4.489 | 35.692 | 1.00 | 27.59 |
| ATOM | 1945 | CG | ARG | A | 777 | −9.423 | 5.850 | 35.159 | 1.00 | 27.74 |
| ATOM | 1946 | CD | ARG | A | 777 | −8.305 | 6.873 | 35.280 | 1.00 | 27.86 |
| ATOM | 1947 | NE | ARG | A | 777 | −7.155 | 6.535 | 34.445 | 1.00 | 27.75 |
| ATOM | 1948 | CZ | ARG | A | 777 | −7.161 | 6.545 | 33.114 | 1.00 | 27.57 |
| ATOM | 1949 | NH1 | ARG | A | 777 | −8.257 | 6.881 | 32.448 | 1.00 | 28.23 |
| ATOM | 1950 | NH2 | ARG | A | 777 | −6.066 | 6.216 | 32.446 | 1.00 | 28.20 |
| ATOM | 1951 | C | ARG | A | 777 | −10.465 | 3.191 | 34.139 | 1.00 | 23.20 |
| ATOM | 1952 | O | ARG | A | 777 | −11.537 | 3.605 | 33.704 | 1.00 | 22.51 |
| ATOM | 1953 | N | ILE | A | 778 | −9.592 | 2.535 | 33.380 | 1.00 | 17.78 |
| ATOM | 1954 | CA | ILE | A | 778 | −9.884 | 2.297 | 31.972 | 1.00 | 17.96 |
| ATOM | 1955 | CB | ILE | A | 778 | −8.633 | 1.806 | 31.225 | 1.00 | 19.43 |
| ATOM | 1956 | CG2 | ILE | A | 778 | −8.967 | 1.520 | 29.764 | 1.00 | 18.09 |
| ATOM | 1957 | CG1 | ILE | A | 778 | −7.560 | 2.894 | 31.305 | 1.00 | 19.35 |
| ATOM | 1958 | CD1 | ILE | A | 778 | −6.214 | 2.505 | 30.738 | 1.00 | 19.60 |
| ATOM | 1959 | C | ILE | A | 778 | −11.038 | 1.323 | 31.796 | 1.00 | 18.56 |
| ATOM | 1960 | O | ILE | A | 778 | −11.872 | 1.502 | 30.903 | 1.00 | 18.79 |
| ATOM | 1961 | N | ALA | A | 779 | −11.106 | 0.309 | 32.654 | 1.00 | 20.16 |
| ATOM | 1962 | CA | ALA | A | 779 | −12.199 | −0.656 | 32.590 | 1.00 | 21.21 |
| ATOM | 1963 | CB | ALA | A | 779 | −12.066 | −1.680 | 33.708 | 1.00 | 24.43 |
| ATOM | 1964 | C | ALA | A | 779 | −13.531 | 0.091 | 32.712 | 1.00 | 21.19 |
| ATOM | 1965 | O | ALA | A | 779 | −14.519 | −0.280 | 32.085 | 1.00 | 20.38 |
| ATOM | 1966 | N | GLU | A | 780 | −13.558 | 1.147 | 33.518 | 1.00 | 20.96 |
| ATOM | 1967 | CA | GLU | A | 780 | −14.786 | 1.926 | 33.682 | 1.00 | 22.15 |
| ATOM | 1968 | CB | GLU | A | 780 | −14.652 | 2.894 | 34.863 | 1.00 | 51.37 |
| ATOM | 1969 | CG | GLU | A | 780 | −14.497 | 2.196 | 36.206 | 1.00 | 52.40 |
| ATOM | 1970 | CD | GLU | A | 780 | −14.449 | 3.163 | 37.373 | 1.00 | 53.03 |
| ATOM | 1971 | OE1 | GLU | A | 780 | −13.590 | 4.072 | 37.359 | 1.00 | 53.05 |
| ATOM | 1972 | OE2 | GLU | A | 780 | −15.266 | 3.013 | 38.306 | 1.00 | 52.97 |
| ATOM | 1973 | C | GLU | A | 780 | −15.147 | 2.696 | 32.407 | 1.00 | 21.53 |
| ATOM | 1974 | O | GLU | A | 780 | −16.326 | 2.918 | 32.127 | 1.00 | 20.90 |
| ATOM | 1975 | N | LEU | A | 781 | −14.134 | 3.105 | 31.642 | 1.00 | 18.46 |
| ATOM | 1976 | CA | LEU | A | 781 | −14.365 | 3.818 | 30.390 | 1.00 | 19.53 |
| ATOM | 1977 | CB | LEU | A | 781 | −13.050 | 4.399 | 29.847 | 1.00 | 20.51 |
| ATOM | 1978 | CG | LEU | A | 781 | −12.392 | 5.516 | 30.672 | 1.00 | 21.84 |
| ATOM | 1979 | CD1 | LEU | A | 781 | −11.131 | 6.026 | 29.976 | 1.00 | 21.36 |
| ATOM | 1980 | CD2 | LEU | A | 781 | −13.389 | 6.660 | 30.828 | 1.00 | 20.48 |
| ATOM | 1981 | C | LEU | A | 781 | −14.949 | 2.824 | 29.389 | 1.00 | 19.65 |
| ATOM | 1982 | O | LEU | A | 781 | −15.898 | 3.133 | 28.654 | 1.00 | 19.62 |
| ATOM | 1983 | N | VAL | A | 782 | −14.368 | 1.628 | 29.362 | 1.00 | 21.76 |
| ATOM | 1984 | CA | VAL | A | 782 | −14.837 | 0.569 | 28.474 | 1.00 | 22.54 |
| ATOM | 1985 | CB | VAL | A | 782 | −14.026 | −0.740 | 28.693 | 1.00 | 22.92 |
| ATOM | 1986 | CG1 | VAL | A | 782 | −14.659 | −1.889 | 27.931 | 1.00 | 21.63 |
| ATOM | 1987 | CG2 | VAL | A | 782 | −12.588 | −0.554 | 28.231 | 1.00 | 22.05 |
| ATOM | 1988 | C | VAL | A | 782 | −16.311 | 0.290 | 28.766 | 1.00 | 21.93 |
| ATOM | 1989 | O | VAL | A | 782 | −17.142 | 0.238 | 27.857 | 1.00 | 23.01 |
| ATOM | 1990 | N | ALA | A | 783 | −16.632 | 0.123 | 30.045 | 1.00 | 19.50 |
| ATOM | 1991 | CA | ALA | A | 783 | −18.000 | −0.166 | 30.445 | 1.00 | 21.17 |
| ATOM | 1992 | CB | ALA | A | 783 | −18.061 | −0.447 | 31.942 | 1.00 | 21.14 |
| ATOM | 1993 | C | ALA | A | 783 | −18.940 | 0.979 | 30.078 | 1.00 | 20.67 |
| ATOM | 1994 | O | ALA | A | 783 | −20.051 | 0.755 | 29.598 | 1.00 | 21.82 |
| ATOM | 1995 | N | THR | A | 784 | −18.490 | 2.209 | 30.291 | 1.00 | 23.13 |
| ATOM | 1996 | CA | THR | A | 784 | −19.312 | 3.362 | 29.962 | 1.00 | 23.26 |
| ATOM | 1997 | CB | THR | A | 784 | −18.577 | 4.668 | 30.312 | 1.00 | 24.94 |
| ATOM | 1998 | OG1 | THR | A | 784 | −18.379 | 4.726 | 31.731 | 1.00 | 25.01 |
| ATOM | 1999 | CG2 | THR | A | 784 | −19.379 | 5.877 | 29.864 | 1.00 | 24.89 |
| ATOM | 2000 | C | THR | A | 784 | −19.702 | 3.364 | 28.481 | 1.00 | 24.33 |
| ATOM | 2001 | O | THR | A | 784 | −20.881 | 3.543 | 28.130 | 1.00 | 22.64 |
| ATOM | 2002 | N | GLU | A | 785 | −18.720 | 3.153 | 27.612 | 1.00 | 19.82 |
| ATOM | 2003 | CA | GLU | A | 785 | −18.991 | 3.136 | 26.182 | 1.00 | 21.00 |
| ATOM | 2004 | CB | GLU | A | 785 | −17.678 | 2.998 | 25.387 | 1.00 | 21.06 |
| ATOM | 2005 | CG | GLU | A | 785 | −17.871 | 2.918 | 23.873 | 1.00 | 20.31 |
| ATOM | 2006 | CD | GLU | A | 785 | −16.575 | 3.080 | 23.081 | 1.00 | 21.05 |
| ATOM | 2007 | OE1 | GLU | A | 785 | −16.506 | 2.557 | 21.947 | 1.00 | 20.57 |
| ATOM | 2008 | OE2 | GLU | A | 785 | −15.637 | 3.740 | 23.572 | 1.00 | 18.86 |
| ATOM | 2009 | C | GLU | A | 785 | −19.965 | 2.006 | 25.846 | 1.00 | 20.29 |
| ATOM | 2010 | O | GLU | A | 785 | −20.905 | 2.211 | 25.083 | 1.00 | 20.32 |
| ATOM | 2011 | N | PHE | A | 786 | −19.753 | 0.824 | 26.422 | 1.00 | 21.26 |
| ATOM | 2012 | CA | PHE | A | 786 | −20.643 | −0.307 | 26.161 | 1.00 | 21.97 |
| ATOM | 2013 | CB | PHE | A | 786 | −20.185 | −1.567 | 26.901 | 1.00 | 26.06 |
| ATOM | 2014 | CG | PHE | A | 786 | −18.955 | −2.216 | 26.324 | 1.00 | 27.41 |
| ATOM | 2015 | CD1 | PHE | A | 786 | −18.629 | −2.067 | 24.978 | 1.00 | 27.06 |
| ATOM | 2016 | CD2 | PHE | A | 786 | −18.146 | −3.021 | 27.122 | 1.00 | 26.60 |
| ATOM | 2017 | CE1 | PHE | A | 786 | −17.519 | −2.711 | 24.437 | 1.00 | 27.27 |
| ATOM | 2018 | CE2 | PHE | A | 786 | −17.032 | −3.672 | 26.586 | 1.00 | 27.24 |
| ATOM | 2019 | CZ | PHE | A | 786 | −16.722 | −3.515 | 25.244 | 1.00 | 27.18 |
| ATOM | 2020 | C | PHE | A | 786 | −22.068 | 0.011 | 26.599 | 1.00 | 23.40 |
| ATOM | 2021 | O | PHE | A | 786 | −23.023 | −0.269 | 25.878 | 1.00 | 21.69 |
| ATOM | 2022 | N | PHE | A | 787 | −22.206 | 0.594 | 27.787 | 1.00 | 23.44 |
| ATOM | 2023 | CA | PHE | A | 787 | −23.530 | 0.926 | 28.297 | 1.00 | 22.70 |
| ATOM | 2024 | CB | PHE | A | 787 | −23.445 | 1.413 | 29.751 | 1.00 | 22.93 |
| ATOM | 2025 | CG | PHE | A | 787 | −22.945 | 0.370 | 30.719 | 1.00 | 21.91 |
| ATOM | 2026 | CD1 | PHE | A | 787 | −23.122 | −0.991 | 30.464 | 1.00 | 22.94 |
| ATOM | 2027 | CD2 | PHE | A | 787 | −22.335 | 0.749 | 31.910 | 1.00 | 23.07 |
| ATOM | 2028 | CE1 | PHE | A | 787 | −22.697 | −1.954 | 31.383 | 1.00 | 23.17 |
| ATOM | 2029 | CE2 | PHE | A | 787 | −21.907 | −0.201 | 32.833 | 1.00 | 22.50 |
| ATOM | 2030 | CZ | PHE | A | 787 | −22.087 | −1.555 | 32.572 | 1.00 | 23.33 |
| ATOM | 2031 | C | PHE | A | 787 | −24.215 | 1.968 | 27.417 | 1.00 | 23.79 |
| ATOM | 2032 | O | PHE | A | 787 | −25.431 | 1.906 | 27.205 | 1.00 | 21.92 |
| ATOM | 2033 | N | ASP | A | 788 | −23.442 | 2.912 | 26.884 | 1.00 | 23.70 |
| ATOM | 2034 | CA | ASP | A | 788 | −24.021 | 3.923 | 26.010 | 1.00 | 25.33 |
| ATOM | 2035 | CB | ASP | A | 788 | −22.978 | 4.959 | 25.591 | 1.00 | 54.55 |
| ATOM | 2036 | CG | ASP | A | 788 | −23.605 | 6.296 | 25.233 | 1.00 | 55.82 |
| ATOM | 2037 | OD1 | ASP | A | 788 | −24.157 | 6.955 | 26.141 | 1.00 | 56.10 |
| ATOM | 2038 | OD2 | ASP | A | 788 | −23.558 | 6.686 | 24.046 | 1.00 | 55.97 |
| ATOM | 2039 | C | ASP | A | 788 | −24.571 | 3.216 | 24.774 | 1.00 | 24.36 |
| ATOM | 2040 | O | ASP | A | 788 | −25.646 | 3.551 | 24.283 | 1.00 | 25.06 |
| ATOM | 2041 | N | GLN | A | 789 | −23.837 | 2.235 | 24.257 | 1.00 | 20.19 |
| ATOM | 2042 | CA | GLN | A | 789 | −24.350 | 1.522 | 23.101 | 1.00 | 19.43 |
| ATOM | 2043 | CB | GLN | A | 789 | −23.340 | 0.508 | 22.566 | 1.00 | 19.05 |
| ATOM | 2044 | CG | GLN | A | 789 | −23.987 | −0.433 | 21.563 | 1.00 | 19.55 |
| ATOM | 2045 | CD | GLN | A | 789 | −23.011 | −1.268 | 20.781 | 1.00 | 19.48 |
| ATOM | 2046 | OE1 | GLN | A | 789 | −23.388 | −2.299 | 20.222 | 1.00 | 20.82 |
| ATOM | 2047 | NE2 | GLN | A | 789 | −21.755 | −0.833 | 20.718 | 1.00 | 18.14 |
| ATOM | 2048 | C | GLN | A | 789 | −25.629 | 0.802 | 23.503 | 1.00 | 20.46 |
| ATOM | 2049 | O | GLN | A | 789 | −26.586 | 0.735 | 22.729 | 1.00 | 20.11 |
| ATOM | 2050 | N | GLY | A | 790 | −25.639 | 0.266 | 24.721 | 1.00 | 22.31 |
| ATOM | 2051 | CA | GLY | A | 790 | −26.810 | −0.436 | 25.216 | 1.00 | 24.09 |
| ATOM | 2052 | C | GLY | A | 790 | −28.031 | 0.463 | 25.175 | 1.00 | 25.10 |
| ATOM | 2053 | O | GLY | A | 790 | −29.115 | 0.025 | 24.783 | 1.00 | 24.31 |
| ATOM | 2054 | N | ASP | A | 791 | −27.857 | 1.722 | 25.573 | 1.00 | 25.00 |
| ATOM | 2055 | CA | ASP | A | 791 | −28.963 | 2.683 | 25.568 | 1.00 | 25.93 |
| ATOM | 2056 | CB | ASP | A | 791 | −28.525 | 4.026 | 26.160 | 1.00 | 29.26 |
| ATOM | 2057 | CG | ASP | A | 791 | −28.107 | 3.918 | 27.611 | 1.00 | 29.25 |
| ATOM | 2058 | OD1 | ASP | A | 791 | −28.575 | 2.987 | 28.297 | 1.00 | 29.11 |
| ATOM | 2059 | OD2 | ASP | A | 791 | −27.323 | 4.778 | 28.068 | 1.00 | 30.04 |
| ATOM | 2060 | C | ASP | A | 791 | −29.464 | 2.909 | 24.143 | 1.00 | 25.33 |
| ATOM | 2061 | O | ASP | A | 791 | −30.669 | 3.029 | 23.907 | 1.00 | 26.34 |
| ATOM | 2062 | N | ARG | A | 792 | −28.527 | 2.984 | 23.205 | 1.00 | 25.90 |
| ATOM | 2063 | CA | ARG | A | 792 | −28.842 | 3.185 | 21.794 | 1.00 | 25.99 |
| ATOM | 2064 | CB | ARG | A | 792 | −27.546 | 3.326 | 20.990 | 1.00 | 34.02 |
| ATOM | 2065 | CG | ARG | A | 792 | −26.801 | 4.633 | 21.228 | 1.00 | 34.62 |
| ATOM | 2066 | CD | ARG | A | 792 | −27.310 | 5.716 | 20.300 | 1.00 | 35.57 |
| ATOM | 2067 | NE | ARG | A | 792 | −26.820 | 5.533 | 18.935 | 1.00 | 35.28 |
| ATOM | 2068 | CZ | ARG | A | 792 | −25.591 | 5.845 | 18.537 | 1.00 | 34.46 |
| ATOM | 2069 | NH1 | ARG | A | 792 | −24.726 | 6.358 | 19.403 | 1.00 | 34.52 |
| ATOM | 2070 | NH2 | ARG | A | 792 | −25.225 | 5.647 | 17.278 | 1.00 | 34.52 |
| ATOM | 2071 | C | ARG | A | 792 | −29.645 | 2.005 | 21.261 | 1.00 | 26.14 |
| ATOM | 2072 | O | ARG | A | 792 | −30.634 | 2.179 | 20.553 | 1.00 | 26.08 |
| ATOM | 2073 | N | GLU | A | 793 | −29.219 | 0.797 | 21.608 | 1.00 | 25.23 |
| ATOM | 2074 | CA | GLU | A | 793 | −29.910 | −0.396 | 21.141 | 1.00 | 25.15 |
| ATOM | 2075 | CB | GLU | A | 793 | −29.156 | −1.650 | 21.584 | 1.00 | 28.67 |
| ATOM | 2076 | CG | GLU | A | 793 | −27.715 | −1.688 | 21.120 | 1.00 | 29.21 |
| ATOM | 2077 | CD | GLU | A | 793 | −27.005 | −2.958 | 21.543 | 1.00 | 29.03 |
| ATOM | 2078 | OE1 | GLU | A | 793 | −27.219 | −3.406 | 22.689 | 1.00 | 28.67 |
| ATOM | 2079 | OE2 | GLU | A | 793 | −26.222 | −3.501 | 20.737 | 1.00 | 28.60 |
| ATOM | 2080 | C | GLU | A | 793 | −31.343 | −0.444 | 21.660 | 1.00 | 26.11 |
| ATOM | 2081 | O | GLU | A | 793 | −32.254 | −0.858 | 20.945 | 1.00 | 25.51 |
| ATOM | 2082 | N | ARG | A | 794 | −31.546 | −0.022 | 22.904 | 1.00 | 30.47 |
| ATOM | 2083 | CA | ARG | A | 794 | −32.887 | −0.041 | 23.477 | 1.00 | 30.88 |
| ATOM | 2084 | CB | ARG | A | 794 | −32.861 | 0.402 | 24.945 | 1.00 | 42.43 |
| ATOM | 2085 | CG | ARG | A | 794 | −31.776 | −0.241 | 25.786 | 1.00 | 42.99 |
| ATOM | 2086 | CD | ARG | A | 794 | −31.978 | 0.056 | 27.266 | 1.00 | 43.11 |
| ATOM | 2087 | NE | ARG | A | 794 | −32.839 | −0.939 | 27.893 | 1.00 | 43.32 |
| ATOM | 2088 | CZ | ARG | A | 794 | −32.425 | −1.820 | 28.798 | 1.00 | 42.83 |
| ATOM | 2089 | NH1 | ARG | A | 794 | −31.158 | −1.826 | 29.194 | 1.00 | 43.06 |
| ATOM | 2090 | NH2 | ARG | A | 794 | −33.274 | −2.708 | 29.295 | 1.00 | 43.25 |
| ATOM | 2091 | C | ARG | A | 794 | −33.818 | 0.886 | 22.701 | 1.00 | 31.12 |
| ATOM | 2092 | O | ARG | A | 794 | −34.972 | 0.547 | 22.451 | 1.00 | 30.32 |
| ATOM | 2093 | N | LYS | A | 795 | −33.307 | 2.050 | 22.314 | 1.00 | 28.47 |
| ATOM | 2094 | CA | LYS | A | 795 | −34.111 | 3.036 | 21.597 | 1.00 | 30.19 |
| ATOM | 2095 | CB | LYS | A | 795 | −33.538 | 4.438 | 21.833 | 1.00 | 43.10 |
| ATOM | 2096 | CG | LYS | A | 795 | −33.084 | 4.688 | 23.262 | 1.00 | 43.51 |
| ATOM | 2097 | CD | LYS | A | 795 | −32.642 | 6.129 | 23.482 | 1.00 | 44.03 |
| ATOM | 2098 | CE | LYS | A | 795 | −33.794 | 6.991 | 23.976 | 1.00 | 43.69 |
| ATOM | 2099 | NZ | LYS | A | 795 | −34.961 | 6.964 | 23.051 | 1.00 | 43.61 |
| ATOM | 2100 | C | LYS | A | 795 | −34.238 | 2.799 | 20.093 | 1.00 | 29.44 |
| ATOM | 2101 | O | LYS | A | 795 | −35.341 | 2.832 | 19.545 | 1.00 | 29.39 |
| ATOM | 2102 | N | GLU | A | 796 | −33.110 | 2.558 | 19.433 | 1.00 | 32.97 |
| ATOM | 2103 | CA | GLU | A | 796 | −33.080 | 2.358 | 17.987 | 1.00 | 33.23 |
| ATOM | 2104 | CB | GLU | A | 796 | −31.723 | 2.803 | 17.443 | 1.00 | 31.41 |
| ATOM | 2105 | CG | GLU | A | 796 | −31.265 | 4.158 | 17.957 | 1.00 | 31.18 |
| ATOM | 2106 | CD | GLU | A | 796 | −29.913 | 4.573 | 17.401 | 1.00 | 31.42 |
| ATOM | 2107 | OE1 | GLU | A | 796 | −29.334 | 5.558 | 17.904 | 1.00 | 31.78 |
| ATOM | 2108 | OE2 | GLU | A | 796 | −29.432 | 3.917 | 16.455 | 1.00 | 32.26 |
| ATOM | 2109 | C | GLU | A | 796 | −33.376 | 0.939 | 17.496 | 1.00 | 33.01 |
| ATOM | 2110 | O | GLU | A | 796 | −33.778 | 0.750 | 16.347 | 1.00 | 33.78 |
| ATOM | 2111 | N | LEU | A | 797 | −33.170 | −0.058 | 18.347 | 1.00 | 29.85 |
| ATOM | 2112 | CA | LEU | A | 797 | −33.424 | −1.438 | 17.952 | 1.00 | 29.25 |
| ATOM | 2113 | CB | LEU | A | 797 | −32.134 | −2.261 | 18.059 | 1.00 | 32.07 |
| ATOM | 2114 | CG | LEU | A | 797 | −30.951 | −1.828 | 17.185 | 1.00 | 32.45 |
| ATOM | 2115 | CD1 | LEU | A | 797 | −29.759 | −2.731 | 17.475 | 1.00 | 32.42 |
| ATOM | 2116 | CD2 | LEU | A | 797 | −31.325 | −1.904 | 15.709 | 1.00 | 32.29 |
| ATOM | 2117 | C | LEU | A | 797 | −34.513 | −2.073 | 18.807 | 1.00 | 29.52 |
| ATOM | 2118 | O | LEU | A | 797 | −34.954 | −3.192 | 18.534 | 1.00 | 29.88 |
| ATOM | 2119 | N | ASN | A | 798 | −34.940 | −1.348 | 19.839 | 1.00 | 31.24 |
| ATOM | 2120 | CA | ASN | A | 798 | −35.972 | −1.815 | 20.757 | 1.00 | 31.79 |
| ATOM | 2121 | CB | ASN | A | 798 | −37.332 | −1.805 | 20.062 | 1.00 | 46.67 |
| ATOM | 2122 | CG | ASN | A | 798 | −37.975 | −0.439 | 20.083 | 1.00 | 47.32 |
| ATOM | 2123 | OD1 | ASN | A | 798 | −38.405 | 0.038 | 21.136 | 1.00 | 47.67 |
| ATOM | 2124 | ND2 | ASN | A | 798 | −38.037 | 0.208 | 18.924 | 1.00 | 47.41 |
| ATOM | 2125 | C | ASN | A | 798 | −35.694 | −3.189 | 21.338 | 1.00 | 31.17 |
| ATOM | 2126 | O | ASN | A | 798 | −36.593 | −4.016 | 21.468 | 1.00 | 30.69 |
| ATOM | 2127 | N | ILE | A | 799 | −34.438 | −3.427 | 21.694 | 1.00 | 29.19 |
| ATOM | 2128 | CA | ILE | A | 799 | −34.046 | −4.701 | 22.274 | 1.00 | 29.70 |
| ATOM | 2129 | CB | ILE | A | 799 | −33.026 | −5.444 | 21.379 | 1.00 | 28.41 |
| ATOM | 2130 | CG2 | ILE | A | 799 | −33.660 | −5.795 | 20.036 | 1.00 | 29.14 |
| ATOM | 2131 | CG1 | ILE | A | 799 | −31.781 | −4.571 | 21.191 | 1.00 | 28.71 |
| ATOM | 2132 | CD1 | ILE | A | 799 | −30.632 | −5.251 | 20.494 | 1.00 | 28.52 |
| ATOM | 2133 | C | ILE | A | 799 | −33.389 | −4.463 | 23.627 | 1.00 | 29.70 |
| ATOM | 2134 | O | ILE | A | 799 | −32.889 | −3.370 | 23.904 | 1.00 | 29.11 |
| ATOM | 2135 | N | GLU | A | 800 | −33.410 | −5.481 | 24.477 | 1.00 | 31.12 |
| ATOM | 2136 | CA | GLU | A | 800 | −32.764 | −5.369 | 25.775 | 1.00 | 32.32 |
| ATOM | 2137 | CB | GLU | A | 800 | −33.322 | −6.394 | 26.768 | 1.00 | 47.83 |
| ATOM | 2138 | CG | GLU | A | 800 | −32.572 | −6.418 | 28.099 | 1.00 | 48.49 |
| ATOM | 2139 | CD | GLU | A | 800 | −32.999 | −7.559 | 29.007 | 1.00 | 48.67 |
| ATOM | 2140 | OE1 | GLU | A | 800 | −32.393 | −7.713 | 30.091 | 1.00 | 48.86 |
| ATOM | 2141 | OE2 | GLU | A | 800 | −33.938 | −8.299 | 28.640 | 1.00 | 48.41 |
| ATOM | 2142 | C | GLU | A | 800 | −31.309 | −5.692 | 25.474 | 1.00 | 31.85 |
| ATOM | 2143 | O | GLU | A | 800 | −31.009 | −6.731 | 24.888 | 1.00 | 31.72 |
| ATOM | 2144 | N | PRO | A | 801 | −30.386 | −4.801 | 25.855 | 1.00 | 32.29 |
| ATOM | 2145 | CD | PRO | A | 801 | −30.534 | −3.557 | 26.629 | 1.00 | 32.84 |
| ATOM | 2146 | CA | PRO | A | 801 | −28.976 | −5.083 | 25.578 | 1.00 | 32.39 |
| ATOM | 2147 | CB | PRO | A | 801 | −28.281 | −3.798 | 26.019 | 1.00 | 33.48 |
| ATOM | 2148 | CG | PRO | A | 801 | −29.143 | −3.351 | 27.168 | 1.00 | 33.84 |
| ATOM | 2149 | C | PRO | A | 801 | −28.462 | −6.308 | 26.323 | 1.00 | 31.89 |
| ATOM | 2150 | O | PRO | A | 801 | −29.056 | −6.752 | 27.312 | 1.00 | 31.79 |
| ATOM | 2151 | N | THR | A | 802 | −27.362 | −6.863 | 25.826 | 1.00 | 28.76 |
| ATOM | 2152 | CA | THR | A | 802 | −26.739 | −8.016 | 26.456 | 1.00 | 28.15 |
| ATOM | 2153 | CB | THR | A | 802 | −25.626 | −8.603 | 25.569 | 1.00 | 44.26 |
| ATOM | 2154 | OG1 | THR | A | 802 | −25.026 | −9.721 | 26.233 | 1.00 | 45.50 |
| ATOM | 2155 | CG2 | THR | A | 802 | −24.561 | −7.555 | 25.282 | 1.00 | 44.69 |
| ATOM | 2156 | C | THR | A | 802 | −26.139 | −7.521 | 27.767 | 1.00 | 28.29 |
| ATOM | 2157 | O | THR | A | 802 | −25.956 | −6.317 | 27.960 | 1.00 | 27.03 |
| ATOM | 2158 | N | ASP | A | 803 | −25.843 | −8.448 | 28.669 | 1.00 | 28.44 |
| ATOM | 2159 | CA | ASP | A | 803 | −25.275 | −8.094 | 29.963 | 1.00 | 28.64 |
| ATOM | 2160 | CB | ASP | A | 803 | −24.779 | −9.358 | 30.667 | 1.00 | 34.48 |
| ATOM | 2161 | CG | ASP | A | 803 | −25.893 | −10.098 | 31.379 | 1.00 | 34.81 |
| ATOM | 2162 | OD1 | ASP | A | 803 | −27.045 | −10.054 | 30.893 | 1.00 | 34.90 |
| ATOM | 2163 | OD2 | ASP | A | 803 | −25.611 | −10.730 | 32.420 | 1.00 | 35.01 |
| ATOM | 2164 | C | ASP | A | 803 | −24.153 | −7.063 | 29.906 | 1.00 | 28.44 |
| ATOM | 2165 | O | ASP | A | 803 | −24.176 | −6.074 | 30.635 | 1.00 | 28.48 |
| ATOM | 2166 | N | LEU | A | 804 | −23.177 | −7.275 | 29.031 | 1.00 | 25.97 |
| ATOM | 2167 | CA | LEU | A | 804 | −22.058 | −6.348 | 28.952 | 1.00 | 26.25 |
| ATOM | 2168 | CB | LEU | A | 804 | −20.951 | −6.937 | 28.075 | 1.00 | 47.64 |
| ATOM | 2169 | CG | LEU | A | 804 | −21.304 | −7.412 | 26.671 | 1.00 | 48.05 |
| ATOM | 2170 | CD1 | LEU | A | 804 | −21.264 | −6.244 | 25.708 | 1.00 | 48.53 |
| ATOM | 2171 | CD2 | LEU | A | 804 | −20.301 | −8.466 | 26.242 | 1.00 | 48.25 |
| ATOM | 2172 | C | LEU | A | 804 | −22.415 | −4.938 | 28.501 | 1.00 | 26.00 |
| ATOM | 2173 | O | LEU | A | 804 | −21.654 | −4.007 | 28.728 | 1.00 | 25.68 |
| ATOM | 2174 | N | MET | A | 805 | −23.576 | −4.763 | 27.881 | 1.00 | 25.47 |
| ATOM | 2175 | CA | MET | A | 805 | −23.971 | −3.427 | 27.445 | 1.00 | 25.62 |
| ATOM | 2176 | CB | MET | A | 805 | −24.246 | −3.422 | 25.940 | 1.00 | 32.20 |
| ATOM | 2177 | CG | MET | A | 805 | −22.992 | −3.634 | 25.113 | 1.00 | 31.73 |
| ATOM | 2178 | SD | MET | A | 805 | −23.263 | −3.668 | 23.345 | 1.00 | 32.78 |
| ATOM | 2179 | CE | MET | A | 805 | −21.557 | −3.485 | 22.751 | 1.00 | 31.70 |
| ATOM | 2180 | C | MET | A | 805 | −25.186 | −2.920 | 28.209 | 1.00 | 24.74 |
| ATOM | 2181 | O | MET | A | 805 | −25.808 | −1.932 | 27.813 | 1.00 | 25.55 |
| ATOM | 2182 | N | ASN | A | 806 | −25.502 | −3.596 | 29.311 | 1.00 | 23.96 |
| ATOM | 2183 | CA | ASN | A | 806 | −26.645 | −3.239 | 30.151 | 1.00 | 25.57 |
| ATOM | 2184 | CB | ASN | A | 806 | −27.467 | −4.495 | 30.463 | 1.00 | 28.17 |
| ATOM | 2185 | CG | ASN | A | 806 | −28.738 | −4.190 | 31.241 | 1.00 | 28.82 |
| ATOM | 2186 | OD1 | ASN | A | 806 | −28.985 | −3.049 | 31.628 | 1.00 | 28.36 |
| ATOM | 2187 | ND2 | ASN | A | 806 | −29.550 | −5.216 | 31.474 | 1.00 | 28.76 |
| ATOM | 2188 | C | ASN | A | 806 | −26.159 | −2.592 | 31.448 | 1.00 | 25.52 |
| ATOM | 2189 | O | ASN | A | 806 | −25.648 | −3.273 | 32.338 | 1.00 | 25.16 |
| ATOM | 2190 | N | ARG | A | 807 | −26.321 | −1.276 | 31.544 | 1.00 | 25.21 |
| ATOM | 2191 | CA | ARG | A | 807 | −25.892 | −0.512 | 32.717 | 1.00 | 25.42 |
| ATOM | 2192 | CB | ARG | A | 807 | −26.254 | 0.963 | 32.532 | 1.00 | 30.28 |
| ATOM | 2193 | CG | ARG | A | 807 | −25.516 | 1.919 | 33.450 | 1.00 | 31.10 |
| ATOM | 2194 | CD | ARG | A | 807 | −26.051 | 3.331 | 33.290 | 1.00 | 31.22 |
| ATOM | 2195 | NE | ARG | A | 807 | −26.092 | 3.752 | 31.892 | 1.00 | 31.51 |
| ATOM | 2196 | CZ | ARG | A | 807 | −25.040 | 4.157 | 31.192 | 1.00 | 31.00 |
| ATOM | 2197 | NH1 | ARG | A | 807 | −23.839 | 4.205 | 31.753 | 1.00 | 32.13 |
| ATOM | 2198 | NH2 | ARG | A | 807 | −25.191 | 4.516 | 29.923 | 1.00 | 30.26 |
| ATOM | 2199 | C | ARG | A | 807 | −26.528 | −1.030 | 34.002 | 1.00 | 27.00 |
| ATOM | 2200 | O | ARG | A | 807 | −25.916 | −0.988 | 35.073 | 1.00 | 26.42 |
| ATOM | 2201 | N | GLU | A | 808 | −27.762 | −1.508 | 33.895 | 1.00 | 29.47 |
| ATOM | 2202 | CA | GLU | A | 808 | −28.477 | −2.031 | 35.053 | 1.00 | 30.52 |
| ATOM | 2203 | CB | GLU | A | 808 | −29.914 | −2.372 | 34.659 | 1.00 | 44.58 |
| ATOM | 2204 | CG | GLU | A | 808 | −30.775 | −1.147 | 34.408 | 1.00 | 45.18 |
| ATOM | 2205 | CD | GLU | A | 808 | −32.132 | −1.492 | 33.837 | 1.00 | 45.51 |
| ATOM | 2206 | OE1 | GLU | A | 808 | −32.761 | −2.451 | 34.332 | 1.00 | 45.53 |
| ATOM | 2207 | OE2 | GLU | A | 808 | −32.573 | −0.796 | 32.898 | 1.00 | 45.65 |
| ATOM | 2208 | C | GLU | A | 808 | −27.780 | −3.261 | 35.629 | 1.00 | 30.44 |
| ATOM | 2209 | O | GLU | A | 808 | −27.875 | −3.542 | 36.828 | 1.00 | 30.33 |
| ATOM | 2210 | N | LYS | A | 809 | −27.077 | −3.985 | 34.765 | 1.00 | 31.29 |
| ATOM | 2211 | CA | LYS | A | 809 | −26.355 | −5.190 | 35.162 | 1.00 | 31.25 |
| ATOM | 2212 | CB | LYS | A | 809 | −26.522 | −6.279 | 34.092 | 1.00 | 41.16 |
| ATOM | 2213 | CG | LYS | A | 809 | −27.951 | −6.773 | 33.888 | 1.00 | 41.47 |
| ATOM | 2214 | CD | LYS | A | 809 | −28.391 | −7.716 | 34.995 | 1.00 | 41.55 |
| ATOM | 2215 | CE | LYS | A | 809 | −27.714 | −9.075 | 34.885 | 1.00 | 41.48 |
| ATOM | 2216 | NZ | LYS | A | 809 | −28.230 | −9.865 | 33.733 | 1.00 | 41.41 |
| ATOM | 2217 | C | LYS | A | 809 | −24.870 | −4.879 | 35.338 | 1.00 | 30.98 |
| ATOM | 2218 | O | LYS | A | 809 | −24.023 | −5.744 | 35.129 | 1.00 | 30.77 |
| ATOM | 2219 | N | LYS | A | 810 | −24.551 | −3.646 | 35.721 | 1.00 | 26.58 |
| ATOM | 2220 | CA | LYS | A | 810 | −23.155 | −3.259 | 35.901 | 1.00 | 26.99 |
| ATOM | 2221 | CB | LYS | A | 810 | −23.050 | −1.768 | 36.235 | 1.00 | 43.46 |
| ATOM | 2222 | CG | LYS | A | 810 | −23.626 | −1.387 | 37.587 | 1.00 | 44.21 |
| ATOM | 2223 | CD | LYS | A | 810 | −23.413 | 0.090 | 37.878 | 1.00 | 44.21 |
| ATOM | 2224 | CE | LYS | A | 810 | −23.942 | 0.459 | 39.256 | 1.00 | 44.19 |
| ATOM | 2225 | NZ | LYS | A | 810 | −23.718 | 1.897 | 39.566 | 1.00 | 44.47 |
| ATOM | 2226 | C | LYS | A | 810 | −22.462 | −4.077 | 36.997 | 1.00 | 26.09 |
| ATOM | 2227 | O | LYS | A | 810 | −21.234 | −4.128 | 37.060 | 1.00 | 26.59 |
| ATOM | 2228 | N | ASN | A | 811 | −23.250 | −4.720 | 37.852 | 1.00 | 29.32 |
| ATOM | 2229 | CA | ASN | A | 811 | −22.693 | −5.526 | 38.934 | 1.00 | 29.87 |
| ATOM | 2230 | CB | ASN | A | 811 | −23.812 | −6.017 | 39.858 | 1.00 | 45.97 |
| ATOM | 2231 | CG | ASN | A | 811 | −24.632 | −7.135 | 39.240 | 1.00 | 46.38 |
| ATOM | 2232 | OD1 | ASN | A | 811 | −24.137 | −8.247 | 39.042 | 1.00 | 46.84 |
| ATOM | 2233 | ND2 | ASN | A | 811 | −25.887 | −6.847 | 38.927 | 1.00 | 46.45 |
| ATOM | 2234 | C | ASN | A | 811 | −21.931 | −6.730 | 38.385 | 1.00 | 29.68 |
| ATOM | 2235 | O | ASN | A | 811 | −21.228 | −7.425 | 39.121 | 1.00 | 29.10 |
| ATOM | 2236 | N | LYS | A | 812 | −22.075 | −6.970 | 37.086 | 1.00 | 24.91 |
| ATOM | 2237 | CA | LYS | A | 812 | −21.418 | −8.100 | 36.438 | 1.00 | 25.57 |
| ATOM | 2238 | CB | LYS | A | 812 | −22.292 | −8.623 | 35.295 | 1.00 | 45.40 |
| ATOM | 2239 | CG | LYS | A | 812 | −22.850 | −10.016 | 35.509 | 1.00 | 46.25 |
| ATOM | 2240 | CD | LYS | A | 812 | −23.811 | −10.060 | 36.678 | 1.00 | 46.19 |
| ATOM | 2241 | CE | LYS | A | 812 | −24.429 | −11.439 | 36.816 | 1.00 | 46.23 |
| ATOM | 2242 | NZ | LYS | A | 812 | −23.387 | −12.487 | 37.001 | 1.00 | 46.38 |
| ATOM | 2243 | C | LYS | A | 812 | −20.025 | −7.789 | 35.886 | 1.00 | 24.76 |
| ATOM | 2244 | O | LYS | A | 812 | −19.340 | −8.688 | 35.401 | 1.00 | 24.68 |
| ATOM | 2245 | N | ILE | A | 813 | −19.598 | −6.534 | 35.963 | 1.00 | 26.65 |
| ATOM | 2246 | CA | ILE | A | 813 | −18.294 | −6.162 | 35.424 | 1.00 | 26.72 |
| ATOM | 2247 | CB | ILE | A | 813 | −18.008 | −4.656 | 35.630 | 1.00 | 25.86 |
| ATOM | 2248 | CG2 | ILE | A | 813 | −16.564 | −4.337 | 35.260 | 1.00 | 25.72 |
| ATOM | 2249 | CG1 | ILE | A | 813 | −18.955 | −3.830 | 34.751 | 1.00 | 25.85 |
| ATOM | 2250 | CD1 | ILE | A | 813 | −18.880 | −2.335 | 34.984 | 1.00 | 26.75 |
| ATOM | 2251 | C | ILE | A | 813 | −17.123 | −6.986 | 35.957 | 1.00 | 26.25 |
| ATOM | 2252 | O | ILE | A | 813 | −16.271 | −7.426 | 35.185 | 1.00 | 25.98 |
| ATOM | 2253 | N | PRO | A | 814 | −17.055 | −7.205 | 37.280 | 1.00 | 22.10 |
| ATOM | 2254 | CD | PRO | A | 814 | −17.742 | −6.559 | 38.415 | 1.00 | 23.22 |
| ATOM | 2255 | CA | PRO | A | 814 | −15.916 | −8.003 | 37.741 | 1.00 | 23.12 |
| ATOM | 2256 | CB | PRO | A | 814 | −16.116 | −8.048 | 39.253 | 1.00 | 22.48 |
| ATOM | 2257 | CG | PRO | A | 814 | −16.722 | −6.697 | 39.537 | 1.00 | 22.19 |
| ATOM | 2258 | C | PRO | A | 814 | −15.842 | −9.392 | 37.115 | 1.00 | 23.05 |
| ATOM | 2259 | O | PRO | A | 814 | −14.792 | −9.796 | 36.619 | 1.00 | 23.01 |
| ATOM | 2260 | N | SER | A | 815 | −16.952 | −10.123 | 37.129 | 1.00 | 24.56 |
| ATOM | 2261 | CA | SER | A | 815 | −16.955 | −11.465 | 36.555 | 1.00 | 24.61 |
| ATOM | 2262 | CB | SER | A | 815 | −18.286 | −12.174 | 36.844 | 1.00 | 32.62 |
| ATOM | 2263 | OG | SER | A | 815 | −19.393 | −11.454 | 36.330 | 1.00 | 33.36 |
| ATOM | 2264 | C | SER | A | 815 | −16.687 | −11.440 | 35.049 | 1.00 | 24.80 |
| ATOM | 2265 | O | SER | A | 815 | −16.055 | −12.349 | 34.513 | 1.00 | 25.10 |
| ATOM | 2266 | N | MET | A | 816 | −17.153 | −10.398 | 34.368 | 1.00 | 25.71 |
| ATOM | 2267 | CA | MET | A | 816 | −16.938 | −10.286 | 32.925 | 1.00 | 24.80 |
| ATOM | 2268 | CB | MET | A | 816 | −17.820 | −9.188 | 32.332 | 1.00 | 40.93 |
| ATOM | 2269 | CG | MET | A | 816 | −19.302 | −9.432 | 32.489 | 1.00 | 41.93 |
| ATOM | 2270 | SD | MET | A | 816 | −20.264 | −8.316 | 31.459 | 1.00 | 42.83 |
| ATOM | 2271 | CE | MET | A | 816 | −19.866 | −6.716 | 32.201 | 1.00 | 41.73 |
| ATOM | 2272 | C | MET | A | 816 | −15.479 | −10.000 | 32.585 | 1.00 | 24.22 |
| ATOM | 2273 | O | MET | A | 816 | −14.969 | −10.476 | 31.574 | 1.00 | 24.07 |
| ATOM | 2274 | N | GLN | A | 817 | −14.806 | −9.216 | 33.420 | 1.00 | 24.08 |
| ATOM | 2275 | CA | GLN | A | 817 | −13.403 | −8.909 | 33.182 | 1.00 | 22.68 |
| ATOM | 2276 | CB | GLN | A | 817 | −12.904 | −7.844 | 34.161 | 1.00 | 21.90 |
| ATOM | 2277 | CG | GLN | A | 817 | −13.287 | −6.415 | 33.786 | 1.00 | 21.57 |
| ATOM | 2278 | CD | GLN | A | 817 | −12.797 | −6.020 | 32.407 | 1.00 | 22.28 |
| ATOM | 2279 | OE1 | GLN | A | 817 | −13.389 | −6.392 | 31.396 | 1.00 | 21.33 |
| ATOM | 2280 | NE2 | GLN | A | 817 | −11.704 | −5.272 | 32.359 | 1.00 | 21.19 |
| ATOM | 2281 | C | GLN | A | 817 | −12.587 | −10.185 | 33.344 | 1.00 | 24.08 |
| ATOM | 2282 | O | GLN | A | 817 | −11.655 | −10.435 | 32.588 | 1.00 | 22.00 |
| ATOM | 2283 | N | VAL | A | 818 | −12.947 | −10.990 | 34.340 | 1.00 | 22.79 |
| ATOM | 2284 | CA | VAL | A | 818 | −12.262 | −12.256 | 34.582 | 1.00 | 23.81 |
| ATOM | 2285 | CB | VAL | A | 818 | −12.839 | −12.958 | 35.828 | 1.00 | 26.49 |
| ATOM | 2286 | CG1 | VAL | A | 818 | −12.225 | −14.338 | 35.983 | 1.00 | 26.65 |
| ATOM | 2287 | CG2 | VAL | A | 818 | −12.567 | −12.115 | 37.064 | 1.00 | 26.49 |
| ATOM | 2288 | C | VAL | A | 818 | −12.453 | −13.161 | 33.364 | 1.00 | 24.69 |
| ATOM | 2289 | O | VAL | A | 818 | −11.502 | −13.776 | 32.871 | 1.00 | 23.39 |
| ATOM | 2290 | N | GLY | A | 819 | −13.687 | −13.234 | 32.878 | 1.00 | 25.13 |
| ATOM | 2291 | CA | GLY | A | 819 | −13.971 | −14.061 | 31.716 | 1.00 | 24.79 |
| ATOM | 2292 | C | GLY | A | 819 | −13.221 | −13.571 | 30.490 | 1.00 | 24.86 |
| ATOM | 2293 | O | GLY | A | 819 | −12.729 | −14.354 | 29.677 | 1.00 | 25.43 |
| ATOM | 2294 | N | PHE | A | 820 | −13.140 | −12.255 | 30.355 | 1.00 | 24.15 |
| ATOM | 2295 | CA | PHE | A | 820 | −12.440 | −11.635 | 29.241 | 1.00 | 22.60 |
| ATOM | 2296 | CB | PHE | A | 820 | −12.606 | −10.113 | 29.336 | 1.00 | 22.65 |
| ATOM | 2297 | CG | PHE | A | 820 | −11.736 | −9.335 | 28.389 | 1.00 | 22.10 |
| ATOM | 2298 | CD1 | PHE | A | 820 | −11.863 | −9.490 | 27.011 | 1.00 | 22.63 |
| ATOM | 2299 | CD2 | PHE | A | 820 | −10.803 | −8.424 | 28.879 | 1.00 | 22.66 |
| ATOM | 2300 | CE1 | PHE | A | 820 | −11.069 | −8.743 | 26.133 | 1.00 | 22.28 |
| ATOM | 2301 | CE2 | PHE | A | 820 | −10.008 | −7.677 | 28.011 | 1.00 | 22.73 |
| ATOM | 2302 | CZ | PHE | A | 820 | −10.143 | −7.839 | 26.637 | 1.00 | 22.74 |
| ATOM | 2303 | C | PHE | A | 820 | −10.964 | −12.024 | 29.293 | 1.00 | 23.46 |
| ATOM | 2304 | O | PHE | A | 820 | −10.357 | −12.369 | 28.283 | 1.00 | 21.24 |
| ATOM | 2305 | N | ILE | A | 821 | −10.390 | −11.984 | 30.488 | 1.00 | 21.23 |
| ATOM | 2306 | CA | ILE | A | 821 | −8.990 | −12.323 | 30.646 | 1.00 | 21.89 |
| ATOM | 2307 | CB | ILE | A | 821 | −8.525 | −12.044 | 32.088 | 1.00 | 22.37 |
| ATOM | 2308 | CG2 | ILE | A | 821 | −7.130 | −12.596 | 32.315 | 1.00 | 22.42 |
| ATOM | 2309 | CG1 | ILE | A | 821 | −8.526 | −10.534 | 32.332 | 1.00 | 21.45 |
| ATOM | 2310 | CD1 | ILE | A | 821 | −8.377 | −10.160 | 33.777 | 1.00 | 23.29 |
| ATOM | 2311 | C | ILE | A | 821 | −8.744 | −13.784 | 30.292 | 1.00 | 22.08 |
| ATOM | 2312 | O | ILE | A | 821 | −7.864 | −14.081 | 29.488 | 1.00 | 24.60 |
| ATOM | 2313 | N | ASP | A | 822 | −9.528 | −14.687 | 30.873 | 1.00 | 31.25 |
| ATOM | 2314 | CA | ASP | A | 822 | −9.364 | −16.113 | 30.600 | 1.00 | 32.20 |
| ATOM | 2315 | CB | ASP | A | 822 | −10.379 | −16.950 | 31.390 | 1.00 | 31.68 |
| ATOM | 2316 | CG | ASP | A | 822 | −10.096 | −16.978 | 32.876 | 1.00 | 30.88 |
| ATOM | 2317 | OD1 | ASP | A | 822 | −8.923 | −16.812 | 33.265 | 1.00 | 31.47 |
| ATOM | 2318 | OD2 | ASP | A | 822 | −11.054 | −17.186 | 33.652 | 1.00 | 31.29 |
| ATOM | 2319 | C | ASP | A | 822 | −9.520 | −16.474 | 29.127 | 1.00 | 33.01 |
| ATOM | 2320 | O | ASP | A | 822 | −8.680 | −17.165 | 28.552 | 1.00 | 33.17 |
| ATOM | 2321 | N | ALA | A | 823 | −10.600 | −15.996 | 28.523 | 1.00 | 31.01 |
| ATOM | 2322 | CA | ALA | A | 823 | −10.912 | −16.309 | 27.131 | 1.00 | 30.74 |
| ATOM | 2323 | CB | ALA | A | 823 | −12.402 | −16.092 | 26.894 | 1.00 | 28.33 |
| ATOM | 2324 | C | ALA | A | 823 | −10.123 | −15.608 | 26.030 | 1.00 | 30.37 |
| ATOM | 2325 | O | ALA | A | 823 | −9.919 | −16.179 | 24.959 | 1.00 | 30.71 |
| ATOM | 2326 | N | ILE | A | 824 | −9.671 | −14.385 | 26.281 | 1.00 | 26.40 |
| ATOM | 2327 | CA | ILE | A | 824 | −8.968 | −13.624 | 25.250 | 1.00 | 25.53 |
| ATOM | 2328 | CB | ILE | A | 824 | −9.761 | −12.325 | 24.898 | 1.00 | 28.97 |
| ATOM | 2329 | CG2 | ILE | A | 824 | −8.978 | −11.487 | 23.899 | 1.00 | 29.02 |
| ATOM | 2330 | CG1 | ILE | A | 824 | −11.154 | −12.665 | 24.357 | 1.00 | 29.19 |
| ATOM | 2331 | CD1 | ILE | A | 824 | −11.151 | −13.353 | 23.013 | 1.00 | 29.99 |
| ATOM | 2332 | C | ILE | A | 824 | −7.537 | −13.185 | 25.546 | 1.00 | 27.07 |
| ATOM | 2333 | O | ILE | A | 824 | −6.639 | −13.358 | 24.722 | 1.00 | 26.42 |
| ATOM | 2334 | N | CYS | A | 825 | −7.324 | −12.617 | 26.726 | 1.00 | 24.64 |
| ATOM | 2335 | CA | CYS | A | 825 | −6.020 | −12.070 | 27.075 | 1.00 | 24.29 |
| ATOM | 2336 | CB | CYS | A | 825 | −6.207 | −11.003 | 28.145 | 1.00 | 22.55 |
| ATOM | 2337 | SG | CYS | A | 825 | −7.489 | −9.813 | 27.703 | 1.00 | 21.61 |
| ATOM | 2338 | C | CYS | A | 825 | −4.878 | −12.979 | 27.493 | 1.00 | 23.81 |
| ATOM | 2339 | O | CYS | A | 825 | −3.776 | −12.856 | 26.971 | 1.00 | 23.60 |
| ATOM | 2340 | N | LEU | A | 826 | −5.131 | −13.866 | 28.448 | 1.00 | 25.10 |
| ATOM | 2341 | CA | LEU | A | 826 | −4.098 | −14.761 | 28.955 | 1.00 | 26.70 |
| ATOM | 2342 | CB | LEU | A | 826 | −4.727 | −15.770 | 29.921 | 1.00 | 38.73 |
| ATOM | 2343 | CG | LEU | A | 826 | −4.228 | −15.764 | 31.370 | 1.00 | 39.48 |
| ATOM | 2344 | CD1 | LEU | A | 826 | −4.024 | −14.343 | 31.867 | 1.00 | 38.99 |
| ATOM | 2345 | CD2 | LEU | A | 826 | −5.230 | −16.498 | 32.243 | 1.00 | 39.26 |
| ATOM | 2346 | C | LEU | A | 826 | −3.301 | −15.492 | 27.873 | 1.00 | 25.78 |
| ATOM | 2347 | O | LEU | A | 826 | −2.072 | −15.427 | 27.854 | 1.00 | 26.14 |
| ATOM | 2348 | N | GLN | A | 827 | −3.993 | −16.179 | 26.972 | 1.00 | 26.44 |
| ATOM | 2349 | CA | GLN | A | 827 | −3.308 | −16.919 | 25.920 | 1.00 | 26.16 |
| ATOM | 2350 | CB | GLN | A | 827 | −4.313 | −17.689 | 25.065 | 1.00 | 31.94 |
| ATOM | 2351 | CG | GLN | A | 827 | −5.146 | −18.687 | 25.839 | 1.00 | 32.38 |
| ATOM | 2352 | CD | GLN | A | 827 | −6.176 | −19.374 | 24.967 | 1.00 | 33.15 |
| ATOM | 2353 | OE1 | GLN | A | 827 | −5.844 | −20.233 | 24.146 | 1.00 | 33.30 |
| ATOM | 2354 | NE2 | GLN | A | 827 | −7.437 | −18.989 | 25.130 | 1.00 | 33.01 |
| ATOM | 2355 | C | GLN | A | 827 | −2.483 | −16.005 | 25.034 | 1.00 | 26.24 |
| ATOM | 2356 | O | GLN | A | 827 | −1.394 | −16.372 | 24.593 | 1.00 | 26.27 |
| ATOM | 2357 | N | LEU | A | 828 | −2.995 | −14.811 | 24.761 | 1.00 | 23.62 |
| ATOM | 2358 | CA | LEU | A | 828 | −2.256 | −13.887 | 23.912 | 1.00 | 23.54 |
| ATOM | 2359 | CB | LEU | A | 828 | −3.121 | −12.678 | 23.547 | 1.00 | 27.47 |
| ATOM | 2360 | CG | LEU | A | 828 | −2.393 | −11.621 | 22.705 | 1.00 | 27.18 |
| ATOM | 2361 | CD1 | LEU | A | 828 | −1.769 | −12.267 | 21.474 | 1.00 | 28.21 |
| ATOM | 2362 | CD2 | LEU | A | 828 | −3.361 | −10.534 | 22.293 | 1.00 | 27.24 |
| ATOM | 2363 | C | LEU | A | 828 | −0.948 | −13.398 | 24.525 | 1.00 | 24.07 |
| ATOM | 2364 | O | LEU | A | 828 | 0.089 | −13.389 | 23.863 | 1.00 | 23.90 |
| ATOM | 2365 | N | TYR | A | 829 | −0.984 | −12.979 | 25.785 | 1.00 | 22.58 |
| ATOM | 2366 | CA | TYR | A | 829 | 0.232 | −12.492 | 26.410 | 1.00 | 23.51 |
| ATOM | 2367 | CB | TYR | A | 829 | −0.108 | −11.655 | 27.652 | 1.00 | 22.13 |
| ATOM | 2368 | CG | TYR | A | 829 | −0.797 | −10.352 | 27.273 | 1.00 | 22.65 |
| ATOM | 2369 | CD1 | TYR | A | 829 | −0.133 | −9.395 | 26.504 | 1.00 | 21.70 |
| ATOM | 2370 | CE1 | TYR | A | 829 | −0.778 | −8.224 | 26.084 | 1.00 | 22.06 |
| ATOM | 2371 | CD2 | TYR | A | 829 | −2.126 | −10.110 | 27.622 | 1.00 | 22.96 |
| ATOM | 2372 | CE2 | TYR | A | 829 | −2.782 | −8.939 | 27.202 | 1.00 | 21.69 |
| ATOM | 2373 | CZ | TYR | A | 829 | −2.096 | −8.009 | 26.437 | 1.00 | 22.78 |
| ATOM | 2374 | OH | TYR | A | 829 | −2.717 | −6.850 | 26.023 | 1.00 | 20.96 |
| ATOM | 2375 | C | TYR | A | 829 | 1.171 | −13.653 | 26.725 | 1.00 | 22.44 |
| ATOM | 2376 | O | TYR | A | 829 | 2.378 | −13.469 | 26.803 | 1.00 | 23.40 |
| ATOM | 2377 | N | GLU | A | 830 | 0.615 | −14.851 | 26.877 | 1.00 | 26.33 |
| ATOM | 2378 | CA | GLU | A | 830 | 1.451 | −16.025 | 27.127 | 1.00 | 27.63 |
| ATOM | 2379 | CB | GLU | A | 830 | 0.602 | −17.227 | 27.539 | 1.00 | 34.99 |
| ATOM | 2380 | CG | GLU | A | 830 | 0.034 | −17.110 | 28.941 | 1.00 | 35.86 |
| ATOM | 2381 | CD | GLU | A | 830 | −0.865 | −18.266 | 29.318 | 1.00 | 36.17 |
| ATOM | 2382 | OE1 | GLU | A | 830 | −1.246 | −18.349 | 30.507 | 1.00 | 35.89 |
| ATOM | 2383 | OE2 | GLU | A | 830 | −1.195 | −19.084 | 28.430 | 1.00 | 36.49 |
| ATOM | 2384 | C | GLU | A | 830 | 2.186 | −16.326 | 25.826 | 1.00 | 26.91 |
| ATOM | 2385 | O | GLU | A | 830 | 3.383 | −16.614 | 25.827 | 1.00 | 27.95 |
| ATOM | 2386 | N | ALA | A | 831 | 1.465 | −16.235 | 24.711 | 1.00 | 26.22 |
| ATOM | 2387 | CA | ALA | A | 831 | 2.056 | −16.489 | 23.398 | 1.00 | 26.36 |
| ATOM | 2388 | CB | ALA | A | 831 | 0.965 | −16.533 | 22.328 | 1.00 | 20.71 |
| ATOM | 2389 | C | ALA | A | 831 | 3.091 | −15.425 | 23.044 | 1.00 | 25.92 |
| ATOM | 2390 | O | ALA | A | 831 | 4.108 | −15.725 | 22.429 | 1.00 | 26.14 |
| ATOM | 2391 | N | LEU | A | 832 | 2.833 | −14.180 | 23.432 | 1.00 | 25.86 |
| ATOM | 2392 | CA | LEU | A | 832 | 3.767 | −13.096 | 23.138 | 1.00 | 24.48 |
| ATOM | 2393 | CB | LEU | A | 832 | 3.127 | −11.738 | 23.447 | 1.00 | 23.29 |
| ATOM | 2394 | CG | LEU | A | 832 | 4.036 | −10.513 | 23.333 | 1.00 | 22.75 |
| ATOM | 2395 | CD1 | LEU | A | 832 | 4.597 | −10.387 | 21.926 | 1.00 | 22.85 |
| ATOM | 2396 | CD2 | LEU | A | 832 | 3.237 | −9.268 | 23.701 | 1.00 | 23.24 |
| ATOM | 2397 | C | LEU | A | 832 | 5.056 | −13.238 | 23.936 | 1.00 | 25.65 |
| ATOM | 2398 | O | LEU | A | 832 | 6.140 | −12.902 | 23.454 | 1.00 | 25.17 |
| ATOM | 2399 | N | THR | A | 833 | 4.931 | −13.720 | 25.166 | 1.00 | 26.35 |
| ATOM | 2400 | CA | THR | A | 833 | 6.092 | −13.910 | 26.021 | 1.00 | 27.50 |
| ATOM | 2401 | CB | THR | A | 833 | 5.654 | −14.289 | 27.446 | 1.00 | 28.58 |
| ATOM | 2402 | OG1 | THR | A | 833 | 4.872 | −13.218 | 27.994 | 1.00 | 28.28 |
| ATOM | 2403 | CG2 | THR | A | 833 | 6.868 | −14.531 | 28.339 | 1.00 | 28.51 |
| ATOM | 2404 | C | THR | A | 833 | 6.968 | −15.007 | 25.418 | 1.00 | 28.22 |
| ATOM | 2405 | O | THR | A | 833 | 8.197 | −14.977 | 25.540 | 1.00 | 28.01 |
| ATOM | 2406 | N | HIS | A | 834 | 6.333 | −15.964 | 24.750 | 1.00 | 32.53 |
| ATOM | 2407 | CA | HIS | A | 834 | 7.080 | −17.044 | 24.117 | 1.00 | 33.13 |
| ATOM | 2408 | CB | HIS | A | 834 | 6.131 | −18.053 | 23.473 | 1.00 | 41.03 |
| ATOM | 2409 | CG | HIS | A | 834 | 6.833 | −19.200 | 22.820 | 1.00 | 41.66 |
| ATOM | 2410 | CD2 | HIS | A | 834 | 7.017 | −19.505 | 21.514 | 1.00 | 41.45 |
| ATOM | 2411 | ND1 | HIS | A | 834 | 7.497 | −20.170 | 23.538 | 1.00 | 41.66 |
| ATOM | 2412 | CE1 | HIS | A | 834 | 8.060 | −21.024 | 22.702 | 1.00 | 41.76 |
| ATOM | 2413 | NE2 | HIS | A | 834 | 7.785 | −20.643 | 21.467 | 1.00 | 41.58 |
| ATOM | 2414 | C | HIS | A | 834 | 7.979 | −16.433 | 23.050 | 1.00 | 33.02 |
| ATOM | 2415 | O | HIS | A | 834 | 9.179 | −16.701 | 23.003 | 1.00 | 33.90 |
| ATOM | 2416 | N | VAL | A | 835 | 7.390 | −15.603 | 22.195 | 1.00 | 34.00 |
| ATOM | 2417 | CA | VAL | A | 835 | 8.142 | −14.943 | 21.136 | 1.00 | 33.47 |
| ATOM | 2418 | CB | VAL | A | 835 | 7.235 | −14.027 | 20.282 | 1.00 | 34.13 |
| ATOM | 2419 | CG1 | VAL | A | 835 | 8.082 | −13.226 | 19.304 | 1.00 | 33.51 |
| ATOM | 2420 | CG2 | VAL | A | 835 | 6.213 | −14.863 | 19.533 | 1.00 | 33.76 |
| ATOM | 2421 | C | VAL | A | 835 | 9.278 | −14.105 | 21.705 | 1.00 | 34.04 |
| ATOM | 2422 | O | VAL | A | 835 | 10.373 | −14.076 | 21.147 | 1.00 | 33.58 |
| ATOM | 2423 | N | SER | A | 836 | 9.017 | −13.416 | 22.813 | 1.00 | 31.52 |
| ATOM | 2424 | CA | SER | A | 836 | 10.041 | −12.585 | 23.436 | 1.00 | 32.24 |
| ATOM | 2425 | CB | SER | A | 836 | 10.016 | −11.176 | 22.838 | 1.00 | 33.50 |
| ATOM | 2426 | OG | SER | A | 836 | 11.125 | −10.416 | 23.284 | 1.00 | 33.58 |
| ATOM | 2427 | C | SER | A | 836 | 9.861 | −12.503 | 24.949 | 1.00 | 31.70 |
| ATOM | 2428 | O | SER | A | 836 | 8.937 | −11.856 | 25.447 | 1.00 | 30.73 |
| ATOM | 2429 | N | GLU | A | 837 | 10.758 | −13.168 | 25.668 | 1.00 | 32.13 |
| ATOM | 2430 | CA | GLU | A | 837 | 10.742 | −13.209 | 27.126 | 1.00 | 32.75 |
| ATOM | 2431 | CB | GLU | A | 837 | 12.035 | −13.862 | 27.624 | 1.00 | 50.88 |
| ATOM | 2432 | CG | GLU | A | 837 | 13.295 | −13.194 | 27.082 | 1.00 | 51.44 |
| ATOM | 2433 | CD | GLU | A | 837 | 14.564 | −13.981 | 27.368 | 1.00 | 51.73 |
| ATOM | 2434 | OE1 | GLU | A | 837 | 14.863 | −14.226 | 28.558 | 1.00 | 51.76 |
| ATOM | 2435 | OE2 | GLU | A | 837 | 15.264 | −14.349 | 26.399 | 1.00 | 51.58 |
| ATOM | 2436 | C | GLU | A | 837 | 10.579 | −11.836 | 27.779 | 1.00 | 32.06 |
| ATOM | 2437 | O | GLU | A | 837 | 9.916 | −11.702 | 28.808 | 1.00 | 32.57 |
| ATOM | 2438 | N | ASP | A | 838 | 11.185 | −10.821 | 27.175 | 1.00 | 27.93 |
| ATOM | 2439 | CA | ASP | A | 838 | 11.135 | −9.463 | 27.699 | 1.00 | 28.49 |
| ATOM | 2440 | CB | ASP | A | 838 | 12.085 | −8.574 | 26.896 | 1.00 | 35.78 |
| ATOM | 2441 | CG | ASP | A | 838 | 13.511 | −9.093 | 26.908 | 1.00 | 37.30 |
| ATOM | 2442 | OD1 | ASP | A | 838 | 14.167 | −9.014 | 27.969 | 1.00 | 36.40 |
| ATOM | 2443 | OD2 | ASP | A | 838 | 13.971 | −9.589 | 25.857 | 1.00 | 37.49 |
| ATOM | 2444 | C | ASP | A | 838 | 9.738 | −8.838 | 27.723 | 1.00 | 27.26 |
| ATOM | 2445 | O | ASP | A | 838 | 9.570 | −7.716 | 28.202 | 1.00 | 27.48 |
| ATOM | 2446 | N | CYS | A | 839 | 8.740 | −9.550 | 27.207 | 1.00 | 26.97 |
| ATOM | 2447 | CA | CYS | A | 839 | 7.372 | −9.035 | 27.207 | 1.00 | 26.65 |
| ATOM | 2448 | CB | CYS | A | 839 | 6.668 | −9.360 | 25.882 | 1.00 | 26.64 |
| ATOM | 2449 | SG | CYS | A | 839 | 7.139 | −8.301 | 24.485 | 1.00 | 26.65 |
| ATOM | 2450 | C | CYS | A | 839 | 6.586 | −9.638 | 28.365 | 1.00 | 27.57 |
| ATOM | 2451 | O | CYS | A | 839 | 5.373 | −9.461 | 28.471 | 1.00 | 26.86 |
| ATOM | 2452 | N | PHE | A | 840 | 7.282 | −10.352 | 29.243 | 1.00 | 26.10 |
| ATOM | 2453 | CA | PHE | A | 840 | 6.626 | −10.976 | 30.385 | 1.00 | 26.52 |
| ATOM | 2454 | CB | PHE | A | 840 | 7.653 | −11.679 | 31.281 | 1.00 | 35.43 |
| ATOM | 2455 | CG | PHE | A | 840 | 7.045 | −12.344 | 32.485 | 1.00 | 35.52 |
| ATOM | 2456 | CD1 | PHE | A | 840 | 6.211 | −13.450 | 32.340 | 1.00 | 35.69 |
| ATOM | 2457 | CD2 | PHE | A | 840 | 7.273 | −11.842 | 33.762 | 1.00 | 35.56 |
| ATOM | 2458 | CE1 | PHE | A | 840 | 5.610 | −14.046 | 33.450 | 1.00 | 35.99 |
| ATOM | 2459 | CE2 | PHE | A | 840 | 6.678 | −12.429 | 34.878 | 1.00 | 35.60 |
| ATOM | 2460 | CZ | PHE | A | 840 | 5.843 | −13.533 | 34.722 | 1.00 | 35.62 |
| ATOM | 2461 | C | PHE | A | 840 | 5.810 | −9.991 | 31.227 | 1.00 | 25.53 |
| ATOM | 2462 | O | PHE | A | 840 | 4.726 | −10.332 | 31.698 | 1.00 | 26.75 |
| ATOM | 2463 | N | PRO | A | 841 | 6.311 | −8.757 | 31.422 | 1.00 | 24.00 |
| ATOM | 2464 | CD | PRO | A | 841 | 7.585 | −8.177 | 30.965 | 1.00 | 27.23 |
| ATOM | 2465 | CA | PRO | A | 841 | 5.567 | −7.783 | 32.225 | 1.00 | 23.95 |
| ATOM | 2466 | CB | PRO | A | 841 | 6.345 | −6.491 | 32.002 | 1.00 | 26.33 |
| ATOM | 2467 | CG | PRO | A | 841 | 7.751 | −6.988 | 31.890 | 1.00 | 27.03 |
| ATOM | 2468 | C | PRO | A | 841 | 4.104 | −7.662 | 31.821 | 1.00 | 24.02 |
| ATOM | 2469 | O | PRO | A | 841 | 3.238 | −7.446 | 32.667 | 1.00 | 23.76 |
| ATOM | 2470 | N | LEU | A | 842 | 3.826 | −7.809 | 30.531 | 1.00 | 23.41 |
| ATOM | 2471 | CA | LEU | A | 842 | 2.447 | −7.711 | 30.069 | 1.00 | 22.32 |
| ATOM | 2472 | CB | LEU | A | 842 | 2.400 | −7.647 | 28.540 | 1.00 | 22.25 |
| ATOM | 2473 | CG | LEU | A | 842 | 3.064 | −6.397 | 27.957 | 1.00 | 22.19 |
| ATOM | 2474 | CD1 | LEU | A | 842 | 3.184 | −6.527 | 26.442 | 1.00 | 21.97 |
| ATOM | 2475 | CD2 | LEU | A | 842 | 2.255 | −5.154 | 28.349 | 1.00 | 23.44 |
| ATOM | 2476 | C | LEU | A | 842 | 1.633 | −8.888 | 30.590 | 1.00 | 23.15 |
| ATOM | 2477 | O | LEU | A | 842 | 0.508 | −8.714 | 31.048 | 1.00 | 22.28 |
| ATOM | 2478 | N | LEU | A | 843 | 2.210 | −10.085 | 30.537 | 1.00 | 20.37 |
| ATOM | 2479 | CA | LEU | A | 843 | 1.528 | −11.277 | 31.019 | 1.00 | 22.54 |
| ATOM | 2480 | CB | LEU | A | 843 | 2.369 | −12.517 | 30.716 | 1.00 | 30.62 |
| ATOM | 2481 | CG | LEU | A | 843 | 1.666 | −13.867 | 30.542 | 1.00 | 32.10 |
| ATOM | 2482 | CD1 | LEU | A | 843 | 2.718 | −14.960 | 30.647 | 1.00 | 31.42 |
| ATOM | 2483 | CD2 | LEU | A | 843 | 0.586 | −14.078 | 31.579 | 1.00 | 31.97 |
| ATOM | 2484 | C | LEU | A | 843 | 1.338 | −11.162 | 32.531 | 1.00 | 20.83 |
| ATOM | 2485 | O | LEU | A | 843 | 0.252 | −11.406 | 33.057 | 1.00 | 22.49 |
| ATOM | 2486 | N | ASP | A | 844 | 2.410 | −10.791 | 33.222 | 1.00 | 25.10 |
| ATOM | 2487 | CA | ASP | A | 844 | 2.381 | −10.650 | 34.673 | 1.00 | 25.84 |
| ATOM | 2488 | CB | ASP | A | 844 | 3.756 | −10.204 | 35.177 | 1.00 | 33.41 |
| ATOM | 2489 | CG | ASP | A | 844 | 3.889 | −10.316 | 36.680 | 1.00 | 33.77 |
| ATOM | 2490 | OD1 | ASP | A | 844 | 3.499 | −11.367 | 37.233 | 1.00 | 33.62 |
| ATOM | 2491 | OD2 | ASP | A | 844 | 4.392 | −9.360 | 37.305 | 1.00 | 34.08 |
| ATOM | 2492 | C | ASP | A | 844 | 1.306 | −9.667 | 35.129 | 1.00 | 25.11 |
| ATOM | 2493 | O | ASP | A | 844 | 0.517 | −9.976 | 36.020 | 1.00 | 25.50 |
| ATOM | 2494 | N | GLY | A | 845 | 1.272 | −8.492 | 34.505 | 1.00 | 23.99 |
| ATOM | 2495 | CA | GLY | A | 845 | 0.282 | −7.488 | 34.861 | 1.00 | 24.47 |
| ATOM | 2496 | C | GLY | A | 845 | −1.141 | −7.954 | 34.612 | 1.00 | 24.36 |
| ATOM | 2497 | O | GLY | A | 845 | −2.057 | −7.628 | 35.366 | 1.00 | 23.75 |
| ATOM | 2498 | N | CYS | A | 846 | −1.325 | −8.720 | 33.542 | 1.00 | 23.31 |
| ATOM | 2499 | CA | CYS | A | 846 | −2.631 | −9.252 | 33.188 | 1.00 | 22.27 |
| ATOM | 2500 | CB | CYS | A | 846 | −2.556 | −9.961 | 31.833 | 1.00 | 24.57 |
| ATOM | 2501 | SG | CYS | A | 846 | −4.118 | −10.668 | 31.278 | 1.00 | 23.77 |
| ATOM | 2502 | C | CYS | A | 846 | −3.093 | −10.236 | 34.255 | 1.00 | 23.19 |
| ATOM | 2503 | O | CYS | A | 846 | −4.237 | −10.197 | 34.699 | 1.00 | 23.71 |
| ATOM | 2504 | N | ARG | A | 847 | −2.197 | −11.126 | 34.669 | 1.00 | 25.45 |
| ATOM | 2505 | CA | ARG | A | 847 | −2.554 | −12.103 | 35.681 | 1.00 | 26.33 |
| ATOM | 2506 | CB | ARG | A | 847 | −1.454 | −13.170 | 35.788 | 1.00 | 44.92 |
| ATOM | 2507 | CG | ARG | A | 847 | −1.334 | −13.973 | 34.489 | 1.00 | 45.65 |
| ATOM | 2508 | CD | ARG | A | 847 | −0.520 | −15.257 | 34.590 | 1.00 | 45.89 |
| ATOM | 2509 | NE | ARG | A | 847 | 0.924 | −15.037 | 34.557 | 1.00 | 45.99 |
| ATOM | 2510 | CZ | ARG | A | 847 | 1.805 | −15.950 | 34.155 | 1.00 | 45.73 |
| ATOM | 2511 | NH1 | ARG | A | 847 | 1.392 | −17.142 | 33.743 | 1.00 | 45.92 |
| ATOM | 2512 | NH2 | ARG | A | 847 | 3.102 | −15.678 | 34.178 | 1.00 | 45.71 |
| ATOM | 2513 | C | ARG | A | 847 | −2.839 | −11.434 | 37.021 | 1.00 | 25.65 |
| ATOM | 2514 | O | ARG | A | 847 | −3.673 | −11.909 | 37.794 | 1.00 | 25.84 |
| ATOM | 2515 | N | LYS | A | 848 | −2.172 | −10.319 | 37.291 | 1.00 | 27.18 |
| ATOM | 2516 | CA | LYS | A | 848 | −2.408 | −9.606 | 38.540 | 1.00 | 27.41 |
| ATOM | 2517 | CB | LYS | A | 848 | −1.344 | −8.529 | 38.755 | 1.00 | 29.68 |
| ATOM | 2518 | CG | LYS | A | 848 | 0.011 | −9.082 | 39.165 | 1.00 | 30.06 |
| ATOM | 2519 | CD | LYS | A | 848 | 1.023 | −7.964 | 39.328 | 1.00 | 30.03 |
| ATOM | 2520 | CE | LYS | A | 848 | 2.406 | −8.518 | 39.644 | 1.00 | 30.39 |
| ATOM | 2521 | NZ | LYS | A | 848 | 3.450 | −7.453 | 39.583 | 1.00 | 30.98 |
| ATOM | 2522 | C | LYS | A | 848 | −3.796 | −8.974 | 38.514 | 1.00 | 27.81 |
| ATOM | 2523 | O | LYS | A | 848 | −4.515 | −8.996 | 39.511 | 1.00 | 28.26 |
| ATOM | 2524 | N | ASN | A | 849 | −4.179 | −8.412 | 37.372 | 1.00 | 28.20 |
| ATOM | 2525 | CA | ASN | A | 849 | −5.498 | −7.807 | 37.270 | 1.00 | 26.64 |
| ATOM | 2526 | CB | ASN | A | 849 | −5.640 | −7.028 | 35.961 | 1.00 | 26.08 |
| ATOM | 2527 | CG | ASN | A | 849 | −4.970 | −5.674 | 36.027 | 1.00 | 25.73 |
| ATOM | 2528 | OD1 | ASN | A | 849 | −4.993 | −5.012 | 37.067 | 1.00 | 26.13 |
| ATOM | 2529 | ND2 | ASN | A | 849 | −4.382 | −5.247 | 34.923 | 1.00 | 26.48 |
| ATOM | 2530 | C | ASN | A | 849 | −6.572 | −8.875 | 37.372 | 1.00 | 27.18 |
| ATOM | 2531 | O | ASN | A | 849 | −7.679 | −8.612 | 37.844 | 1.00 | 26.84 |
| ATOM | 2532 | N | ARG | A | 850 | −6.244 | −10.086 | 36.934 | 1.00 | 25.96 |
| ATOM | 2533 | CA | ARG | A | 850 | −7.194 | −11.177 | 37.017 | 1.00 | 26.10 |
| ATOM | 2534 | CB | ARG | A | 850 | −6.640 | −12.431 | 36.338 | 1.00 | 38.03 |
| ATOM | 2535 | CG | ARG | A | 850 | −7.583 | −13.607 | 36.453 | 1.00 | 38.77 |
| ATOM | 2536 | CD | ARG | A | 850 | −6.976 | −14.898 | 35.941 | 1.00 | 38.89 |
| ATOM | 2537 | NE | ARG | A | 850 | −7.798 | −16.037 | 36.336 | 1.00 | 39.62 |
| ATOM | 2538 | CZ | ARG | A | 850 | −9.075 | −16.181 | 36.003 | 1.00 | 39.00 |
| ATOM | 2539 | NH1 | ARG | A | 850 | −9.669 | −15.255 | 35.264 | 1.00 | 40.11 |
| ATOM | 2540 | NH2 | ARG | A | 850 | −9.765 | −17.235 | 36.417 | 1.00 | 38.97 |
| ATOM | 2541 | C | ARG | A | 850 | −7.513 | −11.488 | 38.480 | 1.00 | 25.71 |
| ATOM | 2542 | O | ARG | A | 850 | −8.675 | −11.647 | 38.849 | 1.00 | 26.96 |
| ATOM | 2543 | N | GLN | A | 851 | −6.476 | −11.564 | 39.311 | 1.00 | 29.93 |
| ATOM | 2544 | CA | GLN | A | 851 | −6.659 | −11.860 | 40.728 | 1.00 | 30.30 |
| ATOM | 2545 | CB | GLN | A | 851 | −5.294 | −11.993 | 41.413 | 1.00 | 53.42 |
| ATOM | 2546 | CG | GLN | A | 851 | −4.938 | −10.873 | 42.375 | 1.00 | 54.29 |
| ATOM | 2547 | CD | GLN | A | 851 | −3.479 | −10.910 | 42.794 | 1.00 | 54.54 |
| ATOM | 2548 | OE1 | GLN | A | 851 | −3.071 | −10.204 | 43.717 | 1.00 | 54.48 |
| ATOM | 2549 | NE2 | GLN | A | 851 | −2.681 | −11.724 | 42.106 | 1.00 | 54.48 |
| ATOM | 2550 | C | GLN | A | 851 | −7.493 | −10.765 | 41.381 | 1.00 | 29.94 |
| ATOM | 2551 | O | GLN | A | 851 | −8.377 | −11.042 | 42.189 | 1.00 | 30.04 |
| ATOM | 2552 | N | LYS | A | 852 | −7.213 | −9.518 | 41.017 | 1.00 | 27.54 |
| ATOM | 2553 | CA | LYS | A | 852 | −7.950 | −8.381 | 41.557 | 1.00 | 27.65 |
| ATOM | 2554 | CB | LYS | A | 852 | −7.378 | −7.075 | 40.998 | 1.00 | 40.34 |
| ATOM | 2555 | CG | LYS | A | 852 | −6.005 | −6.705 | 41.527 | 1.00 | 40.79 |
| ATOM | 2556 | CD | LYS | A | 852 | −6.096 | −6.263 | 42.977 | 1.00 | 40.88 |
| ATOM | 2557 | CE | LYS | A | 852 | −4.760 | −5.766 | 43.497 | 1.00 | 40.95 |
| ATOM | 2558 | NZ | LYS | A | 852 | −4.883 | −5.208 | 44.879 | 1.00 | 41.45 |
| ATOM | 2559 | C | LYS | A | 852 | −9.433 | −8.470 | 41.204 | 1.00 | 27.70 |
| ATOM | 2560 | O | LYS | A | 852 | −10.301 | −8.360 | 42.075 | 1.00 | 27.95 |
| ATOM | 2561 | N | TRP | A | 853 | −9.722 | −8.655 | 39.920 | 1.00 | 24.75 |
| ATOM | 2562 | CA | TRP | A | 853 | −11.103 | −8.747 | 39.467 | 1.00 | 25.36 |
| ATOM | 2563 | CB | TRP | A | 853 | −11.150 | −8.857 | 37.940 | 1.00 | 23.18 |
| ATOM | 2564 | CG | TRP | A | 853 | −10.967 | −7.544 | 37.240 | 1.00 | 23.11 |
| ATOM | 2565 | CD2 | TRP | A | 853 | −11.771 | −6.368 | 37.399 | 1.00 | 22.95 |
| ATOM | 2566 | CE2 | TRP | A | 853 | −11.244 | −5.382 | 36.534 | 1.00 | 22.63 |
| ATOM | 2567 | CE3 | TRP | A | 853 | −12.886 | −6.052 | 38.189 | 1.00 | 23.00 |
| ATOM | 2568 | CD1 | TRP | A | 853 | −10.013 | −7.233 | 36.312 | 1.00 | 23.06 |
| ATOM | 2569 | NE1 | TRP | A | 853 | −10.173 | −5.937 | 35.884 | 1.00 | 22.64 |
| ATOM | 2570 | CZ2 | TRP | A | 853 | −11.794 | −4.101 | 36.436 | 1.00 | 22.97 |
| ATOM | 2571 | CZ3 | TRP | A | 853 | −13.433 | −4.778 | 38.092 | 1.00 | 22.86 |
| ATOM | 2572 | CH2 | TRP | A | 853 | −12.886 | −3.818 | 37.220 | 1.00 | 22.99 |
| ATOM | 2573 | C | TRP | A | 853 | −11.816 | −9.931 | 40.101 | 1.00 | 24.85 |
| ATOM | 2574 | O | TRP | A | 853 | −12.976 | −9.824 | 40.505 | 1.00 | 24.94 |
| ATOM | 2575 | N | GLN | A | 854 | −11.107 | −11.051 | 40.202 | 1.00 | 28.90 |
| ATOM | 2576 | CA | GLN | A | 854 | −11.662 | −12.264 | 40.785 | 1.00 | 30.44 |
| ATOM | 2577 | CB | GLN | A | 854 | −10.673 | −13.419 | 40.625 | 1.00 | 37.29 |
| ATOM | 2578 | CG | GLN | A | 854 | −11.183 | −14.749 | 41.152 | 1.00 | 37.88 |
| ATOM | 2579 | CD | GLN | A | 854 | −12.554 | −15.102 | 40.608 | 1.00 | 38.02 |
| ATOM | 2580 | OE1 | GLN | A | 854 | −12.812 | −14.978 | 39.411 | 1.00 | 38.44 |
| ATOM | 2581 | NE2 | GLN | A | 854 | −13.439 | −15.557 | 41.487 | 1.00 | 38.37 |
| ATOM | 2582 | C | GLN | A | 854 | −12.014 | −12.081 | 42.257 | 1.00 | 30.52 |
| ATOM | 2583 | O | GLN | A | 854 | −12.994 | −12.650 | 42.736 | 1.00 | 31.44 |
| ATOM | 2584 | N | ALA | A | 855 | −11.219 | −11.288 | 42.969 | 1.00 | 32.15 |
| ATOM | 2585 | CA | ALA | A | 855 | −11.465 | −11.039 | 44.388 | 1.00 | 32.96 |
| ATOM | 2586 | CB | ALA | A | 855 | −10.282 | −10.300 | 45.003 | 1.00 | 27.28 |
| ATOM | 2587 | C | ALA | A | 855 | −12.747 | −10.231 | 44.572 | 1.00 | 33.38 |
| ATOM | 2588 | O | ALA | A | 855 | −13.473 | −10.411 | 45.551 | 1.00 | 33.79 |
| ATOM | 2589 | N | LEU | A | 856 | −13.020 | −9.338 | 43.627 | 1.00 | 34.25 |
| ATOM | 2590 | CA | LEU | A | 856 | −14.223 | −8.517 | 43.685 | 1.00 | 34.81 |
| ATOM | 2591 | CB | LEU | A | 856 | −14.102 | −7.325 | 42.732 | 1.00 | 29.75 |
| ATOM | 2592 | CG | LEU | A | 856 | −12.988 | −6.310 | 43.001 | 1.00 | 29.61 |
| ATOM | 2593 | CD1 | LEU | A | 856 | −12.998 | −5.251 | 41.926 | 1.00 | 29.93 |
| ATOM | 2594 | CD2 | LEU | A | 856 | −13.180 | −5.679 | 44.373 | 1.00 | 29.63 |
| ATOM | 2595 | C | LEU | A | 856 | −15.455 | −9.339 | 43.315 | 1.00 | 35.51 |
| ATOM | 2596 | O | LEU | A | 856 | −16.560 | −9.063 | 43.786 | 1.00 | 35.20 |
| ATOM | 2597 | N | ALA | A | 857 | −15.258 | −10.358 | 42.484 | 1.00 | 39.71 |
| ATOM | 2598 | CA | ALA | A | 857 | −16.354 | −11.208 | 42.030 | 1.00 | 40.78 |
| ATOM | 2599 | CB | ALA | A | 857 | −16.044 | −11.745 | 40.640 | 1.00 | 32.69 |
| ATOM | 2600 | C | ALA | A | 857 | −16.710 | −12.369 | 42.956 | 1.00 | 41.78 |
| ATOM | 2601 | O | ALA | A | 857 | −17.849 | −12.833 | 42.951 | 1.00 | 41.70 |
| ATOM | 2602 | N | GLU | A | 858 | −15.748 | −12.847 | 43.738 | 1.00 | 51.08 |
| ATOM | 2603 | CA | GLU | A | 858 | −16.005 | −13.967 | 44.639 | 1.00 | 52.63 |
| ATOM | 2604 | CB | GLU | A | 858 | −14.710 | −14.741 | 44.909 | 1.00 | 56.18 |
| ATOM | 2605 | CG | GLU | A | 858 | −13.570 | −13.890 | 45.433 | 1.00 | 56.32 |
| ATOM | 2606 | CD | GLU | A | 858 | −12.341 | −14.710 | 45.778 | 1.00 | 56.60 |
| ATOM | 2607 | OE1 | GLU | A | 858 | −11.844 | −15.446 | 44.898 | 1.00 | 56.57 |
| ATOM | 2608 | OE2 | GLU | A | 858 | −11.871 | −14.614 | 46.930 | 1.00 | 56.45 |
| ATOM | 2609 | C | GLU | A | 858 | −16.635 | −13.536 | 45.960 | 1.00 | 53.20 |
| ATOM | 2610 | O | GLU | A | 858 | −15.986 | −13.709 | 47.014 | 1.00 | 53.41 |
| ATOM | 2611 | OXT | GLU | A | 858 | −17.776 | −13.032 | 45.927 | 1.00 | 56.42 |
| TER | | | | | | | | | | |
| ATOM | 2613 | CB | THR | B | 537 | 25.255 | 66.687 | 12.776 | 1.00 | 47.44 |
| ATOM | 2614 | OG1 | THR | B | 537 | 24.336 | 67.782 | 12.890 | 1.00 | 47.61 |
| ATOM | 2615 | CG2 | THR | B | 537 | 25.471 | 66.356 | 11.307 | 1.00 | 47.41 |
| ATOM | 2616 | C | THR | B | 537 | 24.866 | 65.684 | 15.026 | 1.00 | 50.26 |
| ATOM | 2617 | O | THR | B | 537 | 24.138 | 65.096 | 15.828 | 1.00 | 50.63 |
| ATOM | 2618 | N | THR | B | 537 | 23.266 | 65.220 | 13.181 | 1.00 | 50.23 |
| ATOM | 2619 | CA | THR | B | 537 | 24.695 | 65.462 | 13.530 | 1.00 | 50.15 |
| ATOM | 2620 | N | ARG | B | 538 | 25.822 | 66.534 | 15.395 | 1.00 | 48.27 |
| ATOM | 2621 | CA | ARG | B | 538 | 26.099 | 66.827 | 16.801 | 1.00 | 48.37 |
| ATOM | 2622 | CB | ARG | B | 538 | 24.804 | 67.211 | 17.532 | 1.00 | 55.34 |
| ATOM | 2623 | CG | ARG | B | 538 | 24.941 | 67.437 | 19.040 | 1.00 | 55.68 |
| ATOM | 2624 | CD | ARG | B | 538 | 25.783 | 68.664 | 19.378 | 1.00 | 55.76 |
| ATOM | 2625 | NE | ARG | B | 538 | 25.806 | 68.927 | 20.818 | 1.00 | 55.63 |
| ATOM | 2626 | CZ | ARG | B | 538 | 26.529 | 69.882 | 21.401 | 1.00 | 55.83 |
| ATOM | 2627 | NH1 | ARG | B | 538 | 27.302 | 70.677 | 20.672 | 1.00 | 55.58 |
| ATOM | 2628 | NH2 | ARG | B | 538 | 26.481 | 70.040 | 22.718 | 1.00 | 55.68 |
| ATOM | 2629 | C | ARG | B | 538 | 26.707 | 65.579 | 17.427 | 1.00 | 48.47 |
| ATOM | 2630 | O | ARG | B | 538 | 27.882 | 65.561 | 17.796 | 1.00 | 47.96 |
| ATOM | 2631 | N | GLU | B | 539 | 25.895 | 64.534 | 17.532 | 1.00 | 51.41 |
| ATOM | 2632 | CA | GLU | B | 539 | 26.331 | 63.265 | 18.096 | 1.00 | 50.79 |
| ATOM | 2633 | CB | GLU | B | 539 | 25.162 | 62.280 | 18.093 | 1.00 | 45.27 |
| ATOM | 2634 | CG | GLU | B | 539 | 25.499 | 60.877 | 18.559 | 1.00 | 45.43 |
| ATOM | 2635 | CD | GLU | B | 539 | 24.274 | 59.985 | 18.595 | 1.00 | 45.09 |
| ATOM | 2636 | OE1 | GLU | B | 539 | 23.553 | 59.931 | 17.576 | 1.00 | 45.02 |
| ATOM | 2637 | OE2 | GLU | B | 539 | 24.032 | 59.340 | 19.636 | 1.00 | 45.34 |
| ATOM | 2638 | C | GLU | B | 539 | 27.490 | 62.719 | 17.268 | 1.00 | 49.89 |
| ATOM | 2639 | O | GLU | B | 539 | 28.360 | 62.015 | 17.783 | 1.00 | 51.14 |
| ATOM | 2640 | N | LEU | B | 540 | 27.493 | 63.056 | 15.981 | 1.00 | 35.39 |
| ATOM | 2641 | CA | LEU | B | 540 | 28.537 | 62.617 | 15.066 | 1.00 | 34.61 |
| ATOM | 2642 | CB | LEU | B | 540 | 28.047 | 62.729 | 13.621 | 1.00 | 33.85 |
| ATOM | 2643 | CG | LEU | B | 540 | 29.094 | 62.491 | 12.529 | 1.00 | 33.88 |
| ATOM | 2644 | CD1 | LEU | B | 540 | 29.755 | 61.135 | 12.722 | 1.00 | 33.93 |
| ATOM | 2645 | CD2 | LEU | B | 540 | 28.436 | 62.579 | 11.166 | 1.00 | 34.03 |
| ATOM | 2646 | C | LEU | B | 540 | 29.798 | 63.451 | 15.245 | 1.00 | 33.92 |
| ATOM | 2647 | O | LEU | B | 540 | 30.910 | 62.923 | 15.296 | 1.00 | 33.74 |
| ATOM | 2648 | N | GLN | B | 541 | 29.620 | 64.763 | 15.328 | 1.00 | 38.91 |
| ATOM | 2649 | CA | GLN | B | 541 | 30.747 | 65.664 | 15.511 | 1.00 | 38.96 |
| ATOM | 2650 | CB | GLN | B | 541 | 30.265 | 67.110 | 15.475 | 1.00 | 49.53 |
| ATOM | 2651 | CG | GLN | B | 541 | 29.654 | 67.501 | 14.147 | 1.00 | 49.81 |
| ATOM | 2652 | CD | GLN | B | 541 | 29.118 | 68.910 | 14.156 | 1.00 | 49.94 |
| ATOM | 2653 | OE1 | GLN | B | 541 | 28.185 | 69.225 | 14.896 | 1.00 | 50.34 |
| ATOM | 2654 | NE2 | GLN | B | 541 | 29.707 | 69.774 | 13.336 | 1.00 | 50.07 |
| ATOM | 2655 | C | GLN | B | 541 | 31.389 | 65.356 | 16.850 | 1.00 | 38.55 |
| ATOM | 2656 | O | GLN | B | 541 | 32.613 | 65.343 | 16.980 | 1.00 | 39.00 |
| ATOM | 2657 | N | SER | B | 542 | 30.551 | 65.099 | 17.847 | 1.00 | 32.80 |
| ATOM | 2658 | CA | SER | B | 542 | 31.034 | 64.772 | 19.177 | 1.00 | 32.42 |
| ATOM | 2659 | CB | SER | B | 542 | 29.861 | 64.645 | 20.153 | 1.00 | 41.30 |
| ATOM | 2660 | OG | SER | B | 542 | 29.144 | 65.863 | 20.263 | 1.00 | 42.04 |
| ATOM | 2661 | C | SER | B | 542 | 31.789 | 63.456 | 19.113 | 1.00 | 30.70 |
| ATOM | 2662 | O | SER | B | 542 | 32.848 | 63.305 | 19.714 | 1.00 | 31.49 |
| ATOM | 2663 | N | LEU | B | 543 | 31.233 | 62.505 | 18.370 | 1.00 | 31.05 |
| ATOM | 2664 | CA | LEU | B | 543 | 31.841 | 61.188 | 18.228 | 1.00 | 30.13 |
| ATOM | 2665 | CB | LEU | B | 543 | 30.895 | 60.257 | 17.460 | 1.00 | 31.49 |
| ATOM | 2666 | CG | LEU | B | 543 | 30.924 | 58.757 | 17.783 | 1.00 | 32.02 |
| ATOM | 2667 | CD1 | LEU | B | 543 | 30.315 | 57.991 | 16.615 | 1.00 | 31.51 |
| ATOM | 2668 | CD2 | LEU | B | 543 | 32.336 | 58.278 | 18.036 | 1.00 | 31.28 |
| ATOM | 2669 | C | LEU | B | 543 | 33.175 | 61.259 | 17.492 | 1.00 | 29.52 |
| ATOM | 2670 | O | LEU | B | 543 | 34.183 | 60.737 | 17.963 | 1.00 | 29.71 |
| ATOM | 2671 | N | ALA | B | 544 | 33.175 | 61.911 | 16.335 | 1.00 | 27.41 |
| ATOM | 2672 | CA | ALA | B | 544 | 34.374 | 62.027 | 15.522 | 1.00 | 26.77 |
| ATOM | 2673 | CB | ALA | B | 544 | 34.040 | 62.722 | 14.213 | 1.00 | 32.00 |
| ATOM | 2674 | C | ALA | B | 544 | 35.529 | 62.748 | 16.214 | 1.00 | 27.56 |
| ATOM | 2675 | O | ALA | B | 544 | 36.685 | 62.357 | 16.075 | 1.00 | 27.33 |
| ATOM | 2676 | N | ALA | B | 545 | 35.214 | 63.801 | 16.960 | 1.00 | 29.78 |
| ATOM | 2677 | CA | ALA | B | 545 | 36.241 | 64.580 | 17.642 | 1.00 | 29.66 |
| ATOM | 2678 | CB | ALA | B | 545 | 35.723 | 65.990 | 17.905 | 1.00 | 33.66 |
| ATOM | 2679 | C | ALA | B | 545 | 36.731 | 63.962 | 18.948 | 1.00 | 30.65 |
| ATOM | 2680 | O | ALA | B | 545 | 37.829 | 64.272 | 19.417 | 1.00 | 29.67 |
| ATOM | 2681 | N | ALA | B | 546 | 35.921 | 63.090 | 19.537 | 1.00 | 29.20 |
| ATOM | 2682 | CA | ALA | B | 546 | 36.284 | 62.464 | 20.801 | 1.00 | 30.23 |
| ATOM | 2683 | CB | ALA | B | 546 | 35.090 | 61.705 | 21.364 | 1.00 | 34.60 |
| ATOM | 2684 | C | ALA | B | 546 | 37.493 | 61.538 | 20.713 | 1.00 | 29.49 |
| ATOM | 2685 | O | ALA | B | 546 | 37.779 | 60.959 | 19.664 | 1.00 | 29.95 |
| ATOM | 2686 | N | VAL | B | 547 | 38.219 | 61.423 | 21.820 | 1.00 | 29.24 |
| ATOM | 2687 | CA | VAL | B | 547 | 39.377 | 60.543 | 21.872 | 1.00 | 29.27 |
| ATOM | 2688 | CB | VAL | B | 547 | 40.359 | 60.950 | 22.990 | 1.00 | 28.05 |
| ATOM | 2689 | CG1 | VAL | B | 547 | 41.419 | 59.877 | 23.158 | 1.00 | 27.67 |
| ATOM | 2690 | CG2 | VAL | B | 547 | 41.010 | 62.282 | 22.650 | 1.00 | 28.10 |
| ATOM | 2691 | C | VAL | B | 547 | 38.845 | 59.151 | 22.179 | 1.00 | 29.34 |
| ATOM | 2692 | O | VAL | B | 547 | 38.009 | 58.988 | 23.066 | 1.00 | 30.26 |
| ATOM | 2693 | N | VAL | B | 548 | 39.324 | 58.152 | 21.443 | 1.00 | 28.29 |
| ATOM | 2694 | CA | VAL | B | 548 | 38.879 | 56.778 | 21.653 | 1.00 | 27.44 |
| ATOM | 2695 | CB | VAL | B | 548 | 38.971 | 55.953 | 20.349 | 1.00 | 28.22 |
| ATOM | 2696 | CG1 | VAL | B | 548 | 38.545 | 54.510 | 20.613 | 1.00 | 27.84 |
| ATOM | 2697 | CG2 | VAL | B | 548 | 38.100 | 56.585 | 19.271 | 1.00 | 28.24 |
| ATOM | 2698 | C | VAL | B | 548 | 39.721 | 56.099 | 22.724 | 1.00 | 26.75 |
| ATOM | 2699 | O | VAL | B | 548 | 40.903 | 55.822 | 22.518 | 1.00 | 26.81 |
| ATOM | 2700 | N | PRO | B | 549 | 39.121 | 55.826 | 23.890 | 1.00 | 24.59 |
| ATOM | 2701 | CD | PRO | B | 549 | 37.783 | 56.267 | 24.317 | 1.00 | 25.72 |
| ATOM | 2702 | CA | PRO | B | 549 | 39.830 | 55.172 | 24.995 | 1.00 | 25.08 |
| ATOM | 2703 | CB | PRO | B | 549 | 38.764 | 55.089 | 26.081 | 1.00 | 25.73 |
| ATOM | 2704 | CG | PRO | B | 549 | 37.932 | 56.312 | 25.818 | 1.00 | 26.01 |
| ATOM | 2705 | C | PRO | B | 549 | 40.362 | 53.795 | 24.600 | 1.00 | 26.03 |
| ATOM | 2706 | O | PRO | B | 549 | 39.894 | 53.197 | 23.628 | 1.00 | 24.91 |
| ATOM | 2707 | N | SER | B | 550 | 41.334 | 53.298 | 25.361 | 1.00 | 27.34 |
| ATOM | 2708 | CA | SER | B | 550 | 41.935 | 51.997 | 25.088 | 1.00 | 27.02 |
| ATOM | 2709 | CB | SER | B | 550 | 43.224 | 51.821 | 25.896 | 1.00 | 31.88 |
| ATOM | 2710 | OG | SER | B | 550 | 42.943 | 51.600 | 27.267 | 1.00 | 31.82 |
| ATOM | 2711 | C | SER | B | 550 | 40.975 | 50.873 | 25.440 | 1.00 | 26.64 |
| ATOM | 2712 | O | SER | B | 550 | 40.052 | 51.062 | 26.229 | 1.00 | 26.84 |
| ATOM | 2713 | N | ALA | B | 551 | 41.193 | 49.701 | 24.853 | 1.00 | 26.78 |
| ATOM | 2714 | CA | ALA | B | 551 | 40.336 | 48.558 | 25.135 | 1.00 | 27.11 |
| ATOM | 2715 | CB | ALA | B | 551 | 40.790 | 47.342 | 24.317 | 1.00 | 24.60 |
| ATOM | 2716 | C | ALA | B | 551 | 40.385 | 48.244 | 26.630 | 1.00 | 27.04 |
| ATOM | 2717 | O | ALA | B | 551 | 39.373 | 47.895 | 27.237 | 1.00 | 26.84 |
| ATOM | 2718 | N | GLN | B | 552 | 41.573 | 48.370 | 27.213 | 1.00 | 28.38 |
| ATOM | 2719 | CA | GLN | B | 552 | 41.775 | 48.112 | 28.636 | 1.00 | 29.31 |
| ATOM | 2720 | CB | GLN | B | 552 | 43.233 | 48.374 | 29.014 | 1.00 | 41.60 |
| ATOM | 2721 | CG | GLN | B | 552 | 44.219 | 47.373 | 28.448 | 1.00 | 42.50 |
| ATOM | 2722 | CD | GLN | B | 552 | 45.646 | 47.898 | 28.453 | 1.00 | 42.52 |
| ATOM | 2723 | OE1 | GLN | B | 552 | 46.015 | 48.738 | 27.626 | 1.00 | 42.19 |
| ATOM | 2724 | NE2 | GLN | B | 552 | 46.453 | 47.413 | 29.392 | 1.00 | 42.63 |
| ATOM | 2725 | C | GLN | B | 552 | 40.878 | 49.011 | 29.475 | 1.00 | 28.87 |
| ATOM | 2726 | O | GLN | B | 552 | 40.149 | 48.544 | 30.355 | 1.00 | 28.59 |
| ATOM | 2727 | N | THR | B | 553 | 40.944 | 50.307 | 29.196 | 1.00 | 30.11 |
| ATOM | 2728 | CA | THR | B | 553 | 40.145 | 51.283 | 29.915 | 1.00 | 29.74 |
| ATOM | 2729 | CB | THR | B | 553 | 40.415 | 52.713 | 29.399 | 1.00 | 32.61 |
| ATOM | 2730 | OG1 | THR | B | 553 | 41.795 | 53.047 | 29.598 | 1.00 | 32.65 |
| ATOM | 2731 | CG2 | THR | B | 553 | 39.541 | 53.718 | 30.142 | 1.00 | 32.91 |
| ATOM | 2732 | C | THR | B | 553 | 38.655 | 50.990 | 29.765 | 1.00 | 29.20 |
| ATOM | 2733 | O | THR | B | 553 | 37.919 | 50.996 | 30.747 | 1.00 | 29.52 |
| ATOM | 2734 | N | LEU | B | 554 | 38.225 | 50.723 | 28.534 | 1.00 | 27.01 |
| ATOM | 2735 | CA | LEU | B | 554 | 36.820 | 50.455 | 28.244 | 1.00 | 26.50 |
| ATOM | 2736 | CB | LEU | B | 554 | 36.562 | 50.590 | 26.741 | 1.00 | 28.84 |
| ATOM | 2737 | CG | LEU | B | 554 | 36.851 | 51.973 | 26.146 | 1.00 | 29.16 |
| ATOM | 2738 | CD1 | LEU | B | 554 | 36.766 | 51.908 | 24.630 | 1.00 | 29.33 |
| ATOM | 2739 | CD2 | LEU | B | 554 | 35.862 | 52.992 | 26.699 | 1.00 | 29.62 |
| ATOM | 2740 | C | LEU | B | 554 | 36.324 | 49.098 | 28.733 | 1.00 | 26.37 |
| ATOM | 2741 | O | LEU | B | 554 | 35.117 | 48.879 | 28.832 | 1.00 | 26.37 |
| ATOM | 2742 | N | LYS | B | 555 | 37.255 | 48.192 | 29.026 | 1.00 | 25.28 |
| ATOM | 2743 | CA | LYS | B | 555 | 36.931 | 46.857 | 29.524 | 1.00 | 24.39 |
| ATOM | 2744 | CB | LYS | B | 555 | 36.089 | 46.958 | 30.802 | 1.00 | 35.97 |
| ATOM | 2745 | CG | LYS | B | 555 | 36.740 | 47.722 | 31.945 | 1.00 | 36.48 |
| ATOM | 2746 | CD | LYS | B | 555 | 35.806 | 47.781 | 33.150 | 1.00 | 36.95 |
| ATOM | 2747 | CE | LYS | B | 555 | 36.409 | 48.579 | 34.297 | 1.00 | 36.81 |
| ATOM | 2748 | NZ | LYS | B | 555 | 35.499 | 48.633 | 35.478 | 1.00 | 37.17 |
| ATOM | 2749 | C | LYS | B | 555 | 36.188 | 45.975 | 28.520 | 1.00 | 24.71 |
| ATOM | 2750 | O | LYS | B | 555 | 35.592 | 44.966 | 28.905 | 1.00 | 24.80 |
| ATOM | 2751 | N | ILE | B | 556 | 36.229 | 46.336 | 27.242 | 1.00 | 24.43 |
| ATOM | 2752 | CA | ILE | B | 556 | 35.520 | 45.556 | 26.231 | 1.00 | 24.37 |
| ATOM | 2753 | CB | ILE | B | 556 | 35.436 | 46.312 | 24.895 | 1.00 | 23.67 |
| ATOM | 2754 | CG2 | ILE | B | 556 | 34.651 | 47.601 | 25.078 | 1.00 | 24.41 |
| ATOM | 2755 | CG1 | ILE | B | 556 | 36.838 | 46.637 | 24.384 | 1.00 | 24.28 |
| ATOM | 2756 | CD1 | ILE | B | 556 | 36.847 | 47.138 | 22.956 | 1.00 | 23.53 |
| ATOM | 2757 | C | ILE | B | 556 | 36.095 | 44.167 | 25.966 | 1.00 | 23.99 |
| ATOM | 2758 | O | ILE | B | 556 | 35.499 | 43.380 | 25.235 | 1.00 | 23.82 |
| ATOM | 2759 | N | THR | B | 557 | 37.246 | 43.860 | 26.551 | 1.00 | 24.82 |
| ATOM | 2760 | CA | THR | B | 557 | 37.843 | 42.545 | 26.345 | 1.00 | 25.70 |
| ATOM | 2761 | CB | THR | B | 557 | 39.381 | 42.593 | 26.486 | 1.00 | 34.81 |
| ATOM | 2762 | OG1 | THR | B | 557 | 39.901 | 43.678 | 25.706 | 1.00 | 34.99 |
| ATOM | 2763 | CG2 | THR | B | 557 | 40.001 | 41.289 | 25.991 | 1.00 | 34.84 |
| ATOM | 2764 | C | THR | B | 557 | 37.289 | 41.526 | 27.344 | 1.00 | 25.75 |
| ATOM | 2765 | O | THR | B | 557 | 37.425 | 40.318 | 27.154 | 1.00 | 25.95 |
| ATOM | 2766 | N | ASP | B | 558 | 36.646 | 42.013 | 28.400 | 1.00 | 21.17 |
| ATOM | 2767 | CA | ASP | B | 558 | 36.099 | 41.126 | 29.423 | 1.00 | 20.89 |
| ATOM | 2768 | CB | ASP | B | 558 | 35.952 | 41.873 | 30.750 | 1.00 | 39.26 |
| ATOM | 2769 | CG | ASP | B | 558 | 37.214 | 42.607 | 31.147 | 1.00 | 40.04 |
| ATOM | 2770 | OD1 | ASP | B | 558 | 38.293 | 41.981 | 31.148 | 1.00 | 40.44 |
| ATOM | 2771 | OD2 | ASP | B | 558 | 37.124 | 43.809 | 31.464 | 1.00 | 40.62 |
| ATOM | 2772 | C | ASP | B | 558 | 34.742 | 40.543 | 29.045 | 1.00 | 21.50 |
| ATOM | 2773 | O | ASP | B | 558 | 33.846 | 41.259 | 28.594 | 1.00 | 21.10 |
| ATOM | 2774 | N | PHE | B | 559 | 34.586 | 39.242 | 29.246 | 1.00 | 27.65 |
| ATOM | 2775 | CA | PHE | B | 559 | 33.322 | 38.583 | 28.942 | 1.00 | 28.56 |
| ATOM | 2776 | CB | PHE | B | 559 | 33.474 | 37.067 | 29.076 | 1.00 | 27.72 |
| ATOM | 2777 | CG | PHE | B | 559 | 34.269 | 36.438 | 27.968 | 1.00 | 27.53 |
| ATOM | 2778 | CD1 | PHE | B | 559 | 33.773 | 36.407 | 26.670 | 1.00 | 27.89 |
| ATOM | 2779 | CD2 | PHE | B | 559 | 35.509 | 35.863 | 28.224 | 1.00 | 27.40 |
| ATOM | 2780 | CE1 | PHE | B | 559 | 34.499 | 35.809 | 25.642 | 1.00 | 27.88 |
| ATOM | 2781 | CE2 | PHE | B | 559 | 36.243 | 35.263 | 27.202 | 1.00 | 27.42 |
| ATOM | 2782 | CZ | PHE | B | 559 | 35.738 | 35.236 | 25.911 | 1.00 | 27.16 |
| ATOM | 2783 | C | PHE | B | 559 | 32.214 | 39.072 | 29.872 | 1.00 | 28.90 |
| ATOM | 2784 | O | PHE | B | 559 | 31.030 | 38.983 | 29.545 | 1.00 | 28.49 |
| ATOM | 2785 | N | SER | B | 560 | 32.604 | 39.597 | 31.029 | 1.00 | 29.75 |
| ATOM | 2786 | CA | SER | B | 560 | 31.638 | 40.087 | 32.008 | 1.00 | 30.25 |
| ATOM | 2787 | CB | SER | B | 560 | 32.204 | 39.924 | 33.421 | 1.00 | 41.19 |
| ATOM | 2788 | OG | SER | B | 560 | 33.408 | 40.654 | 33.576 | 1.00 | 41.28 |
| ATOM | 2789 | C | SER | B | 560 | 31.248 | 41.543 | 31.778 | 1.00 | 30.07 |
| ATOM | 2790 | O | SER | B | 560 | 30.571 | 42.149 | 32.609 | 1.00 | 29.90 |
| ATOM | 2791 | N | PHE | B | 561 | 31.674 | 42.098 | 30.648 | 1.00 | 25.27 |
| ATOM | 2792 | CA | PHE | B | 561 | 31.377 | 43.484 | 30.297 | 1.00 | 24.91 |
| ATOM | 2793 | CB | PHE | B | 561 | 31.775 | 43.744 | 28.842 | 1.00 | 26.48 |
| ATOM | 2794 | CG | PHE | B | 561 | 31.458 | 45.135 | 28.357 | 1.00 | 26.42 |
| ATOM | 2795 | CD1 | PHE | B | 561 | 32.349 | 46.181 | 28.564 | 1.00 | 26.47 |
| ATOM | 2796 | CD2 | PHE | B | 561 | 30.267 | 45.392 | 27.679 | 1.00 | 26.27 |
| ATOM | 2797 | CE1 | PHE | B | 561 | 32.061 | 47.468 | 28.100 | 1.00 | 27.33 |
| ATOM | 2798 | CE2 | PHE | B | 561 | 29.970 | 46.676 | 27.210 | 1.00 | 26.55 |
| ATOM | 2799 | CZ | PHE | B | 561 | 30.870 | 47.715 | 27.421 | 1.00 | 26.39 |
| ATOM | 2800 | C | PHE | B | 561 | 29.904 | 43.837 | 30.476 | 1.00 | 24.69 |
| ATOM | 2801 | O | PHE | B | 561 | 29.020 | 43.040 | 30.170 | 1.00 | 25.07 |
| ATOM | 2802 | N | SER | B | 562 | 29.652 | 45.047 | 30.966 | 1.00 | 26.30 |
| ATOM | 2803 | CA | SER | B | 562 | 28.291 | 45.538 | 31.163 | 1.00 | 27.46 |
| ATOM | 2804 | CB | SER | B | 562 | 27.992 | 45.694 | 32.655 | 1.00 | 44.74 |
| ATOM | 2805 | OG | SER | B | 562 | 26.657 | 46.118 | 32.861 | 1.00 | 45.35 |
| ATOM | 2806 | C | SER | B | 562 | 28.188 | 46.895 | 30.476 | 1.00 | 25.80 |
| ATOM | 2807 | O | SER | B | 562 | 29.092 | 47.721 | 30.593 | 1.00 | 25.80 |
| ATOM | 2808 | N | ASP | B | 563 | 27.094 | 47.120 | 29.756 | 1.00 | 22.70 |
| ATOM | 2809 | CA | ASP | B | 563 | 26.896 | 48.380 | 29.050 | 1.00 | 23.18 |
| ATOM | 2810 | CB | ASP | B | 563 | 26.203 | 48.138 | 27.709 | 1.00 | 23.75 |
| ATOM | 2811 | CG | ASP | B | 563 | 24.700 | 47.931 | 27.861 | 1.00 | 23.45 |
| ATOM | 2812 | OD1 | ASP | B | 563 | 24.282 | 46.853 | 28.330 | 1.00 | 23.97 |
| ATOM | 2813 | OD2 | ASP | B | 563 | 23.936 | 48.858 | 27.522 | 1.00 | 24.26 |
| ATOM | 2814 | C | ASP | B | 563 | 26.014 | 49.330 | 29.847 | 1.00 | 22.69 |
| ATOM | 2815 | O | ASP | B | 563 | 25.889 | 50.504 | 29.503 | 1.00 | 23.59 |
| ATOM | 2816 | N | PHE | B | 564 | 25.398 | 48.813 | 30.902 | 1.00 | 25.43 |
| ATOM | 2817 | CA | PHE | B | 564 | 24.471 | 49.599 | 31.708 | 1.00 | 26.75 |
| ATOM | 2818 | CB | PHE | B | 564 | 24.056 | 48.800 | 32.942 | 1.00 | 37.63 |
| ATOM | 2819 | CG | PHE | B | 564 | 22.817 | 49.324 | 33.606 | 1.00 | 38.09 |
| ATOM | 2820 | CD1 | PHE | B | 564 | 21.604 | 49.337 | 32.921 | 1.00 | 38.40 |
| ATOM | 2821 | CD2 | PHE | B | 564 | 22.858 | 49.809 | 34.907 | 1.00 | 38.36 |
| ATOM | 2822 | CE1 | PHE | B | 564 | 20.446 | 49.826 | 33.525 | 1.00 | 38.54 |
| ATOM | 2823 | CE2 | PHE | B | 564 | 21.706 | 50.301 | 35.522 | 1.00 | 38.90 |
| ATOM | 2824 | CZ | PHE | B | 564 | 20.498 | 50.309 | 34.829 | 1.00 | 38.44 |
| ATOM | 2825 | C | PHE | B | 564 | 24.907 | 50.999 | 32.137 | 1.00 | 27.15 |
| ATOM | 2826 | O | PHE | B | 564 | 24.118 | 51.945 | 32.064 | 1.00 | 26.92 |
| ATOM | 2827 | N | GLU | B | 565 | 26.156 | 51.139 | 32.566 | 1.00 | 29.91 |
| ATOM | 2828 | CA | GLU | B | 565 | 26.654 | 52.431 | 33.034 | 1.00 | 30.93 |
| ATOM | 2829 | CB | GLU | B | 565 | 27.762 | 52.219 | 34.069 | 1.00 | 40.08 |
| ATOM | 2830 | CG | GLU | B | 565 | 27.372 | 51.367 | 35.270 | 1.00 | 40.03 |
| ATOM | 2831 | CD | GLU | B | 565 | 27.050 | 49.935 | 34.890 | 1.00 | 40.48 |
| ATOM | 2832 | OE1 | GLU | B | 565 | 27.787 | 49.363 | 34.058 | 1.00 | 40.51 |
| ATOM | 2833 | OE2 | GLU | B | 565 | 26.066 | 49.378 | 35.428 | 1.00 | 40.01 |
| ATOM | 2834 | C | GLU | B | 565 | 27.173 | 53.361 | 31.944 | 1.00 | 30.09 |
| ATOM | 2835 | O | GLU | B | 565 | 27.567 | 54.497 | 32.227 | 1.00 | 30.95 |
| ATOM | 2836 | N | LEU | B | 566 | 27.167 | 52.894 | 30.700 | 1.00 | 27.08 |
| ATOM | 2837 | CA | LEU | B | 566 | 27.671 | 53.692 | 29.594 | 1.00 | 26.42 |
| ATOM | 2838 | CB | LEU | B | 566 | 28.395 | 52.784 | 28.591 | 1.00 | 35.46 |
| ATOM | 2839 | CG | LEU | B | 566 | 29.522 | 51.884 | 29.106 | 1.00 | 36.46 |
| ATOM | 2840 | CD1 | LEU | B | 566 | 30.022 | 50.996 | 27.969 | 1.00 | 35.95 |
| ATOM | 2841 | CD2 | LEU | B | 566 | 30.659 | 52.730 | 29.659 | 1.00 | 36.32 |
| ATOM | 2842 | C | LEU | B | 566 | 26.600 | 54.483 | 28.860 | 1.00 | 25.95 |
| ATOM | 2843 | O | LEU | B | 566 | 25.454 | 54.048 | 28.756 | 1.00 | 24.61 |
| ATOM | 2844 | N | SER | B | 567 | 26.986 | 55.655 | 28.358 | 1.00 | 25.45 |
| ATOM | 2845 | CA | SER | B | 567 | 26.080 | 56.501 | 27.590 | 1.00 | 25.56 |
| ATOM | 2846 | CB | SER | B | 567 | 26.519 | 57.963 | 27.665 | 1.00 | 33.98 |
| ATOM | 2847 | OG | SER | B | 567 | 27.722 | 58.173 | 26.947 | 1.00 | 34.14 |
| ATOM | 2848 | C | SER | B | 567 | 26.155 | 56.027 | 26.140 | 1.00 | 25.50 |
| ATOM | 2849 | O | SER | B | 567 | 27.047 | 55.247 | 25.791 | 1.00 | 23.97 |
| ATOM | 2850 | N | ASP | B | 568 | 25.234 | 56.490 | 25.300 | 1.00 | 26.74 |
| ATOM | 2851 | CA | ASP | B | 568 | 25.240 | 56.101 | 23.892 | 1.00 | 26.93 |
| ATOM | 2852 | CB | ASP | B | 568 | 24.110 | 56.790 | 23.127 | 1.00 | 31.14 |
| ATOM | 2853 | CG | ASP | B | 568 | 22.760 | 56.161 | 23.390 | 1.00 | 31.71 |
| ATOM | 2854 | OD1 | ASP | B | 568 | 21.783 | 56.584 | 22.743 | 1.00 | 32.07 |
| ATOM | 2855 | OD2 | ASP | B | 568 | 22.678 | 55.246 | 24.240 | 1.00 | 31.51 |
| ATOM | 2856 | C | ASP | B | 568 | 26.569 | 56.455 | 23.241 | 1.00 | 26.60 |
| ATOM | 2857 | O | ASP | B | 568 | 27.130 | 55.656 | 22.494 | 1.00 | 26.36 |
| ATOM | 2858 | N | LEU | B | 569 | 27.071 | 57.656 | 23.526 | 1.00 | 26.00 |
| ATOM | 2859 | CA | LEU | B | 569 | 28.341 | 58.091 | 22.964 | 1.00 | 25.02 |
| ATOM | 2860 | CB | LEU | B | 569 | 28.683 | 59.507 | 23.446 | 1.00 | 35.36 |
| ATOM | 2861 | CG | LEU | B | 569 | 30.009 | 60.124 | 22.980 | 1.00 | 35.65 |
| ATOM | 2862 | CD1 | LEU | B | 569 | 30.050 | 60.203 | 21.464 | 1.00 | 36.29 |
| ATOM | 2863 | CD2 | LEU | B | 569 | 30.153 | 61.516 | 23.583 | 1.00 | 35.93 |
| ATOM | 2864 | C | LEU | B | 569 | 29.432 | 57.113 | 23.388 | 1.00 | 24.35 |
| ATOM | 2865 | O | LEU | B | 569 | 30.267 | 56.716 | 22.574 | 1.00 | 25.31 |
| ATOM | 2866 | N | GLU | B | 570 | 29.412 | 56.715 | 24.659 | 1.00 | 23.22 |
| ATOM | 2867 | CA | GLU | B | 570 | 30.406 | 55.784 | 25.173 | 1.00 | 23.38 |
| ATOM | 2868 | CB | GLU | B | 570 | 30.216 | 55.560 | 26.676 | 1.00 | 33.69 |
| ATOM | 2869 | CG | GLU | B | 570 | 30.567 | 56.787 | 27.522 | 1.00 | 34.19 |
| ATOM | 2870 | CD | GLU | B | 570 | 30.543 | 56.509 | 29.017 | 1.00 | 34.09 |
| ATOM | 2871 | OE1 | GLU | B | 570 | 29.467 | 56.160 | 29.548 | 1.00 | 33.38 |
| ATOM | 2872 | OE2 | GLU | B | 570 | 31.605 | 56.642 | 29.665 | 1.00 | 35.27 |
| ATOM | 2873 | C | GLU | B | 570 | 30.351 | 54.458 | 24.423 | 1.00 | 23.12 |
| ATOM | 2874 | O | GLU | B | 570 | 31.397 | 53.899 | 24.085 | 1.00 | 23.04 |
| ATOM | 2875 | N | THR | B | 571 | 29.148 | 53.954 | 24.155 | 1.00 | 23.20 |
| ATOM | 2876 | CA | THR | B | 571 | 29.038 | 52.692 | 23.423 | 1.00 | 23.72 |
| ATOM | 2877 | CB | THR | B | 571 | 27.582 | 52.146 | 23.367 | 1.00 | 22.43 |
| ATOM | 2878 | OG1 | THR | B | 571 | 26.753 | 53.014 | 22.589 | 1.00 | 23.16 |
| ATOM | 2879 | CG2 | THR | B | 571 | 27.010 | 52.013 | 24.772 | 1.00 | 23.40 |
| ATOM | 2880 | C | THR | B | 571 | 29.564 | 52.865 | 22.000 | 1.00 | 22.59 |
| ATOM | 2881 | O | THR | B | 571 | 30.147 | 51.937 | 21.441 | 1.00 | 23.35 |
| ATOM | 2882 | N | ALA | B | 572 | 29.367 | 54.047 | 21.418 | 1.00 | 23.15 |
| ATOM | 2883 | CA | ALA | B | 572 | 29.852 | 54.323 | 20.065 | 1.00 | 23.02 |
| ATOM | 2884 | CB | ALA | B | 572 | 29.330 | 55.671 | 19.575 | 1.00 | 25.21 |
| ATOM | 2885 | C | ALA | B | 572 | 31.381 | 54.315 | 20.061 | 1.00 | 24.49 |
| ATOM | 2886 | O | ALA | B | 572 | 32.007 | 53.797 | 19.133 | 1.00 | 23.72 |
| ATOM | 2887 | N | LEU | B | 573 | 31.984 | 54.906 | 21.092 | 1.00 | 24.96 |
| ATOM | 2888 | CA | LEU | B | 573 | 33.438 | 54.924 | 21.203 | 1.00 | 25.56 |
| ATOM | 2889 | CB | LEU | B | 573 | 33.870 | 55.787 | 22.395 | 1.00 | 25.61 |
| ATOM | 2890 | CG | LEU | B | 573 | 33.684 | 57.294 | 22.204 | 1.00 | 25.96 |
| ATOM | 2891 | CD1 | LEU | B | 573 | 33.998 | 58.025 | 23.505 | 1.00 | 25.46 |
| ATOM | 2892 | CD2 | LEU | B | 573 | 34.581 | 57.784 | 21.076 | 1.00 | 24.77 |
| ATOM | 2893 | C | LEU | B | 573 | 33.961 | 53.490 | 21.363 | 1.00 | 23.87 |
| ATOM | 2894 | O | LEU | B | 573 | 34.960 | 53.116 | 20.744 | 1.00 | 25.16 |
| ATOM | 2895 | N | CYS | B | 574 | 33.291 | 52.697 | 22.195 | 1.00 | 21.17 |
| ATOM | 2896 | CA | CYS | B | 574 | 33.672 | 51.297 | 22.406 | 1.00 | 20.66 |
| ATOM | 2897 | CB | CYS | B | 574 | 32.687 | 50.593 | 23.342 | 1.00 | 22.74 |
| ATOM | 2898 | SG | CYS | B | 574 | 32.882 | 50.908 | 25.120 | 1.00 | 21.69 |
| ATOM | 2899 | C | CYS | B | 574 | 33.665 | 50.555 | 21.072 | 1.00 | 21.23 |
| ATOM | 2900 | O | CYS | B | 574 | 34.536 | 49.727 | 20.801 | 1.00 | 19.77 |
| ATOM | 2901 | N | THR | B | 575 | 32.668 | 50.849 | 20.248 | 1.00 | 21.05 |
| ATOM | 2902 | CA | THR | B | 575 | 32.543 | 50.203 | 18.948 | 1.00 | 20.83 |
| ATOM | 2903 | CB | THR | B | 575 | 31.211 | 50.590 | 18.273 | 1.00 | 21.54 |
| ATOM | 2904 | OG1 | THR | B | 575 | 30.128 | 50.166 | 19.109 | 1.00 | 22.40 |
| ATOM | 2905 | CG2 | THR | B | 575 | 31.067 | 49.917 | 16.912 | 1.00 | 22.61 |
| ATOM | 2906 | C | THR | B | 575 | 33.722 | 50.565 | 18.052 | 1.00 | 21.63 |
| ATOM | 2907 | O | THR | B | 575 | 34.268 | 49.708 | 17.357 | 1.00 | 20.92 |
| ATOM | 2908 | N | ILE | B | 576 | 34.122 | 51.833 | 18.068 | 1.00 | 21.26 |
| ATOM | 2909 | CA | ILE | B | 576 | 35.256 | 52.261 | 17.254 | 1.00 | 21.61 |
| ATOM | 2910 | CB | ILE | B | 576 | 35.510 | 53.786 | 17.365 | 1.00 | 24.97 |
| ATOM | 2911 | CG2 | ILE | B | 576 | 36.817 | 54.147 | 16.660 | 1.00 | 25.32 |
| ATOM | 2912 | CG1 | ILE | B | 576 | 34.333 | 54.558 | 16.757 | 1.00 | 25.33 |
| ATOM | 2913 | CD1 | ILE | B | 576 | 34.503 | 56.083 | 16.761 | 1.00 | 25.78 |
| ATOM | 2914 | C | ILE | B | 576 | 36.506 | 51.521 | 17.717 | 1.00 | 22.18 |
| ATOM | 2915 | O | ILE | B | 576 | 37.344 | 51.139 | 16.900 | 1.00 | 21.36 |
| ATOM | 2916 | N | ARG | B | 577 | 36.619 | 51.318 | 19.028 | 1.00 | 21.36 |
| ATOM | 2917 | CA | ARG | B | 577 | 37.761 | 50.620 | 19.598 | 1.00 | 22.50 |
| ATOM | 2918 | CB | ARG | B | 577 | 37.749 | 50.702 | 21.131 | 1.00 | 20.87 |
| ATOM | 2919 | CG | ARG | B | 577 | 38.879 | 49.924 | 21.796 | 1.00 | 21.90 |
| ATOM | 2920 | CD | ARG | B | 577 | 40.239 | 50.280 | 21.195 | 1.00 | 21.96 |
| ATOM | 2921 | NE | ARG | B | 577 | 40.657 | 51.648 | 21.501 | 1.00 | 21.18 |
| ATOM | 2922 | CZ | ARG | B | 577 | 41.693 | 52.255 | 20.927 | 1.00 | 22.44 |
| ATOM | 2923 | NH1 | ARG | B | 577 | 42.421 | 51.622 | 20.011 | 1.00 | 22.46 |
| ATOM | 2924 | NH2 | ARG | B | 577 | 42.005 | 53.504 | 21.265 | 1.00 | 22.38 |
| ATOM | 2925 | C | ARG | B | 577 | 37.779 | 49.164 | 19.149 | 1.00 | 21.04 |
| ATOM | 2926 | O | ARG | B | 577 | 38.846 | 48.599 | 18.938 | 1.00 | 23.18 |
| ATOM | 2927 | N | MET | B | 578 | 36.600 | 48.560 | 19.000 | 1.00 | 21.91 |
| ATOM | 2928 | CA | MET | B | 578 | 36.507 | 47.172 | 18.548 | 1.00 | 21.13 |
| ATOM | 2929 | CB | MET | B | 578 | 35.054 | 46.705 | 18.554 | 1.00 | 20.87 |
| ATOM | 2930 | CG | MET | B | 578 | 34.503 | 46.404 | 19.925 | 1.00 | 21.65 |
| ATOM | 2931 | SD | MET | B | 578 | 32.749 | 46.041 | 19.841 | 1.00 | 22.25 |
| ATOM | 2932 | CE | MET | B | 578 | 32.205 | 46.673 | 21.420 | 1.00 | 22.99 |
| ATOM | 2933 | C | MET | B | 578 | 37.079 | 47.018 | 17.135 | 1.00 | 21.37 |
| ATOM | 2934 | O | MET | B | 578 | 37.872 | 46.114 | 16.876 | 1.00 | 21.93 |
| ATOM | 2935 | N | PHE | B | 579 | 36.660 | 47.886 | 16.222 | 1.00 | 21.44 |
| ATOM | 2936 | CA | PHE | B | 579 | 37.160 | 47.840 | 14.850 | 1.00 | 20.75 |
| ATOM | 2937 | CB | PHE | B | 579 | 36.496 | 48.908 | 13.979 | 1.00 | 23.89 |
| ATOM | 2938 | CG | PHE | B | 579 | 35.160 | 48.512 | 13.440 | 1.00 | 22.77 |
| ATOM | 2939 | CD1 | PHE | B | 579 | 34.009 | 48.672 | 14.203 | 1.00 | 22.94 |
| ATOM | 2940 | CD2 | PHE | B | 579 | 35.051 | 47.979 | 12.161 | 1.00 | 22.86 |
| ATOM | 2941 | CE1 | PHE | B | 579 | 32.765 | 48.302 | 13.695 | 1.00 | 23.49 |
| ATOM | 2942 | CE2 | PHE | B | 579 | 33.813 | 47.607 | 11.645 | 1.00 | 23.55 |
| ATOM | 2943 | CZ | PHE | B | 579 | 32.668 | 47.769 | 12.415 | 1.00 | 23.73 |
| ATOM | 2944 | C | PHE | B | 579 | 38.660 | 48.101 | 14.832 | 1.00 | 21.82 |
| ATOM | 2945 | O | PHE | B | 579 | 39.411 | 47.442 | 14.113 | 1.00 | 20.97 |
| ATOM | 2946 | N | THR | B | 580 | 39.085 | 49.074 | 15.628 | 1.00 | 23.77 |
| ATOM | 2947 | CA | THR | B | 580 | 40.488 | 49.450 | 15.684 | 1.00 | 23.88 |
| ATOM | 2948 | CB | THR | B | 580 | 40.684 | 50.693 | 16.572 | 1.00 | 24.40 |
| ATOM | 2949 | OG1 | THR | B | 580 | 39.895 | 51.770 | 16.051 | 1.00 | 25.13 |
| ATOM | 2950 | CG2 | THR | B | 580 | 42.153 | 51.109 | 16.596 | 1.00 | 24.68 |
| ATOM | 2951 | C | THR | B | 580 | 41.402 | 48.338 | 16.174 | 1.00 | 24.19 |
| ATOM | 2952 | O | THR | B | 580 | 42.389 | 48.011 | 15.512 | 1.00 | 24.60 |
| ATOM | 2953 | N | ASP | B | 581 | 41.072 | 47.743 | 17.314 | 1.00 | 26.39 |
| ATOM | 2954 | CA | ASP | B | 581 | 41.903 | 46.687 | 17.872 | 1.00 | 27.24 |
| ATOM | 2955 | CB | ASP | B | 581 | 41.635 | 46.554 | 19.369 | 1.00 | 26.21 |
| ATOM | 2956 | CG | ASP | B | 581 | 42.295 | 47.666 | 20.171 | 1.00 | 26.06 |
| ATOM | 2957 | OD1 | ASP | B | 581 | 42.688 | 48.690 | 19.565 | 1.00 | 25.91 |
| ATOM | 2958 | OD2 | ASP | B | 581 | 42.418 | 47.520 | 21.402 | 1.00 | 26.26 |
| ATOM | 2959 | C | ASP | B | 581 | 41.821 | 45.333 | 17.182 | 1.00 | 27.49 |
| ATOM | 2960 | O | ASP | B | 581 | 42.535 | 44.403 | 17.552 | 1.00 | 27.57 |
| ATOM | 2961 | N | LEU | B | 582 | 40.947 | 45.217 | 16.189 | 1.00 | 23.58 |
| ATOM | 2962 | CA | LEU | B | 582 | 40.859 | 43.984 | 15.415 | 1.00 | 24.50 |
| ATOM | 2963 | CB | LEU | B | 582 | 39.395 | 43.600 | 15.139 | 1.00 | 22.05 |
| ATOM | 2964 | CG | LEU | B | 582 | 38.660 | 42.981 | 16.337 | 1.00 | 21.75 |
| ATOM | 2965 | CD1 | LEU | B | 582 | 37.178 | 42.787 | 16.035 | 1.00 | 22.80 |
| ATOM | 2966 | CD2 | LEU | B | 582 | 39.309 | 41.648 | 16.680 | 1.00 | 21.90 |
| ATOM | 2967 | C | LEU | B | 582 | 41.605 | 44.305 | 14.117 | 1.00 | 24.79 |
| ATOM | 2968 | O | LEU | B | 582 | 41.591 | 43.531 | 13.153 | 1.00 | 25.28 |
| ATOM | 2969 | N | ASN | B | 583 | 42.255 | 45.469 | 14.117 | 1.00 | 27.83 |
| ATOM | 2970 | CA | ASN | B | 583 | 43.037 | 45.956 | 12.984 | 1.00 | 30.24 |
| ATOM | 2971 | CB | ASN | B | 583 | 44.235 | 45.037 | 12.740 | 1.00 | 52.59 |
| ATOM | 2972 | CG | ASN | B | 583 | 45.196 | 45.017 | 13.906 | 1.00 | 53.50 |
| ATOM | 2973 | OD1 | ASN | B | 583 | 44.827 | 44.655 | 15.023 | 1.00 | 53.73 |
| ATOM | 2974 | ND2 | ASN | B | 583 | 46.440 | 45.410 | 13.655 | 1.00 | 53.71 |
| ATOM | 2975 | C | ASN | B | 583 | 42.223 | 46.074 | 11.704 | 1.00 | 29.60 |
| ATOM | 2976 | O | ASN | B | 583 | 42.733 | 45.818 | 10.614 | 1.00 | 30.55 |
| ATOM | 2977 | N | LEU | B | 584 | 40.964 | 46.478 | 11.840 | 1.00 | 27.68 |
| ATOM | 2978 | CA | LEU | B | 584 | 40.069 | 46.620 | 10.696 | 1.00 | 27.10 |
| ATOM | 2979 | CB | LEU | B | 584 | 38.632 | 46.323 | 11.131 | 1.00 | 25.59 |
| ATOM | 2980 | CG | LEU | B | 584 | 38.111 | 44.884 | 11.080 | 1.00 | 26.48 |
| ATOM | 2981 | CD1 | LEU | B | 584 | 39.198 | 43.891 | 11.360 | 1.00 | 26.71 |
| ATOM | 2982 | CD2 | LEU | B | 584 | 36.954 | 44.746 | 12.065 | 1.00 | 24.71 |
| ATOM | 2983 | C | LEU | B | 584 | 40.115 | 47.992 | 10.047 | 1.00 | 27.65 |
| ATOM | 2984 | O | LEU | B | 584 | 39.908 | 48.120 | 8.843 | 1.00 | 26.67 |
| ATOM | 2985 | N | VAL | B | 585 | 40.380 | 49.021 | 10.845 | 1.00 | 28.02 |
| ATOM | 2986 | CA | VAL | B | 585 | 40.415 | 50.380 | 10.326 | 1.00 | 28.97 |
| ATOM | 2987 | CB | VAL | B | 585 | 40.586 | 51.400 | 11.467 | 1.00 | 33.37 |
| ATOM | 2988 | CG1 | VAL | B | 585 | 40.639 | 52.812 | 10.902 | 1.00 | 33.53 |
| ATOM | 2989 | CG2 | VAL | B | 585 | 39.436 | 51.267 | 12.448 | 1.00 | 33.91 |
| ATOM | 2990 | C | VAL | B | 585 | 41.502 | 50.620 | 9.283 | 1.00 | 29.73 |
| ATOM | 2991 | O | VAL | B | 585 | 41.210 | 51.056 | 8.172 | 1.00 | 29.62 |
| ATOM | 2992 | N | GLN | B | 586 | 42.749 | 50.330 | 9.638 | 1.00 | 34.73 |
| ATOM | 2993 | CA | GLN | B | 586 | 43.860 | 50.536 | 8.718 | 1.00 | 36.38 |
| ATOM | 2994 | CB | GLN | B | 586 | 45.171 | 50.626 | 9.501 | 1.00 | 56.06 |
| ATOM | 2995 | CG | GLN | B | 586 | 45.261 | 51.887 | 10.349 | 1.00 | 56.60 |
| ATOM | 2996 | CD | GLN | B | 586 | 46.523 | 51.955 | 11.184 | 1.00 | 57.01 |
| ATOM | 2997 | OE1 | GLN | B | 586 | 46.729 | 51.142 | 12.085 | 1.00 | 56.81 |
| ATOM | 2998 | NE2 | GLN | B | 586 | 47.378 | 52.930 | 10.887 | 1.00 | 56.83 |
| ATOM | 2999 | C | GLN | B | 586 | 43.936 | 49.439 | 7.666 | 1.00 | 35.19 |
| ATOM | 3000 | O | GLN | B | 586 | 44.176 | 49.716 | 6.492 | 1.00 | 36.38 |
| ATOM | 3001 | N | ASN | B | 587 | 43.716 | 48.199 | 8.088 | 1.00 | 28.60 |
| ATOM | 3002 | CA | ASN | B | 587 | 43.749 | 47.058 | 7.180 | 1.00 | 27.39 |
| ATOM | 3003 | CB | ASN | B | 587 | 43.208 | 45.816 | 7.893 | 1.00 | 34.50 |
| ATOM | 3004 | CG | ASN | B | 587 | 43.520 | 44.530 | 7.155 | 1.00 | 34.35 |
| ATOM | 3005 | OD1 | ASN | B | 587 | 43.721 | 44.528 | 5.943 | 1.00 | 34.55 |
| ATOM | 3006 | ND2 | ASN | B | 587 | 43.549 | 43.423 | 7.888 | 1.00 | 34.96 |
| ATOM | 3007 | C | ASN | B | 587 | 42.895 | 47.331 | 5.941 | 1.00 | 27.36 |
| ATOM | 3008 | O | ASN | B | 587 | 43.336 | 47.133 | 4.811 | 1.00 | 26.90 |
| ATOM | 3009 | N | PHE | B | 588 | 41.666 | 47.796 | 6.154 | 1.00 | 26.94 |
| ATOM | 3010 | CA | PHE | B | 588 | 40.765 | 48.048 | 5.040 | 1.00 | 26.43 |
| ATOM | 3011 | CB | PHE | B | 588 | 39.423 | 47.350 | 5.317 | 1.00 | 20.94 |
| ATOM | 3012 | CG | PHE | B | 588 | 39.558 | 45.856 | 5.503 | 1.00 | 20.97 |
| ATOM | 3013 | CD1 | PHE | B | 588 | 39.472 | 45.277 | 6.765 | 1.00 | 20.43 |
| ATOM | 3014 | CD2 | PHE | B | 588 | 39.845 | 45.038 | 4.417 | 1.00 | 20.60 |
| ATOM | 3015 | CE1 | PHE | B | 588 | 39.678 | 43.899 | 6.940 | 1.00 | 20.54 |
| ATOM | 3016 | CE2 | PHE | B | 588 | 40.052 | 43.662 | 4.576 | 1.00 | 19.56 |
| ATOM | 3017 | CZ | PHE | B | 588 | 39.969 | 43.091 | 5.842 | 1.00 | 21.12 |
| ATOM | 3018 | C | PHE | B | 588 | 40.573 | 49.521 | 4.670 | 1.00 | 27.33 |
| ATOM | 3019 | O | PHE | B | 588 | 39.625 | 49.878 | 3.973 | 1.00 | 27.04 |
| ATOM | 3020 | N | GLN | B | 589 | 41.502 | 50.365 | 5.111 | 1.00 | 28.79 |
| ATOM | 3021 | CA | GLN | B | 589 | 41.465 | 51.800 | 4.818 | 1.00 | 29.49 |
| ATOM | 3022 | CB | GLN | B | 589 | 41.843 | 52.064 | 3.350 | 1.00 | 45.46 |
| ATOM | 3023 | CG | GLN | B | 589 | 43.334 | 51.986 | 3.014 | 1.00 | 45.82 |
| ATOM | 3024 | CD | GLN | B | 589 | 43.797 | 50.588 | 2.639 | 1.00 | 46.29 |
| ATOM | 3025 | OE1 | GLN | B | 589 | 42.993 | 49.736 | 2.257 | 1.00 | 46.04 |
| ATOM | 3026 | NE2 | GLN | B | 589 | 45.104 | 50.353 | 2.724 | 1.00 | 45.90 |
| ATOM | 3027 | C | GLN | B | 589 | 40.127 | 52.481 | 5.106 | 1.00 | 29.77 |
| ATOM | 3028 | O | GLN | B | 589 | 39.509 | 53.063 | 4.212 | 1.00 | 29.22 |
| ATOM | 3029 | N | MET | B | 590 | 39.686 | 52.421 | 6.357 | 1.00 | 30.86 |
| ATOM | 3030 | CA | MET | B | 590 | 38.432 | 53.050 | 6.743 | 1.00 | 30.28 |
| ATOM | 3031 | CB | MET | B | 590 | 37.759 | 52.259 | 7.865 | 1.00 | 26.11 |
| ATOM | 3032 | CG | MET | B | 590 | 37.254 | 50.888 | 7.485 | 1.00 | 26.11 |
| ATOM | 3033 | SD | MET | B | 590 | 36.366 | 50.192 | 8.891 | 1.00 | 24.81 |
| ATOM | 3034 | CE | MET | B | 590 | 35.005 | 51.232 | 8.953 | 1.00 | 27.02 |
| ATOM | 3035 | C | MET | B | 590 | 38.680 | 54.467 | 7.241 | 1.00 | 30.44 |
| ATOM | 3036 | O | MET | B | 590 | 39.453 | 54.666 | 8.175 | 1.00 | 30.43 |
| ATOM | 3037 | N | LYS | B | 591 | 38.039 | 55.449 | 6.616 | 1.00 | 28.66 |
| ATOM | 3038 | CA | LYS | B | 591 | 38.181 | 56.833 | 7.059 | 1.00 | 29.65 |
| ATOM | 3039 | CB | LYS | B | 591 | 37.556 | 57.798 | 6.045 | 1.00 | 48.54 |
| ATOM | 3040 | CG | LYS | B | 591 | 38.530 | 58.376 | 5.022 | 1.00 | 49.40 |
| ATOM | 3041 | CD | LYS | B | 591 | 39.125 | 57.314 | 4.114 | 1.00 | 49.46 |
| ATOM | 3042 | CE | LYS | B | 591 | 40.123 | 57.925 | 3.141 | 1.00 | 49.63 |
| ATOM | 3043 | NZ | LYS | B | 591 | 39.519 | 59.026 | 2.336 | 1.00 | 49.46 |
| ATOM | 3044 | C | LYS | B | 591 | 37.453 | 56.951 | 8.399 | 1.00 | 28.39 |
| ATOM | 3045 | O | LYS | B | 591 | 36.361 | 56.409 | 8.566 | 1.00 | 28.80 |
| ATOM | 3046 | N | HIS | B | 592 | 38.060 | 57.646 | 9.354 | 1.00 | 26.99 |
| ATOM | 3047 | CA | HIS | B | 592 | 37.447 | 57.803 | 10.669 | 1.00 | 26.60 |
| ATOM | 3048 | CB | HIS | B | 592 | 38.305 | 58.713 | 11.554 | 1.00 | 26.53 |
| ATOM | 3049 | CG | HIS | B | 592 | 37.869 | 58.742 | 12.985 | 1.00 | 26.27 |
| ATOM | 3050 | CD2 | HIS | B | 592 | 37.395 | 59.750 | 13.755 | 1.00 | 26.34 |
| ATOM | 3051 | ND1 | HIS | B | 592 | 37.888 | 57.622 | 13.788 | 1.00 | 26.28 |
| ATOM | 3052 | CE1 | HIS | B | 592 | 37.446 | 57.939 | 14.992 | 1.00 | 26.55 |
| ATOM | 3053 | NE2 | HIS | B | 592 | 37.140 | 59.224 | 14.998 | 1.00 | 26.20 |
| ATOM | 3054 | C | HIS | B | 592 | 36.027 | 58.366 | 10.594 | 1.00 | 26.93 |
| ATOM | 3055 | O | HIS | B | 592 | 35.114 | 57.856 | 11.244 | 1.00 | 26.72 |
| ATOM | 3056 | N | GLU | B | 593 | 35.844 | 59.423 | 9.810 | 1.00 | 25.99 |
| ATOM | 3057 | CA | GLU | B | 593 | 34.532 | 60.049 | 9.675 | 1.00 | 26.68 |
| ATOM | 3058 | CB | GLU | B | 593 | 34.615 | 61.240 | 8.717 | 1.00 | 47.65 |
| ATOM | 3059 | CG | GLU | B | 593 | 35.479 | 62.386 | 9.232 | 1.00 | 48.65 |
| ATOM | 3060 | CD | GLU | B | 593 | 36.880 | 61.942 | 9.628 | 1.00 | 48.66 |
| ATOM | 3061 | OE1 | GLU | B | 593 | 37.595 | 61.373 | 8.773 | 1.00 | 48.66 |
| ATOM | 3062 | OE2 | GLU | B | 593 | 37.266 | 62.160 | 10.798 | 1.00 | 48.65 |
| ATOM | 3063 | C | GLU | B | 593 | 33.479 | 59.056 | 9.188 | 1.00 | 25.92 |
| ATOM | 3064 | O | GLU | B | 593 | 32.368 | 59.011 | 9.717 | 1.00 | 25.44 |
| ATOM | 3065 | N | VAL | B | 594 | 33.837 | 58.257 | 8.188 | 1.00 | 23.11 |
| ATOM | 3066 | CA | VAL | B | 594 | 32.914 | 57.265 | 7.640 | 1.00 | 23.96 |
| ATOM | 3067 | CB | VAL | B | 594 | 33.511 | 56.574 | 6.390 | 1.00 | 21.56 |
| ATOM | 3068 | CG1 | VAL | B | 594 | 32.576 | 55.471 | 5.891 | 1.00 | 21.95 |
| ATOM | 3069 | CG2 | VAL | B | 594 | 33.747 | 57.604 | 5.297 | 1.00 | 21.81 |
| ATOM | 3070 | C | VAL | B | 594 | 32.556 | 56.196 | 8.667 | 1.00 | 23.30 |
| ATOM | 3071 | O | VAL | B | 594 | 31.385 | 55.849 | 8.824 | 1.00 | 23.33 |
| ATOM | 3072 | N | LEU | B | 595 | 33.558 | 55.663 | 9.360 | 1.00 | 24.80 |
| ATOM | 3073 | CA | LEU | B | 595 | 33.300 | 54.639 | 10.364 | 1.00 | 24.45 |
| ATOM | 3074 | CB | LEU | B | 595 | 34.616 | 54.127 | 10.959 | 1.00 | 24.30 |
| ATOM | 3075 | CG | LEU | B | 595 | 34.502 | 53.137 | 12.123 | 1.00 | 25.09 |
| ATOM | 3076 | CD1 | LEU | B | 595 | 33.643 | 51.938 | 11.723 | 1.00 | 23.72 |
| ATOM | 3077 | CD2 | LEU | B | 595 | 35.892 | 52.686 | 12.545 | 1.00 | 23.89 |
| ATOM | 3078 | C | LEU | B | 595 | 32.391 | 55.190 | 11.464 | 1.00 | 24.32 |
| ATOM | 3079 | O | LEU | B | 595 | 31.482 | 54.501 | 11.935 | 1.00 | 24.28 |
| ATOM | 3080 | N | CYS | B | 596 | 32.625 | 56.437 | 11.866 | 1.00 | 23.70 |
| ATOM | 3081 | CA | CYS | B | 596 | 31.794 | 57.057 | 12.895 | 1.00 | 22.77 |
| ATOM | 3082 | CB | CYS | B | 596 | 32.354 | 58.426 | 13.302 | 1.00 | 27.45 |
| ATOM | 3083 | SG | CYS | B | 596 | 33.875 | 58.384 | 14.277 | 1.00 | 27.23 |
| ATOM | 3084 | C | CYS | B | 596 | 30.363 | 57.233 | 12.386 | 1.00 | 22.78 |
| ATOM | 3085 | O | CYS | B | 596 | 29.404 | 56.963 | 13.108 | 1.00 | 22.99 |
| ATOM | 3086 | N | ARG | B | 597 | 30.221 | 57.681 | 11.140 | 1.00 | 22.21 |
| ATOM | 3087 | CA | ARG | B | 597 | 28.899 | 57.893 | 10.562 | 1.00 | 21.97 |
| ATOM | 3088 | CB | ARG | B | 597 | 29.018 | 58.543 | 9.178 | 1.00 | 33.99 |
| ATOM | 3089 | CG | ARG | B | 597 | 27.678 | 58.924 | 8.570 | 1.00 | 35.29 |
| ATOM | 3090 | CD | ARG | B | 597 | 27.807 | 59.645 | 7.228 | 1.00 | 35.28 |
| ATOM | 3091 | NE | ARG | B | 597 | 28.241 | 61.035 | 7.362 | 1.00 | 35.24 |
| ATOM | 3092 | CZ | ARG | B | 597 | 29.494 | 61.455 | 7.216 | 1.00 | 35.37 |
| ATOM | 3093 | NH1 | ARG | B | 597 | 30.463 | 60.596 | 6.926 | 1.00 | 35.79 |
| ATOM | 3094 | NH2 | ARG | B | 597 | 29.778 | 62.745 | 7.354 | 1.00 | 35.68 |
| ATOM | 3095 | C | ARG | B | 597 | 28.149 | 56.563 | 10.459 | 1.00 | 21.87 |
| ATOM | 3096 | O | ARG | B | 597 | 26.954 | 56.496 | 10.729 | 1.00 | 21.94 |
| ATOM | 3097 | N | TRP | B | 598 | 28.862 | 55.511 | 10.069 | 1.00 | 23.25 |
| ATOM | 3098 | CA | TRP | B | 598 | 28.264 | 54.185 | 9.944 | 1.00 | 23.59 |
| ATOM | 3099 | CB | TRP | B | 598 | 29.287 | 53.194 | 9.374 | 1.00 | 21.37 |
| ATOM | 3100 | CG | TRP | B | 598 | 28.744 | 51.811 | 9.234 | 1.00 | 19.95 |
| ATOM | 3101 | CD2 | TRP | B | 598 | 28.905 | 50.725 | 10.157 | 1.00 | 20.76 |
| ATOM | 3102 | CE2 | TRP | B | 598 | 28.159 | 49.636 | 9.653 | 1.00 | 21.16 |
| ATOM | 3103 | CE3 | TRP | B | 598 | 29.602 | 50.568 | 11.363 | 1.00 | 20.58 |
| ATOM | 3104 | CD1 | TRP | B | 598 | 27.937 | 51.349 | 8.237 | 1.00 | 20.72 |
| ATOM | 3105 | NE1 | TRP | B | 598 | 27.581 | 50.044 | 8.481 | 1.00 | 20.01 |
| ATOM | 3106 | CZ2 | TRP | B | 598 | 28.091 | 48.404 | 10.313 | 1.00 | 19.36 |
| ATOM | 3107 | CZ3 | TRP | B | 598 | 29.534 | 49.346 | 12.019 | 1.00 | 21.53 |
| ATOM | 3108 | CH2 | TRP | B | 598 | 28.780 | 48.277 | 11.491 | 1.00 | 21.51 |
| ATOM | 3109 | C | TRP | B | 598 | 27.774 | 53.687 | 11.301 | 1.00 | 23.33 |
| ATOM | 3110 | O | TRP | B | 598 | 26.660 | 53.170 | 11.429 | 1.00 | 22.55 |
| ATOM | 3111 | N | ILE | B | 599 | 28.614 | 53.836 | 12.318 | 1.00 | 19.88 |
| ATOM | 3112 | CA | ILE | B | 599 | 28.250 | 53.401 | 13.652 | 1.00 | 19.76 |
| ATOM | 3113 | CB | ILE | B | 599 | 29.400 | 53.659 | 14.656 | 1.00 | 18.66 |
| ATOM | 3114 | CG2 | ILE | B | 599 | 28.917 | 53.410 | 16.078 | 1.00 | 20.09 |
| ATOM | 3115 | CG1 | ILE | B | 599 | 30.586 | 52.742 | 14.328 | 1.00 | 19.91 |
| ATOM | 3116 | CD1 | ILE | B | 599 | 31.835 | 53.042 | 15.140 | 1.00 | 18.69 |
| ATOM | 3117 | C | ILE | B | 599 | 26.989 | 54.124 | 14.105 | 1.00 | 20.60 |
| ATOM | 3118 | O | ILE | B | 599 | 26.072 | 53.501 | 14.636 | 1.00 | 20.28 |
| ATOM | 3119 | N | LEU | B | 600 | 26.933 | 55.435 | 13.883 | 1.00 | 24.36 |
| ATOM | 3120 | CA | LEU | B | 600 | 25.761 | 56.200 | 14.292 | 1.00 | 24.27 |
| ATOM | 3121 | CB | LEU | B | 600 | 26.023 | 57.700 | 14.135 | 1.00 | 30.88 |
| ATOM | 3122 | CG | LEU | B | 600 | 27.104 | 58.235 | 15.080 | 1.00 | 31.45 |
| ATOM | 3123 | CD1 | LEU | B | 600 | 27.378 | 59.680 | 14.764 | 1.00 | 31.56 |
| ATOM | 3124 | CD2 | LEU | B | 600 | 26.663 | 58.079 | 16.529 | 1.00 | 31.65 |
| ATOM | 3125 | C | LEU | B | 600 | 24.518 | 55.788 | 13.512 | 1.00 | 25.39 |
| ATOM | 3126 | O | LEU | B | 600 | 23.432 | 55.697 | 14.086 | 1.00 | 24.82 |
| ATOM | 3127 | N | SER | B | 601 | 24.681 | 55.522 | 12.216 | 1.00 | 24.46 |
| ATOM | 3128 | CA | SER | B | 601 | 23.563 | 55.098 | 11.373 | 1.00 | 23.76 |
| ATOM | 3129 | CB | SER | B | 601 | 24.018 | 54.931 | 9.920 | 1.00 | 24.34 |
| ATOM | 3130 | OG | SER | B | 601 | 24.232 | 56.181 | 9.287 | 1.00 | 24.61 |
| ATOM | 3131 | C | SER | B | 601 | 23.004 | 53.771 | 11.884 | 1.00 | 23.31 |
| ATOM | 3132 | O | SER | B | 601 | 21.793 | 53.574 | 11.947 | 1.00 | 24.08 |
| ATOM | 3133 | N | VAL | B | 602 | 23.895 | 52.855 | 12.248 | 1.00 | 20.00 |
| ATOM | 3134 | CA | VAL | B | 602 | 23.465 | 51.560 | 12.765 | 1.00 | 19.52 |
| ATOM | 3135 | CB | VAL | B | 602 | 24.681 | 50.622 | 12.998 | 1.00 | 20.56 |
| ATOM | 3136 | CG1 | VAL | B | 602 | 24.271 | 49.399 | 13.806 | 1.00 | 20.50 |
| ATOM | 3137 | CG2 | VAL | B | 602 | 25.258 | 50.193 | 11.659 | 1.00 | 20.79 |
| ATOM | 3138 | C | VAL | B | 602 | 22.685 | 51.742 | 14.073 | 1.00 | 18.74 |
| ATOM | 3139 | O | VAL | B | 602 | 21.598 | 51.188 | 14.243 | 1.00 | 19.51 |
| ATOM | 3140 | N | LYS | B | 603 | 23.230 | 52.533 | 14.989 | 1.00 | 21.59 |
| ATOM | 3141 | CA | LYS | B | 603 | 22.568 | 52.767 | 16.268 | 1.00 | 22.24 |
| ATOM | 3142 | CB | LYS | B | 603 | 23.433 | 53.674 | 17.156 | 1.00 | 32.07 |
| ATOM | 3143 | CG | LYS | B | 603 | 22.812 | 53.968 | 18.523 | 1.00 | 32.67 |
| ATOM | 3144 | CD | LYS | B | 603 | 23.775 | 54.700 | 19.457 | 1.00 | 32.26 |
| ATOM | 3145 | CE | LYS | B | 603 | 24.152 | 56.067 | 18.910 | 1.00 | 33.03 |
| ATOM | 3146 | NZ | LYS | B | 603 | 24.991 | 56.838 | 19.866 | 1.00 | 33.90 |
| ATOM | 3147 | C | LYS | B | 603 | 21.193 | 53.404 | 16.072 | 1.00 | 21.31 |
| ATOM | 3148 | O | LYS | B | 603 | 20.229 | 53.057 | 16.758 | 1.00 | 21.06 |
| ATOM | 3149 | N | LYS | B | 604 | 21.103 | 54.318 | 15.116 | 1.00 | 21.77 |
| ATOM | 3150 | CA | LYS | B | 604 | 19.846 | 55.003 | 14.860 | 1.00 | 22.10 |
| ATOM | 3151 | CB | LYS | B | 604 | 20.071 | 56.179 | 13.904 | 1.00 | 35.05 |
| ATOM | 3152 | CG | LYS | B | 604 | 18.815 | 56.987 | 13.614 | 1.00 | 35.34 |
| ATOM | 3153 | CD | LYS | B | 604 | 19.111 | 58.153 | 12.691 | 1.00 | 35.68 |
| ATOM | 3154 | CE | LYS | B | 604 | 17.841 | 58.897 | 12.313 | 1.00 | 35.70 |
| ATOM | 3155 | NZ | LYS | B | 604 | 18.118 | 60.009 | 11.357 | 1.00 | 35.94 |
| ATOM | 3156 | C | LYS | B | 604 | 18.781 | 54.088 | 14.290 | 1.00 | 23.45 |
| ATOM | 3157 | O | LYS | B | 604 | 17.588 | 54.266 | 14.567 | 1.00 | 23.47 |
| ATOM | 3158 | N | ASN | B | 605 | 19.200 | 53.104 | 13.497 | 1.00 | 24.27 |
| ATOM | 3159 | CA | ASN | B | 605 | 18.238 | 52.198 | 12.887 | 1.00 | 24.84 |
| ATOM | 3160 | CB | ASN | B | 605 | 18.790 | 51.629 | 11.580 | 1.00 | 33.82 |
| ATOM | 3161 | CG | ASN | B | 605 | 18.757 | 52.650 | 10.461 | 1.00 | 34.72 |
| ATOM | 3162 | OD1 | ASN | B | 605 | 19.593 | 53.552 | 10.401 | 1.00 | 35.05 |
| ATOM | 3163 | ND2 | ASN | B | 605 | 17.771 | 52.529 | 9.579 | 1.00 | 34.67 |
| ATOM | 3164 | C | ASN | B | 605 | 17.713 | 51.091 | 13.784 | 1.00 | 25.28 |
| ATOM | 3165 | O | ASN | B | 605 | 17.138 | 50.109 | 13.309 | 1.00 | 26.57 |
| ATOM | 3166 | N | TYR | B | 606 | 17.935 | 51.237 | 15.083 | 1.00 | 22.80 |
| ATOM | 3167 | CA | TYR | B | 606 | 17.381 | 50.301 | 16.047 | 1.00 | 21.47 |
| ATOM | 3168 | CB | TYR | B | 606 | 18.406 | 49.906 | 17.116 | 1.00 | 19.39 |
| ATOM | 3169 | CG | TYR | B | 606 | 19.259 | 48.734 | 16.684 | 1.00 | 18.74 |
| ATOM | 3170 | CD1 | TYR | B | 606 | 20.482 | 48.930 | 16.047 | 1.00 | 19.52 |
| ATOM | 3171 | CE1 | TYR | B | 606 | 21.221 | 47.852 | 15.566 | 1.00 | 19.69 |
| ATOM | 3172 | CD2 | TYR | B | 606 | 18.795 | 47.427 | 16.836 | 1.00 | 18.61 |
| ATOM | 3173 | CE2 | TYR | B | 606 | 19.530 | 46.337 | 16.355 | 1.00 | 19.30 |
| ATOM | 3174 | CZ | TYR | B | 606 | 20.739 | 46.563 | 15.719 | 1.00 | 19.25 |
| ATOM | 3175 | OH | TYR | B | 606 | 21.459 | 45.499 | 15.209 | 1.00 | 18.12 |
| ATOM | 3176 | C | TYR | B | 606 | 16.234 | 51.093 | 16.662 | 1.00 | 22.67 |
| ATOM | 3177 | O | TYR | B | 606 | 16.323 | 52.319 | 16.782 | 1.00 | 24.05 |
| ATOM | 3178 | N | ARG | B | 607 | 15.146 | 50.419 | 17.009 | 1.00 | 23.34 |
| ATOM | 3179 | CA | ARG | B | 607 | 13.997 | 51.106 | 17.602 | 1.00 | 23.02 |
| ATOM | 3180 | CB | ARG | B | 607 | 12.718 | 50.315 | 17.322 | 1.00 | 22.58 |
| ATOM | 3181 | CG | ARG | B | 607 | 12.516 | 50.043 | 15.838 | 1.00 | 22.23 |
| ATOM | 3182 | CD | ARG | B | 607 | 11.149 | 49.467 | 15.533 | 1.00 | 23.26 |
| ATOM | 3183 | NE | ARG | B | 607 | 11.044 | 49.076 | 14.131 | 1.00 | 21.78 |
| ATOM | 3184 | CZ | ARG | B | 607 | 9.916 | 48.698 | 13.537 | 1.00 | 23.35 |
| ATOM | 3185 | NH1 | ARG | B | 607 | 8.780 | 48.661 | 14.220 | 1.00 | 22.77 |
| ATOM | 3186 | NH2 | ARG | B | 607 | 9.931 | 48.344 | 12.258 | 1.00 | 23.68 |
| ATOM | 3187 | C | ARG | B | 607 | 14.247 | 51.243 | 19.100 | 1.00 | 23.25 |
| ATOM | 3188 | O | ARG | B | 607 | 14.123 | 50.282 | 19.855 | 1.00 | 22.89 |
| ATOM | 3189 | N | LYS | B | 608 | 14.602 | 52.450 | 19.526 | 1.00 | 26.06 |
| ATOM | 3190 | CA | LYS | B | 608 | 14.921 | 52.697 | 20.928 | 1.00 | 27.43 |
| ATOM | 3191 | CB | LYS | B | 608 | 15.228 | 54.183 | 21.147 | 1.00 | 43.34 |
| ATOM | 3192 | CG | LYS | B | 608 | 14.041 | 55.104 | 20.946 | 1.00 | 43.63 |
| ATOM | 3193 | CD | LYS | B | 608 | 14.394 | 56.538 | 21.307 | 1.00 | 43.77 |
| ATOM | 3194 | CE | LYS | B | 608 | 13.192 | 57.458 | 21.141 | 1.00 | 44.09 |
| ATOM | 3195 | NZ | LYS | B | 608 | 13.518 | 58.878 | 21.457 | 1.00 | 43.98 |
| ATOM | 3196 | C | LYS | B | 608 | 13.878 | 52.248 | 21.940 | 1.00 | 26.36 |
| ATOM | 3197 | O | LYS | B | 608 | 14.221 | 51.870 | 23.062 | 1.00 | 27.18 |
| ATOM | 3198 | N | ASN | B | 609 | 12.606 | 52.277 | 21.559 | 1.00 | 24.76 |
| ATOM | 3199 | CA | ASN | B | 609 | 11.560 | 51.890 | 22.490 | 1.00 | 25.81 |
| ATOM | 3200 | CB | ASN | B | 609 | 10.305 | 52.718 | 22.222 | 1.00 | 34.37 |
| ATOM | 3201 | CG | ASN | B | 609 | 10.558 | 54.201 | 22.378 | 1.00 | 35.05 |
| ATOM | 3202 | OD1 | ASN | B | 609 | 10.969 | 54.657 | 23.444 | 1.00 | 35.79 |
| ATOM | 3203 | ND2 | ASN | B | 609 | 10.326 | 54.961 | 21.315 | 1.00 | 35.63 |
| ATOM | 3204 | C | ASN | B | 609 | 11.226 | 50.406 | 22.548 | 1.00 | 24.40 |
| ATOM | 3205 | O | ASN | B | 609 | 10.336 | 49.993 | 23.294 | 1.00 | 24.72 |
| ATOM | 3206 | N | VAL | B | 610 | 11.925 | 49.596 | 21.759 | 1.00 | 22.64 |
| ATOM | 3207 | CA | VAL | B | 610 | 11.699 | 48.160 | 21.814 | 1.00 | 21.87 |
| ATOM | 3208 | CB | VAL | B | 610 | 12.205 | 47.456 | 20.536 | 1.00 | 21.44 |
| ATOM | 3209 | CG1 | VAL | B | 610 | 12.237 | 45.953 | 20.746 | 1.00 | 21.17 |
| ATOM | 3210 | CG2 | VAL | B | 610 | 11.276 | 47.783 | 19.377 | 1.00 | 21.07 |
| ATOM | 3211 | C | VAL | B | 610 | 12.513 | 47.729 | 23.037 | 1.00 | 22.60 |
| ATOM | 3212 | O | VAL | B | 610 | 13.704 | 48.016 | 23.127 | 1.00 | 22.78 |
| ATOM | 3213 | N | ALA | B | 611 | 11.865 | 47.051 | 23.976 | 1.00 | 21.43 |
| ATOM | 3214 | CA | ALA | B | 611 | 12.500 | 46.651 | 25.232 | 1.00 | 21.58 |
| ATOM | 3215 | CB | ALA | B | 611 | 11.521 | 45.828 | 26.061 | 1.00 | 24.34 |
| ATOM | 3216 | C | ALA | B | 611 | 13.851 | 45.941 | 25.176 | 1.00 | 21.37 |
| ATOM | 3217 | O | ALA | B | 611 | 14.795 | 46.361 | 25.851 | 1.00 | 21.07 |
| ATOM | 3218 | N | TYR | B | 612 | 13.951 | 44.878 | 24.382 | 1.00 | 20.78 |
| ATOM | 3219 | CA | TYR | B | 612 | 15.202 | 44.119 | 24.301 | 1.00 | 19.11 |
| ATOM | 3220 | CB | TYR | B | 612 | 14.926 | 42.648 | 24.623 | 1.00 | 19.45 |
| ATOM | 3221 | CG | TYR | B | 612 | 16.144 | 41.756 | 24.493 | 1.00 | 19.72 |
| ATOM | 3222 | CD1 | TYR | B | 612 | 16.194 | 40.756 | 23.523 | 1.00 | 19.82 |
| ATOM | 3223 | CE1 | TYR | B | 612 | 17.304 | 39.933 | 23.401 | 1.00 | 19.95 |
| ATOM | 3224 | CD2 | TYR | B | 612 | 17.238 | 41.914 | 25.337 | 1.00 | 19.48 |
| ATOM | 3225 | CE2 | TYR | B | 612 | 18.359 | 41.094 | 25.222 | 1.00 | 20.02 |
| ATOM | 3226 | CZ | TYR | B | 612 | 18.382 | 40.109 | 24.259 | 1.00 | 20.21 |
| ATOM | 3227 | OH | TYR | B | 612 | 19.471 | 39.271 | 24.168 | 1.00 | 20.28 |
| ATOM | 3228 | C | TYR | B | 612 | 15.994 | 44.218 | 22.994 | 1.00 | 20.06 |
| ATOM | 3229 | O | TYR | B | 612 | 17.203 | 44.465 | 23.027 | 1.00 | 19.27 |
| ATOM | 3230 | N | HIS | B | 613 | 15.338 | 44.021 | 21.851 | 1.00 | 20.51 |
| ATOM | 3231 | CA | HIS | B | 613 | 16.046 | 44.096 | 20.569 | 1.00 | 19.40 |
| ATOM | 3232 | CB | HIS | B | 613 | 15.310 | 43.290 | 19.493 | 1.00 | 21.37 |
| ATOM | 3233 | CG | HIS | B | 613 | 15.351 | 41.811 | 19.712 | 1.00 | 21.97 |
| ATOM | 3234 | CD2 | HIS | B | 613 | 16.338 | 40.907 | 19.503 | 1.00 | 21.44 |
| ATOM | 3235 | ND1 | HIS | B | 613 | 14.283 | 41.101 | 20.216 | 1.00 | 21.50 |
| ATOM | 3236 | CE1 | HIS | B | 613 | 14.608 | 39.824 | 20.307 | 1.00 | 22.37 |
| ATOM | 3237 | NE2 | HIS | B | 613 | 15.851 | 39.679 | 19.881 | 1.00 | 22.50 |
| ATOM | 3238 | C | HIS | B | 613 | 16.229 | 45.530 | 20.093 | 1.00 | 19.18 |
| ATOM | 3239 | O | HIS | B | 613 | 15.583 | 45.971 | 19.149 | 1.00 | 19.28 |
| ATOM | 3240 | N | ASN | B | 614 | 17.137 | 46.241 | 20.749 | 1.00 | 19.04 |
| ATOM | 3241 | CA | ASN | B | 614 | 17.429 | 47.631 | 20.425 | 1.00 | 19.60 |
| ATOM | 3242 | CB | ASN | B | 614 | 16.895 | 48.526 | 21.541 | 1.00 | 24.32 |
| ATOM | 3243 | CG | ASN | B | 614 | 17.245 | 47.996 | 22.914 | 1.00 | 24.73 |
| ATOM | 3244 | OD1 | ASN | B | 614 | 18.405 | 47.678 | 23.188 | 1.00 | 26.31 |
| ATOM | 3245 | ND2 | ASN | B | 614 | 16.247 | 47.890 | 23.787 | 1.00 | 24.39 |
| ATOM | 3246 | C | ASN | B | 614 | 18.948 | 47.761 | 20.311 | 1.00 | 19.61 |
| ATOM | 3247 | O | ASN | B | 614 | 19.658 | 46.757 | 20.336 | 1.00 | 19.88 |
| ATOM | 3248 | N | TRP | B | 615 | 19.451 | 48.986 | 20.188 | 1.00 | 20.60 |
| ATOM | 3249 | CA | TRP | B | 615 | 20.893 | 49.189 | 20.052 | 1.00 | 20.16 |
| ATOM | 3250 | CB | TRP | B | 615 | 21.219 | 50.686 | 20.022 | 1.00 | 22.74 |
| ATOM | 3251 | CG | TRP | B | 615 | 22.672 | 50.982 | 20.262 | 1.00 | 23.84 |
| ATOM | 3252 | CD2 | TRP | B | 615 | 23.773 | 50.638 | 19.412 | 1.00 | 23.87 |
| ATOM | 3253 | CE2 | TRP | B | 615 | 24.949 | 51.101 | 20.046 | 1.00 | 24.13 |
| ATOM | 3254 | CE3 | TRP | B | 615 | 23.882 | 49.985 | 18.179 | 1.00 | 24.34 |
| ATOM | 3255 | CD1 | TRP | B | 615 | 23.209 | 51.620 | 21.346 | 1.00 | 23.95 |
| ATOM | 3256 | NE1 | TRP | B | 615 | 24.574 | 51.694 | 21.224 | 1.00 | 24.06 |
| ATOM | 3257 | CZ2 | TRP | B | 615 | 26.222 | 50.932 | 19.485 | 1.00 | 24.74 |
| ATOM | 3258 | CZ3 | TRP | B | 615 | 25.152 | 49.816 | 17.618 | 1.00 | 24.67 |
| ATOM | 3259 | CH2 | TRP | B | 615 | 26.304 | 50.290 | 18.276 | 1.00 | 23.93 |
| ATOM | 3260 | C | TRP | B | 615 | 21.776 | 48.514 | 21.098 | 1.00 | 19.91 |
| ATOM | 3261 | O | TRP | B | 615 | 22.834 | 47.960 | 20.766 | 1.00 | 18.94 |
| ATOM | 3262 | N | ARG | B | 616 | 21.373 | 48.572 | 22.361 | 1.00 | 18.07 |
| ATOM | 3263 | CA | ARG | B | 616 | 22.193 | 47.978 | 23.403 | 1.00 | 19.36 |
| ATOM | 3264 | CB | ARG | B | 616 | 21.610 | 48.286 | 24.785 | 1.00 | 22.36 |
| ATOM | 3265 | CG | ARG | B | 616 | 21.612 | 49.788 | 25.119 | 1.00 | 22.18 |
| ATOM | 3266 | CD | ARG | B | 616 | 23.029 | 50.377 | 25.052 | 1.00 | 22.30 |
| ATOM | 3267 | NE | ARG | B | 616 | 23.086 | 51.816 | 25.313 | 1.00 | 23.22 |
| ATOM | 3268 | CZ | ARG | B | 616 | 23.686 | 52.364 | 26.368 | 1.00 | 23.02 |
| ATOM | 3269 | NH1 | ARG | B | 616 | 24.280 | 51.600 | 27.272 | 1.00 | 22.92 |
| ATOM | 3270 | NH2 | ARG | B | 616 | 23.714 | 53.685 | 26.504 | 1.00 | 23.10 |
| ATOM | 3271 | C | ARG | B | 616 | 22.392 | 46.479 | 23.208 | 1.00 | 18.33 |
| ATOM | 3272 | O | ARG | B | 616 | 23.483 | 45.970 | 23.476 | 1.00 | 18.59 |
| ATOM | 3273 | N | HIS | B | 617 | 21.363 | 45.772 | 22.736 | 1.00 | 20.45 |
| ATOM | 3274 | CA | HIS | B | 617 | 21.495 | 44.333 | 22.512 | 1.00 | 17.68 |
| ATOM | 3275 | CB | HIS | B | 617 | 20.156 | 43.690 | 22.143 | 1.00 | 17.10 |
| ATOM | 3276 | CG | HIS | B | 617 | 20.300 | 42.309 | 21.571 | 1.00 | 16.72 |
| ATOM | 3277 | CD2 | HIS | B | 617 | 20.008 | 41.819 | 20.343 | 1.00 | 19.45 |
| ATOM | 3278 | ND1 | HIS | B | 617 | 20.842 | 41.261 | 22.283 | 1.00 | 17.70 |
| ATOM | 3279 | CE1 | HIS | B | 617 | 20.880 | 40.183 | 21.517 | 1.00 | 16.30 |
| ATOM | 3280 | NE2 | HIS | B | 617 | 20.380 | 40.497 | 20.336 | 1.00 | 14.56 |
| ATOM | 3281 | C | HIS | B | 617 | 22.507 | 44.058 | 21.402 | 1.00 | 18.17 |
| ATOM | 3282 | O | HIS | B | 617 | 23.343 | 43.151 | 21.528 | 1.00 | 17.84 |
| ATOM | 3283 | N | ALA | B | 618 | 22.420 | 44.836 | 20.326 | 1.00 | 18.42 |
| ATOM | 3284 | CA | ALA | B | 618 | 23.329 | 44.707 | 19.189 | 1.00 | 17.70 |
| ATOM | 3285 | CB | ALA | B | 618 | 22.918 | 45.659 | 18.067 | 1.00 | 21.95 |
| ATOM | 3286 | C | ALA | B | 618 | 24.749 | 45.026 | 19.652 | 1.00 | 17.93 |
| ATOM | 3287 | O | ALA | B | 618 | 25.701 | 44.320 | 19.324 | 1.00 | 18.41 |
| ATOM | 3288 | N | PHE | B | 619 | 24.882 | 46.100 | 20.421 | 1.00 | 17.19 |
| ATOM | 3289 | CA | PHE | B | 619 | 26.184 | 46.502 | 20.936 | 1.00 | 17.81 |
| ATOM | 3290 | CB | PHE | B | 619 | 26.040 | 47.811 | 21.726 | 1.00 | 18.98 |
| ATOM | 3291 | CG | PHE | B | 619 | 27.290 | 48.224 | 22.454 | 1.00 | 18.41 |
| ATOM | 3292 | CD1 | PHE | B | 619 | 28.423 | 48.626 | 21.755 | 1.00 | 18.73 |
| ATOM | 3293 | CD2 | PHE | B | 619 | 27.340 | 48.172 | 23.842 | 1.00 | 19.11 |
| ATOM | 3294 | CE1 | PHE | B | 619 | 29.588 | 48.966 | 22.427 | 1.00 | 19.47 |
| ATOM | 3295 | CE2 | PHE | B | 619 | 28.498 | 48.509 | 24.527 | 1.00 | 19.39 |
| ATOM | 3296 | CZ | PHE | B | 619 | 29.627 | 48.907 | 23.816 | 1.00 | 19.32 |
| ATOM | 3297 | C | PHE | B | 619 | 26.791 | 45.407 | 21.818 | 1.00 | 18.52 |
| ATOM | 3298 | O | PHE | B | 619 | 27.979 | 45.091 | 21.695 | 1.00 | 18.60 |
| ATOM | 3299 | N | ASN | B | 620 | 25.983 | 44.819 | 22.699 | 1.00 | 18.74 |
| ATOM | 3300 | CA | ASN | B | 620 | 26.484 | 43.774 | 23.585 | 1.00 | 19.38 |
| ATOM | 3301 | CB | ASN | B | 620 | 25.457 | 43.424 | 24.670 | 1.00 | 22.15 |
| ATOM | 3302 | CG | ASN | B | 620 | 25.289 | 44.538 | 25.691 | 1.00 | 22.79 |
| ATOM | 3303 | OD1 | ASN | B | 620 | 26.218 | 45.288 | 25.941 | 1.00 | 23.65 |
| ATOM | 3304 | ND2 | ASN | B | 620 | 24.110 | 44.632 | 26.292 | 1.00 | 22.68 |
| ATOM | 3305 | C | ASN | B | 620 | 26.857 | 42.524 | 22.806 | 1.00 | 18.58 |
| ATOM | 3306 | O | ASN | B | 620 | 27.787 | 41.809 | 23.178 | 1.00 | 19.87 |
| ATOM | 3307 | N | THR | B | 621 | 26.134 | 42.262 | 21.723 | 1.00 | 19.05 |
| ATOM | 3308 | CA | THR | B | 621 | 26.438 | 41.099 | 20.903 | 1.00 | 18.12 |
| ATOM | 3309 | CB | THR | B | 621 | 25.352 | 40.874 | 19.823 | 1.00 | 17.52 |
| ATOM | 3310 | OG1 | THR | B | 621 | 24.095 | 40.640 | 20.462 | 1.00 | 18.11 |
| ATOM | 3311 | CG2 | THR | B | 621 | 25.694 | 39.657 | 18.953 | 1.00 | 18.01 |
| ATOM | 3312 | C | THR | B | 621 | 27.796 | 41.323 | 20.233 | 1.00 | 18.57 |
| ATOM | 3313 | O | THR | B | 621 | 28.601 | 40.398 | 20.134 | 1.00 | 19.61 |
| ATOM | 3314 | N | ALA | B | 622 | 28.046 | 42.550 | 19.785 | 1.00 | 19.36 |
| ATOM | 3315 | CA | ALA | B | 622 | 29.310 | 42.896 | 19.140 | 1.00 | 18.22 |
| ATOM | 3316 | CB | ALA | B | 622 | 29.221 | 44.288 | 18.511 | 1.00 | 22.38 |
| ATOM | 3317 | C | ALA | B | 622 | 30.452 | 42.843 | 20.152 | 1.00 | 20.92 |
| ATOM | 3318 | O | ALA | B | 622 | 31.555 | 42.407 | 19.829 | 1.00 | 18.90 |
| ATOM | 3319 | N | GLN | B | 623 | 30.192 | 43.285 | 21.381 | 1.00 | 17.97 |
| ATOM | 3320 | CA | GLN | B | 623 | 31.219 | 43.257 | 22.420 | 1.00 | 19.34 |
| ATOM | 3321 | CB | GLN | B | 623 | 30.709 | 43.970 | 23.677 | 1.00 | 20.66 |
| ATOM | 3322 | CG | GLN | B | 623 | 31.720 | 44.081 | 24.823 | 1.00 | 21.04 |
| ATOM | 3323 | CD | GLN | B | 623 | 31.756 | 42.848 | 25.704 | 1.00 | 21.30 |
| ATOM | 3324 | OE1 | GLN | B | 623 | 30.712 | 42.346 | 26.134 | 1.00 | 22.15 |
| ATOM | 3325 | NE2 | GLN | B | 623 | 32.956 | 42.362 | 25.995 | 1.00 | 21.86 |
| ATOM | 3326 | C | GLN | B | 623 | 31.600 | 41.811 | 22.739 | 1.00 | 18.06 |
| ATOM | 3327 | O | GLN | B | 623 | 32.776 | 41.499 | 22.963 | 1.00 | 19.37 |
| ATOM | 3328 | N | CYS | B | 624 | 30.609 | 40.924 | 22.755 | 1.00 | 18.07 |
| ATOM | 3329 | CA | CYS | B | 624 | 30.880 | 39.519 | 23.027 | 1.00 | 17.94 |
| ATOM | 3330 | CB | CYS | B | 624 | 29.585 | 38.724 | 23.171 | 1.00 | 21.24 |
| ATOM | 3331 | SG | CYS | B | 624 | 29.857 | 37.007 | 23.684 | 1.00 | 20.09 |
| ATOM | 3332 | C | CYS | B | 624 | 31.695 | 38.961 | 21.866 | 1.00 | 19.06 |
| ATOM | 3333 | O | CYS | B | 624 | 32.615 | 38.158 | 22.067 | 1.00 | 18.98 |
| ATOM | 3334 | N | MET | B | 625 | 31.371 | 39.400 | 20.653 | 1.00 | 18.53 |
| ATOM | 3335 | CA | MET | B | 625 | 32.102 | 38.937 | 19.470 | 1.00 | 18.30 |
| ATOM | 3336 | CB | MET | B | 625 | 31.491 | 39.521 | 18.195 | 1.00 | 19.41 |
| ATOM | 3337 | CG | MET | B | 625 | 32.164 | 39.070 | 16.897 | 1.00 | 19.09 |
| ATOM | 3338 | SD | MET | B | 625 | 32.084 | 37.274 | 16.604 | 1.00 | 16.39 |
| ATOM | 3339 | CE | MET | B | 625 | 30.324 | 37.033 | 16.399 | 1.00 | 18.22 |
| ATOM | 3340 | C | MET | B | 625 | 33.555 | 39.393 | 19.606 | 1.00 | 18.55 |
| ATOM | 3341 | O | MET | B | 625 | 34.483 | 38.640 | 19.332 | 1.00 | 19.67 |
| ATOM | 3342 | N | PHE | B | 626 | 33.749 | 40.636 | 20.036 | 1.00 | 19.00 |
| ATOM | 3343 | CA | PHE | B | 626 | 35.101 | 41.156 | 20.225 | 1.00 | 18.99 |
| ATOM | 3344 | CB | PHE | B | 626 | 35.063 | 42.625 | 20.649 | 1.00 | 19.58 |
| ATOM | 3345 | CG | PHE | B | 626 | 36.421 | 43.211 | 20.909 | 1.00 | 19.51 |
| ATOM | 3346 | CD1 | PHE | B | 626 | 36.965 | 43.200 | 22.190 | 1.00 | 21.50 |
| ATOM | 3347 | CD2 | PHE | B | 626 | 37.162 | 43.759 | 19.868 | 1.00 | 20.23 |
| ATOM | 3348 | CE1 | PHE | B | 626 | 38.234 | 43.731 | 22.433 | 1.00 | 19.73 |
| ATOM | 3349 | CE2 | PHE | B | 626 | 38.433 | 44.292 | 20.095 | 1.00 | 20.70 |
| ATOM | 3350 | CZ | PHE | B | 626 | 38.971 | 44.277 | 21.384 | 1.00 | 20.75 |
| ATOM | 3351 | C | PHE | B | 626 | 35.851 | 40.352 | 21.279 | 1.00 | 20.36 |
| ATOM | 3352 | O | PHE | B | 626 | 36.993 | 39.951 | 21.066 | 1.00 | 19.87 |
| ATOM | 3353 | N | ALA | B | 627 | 35.211 | 40.117 | 22.420 | 1.00 | 20.43 |
| ATOM | 3354 | CA | ALA | B | 627 | 35.853 | 39.367 | 23.490 | 1.00 | 21.24 |
| ATOM | 3355 | CB | ALA | B | 627 | 34.950 | 39.322 | 24.713 | 1.00 | 23.11 |
| ATOM | 3356 | C | ALA | B | 627 | 36.192 | 37.949 | 23.033 | 1.00 | 20.95 |
| ATOM | 3357 | O | ALA | B | 627 | 37.274 | 37.433 | 23.329 | 1.00 | 21.81 |
| ATOM | 3358 | N | ALA | B | 628 | 35.267 | 37.327 | 22.305 | 1.00 | 19.64 |
| ATOM | 3359 | CA | ALA | B | 628 | 35.474 | 35.970 | 21.808 | 1.00 | 18.50 |
| ATOM | 3360 | CB | ALA | B | 628 | 34.194 | 35.453 | 21.156 | 1.00 | 21.27 |
| ATOM | 3361 | C | ALA | B | 628 | 36.619 | 35.901 | 20.808 | 1.00 | 18.67 |
| ATOM | 3362 | O | ALA | B | 628 | 37.415 | 34.958 | 20.827 | 1.00 | 21.28 |
| ATOM | 3363 | N | LEU | B | 629 | 36.695 | 36.890 | 19.921 | 1.00 | 19.77 |
| ATOM | 3364 | CA | LEU | B | 629 | 37.748 | 36.926 | 18.911 | 1.00 | 20.08 |
| ATOM | 3365 | CB | LEU | B | 629 | 37.449 | 38.026 | 17.885 | 1.00 | 20.64 |
| ATOM | 3366 | CG | LEU | B | 629 | 36.265 | 37.740 | 16.957 | 1.00 | 20.56 |
| ATOM | 3367 | CD1 | LEU | B | 629 | 35.933 | 38.980 | 16.138 | 1.00 | 20.95 |
| ATOM | 3368 | CD2 | LEU | B | 629 | 36.612 | 36.556 | 16.048 | 1.00 | 21.88 |
| ATOM | 3369 | C | LEU | B | 629 | 39.097 | 37.175 | 19.565 | 1.00 | 20.23 |
| ATOM | 3370 | O | LEU | B | 629 | 40.083 | 36.505 | 19.254 | 1.00 | 20.58 |
| ATOM | 3371 | N | LYS | B | 630 | 39.131 | 38.142 | 20.478 | 1.00 | 22.49 |
| ATOM | 3372 | CA | LYS | B | 630 | 40.354 | 38.517 | 21.184 | 1.00 | 24.06 |
| ATOM | 3373 | CB | LYS | B | 630 | 40.218 | 39.936 | 21.730 | 1.00 | 29.47 |
| ATOM | 3374 | CG | LYS | B | 630 | 40.467 | 41.003 | 20.712 | 1.00 | 30.15 |
| ATOM | 3375 | CD | LYS | B | 630 | 41.918 | 40.980 | 20.304 | 1.00 | 30.05 |
| ATOM | 3376 | CE | LYS | B | 630 | 42.193 | 42.037 | 19.271 | 1.00 | 29.88 |
| ATOM | 3377 | NZ | LYS | B | 630 | 43.597 | 41.963 | 18.828 | 1.00 | 29.81 |
| ATOM | 3378 | C | LYS | B | 630 | 40.727 | 37.597 | 22.331 | 1.00 | 24.07 |
| ATOM | 3379 | O | LYS | B | 630 | 41.585 | 36.724 | 22.197 | 1.00 | 23.83 |
| ATOM | 3380 | N | ALA | B | 631 | 40.091 | 37.815 | 23.475 | 1.00 | 24.13 |
| ATOM | 3381 | CA | ALA | B | 631 | 40.368 | 37.020 | 24.659 | 1.00 | 24.70 |
| ATOM | 3382 | CB | ALA | B | 631 | 39.527 | 37.516 | 25.828 | 1.00 | 25.17 |
| ATOM | 3383 | C | ALA | B | 631 | 40.121 | 35.534 | 24.437 | 1.00 | 25.41 |
| ATOM | 3384 | O | ALA | B | 631 | 40.882 | 34.700 | 24.922 | 1.00 | 25.73 |
| ATOM | 3385 | N | GLY | B | 632 | 39.060 | 35.203 | 23.705 | 1.00 | 24.21 |
| ATOM | 3386 | CA | GLY | B | 632 | 38.743 | 33.804 | 23.468 | 1.00 | 23.42 |
| ATOM | 3387 | C | GLY | B | 632 | 39.586 | 33.166 | 22.380 | 1.00 | 22.70 |
| ATOM | 3388 | O | GLY | B | 632 | 39.440 | 31.978 | 22.090 | 1.00 | 24.46 |
| ATOM | 3389 | N | LYS | B | 633 | 40.456 | 33.968 | 21.775 | 1.00 | 23.53 |
| ATOM | 3390 | CA | LYS | B | 633 | 41.357 | 33.531 | 20.717 | 1.00 | 24.70 |
| ATOM | 3391 | CB | LYS | B | 633 | 42.419 | 32.592 | 21.292 | 1.00 | 32.80 |
| ATOM | 3392 | CG | LYS | B | 633 | 43.316 | 33.272 | 22.310 | 1.00 | 33.19 |
| ATOM | 3393 | CD | LYS | B | 633 | 44.524 | 32.424 | 22.662 | 1.00 | 33.59 |
| ATOM | 3394 | CE | LYS | B | 633 | 45.416 | 33.167 | 23.643 | 1.00 | 33.78 |
| ATOM | 3395 | NZ | LYS | B | 633 | 45.749 | 34.527 | 23.130 | 1.00 | 34.10 |
| ATOM | 3396 | C | LYS | B | 633 | 40.709 | 32.888 | 19.499 | 1.00 | 24.23 |
| ATOM | 3397 | O | LYS | B | 633 | 41.243 | 31.935 | 18.928 | 1.00 | 24.88 |
| ATOM | 3398 | N | ILE | B | 634 | 39.565 | 33.411 | 19.079 | 1.00 | 22.51 |
| ATOM | 3399 | CA | ILE | B | 634 | 38.911 | 32.861 | 17.907 | 1.00 | 20.76 |
| ATOM | 3400 | CB | ILE | B | 634 | 37.376 | 32.982 | 18.036 | 1.00 | 22.93 |
| ATOM | 3401 | CG2 | ILE | B | 634 | 36.694 | 32.531 | 16.750 | 1.00 | 23.15 |
| ATOM | 3402 | CG1 | ILE | B | 634 | 36.907 | 32.122 | 19.218 | 1.00 | 22.60 |
| ATOM | 3403 | CD1 | ILE | B | 634 | 35.410 | 32.116 | 19.444 | 1.00 | 24.49 |
| ATOM | 3404 | C | ILE | B | 634 | 39.424 | 33.612 | 16.680 | 1.00 | 21.02 |
| ATOM | 3405 | O | ILE | B | 634 | 39.394 | 33.109 | 15.559 | 1.00 | 21.18 |
| ATOM | 3406 | N | GLN | B | 635 | 39.920 | 34.821 | 16.911 | 1.00 | 20.73 |
| ATOM | 3407 | CA | GLN | B | 635 | 40.453 | 35.656 | 15.848 | 1.00 | 21.87 |
| ATOM | 3408 | CB | GLN | B | 635 | 41.023 | 36.933 | 16.464 | 1.00 | 28.05 |
| ATOM | 3409 | CG | GLN | B | 635 | 41.703 | 37.868 | 15.503 | 1.00 | 29.39 |
| ATOM | 3410 | CD | GLN | B | 635 | 42.362 | 39.034 | 16.217 | 1.00 | 27.87 |
| ATOM | 3411 | OE1 | GLN | B | 635 | 43.020 | 39.856 | 15.596 | 1.00 | 29.76 |
| ATOM | 3412 | NE2 | GLN | B | 635 | 42.178 | 39.110 | 17.532 | 1.00 | 27.16 |
| ATOM | 3413 | C | GLN | B | 635 | 41.543 | 34.904 | 15.089 | 1.00 | 22.14 |
| ATOM | 3414 | O | GLN | B | 635 | 41.600 | 34.934 | 13.858 | 1.00 | 21.83 |
| ATOM | 3415 | N | ASN | B | 636 | 42.387 | 34.224 | 15.854 | 1.00 | 23.53 |
| ATOM | 3416 | CA | ASN | B | 636 | 43.504 | 33.447 | 15.335 | 1.00 | 24.32 |
| ATOM | 3417 | CB | ASN | B | 636 | 44.216 | 32.735 | 16.486 | 1.00 | 22.54 |
| ATOM | 3418 | CG | ASN | B | 636 | 44.607 | 33.675 | 17.606 | 1.00 | 22.42 |
| ATOM | 3419 | OD1 | ASN | B | 636 | 45.788 | 33.821 | 17.921 | 1.00 | 23.23 |
| ATOM | 3420 | ND2 | ASN | B | 636 | 43.620 | 34.307 | 18.221 | 1.00 | 21.19 |
| ATOM | 3421 | C | ASN | B | 636 | 43.089 | 32.390 | 14.325 | 1.00 | 24.28 |
| ATOM | 3422 | O | ASN | B | 636 | 43.868 | 32.022 | 13.443 | 1.00 | 24.97 |
| ATOM | 3423 | N | LYS | B | 637 | 41.864 | 31.899 | 14.463 | 1.00 | 22.71 |
| ATOM | 3424 | CA | LYS | B | 637 | 41.355 | 30.842 | 13.597 | 1.00 | 22.76 |
| ATOM | 3425 | CB | LYS | B | 637 | 40.417 | 29.948 | 14.409 | 1.00 | 25.00 |
| ATOM | 3426 | CG | LYS | B | 637 | 41.028 | 29.410 | 15.688 | 1.00 | 25.88 |
| ATOM | 3427 | CD | LYS | B | 637 | 39.983 | 28.674 | 16.515 | 1.00 | 24.87 |
| ATOM | 3428 | CE | LYS | B | 637 | 40.570 | 28.186 | 17.820 | 1.00 | 26.38 |
| ATOM | 3429 | NZ | LYS | B | 637 | 41.716 | 27.261 | 17.598 | 1.00 | 26.67 |
| ATOM | 3430 | C | LYS | B | 637 | 40.630 | 31.305 | 12.341 | 1.00 | 22.83 |
| ATOM | 3431 | O | LYS | B | 637 | 40.289 | 30.488 | 11.483 | 1.00 | 22.28 |
| ATOM | 3432 | N | LEU | B | 638 | 40.407 | 32.610 | 12.218 | 1.00 | 21.88 |
| ATOM | 3433 | CA | LEU | B | 638 | 39.669 | 33.131 | 11.081 | 1.00 | 22.21 |
| ATOM | 3434 | CB | LEU | B | 638 | 38.421 | 33.855 | 11.601 | 1.00 | 18.60 |
| ATOM | 3435 | CG | LEU | B | 638 | 37.598 | 33.093 | 12.650 | 1.00 | 20.22 |
| ATOM | 3436 | CD1 | LEU | B | 638 | 36.507 | 34.001 | 13.212 | 1.00 | 18.99 |
| ATOM | 3437 | CD2 | LEU | B | 638 | 36.985 | 31.847 | 12.035 | 1.00 | 19.54 |
| ATOM | 3438 | C | LEU | B | 638 | 40.447 | 34.061 | 10.160 | 1.00 | 21.99 |
| ATOM | 3439 | O | LEU | B | 638 | 41.531 | 34.549 | 10.502 | 1.00 | 21.62 |
| ATOM | 3440 | N | THR | B | 639 | 39.878 | 34.299 | 8.982 | 1.00 | 19.43 |
| ATOM | 3441 | CA | THR | B | 639 | 40.474 | 35.188 | 7.998 | 1.00 | 20.58 |
| ATOM | 3442 | CB | THR | B | 639 | 39.900 | 34.937 | 6.593 | 1.00 | 24.74 |
| ATOM | 3443 | OG1 | THR | B | 639 | 38.501 | 35.257 | 6.581 | 1.00 | 23.76 |
| ATOM | 3444 | CG2 | THR | B | 639 | 40.075 | 33.472 | 6.195 | 1.00 | 24.89 |
| ATOM | 3445 | C | THR | B | 639 | 40.110 | 36.609 | 8.413 | 1.00 | 20.43 |
| ATOM | 3446 | O | THR | B | 639 | 39.185 | 36.802 | 9.203 | 1.00 | 20.14 |
| ATOM | 3447 | N | ASP | B | 640 | 40.828 | 37.594 | 7.884 | 1.00 | 22.11 |
| ATOM | 3448 | CA | ASP | B | 640 | 40.550 | 38.989 | 8.195 | 1.00 | 21.37 |
| ATOM | 3449 | CB | ASP | B | 640 | 41.576 | 39.908 | 7.528 | 1.00 | 24.51 |
| ATOM | 3450 | CG | ASP | B | 640 | 42.964 | 39.763 | 8.119 | 1.00 | 24.64 |
| ATOM | 3451 | OD1 | ASP | B | 640 | 43.900 | 40.381 | 7.575 | 1.00 | 23.86 |
| ATOM | 3452 | OD2 | ASP | B | 640 | 43.118 | 39.040 | 9.124 | 1.00 | 24.36 |
| ATOM | 3453 | C | ASP | B | 640 | 39.150 | 39.360 | 7.718 | 1.00 | 22.46 |
| ATOM | 3454 | O | ASP | B | 640 | 38.440 | 40.094 | 8.398 | 1.00 | 21.08 |
| ATOM | 3455 | N | LEU | B | 641 | 38.761 | 38.850 | 6.549 | 1.00 | 22.62 |
| ATOM | 3456 | CA | LEU | B | 641 | 37.436 | 39.127 | 5.988 | 1.00 | 22.81 |
| ATOM | 3457 | CB | LEU | B | 641 | 37.312 | 38.504 | 4.594 | 1.00 | 24.97 |
| ATOM | 3458 | CG | LEU | B | 641 | 37.449 | 39.409 | 3.365 | 1.00 | 27.28 |
| ATOM | 3459 | CD1 | LEU | B | 641 | 38.476 | 40.500 | 3.601 | 1.00 | 25.76 |
| ATOM | 3460 | CD2 | LEU | B | 641 | 37.793 | 38.557 | 2.152 | 1.00 | 25.23 |
| ATOM | 3461 | C | LEU | B | 641 | 36.338 | 38.574 | 6.888 | 1.00 | 22.25 |
| ATOM | 3462 | O | LEU | B | 641 | 35.295 | 39.204 | 7.086 | 1.00 | 21.31 |
| ATOM | 3463 | N | GLU | B | 642 | 36.561 | 37.379 | 7.417 | 1.00 | 20.49 |
| ATOM | 3464 | CA | GLU | B | 642 | 35.588 | 36.765 | 8.301 | 1.00 | 20.51 |
| ATOM | 3465 | CB | GLU | B | 642 | 36.005 | 35.327 | 8.615 | 1.00 | 24.37 |
| ATOM | 3466 | CG | GLU | B | 642 | 35.704 | 34.391 | 7.460 | 1.00 | 24.67 |
| ATOM | 3467 | CD | GLU | B | 642 | 36.405 | 33.053 | 7.574 | 1.00 | 24.64 |
| ATOM | 3468 | OE1 | GLU | B | 642 | 36.092 | 32.159 | 6.760 | 1.00 | 27.01 |
| ATOM | 3469 | OE2 | GLU | B | 642 | 37.267 | 32.893 | 8.461 | 1.00 | 24.86 |
| ATOM | 3470 | C | GLU | B | 642 | 35.427 | 37.579 | 9.581 | 1.00 | 20.92 |
| ATOM | 3471 | O | GLU | B | 642 | 34.309 | 37.764 | 10.069 | 1.00 | 20.75 |
| ATOM | 3472 | N | ILE | B | 643 | 36.542 | 38.076 | 10.108 | 1.00 | 21.11 |
| ATOM | 3473 | CA | ILE | B | 643 | 36.529 | 38.880 | 11.325 | 1.00 | 19.45 |
| ATOM | 3474 | CB | ILE | B | 643 | 37.973 | 39.195 | 11.771 | 1.00 | 22.34 |
| ATOM | 3475 | CG2 | ILE | B | 643 | 37.970 | 40.210 | 12.912 | 1.00 | 21.69 |
| ATOM | 3476 | CG1 | ILE | B | 643 | 38.652 | 37.895 | 12.217 | 1.00 | 20.78 |
| ATOM | 3477 | CD1 | ILE | B | 643 | 40.165 | 37.993 | 12.357 | 1.00 | 22.35 |
| ATOM | 3478 | C | ILE | B | 643 | 35.762 | 40.177 | 11.055 | 1.00 | 19.94 |
| ATOM | 3479 | O | ILE | B | 643 | 34.921 | 40.598 | 11.858 | 1.00 | 19.49 |
| ATOM | 3480 | N | LEU | B | 644 | 36.044 | 40.790 | 9.911 | 1.00 | 19.97 |
| ATOM | 3481 | CA | LEU | B | 644 | 35.385 | 42.027 | 9.502 | 1.00 | 19.99 |
| ATOM | 3482 | CB | LEU | B | 644 | 35.926 | 42.466 | 8.135 | 1.00 | 21.41 |
| ATOM | 3483 | CG | LEU | B | 644 | 35.277 | 43.650 | 7.415 | 1.00 | 21.06 |
| ATOM | 3484 | CD1 | LEU | B | 644 | 35.449 | 44.915 | 8.234 | 1.00 | 22.38 |
| ATOM | 3485 | CD2 | LEU | B | 644 | 35.921 | 43.809 | 6.042 | 1.00 | 21.65 |
| ATOM | 3486 | C | LEU | B | 644 | 33.877 | 41.817 | 9.409 | 1.00 | 21.45 |
| ATOM | 3487 | O | LEU | B | 644 | 33.093 | 42.583 | 9.970 | 1.00 | 18.41 |
| ATOM | 3488 | N | ALA | B | 645 | 33.485 | 40.770 | 8.691 | 1.00 | 19.49 |
| ATOM | 3489 | CA | ALA | B | 645 | 32.076 | 40.444 | 8.499 | 1.00 | 20.21 |
| ATOM | 3490 | CB | ALA | B | 645 | 31.940 | 39.266 | 7.539 | 1.00 | 18.61 |
| ATOM | 3491 | C | ALA | B | 645 | 31.371 | 40.125 | 9.806 | 1.00 | 20.78 |
| ATOM | 3492 | O | ALA | B | 645 | 30.229 | 40.535 | 10.009 | 1.00 | 19.80 |
| ATOM | 3493 | N | LEU | B | 646 | 32.047 | 39.395 | 10.688 | 1.00 | 17.92 |
| ATOM | 3494 | CA | LEU | B | 646 | 31.455 | 39.028 | 11.968 | 1.00 | 18.51 |
| ATOM | 3495 | CB | LEU | B | 646 | 32.381 | 38.084 | 12.735 | 1.00 | 21.14 |
| ATOM | 3496 | CG | LEU | B | 646 | 32.393 | 36.631 | 12.250 | 1.00 | 19.75 |
| ATOM | 3497 | CD1 | LEU | B | 646 | 33.560 | 35.903 | 12.894 | 1.00 | 20.35 |
| ATOM | 3498 | CD2 | LEU | B | 646 | 31.077 | 35.951 | 12.587 | 1.00 | 20.12 |
| ATOM | 3499 | C | LEU | B | 646 | 31.138 | 40.241 | 12.837 | 1.00 | 17.72 |
| ATOM | 3500 | O | LEU | B | 646 | 30.075 | 40.304 | 13.460 | 1.00 | 18.91 |
| ATOM | 3501 | N | LEU | B | 647 | 32.054 | 41.202 | 12.893 | 1.00 | 18.38 |
| ATOM | 3502 | CA | LEU | B | 647 | 31.815 | 42.387 | 13.710 | 1.00 | 18.99 |
| ATOM | 3503 | CB | LEU | B | 647 | 33.074 | 43.255 | 13.800 | 1.00 | 16.80 |
| ATOM | 3504 | CG | LEU | B | 647 | 32.940 | 44.461 | 14.734 | 1.00 | 18.47 |
| ATOM | 3505 | CD1 | LEU | B | 647 | 32.637 | 43.978 | 16.146 | 1.00 | 18.48 |
| ATOM | 3506 | CD2 | LEU | B | 647 | 34.221 | 45.278 | 14.707 | 1.00 | 18.50 |
| ATOM | 3507 | C | LEU | B | 647 | 30.666 | 43.201 | 13.128 | 1.00 | 18.90 |
| ATOM | 3508 | O | LEU | B | 647 | 29.776 | 43.646 | 13.861 | 1.00 | 18.98 |
| ATOM | 3509 | N | ILE | B | 648 | 30.689 | 43.399 | 11.813 | 1.00 | 18.80 |
| ATOM | 3510 | CA | ILE | B | 648 | 29.633 | 44.146 | 11.134 | 1.00 | 17.53 |
| ATOM | 3511 | CB | ILE | B | 648 | 29.948 | 44.298 | 9.620 | 1.00 | 17.43 |
| ATOM | 3512 | CG2 | ILE | B | 648 | 28.747 | 44.886 | 8.884 | 1.00 | 16.56 |
| ATOM | 3513 | CG1 | ILE | B | 648 | 31.189 | 45.181 | 9.439 | 1.00 | 16.47 |
| ATOM | 3514 | CD1 | ILE | B | 648 | 31.622 | 45.377 | 7.987 | 1.00 | 16.64 |
| ATOM | 3515 | C | ILE | B | 648 | 28.303 | 43.405 | 11.322 | 1.00 | 19.67 |
| ATOM | 3516 | O | ILE | B | 648 | 27.268 | 44.020 | 11.608 | 1.00 | 19.92 |
| ATOM | 3517 | N | ALA | B | 649 | 28.327 | 42.084 | 11.170 | 1.00 | 17.14 |
| ATOM | 3518 | CA | ALA | B | 649 | 27.105 | 41.297 | 11.335 | 1.00 | 19.06 |
| ATOM | 3519 | CB | ALA | B | 649 | 27.353 | 39.830 | 10.964 | 1.00 | 15.65 |
| ATOM | 3520 | C | ALA | B | 649 | 26.562 | 41.381 | 12.753 | 1.00 | 17.80 |
| ATOM | 3521 | O | ALA | B | 649 | 25.360 | 41.559 | 12.957 | 1.00 | 16.89 |
| ATOM | 3522 | N | ALA | B | 650 | 27.442 | 41.245 | 13.738 | 1.00 | 17.06 |
| ATOM | 3523 | CA | ALA | B | 650 | 27.016 | 41.316 | 15.128 | 1.00 | 16.09 |
| ATOM | 3524 | CB | ALA | B | 650 | 28.219 | 41.166 | 16.056 | 1.00 | 20.12 |
| ATOM | 3525 | C | ALA | B | 650 | 26.299 | 42.641 | 15.406 | 1.00 | 17.22 |
| ATOM | 3526 | O | ALA | B | 650 | 25.239 | 42.660 | 16.031 | 1.00 | 18.13 |
| ATOM | 3527 | N | LEU | B | 651 | 26.875 | 43.739 | 14.935 | 1.00 | 18.31 |
| ATOM | 3528 | CA | LEU | B | 651 | 26.295 | 45.065 | 15.145 | 1.00 | 18.19 |
| ATOM | 3529 | CB | LEU | B | 651 | 27.302 | 46.143 | 14.744 | 1.00 | 17.38 |
| ATOM | 3530 | CG | LEU | B | 651 | 28.532 | 46.318 | 15.633 | 1.00 | 17.82 |
| ATOM | 3531 | CD1 | LEU | B | 651 | 29.580 | 47.146 | 14.917 | 1.00 | 17.72 |
| ATOM | 3532 | CD2 | LEU | B | 651 | 28.108 | 46.993 | 16.939 | 1.00 | 16.75 |
| ATOM | 3533 | C | LEU | B | 651 | 25.010 | 45.305 | 14.363 | 1.00 | 19.12 |
| ATOM | 3534 | O | LEU | B | 651 | 24.145 | 46.073 | 14.792 | 1.00 | 19.03 |
| ATOM | 3535 | N | SER | B | 652 | 24.879 | 44.644 | 13.218 | 1.00 | 16.50 |
| ATOM | 3536 | CA | SER | B | 652 | 23.717 | 44.861 | 12.361 | 1.00 | 15.50 |
| ATOM | 3537 | CB | SER | B | 652 | 24.165 | 45.025 | 10.902 | 1.00 | 19.61 |
| ATOM | 3538 | OG | SER | B | 652 | 25.258 | 45.908 | 10.760 | 1.00 | 18.90 |
| ATOM | 3539 | C | SER | B | 652 | 22.667 | 43.764 | 12.374 | 1.00 | 17.20 |
| ATOM | 3540 | O | SER | B | 652 | 21.616 | 43.932 | 11.770 | 1.00 | 16.98 |
| ATOM | 3541 | N | HIS | B | 653 | 22.922 | 42.664 | 13.073 | 1.00 | 19.06 |
| ATOM | 3542 | CA | HIS | B | 653 | 22.001 | 41.533 | 12.998 | 1.00 | 19.69 |
| ATOM | 3543 | CB | HIS | B | 653 | 22.594 | 40.312 | 13.697 | 1.00 | 17.94 |
| ATOM | 3544 | CG | HIS | B | 653 | 22.332 | 40.270 | 15.165 | 1.00 | 17.54 |
| ATOM | 3545 | CD2 | HIS | B | 653 | 21.396 | 39.612 | 15.888 | 1.00 | 16.38 |
| ATOM | 3546 | ND1 | HIS | B | 653 | 23.064 | 41.010 | 16.066 | 1.00 | 17.02 |
| ATOM | 3547 | CE1 | HIS | B | 653 | 22.592 | 40.809 | 17.282 | 1.00 | 14.84 |
| ATOM | 3548 | NE2 | HIS | B | 653 | 21.582 | 39.965 | 17.202 | 1.00 | 19.75 |
| ATOM | 3549 | C | HIS | B | 653 | 20.548 | 41.689 | 13.430 | 1.00 | 20.16 |
| ATOM | 3550 | O | HIS | B | 653 | 19.734 | 40.833 | 13.106 | 1.00 | 18.45 |
| ATOM | 3551 | N | ASP | B | 654 | 20.206 | 42.762 | 14.143 | 1.00 | 15.51 |
| ATOM | 3552 | CA | ASP | B | 654 | 18.808 | 42.945 | 14.562 | 1.00 | 15.82 |
| ATOM | 3553 | CB | ASP | B | 654 | 18.680 | 42.848 | 16.082 | 1.00 | 17.70 |
| ATOM | 3554 | CG | ASP | B | 654 | 18.401 | 41.444 | 16.565 | 1.00 | 16.46 |
| ATOM | 3555 | OD1 | ASP | B | 654 | 17.724 | 40.681 | 15.845 | 1.00 | 17.13 |
| ATOM | 3556 | OD2 | ASP | B | 654 | 18.826 | 41.109 | 17.689 | 1.00 | 16.53 |
| ATOM | 3557 | C | ASP | B | 654 | 18.246 | 44.300 | 14.120 | 1.00 | 15.98 |
| ATOM | 3558 | O | ASP | B | 654 | 17.249 | 44.775 | 14.679 | 1.00 | 16.02 |
| ATOM | 3559 | N | LEU | B | 655 | 18.875 | 44.903 | 13.113 | 1.00 | 14.12 |
| ATOM | 3560 | CA | LEU | B | 655 | 18.474 | 46.212 | 12.585 | 1.00 | 16.45 |
| ATOM | 3561 | CB | LEU | B | 655 | 19.191 | 46.465 | 11.253 | 1.00 | 22.99 |
| ATOM | 3562 | CG | LEU | B | 655 | 20.250 | 47.557 | 11.043 | 1.00 | 25.93 |
| ATOM | 3563 | CD1 | LEU | B | 655 | 20.660 | 48.211 | 12.341 | 1.00 | 24.18 |
| ATOM | 3564 | CD2 | LEU | B | 655 | 21.435 | 46.946 | 10.315 | 1.00 | 23.72 |
| ATOM | 3565 | C | LEU | B | 655 | 16.968 | 46.355 | 12.374 | 1.00 | 15.99 |
| ATOM | 3566 | O | LEU | B | 655 | 16.332 | 45.488 | 11.777 | 1.00 | 16.81 |
| ATOM | 3567 | N | ASP | B | 656 | 16.403 | 47.452 | 12.877 | 1.00 | 18.46 |
| ATOM | 3568 | CA | ASP | B | 656 | 14.975 | 47.741 | 12.723 | 1.00 | 18.67 |
| ATOM | 3569 | CB | ASP | B | 656 | 14.676 | 48.025 | 11.243 | 1.00 | 19.71 |
| ATOM | 3570 | CG | ASP | B | 656 | 13.318 | 48.677 | 11.028 | 1.00 | 21.79 |
| ATOM | 3571 | OD1 | ASP | B | 656 | 12.968 | 49.591 | 11.803 | 1.00 | 19.47 |
| ATOM | 3572 | OD2 | ASP | B | 656 | 12.612 | 48.282 | 10.075 | 1.00 | 21.77 |
| ATOM | 3573 | C | ASP | B | 656 | 14.046 | 46.647 | 13.244 | 1.00 | 19.00 |
| ATOM | 3574 | O | ASP | B | 656 | 12.965 | 46.434 | 12.697 | 1.00 | 18.21 |
| ATOM | 3575 | N | HIS | B | 657 | 14.460 | 45.952 | 14.298 | 1.00 | 17.23 |
| ATOM | 3576 | CA | HIS | B | 657 | 13.631 | 44.899 | 14.864 | 1.00 | 16.33 |
| ATOM | 3577 | CB | HIS | B | 657 | 14.377 | 44.174 | 15.983 | 1.00 | 19.47 |
| ATOM | 3578 | CG | HIS | B | 657 | 13.705 | 42.915 | 16.419 | 1.00 | 18.64 |
| ATOM | 3579 | CD2 | HIS | B | 657 | 12.475 | 42.701 | 16.941 | 1.00 | 17.65 |
| ATOM | 3580 | ND1 | HIS | B | 657 | 14.300 | 41.673 | 16.318 | 1.00 | 19.33 |
| ATOM | 3581 | CE1 | HIS | B | 657 | 13.467 | 40.752 | 16.763 | 1.00 | 16.32 |
| ATOM | 3582 | NE2 | HIS | B | 657 | 12.352 | 41.349 | 17.147 | 1.00 | 20.76 |
| ATOM | 3583 | C | HIS | B | 657 | 12.352 | 45.546 | 15.409 | 1.00 | 17.90 |
| ATOM | 3584 | O | HIS | B | 657 | 12.419 | 46.529 | 16.151 | 1.00 | 17.75 |
| ATOM | 3585 | N | PRO | B | 658 | 11.176 | 45.011 | 15.034 | 1.00 | 19.30 |
| ATOM | 3586 | CD | PRO | B | 658 | 11.026 | 43.936 | 14.032 | 1.00 | 15.12 |
| ATOM | 3587 | CA | PRO | B | 658 | 9.863 | 45.519 | 15.463 | 1.00 | 20.23 |
| ATOM | 3588 | CB | PRO | B | 658 | 8.922 | 44.970 | 14.398 | 1.00 | 15.90 |
| ATOM | 3589 | CG | PRO | B | 658 | 9.539 | 43.612 | 14.093 | 1.00 | 14.22 |
| ATOM | 3590 | C | PRO | B | 658 | 9.422 | 45.146 | 16.877 | 1.00 | 19.73 |
| ATOM | 3591 | O | PRO | B | 658 | 8.411 | 45.650 | 17.371 | 1.00 | 20.21 |
| ATOM | 3592 | N | GLY | B | 659 | 10.160 | 44.258 | 17.530 | 1.00 | 20.02 |
| ATOM | 3593 | CA | GLY | B | 659 | 9.800 | 43.886 | 18.884 | 1.00 | 20.59 |
| ATOM | 3594 | C | GLY | B | 659 | 8.794 | 42.760 | 18.918 | 1.00 | 20.21 |
| ATOM | 3595 | O | GLY | B | 659 | 8.194 | 42.491 | 19.954 | 1.00 | 21.72 |
| ATOM | 3596 | N | VAL | B | 660 | 8.589 | 42.117 | 17.772 | 1.00 | 20.05 |
| ATOM | 3597 | CA | VAL | B | 660 | 7.685 | 40.980 | 17.675 | 1.00 | 19.03 |
| ATOM | 3598 | CB | VAL | B | 660 | 6.353 | 41.336 | 16.963 | 1.00 | 22.30 |
| ATOM | 3599 | CG1 | VAL | B | 660 | 5.551 | 42.293 | 17.825 | 1.00 | 23.15 |
| ATOM | 3600 | CG2 | VAL | B | 660 | 6.623 | 41.946 | 15.599 | 1.00 | 22.17 |
| ATOM | 3601 | C | VAL | B | 660 | 8.432 | 39.915 | 16.882 | 1.00 | 20.57 |
| ATOM | 3602 | O | VAL | B | 660 | 9.340 | 40.233 | 16.115 | 1.00 | 20.96 |
| ATOM | 3603 | N | SER | B | 661 | 8.043 | 38.660 | 17.068 | 1.00 | 18.27 |
| ATOM | 3604 | CA | SER | B | 661 | 8.707 | 37.525 | 16.413 | 1.00 | 19.30 |
| ATOM | 3605 | CB | SER | B | 661 | 8.450 | 36.256 | 17.217 | 1.00 | 23.22 |
| ATOM | 3606 | OG | SER | B | 661 | 7.108 | 35.823 | 17.058 | 1.00 | 22.03 |
| ATOM | 3607 | C | SER | B | 661 | 8.337 | 37.237 | 14.961 | 1.00 | 19.20 |
| ATOM | 3608 | O | SER | B | 661 | 7.410 | 37.814 | 14.402 | 1.00 | 16.38 |
| ATOM | 3609 | N | ASN | B | 662 | 9.080 | 36.311 | 14.360 | 1.00 | 18.48 |
| ATOM | 3610 | CA | ASN | B | 662 | 8.810 | 35.905 | 12.989 | 1.00 | 17.98 |
| ATOM | 3611 | CB | ASN | B | 662 | 9.832 | 34.868 | 12.521 | 1.00 | 19.15 |
| ATOM | 3612 | CG | ASN | B | 662 | 11.117 | 35.491 | 12.060 | 1.00 | 20.55 |
| ATOM | 3613 | OD1 | ASN | B | 662 | 11.113 | 36.584 | 11.504 | 1.00 | 18.07 |
| ATOM | 3614 | ND2 | ASN | B | 662 | 12.229 | 34.788 | 12.263 | 1.00 | 19.60 |
| ATOM | 3615 | C | ASN | B | 662 | 7.428 | 35.275 | 12.943 | 1.00 | 18.27 |
| ATOM | 3616 | O | ASN | B | 662 | 6.651 | 35.512 | 12.020 | 1.00 | 19.29 |
| ATOM | 3617 | N | GLN | B | 663 | 7.129 | 34.463 | 13.952 | 1.00 | 19.10 |
| ATOM | 3618 | CA | GLN | B | 663 | 5.845 | 33.793 | 14.020 | 1.00 | 19.98 |
| ATOM | 3619 | CB | GLN | B | 663 | 5.763 | 32.909 | 15.267 | 1.00 | 47.82 |
| ATOM | 3620 | CG | GLN | B | 663 | 6.544 | 31.609 | 15.160 | 1.00 | 48.95 |
| ATOM | 3621 | CD | GLN | B | 663 | 8.056 | 31.793 | 15.228 | 1.00 | 49.43 |
| ATOM | 3622 | OE1 | GLN | B | 663 | 8.814 | 30.853 | 14.978 | 1.00 | 49.36 |
| ATOM | 3623 | NE2 | GLN | B | 663 | 8.499 | 32.997 | 15.576 | 1.00 | 48.96 |
| ATOM | 3624 | C | GLN | B | 663 | 4.708 | 34.798 | 14.029 | 1.00 | 19.45 |
| ATOM | 3625 | O | GLN | B | 663 | 3.701 | 34.609 | 13.352 | 1.00 | 19.22 |
| ATOM | 3626 | N | PHE | B | 664 | 4.883 | 35.869 | 14.795 | 1.00 | 17.76 |
| ATOM | 3627 | CA | PHE | B | 664 | 3.866 | 36.909 | 14.888 | 1.00 | 18.71 |
| ATOM | 3628 | CB | PHE | B | 664 | 4.266 | 37.946 | 15.938 | 1.00 | 20.58 |
| ATOM | 3629 | CG | PHE | B | 664 | 3.280 | 39.077 | 16.084 | 1.00 | 20.67 |
| ATOM | 3630 | CD1 | PHE | B | 664 | 3.340 | 40.185 | 15.243 | 1.00 | 20.97 |
| ATOM | 3631 | CD2 | PHE | B | 664 | 2.291 | 39.031 | 17.064 | 1.00 | 20.35 |
| ATOM | 3632 | CE1 | PHE | B | 664 | 2.428 | 41.232 | 15.377 | 1.00 | 20.58 |
| ATOM | 3633 | CE2 | PHE | B | 664 | 1.377 | 40.074 | 17.203 | 1.00 | 21.94 |
| ATOM | 3634 | CZ | PHE | B | 664 | 1.448 | 41.174 | 16.357 | 1.00 | 21.03 |
| ATOM | 3635 | C | PHE | B | 664 | 3.691 | 37.571 | 13.525 | 1.00 | 17.64 |
| ATOM | 3636 | O | PHE | B | 664 | 2.574 | 37.781 | 13.071 | 1.00 | 17.39 |
| ATOM | 3637 | N | LEU | B | 665 | 4.801 | 37.883 | 12.865 | 1.00 | 18.84 |
| ATOM | 3638 | CA | LEU | B | 665 | 4.734 | 38.504 | 11.552 | 1.00 | 20.08 |
| ATOM | 3639 | CB | LEU | B | 665 | 6.146 | 38.866 | 11.078 | 1.00 | 18.16 |
| ATOM | 3640 | CG | LEU | B | 665 | 6.834 | 39.975 | 11.886 | 1.00 | 19.07 |
| ATOM | 3641 | CD1 | LEU | B | 665 | 8.288 | 40.092 | 11.490 | 1.00 | 19.47 |
| ATOM | 3642 | CD2 | LEU | B | 665 | 6.119 | 41.289 | 11.654 | 1.00 | 18.80 |
| ATOM | 3643 | C | LEU | B | 665 | 4.039 | 37.582 | 10.540 | 1.00 | 20.00 |
| ATOM | 3644 | O | LEU | B | 665 | 3.350 | 38.044 | 9.627 | 1.00 | 21.88 |
| ATOM | 3645 | N | ILE | B | 666 | 4.222 | 36.276 | 10.700 | 1.00 | 18.71 |
| ATOM | 3646 | CA | ILE | B | 666 | 3.593 | 35.317 | 9.806 | 1.00 | 19.12 |
| ATOM | 3647 | CB | ILE | B | 666 | 4.239 | 33.920 | 9.951 | 1.00 | 22.08 |
| ATOM | 3648 | CG2 | ILE | B | 666 | 3.423 | 32.879 | 9.199 | 1.00 | 21.74 |
| ATOM | 3649 | CG1 | ILE | B | 666 | 5.674 | 33.967 | 9.421 | 1.00 | 21.76 |
| ATOM | 3650 | CD1 | ILE | B | 666 | 6.456 | 32.684 | 9.619 | 1.00 | 21.29 |
| ATOM | 3651 | C | ILE | B | 666 | 2.104 | 35.218 | 10.119 | 1.00 | 18.58 |
| ATOM | 3652 | O | ILE | B | 666 | 1.274 | 35.193 | 9.211 | 1.00 | 20.21 |
| ATOM | 3653 | N | ASN | B | 667 | 1.773 | 35.183 | 11.408 | 1.00 | 21.92 |
| ATOM | 3654 | CA | ASN | B | 667 | 0.378 | 35.072 | 11.837 | 1.00 | 22.79 |
| ATOM | 3655 | CB | ASN | B | 667 | 0.295 | 34.756 | 13.334 | 1.00 | 23.85 |
| ATOM | 3656 | CG | ASN | B | 667 | 0.779 | 33.354 | 13.669 | 1.00 | 23.91 |
| ATOM | 3657 | OD1 | ASN | B | 667 | 0.712 | 32.444 | 12.843 | 1.00 | 24.94 |
| ATOM | 3658 | ND2 | ASN | B | 667 | 1.250 | 33.173 | 14.895 | 1.00 | 23.83 |
| ATOM | 3659 | C | ASN | B | 667 | −0.472 | 36.305 | 11.544 | 1.00 | 23.14 |
| ATOM | 3660 | O | ASN | B | 667 | −1.702 | 36.213 | 11.470 | 1.00 | 22.85 |
| ATOM | 3661 | N | THR | B | 668 | 0.169 | 37.459 | 11.392 | 1.00 | 24.81 |
| ATOM | 3662 | CA | THR | B | 668 | −0.566 | 38.677 | 11.086 | 1.00 | 25.96 |
| ATOM | 3663 | CB | THR | B | 668 | −0.019 | 39.894 | 11.882 | 1.00 | 21.17 |
| ATOM | 3664 | OG1 | THR | B | 668 | 1.404 | 39.984 | 11.714 | 1.00 | 23.05 |
| ATOM | 3665 | CG2 | THR | B | 668 | −0.338 | 39.753 | 13.362 | 1.00 | 22.09 |
| ATOM | 3666 | C | THR | B | 668 | −0.511 | 38.973 | 9.585 | 1.00 | 26.69 |
| ATOM | 3667 | O | THR | B | 668 | −0.961 | 40.022 | 9.139 | 1.00 | 27.70 |
| ATOM | 3668 | N | ASN | B | 669 | 0.026 | 38.030 | 8.815 | 1.00 | 26.73 |
| ATOM | 3669 | CA | ASN | B | 669 | 0.138 | 38.169 | 7.362 | 1.00 | 27.16 |
| ATOM | 3670 | CB | ASN | B | 669 | −1.255 | 38.135 | 6.726 | 1.00 | 36.60 |
| ATOM | 3671 | CG | ASN | B | 669 | −1.940 | 36.796 | 6.906 | 1.00 | 36.71 |
| ATOM | 3672 | OD1 | ASN | B | 669 | −1.482 | 35.775 | 6.387 | 1.00 | 36.69 |
| ATOM | 3673 | ND2 | ASN | B | 669 | −3.039 | 36.789 | 7.650 | 1.00 | 37.50 |
| ATOM | 3674 | C | ASN | B | 669 | 0.867 | 39.453 | 6.978 | 1.00 | 27.24 |
| ATOM | 3675 | O | ASN | B | 669 | 0.440 | 40.195 | 6.088 | 1.00 | 26.93 |
| ATOM | 3676 | N | SER | B | 670 | 1.981 | 39.696 | 7.659 | 1.00 | 23.11 |
| ATOM | 3677 | CA | SER | B | 670 | 2.802 | 40.876 | 7.435 | 1.00 | 22.67 |
| ATOM | 3678 | CB | SER | B | 670 | 3.949 | 40.898 | 8.448 | 1.00 | 34.05 |
| ATOM | 3679 | OG | SER | B | 670 | 4.874 | 41.925 | 8.154 | 1.00 | 35.41 |
| ATOM | 3680 | C | SER | B | 670 | 3.382 | 40.916 | 6.028 | 1.00 | 22.16 |
| ATOM | 3681 | O | SER | B | 670 | 3.580 | 39.880 | 5.388 | 1.00 | 22.16 |
| ATOM | 3682 | N | GLU | B | 671 | 3.662 | 42.125 | 5.548 | 1.00 | 21.76 |
| ATOM | 3683 | CA | GLU | B | 671 | 4.236 | 42.279 | 4.225 | 1.00 | 21.00 |
| ATOM | 3684 | CB | GLU | B | 671 | 4.388 | 43.757 | 3.872 | 1.00 | 26.81 |
| ATOM | 3685 | CG | GLU | B | 671 | 4.799 | 43.990 | 2.433 | 1.00 | 26.53 |
| ATOM | 3686 | CD | GLU | B | 671 | 4.963 | 45.461 | 2.099 | 1.00 | 27.67 |
| ATOM | 3687 | OE1 | GLU | B | 671 | 6.052 | 46.018 | 2.361 | 1.00 | 28.24 |
| ATOM | 3688 | OE2 | GLU | B | 671 | 3.995 | 46.062 | 1.584 | 1.00 | 26.44 |
| ATOM | 3689 | C | GLU | B | 671 | 5.607 | 41.617 | 4.228 | 1.00 | 20.25 |
| ATOM | 3690 | O | GLU | B | 671 | 5.996 | 40.962 | 3.271 | 1.00 | 20.37 |
| ATOM | 3691 | N | LEU | B | 672 | 6.332 | 41.799 | 5.323 | 1.00 | 20.82 |
| ATOM | 3692 | CA | LEU | B | 672 | 7.664 | 41.222 | 5.476 | 1.00 | 21.28 |
| ATOM | 3693 | CB | LEU | B | 672 | 8.189 | 41.544 | 6.872 | 1.00 | 29.67 |
| ATOM | 3694 | CG | LEU | B | 672 | 9.522 | 42.279 | 7.026 | 1.00 | 30.73 |
| ATOM | 3695 | CD1 | LEU | B | 672 | 9.670 | 43.391 | 6.003 | 1.00 | 30.03 |
| ATOM | 3696 | CD2 | LEU | B | 672 | 9.588 | 42.833 | 8.438 | 1.00 | 30.18 |
| ATOM | 3697 | C | LEU | B | 672 | 7.610 | 39.711 | 5.277 | 1.00 | 21.18 |
| ATOM | 3698 | O | LEU | B | 672 | 8.470 | 39.121 | 4.620 | 1.00 | 20.85 |
| ATOM | 3699 | N | ALA | B | 673 | 6.585 | 39.084 | 5.844 | 1.00 | 21.74 |
| ATOM | 3700 | CA | ALA | B | 673 | 6.435 | 37.635 | 5.726 | 1.00 | 21.93 |
| ATOM | 3701 | CB | ALA | B | 673 | 5.285 | 37.150 | 6.607 | 1.00 | 19.72 |
| ATOM | 3702 | C | ALA | B | 673 | 6.202 | 37.211 | 4.282 | 1.00 | 22.57 |
| ATOM | 3703 | O | ALA | B | 673 | 6.723 | 36.189 | 3.839 | 1.00 | 21.41 |
| ATOM | 3704 | N | LEU | B | 674 | 5.410 | 37.987 | 3.548 | 1.00 | 20.77 |
| ATOM | 3705 | CA | LEU | B | 674 | 5.151 | 37.664 | 2.153 | 1.00 | 21.89 |
| ATOM | 3706 | CB | LEU | B | 674 | 4.078 | 38.586 | 1.572 | 1.00 | 30.80 |
| ATOM | 3707 | CG | LEU | B | 674 | 3.816 | 38.413 | 0.069 | 1.00 | 31.55 |
| ATOM | 3708 | CD1 | LEU | B | 674 | 3.312 | 37.005 | −0.217 | 1.00 | 31.01 |
| ATOM | 3709 | CD2 | LEU | B | 674 | 2.799 | 39.449 | −0.391 | 1.00 | 30.99 |
| ATOM | 3710 | C | LEU | B | 674 | 6.431 | 37.811 | 1.336 | 1.00 | 22.16 |
| ATOM | 3711 | O | LEU | B | 674 | 6.775 | 36.929 | 0.552 | 1.00 | 21.85 |
| ATOM | 3712 | N | MET | B | 675 | 7.132 | 38.922 | 1.536 | 1.00 | 23.84 |
| ATOM | 3713 | CA | MET | B | 675 | 8.366 | 39.192 | 0.809 | 1.00 | 25.40 |
| ATOM | 3714 | CB | MET | B | 675 | 8.979 | 40.513 | 1.278 | 1.00 | 42.14 |
| ATOM | 3715 | CG | MET | B | 675 | 8.153 | 41.746 | 0.946 | 1.00 | 42.93 |
| ATOM | 3716 | SD | MET | B | 675 | 8.869 | 43.254 | 1.635 | 1.00 | 43.64 |
| ATOM | 3717 | CE | MET | B | 675 | 10.093 | 43.646 | 0.397 | 1.00 | 43.26 |
| ATOM | 3718 | C | MET | B | 675 | 9.397 | 38.080 | 0.973 | 1.00 | 24.69 |
| ATOM | 3719 | O | MET | B | 675 | 10.072 | 37.706 | 0.010 | 1.00 | 25.28 |
| ATOM | 3720 | N | TYR | B | 676 | 9.514 | 37.546 | 2.186 | 1.00 | 22.91 |
| ATOM | 3721 | CA | TYR | B | 676 | 10.503 | 36.506 | 2.449 | 1.00 | 23.58 |
| ATOM | 3722 | CB | TYR | B | 676 | 11.316 | 36.899 | 3.687 | 1.00 | 22.54 |
| ATOM | 3723 | CG | TYR | B | 676 | 11.987 | 38.248 | 3.527 | 1.00 | 23.06 |
| ATOM | 3724 | CD1 | TYR | B | 676 | 11.763 | 39.276 | 4.437 | 1.00 | 23.12 |
| ATOM | 3725 | CE1 | TYR | B | 676 | 12.338 | 40.539 | 4.259 | 1.00 | 22.13 |
| ATOM | 3726 | CD2 | TYR | B | 676 | 12.814 | 38.508 | 2.432 | 1.00 | 24.18 |
| ATOM | 3727 | CE2 | TYR | B | 676 | 13.400 | 39.762 | 2.247 | 1.00 | 22.56 |
| ATOM | 3728 | CZ | TYR | B | 676 | 13.155 | 40.772 | 3.161 | 1.00 | 23.95 |
| ATOM | 3729 | OH | TYR | B | 676 | 13.719 | 42.015 | 2.974 | 1.00 | 23.51 |
| ATOM | 3730 | C | TYR | B | 676 | 9.973 | 35.074 | 2.578 | 1.00 | 22.94 |
| ATOM | 3731 | O | TYR | B | 676 | 10.640 | 34.204 | 3.144 | 1.00 | 23.18 |
| ATOM | 3732 | N | ASN | B | 677 | 8.781 | 34.840 | 2.034 | 1.00 | 23.86 |
| ATOM | 3733 | CA | ASN | B | 677 | 8.137 | 33.524 | 2.034 | 1.00 | 25.01 |
| ATOM | 3734 | CB | ASN | B | 677 | 8.760 | 32.633 | 0.948 | 1.00 | 56.05 |
| ATOM | 3735 | CG | ASN | B | 677 | 10.243 | 32.381 | 1.169 | 1.00 | 57.14 |
| ATOM | 3736 | OD1 | ASN | B | 677 | 10.642 | 31.796 | 2.177 | 1.00 | 57.41 |
| ATOM | 3737 | ND2 | ASN | B | 677 | 11.069 | 32.819 | 0.222 | 1.00 | 57.47 |
| ATOM | 3738 | C | ASN | B | 677 | 8.139 | 32.783 | 3.373 | 1.00 | 24.18 |
| ATOM | 3739 | O | ASN | B | 677 | 8.385 | 31.575 | 3.430 | 1.00 | 22.27 |
| ATOM | 3740 | N | ASP | B | 678 | 7.850 | 33.519 | 4.442 | 1.00 | 20.57 |
| ATOM | 3741 | CA | ASP | B | 678 | 7.783 | 32.970 | 5.794 | 1.00 | 20.56 |
| ATOM | 3742 | CB | ASP | B | 678 | 6.692 | 31.895 | 5.876 | 1.00 | 24.90 |
| ATOM | 3743 | CG | ASP | B | 678 | 5.310 | 32.442 | 5.609 | 1.00 | 26.44 |
| ATOM | 3744 | OD1 | ASP | B | 678 | 5.139 | 33.676 | 5.661 | 1.00 | 24.56 |
| ATOM | 3745 | OD2 | ASP | B | 678 | 4.386 | 31.632 | 5.359 | 1.00 | 26.91 |
| ATOM | 3746 | C | ASP | B | 678 | 9.074 | 32.390 | 6.358 | 1.00 | 19.72 |
| ATOM | 3747 | O | ASP | B | 678 | 9.067 | 31.843 | 7.464 | 1.00 | 21.82 |
| ATOM | 3748 | N | GLU | B | 679 | 10.175 | 32.513 | 5.625 | 1.00 | 18.31 |
| ATOM | 3749 | CA | GLU | B | 679 | 11.452 | 31.964 | 6.086 | 1.00 | 18.54 |
| ATOM | 3750 | CB | GLU | B | 679 | 12.148 | 31.211 | 4.955 | 1.00 | 42.65 |
| ATOM | 3751 | CG | GLU | B | 679 | 11.459 | 29.942 | 4.518 | 1.00 | 43.73 |
| ATOM | 3752 | CD | GLU | B | 679 | 12.276 | 29.188 | 3.492 | 1.00 | 44.02 |
| ATOM | 3753 | OE1 | GLU | B | 679 | 12.480 | 29.723 | 2.381 | 1.00 | 44.05 |
| ATOM | 3754 | OE2 | GLU | B | 679 | 12.723 | 28.066 | 3.803 | 1.00 | 44.29 |
| ATOM | 3755 | C | GLU | B | 679 | 12.407 | 33.021 | 6.611 | 1.00 | 18.73 |
| ATOM | 3756 | O | GLU | B | 679 | 12.768 | 33.939 | 5.875 | 1.00 | 19.73 |
| ATOM | 3757 | N | SER | B | 680 | 12.832 | 32.866 | 7.867 | 1.00 | 18.88 |
| ATOM | 3758 | CA | SER | B | 680 | 13.758 | 33.798 | 8.506 | 1.00 | 16.49 |
| ATOM | 3759 | CB | SER | B | 680 | 15.197 | 33.508 | 8.065 | 1.00 | 21.45 |
| ATOM | 3760 | OG | SER | B | 680 | 15.556 | 32.162 | 8.358 | 1.00 | 20.83 |
| ATOM | 3761 | C | SER | B | 680 | 13.383 | 35.226 | 8.127 | 1.00 | 16.21 |
| ATOM | 3762 | O | SER | B | 680 | 14.232 | 36.019 | 7.722 | 1.00 | 14.77 |
| ATOM | 3763 | N | VAL | B | 681 | 12.099 | 35.536 | 8.272 | 1.00 | 17.92 |
| ATOM | 3764 | CA | VAL | B | 681 | 11.558 | 36.838 | 7.904 | 1.00 | 16.01 |
| ATOM | 3765 | CB | VAL | B | 681 | 10.084 | 36.928 | 8.328 | 1.00 | 18.23 |
| ATOM | 3766 | CG1 | VAL | B | 681 | 9.501 | 38.295 | 7.956 | 1.00 | 18.53 |
| ATOM | 3767 | CG2 | VAL | B | 681 | 9.295 | 35.822 | 7.634 | 1.00 | 19.77 |
| ATOM | 3768 | C | VAL | B | 681 | 12.328 | 38.038 | 8.440 | 1.00 | 15.81 |
| ATOM | 3769 | O | VAL | B | 681 | 12.799 | 38.872 | 7.667 | 1.00 | 17.42 |
| ATOM | 3770 | N | LEU | B | 681A | 12.449 | 38.120 | 9.761 | 1.00 | 15.91 |
| ATOM | 3771 | CA | LEU | B | 681A | 13.164 | 39.216 | 10.410 | 1.00 | 17.15 |
| ATOM | 3772 | CB | LEU | B | 681A | 13.094 | 39.055 | 11.925 | 1.00 | 20.96 |
| ATOM | 3773 | CG | LEU | B | 681A | 11.729 | 39.310 | 12.559 | 1.00 | 24.02 |
| ATOM | 3774 | CD1 | LEU | B | 681A | 11.671 | 38.670 | 13.947 | 1.00 | 23.49 |
| ATOM | 3775 | CD2 | LEU | B | 681A | 11.500 | 40.805 | 12.619 | 1.00 | 23.37 |
| ATOM | 3776 | C | LEU | B | 681A | 14.626 | 39.293 | 9.997 | 1.00 | 17.72 |
| ATOM | 3777 | O | LEU | B | 681A | 15.151 | 40.367 | 9.733 | 1.00 | 16.37 |
| ATOM | 3778 | N | GLU | B | 682 | 15.281 | 38.141 | 9.936 | 1.00 | 16.35 |
| ATOM | 3779 | CA | GLU | B | 682 | 16.690 | 38.112 | 9.582 | 1.00 | 16.54 |
| ATOM | 3780 | CB | GLU | B | 682 | 17.226 | 36.693 | 9.797 | 1.00 | 17.48 |
| ATOM | 3781 | CG | GLU | B | 682 | 17.172 | 36.219 | 11.264 | 1.00 | 19.45 |
| ATOM | 3782 | CD | GLU | B | 682 | 15.765 | 35.939 | 11.783 | 1.00 | 17.96 |
| ATOM | 3783 | OE1 | GLU | B | 682 | 14.873 | 35.587 | 10.975 | 1.00 | 16.02 |
| ATOM | 3784 | OE2 | GLU | B | 682 | 15.553 | 36.043 | 13.015 | 1.00 | 18.67 |
| ATOM | 3785 | C | GLU | B | 682 | 16.957 | 38.616 | 8.162 | 1.00 | 16.65 |
| ATOM | 3786 | O | GLU | B | 682 | 17.956 | 39.307 | 7.908 | 1.00 | 17.06 |
| ATOM | 3787 | N | HIS | B | 683 | 16.068 | 38.286 | 7.233 | 1.00 | 16.83 |
| ATOM | 3788 | CA | HIS | B | 683 | 16.225 | 38.780 | 5.880 | 1.00 | 17.12 |
| ATOM | 3789 | CB | HIS | B | 683 | 15.193 | 38.144 | 4.953 | 1.00 | 21.77 |
| ATOM | 3790 | CG | HIS | B | 683 | 15.579 | 36.779 | 4.481 | 1.00 | 20.98 |
| ATOM | 3791 | CD2 | HIS | B | 683 | 15.140 | 35.552 | 4.845 | 1.00 | 21.60 |
| ATOM | 3792 | ND1 | HIS | B | 683 | 16.550 | 36.571 | 3.524 | 1.00 | 21.61 |
| ATOM | 3793 | CE1 | HIS | B | 683 | 16.690 | 35.274 | 3.317 | 1.00 | 20.39 |
| ATOM | 3794 | NE2 | HIS | B | 683 | 15.847 | 34.634 | 4.107 | 1.00 | 21.29 |
| ATOM | 3795 | C | HIS | B | 683 | 16.050 | 40.291 | 5.923 | 1.00 | 16.91 |
| ATOM | 3796 | O | HIS | B | 683 | 16.745 | 41.017 | 5.220 | 1.00 | 18.17 |
| ATOM | 3797 | N | HIS | B | 684 | 15.136 | 40.756 | 6.772 | 1.00 | 16.37 |
| ATOM | 3798 | CA | HIS | B | 684 | 14.900 | 42.194 | 6.906 | 1.00 | 18.67 |
| ATOM | 3799 | CB | HIS | B | 684 | 13.674 | 42.472 | 7.789 | 1.00 | 18.26 |
| ATOM | 3800 | CG | HIS | B | 684 | 13.376 | 43.934 | 7.962 | 1.00 | 19.15 |
| ATOM | 3801 | CD2 | HIS | B | 684 | 13.327 | 44.714 | 9.068 | 1.00 | 20.53 |
| ATOM | 3802 | ND1 | HIS | B | 684 | 13.124 | 44.769 | 6.896 | 1.00 | 20.23 |
| ATOM | 3803 | CE1 | HIS | B | 684 | 12.939 | 46.002 | 7.336 | 1.00 | 19.60 |
| ATOM | 3804 | NE2 | HIS | B | 684 | 13.056 | 45.996 | 8.650 | 1.00 | 19.94 |
| ATOM | 3805 | C | HIS | B | 684 | 16.131 | 42.858 | 7.516 | 1.00 | 18.24 |
| ATOM | 3806 | O | HIS | B | 684 | 16.556 | 43.927 | 7.066 | 1.00 | 19.27 |
| ATOM | 3807 | N | HIS | B | 685 | 16.706 | 42.228 | 8.537 | 1.00 | 18.19 |
| ATOM | 3808 | CA | HIS | B | 685 | 17.890 | 42.797 | 9.180 | 1.00 | 17.28 |
| ATOM | 3809 | CB | HIS | B | 685 | 18.349 | 41.957 | 10.376 | 1.00 | 17.43 |
| ATOM | 3810 | CG | HIS | B | 685 | 17.301 | 41.751 | 11.425 | 1.00 | 18.40 |
| ATOM | 3811 | CD2 | HIS | B | 685 | 16.971 | 40.654 | 12.148 | 1.00 | 18.57 |
| ATOM | 3812 | ND1 | HIS | B | 685 | 16.479 | 42.765 | 11.871 | 1.00 | 17.46 |
| ATOM | 3813 | CE1 | HIS | B | 685 | 15.689 | 42.299 | 12.823 | 1.00 | 17.20 |
| ATOM | 3814 | NE2 | HIS | B | 685 | 15.967 | 41.022 | 13.011 | 1.00 | 17.24 |
| ATOM | 3815 | C | HIS | B | 685 | 19.028 | 42.908 | 8.175 | 1.00 | 18.93 |
| ATOM | 3816 | O | HIS | B | 685 | 19.725 | 43.919 | 8.140 | 1.00 | 16.49 |
| ATOM | 3817 | N | PHE | B | 686 | 19.227 | 41.870 | 7.363 | 1.00 | 18.43 |
| ATOM | 3818 | CA | PHE | B | 686 | 20.289 | 41.922 | 6.363 | 1.00 | 19.77 |
| ATOM | 3819 | CB | PHE | B | 686 | 20.430 | 40.587 | 5.625 | 1.00 | 22.01 |
| ATOM | 3820 | CG | PHE | B | 686 | 21.424 | 40.635 | 4.501 | 1.00 | 22.74 |
| ATOM | 3821 | CD1 | PHE | B | 686 | 22.771 | 40.879 | 4.757 | 1.00 | 22.40 |
| ATOM | 3822 | CD2 | PHE | B | 686 | 21.008 | 40.509 | 3.185 | 1.00 | 22.19 |
| ATOM | 3823 | CE1 | PHE | B | 686 | 23.689 | 41.003 | 3.714 | 1.00 | 22.33 |
| ATOM | 3824 | CE2 | PHE | B | 686 | 21.915 | 40.631 | 2.139 | 1.00 | 23.22 |
| ATOM | 3825 | CZ | PHE | B | 686 | 23.259 | 40.882 | 2.406 | 1.00 | 22.45 |
| ATOM | 3826 | C | PHE | B | 686 | 20.017 | 43.025 | 5.345 | 1.00 | 19.33 |
| ATOM | 3827 | O | PHE | B | 686 | 20.924 | 43.747 | 4.938 | 1.00 | 19.34 |
| ATOM | 3828 | N | ASP | B | 687 | 18.762 | 43.149 | 4.927 | 1.00 | 20.18 |
| ATOM | 3829 | CA | ASP | B | 687 | 18.390 | 44.175 | 3.974 | 1.00 | 21.34 |
| ATOM | 3830 | CB | ASP | B | 687 | 16.886 | 44.130 | 3.728 | 1.00 | 29.63 |
| ATOM | 3831 | CG | ASP | B | 687 | 16.421 | 45.207 | 2.777 | 1.00 | 30.70 |
| ATOM | 3832 | OD1 | ASP | B | 687 | 16.880 | 45.210 | 1.616 | 1.00 | 31.13 |
| ATOM | 3833 | OD2 | ASP | B | 687 | 15.602 | 46.053 | 3.194 | 1.00 | 32.12 |
| ATOM | 3834 | C | ASP | B | 687 | 18.789 | 45.539 | 4.526 | 1.00 | 20.19 |
| ATOM | 3835 | O | ASP | B | 687 | 19.402 | 46.344 | 3.826 | 1.00 | 20.58 |
| ATOM | 3836 | N | GLN | B | 688 | 18.447 | 45.779 | 5.790 | 1.00 | 19.31 |
| ATOM | 3837 | CA | GLN | B | 688 | 18.773 | 47.044 | 6.447 | 1.00 | 20.14 |
| ATOM | 3838 | CB | GLN | B | 688 | 18.145 | 47.090 | 7.842 | 1.00 | 25.17 |
| ATOM | 3839 | CG | GLN | B | 688 | 16.621 | 47.113 | 7.823 | 1.00 | 26.26 |
| ATOM | 3840 | CD | GLN | B | 688 | 16.074 | 48.399 | 7.241 | 1.00 | 26.81 |
| ATOM | 3841 | OE1 | GLN | B | 688 | 16.218 | 49.471 | 7.835 | 1.00 | 28.04 |
| ATOM | 3842 | NE2 | GLN | B | 688 | 15.456 | 48.306 | 6.071 | 1.00 | 26.91 |
| ATOM | 3843 | C | GLN | B | 688 | 20.282 | 47.246 | 6.549 | 1.00 | 20.35 |
| ATOM | 3844 | O | GLN | B | 688 | 20.777 | 48.366 | 6.389 | 1.00 | 19.51 |
| ATOM | 3845 | N | CYS | B | 689 | 21.003 | 46.159 | 6.811 | 1.00 | 19.79 |
| ATOM | 3846 | CA | CYS | B | 689 | 22.457 | 46.199 | 6.924 | 1.00 | 20.08 |
| ATOM | 3847 | CB | CYS | B | 689 | 22.984 | 44.803 | 7.271 | 1.00 | 19.48 |
| ATOM | 3848 | SG | CYS | B | 689 | 24.795 | 44.663 | 7.336 | 1.00 | 17.52 |
| ATOM | 3849 | C | CYS | B | 689 | 23.085 | 46.679 | 5.617 | 1.00 | 19.76 |
| ATOM | 3850 | O | CYS | B | 689 | 23.949 | 47.563 | 5.605 | 1.00 | 19.63 |
| ATOM | 3851 | N | LEU | B | 690 | 22.655 | 46.072 | 4.517 | 1.00 | 19.56 |
| ATOM | 3852 | CA | LEU | B | 690 | 23.147 | 46.417 | 3.192 | 1.00 | 20.27 |
| ATOM | 3853 | CB | LEU | B | 690 | 22.516 | 45.489 | 2.154 | 1.00 | 27.59 |
| ATOM | 3854 | CG | LEU | B | 690 | 23.083 | 45.572 | 0.739 | 1.00 | 27.68 |
| ATOM | 3855 | CD1 | LEU | B | 690 | 24.542 | 45.123 | 0.746 | 1.00 | 28.21 |
| ATOM | 3856 | CD2 | LEU | B | 690 | 22.257 | 44.692 | −0.183 | 1.00 | 28.78 |
| ATOM | 3857 | C | LEU | B | 690 | 22.795 | 47.867 | 2.872 | 1.00 | 20.36 |
| ATOM | 3858 | O | LEU | B | 690 | 23.607 | 48.606 | 2.332 | 1.00 | 21.09 |
| ATOM | 3859 | N | MET | B | 691 | 21.571 | 48.264 | 3.206 | 1.00 | 21.14 |
| ATOM | 3860 | CA | MET | B | 691 | 21.120 | 49.630 | 2.961 | 1.00 | 22.00 |
| ATOM | 3861 | CB | MET | B | 691 | 19.730 | 49.839 | 3.568 | 1.00 | 37.31 |
| ATOM | 3862 | CG | MET | B | 691 | 19.151 | 51.226 | 3.340 | 1.00 | 37.86 |
| ATOM | 3863 | SD | MET | B | 691 | 17.539 | 51.412 | 4.127 | 1.00 | 38.97 |
| ATOM | 3864 | CE | MET | B | 691 | 18.030 | 51.835 | 5.809 | 1.00 | 38.00 |
| ATOM | 3865 | C | MET | B | 691 | 22.109 | 50.629 | 3.572 | 1.00 | 22.35 |
| ATOM | 3866 | O | MET | B | 691 | 22.552 | 51.559 | 2.902 | 1.00 | 22.19 |
| ATOM | 3867 | N | ILE | B | 692 | 22.455 | 50.420 | 4.839 | 1.00 | 21.40 |
| ATOM | 3868 | CA | ILE | B | 692 | 23.385 | 51.308 | 5.534 | 1.00 | 21.97 |
| ATOM | 3869 | CB | ILE | B | 692 | 23.522 | 50.930 | 7.029 | 1.00 | 25.90 |
| ATOM | 3870 | CG2 | ILE | B | 692 | 24.515 | 51.866 | 7.710 | 1.00 | 24.75 |
| ATOM | 3871 | CG1 | ILE | B | 692 | 22.159 | 51.011 | 7.727 | 1.00 | 26.02 |
| ATOM | 3872 | CD1 | ILE | B | 692 | 21.569 | 52.395 | 7.770 | 1.00 | 26.52 |
| ATOM | 3873 | C | ILE | B | 692 | 24.775 | 51.286 | 4.899 | 1.00 | 22.83 |
| ATOM | 3874 | O | ILE | B | 692 | 25.391 | 52.332 | 4.704 | 1.00 | 21.06 |
| ATOM | 3875 | N | LEU | B | 693 | 25.261 | 50.092 | 4.566 | 1.00 | 21.77 |
| ATOM | 3876 | CA | LEU | B | 693 | 26.581 | 49.951 | 3.951 | 1.00 | 23.08 |
| ATOM | 3877 | CB | LEU | B | 693 | 26.940 | 48.468 | 3.787 | 1.00 | 21.71 |
| ATOM | 3878 | CG | LEU | B | 693 | 27.301 | 47.701 | 5.060 | 1.00 | 21.98 |
| ATOM | 3879 | CD1 | LEU | B | 693 | 27.277 | 46.218 | 4.784 | 1.00 | 22.22 |
| ATOM | 3880 | CD2 | LEU | B | 693 | 28.677 | 48.144 | 5.553 | 1.00 | 22.27 |
| ATOM | 3881 | C | LEU | B | 693 | 26.664 | 50.635 | 2.595 | 1.00 | 23.02 |
| ATOM | 3882 | O | LEU | B | 693 | 27.752 | 51.009 | 2.153 | 1.00 | 24.07 |
| ATOM | 3883 | N | ASN | B | 694 | 25.519 | 50.794 | 1.934 | 1.00 | 24.41 |
| ATOM | 3884 | CA | ASN | B | 694 | 25.478 | 51.437 | 0.626 | 1.00 | 24.18 |
| ATOM | 3885 | CB | ASN | B | 694 | 24.448 | 50.760 | −0.285 | 1.00 | 28.06 |
| ATOM | 3886 | CG | ASN | B | 694 | 24.886 | 49.387 | −0.756 | 1.00 | 28.56 |
| ATOM | 3887 | OD1 | ASN | B | 694 | 26.040 | 49.184 | −1.122 | 1.00 | 29.03 |
| ATOM | 3888 | ND2 | ASN | B | 694 | 23.953 | 48.439 | −0.769 | 1.00 | 28.87 |
| ATOM | 3889 | C | ASN | B | 694 | 25.149 | 52.922 | 0.715 | 1.00 | 25.26 |
| ATOM | 3890 | O | ASN | B | 694 | 25.185 | 53.625 | −0.295 | 1.00 | 25.49 |
| ATOM | 3891 | N | SER | B | 695 | 24.830 | 53.392 | 1.917 | 1.00 | 24.51 |
| ATOM | 3892 | CA | SER | B | 695 | 24.483 | 54.796 | 2.124 | 1.00 | 26.67 |
| ATOM | 3893 | CB | SER | B | 695 | 23.842 | 54.991 | 3.497 | 1.00 | 38.96 |
| ATOM | 3894 | OG | SER | B | 695 | 22.544 | 54.433 | 3.532 | 1.00 | 40.66 |
| ATOM | 3895 | C | SER | B | 695 | 25.679 | 55.724 | 1.999 | 1.00 | 26.23 |
| ATOM | 3896 | O | SER | B | 695 | 26.750 | 55.447 | 2.534 | 1.00 | 25.50 |
| ATOM | 3897 | N | PRO | B | 696 | 25.503 | 56.854 | 1.298 | 1.00 | 30.83 |
| ATOM | 3898 | CD | PRO | B | 696 | 24.242 | 57.397 | 0.766 | 1.00 | 35.10 |
| ATOM | 3899 | CA | PRO | B | 696 | 26.600 | 57.810 | 1.125 | 1.00 | 31.36 |
| ATOM | 3900 | CB | PRO | B | 696 | 25.936 | 58.959 | 0.362 | 1.00 | 35.40 |
| ATOM | 3901 | CG | PRO | B | 696 | 24.499 | 58.882 | 0.813 | 1.00 | 35.60 |
| ATOM | 3902 | C | PRO | B | 696 | 27.205 | 58.251 | 2.461 | 1.00 | 31.40 |
| ATOM | 3903 | O | PRO | B | 696 | 26.480 | 58.569 | 3.406 | 1.00 | 31.21 |
| ATOM | 3904 | N | GLY | B | 697 | 28.535 | 58.250 | 2.528 | 1.00 | 27.19 |
| ATOM | 3905 | CA | GLY | B | 697 | 29.227 | 58.653 | 3.741 | 1.00 | 26.21 |
| ATOM | 3906 | C | GLY | B | 697 | 29.268 | 57.587 | 4.821 | 1.00 | 25.99 |
| ATOM | 3907 | O | GLY | B | 697 | 29.813 | 57.811 | 5.901 | 1.00 | 25.64 |
| ATOM | 3908 | N | ASN | B | 698 | 28.698 | 56.422 | 4.532 | 1.00 | 25.20 |
| ATOM | 3909 | CA | ASN | B | 698 | 28.661 | 55.328 | 5.498 | 1.00 | 24.25 |
| ATOM | 3910 | CB | ASN | B | 698 | 27.215 | 54.970 | 5.867 | 1.00 | 26.49 |
| ATOM | 3911 | CG | ASN | B | 698 | 26.516 | 56.050 | 6.651 | 1.00 | 26.36 |
| ATOM | 3912 | OD1 | ASN | B | 698 | 26.081 | 57.067 | 6.101 | 1.00 | 28.19 |
| ATOM | 3913 | ND2 | ASN | B | 698 | 26.399 | 55.834 | 7.948 | 1.00 | 26.66 |
| ATOM | 3914 | C | ASN | B | 698 | 29.302 | 54.053 | 4.967 | 1.00 | 25.12 |
| ATOM | 3915 | O | ASN | B | 698 | 29.271 | 53.025 | 5.639 | 1.00 | 23.91 |
| ATOM | 3916 | N | GLN | B | 699 | 29.876 | 54.114 | 3.771 | 1.00 | 24.45 |
| ATOM | 3917 | CA | GLN | B | 699 | 30.456 | 52.923 | 3.146 | 1.00 | 24.83 |
| ATOM | 3918 | CB | GLN | B | 699 | 30.510 | 53.143 | 1.633 | 1.00 | 34.19 |
| ATOM | 3919 | CG | GLN | B | 699 | 29.191 | 53.687 | 1.093 | 1.00 | 34.82 |
| ATOM | 3920 | CD | GLN | B | 699 | 29.205 | 53.911 | −0.399 | 1.00 | 35.20 |
| ATOM | 3921 | OE1 | GLN | B | 699 | 29.346 | 52.971 | −1.178 | 1.00 | 35.77 |
| ATOM | 3922 | NE2 | GLN | B | 699 | 29.056 | 55.163 | −0.808 | 1.00 | 35.33 |
| ATOM | 3923 | C | GLN | B | 699 | 31.819 | 52.493 | 3.683 | 1.00 | 23.99 |
| ATOM | 3924 | O | GLN | B | 699 | 32.860 | 52.768 | 3.083 | 1.00 | 25.36 |
| ATOM | 3925 | N | ILE | B | 700 | 31.791 | 51.794 | 4.814 | 1.00 | 20.88 |
| ATOM | 3926 | CA | ILE | B | 700 | 32.996 | 51.309 | 5.475 | 1.00 | 21.48 |
| ATOM | 3927 | CB | ILE | B | 700 | 32.696 | 50.857 | 6.919 | 1.00 | 24.53 |
| ATOM | 3928 | CG2 | ILE | B | 700 | 32.254 | 52.056 | 7.759 | 1.00 | 24.80 |
| ATOM | 3929 | CG1 | ILE | B | 700 | 31.623 | 49.765 | 6.917 | 1.00 | 24.49 |
| ATOM | 3930 | CD1 | ILE | B | 700 | 31.415 | 49.110 | 8.268 | 1.00 | 24.76 |
| ATOM | 3931 | C | ILE | B | 700 | 33.674 | 50.154 | 4.740 | 1.00 | 20.59 |
| ATOM | 3932 | O | ILE | B | 700 | 34.754 | 49.717 | 5.141 | 1.00 | 22.27 |
| ATOM | 3933 | N | LEU | B | 701 | 33.051 | 49.665 | 3.669 | 1.00 | 23.58 |
| ATOM | 3934 | CA | LEU | B | 701 | 33.627 | 48.571 | 2.892 | 1.00 | 23.94 |
| ATOM | 3935 | CB | LEU | B | 701 | 32.609 | 47.433 | 2.711 | 1.00 | 22.71 |
| ATOM | 3936 | CG | LEU | B | 701 | 32.080 | 46.709 | 3.955 | 1.00 | 22.35 |
| ATOM | 3937 | CD1 | LEU | B | 701 | 31.078 | 45.635 | 3.527 | 1.00 | 22.17 |
| ATOM | 3938 | CD2 | LEU | B | 701 | 33.235 | 46.087 | 4.732 | 1.00 | 22.94 |
| ATOM | 3939 | C | LEU | B | 701 | 34.058 | 49.086 | 1.523 | 1.00 | 25.02 |
| ATOM | 3940 | O | LEU | B | 701 | 34.425 | 48.308 | 0.643 | 1.00 | 25.13 |
| ATOM | 3941 | N | SER | B | 702 | 34.020 | 50.404 | 1.354 | 1.00 | 28.81 |
| ATOM | 3942 | CA | SER | B | 702 | 34.383 | 51.035 | 0.087 | 1.00 | 30.23 |
| ATOM | 3943 | CB | SER | B | 702 | 34.177 | 52.549 | 0.174 | 1.00 | 36.19 |
| ATOM | 3944 | OG | SER | B | 702 | 35.139 | 53.137 | 1.032 | 1.00 | 36.39 |
| ATOM | 3945 | C | SER | B | 702 | 35.825 | 50.761 | −0.323 | 1.00 | 30.61 |
| ATOM | 3946 | O | SER | B | 702 | 36.154 | 50.788 | −1.511 | 1.00 | 30.09 |
| ATOM | 3947 | N | GLY | B | 703 | 36.680 | 50.504 | 0.661 | 1.00 | 30.88 |
| ATOM | 3948 | CA | GLY | B | 703 | 38.083 | 50.258 | 0.379 | 1.00 | 30.78 |
| ATOM | 3949 | C | GLY | B | 703 | 38.446 | 48.843 | −0.029 | 1.00 | 31.56 |
| ATOM | 3950 | O | GLY | B | 703 | 39.570 | 48.595 | −0.461 | 1.00 | 31.02 |
| ATOM | 3951 | N | LEU | B | 704 | 37.506 | 47.914 | 0.109 | 1.00 | 27.62 |
| ATOM | 3952 | CA | LEU | B | 704 | 37.748 | 46.522 | −0.252 | 1.00 | 28.14 |
| ATOM | 3953 | CB | LEU | B | 704 | 36.638 | 45.628 | 0.312 | 1.00 | 24.97 |
| ATOM | 3954 | CG | LEU | B | 704 | 36.734 | 45.205 | 1.775 | 1.00 | 25.06 |
| ATOM | 3955 | CD1 | LEU | B | 704 | 35.542 | 44.349 | 2.161 | 1.00 | 25.71 |
| ATOM | 3956 | CD2 | LEU | B | 704 | 38.002 | 44.413 | 1.965 | 1.00 | 26.03 |
| ATOM | 3957 | C | LEU | B | 704 | 37.834 | 46.306 | −1.760 | 1.00 | 27.32 |
| ATOM | 3958 | O | LEU | B | 704 | 37.211 | 47.026 | −2.537 | 1.00 | 27.75 |
| ATOM | 3959 | N | SER | B | 705 | 38.610 | 45.305 | −2.164 | 1.00 | 25.03 |
| ATOM | 3960 | CA | SER | B | 705 | 38.740 | 44.982 | −3.576 | 1.00 | 25.33 |
| ATOM | 3961 | CB | SER | B | 705 | 39.909 | 44.019 | −3.807 | 1.00 | 22.63 |
| ATOM | 3962 | OG | SER | B | 705 | 39.647 | 42.746 | −3.249 | 1.00 | 22.15 |
| ATOM | 3963 | C | SER | B | 705 | 37.428 | 44.308 | −3.952 | 1.00 | 25.55 |
| ATOM | 3964 | O | SER | B | 705 | 36.645 | 43.950 | −3.073 | 1.00 | 25.89 |
| ATOM | 3965 | N | ILE | B | 706 | 37.190 | 44.134 | −5.248 | 1.00 | 28.89 |
| ATOM | 3966 | CA | ILE | B | 706 | 35.960 | 43.503 | −5.712 | 1.00 | 30.40 |
| ATOM | 3967 | CB | ILE | B | 706 | 35.967 | 43.351 | −7.249 | 1.00 | 38.06 |
| ATOM | 3968 | CG2 | ILE | B | 706 | 34.756 | 42.548 | −7.701 | 1.00 | 38.45 |
| ATOM | 3969 | CG1 | ILE | B | 706 | 35.984 | 44.737 | −7.900 | 1.00 | 38.67 |
| ATOM | 3970 | CD1 | ILE | B | 706 | 36.141 | 44.717 | −9.410 | 1.00 | 38.81 |
| ATOM | 3971 | C | ILE | B | 706 | 35.735 | 42.134 | −5.070 | 1.00 | 29.62 |
| ATOM | 3972 | O | ILE | B | 706 | 34.697 | 41.897 | −4.452 | 1.00 | 29.75 |
| ATOM | 3973 | N | GLU | B | 707 | 36.704 | 41.234 | −5.210 | 1.00 | 26.84 |
| ATOM | 3974 | CA | GLU | B | 707 | 36.579 | 39.900 | −4.631 | 1.00 | 26.37 |
| ATOM | 3975 | CB | GLU | B | 707 | 37.811 | 39.056 | −4.958 | 1.00 | 46.28 |
| ATOM | 3976 | CG | GLU | B | 707 | 37.665 | 38.188 | −6.198 | 1.00 | 47.22 |
| ATOM | 3977 | CD | GLU | B | 707 | 36.441 | 37.287 | −6.135 | 1.00 | 47.50 |
| ATOM | 3978 | OE1 | GLU | B | 707 | 35.368 | 37.699 | −6.626 | 1.00 | 47.38 |
| ATOM | 3979 | OE2 | GLU | B | 707 | 36.548 | 36.171 | −5.581 | 1.00 | 47.48 |
| ATOM | 3980 | C | GLU | B | 707 | 36.372 | 39.927 | −3.121 | 1.00 | 25.62 |
| ATOM | 3981 | O | GLU | B | 707 | 35.505 | 39.227 | −2.591 | 1.00 | 24.84 |
| ATOM | 3982 | N | GLU | B | 708 | 37.175 | 40.723 | −2.419 | 1.00 | 24.00 |
| ATOM | 3983 | CA | GLU | B | 708 | 37.031 | 40.803 | −0.974 | 1.00 | 22.95 |
| ATOM | 3984 | CB | GLU | B | 708 | 38.126 | 41.683 | −0.365 | 1.00 | 34.88 |
| ATOM | 3985 | CG | GLU | B | 708 | 39.451 | 40.952 | −0.225 | 1.00 | 34.89 |
| ATOM | 3986 | CD | GLU | B | 708 | 40.418 | 41.646 | 0.714 | 1.00 | 35.05 |
| ATOM | 3987 | OE1 | GLU | B | 708 | 41.368 | 40.981 | 1.167 | 1.00 | 35.66 |
| ATOM | 3988 | OE2 | GLU | B | 708 | 40.235 | 42.846 | 0.994 | 1.00 | 35.78 |
| ATOM | 3989 | C | GLU | B | 708 | 35.654 | 41.327 | −0.579 | 1.00 | 23.54 |
| ATOM | 3990 | O | GLU | B | 708 | 35.049 | 40.824 | 0.368 | 1.00 | 23.08 |
| ATOM | 3991 | N | TYR | B | 709 | 35.169 | 42.332 | −1.302 | 1.00 | 22.88 |
| ATOM | 3992 | CA | TYR | B | 709 | 33.855 | 42.908 | −1.022 | 1.00 | 23.56 |
| ATOM | 3993 | CB | TYR | B | 709 | 33.583 | 44.072 | −1.975 | 1.00 | 28.79 |
| ATOM | 3994 | CG | TYR | B | 709 | 32.228 | 44.708 | −1.784 | 1.00 | 29.56 |
| ATOM | 3995 | CD1 | TYR | B | 709 | 31.950 | 45.476 | −0.656 | 1.00 | 29.43 |
| ATOM | 3996 | CE1 | TYR | B | 709 | 30.691 | 46.039 | −0.461 | 1.00 | 29.53 |
| ATOM | 3997 | CD2 | TYR | B | 709 | 31.214 | 44.516 | −2.718 | 1.00 | 30.32 |
| ATOM | 3998 | CE2 | TYR | B | 709 | 29.950 | 45.072 | −2.533 | 1.00 | 29.63 |
| ATOM | 3999 | CZ | TYR | B | 709 | 29.696 | 45.830 | −1.404 | 1.00 | 30.45 |
| ATOM | 4000 | OH | TYR | B | 709 | 28.447 | 46.375 | −1.221 | 1.00 | 30.77 |
| ATOM | 4001 | C | TYR | B | 709 | 32.764 | 41.841 | −1.174 | 1.00 | 24.17 |
| ATOM | 4002 | O | TYR | B | 709 | 31.909 | 41.677 | −0.297 | 1.00 | 23.90 |
| ATOM | 4003 | N | LYS | B | 710 | 32.800 | 41.117 | −2.289 | 1.00 | 25.48 |
| ATOM | 4004 | CA | LYS | B | 710 | 31.821 | 40.062 | −2.543 | 1.00 | 27.03 |
| ATOM | 4005 | CB | LYS | B | 710 | 32.064 | 39.418 | −3.910 | 1.00 | 49.22 |
| ATOM | 4006 | CG | LYS | B | 710 | 31.519 | 37.999 | −3.992 | 1.00 | 50.09 |
| ATOM | 4007 | CD | LYS | B | 710 | 31.985 | 37.258 | −5.227 | 1.00 | 50.44 |
| ATOM | 4008 | CE | LYS | B | 710 | 31.769 | 35.762 | −5.057 | 1.00 | 50.21 |
| ATOM | 4009 | NZ | LYS | B | 710 | 30.379 | 35.439 | −4.621 | 1.00 | 50.37 |
| ATOM | 4010 | C | LYS | B | 710 | 31.872 | 38.972 | −1.480 | 1.00 | 25.65 |
| ATOM | 4011 | O | LYS | B | 710 | 30.840 | 38.500 | −0.998 | 1.00 | 26.50 |
| ATOM | 4012 | N | THR | B | 711 | 33.081 | 38.556 | −1.128 | 1.00 | 23.83 |
| ATOM | 4013 | CA | THR | B | 711 | 33.249 | 37.511 | −0.135 | 1.00 | 22.67 |
| ATOM | 4014 | CB | THR | B | 711 | 34.727 | 37.120 | −0.008 | 1.00 | 25.01 |
| ATOM | 4015 | OG1 | THR | B | 711 | 35.193 | 36.642 | −1.275 | 1.00 | 25.50 |
| ATOM | 4016 | CG2 | THR | B | 711 | 34.904 | 36.029 | 1.038 | 1.00 | 24.57 |
| ATOM | 4017 | C | THR | B | 711 | 32.723 | 37.944 | 1.232 | 1.00 | 22.92 |
| ATOM | 4018 | O | THR | B | 711 | 32.095 | 37.157 | 1.940 | 1.00 | 23.60 |
| ATOM | 4019 | N | THR | B | 712 | 32.975 | 39.202 | 1.586 | 1.00 | 20.46 |
| ATOM | 4020 | CA | THR | B | 712 | 32.551 | 39.742 | 2.871 | 1.00 | 20.53 |
| ATOM | 4021 | CB | THR | B | 712 | 33.201 | 41.117 | 3.125 | 1.00 | 22.65 |
| ATOM | 4022 | OG1 | THR | B | 712 | 34.624 | 40.993 | 3.023 | 1.00 | 23.36 |
| ATOM | 4023 | CG2 | THR | B | 712 | 32.849 | 41.628 | 4.515 | 1.00 | 22.01 |
| ATOM | 4024 | C | THR | B | 712 | 31.030 | 39.887 | 2.964 | 1.00 | 20.03 |
| ATOM | 4025 | O | THR | B | 712 | 30.433 | 39.564 | 3.992 | 1.00 | 19.56 |
| ATOM | 4026 | N | LEU | B | 713 | 30.405 | 40.375 | 1.899 | 1.00 | 20.53 |
| ATOM | 4027 | CA | LEU | B | 713 | 28.957 | 40.551 | 1.907 | 1.00 | 21.14 |
| ATOM | 4028 | CB | LEU | B | 713 | 28.484 | 41.186 | 0.599 | 1.00 | 32.94 |
| ATOM | 4029 | CG | LEU | B | 713 | 27.729 | 42.511 | 0.729 | 1.00 | 34.20 |
| ATOM | 4030 | CD1 | LEU | B | 713 | 26.628 | 42.370 | 1.774 | 1.00 | 34.24 |
| ATOM | 4031 | CD2 | LEU | B | 713 | 28.684 | 43.616 | 1.134 | 1.00 | 34.00 |
| ATOM | 4032 | C | LEU | B | 713 | 28.263 | 39.212 | 2.116 | 1.00 | 21.40 |
| ATOM | 4033 | O | LEU | B | 713 | 27.265 | 39.117 | 2.834 | 1.00 | 19.21 |
| ATOM | 4034 | N | LYS | B | 714 | 28.795 | 38.171 | 1.488 | 1.00 | 20.01 |
| ATOM | 4035 | CA | LYS | B | 714 | 28.220 | 36.837 | 1.620 | 1.00 | 20.38 |
| ATOM | 4036 | CB | LYS | B | 714 | 28.920 | 35.876 | 0.657 | 1.00 | 40.77 |
| ATOM | 4037 | CG | LYS | B | 714 | 28.564 | 34.419 | 0.847 | 1.00 | 41.45 |
| ATOM | 4038 | CD | LYS | B | 714 | 29.316 | 33.548 | −0.149 | 1.00 | 42.11 |
| ATOM | 4039 | CE | LYS | B | 714 | 29.138 | 32.073 | 0.165 | 1.00 | 42.05 |
| ATOM | 4040 | NZ | LYS | B | 714 | 27.703 | 31.694 | 0.221 | 1.00 | 42.14 |
| ATOM | 4041 | C | LYS | B | 714 | 28.343 | 36.322 | 3.052 | 1.00 | 19.95 |
| ATOM | 4042 | O | LYS | B | 714 | 27.417 | 35.721 | 3.597 | 1.00 | 20.70 |
| ATOM | 4043 | N | ILE | B | 715 | 29.499 | 36.544 | 3.662 | 1.00 | 19.98 |
| ATOM | 4044 | CA | ILE | B | 715 | 29.705 | 36.101 | 5.029 | 1.00 | 18.62 |
| ATOM | 4045 | CB | ILE | B | 715 | 31.145 | 36.386 | 5.500 | 1.00 | 22.51 |
| ATOM | 4046 | CG2 | ILE | B | 715 | 31.317 | 35.943 | 6.939 | 1.00 | 21.50 |
| ATOM | 4047 | CG1 | ILE | B | 715 | 32.141 | 35.638 | 4.612 | 1.00 | 22.82 |
| ATOM | 4048 | CD1 | ILE | B | 715 | 33.571 | 36.088 | 4.812 | 1.00 | 22.76 |
| ATOM | 4049 | C | ILE | B | 715 | 28.732 | 36.838 | 5.955 | 1.00 | 18.65 |
| ATOM | 4050 | O | ILE | B | 715 | 28.190 | 36.245 | 6.882 | 1.00 | 17.75 |
| ATOM | 4051 | N | ILE | B | 716 | 28.528 | 38.127 | 5.693 | 1.00 | 18.42 |
| ATOM | 4052 | CA | ILE | B | 716 | 27.624 | 38.951 | 6.500 | 1.00 | 17.16 |
| ATOM | 4053 | CB | ILE | B | 716 | 27.676 | 40.424 | 6.040 | 1.00 | 19.95 |
| ATOM | 4054 | CG2 | ILE | B | 716 | 26.549 | 41.225 | 6.683 | 1.00 | 19.27 |
| ATOM | 4055 | CG1 | ILE | B | 716 | 29.037 | 41.024 | 6.404 | 1.00 | 18.80 |
| ATOM | 4056 | CD1 | ILE | B | 716 | 29.258 | 42.434 | 5.887 | 1.00 | 18.71 |
| ATOM | 4057 | C | ILE | B | 716 | 26.192 | 38.426 | 6.411 | 1.00 | 19.67 |
| ATOM | 4058 | O | ILE | B | 716 | 25.544 | 38.183 | 7.436 | 1.00 | 16.84 |
| ATOM | 4059 | N | LYS | B | 717 | 25.706 | 38.224 | 5.192 | 1.00 | 17.44 |
| ATOM | 4060 | CA | LYS | B | 717 | 24.344 | 37.721 | 5.040 | 1.00 | 19.23 |
| ATOM | 4061 | CB | LYS | B | 717 | 23.980 | 37.489 | 3.573 | 1.00 | 19.08 |
| ATOM | 4062 | CG | LYS | B | 717 | 22.575 | 36.915 | 3.425 | 1.00 | 19.36 |
| ATOM | 4063 | CD | LYS | B | 717 | 22.145 | 36.779 | 1.975 | 1.00 | 20.95 |
| ATOM | 4064 | CE | LYS | B | 717 | 20.713 | 36.253 | 1.906 | 1.00 | 20.68 |
| ATOM | 4065 | NZ | LYS | B | 717 | 20.177 | 36.245 | 0.520 | 1.00 | 22.57 |
| ATOM | 4066 | C | LYS | B | 717 | 24.167 | 36.408 | 5.797 | 1.00 | 18.60 |
| ATOM | 4067 | O | LYS | B | 717 | 23.204 | 36.240 | 6.539 | 1.00 | 19.65 |
| ATOM | 4068 | N | GLN | B | 718 | 25.091 | 35.469 | 5.602 | 1.00 | 18.67 |
| ATOM | 4069 | CA | GLN | B | 718 | 24.992 | 34.181 | 6.280 | 1.00 | 18.42 |
| ATOM | 4070 | CB | GLN | B | 718 | 26.110 | 33.256 | 5.794 | 1.00 | 35.34 |
| ATOM | 4071 | CG | GLN | B | 718 | 25.885 | 32.724 | 4.386 | 1.00 | 36.06 |
| ATOM | 4072 | CD | GLN | B | 718 | 27.121 | 32.072 | 3.798 | 1.00 | 36.81 |
| ATOM | 4073 | OE1 | GLN | B | 718 | 27.036 | 31.295 | 2.843 | 1.00 | 37.02 |
| ATOM | 4074 | NE2 | GLN | B | 718 | 28.282 | 32.397 | 4.356 | 1.00 | 36.65 |
| ATOM | 4075 | C | GLN | B | 718 | 25.039 | 34.298 | 7.802 | 1.00 | 16.64 |
| ATOM | 4076 | O | GLN | B | 718 | 24.337 | 33.577 | 8.517 | 1.00 | 17.20 |
| ATOM | 4077 | N | ALA | B | 719 | 25.855 | 35.221 | 8.299 | 1.00 | 17.42 |
| ATOM | 4078 | CA | ALA | B | 719 | 25.989 | 35.400 | 9.737 | 1.00 | 16.91 |
| ATOM | 4079 | CB | ALA | B | 719 | 27.181 | 36.314 | 10.037 | 1.00 | 18.95 |
| ATOM | 4080 | C | ALA | B | 719 | 24.705 | 35.979 | 10.338 | 1.00 | 16.46 |
| ATOM | 4081 | O | ALA | B | 719 | 24.280 | 35.566 | 11.415 | 1.00 | 16.41 |
| ATOM | 4082 | N | ILE | B | 720 | 24.087 | 36.923 | 9.632 | 1.00 | 16.16 |
| ATOM | 4083 | CA | ILE | B | 720 | 22.848 | 37.517 | 10.128 | 1.00 | 18.05 |
| ATOM | 4084 | CB | ILE | B | 720 | 22.463 | 38.775 | 9.317 | 1.00 | 16.77 |
| ATOM | 4085 | CG2 | ILE | B | 720 | 21.021 | 39.200 | 9.634 | 1.00 | 17.00 |
| ATOM | 4086 | CG1 | ILE | B | 720 | 23.458 | 39.903 | 9.626 | 1.00 | 17.24 |
| ATOM | 4087 | CD1 | ILE | B | 720 | 23.165 | 41.216 | 8.938 | 1.00 | 18.75 |
| ATOM | 4088 | C | ILE | B | 720 | 21.725 | 36.484 | 10.083 | 1.00 | 16.85 |
| ATOM | 4089 | O | ILE | B | 720 | 20.966 | 36.339 | 11.044 | 1.00 | 17.13 |
| ATOM | 4090 | N | LEU | B | 721 | 21.628 | 35.741 | 8.983 | 1.00 | 17.26 |
| ATOM | 4091 | CA | LEU | B | 721 | 20.588 | 34.726 | 8.886 | 1.00 | 17.99 |
| ATOM | 4092 | CB | LEU | B | 721 | 20.611 | 34.056 | 7.507 | 1.00 | 21.38 |
| ATOM | 4093 | CG | LEU | B | 721 | 19.998 | 34.910 | 6.390 | 1.00 | 22.95 |
| ATOM | 4094 | CD1 | LEU | B | 721 | 20.102 | 34.181 | 5.071 | 1.00 | 22.96 |
| ATOM | 4095 | CD2 | LEU | B | 721 | 18.544 | 35.204 | 6.707 | 1.00 | 22.88 |
| ATOM | 4096 | C | LEU | B | 721 | 20.764 | 33.686 | 9.984 | 1.00 | 17.50 |
| ATOM | 4097 | O | LEU | B | 721 | 19.791 | 33.142 | 10.493 | 1.00 | 18.24 |
| ATOM | 4098 | N | ALA | B | 722 | 22.013 | 33.425 | 10.359 | 1.00 | 18.93 |
| ATOM | 4099 | CA | ALA | B | 722 | 22.305 | 32.448 | 11.398 | 1.00 | 19.70 |
| ATOM | 4100 | CB | ALA | B | 722 | 23.818 | 32.282 | 11.556 | 1.00 | 22.76 |
| ATOM | 4101 | C | ALA | B | 722 | 21.689 | 32.803 | 12.748 | 1.00 | 19.06 |
| ATOM | 4102 | O | ALA | B | 722 | 21.491 | 31.926 | 13.581 | 1.00 | 19.32 |
| ATOM | 4103 | N | THR | B | 723 | 21.389 | 34.081 | 12.972 | 1.00 | 19.12 |
| ATOM | 4104 | CA | THR | B | 723 | 20.820 | 34.484 | 14.255 | 1.00 | 18.93 |
| ATOM | 4105 | CB | THR | B | 723 | 20.997 | 36.000 | 14.503 | 1.00 | 16.46 |
| ATOM | 4106 | OG1 | THR | B | 723 | 20.359 | 36.746 | 13.463 | 1.00 | 18.08 |
| ATOM | 4107 | CG2 | THR | B | 723 | 22.485 | 36.355 | 14.536 | 1.00 | 17.70 |
| ATOM | 4108 | C | THR | B | 723 | 19.352 | 34.093 | 14.440 | 1.00 | 20.30 |
| ATOM | 4109 | O | THR | B | 723 | 18.744 | 34.373 | 15.474 | 1.00 | 18.88 |
| ATOM | 4110 | N | ASP | B | 724 | 18.785 | 33.464 | 13.421 | 1.00 | 20.75 |
| ATOM | 4111 | CA | ASP | B | 724 | 17.417 | 32.977 | 13.495 | 1.00 | 21.45 |
| ATOM | 4112 | CB | ASP | B | 724 | 16.901 | 32.670 | 12.084 | 1.00 | 20.47 |
| ATOM | 4113 | CG | ASP | B | 724 | 15.541 | 31.991 | 12.091 | 1.00 | 19.47 |
| ATOM | 4114 | OD1 | ASP | B | 724 | 14.982 | 31.769 | 13.183 | 1.00 | 20.37 |
| ATOM | 4115 | OD2 | ASP | B | 724 | 15.029 | 31.683 | 10.995 | 1.00 | 20.97 |
| ATOM | 4116 | C | ASP | B | 724 | 17.571 | 31.684 | 14.296 | 1.00 | 22.80 |
| ATOM | 4117 | O | ASP | B | 724 | 18.171 | 30.729 | 13.803 | 1.00 | 21.11 |
| ATOM | 4118 | N | LEU | B | 725 | 17.057 | 31.648 | 15.524 | 1.00 | 21.88 |
| ATOM | 4119 | CA | LEU | B | 725 | 17.211 | 30.454 | 16.354 | 1.00 | 23.26 |
| ATOM | 4120 | CB | LEU | B | 725 | 16.575 | 30.659 | 17.734 | 1.00 | 21.15 |
| ATOM | 4121 | CG | LEU | B | 725 | 17.439 | 31.482 | 18.698 | 1.00 | 22.29 |
| ATOM | 4122 | CD1 | LEU | B | 725 | 16.712 | 31.674 | 20.019 | 1.00 | 22.69 |
| ATOM | 4123 | CD2 | LEU | B | 725 | 18.768 | 30.765 | 18.927 | 1.00 | 22.08 |
| ATOM | 4124 | C | LEU | B | 725 | 16.685 | 29.178 | 15.720 | 1.00 | 24.43 |
| ATOM | 4125 | O | LEU | B | 725 | 17.084 | 28.074 | 16.110 | 1.00 | 24.43 |
| ATOM | 4126 | N | ALA | B | 726 | 15.802 | 29.322 | 14.735 | 1.00 | 24.46 |
| ATOM | 4127 | CA | ALA | B | 726 | 15.256 | 28.158 | 14.042 | 1.00 | 24.85 |
| ATOM | 4128 | CB | ALA | B | 726 | 14.096 | 28.576 | 13.144 | 1.00 | 26.10 |
| ATOM | 4129 | C | ALA | B | 726 | 16.361 | 27.502 | 13.219 | 1.00 | 25.58 |
| ATOM | 4130 | O | ALA | B | 726 | 16.436 | 26.277 | 13.127 | 1.00 | 24.81 |
| ATOM | 4131 | N | LEU | B | 727 | 17.217 | 28.323 | 12.617 | 1.00 | 25.66 |
| ATOM | 4132 | CA | LEU | B | 727 | 18.332 | 27.826 | 11.825 | 1.00 | 26.45 |
| ATOM | 4133 | CB | LEU | B | 727 | 18.952 | 28.969 | 11.016 | 1.00 | 32.52 |
| ATOM | 4134 | CG | LEU | B | 727 | 18.730 | 28.953 | 9.499 | 1.00 | 34.10 |
| ATOM | 4135 | CD1 | LEU | B | 727 | 17.320 | 28.490 | 9.163 | 1.00 | 32.76 |
| ATOM | 4136 | CD2 | LEU | B | 727 | 18.999 | 30.343 | 8.938 | 1.00 | 32.91 |
| ATOM | 4137 | C | LEU | B | 727 | 19.370 | 27.216 | 12.767 | 1.00 | 26.66 |
| ATOM | 4138 | O | LEU | B | 727 | 19.994 | 26.208 | 12.448 | 1.00 | 27.25 |
| ATOM | 4139 | N | TYR | B | 728 | 19.549 | 27.828 | 13.933 | 1.00 | 23.88 |
| ATOM | 4140 | CA | TYR | B | 728 | 20.497 | 27.311 | 14.912 | 1.00 | 24.96 |
| ATOM | 4141 | CB | TYR | B | 728 | 20.576 | 28.246 | 16.127 | 1.00 | 22.32 |
| ATOM | 4142 | CG | TYR | B | 728 | 21.197 | 27.606 | 17.348 | 1.00 | 22.57 |
| ATOM | 4143 | CD1 | TYR | B | 728 | 20.402 | 27.173 | 18.410 | 1.00 | 23.27 |
| ATOM | 4144 | CE1 | TYR | B | 728 | 20.958 | 26.520 | 19.512 | 1.00 | 22.36 |
| ATOM | 4145 | CD2 | TYR | B | 728 | 22.569 | 27.372 | 17.416 | 1.00 | 23.33 |
| ATOM | 4146 | CE2 | TYR | B | 728 | 23.133 | 26.713 | 18.512 | 1.00 | 22.71 |
| ATOM | 4147 | CZ | TYR | B | 728 | 22.322 | 26.292 | 19.553 | 1.00 | 23.34 |
| ATOM | 4148 | OH | TYR | B | 728 | 22.876 | 25.642 | 20.638 | 1.00 | 25.15 |
| ATOM | 4149 | C | TYR | B | 728 | 20.085 | 25.908 | 15.365 | 1.00 | 25.55 |
| ATOM | 4150 | O | TYR | B | 728 | 20.894 | 24.982 | 15.368 | 1.00 | 25.79 |
| ATOM | 4151 | N | ILE | B | 729 | 18.821 | 25.759 | 15.747 | 1.00 | 27.10 |
| ATOM | 4152 | CA | ILE | B | 729 | 18.313 | 24.476 | 16.218 | 1.00 | 27.90 |
| ATOM | 4153 | CB | ILE | B | 729 | 16.849 | 24.608 | 16.692 | 1.00 | 33.37 |
| ATOM | 4154 | CG2 | ILE | B | 729 | 16.245 | 23.231 | 16.936 | 1.00 | 34.11 |
| ATOM | 4155 | CG1 | ILE | B | 729 | 16.800 | 25.447 | 17.969 | 1.00 | 33.59 |
| ATOM | 4156 | CD1 | ILE | B | 729 | 15.400 | 25.662 | 18.512 | 1.00 | 34.44 |
| ATOM | 4157 | C | ILE | B | 729 | 18.396 | 23.395 | 15.151 | 1.00 | 28.43 |
| ATOM | 4158 | O | ILE | B | 729 | 18.561 | 22.212 | 15.463 | 1.00 | 28.49 |
| ATOM | 4159 | N | LYS | B | 730 | 18.286 | 23.803 | 13.893 | 1.00 | 30.10 |
| ATOM | 4160 | CA | LYS | B | 730 | 18.335 | 22.865 | 12.777 | 1.00 | 30.87 |
| ATOM | 4161 | CB | LYS | B | 730 | 17.637 | 23.474 | 11.554 | 1.00 | 41.47 |
| ATOM | 4162 | CG | LYS | B | 730 | 17.736 | 22.627 | 10.292 | 1.00 | 41.83 |
| ATOM | 4163 | CD | LYS | B | 730 | 17.082 | 23.312 | 9.099 | 1.00 | 41.85 |
| ATOM | 4164 | CE | LYS | B | 730 | 17.161 | 22.437 | 7.856 | 1.00 | 42.29 |
| ATOM | 4165 | NZ | LYS | B | 730 | 16.471 | 23.056 | 6.689 | 1.00 | 41.92 |
| ATOM | 4166 | C | LYS | B | 730 | 19.753 | 22.450 | 12.390 | 1.00 | 30.30 |
| ATOM | 4167 | O | LYS | B | 730 | 19.968 | 21.341 | 11.899 | 1.00 | 30.78 |
| ATOM | 4168 | N | ARG | B | 731 | 20.722 | 23.330 | 12.611 | 1.00 | 26.44 |
| ATOM | 4169 | CA | ARG | B | 731 | 22.101 | 23.029 | 12.234 | 1.00 | 26.05 |
| ATOM | 4170 | CB | ARG | B | 731 | 22.691 | 24.207 | 11.455 | 1.00 | 44.87 |
| ATOM | 4171 | CG | ARG | B | 731 | 22.027 | 24.477 | 10.123 | 1.00 | 45.35 |
| ATOM | 4172 | CD | ARG | B | 731 | 22.579 | 25.739 | 9.481 | 1.00 | 45.70 |
| ATOM | 4173 | NE | ARG | B | 731 | 24.018 | 25.666 | 9.236 | 1.00 | 45.72 |
| ATOM | 4174 | CZ | ARG | B | 731 | 24.600 | 24.771 | 8.445 | 1.00 | 45.64 |
| ATOM | 4175 | NH1 | ARG | B | 731 | 23.870 | 23.859 | 7.816 | 1.00 | 45.97 |
| ATOM | 4176 | NH2 | ARG | B | 731 | 25.915 | 24.793 | 8.272 | 1.00 | 45.83 |
| ATOM | 4177 | C | ARG | B | 731 | 23.076 | 22.657 | 13.349 | 1.00 | 25.87 |
| ATOM | 4178 | O | ARG | B | 731 | 24.144 | 22.121 | 13.069 | 1.00 | 25.60 |
| ATOM | 4179 | N | ARG | B | 732 | 22.724 | 22.931 | 14.600 | 1.00 | 24.75 |
| ATOM | 4180 | CA | ARG | B | 732 | 23.636 | 22.646 | 15.704 | 1.00 | 24.59 |
| ATOM | 4181 | CB | ARG | B | 732 | 23.051 | 23.154 | 17.021 | 1.00 | 28.71 |
| ATOM | 4182 | CG | ARG | B | 732 | 21.825 | 22.412 | 17.490 | 1.00 | 28.36 |
| ATOM | 4183 | CD | ARG | B | 732 | 21.266 | 23.052 | 18.742 | 1.00 | 28.99 |
| ATOM | 4184 | NE | ARG | B | 732 | 20.160 | 22.282 | 19.295 | 1.00 | 28.09 |
| ATOM | 4185 | CZ | ARG | B | 732 | 19.555 | 22.567 | 20.444 | 1.00 | 29.06 |
| ATOM | 4186 | NH1 | ARG | B | 732 | 19.951 | 23.607 | 21.164 | 1.00 | 29.87 |
| ATOM | 4187 | NH2 | ARG | B | 732 | 18.560 | 21.807 | 20.878 | 1.00 | 29.81 |
| ATOM | 4188 | C | ARG | B | 732 | 24.027 | 21.177 | 15.845 | 1.00 | 24.59 |
| ATOM | 4189 | O | ARG | B | 732 | 25.143 | 20.871 | 16.272 | 1.00 | 24.56 |
| ATOM | 4190 | N | GLY | B | 733 | 23.114 | 20.278 | 15.495 | 1.00 | 28.28 |
| ATOM | 4191 | CA | GLY | B | 733 | 23.409 | 18.857 | 15.592 | 1.00 | 29.55 |
| ATOM | 4192 | C | GLY | B | 733 | 24.686 | 18.506 | 14.852 | 1.00 | 29.73 |
| ATOM | 4193 | O | GLY | B | 733 | 25.582 | 17.856 | 15.402 | 1.00 | 29.54 |
| ATOM | 4194 | N | GLU | B | 734 | 24.775 | 18.940 | 13.599 | 1.00 | 29.63 |
| ATOM | 4195 | CA | GLU | B | 734 | 25.954 | 18.676 | 12.779 | 1.00 | 29.74 |
| ATOM | 4196 | CB | GLU | B | 734 | 25.772 | 19.317 | 11.396 | 1.00 | 37.09 |
| ATOM | 4197 | CG | GLU | B | 734 | 27.011 | 19.313 | 10.513 | 1.00 | 37.69 |
| ATOM | 4198 | CD | GLU | B | 734 | 26.712 | 19.749 | 9.090 | 1.00 | 37.69 |
| ATOM | 4199 | OE1 | GLU | B | 734 | 25.895 | 20.673 | 8.905 | 1.00 | 37.77 |
| ATOM | 4200 | OE2 | GLU | B | 734 | 27.302 | 19.174 | 8.153 | 1.00 | 37.96 |
| ATOM | 4201 | C | GLU | B | 734 | 27.234 | 19.187 | 13.448 | 1.00 | 29.89 |
| ATOM | 4202 | O | GLU | B | 734 | 28.241 | 18.481 | 13.508 | 1.00 | 29.99 |
| ATOM | 4203 | N | PHE | B | 735 | 27.193 | 20.416 | 13.953 | 1.00 | 26.19 |
| ATOM | 4204 | CA | PHE | B | 735 | 28.348 | 21.015 | 14.617 | 1.00 | 28.01 |
| ATOM | 4205 | CB | PHE | B | 735 | 28.001 | 22.449 | 15.051 | 1.00 | 32.46 |
| ATOM | 4206 | CG | PHE | B | 735 | 29.121 | 23.170 | 15.748 | 1.00 | 32.58 |
| ATOM | 4207 | CD1 | PHE | B | 735 | 30.350 | 23.353 | 15.121 | 1.00 | 32.98 |
| ATOM | 4208 | CD2 | PHE | B | 735 | 28.930 | 23.710 | 17.018 | 1.00 | 32.86 |
| ATOM | 4209 | CE1 | PHE | B | 735 | 31.374 | 24.067 | 15.746 | 1.00 | 33.43 |
| ATOM | 4210 | CE2 | PHE | B | 735 | 29.941 | 24.424 | 17.653 | 1.00 | 33.17 |
| ATOM | 4211 | CZ | PHE | B | 735 | 31.168 | 24.606 | 17.017 | 1.00 | 33.32 |
| ATOM | 4212 | C | PHE | B | 735 | 28.759 | 20.185 | 15.832 | 1.00 | 28.38 |
| ATOM | 4213 | O | PHE | B | 735 | 29.943 | 19.877 | 16.023 | 1.00 | 28.13 |
| ATOM | 4214 | N | PHE | B | 736 | 27.772 | 19.823 | 16.646 | 1.00 | 35.56 |
| ATOM | 4215 | CA | PHE | B | 736 | 28.008 | 19.033 | 17.848 | 1.00 | 36.50 |
| ATOM | 4216 | CB | PHE | B | 736 | 26.695 | 18.843 | 18.615 | 1.00 | 29.57 |
| ATOM | 4217 | CG | PHE | B | 736 | 26.153 | 20.118 | 19.217 | 1.00 | 28.48 |
| ATOM | 4218 | CD1 | PHE | B | 736 | 24.849 | 20.174 | 19.707 | 1.00 | 29.25 |
| ATOM | 4219 | CD2 | PHE | B | 736 | 26.947 | 21.261 | 19.298 | 1.00 | 28.68 |
| ATOM | 4220 | CE1 | PHE | B | 736 | 24.343 | 21.351 | 20.270 | 1.00 | 28.48 |
| ATOM | 4221 | CE2 | PHE | B | 736 | 26.451 | 22.440 | 19.857 | 1.00 | 28.64 |
| ATOM | 4222 | CZ | PHE | B | 736 | 25.142 | 22.481 | 20.344 | 1.00 | 28.27 |
| ATOM | 4223 | C | PHE | B | 736 | 28.636 | 17.678 | 17.538 | 1.00 | 36.98 |
| ATOM | 4224 | O | PHE | B | 736 | 29.472 | 17.192 | 18.299 | 1.00 | 37.51 |
| ATOM | 4225 | N | GLU | B | 737 | 28.236 | 17.074 | 16.422 | 1.00 | 35.33 |
| ATOM | 4226 | CA | GLU | B | 737 | 28.779 | 15.778 | 16.019 | 1.00 | 34.66 |
| ATOM | 4227 | CB | GLU | B | 737 | 28.042 | 15.245 | 14.788 | 1.00 | 48.23 |
| ATOM | 4228 | CG | GLU | B | 737 | 26.624 | 14.782 | 15.052 | 1.00 | 48.59 |
| ATOM | 4229 | CD | GLU | B | 737 | 26.562 | 13.677 | 16.084 | 1.00 | 48.98 |
| ATOM | 4230 | OE1 | GLU | B | 737 | 27.347 | 12.710 | 15.969 | 1.00 | 48.80 |
| ATOM | 4231 | OE2 | GLU | B | 737 | 25.724 | 13.771 | 17.004 | 1.00 | 49.04 |
| ATOM | 4232 | C | GLU | B | 737 | 30.264 | 15.895 | 15.698 | 1.00 | 35.25 |
| ATOM | 4233 | O | GLU | B | 737 | 31.095 | 15.184 | 16.276 | 1.00 | 34.50 |
| ATOM | 4234 | N | LEU | B | 738 | 30.582 | 16.792 | 14.766 | 1.00 | 34.53 |
| ATOM | 4235 | CA | LEU | B | 738 | 31.958 | 17.036 | 14.339 | 1.00 | 34.99 |
| ATOM | 4236 | CB | LEU | B | 738 | 32.010 | 18.266 | 13.422 | 1.00 | 33.43 |
| ATOM | 4237 | CG | LEU | B | 738 | 31.532 | 18.137 | 11.969 | 1.00 | 33.44 |
| ATOM | 4238 | CD1 | LEU | B | 738 | 30.237 | 17.345 | 11.885 | 1.00 | 33.77 |
| ATOM | 4239 | CD2 | LEU | B | 738 | 31.345 | 19.531 | 11.388 | 1.00 | 33.47 |
| ATOM | 4240 | C | LEU | B | 738 | 32.889 | 17.243 | 15.530 | 1.00 | 35.52 |
| ATOM | 4241 | O | LEU | B | 738 | 34.020 | 16.760 | 15.535 | 1.00 | 35.19 |
| ATOM | 4242 | N | ILE | B | 739 | 32.403 | 17.960 | 16.538 | 1.00 | 35.96 |
| ATOM | 4243 | CA | ILE | B | 739 | 33.177 | 18.243 | 17.740 | 1.00 | 36.59 |
| ATOM | 4244 | CB | ILE | B | 739 | 32.489 | 19.347 | 18.578 | 1.00 | 41.13 |
| ATOM | 4245 | CG2 | ILE | B | 739 | 33.192 | 19.509 | 19.913 | 1.00 | 41.32 |
| ATOM | 4246 | CG1 | ILE | B | 739 | 32.478 | 20.665 | 17.798 | 1.00 | 41.45 |
| ATOM | 4247 | CD1 | ILE | B | 739 | 33.852 | 21.224 | 17.510 | 1.00 | 41.21 |
| ATOM | 4248 | C | ILE | B | 739 | 33.353 | 16.998 | 18.610 | 1.00 | 37.53 |
| ATOM | 4249 | O | ILE | B | 739 | 34.455 | 16.711 | 19.088 | 1.00 | 36.85 |
| ATOM | 4250 | N | ARG | B | 740 | 32.262 | 16.264 | 18.808 | 1.00 | 40.80 |
| ATOM | 4251 | CA | ARG | B | 740 | 32.276 | 15.058 | 19.627 | 1.00 | 41.57 |
| ATOM | 4252 | CB | ARG | B | 740 | 30.860 | 14.490 | 19.744 | 1.00 | 49.23 |
| ATOM | 4253 | CG | ARG | B | 740 | 30.708 | 13.372 | 20.763 | 1.00 | 49.50 |
| ATOM | 4254 | CD | ARG | B | 740 | 29.268 | 12.894 | 20.816 | 1.00 | 49.76 |
| ATOM | 4255 | NE | ARG | B | 740 | 28.870 | 12.228 | 19.577 | 1.00 | 49.63 |
| ATOM | 4256 | CZ | ARG | B | 740 | 29.192 | 10.977 | 19.264 | 1.00 | 49.97 |
| ATOM | 4257 | NH1 | ARG | B | 740 | 29.917 | 10.246 | 20.103 | 1.00 | 49.93 |
| ATOM | 4258 | NH2 | ARG | B | 740 | 28.795 | 10.456 | 18.111 | 1.00 | 49.81 |
| ATOM | 4259 | C | ARG | B | 740 | 33.202 | 13.999 | 19.045 | 1.00 | 41.31 |
| ATOM | 4260 | O | ARG | B | 740 | 34.041 | 13.437 | 19.750 | 1.00 | 41.75 |
| ATOM | 4261 | N | LYS | B | 741 | 33.048 | 13.734 | 17.753 | 1.00 | 36.01 |
| ATOM | 4262 | CA | LYS | B | 741 | 33.858 | 12.734 | 17.074 | 1.00 | 36.04 |
| ATOM | 4263 | CB | LYS | B | 741 | 33.142 | 12.261 | 15.806 | 1.00 | 51.86 |
| ATOM | 4264 | CG | LYS | B | 741 | 31.823 | 11.547 | 16.084 | 1.00 | 52.33 |
| ATOM | 4265 | CD | LYS | B | 741 | 31.198 | 10.984 | 14.819 | 1.00 | 52.60 |
| ATOM | 4266 | CE | LYS | B | 741 | 29.956 | 10.164 | 15.147 | 1.00 | 52.72 |
| ATOM | 4267 | NZ | LYS | B | 741 | 29.371 | 9.514 | 13.941 | 1.00 | 52.84 |
| ATOM | 4268 | C | LYS | B | 741 | 35.257 | 13.229 | 16.739 | 1.00 | 36.83 |
| ATOM | 4269 | O | LYS | B | 741 | 36.017 | 12.544 | 16.056 | 1.00 | 35.76 |
| ATOM | 4270 | N | ASN | B | 742 | 35.598 | 14.424 | 17.217 | 1.00 | 47.96 |
| ATOM | 4271 | CA | ASN | B | 742 | 36.925 | 14.991 | 16.979 | 1.00 | 48.89 |
| ATOM | 4272 | CB | ASN | B | 742 | 37.984 | 14.073 | 17.601 | 1.00 | 45.18 |
| ATOM | 4273 | CG | ASN | B | 742 | 37.660 | 13.696 | 19.038 | 1.00 | 45.25 |
| ATOM | 4274 | OD1 | ASN | B | 742 | 37.873 | 14.481 | 19.958 | 1.00 | 45.19 |
| ATOM | 4275 | ND2 | ASN | B | 742 | 37.127 | 12.492 | 19.231 | 1.00 | 45.61 |
| ATOM | 4276 | C | ASN | B | 742 | 37.210 | 15.160 | 15.482 | 1.00 | 48.30 |
| ATOM | 4277 | O | ASN | B | 742 | 38.318 | 14.886 | 15.023 | 1.00 | 49.56 |
| ATOM | 4278 | N | GLN | B | 743 | 36.214 | 15.627 | 14.733 | 1.00 | 35.63 |
| ATOM | 4279 | CA | GLN | B | 743 | 36.345 | 15.806 | 13.288 | 1.00 | 33.02 |
| ATOM | 4280 | CB | GLN | B | 743 | 35.226 | 15.034 | 12.583 | 1.00 | 55.29 |
| ATOM | 4281 | CG | GLN | B | 743 | 34.914 | 13.679 | 13.206 | 1.00 | 55.90 |
| ATOM | 4282 | CD | GLN | B | 743 | 33.531 | 13.159 | 12.830 | 1.00 | 56.30 |
| ATOM | 4283 | OE1 | GLN | B | 743 | 32.531 | 13.872 | 12.943 | 1.00 | 56.03 |
| ATOM | 4284 | NE2 | GLN | B | 743 | 33.470 | 11.903 | 12.396 | 1.00 | 56.30 |
| ATOM | 4285 | C | GLN | B | 743 | 36.296 | 17.272 | 12.833 | 1.00 | 32.63 |
| ATOM | 4286 | O | GLN | B | 743 | 36.519 | 17.568 | 11.659 | 1.00 | 30.98 |
| ATOM | 4287 | N | PHE | B | 744 | 36.008 | 18.186 | 13.754 | 1.00 | 30.07 |
| ATOM | 4288 | CA | PHE | B | 744 | 35.913 | 19.600 | 13.396 | 1.00 | 29.86 |
| ATOM | 4289 | CB | PHE | B | 744 | 35.664 | 20.451 | 14.646 | 1.00 | 36.04 |
| ATOM | 4290 | CG | PHE | B | 744 | 35.433 | 21.906 | 14.349 | 1.00 | 36.02 |
| ATOM | 4291 | CD1 | PHE | B | 744 | 34.426 | 22.294 | 13.472 | 1.00 | 36.51 |
| ATOM | 4292 | CD2 | PHE | B | 744 | 36.224 | 22.886 | 14.939 | 1.00 | 36.37 |
| ATOM | 4293 | CE1 | PHE | B | 744 | 34.210 | 23.641 | 13.186 | 1.00 | 36.33 |
| ATOM | 4294 | CE2 | PHE | B | 744 | 36.017 | 24.237 | 14.660 | 1.00 | 36.35 |
| ATOM | 4295 | CZ | PHE | B | 744 | 35.007 | 24.613 | 13.781 | 1.00 | 36.44 |
| ATOM | 4296 | C | PHE | B | 744 | 37.158 | 20.101 | 12.671 | 1.00 | 30.17 |
| ATOM | 4297 | O | PHE | B | 744 | 38.274 | 19.948 | 13.163 | 1.00 | 29.71 |
| ATOM | 4298 | N | ASN | B | 745 | 36.953 | 20.703 | 11.500 | 1.00 | 33.40 |
| ATOM | 4299 | CA | ASN | B | 745 | 38.047 | 21.229 | 10.685 | 1.00 | 34.97 |
| ATOM | 4300 | CB | ASN | B | 745 | 38.463 | 20.192 | 9.634 | 1.00 | 36.63 |
| ATOM | 4301 | CG | ASN | B | 745 | 39.397 | 20.765 | 8.586 | 1.00 | 36.73 |
| ATOM | 4302 | OD1 | ASN | B | 745 | 40.281 | 21.562 | 8.896 | 1.00 | 37.02 |
| ATOM | 4303 | ND2 | ASN | B | 745 | 39.216 | 20.345 | 7.337 | 1.00 | 37.05 |
| ATOM | 4304 | C | ASN | B | 745 | 37.674 | 22.537 | 9.992 | 1.00 | 34.71 |
| ATOM | 4305 | O | ASN | B | 745 | 36.930 | 22.540 | 9.011 | 1.00 | 34.81 |
| ATOM | 4306 | N | LEU | B | 746 | 38.209 | 23.645 | 10.495 | 1.00 | 32.32 |
| ATOM | 4307 | CA | LEU | B | 746 | 37.914 | 24.961 | 9.934 | 1.00 | 31.51 |
| ATOM | 4308 | CB | LEU | B | 746 | 38.459 | 26.059 | 10.853 | 1.00 | 35.95 |
| ATOM | 4309 | CG | LEU | B | 746 | 37.591 | 26.325 | 12.083 | 1.00 | 36.41 |
| ATOM | 4310 | CD1 | LEU | B | 746 | 38.239 | 27.390 | 12.951 | 1.00 | 36.26 |
| ATOM | 4311 | CD2 | LEU | B | 746 | 36.200 | 26.773 | 11.635 | 1.00 | 36.10 |
| ATOM | 4312 | C | LEU | B | 746 | 38.419 | 25.181 | 8.511 | 1.00 | 31.36 |
| ATOM | 4313 | O | LEU | B | 746 | 38.085 | 26.183 | 7.877 | 1.00 | 31.04 |
| ATOM | 4314 | N | GLU | B | 747 | 39.224 | 24.252 | 8.010 | 1.00 | 30.95 |
| ATOM | 4315 | CA | GLU | B | 747 | 39.738 | 24.357 | 6.650 | 1.00 | 31.86 |
| ATOM | 4316 | CB | GLU | B | 747 | 40.918 | 23.402 | 6.453 | 1.00 | 51.07 |
| ATOM | 4317 | CG | GLU | B | 747 | 42.129 | 23.738 | 7.311 | 1.00 | 51.73 |
| ATOM | 4318 | CD | GLU | B | 747 | 43.251 | 22.728 | 7.163 | 1.00 | 52.11 |
| ATOM | 4319 | OE1 | GLU | B | 747 | 43.666 | 22.467 | 6.014 | 1.00 | 52.03 |
| ATOM | 4320 | OE2 | GLU | B | 747 | 43.718 | 22.201 | 8.196 | 1.00 | 52.21 |
| ATOM | 4321 | C | GLU | B | 747 | 38.617 | 24.014 | 5.670 | 1.00 | 31.25 |
| ATOM | 4322 | O | GLU | B | 747 | 38.646 | 24.421 | 4.507 | 1.00 | 30.95 |
| ATOM | 4323 | N | ASP | B | 748 | 37.634 | 23.260 | 6.156 | 1.00 | 31.79 |
| ATOM | 4324 | CA | ASP | B | 748 | 36.480 | 22.864 | 5.357 | 1.00 | 31.93 |
| ATOM | 4325 | CB | ASP | B | 748 | 35.754 | 21.697 | 6.031 | 1.00 | 39.97 |
| ATOM | 4326 | CG | ASP | B | 748 | 34.571 | 21.193 | 5.223 | 1.00 | 40.51 |
| ATOM | 4327 | OD1 | ASP | B | 748 | 33.678 | 21.995 | 4.889 | 1.00 | 40.27 |
| ATOM | 4328 | OD2 | ASP | B | 748 | 34.533 | 19.981 | 4.928 | 1.00 | 40.36 |
| ATOM | 4329 | C | ASP | B | 748 | 35.546 | 24.071 | 5.265 | 1.00 | 32.12 |
| ATOM | 4330 | O | ASP | B | 748 | 35.026 | 24.538 | 6.280 | 1.00 | 31.68 |
| ATOM | 4331 | N | PRO | B | 749 | 35.317 | 24.586 | 4.046 | 1.00 | 30.33 |
| ATOM | 4332 | CD | PRO | B | 749 | 35.694 | 23.972 | 2.761 | 1.00 | 44.68 |
| ATOM | 4333 | CA | PRO | B | 749 | 34.445 | 25.746 | 3.827 | 1.00 | 30.40 |
| ATOM | 4334 | CB | PRO | B | 749 | 34.380 | 25.845 | 2.304 | 1.00 | 44.80 |
| ATOM | 4335 | CG | PRO | B | 749 | 34.577 | 24.423 | 1.863 | 1.00 | 45.23 |
| ATOM | 4336 | C | PRO | B | 749 | 33.065 | 25.626 | 4.470 | 1.00 | 30.78 |
| ATOM | 4337 | O | PRO | B | 749 | 32.553 | 26.591 | 5.045 | 1.00 | 29.64 |
| ATOM | 4338 | N | HIS | B | 750 | 32.464 | 24.443 | 4.379 | 1.00 | 31.42 |
| ATOM | 4339 | CA | HIS | B | 750 | 31.148 | 24.231 | 4.963 | 1.00 | 31.75 |
| ATOM | 4340 | CB | HIS | B | 750 | 30.569 | 22.890 | 4.513 | 1.00 | 36.05 |
| ATOM | 4341 | CG | HIS | B | 750 | 29.289 | 22.529 | 5.200 | 1.00 | 36.27 |
| ATOM | 4342 | CD2 | HIS | B | 750 | 28.002 | 22.836 | 4.908 | 1.00 | 35.87 |
| ATOM | 4343 | ND1 | HIS | B | 750 | 29.250 | 21.786 | 6.360 | 1.00 | 36.16 |
| ATOM | 4344 | CE1 | HIS | B | 750 | 27.996 | 21.648 | 6.752 | 1.00 | 36.46 |
| ATOM | 4345 | NE2 | HIS | B | 750 | 27.219 | 22.276 | 5.888 | 1.00 | 36.37 |
| ATOM | 4346 | C | HIS | B | 750 | 31.212 | 24.288 | 6.480 | 1.00 | 31.05 |
| ATOM | 4347 | O | HIS | B | 750 | 30.373 | 24.922 | 7.118 | 1.00 | 31.52 |
| ATOM | 4348 | N | GLU | B | 751 | 32.208 | 23.629 | 7.062 | 1.00 | 26.46 |
| ATOM | 4349 | CA | GLU | B | 751 | 32.356 | 23.642 | 8.512 | 1.00 | 26.08 |
| ATOM | 4350 | CB | GLU | B | 751 | 33.415 | 22.625 | 8.946 | 1.00 | 32.16 |
| ATOM | 4351 | CG | GLU | B | 751 | 33.004 | 21.185 | 8.700 | 1.00 | 32.81 |
| ATOM | 4352 | CD | GLU | B | 751 | 34.040 | 20.193 | 9.190 | 1.00 | 32.54 |
| ATOM | 4353 | OE1 | GLU | B | 751 | 34.381 | 20.234 | 10.392 | 1.00 | 32.25 |
| ATOM | 4354 | OE2 | GLU | B | 751 | 34.507 | 19.373 | 8.372 | 1.00 | 33.04 |
| ATOM | 4355 | C | GLU | B | 751 | 32.741 | 25.038 | 8.997 | 1.00 | 25.23 |
| ATOM | 4356 | O | GLU | B | 751 | 32.426 | 25.424 | 10.127 | 1.00 | 26.00 |
| ATOM | 4357 | N | LYS | B | 752 | 33.413 | 25.791 | 8.134 | 1.00 | 25.46 |
| ATOM | 4358 | CA | LYS | B | 752 | 33.835 | 27.144 | 8.477 | 1.00 | 25.14 |
| ATOM | 4359 | CB | LYS | B | 752 | 34.760 | 27.710 | 7.400 | 1.00 | 34.00 |
| ATOM | 4360 | CG | LYS | B | 752 | 35.473 | 28.988 | 7.807 | 1.00 | 34.35 |
| ATOM | 4361 | CD | LYS | B | 752 | 36.445 | 28.715 | 8.950 | 1.00 | 34.98 |
| ATOM | 4362 | CE | LYS | B | 752 | 37.386 | 29.878 | 9.166 | 1.00 | 34.20 |
| ATOM | 4363 | NZ | LYS | B | 752 | 38.098 | 30.208 | 7.910 | 1.00 | 34.22 |
| ATOM | 4364 | C | LYS | B | 752 | 32.600 | 28.024 | 8.593 | 1.00 | 25.44 |
| ATOM | 4365 | O | LYS | B | 752 | 32.435 | 28.747 | 9.574 | 1.00 | 25.14 |
| ATOM | 4366 | N | GLU | B | 753 | 31.724 | 27.956 | 7.597 | 1.00 | 26.17 |
| ATOM | 4367 | CA | GLU | B | 753 | 30.519 | 28.775 | 7.639 | 1.00 | 27.32 |
| ATOM | 4368 | CB | GLU | B | 753 | 29.773 | 28.743 | 6.300 | 1.00 | 47.16 |
| ATOM | 4369 | CG | GLU | B | 753 | 29.010 | 27.468 | 6.008 | 1.00 | 48.02 |
| ATOM | 4370 | CD | GLU | B | 753 | 28.190 | 27.578 | 4.738 | 1.00 | 48.29 |
| ATOM | 4371 | OE1 | GLU | B | 753 | 28.791 | 27.692 | 3.647 | 1.00 | 48.21 |
| ATOM | 4372 | OE2 | GLU | B | 753 | 26.944 | 27.561 | 4.832 | 1.00 | 48.25 |
| ATOM | 4373 | C | GLU | B | 753 | 29.620 | 28.287 | 8.764 | 1.00 | 26.46 |
| ATOM | 4374 | O | GLU | B | 753 | 28.913 | 29.080 | 9.383 | 1.00 | 26.43 |
| ATOM | 4375 | N | LEU | B | 754 | 29.658 | 26.983 | 9.035 | 1.00 | 24.18 |
| ATOM | 4376 | CA | LEU | B | 754 | 28.860 | 26.406 | 10.110 | 1.00 | 22.29 |
| ATOM | 4377 | CB | LEU | B | 754 | 28.981 | 24.877 | 10.116 | 1.00 | 25.44 |
| ATOM | 4378 | CG | LEU | B | 754 | 28.264 | 24.173 | 11.273 | 1.00 | 25.42 |
| ATOM | 4379 | CD1 | LEU | B | 754 | 26.772 | 24.485 | 11.234 | 1.00 | 26.03 |
| ATOM | 4380 | CD2 | LEU | B | 754 | 28.494 | 22.674 | 11.174 | 1.00 | 26.23 |
| ATOM | 4381 | C | LEU | B | 754 | 29.338 | 26.957 | 11.451 | 1.00 | 22.78 |
| ATOM | 4382 | O | LEU | B | 754 | 28.524 | 27.323 | 12.308 | 1.00 | 23.06 |
| ATOM | 4383 | N | PHE | B | 755 | 30.657 | 27.015 | 11.636 | 1.00 | 20.22 |
| ATOM | 4384 | CA | PHE | B | 755 | 31.216 | 27.528 | 12.872 | 1.00 | 20.52 |
| ATOM | 4385 | CB | PHE | B | 755 | 32.745 | 27.445 | 12.870 | 1.00 | 21.28 |
| ATOM | 4386 | CG | PHE | B | 755 | 33.380 | 28.142 | 14.042 | 1.00 | 20.97 |
| ATOM | 4387 | CD1 | PHE | B | 755 | 33.212 | 27.654 | 15.332 | 1.00 | 21.53 |
| ATOM | 4388 | CD2 | PHE | B | 755 | 34.111 | 29.309 | 13.858 | 1.00 | 22.19 |
| ATOM | 4389 | CE1 | PHE | B | 755 | 33.763 | 28.323 | 16.426 | 1.00 | 21.26 |
| ATOM | 4390 | CE2 | PHE | B | 755 | 34.667 | 29.989 | 14.949 | 1.00 | 21.93 |
| ATOM | 4391 | CZ | PHE | B | 755 | 34.492 | 29.496 | 16.230 | 1.00 | 21.53 |
| ATOM | 4392 | C | PHE | B | 755 | 30.811 | 28.982 | 13.064 | 1.00 | 19.98 |
| ATOM | 4393 | O | PHE | B | 755 | 30.378 | 29.372 | 14.147 | 1.00 | 22.32 |
| ATOM | 4394 | N | LEU | B | 756 | 30.968 | 29.778 | 12.010 | 1.00 | 19.82 |
| ATOM | 4395 | CA | LEU | B | 756 | 30.621 | 31.193 | 12.070 | 1.00 | 19.75 |
| ATOM | 4396 | CB | LEU | B | 756 | 30.868 | 31.858 | 10.716 | 1.00 | 18.84 |
| ATOM | 4397 | CG | LEU | B | 756 | 32.329 | 31.854 | 10.252 | 1.00 | 18.79 |
| ATOM | 4398 | CD1 | LEU | B | 756 | 32.433 | 32.482 | 8.864 | 1.00 | 19.24 |
| ATOM | 4399 | CD2 | LEU | B | 756 | 33.192 | 32.610 | 11.262 | 1.00 | 19.62 |
| ATOM | 4400 | C | LEU | B | 756 | 29.165 | 31.372 | 12.488 | 1.00 | 21.11 |
| ATOM | 4401 | O | LEU | B | 756 | 28.839 | 32.294 | 13.233 | 1.00 | 19.94 |
| ATOM | 4402 | N | ALA | B | 757 | 28.291 | 30.488 | 12.014 | 1.00 | 20.80 |
| ATOM | 4403 | CA | ALA | B | 757 | 26.873 | 30.568 | 12.373 | 1.00 | 21.25 |
| ATOM | 4404 | CB | ALA | B | 757 | 26.064 | 29.560 | 11.552 | 1.00 | 23.40 |
| ATOM | 4405 | C | ALA | B | 757 | 26.693 | 30.294 | 13.861 | 1.00 | 22.25 |
| ATOM | 4406 | O | ALA | B | 757 | 25.917 | 30.971 | 14.542 | 1.00 | 22.17 |
| ATOM | 4407 | N | MET | B | 758 | 27.412 | 29.291 | 14.364 | 1.00 | 19.96 |
| ATOM | 4408 | CA | MET | B | 758 | 27.348 | 28.926 | 15.771 | 1.00 | 20.73 |
| ATOM | 4409 | CB | MET | B | 758 | 28.154 | 27.647 | 16.023 | 1.00 | 27.97 |
| ATOM | 4410 | CG | MET | B | 758 | 27.601 | 26.422 | 15.324 | 1.00 | 27.07 |
| ATOM | 4411 | SD | MET | B | 758 | 25.940 | 25.980 | 15.881 | 1.00 | 27.12 |
| ATOM | 4412 | CE | MET | B | 758 | 24.971 | 26.627 | 14.530 | 1.00 | 27.43 |
| ATOM | 4413 | C | MET | B | 758 | 27.895 | 30.043 | 16.656 | 1.00 | 18.82 |
| ATOM | 4414 | O | MET | B | 758 | 27.352 | 30.315 | 17.725 | 1.00 | 21.47 |
| ATOM | 4415 | N | LEU | B | 759 | 28.976 | 30.674 | 16.208 | 1.00 | 19.71 |
| ATOM | 4416 | CA | LEU | B | 759 | 29.597 | 31.756 | 16.954 | 1.00 | 18.16 |
| ATOM | 4417 | CB | LEU | B | 759 | 30.892 | 32.189 | 16.270 | 1.00 | 20.33 |
| ATOM | 4418 | CG | LEU | B | 759 | 31.646 | 33.349 | 16.919 | 1.00 | 19.85 |
| ATOM | 4419 | CD1 | LEU | B | 759 | 31.992 | 33.005 | 18.363 | 1.00 | 21.09 |
| ATOM | 4420 | CD2 | LEU | B | 759 | 32.891 | 33.646 | 16.103 | 1.00 | 19.80 |
| ATOM | 4421 | C | LEU | B | 759 | 28.635 | 32.933 | 17.048 | 1.00 | 20.05 |
| ATOM | 4422 | O | LEU | B | 759 | 28.535 | 33.577 | 18.091 | 1.00 | 19.37 |
| ATOM | 4423 | N | MET | B | 760 | 27.921 | 33.202 | 15.960 | 1.00 | 18.13 |
| ATOM | 4424 | CA | MET | B | 760 | 26.955 | 34.302 | 15.959 | 1.00 | 19.70 |
| ATOM | 4425 | CB | MET | B | 760 | 26.325 | 34.455 | 14.575 | 1.00 | 17.26 |
| ATOM | 4426 | CG | MET | B | 760 | 27.211 | 35.177 | 13.547 | 1.00 | 16.88 |
| ATOM | 4427 | SD | MET | B | 760 | 27.567 | 36.936 | 13.895 | 1.00 | 17.69 |
| ATOM | 4428 | CE | MET | B | 760 | 25.887 | 37.644 | 13.962 | 1.00 | 17.02 |
| ATOM | 4429 | C | MET | B | 760 | 25.879 | 34.059 | 17.017 | 1.00 | 18.46 |
| ATOM | 4430 | O | MET | B | 760 | 25.510 | 34.970 | 17.765 | 1.00 | 18.74 |
| ATOM | 4431 | N | THR | B | 761 | 25.380 | 32.831 | 17.102 | 1.00 | 17.88 |
| ATOM | 4432 | CA | THR | B | 761 | 24.358 | 32.516 | 18.092 | 1.00 | 17.90 |
| ATOM | 4433 | CB | THR | B | 761 | 23.858 | 31.070 | 17.933 | 1.00 | 20.06 |
| ATOM | 4434 | OG1 | THR | B | 761 | 23.316 | 30.905 | 16.619 | 1.00 | 20.01 |
| ATOM | 4435 | CG2 | THR | B | 761 | 22.778 | 30.746 | 18.971 | 1.00 | 19.13 |
| ATOM | 4436 | C | THR | B | 761 | 24.922 | 32.694 | 19.495 | 1.00 | 18.35 |
| ATOM | 4437 | O | THR | B | 761 | 24.265 | 33.241 | 20.375 | 1.00 | 17.84 |
| ATOM | 4438 | N | ALA | B | 762 | 26.160 | 32.244 | 19.695 | 1.00 | 18.42 |
| ATOM | 4439 | CA | ALA | B | 762 | 26.808 | 32.347 | 20.996 | 1.00 | 17.98 |
| ATOM | 4440 | CB | ALA | B | 762 | 28.224 | 31.780 | 20.930 | 1.00 | 19.48 |
| ATOM | 4441 | C | ALA | B | 762 | 26.843 | 33.795 | 21.472 | 1.00 | 17.59 |
| ATOM | 4442 | O | ALA | B | 762 | 26.567 | 34.077 | 22.637 | 1.00 | 20.01 |
| ATOM | 4443 | N | CYS | B | 763 | 27.186 | 34.705 | 20.570 | 1.00 | 17.78 |
| ATOM | 4444 | CA | CYS | B | 763 | 27.247 | 36.115 | 20.914 | 1.00 | 18.49 |
| ATOM | 4445 | CB | CYS | B | 763 | 28.041 | 36.869 | 19.850 | 1.00 | 20.27 |
| ATOM | 4446 | SG | CYS | B | 763 | 29.790 | 36.353 | 19.790 | 1.00 | 21.15 |
| ATOM | 4447 | C | CYS | B | 763 | 25.841 | 36.704 | 21.071 | 1.00 | 19.17 |
| ATOM | 4448 | O | CYS | B | 763 | 25.596 | 37.521 | 21.957 | 1.00 | 19.53 |
| ATOM | 4449 | N | ASP | B | 764 | 24.921 | 36.265 | 20.219 | 1.00 | 20.39 |
| ATOM | 4450 | CA | ASP | B | 764 | 23.534 | 36.729 | 20.254 | 1.00 | 20.66 |
| ATOM | 4451 | CB | ASP | B | 764 | 22.739 | 36.046 | 19.130 | 1.00 | 19.31 |
| ATOM | 4452 | CG | ASP | B | 764 | 21.474 | 36.802 | 18.767 | 1.00 | 19.31 |
| ATOM | 4453 | OD1 | ASP | B | 764 | 20.707 | 36.320 | 17.903 | 1.00 | 19.87 |
| ATOM | 4454 | OD2 | ASP | B | 764 | 21.265 | 37.889 | 19.341 | 1.00 | 17.76 |
| ATOM | 4455 | C | ASP | B | 764 | 22.862 | 36.429 | 21.598 | 1.00 | 20.80 |
| ATOM | 4456 | O | ASP | B | 764 | 22.021 | 37.191 | 22.074 | 1.00 | 20.53 |
| ATOM | 4457 | N | LEU | B | 765 | 23.235 | 35.319 | 22.220 | 1.00 | 17.19 |
| ATOM | 4458 | CA | LEU | B | 765 | 22.626 | 34.934 | 23.489 | 1.00 | 17.73 |
| ATOM | 4459 | CB | LEU | B | 765 | 22.337 | 33.428 | 23.476 | 1.00 | 20.86 |
| ATOM | 4460 | CG | LEU | B | 765 | 21.556 | 32.898 | 22.270 | 1.00 | 21.66 |
| ATOM | 4461 | CD1 | LEU | B | 765 | 21.264 | 31.422 | 22.444 | 1.00 | 21.03 |
| ATOM | 4462 | CD2 | LEU | B | 765 | 20.257 | 33.679 | 22.125 | 1.00 | 20.47 |
| ATOM | 4463 | C | LEU | B | 765 | 23.497 | 35.276 | 24.697 | 1.00 | 18.18 |
| ATOM | 4464 | O | LEU | B | 765 | 23.205 | 34.833 | 25.813 | 1.00 | 19.59 |
| ATOM | 4465 | N | SER | B | 766 | 24.516 | 36.111 | 24.475 | 1.00 | 19.68 |
| ATOM | 4466 | CA | SER | B | 766 | 25.498 | 36.475 | 25.504 | 1.00 | 20.44 |
| ATOM | 4467 | CB | SER | B | 766 | 26.478 | 37.527 | 24.959 | 1.00 | 23.53 |
| ATOM | 4468 | OG | SER | B | 766 | 25.937 | 38.837 | 24.998 | 1.00 | 25.78 |
| ATOM | 4469 | C | SER | B | 766 | 24.997 | 36.932 | 26.872 | 1.00 | 20.03 |
| ATOM | 4470 | O | SER | B | 766 | 25.695 | 36.770 | 27.871 | 1.00 | 19.65 |
| ATOM | 4471 | N | ALA | B | 767 | 23.806 | 37.513 | 26.929 | 1.00 | 22.57 |
| ATOM | 4472 | CA | ALA | B | 767 | 23.284 | 37.974 | 28.210 | 1.00 | 23.91 |
| ATOM | 4473 | CB | ALA | B | 767 | 21.943 | 38.682 | 28.002 | 1.00 | 17.04 |
| ATOM | 4474 | C | ALA | B | 767 | 23.123 | 36.832 | 29.213 | 1.00 | 23.55 |
| ATOM | 4475 | O | ALA | B | 767 | 23.202 | 37.045 | 30.426 | 1.00 | 24.64 |
| ATOM | 4476 | N | ILE | B | 768 | 22.911 | 35.621 | 28.703 | 1.00 | 23.20 |
| ATOM | 4477 | CA | ILE | B | 768 | 22.718 | 34.448 | 29.546 | 1.00 | 22.31 |
| ATOM | 4478 | CB | ILE | B | 768 | 22.143 | 33.257 | 28.717 | 1.00 | 23.65 |
| ATOM | 4479 | CG2 | ILE | B | 768 | 23.253 | 32.563 | 27.933 | 1.00 | 22.89 |
| ATOM | 4480 | CG1 | ILE | B | 768 | 21.457 | 32.255 | 29.641 | 1.00 | 23.26 |
| ATOM | 4481 | CD1 | ILE | B | 768 | 20.179 | 32.796 | 30.272 | 1.00 | 23.73 |
| ATOM | 4482 | C | ILE | B | 768 | 24.004 | 33.999 | 30.247 | 1.00 | 22.60 |
| ATOM | 4483 | O | ILE | B | 768 | 23.958 | 33.164 | 31.140 | 1.00 | 22.93 |
| ATOM | 4484 | N | THR | B | 769 | 25.138 | 34.571 | 29.849 | 1.00 | 21.92 |
| ATOM | 4485 | CA | THR | B | 769 | 26.438 | 34.224 | 30.435 | 1.00 | 21.95 |
| ATOM | 4486 | CB | THR | B | 769 | 27.525 | 34.077 | 29.356 | 1.00 | 23.33 |
| ATOM | 4487 | OG1 | THR | B | 769 | 27.793 | 35.362 | 28.778 | 1.00 | 24.40 |
| ATOM | 4488 | CG2 | THR | B | 769 | 27.087 | 33.111 | 28.267 | 1.00 | 24.02 |
| ATOM | 4489 | C | THR | B | 769 | 26.962 | 35.287 | 31.399 | 1.00 | 22.67 |
| ATOM | 4490 | O | THR | B | 769 | 27.973 | 35.086 | 32.073 | 1.00 | 22.65 |
| ATOM | 4491 | N | LYS | B | 770 | 26.284 | 36.425 | 31.452 | 1.00 | 23.25 |
| ATOM | 4492 | CA | LYS | B | 770 | 26.720 | 37.530 | 32.296 | 1.00 | 23.60 |
| ATOM | 4493 | CB | LYS | B | 770 | 25.996 | 38.802 | 31.849 | 1.00 | 21.12 |
| ATOM | 4494 | CG | LYS | B | 770 | 26.258 | 39.179 | 30.403 | 1.00 | 20.96 |
| ATOM | 4495 | CD | LYS | B | 770 | 27.656 | 39.759 | 30.206 | 1.00 | 20.82 |
| ATOM | 4496 | CE | LYS | B | 770 | 27.893 | 40.115 | 28.739 | 1.00 | 20.75 |
| ATOM | 4497 | NZ | LYS | B | 770 | 29.176 | 40.840 | 28.511 | 1.00 | 21.62 |
| ATOM | 4498 | C | LYS | B | 770 | 26.525 | 37.339 | 33.802 | 1.00 | 24.02 |
| ATOM | 4499 | O | LYS | B | 770 | 25.758 | 36.476 | 34.239 | 1.00 | 23.96 |
| ATOM | 4500 | N | PRO | B | 771 | 27.238 | 38.140 | 34.618 | 1.00 | 24.80 |
| ATOM | 4501 | CD | PRO | B | 771 | 28.330 | 39.060 | 34.249 | 1.00 | 25.87 |
| ATOM | 4502 | CA | PRO | B | 771 | 27.109 | 38.045 | 36.076 | 1.00 | 25.95 |
| ATOM | 4503 | CB | PRO | B | 771 | 27.976 | 39.198 | 36.569 | 1.00 | 25.81 |
| ATOM | 4504 | CG | PRO | B | 771 | 29.083 | 39.215 | 35.563 | 1.00 | 26.51 |
| ATOM | 4505 | C | PRO | B | 771 | 25.631 | 38.220 | 36.440 | 1.00 | 26.70 |
| ATOM | 4506 | O | PRO | B | 771 | 24.905 | 38.955 | 35.763 | 1.00 | 25.64 |
| ATOM | 4507 | N | TRP | B | 772 | 25.202 | 37.546 | 37.503 | 1.00 | 27.13 |
| ATOM | 4508 | CA | TRP | B | 772 | 23.811 | 37.553 | 37.959 | 1.00 | 27.52 |
| ATOM | 4509 | CB | TRP | B | 772 | 23.744 | 37.021 | 39.394 | 1.00 | 31.16 |
| ATOM | 4510 | CG | TRP | B | 772 | 22.355 | 36.817 | 39.921 | 1.00 | 31.91 |
| ATOM | 4511 | CD2 | TRP | B | 772 | 21.272 | 36.141 | 39.267 | 1.00 | 31.55 |
| ATOM | 4512 | CE2 | TRP | B | 772 | 20.171 | 36.166 | 40.150 | 1.00 | 31.84 |
| ATOM | 4513 | CE3 | TRP | B | 772 | 21.126 | 35.516 | 38.021 | 1.00 | 31.35 |
| ATOM | 4514 | CD1 | TRP | B | 772 | 21.878 | 37.212 | 41.138 | 1.00 | 31.40 |
| ATOM | 4515 | NE1 | TRP | B | 772 | 20.569 | 36.827 | 41.283 | 1.00 | 32.07 |
| ATOM | 4516 | CZ2 | TRP | B | 772 | 18.937 | 35.591 | 39.828 | 1.00 | 32.13 |
| ATOM | 4517 | CZ3 | TRP | B | 772 | 19.897 | 34.944 | 37.701 | 1.00 | 31.80 |
| ATOM | 4518 | CH2 | TRP | B | 772 | 18.819 | 34.987 | 38.602 | 1.00 | 31.93 |
| ATOM | 4519 | C | TRP | B | 772 | 23.009 | 38.853 | 37.865 | 1.00 | 27.06 |
| ATOM | 4520 | O | TRP | B | 772 | 21.913 | 38.864 | 37.305 | 1.00 | 27.10 |
| ATOM | 4521 | N | PRO | B | 773 | 23.533 | 39.964 | 38.409 | 1.00 | 27.24 |
| ATOM | 4522 | CD | PRO | B | 773 | 24.841 | 40.176 | 39.050 | 1.00 | 36.67 |
| ATOM | 4523 | CA | PRO | B | 773 | 22.778 | 41.220 | 38.336 | 1.00 | 26.63 |
| ATOM | 4524 | CB | PRO | B | 773 | 23.649 | 42.192 | 39.133 | 1.00 | 36.87 |
| ATOM | 4525 | CG | PRO | B | 773 | 25.027 | 41.669 | 38.902 | 1.00 | 37.42 |
| ATOM | 4526 | C | PRO | B | 773 | 22.520 | 41.692 | 36.914 | 1.00 | 25.67 |
| ATOM | 4527 | O | PRO | B | 773 | 21.476 | 42.272 | 36.626 | 1.00 | 26.52 |
| ATOM | 4528 | N | ILE | B | 774 | 23.480 | 41.443 | 36.029 | 1.00 | 21.82 |
| ATOM | 4529 | CA | ILE | B | 774 | 23.341 | 41.834 | 34.636 | 1.00 | 21.26 |
| ATOM | 4530 | CB | ILE | B | 774 | 24.707 | 41.788 | 33.915 | 1.00 | 30.27 |
| ATOM | 4531 | CG2 | ILE | B | 774 | 24.523 | 42.062 | 32.429 | 1.00 | 30.62 |
| ATOM | 4532 | CG1 | ILE | B | 774 | 25.649 | 42.821 | 34.543 | 1.00 | 31.27 |
| ATOM | 4533 | CD1 | ILE | B | 774 | 27.075 | 42.765 | 34.037 | 1.00 | 31.12 |
| ATOM | 4534 | C | ILE | B | 774 | 22.342 | 40.909 | 33.947 | 1.00 | 20.74 |
| ATOM | 4535 | O | ILE | B | 774 | 21.452 | 41.375 | 33.239 | 1.00 | 21.33 |
| ATOM | 4536 | N | GLN | B | 775 | 22.482 | 39.604 | 34.166 | 1.00 | 22.90 |
| ATOM | 4537 | CA | GLN | B | 775 | 21.569 | 38.636 | 33.565 | 1.00 | 23.62 |
| ATOM | 4538 | CB | GLN | B | 775 | 21.965 | 37.205 | 33.956 | 1.00 | 24.73 |
| ATOM | 4539 | CG | GLN | B | 775 | 20.801 | 36.214 | 33.995 | 1.00 | 25.27 |
| ATOM | 4540 | CD | GLN | B | 775 | 20.070 | 36.062 | 32.661 | 1.00 | 25.01 |
| ATOM | 4541 | OE1 | GLN | B | 775 | 18.983 | 35.495 | 32.610 | 1.00 | 23.97 |
| ATOM | 4542 | NE2 | GLN | B | 775 | 20.669 | 36.556 | 31.583 | 1.00 | 25.73 |
| ATOM | 4543 | C | GLN | B | 775 | 20.127 | 38.905 | 33.987 | 1.00 | 24.69 |
| ATOM | 4544 | O | GLN | B | 775 | 19.213 | 38.883 | 33.161 | 1.00 | 23.74 |
| ATOM | 4545 | N | GLN | B | 776 | 19.930 | 39.158 | 35.277 | 1.00 | 24.42 |
| ATOM | 4546 | CA | GLN | B | 776 | 18.596 | 39.440 | 35.800 | 1.00 | 25.46 |
| ATOM | 4547 | CB | GLN | B | 776 | 18.647 | 39.710 | 37.303 | 1.00 | 39.22 |
| ATOM | 4548 | CG | GLN | B | 776 | 18.627 | 38.482 | 38.172 | 1.00 | 40.03 |
| ATOM | 4549 | CD | GLN | B | 776 | 18.641 | 38.828 | 39.648 | 1.00 | 40.44 |
| ATOM | 4550 | OE1 | GLN | B | 776 | 19.635 | 39.339 | 40.167 | 1.00 | 40.18 |
| ATOM | 4551 | NE2 | GLN | B | 776 | 17.536 | 38.554 | 40.332 | 1.00 | 40.01 |
| ATOM | 4552 | C | GLN | B | 776 | 17.989 | 40.658 | 35.121 | 1.00 | 24.70 |
| ATOM | 4553 | O | GLN | B | 776 | 16.832 | 40.641 | 34.712 | 1.00 | 25.09 |
| ATOM | 4554 | N | ARG | B | 777 | 18.773 | 41.725 | 35.028 | 1.00 | 23.31 |
| ATOM | 4555 | CA | ARG | B | 777 | 18.298 | 42.949 | 34.410 | 1.00 | 23.89 |
| ATOM | 4556 | CB | ARG | B | 777 | 19.369 | 44.038 | 34.481 | 1.00 | 27.39 |
| ATOM | 4557 | CG | ARG | B | 777 | 18.902 | 45.369 | 33.917 | 1.00 | 27.72 |
| ATOM | 4558 | CD | ARG | B | 777 | 19.965 | 46.434 | 34.058 | 1.00 | 28.34 |
| ATOM | 4559 | NE | ARG | B | 777 | 21.102 | 46.209 | 33.170 | 1.00 | 27.57 |
| ATOM | 4560 | CZ | ARG | B | 777 | 21.029 | 46.232 | 31.843 | 1.00 | 27.93 |
| ATOM | 4561 | NH1 | ARG | B | 777 | 19.870 | 46.468 | 31.244 | 1.00 | 28.38 |
| ATOM | 4562 | NH2 | ARG | B | 777 | 22.119 | 46.032 | 31.113 | 1.00 | 27.27 |
| ATOM | 4563 | C | ARG | B | 777 | 17.905 | 42.728 | 32.958 | 1.00 | 23.38 |
| ATOM | 4564 | O | ARG | B | 777 | 16.814 | 43.119 | 32.535 | 1.00 | 24.57 |
| ATOM | 4565 | N | ILE | B | 778 | 18.784 | 42.089 | 32.191 | 1.00 | 18.64 |
| ATOM | 4566 | CA | ILE | B | 778 | 18.487 | 41.853 | 30.783 | 1.00 | 19.91 |
| ATOM | 4567 | CB | ILE | B | 778 | 19.729 | 41.350 | 30.028 | 1.00 | 21.48 |
| ATOM | 4568 | CG2 | ILE | B | 778 | 19.371 | 41.065 | 28.575 | 1.00 | 21.88 |
| ATOM | 4569 | CG1 | ILE | B | 778 | 20.809 | 42.432 | 30.077 | 1.00 | 22.67 |
| ATOM | 4570 | CD1 | ILE | B | 778 | 22.127 | 42.024 | 29.472 | 1.00 | 22.44 |
| ATOM | 4571 | C | ILE | B | 778 | 17.333 | 40.880 | 30.605 | 1.00 | 19.30 |
| ATOM | 4572 | O | ILE | B | 778 | 16.494 | 41.063 | 29.718 | 1.00 | 18.16 |
| ATOM | 4573 | N | ALA | B | 779 | 17.272 | 39.865 | 31.459 | 1.00 | 20.27 |
| ATOM | 4574 | CA | ALA | B | 779 | 16.193 | 38.890 | 31.393 | 1.00 | 21.96 |
| ATOM | 4575 | CB | ALA | B | 779 | 16.333 | 37.880 | 32.513 | 1.00 | 23.30 |
| ATOM | 4576 | C | ALA | B | 779 | 14.851 | 39.610 | 31.495 | 1.00 | 21.94 |
| ATOM | 4577 | O | ALA | B | 779 | 13.896 | 39.271 | 30.790 | 1.00 | 21.55 |
| ATOM | 4578 | N | GLU | B | 780 | 14.780 | 40.615 | 32.361 | 1.00 | 23.66 |
| ATOM | 4579 | CA | GLU | B | 780 | 13.539 | 41.368 | 32.520 | 1.00 | 24.79 |
| ATOM | 4580 | CB | GLU | B | 780 | 13.643 | 42.309 | 33.723 | 1.00 | 46.99 |
| ATOM | 4581 | CG | GLU | B | 780 | 13.971 | 41.572 | 35.016 | 1.00 | 47.98 |
| ATOM | 4582 | CD | GLU | B | 780 | 13.473 | 42.287 | 36.255 | 1.00 | 48.28 |
| ATOM | 4583 | OE1 | GLU | B | 780 | 13.748 | 43.499 | 36.398 | 1.00 | 48.22 |
| ATOM | 4584 | OE2 | GLU | B | 780 | 12.814 | 41.630 | 37.089 | 1.00 | 48.29 |
| ATOM | 4585 | C | GLU | B | 780 | 13.171 | 42.144 | 31.251 | 1.00 | 23.73 |
| ATOM | 4586 | O | GLU | B | 780 | 11.989 | 42.308 | 30.931 | 1.00 | 23.36 |
| ATOM | 4587 | N | LEU | B | 781 | 14.180 | 42.615 | 30.525 | 1.00 | 21.19 |
| ATOM | 4588 | CA | LEU | B | 781 | 13.949 | 43.336 | 29.282 | 1.00 | 19.63 |
| ATOM | 4589 | CB | LEU | B | 781 | 15.257 | 43.926 | 28.761 | 1.00 | 24.73 |
| ATOM | 4590 | CG | LEU | B | 781 | 15.902 | 44.956 | 29.687 | 1.00 | 25.34 |
| ATOM | 4591 | CD1 | LEU | B | 781 | 17.189 | 45.469 | 29.065 | 1.00 | 25.01 |
| ATOM | 4592 | CD2 | LEU | B | 781 | 14.926 | 46.101 | 29.913 | 1.00 | 24.85 |
| ATOM | 4593 | C | LEU | B | 781 | 13.393 | 42.353 | 28.261 | 1.00 | 21.36 |
| ATOM | 4594 | O | LEU | B | 781 | 12.510 | 42.686 | 27.466 | 1.00 | 20.39 |
| ATOM | 4595 | N | VAL | B | 782 | 13.918 | 41.134 | 28.286 | 1.00 | 21.98 |
| ATOM | 4596 | CA | VAL | B | 782 | 13.455 | 40.112 | 27.360 | 1.00 | 22.60 |
| ATOM | 4597 | CB | VAL | B | 782 | 14.275 | 38.812 | 27.499 | 1.00 | 22.53 |
| ATOM | 4598 | CG1 | VAL | B | 782 | 13.660 | 37.713 | 26.643 | 1.00 | 22.91 |
| ATOM | 4599 | CG2 | VAL | B | 782 | 15.712 | 39.056 | 27.054 | 1.00 | 23.90 |
| ATOM | 4600 | C | VAL | B | 782 | 11.988 | 39.808 | 27.648 | 1.00 | 22.59 |
| ATOM | 4601 | O | VAL | B | 782 | 11.159 | 39.746 | 26.737 | 1.00 | 22.68 |
| ATOM | 4602 | N | ALA | B | 783 | 11.671 | 39.628 | 28.925 | 1.00 | 20.36 |
| ATOM | 4603 | CA | ALA | B | 783 | 10.301 | 39.327 | 29.323 | 1.00 | 20.69 |
| ATOM | 4604 | CB | ALA | B | 783 | 10.232 | 39.082 | 30.821 | 1.00 | 21.72 |
| ATOM | 4605 | C | ALA | B | 783 | 9.376 | 40.474 | 28.932 | 1.00 | 20.76 |
| ATOM | 4606 | O | ALA | B | 783 | 8.283 | 40.249 | 28.426 | 1.00 | 22.62 |
| ATOM | 4607 | N | THR | B | 784 | 9.823 | 41.704 | 29.155 | 1.00 | 22.97 |
| ATOM | 4608 | CA | THR | B | 784 | 9.013 | 42.862 | 28.807 | 1.00 | 23.28 |
| ATOM | 4609 | CB | THR | B | 784 | 9.752 | 44.182 | 29.139 | 1.00 | 23.34 |
| ATOM | 4610 | OG1 | THR | B | 784 | 9.934 | 44.280 | 30.562 | 1.00 | 23.54 |
| ATOM | 4611 | CG2 | THR | B | 784 | 8.956 | 45.387 | 28.645 | 1.00 | 23.16 |
| ATOM | 4612 | C | THR | B | 784 | 8.643 | 42.846 | 27.322 | 1.00 | 23.86 |
| ATOM | 4613 | O | THR | B | 784 | 7.485 | 43.086 | 26.963 | 1.00 | 24.06 |
| ATOM | 4614 | N | GLU | B | 785 | 9.612 | 42.547 | 26.459 | 1.00 | 20.32 |
| ATOM | 4615 | CA | GLU | B | 785 | 9.333 | 42.519 | 25.029 | 1.00 | 19.62 |
| ATOM | 4616 | CB | GLU | B | 785 | 10.633 | 42.371 | 24.221 | 1.00 | 19.82 |
| ATOM | 4617 | CG | GLU | B | 785 | 10.424 | 42.346 | 22.703 | 1.00 | 19.80 |
| ATOM | 4618 | CD | GLU | B | 785 | 11.708 | 42.572 | 21.908 | 1.00 | 22.07 |
| ATOM | 4619 | OE1 | GLU | B | 785 | 11.800 | 42.066 | 20.765 | 1.00 | 20.56 |
| ATOM | 4620 | OE2 | GLU | B | 785 | 12.620 | 43.264 | 22.408 | 1.00 | 19.00 |
| ATOM | 4621 | C | GLU | B | 785 | 8.360 | 41.386 | 24.709 | 1.00 | 20.53 |
| ATOM | 4622 | O | GLU | B | 785 | 7.395 | 41.588 | 23.964 | 1.00 | 20.48 |
| ATOM | 4623 | N | PHE | B | 786 | 8.603 | 40.205 | 25.276 | 1.00 | 19.27 |
| ATOM | 4624 | CA | PHE | B | 786 | 7.725 | 39.053 | 25.054 | 1.00 | 20.35 |
| ATOM | 4625 | CB | PHE | B | 786 | 8.185 | 37.824 | 25.853 | 1.00 | 27.64 |
| ATOM | 4626 | CG | PHE | B | 786 | 9.394 | 37.122 | 25.285 | 1.00 | 28.61 |
| ATOM | 4627 | CD1 | PHE | B | 786 | 9.799 | 37.330 | 23.971 | 1.00 | 28.46 |
| ATOM | 4628 | CD2 | PHE | B | 786 | 10.111 | 36.225 | 26.072 | 1.00 | 28.06 |
| ATOM | 4629 | CE1 | PHE | B | 786 | 10.903 | 36.652 | 23.451 | 1.00 | 28.92 |
| ATOM | 4630 | CE2 | PHE | B | 786 | 11.212 | 35.543 | 25.561 | 1.00 | 28.43 |
| ATOM | 4631 | CZ | PHE | B | 786 | 11.608 | 35.758 | 24.250 | 1.00 | 28.30 |
| ATOM | 4632 | C | PHE | B | 786 | 6.289 | 39.380 | 25.475 | 1.00 | 20.75 |
| ATOM | 4633 | O | PHE | B | 786 | 5.340 | 39.061 | 24.760 | 1.00 | 18.68 |
| ATOM | 4634 | N | PHE | B | 787 | 6.144 | 40.003 | 26.646 | 1.00 | 22.35 |
| ATOM | 4635 | CA | PHE | B | 787 | 4.825 | 40.361 | 27.160 | 1.00 | 21.89 |
| ATOM | 4636 | CB | PHE | B | 787 | 4.917 | 40.801 | 28.632 | 1.00 | 21.52 |
| ATOM | 4637 | CG | PHE | B | 787 | 5.419 | 39.729 | 29.567 | 1.00 | 22.41 |
| ATOM | 4638 | CD1 | PHE | B | 787 | 5.256 | 38.376 | 29.265 | 1.00 | 22.32 |
| ATOM | 4639 | CD2 | PHE | B | 787 | 6.035 | 40.070 | 30.771 | 1.00 | 21.61 |
| ATOM | 4640 | CE1 | PHE | B | 787 | 5.698 | 37.384 | 30.148 | 1.00 | 22.73 |
| ATOM | 4641 | CE2 | PHE | B | 787 | 6.478 | 39.085 | 31.653 | 1.00 | 22.56 |
| ATOM | 4642 | CZ | PHE | B | 787 | 6.309 | 37.742 | 31.341 | 1.00 | 21.88 |
| ATOM | 4643 | C | PHE | B | 787 | 4.154 | 41.453 | 26.317 | 1.00 | 21.60 |
| ATOM | 4644 | O | PHE | B | 787 | 2.924 | 41.484 | 26.195 | 1.00 | 21.58 |
| ATOM | 4645 | N | ASP | B | 788 | 4.950 | 42.338 | 25.721 | 1.00 | 20.66 |
| ATOM | 4646 | CA | ASP | B | 788 | 4.387 | 43.384 | 24.872 | 1.00 | 21.38 |
| ATOM | 4647 | CB | ASP | B | 788 | 5.459 | 44.404 | 24.467 | 1.00 | 29.62 |
| ATOM | 4648 | CG | ASP | B | 788 | 4.892 | 45.559 | 23.646 | 1.00 | 31.03 |
| ATOM | 4649 | OD1 | ASP | B | 788 | 4.010 | 46.284 | 24.159 | 1.00 | 31.00 |
| ATOM | 4650 | OD2 | ASP | B | 788 | 5.327 | 45.750 | 22.486 | 1.00 | 30.30 |
| ATOM | 4651 | C | ASP | B | 788 | 3.802 | 42.712 | 23.630 | 1.00 | 21.98 |
| ATOM | 4652 | O | ASP | B | 788 | 2.754 | 43.116 | 23.138 | 1.00 | 20.90 |
| ATOM | 4653 | N | GLN | B | 789 | 4.480 | 41.686 | 23.117 | 1.00 | 18.78 |
| ATOM | 4654 | CA | GLN | B | 789 | 3.950 | 40.980 | 21.958 | 1.00 | 19.35 |
| ATOM | 4655 | CB | GLN | B | 789 | 4.947 | 39.965 | 21.397 | 1.00 | 18.41 |
| ATOM | 4656 | CG | GLN | B | 789 | 4.283 | 39.057 | 20.363 | 1.00 | 19.65 |
| ATOM | 4657 | CD | GLN | B | 789 | 5.228 | 38.067 | 19.715 | 1.00 | 19.91 |
| ATOM | 4658 | OE1 | GLN | B | 789 | 4.805 | 37.004 | 19.253 | 1.00 | 20.58 |
| ATOM | 4659 | NE2 | GLN | B | 789 | 6.503 | 38.413 | 19.663 | 1.00 | 17.85 |
| ATOM | 4660 | C | GLN | B | 789 | 2.690 | 40.240 | 22.382 | 1.00 | 19.10 |
| ATOM | 4661 | O | GLN | B | 789 | 1.728 | 40.154 | 21.622 | 1.00 | 19.13 |
| ATOM | 4662 | N | GLY | B | 790 | 2.713 | 39.701 | 23.598 | 1.00 | 20.13 |
| ATOM | 4663 | CA | GLY | B | 790 | 1.565 | 38.983 | 24.118 | 1.00 | 21.62 |
| ATOM | 4664 | C | GLY | B | 790 | 0.338 | 39.870 | 24.089 | 1.00 | 21.39 |
| ATOM | 4665 | O | GLY | B | 790 | −0.748 | 39.426 | 23.711 | 1.00 | 20.51 |
| ATOM | 4666 | N | ASP | B | 791 | 0.506 | 41.127 | 24.491 | 1.00 | 21.64 |
| ATOM | 4667 | CA | ASP | B | 791 | −0.612 | 42.066 | 24.488 | 1.00 | 23.10 |
| ATOM | 4668 | CB | ASP | B | 791 | −0.205 | 43.401 | 25.128 | 1.00 | 27.91 |
| ATOM | 4669 | CG | ASP | B | 791 | 0.161 | 43.263 | 26.599 | 1.00 | 28.04 |
| ATOM | 4670 | OD1 | ASP | B | 791 | −0.368 | 42.352 | 27.271 | 1.00 | 28.51 |
| ATOM | 4671 | OD2 | ASP | B | 791 | 0.967 | 44.083 | 27.092 | 1.00 | 29.30 |
| ATOM | 4672 | C | ASP | B | 791 | −1.118 | 42.307 | 23.061 | 1.00 | 22.57 |
| ATOM | 4673 | O | ASP | B | 791 | −2.328 | 42.422 | 22.837 | 1.00 | 22.71 |
| ATOM | 4674 | N | ARG | B | 792 | −0.196 | 42.388 | 22.103 | 1.00 | 22.55 |
| ATOM | 4675 | CA | ARG | B | 792 | −0.557 | 42.600 | 20.701 | 1.00 | 21.86 |
| ATOM | 4676 | CB | ARG | B | 792 | 0.697 | 42.794 | 19.831 | 1.00 | 25.58 |
| ATOM | 4677 | CG | ARG | B | 792 | 1.559 | 44.015 | 20.155 | 1.00 | 26.82 |
| ATOM | 4678 | CD | ARG | B | 792 | 1.268 | 45.192 | 19.225 | 1.00 | 27.03 |
| ATOM | 4679 | NE | ARG | B | 792 | 1.602 | 44.929 | 17.819 | 1.00 | 26.18 |
| ATOM | 4680 | CZ | ARG | B | 792 | 2.768 | 45.220 | 17.247 | 1.00 | 26.00 |
| ATOM | 4681 | NH1 | ARG | B | 792 | 3.746 | 45.785 | 17.950 | 1.00 | 26.11 |
| ATOM | 4682 | NH2 | ARG | B | 792 | 2.943 | 44.982 | 15.954 | 1.00 | 27.12 |
| ATOM | 4683 | C | ARG | B | 792 | −1.338 | 41.393 | 20.179 | 1.00 | 21.59 |
| ATOM | 4684 | O | ARG | B | 792 | −2.251 | 41.541 | 19.366 | 1.00 | 22.04 |
| ATOM | 4685 | N | GLU | B | 793 | −0.981 | 40.199 | 20.640 | 1.00 | 21.27 |
| ATOM | 4686 | CA | GLU | B | 793 | −1.678 | 38.999 | 20.189 | 1.00 | 23.41 |
| ATOM | 4687 | CB | GLU | B | 793 | −0.915 | 37.741 | 20.613 | 1.00 | 32.13 |
| ATOM | 4688 | CG | GLU | B | 793 | 0.523 | 37.716 | 20.126 | 1.00 | 32.18 |
| ATOM | 4689 | CD | GLU | B | 793 | 1.199 | 36.378 | 20.354 | 1.00 | 32.76 |
| ATOM | 4690 | OE1 | GLU | B | 793 | 0.883 | 35.713 | 21.363 | 1.00 | 33.25 |
| ATOM | 4691 | OE2 | GLU | B | 793 | 2.058 | 35.998 | 19.531 | 1.00 | 32.81 |
| ATOM | 4692 | C | GLU | B | 793 | −3.106 | 38.946 | 20.726 | 1.00 | 23.75 |
| ATOM | 4693 | O | GLU | B | 793 | −4.044 | 38.610 | 19.995 | 1.00 | 22.66 |
| ATOM | 4694 | N | ARG | B | 794 | −3.277 | 39.283 | 21.999 | 1.00 | 24.04 |
| ATOM | 4695 | CA | ARG | B | 794 | −4.603 | 39.256 | 22.599 | 1.00 | 24.34 |
| ATOM | 4696 | CB | ARG | B | 794 | −4.564 | 39.780 | 24.038 | 1.00 | 32.14 |
| ATOM | 4697 | CG | ARG | B | 794 | −3.332 | 39.401 | 24.824 | 1.00 | 33.26 |
| ATOM | 4698 | CD | ARG | B | 794 | −3.543 | 39.603 | 26.316 | 1.00 | 33.20 |
| ATOM | 4699 | NE | ARG | B | 794 | −4.342 | 38.518 | 26.866 | 1.00 | 33.66 |
| ATOM | 4700 | CZ | ARG | B | 794 | −3.964 | 37.749 | 27.881 | 1.00 | 33.17 |
| ATOM | 4701 | NH1 | ARG | B | 794 | −2.794 | 37.948 | 28.472 | 1.00 | 33.10 |
| ATOM | 4702 | NH2 | ARG | B | 794 | −4.748 | 36.759 | 28.285 | 1.00 | 33.54 |
| ATOM | 4703 | C | ARG | B | 794 | −5.566 | 40.119 | 21.797 | 1.00 | 25.06 |
| ATOM | 4704 | O | ARG | B | 794 | −6.713 | 39.740 | 21.563 | 1.00 | 24.85 |
| ATOM | 4705 | N | LYS | B | 795 | −5.090 | 41.279 | 21.369 | 1.00 | 26.49 |
| ATOM | 4706 | CA | LYS | B | 795 | −5.928 | 42.205 | 20.627 | 1.00 | 27.67 |
| ATOM | 4707 | CB | LYS | B | 795 | −5.412 | 43.633 | 20.821 | 1.00 | 43.32 |
| ATOM | 4708 | CG | LYS | B | 795 | −5.021 | 43.940 | 22.260 | 1.00 | 43.98 |
| ATOM | 4709 | CD | LYS | B | 795 | −4.846 | 45.433 | 22.504 | 1.00 | 44.35 |
| ATOM | 4710 | CE | LYS | B | 795 | −6.121 | 46.059 | 23.052 | 1.00 | 44.46 |
| ATOM | 4711 | NZ | LYS | B | 795 | −7.293 | 45.855 | 22.159 | 1.00 | 44.56 |
| ATOM | 4712 | C | LYS | B | 795 | −6.032 | 41.883 | 19.140 | 1.00 | 27.05 |
| ATOM | 4713 | O | LYS | B | 795 | −7.125 | 41.624 | 18.636 | 1.00 | 28.54 |
| ATOM | 4714 | N | GLU | B | 796 | −4.894 | 41.885 | 18.451 | 1.00 | 25.18 |
| ATOM | 4715 | CA | GLU | B | 796 | −4.842 | 41.634 | 17.007 | 1.00 | 24.55 |
| ATOM | 4716 | CB | GLU | B | 796 | −3.464 | 42.009 | 16.464 | 1.00 | 28.07 |
| ATOM | 4717 | CG | GLU | B | 796 | −2.991 | 43.391 | 16.889 | 1.00 | 29.00 |
| ATOM | 4718 | CD | GLU | B | 796 | −1.615 | 43.741 | 16.348 | 1.00 | 28.65 |
| ATOM | 4719 | OE1 | GLU | B | 796 | −0.962 | 44.626 | 16.931 | 1.00 | 29.39 |
| ATOM | 4720 | OE2 | GLU | B | 796 | −1.187 | 43.144 | 15.340 | 1.00 | 29.53 |
| ATOM | 4721 | C | GLU | B | 796 | −5.173 | 40.214 | 16.549 | 1.00 | 24.25 |
| ATOM | 4722 | O | GLU | B | 796 | −5.637 | 40.011 | 15.423 | 1.00 | 25.17 |
| ATOM | 4723 | N | LEU | B | 797 | −4.922 | 39.228 | 17.399 | 1.00 | 22.33 |
| ATOM | 4724 | CA | LEU | B | 797 | −5.201 | 37.847 | 17.021 | 1.00 | 21.19 |
| ATOM | 4725 | CB | LEU | B | 797 | −3.923 | 36.999 | 17.111 | 1.00 | 24.95 |
| ATOM | 4726 | CG | LEU | B | 797 | −2.757 | 37.369 | 16.184 | 1.00 | 24.74 |
| ATOM | 4727 | CD1 | LEU | B | 797 | −1.555 | 36.486 | 16.498 | 1.00 | 24.36 |
| ATOM | 4728 | CD2 | LEU | B | 797 | −3.173 | 37.209 | 14.720 | 1.00 | 25.36 |
| ATOM | 4729 | C | LEU | B | 797 | −6.295 | 37.239 | 17.889 | 1.00 | 22.61 |
| ATOM | 4730 | O | LEU | B | 797 | −6.717 | 36.108 | 17.657 | 1.00 | 21.44 |
| ATOM | 4731 | N | ASN | B | 798 | −6.756 | 38.002 | 18.877 | 1.00 | 22.45 |
| ATOM | 4732 | CA | ASN | B | 798 | −7.807 | 37.556 | 19.791 | 1.00 | 23.65 |
| ATOM | 4733 | CB | ASN | B | 798 | −9.165 | 37.569 | 19.089 | 1.00 | 32.89 |
| ATOM | 4734 | CG | ASN | B | 798 | −10.163 | 38.479 | 19.777 | 1.00 | 34.23 |
| ATOM | 4735 | OD1 | ASN | B | 798 | −10.392 | 38.372 | 20.982 | 1.00 | 32.44 |
| ATOM | 4736 | ND2 | ASN | B | 798 | −10.767 | 39.381 | 19.010 | 1.00 | 35.38 |
| ATOM | 4737 | C | ASN | B | 798 | −7.532 | 36.164 | 20.352 | 1.00 | 23.21 |
| ATOM | 4738 | O | ASN | B | 798 | −8.414 | 35.305 | 20.384 | 1.00 | 23.77 |
| ATOM | 4739 | N | ILE | B | 799 | −6.299 | 35.945 | 20.794 | 1.00 | 22.30 |
| ATOM | 4740 | CA | ILE | B | 799 | −5.914 | 34.655 | 21.359 | 1.00 | 24.01 |
| ATOM | 4741 | CB | ILE | B | 799 | −4.993 | 33.868 | 20.403 | 1.00 | 37.22 |
| ATOM | 4742 | CG2 | ILE | B | 799 | −5.661 | 33.695 | 19.047 | 1.00 | 37.98 |
| ATOM | 4743 | CG1 | ILE | B | 799 | −3.664 | 34.613 | 20.244 | 1.00 | 37.41 |
| ATOM | 4744 | CD1 | ILE | B | 799 | −2.556 | 33.780 | 19.633 | 1.00 | 37.91 |
| ATOM | 4745 | C | ILE | B | 799 | −5.146 | 34.867 | 22.658 | 1.00 | 23.86 |
| ATOM | 4746 | O | ILE | B | 799 | −4.542 | 35.922 | 22.867 | 1.00 | 22.69 |
| ATOM | 4747 | N | GLU | B | 800 | −5.178 | 33.870 | 23.533 | 1.00 | 27.63 |
| ATOM | 4748 | CA | GLU | B | 800 | −4.442 | 33.961 | 24.787 | 1.00 | 28.49 |
| ATOM | 4749 | CB | GLU | B | 800 | −4.912 | 32.882 | 25.766 | 1.00 | 54.96 |
| ATOM | 4750 | CG | GLU | B | 800 | −4.159 | 32.874 | 27.091 | 1.00 | 55.87 |
| ATOM | 4751 | CD | GLU | B | 800 | −4.612 | 31.758 | 28.018 | 1.00 | 56.25 |
| ATOM | 4752 | OE1 | GLU | B | 800 | −4.037 | 31.633 | 29.121 | 1.00 | 56.19 |
| ATOM | 4753 | OE2 | GLU | B | 800 | −5.541 | 31.008 | 27.645 | 1.00 | 56.33 |
| ATOM | 4754 | C | GLU | B | 800 | −2.995 | 33.711 | 24.385 | 1.00 | 28.05 |
| ATOM | 4755 | O | GLU | B | 800 | −2.712 | 32.791 | 23.616 | 1.00 | 28.04 |
| ATOM | 4756 | N | PRO | B | 801 | −2.060 | 34.539 | 24.866 | 1.00 | 26.29 |
| ATOM | 4757 | CD | PRO | B | 801 | −2.159 | 35.766 | 25.673 | 1.00 | 32.02 |
| ATOM | 4758 | CA | PRO | B | 801 | −0.675 | 34.280 | 24.465 | 1.00 | 25.59 |
| ATOM | 4759 | CB | PRO | B | 801 | 0.047 | 35.561 | 24.875 | 1.00 | 32.25 |
| ATOM | 4760 | CG | PRO | B | 801 | −0.719 | 35.993 | 26.085 | 1.00 | 32.69 |
| ATOM | 4761 | C | PRO | B | 801 | −0.115 | 33.044 | 25.155 | 1.00 | 25.64 |
| ATOM | 4762 | O | PRO | B | 801 | −0.688 | 32.552 | 26.127 | 1.00 | 26.27 |
| ATOM | 4763 | N | THR | B | 802 | 0.990 | 32.529 | 24.634 | 1.00 | 27.06 |
| ATOM | 4764 | CA | THR | B | 802 | 1.622 | 31.366 | 25.242 | 1.00 | 27.00 |
| ATOM | 4765 | CB | THR | B | 802 | 2.731 | 30.798 | 24.340 | 1.00 | 41.37 |
| ATOM | 4766 | OG1 | TER | B | 802 | 3.289 | 29.626 | 24.947 | 1.00 | 42.49 |
| ATOM | 4767 | CG2 | THR | B | 802 | 3.824 | 31.831 | 24.124 | 1.00 | 41.44 |
| ATOM | 4768 | C | THR | B | 802 | 2.220 | 31.839 | 26.562 | 1.00 | 26.88 |
| ATOM | 4769 | O | THR | B | 802 | 2.390 | 33.041 | 26.776 | 1.00 | 25.80 |
| ATOM | 4770 | N | ASP | B | 803 | 2.526 | 30.902 | 27.449 | 1.00 | 25.95 |
| ATOM | 4771 | CA | ASP | B | 803 | 3.096 | 31.248 | 28.744 | 1.00 | 25.37 |
| ATOM | 4772 | CB | ASP | B | 803 | 3.534 | 29.974 | 29.469 | 1.00 | 34.61 |
| ATOM | 4773 | CG | ASP | B | 803 | 2.374 | 29.260 | 30.141 | 1.00 | 34.77 |
| ATOM | 4774 | OD1 | ASP | B | 803 | 1.263 | 29.252 | 29.568 | 1.00 | 34.79 |
| ATOM | 4775 | OD2 | ASP | B | 803 | 2.581 | 28.699 | 31.238 | 1.00 | 35.35 |
| ATOM | 4776 | C | ASP | B | 803 | 4.260 | 32.240 | 28.673 | 1.00 | 24.64 |
| ATOM | 4777 | O | ASP | B | 803 | 4.283 | 33.226 | 29.409 | 1.00 | 25.72 |
| ATOM | 4778 | N | LEU | B | 804 | 5.217 | 31.999 | 27.785 | 1.00 | 27.10 |
| ATOM | 4779 | CA | LEU | B | 804 | 6.359 | 32.899 | 27.686 | 1.00 | 26.94 |
| ATOM | 4780 | CB | LEU | B | 804 | 7.423 | 32.300 | 26.757 | 1.00 | 52.26 |
| ATOM | 4781 | CG | LEU | B | 804 | 7.015 | 31.757 | 25.390 | 1.00 | 53.10 |
| ATOM | 4782 | CD1 | LEU | B | 804 | 7.196 | 32.830 | 24.331 | 1.00 | 53.31 |
| ATOM | 4783 | CD2 | LEU | B | 804 | 7.882 | 30.551 | 25.052 | 1.00 | 53.28 |
| ATOM | 4784 | C | LEU | B | 804 | 6.012 | 34.329 | 27.268 | 1.00 | 26.18 |
| ATOM | 4785 | O | LEU | B | 804 | 6.806 | 35.240 | 27.474 | 1.00 | 26.67 |
| ATOM | 4786 | N | MET | B | 805 | 4.823 | 34.537 | 26.705 | 1.00 | 24.63 |
| ATOM | 4787 | CA | MET | B | 805 | 4.423 | 35.880 | 26.285 | 1.00 | 24.58 |
| ATOM | 4788 | CB | MET | B | 805 | 4.151 | 35.902 | 24.780 | 1.00 | 30.72 |
| ATOM | 4789 | CG | MET | B | 805 | 5.333 | 35.419 | 23.963 | 1.00 | 31.20 |
| ATOM | 4790 | SD | MET | B | 805 | 5.137 | 35.642 | 22.209 | 1.00 | 31.94 |
| ATOM | 4791 | CE | MET | B | 805 | 6.846 | 35.924 | 21.734 | 1.00 | 31.24 |
| ATOM | 4792 | C | MET | B | 805 | 3.191 | 36.373 | 27.044 | 1.00 | 23.91 |
| ATOM | 4793 | O | MET | B | 805 | 2.552 | 37.352 | 26.647 | 1.00 | 23.70 |
| ATOM | 4794 | N | ASN | B | 806 | 2.884 | 35.699 | 28.147 | 1.00 | 22.71 |
| ATOM | 4795 | CA | ASN | B | 806 | 1.728 | 36.042 | 28.970 | 1.00 | 23.25 |
| ATOM | 4796 | CB | ASN | B | 806 | 0.917 | 34.772 | 29.255 | 1.00 | 28.25 |
| ATOM | 4797 | CG | ASN | B | 806 | −0.352 | 35.045 | 30.054 | 1.00 | 27.90 |
| ATOM | 4798 | OD1 | ASN | B | 806 | −0.642 | 36.181 | 30.420 | 1.00 | 28.09 |
| ATOM | 4799 | ND2 | ASN | B | 806 | −1.114 | 33.991 | 30.325 | 1.00 | 28.50 |
| ATOM | 4800 | C | ASN | B | 806 | 2.177 | 36.683 | 30.281 | 1.00 | 23.54 |
| ATOM | 4801 | O | ASN | B | 806 | 2.651 | 35.992 | 31.178 | 1.00 | 24.88 |
| ATOM | 4802 | N | ARG | B | 807 | 2.023 | 38.003 | 30.386 | 1.00 | 22.01 |
| ATOM | 4803 | CA | ARG | B | 807 | 2.417 | 38.748 | 31.585 | 1.00 | 22.16 |
| ATOM | 4804 | CB | ARG | B | 807 | 2.040 | 40.223 | 31.426 | 1.00 | 30.21 |
| ATOM | 4805 | CG | ARG | B | 807 | 2.729 | 41.167 | 32.399 | 1.00 | 30.83 |
| ATOM | 4806 | CD | ARG | B | 807 | 2.164 | 42.572 | 32.270 | 1.00 | 30.96 |
| ATOM | 4807 | NE | ARG | B | 807 | 2.118 | 43.013 | 30.879 | 1.00 | 31.52 |
| ATOM | 4808 | CZ | ARG | B | 807 | 3.161 | 43.482 | 30.205 | 1.00 | 31.30 |
| ATOM | 4809 | NH1 | ARG | B | 807 | 4.347 | 43.583 | 30.795 | 1.00 | 31.76 |
| ATOM | 4810 | NH2 | ARG | B | 807 | 3.019 | 43.843 | 28.935 | 1.00 | 30.41 |
| ATOM | 4811 | C | ARG | B | 807 | 1.753 | 38.186 | 32.844 | 1.00 | 23.50 |
| ATOM | 4812 | O | ARG | B | 807 | 2.324 | 38.241 | 33.933 | 1.00 | 22.48 |
| ATOM | 4813 | N | GLU | B | 808 | 0.549 | 37.645 | 32.694 | 1.00 | 27.30 |
| ATOM | 4814 | CA | GLU | B | 808 | −0.164 | 37.075 | 33.833 | 1.00 | 27.61 |
| ATOM | 4815 | CB | GLU | B | 808 | −1.585 | 36.680 | 33.421 | 1.00 | 44.18 |
| ATOM | 4816 | CG | GLU | B | 808 | −2.572 | 37.835 | 33.427 | 1.00 | 44.75 |
| ATOM | 4817 | CD | GLU | B | 808 | −3.887 | 37.494 | 32.750 | 1.00 | 45.10 |
| ATOM | 4818 | OE1 | GLU | B | 808 | −4.418 | 36.388 | 32.990 | 1.00 | 44.66 |
| ATOM | 4819 | OE2 | GLU | B | 808 | −4.390 | 38.342 | 31.982 | 1.00 | 44.84 |
| ATOM | 4820 | C | GLU | B | 808 | 0.559 | 35.865 | 34.416 | 1.00 | 28.07 |
| ATOM | 4821 | O | GLU | B | 808 | 0.426 | 35.572 | 35.604 | 1.00 | 27.52 |
| ATOM | 4822 | N | LYS | B | 809 | 1.328 | 35.171 | 33.582 | 1.00 | 30.73 |
| ATOM | 4823 | CA | LYS | B | 809 | 2.063 | 33.984 | 34.015 | 1.00 | 31.34 |
| ATOM | 4824 | CB | LYS | B | 809 | 1.888 | 32.856 | 32.988 | 1.00 | 44.52 |
| ATOM | 4825 | CG | LYS | B | 809 | 0.443 | 32.434 | 32.739 | 1.00 | 44.86 |
| ATOM | 4826 | CD | LYS | B | 809 | −0.095 | 31.554 | 33.853 | 1.00 | 45.24 |
| ATOM | 4827 | CE | LYS | B | 809 | 0.499 | 30.152 | 33.794 | 1.00 | 45.08 |
| ATOM | 4828 | NZ | LYS | B | 809 | 0.023 | 29.386 | 32.607 | 1.00 | 45.32 |
| ATOM | 4829 | C | LYS | B | 809 | 3.549 | 34.280 | 34.187 | 1.00 | 31.41 |
| ATOM | 4830 | O | LYS | B | 809 | 4.389 | 33.396 | 34.014 | 1.00 | 31.64 |
| ATOM | 4831 | N | LYS | B | 810 | 3.874 | 35.522 | 34.534 | 1.00 | 29.88 |
| ATOM | 4832 | CA | LYS | B | 810 | 5.266 | 35.924 | 34.714 | 1.00 | 29.67 |
| ATOM | 4833 | CB | LYS | B | 810 | 5.350 | 37.417 | 35.033 | 1.00 | 35.65 |
| ATOM | 4834 | CG | LYS | B | 810 | 4.772 | 37.787 | 36.384 | 1.00 | 35.72 |
| ATOM | 4835 | CD | LYS | B | 810 | 4.977 | 39.256 | 36.681 | 1.00 | 36.06 |
| ATOM | 4836 | CE | LYS | B | 810 | 4.445 | 39.617 | 38.058 | 1.00 | 36.21 |
| ATOM | 4837 | NZ | LYS | B | 810 | 4.806 | 41.010 | 38.416 | 1.00 | 36.50 |
| ATOM | 4838 | C | LYS | B | 810 | 5.981 | 35.135 | 35.815 | 1.00 | 29.55 |
| ATOM | 4839 | O | LYS | B | 810 | 7.204 | 35.220 | 35.954 | 1.00 | 29.80 |
| ATOM | 4840 | N | ASN | B | 811 | 5.221 | 34.371 | 36.595 | 1.00 | 28.77 |
| ATOM | 4841 | CA | ASN | B | 811 | 5.809 | 33.576 | 37.672 | 1.00 | 28.26 |
| ATOM | 4842 | CB | ASN | B | 811 | 4.716 | 33.113 | 38.643 | 1.00 | 33.98 |
| ATOM | 4843 | CG | ASN | B | 811 | 3.760 | 32.122 | 38.012 | 1.00 | 33.96 |
| ATOM | 4844 | OD1 | ASN | B | 811 | 4.059 | 30.935 | 37.904 | 1.00 | 34.26 |
| ATOM | 4845 | ND2 | ASN | B | 811 | 2.607 | 32.611 | 37.578 | 1.00 | 34.21 |
| ATOM | 4846 | C | ASN | B | 811 | 6.558 | 32.364 | 37.122 | 1.00 | 28.78 |
| ATOM | 4847 | O | ASN | B | 811 | 7.277 | 31.681 | 37.852 | 1.00 | 28.05 |
| ATOM | 4848 | N | LYS | B | 812 | 6.391 | 32.112 | 35.826 | 1.00 | 27.42 |
| ATOM | 4849 | CA | LYS | B | 812 | 7.034 | 30.983 | 35.161 | 1.00 | 27.06 |
| ATOM | 4850 | CB | LYS | B | 812 | 6.175 | 30.518 | 33.980 | 1.00 | 41.02 |
| ATOM | 4851 | CG | LYS | B | 812 | 5.384 | 29.246 | 34.224 | 1.00 | 41.61 |
| ATOM | 4852 | CD | LYS | B | 812 | 4.485 | 29.360 | 35.436 | 1.00 | 41.66 |
| ATOM | 4853 | CE | LYS | B | 812 | 3.735 | 28.062 | 35.681 | 1.00 | 41.87 |
| ATOM | 4854 | NZ | LYS | B | 812 | 4.665 | 26.921 | 35.913 | 1.00 | 41.60 |
| ATOM | 4855 | C | LYS | B | 812 | 8.436 | 31.306 | 34.655 | 1.00 | 25.94 |
| ATOM | 4856 | O | LYS | B | 812 | 9.169 | 30.409 | 34.248 | 1.00 | 27.20 |
| ATOM | 4857 | N | ILE | B | 813 | 8.814 | 32.579 | 34.693 | 1.00 | 24.47 |
| ATOM | 4858 | CA | ILE | B | 813 | 10.126 | 32.993 | 34.203 | 1.00 | 24.87 |
| ATOM | 4859 | CB | ILE | B | 813 | 10.353 | 34.509 | 34.452 | 1.00 | 27.03 |
| ATOM | 4860 | CG2 | ILE | B | 813 | 11.812 | 34.887 | 34.190 | 1.00 | 26.32 |
| ATOM | 4861 | CG1 | ILE | B | 813 | 9.420 | 35.312 | 33.537 | 1.00 | 27.43 |
| ATOM | 4862 | CD1 | ILE | B | 813 | 9.422 | 36.809 | 33.792 | 1.00 | 27.47 |
| ATOM | 4863 | C | ILE | B | 813 | 11.321 | 32.198 | 34.725 | 1.00 | 24.94 |
| ATOM | 4864 | O | ILE | B | 813 | 12.153 | 31.747 | 33.940 | 1.00 | 24.37 |
| ATOM | 4865 | N | PRO | B | 814 | 11.433 | 32.015 | 36.051 | 1.00 | 23.33 |
| ATOM | 4866 | CD | PRO | B | 814 | 10.649 | 32.549 | 37.180 | 1.00 | 25.56 |
| ATOM | 4867 | CA | PRO | B | 814 | 12.588 | 31.251 | 36.536 | 1.00 | 24.96 |
| ATOM | 4868 | CB | PRO | B | 814 | 12.336 | 31.171 | 38.041 | 1.00 | 25.58 |
| ATOM | 4869 | CG | PRO | B | 814 | 11.650 | 32.478 | 38.317 | 1.00 | 25.37 |
| ATOM | 4870 | C | PRO | B | 814 | 12.708 | 29.870 | 35.892 | 1.00 | 24.30 |
| ATOM | 4871 | O | PRO | B | 814 | 13.752 | 29.519 | 35.346 | 1.00 | 26.00 |
| ATOM | 4872 | N | SER | B | 815 | 11.629 | 29.101 | 35.942 | 1.00 | 27.38 |
| ATOM | 4873 | CA | SER | B | 815 | 11.631 | 27.759 | 35.372 | 1.00 | 28.28 |
| ATOM | 4874 | CB | SER | B | 815 | 10.320 | 27.048 | 35.705 | 1.00 | 30.12 |
| ATOM | 4875 | OG | SER | B | 815 | 9.214 | 27.762 | 35.193 | 1.00 | 30.40 |
| ATOM | 4876 | C | SER | B | 815 | 11.852 | 27.767 | 33.860 | 1.00 | 28.08 |
| ATOM | 4877 | O | SER | B | 815 | 12.426 | 26.831 | 33.307 | 1.00 | 28.51 |
| ATOM | 4878 | N | MET | B | 816 | 11.405 | 28.822 | 33.190 | 1.00 | 26.53 |
| ATOM | 4879 | CA | MET | B | 816 | 11.581 | 28.899 | 31.743 | 1.00 | 25.98 |
| ATOM | 4880 | CB | MET | B | 816 | 10.633 | 29.940 | 31.147 | 1.00 | 38.18 |
| ATOM | 4881 | CG | MET | B | 816 | 9.173 | 29.635 | 31.422 | 1.00 | 38.60 |
| ATOM | 4882 | SD | MET | B | 816 | 8.038 | 30.687 | 30.502 | 1.00 | 40.03 |
| ATOM | 4883 | CE | MET | B | 816 | 8.543 | 32.319 | 31.070 | 1.00 | 38.78 |
| ATOM | 4884 | C | MET | B | 816 | 13.021 | 29.224 | 31.384 | 1.00 | 25.44 |
| ATOM | 4885 | O | MET | B | 816 | 13.540 | 28.736 | 30.383 | 1.00 | 25.22 |
| ATOM | 4886 | N | GLN | B | 817 | 13.666 | 30.047 | 32.207 | 1.00 | 24.56 |
| ATOM | 4887 | CA | GLN | B | 817 | 15.057 | 30.413 | 31.984 | 1.00 | 24.03 |
| ATOM | 4888 | CB | GLN | B | 817 | 15.503 | 31.465 | 33.000 | 1.00 | 23.33 |
| ATOM | 4889 | CG | GLN | B | 817 | 15.074 | 32.885 | 32.672 | 1.00 | 23.25 |
| ATOM | 4890 | CD | GLN | B | 817 | 15.603 | 33.349 | 31.331 | 1.00 | 24.23 |
| ATOM | 4891 | OE1 | GLN | B | 817 | 15.073 | 32.980 | 30.285 | 1.00 | 23.44 |
| ATOM | 4892 | NE2 | GLN | B | 817 | 16.666 | 34.146 | 31.354 | 1.00 | 23.50 |
| ATOM | 4893 | C | GLN | B | 817 | 15.923 | 29.166 | 32.122 | 1.00 | 24.21 |
| ATOM | 4894 | O | GLN | B | 817 | 16.862 | 28.961 | 31.350 | 1.00 | 25.39 |
| ATOM | 4895 | N | VAL | B | 818 | 15.609 | 28.342 | 33.116 | 1.00 | 22.52 |
| ATOM | 4896 | CA | VAL | B | 818 | 16.350 | 27.103 | 33.331 | 1.00 | 24.31 |
| ATOM | 4897 | CB | VAL | B | 818 | 15.820 | 26.335 | 34.562 | 1.00 | 26.47 |
| ATOM | 4898 | CG1 | VAL | B | 818 | 16.541 | 24.993 | 34.688 | 1.00 | 26.36 |
| ATOM | 4899 | CG2 | VAL | B | 818 | 16.037 | 27.164 | 35.815 | 1.00 | 26.36 |
| ATOM | 4900 | C | VAL | B | 818 | 16.182 | 26.236 | 32.085 | 1.00 | 23.82 |
| ATOM | 4901 | O | VAL | B | 818 | 17.148 | 25.659 | 31.588 | 1.00 | 23.44 |
| ATOM | 4902 | N | GLY | B | 819 | 14.953 | 26.172 | 31.577 | 1.00 | 26.53 |
| ATOM | 4903 | CA | GLY | B | 819 | 14.669 | 25.386 | 30.385 | 1.00 | 25.97 |
| ATOM | 4904 | C | GLY | B | 819 | 15.410 | 25.903 | 29.164 | 1.00 | 25.98 |
| ATOM | 4905 | O | GLY | B | 819 | 15.928 | 25.128 | 28.365 | 1.00 | 25.51 |
| ATOM | 4906 | N | PHE | B | 820 | 15.455 | 27.223 | 29.021 | 1.00 | 22.89 |
| ATOM | 4907 | CA | PHE | B | 820 | 16.149 | 27.869 | 27.906 | 1.00 | 22.59 |
| ATOM | 4908 | CB | PHE | B | 820 | 15.941 | 29.389 | 27.997 | 1.00 | 24.78 |
| ATOM | 4909 | CG | PHE | B | 820 | 16.821 | 30.195 | 27.078 | 1.00 | 26.25 |
| ATOM | 4910 | CD1 | PHE | B | 820 | 16.716 | 30.071 | 25.696 | 1.00 | 25.90 |
| ATOM | 4911 | CD2 | PHE | B | 820 | 17.742 | 31.104 | 27.601 | 1.00 | 24.43 |
| ATOM | 4912 | CE1 | PHE | B | 820 | 17.513 | 30.841 | 24.845 | 1.00 | 25.64 |
| ATOM | 4913 | CE2 | PHE | B | 820 | 18.545 | 31.877 | 26.762 | 1.00 | 25.89 |
| ATOM | 4914 | CZ | PHE | B | 820 | 18.432 | 31.747 | 25.381 | 1.00 | 25.35 |
| ATOM | 4915 | C | PHE | B | 820 | 17.641 | 27.535 | 27.953 | 1.00 | 22.74 |
| ATOM | 4916 | O | PHE | B | 820 | 18.256 | 27.231 | 26.932 | 1.00 | 21.86 |
| ATOM | 4917 | N | ILE | B | 821 | 18.222 | 27.595 | 29.143 | 1.00 | 24.60 |
| ATOM | 4918 | CA | ILE | B | 821 | 19.640 | 27.303 | 29.295 | 1.00 | 26.01 |
| ATOM | 4919 | CB | ILE | B | 821 | 20.099 | 27.593 | 30.739 | 1.00 | 23.25 |
| ATOM | 4920 | CG2 | ILE | B | 821 | 21.480 | 27.001 | 30.984 | 1.00 | 23.21 |
| ATOM | 4921 | CG1 | ILE | B | 821 | 20.096 | 29.105 | 30.980 | 1.00 | 22.99 |
| ATOM | 4922 | CD1 | ILE | B | 821 | 20.409 | 29.506 | 32.410 | 1.00 | 23.26 |
| ATOM | 4923 | C | ILE | B | 821 | 19.944 | 25.848 | 28.929 | 1.00 | 26.27 |
| ATOM | 4924 | O | ILE | B | 821 | 20.893 | 25.565 | 28.187 | 1.00 | 26.66 |
| ATOM | 4925 | N | ASP | B | 822 | 19.129 | 24.931 | 29.436 | 1.00 | 29.94 |
| ATOM | 4926 | CA | ASP | B | 822 | 19.332 | 23.511 | 29.168 | 1.00 | 30.98 |
| ATOM | 4927 | CB | ASP | B | 822 | 18.391 | 22.657 | 30.023 | 1.00 | 31.31 |
| ATOM | 4928 | CG | ASP | B | 822 | 18.798 | 22.617 | 31.478 | 1.00 | 31.53 |
| ATOM | 4929 | OD1 | ASP | B | 822 | 19.984 | 22.882 | 31.771 | 1.00 | 31.53 |
| ATOM | 4930 | OD2 | ASP | B | 822 | 17.939 | 22.301 | 32.330 | 1.00 | 31.44 |
| ATOM | 4931 | C | ASP | B | 822 | 19.147 | 23.103 | 27.715 | 1.00 | 30.90 |
| ATOM | 4932 | O | ASP | B | 822 | 19.963 | 22.368 | 27.162 | 1.00 | 31.36 |
| ATOM | 4933 | N | ALA | B | 823 | 18.070 | 23.577 | 27.102 | 1.00 | 28.23 |
| ATOM | 4934 | CA | ALA | B | 823 | 17.757 | 23.217 | 25.724 | 1.00 | 27.21 |
| ATOM | 4935 | CB | ALA | B | 823 | 16.268 | 23.438 | 25.470 | 1.00 | 28.57 |
| ATOM | 4936 | C | ALA | B | 823 | 18.563 | 23.923 | 24.642 | 1.00 | 27.07 |
| ATOM | 4937 | O | ALA | B | 823 | 18.861 | 23.330 | 23.607 | 1.00 | 27.74 |
| ATOM | 4938 | N | ILE | B | 824 | 18.926 | 25.178 | 24.878 | 1.00 | 23.52 |
| ATOM | 4939 | CA | ILE | B | 824 | 19.639 | 25.950 | 23.869 | 1.00 | 23.73 |
| ATOM | 4940 | CB | ILE | B | 824 | 18.802 | 27.205 | 23.482 | 1.00 | 34.66 |
| ATOM | 4941 | CG2 | ILE | B | 824 | 19.646 | 28.198 | 22.686 | 1.00 | 35.04 |
| ATOM | 4942 | CG1 | ILE | B | 824 | 17.571 | 26.780 | 22.682 | 1.00 | 35.26 |
| ATOM | 4943 | CD1 | ILE | B | 824 | 17.912 | 26.136 | 21.354 | 1.00 | 35.75 |
| ATOM | 4944 | C | ILE | B | 824 | 21.052 | 26.416 | 24.195 | 1.00 | 23.62 |
| ATOM | 4945 | O | ILE | B | 824 | 21.963 | 26.256 | 23.387 | 1.00 | 24.76 |
| ATOM | 4946 | N | CYS | B | 825 | 21.242 | 26.982 | 25.381 | 1.00 | 25.35 |
| ATOM | 4947 | CA | CYS | B | 825 | 22.537 | 27.545 | 25.750 | 1.00 | 25.12 |
| ATOM | 4948 | CB | CYS | B | 825 | 22.322 | 28.602 | 26.827 | 1.00 | 22.81 |
| ATOM | 4949 | SG | CYS | B | 825 | 21.059 | 29.783 | 26.356 | 1.00 | 22.83 |
| ATOM | 4950 | C | CYS | B | 825 | 23.700 | 26.653 | 26.169 | 1.00 | 25.45 |
| ATOM | 4951 | O | CYS | B | 825 | 24.796 | 26.778 | 25.627 | 1.00 | 26.37 |
| ATOM | 4952 | N | LEU | B | 826 | 23.477 | 25.783 | 27.145 | 1.00 | 25.06 |
| ATOM | 4953 | CA | LEU | B | 826 | 24.541 | 24.921 | 27.651 | 1.00 | 26.86 |
| ATOM | 4954 | CB | LEU | B | 826 | 23.954 | 23.898 | 28.626 | 1.00 | 41.13 |
| ATOM | 4955 | CG | LEU | B | 826 | 24.523 | 23.958 | 30.048 | 1.00 | 41.95 |
| ATOM | 4956 | CD1 | LEU | B | 826 | 24.537 | 25.392 | 30.550 | 1.00 | 41.82 |
| ATOM | 4957 | CD2 | LEU | B | 826 | 23.695 | 23.076 | 30.967 | 1.00 | 41.66 |
| ATOM | 4958 | C | LEU | B | 826 | 25.397 | 24.208 | 26.606 | 1.00 | 25.29 |
| ATOM | 4959 | O | LEU | B | 826 | 26.623 | 24.327 | 26.619 | 1.00 | 27.23 |
| ATOM | 4960 | N | GLN | B | 827 | 24.764 | 23.467 | 25.703 | 1.00 | 27.86 |
| ATOM | 4961 | CA | GLN | B | 827 | 25.512 | 22.737 | 24.679 | 1.00 | 27.22 |
| ATOM | 4962 | CB | GLN | B | 827 | 24.560 | 21.935 | 23.796 | 1.00 | 43.35 |
| ATOM | 4963 | CG | GLN | B | 827 | 23.663 | 20.981 | 24.546 | 1.00 | 43.82 |
| ATOM | 4964 | CD | GLN | B | 827 | 22.683 | 20.289 | 23.626 | 1.00 | 44.18 |
| ATOM | 4965 | OE1 | GLN | B | 827 | 23.077 | 19.509 | 22.759 | 1.00 | 44.12 |
| ATOM | 4966 | NE2 | GLN | B | 827 | 21.396 | 20.582 | 23.800 | 1.00 | 44.14 |
| ATOM | 4967 | C | GLN | B | 827 | 26.346 | 23.652 | 23.791 | 1.00 | 27.96 |
| ATOM | 4968 | O | GLN | B | 827 | 27.469 | 23.310 | 23.417 | 1.00 | 27.98 |
| ATOM | 4969 | N | LEU | B | 828 | 25.789 | 24.807 | 23.439 | 1.00 | 23.58 |
| ATOM | 4970 | CA | LEU | B | 828 | 26.491 | 25.753 | 22.585 | 1.00 | 23.82 |
| ATOM | 4971 | CB | LEU | B | 828 | 25.574 | 26.922 | 22.212 | 1.00 | 24.08 |
| ATOM | 4972 | CG | LEU | B | 828 | 26.222 | 28.012 | 21.353 | 1.00 | 24.69 |
| ATOM | 4973 | CD1 | LEU | B | 828 | 26.835 | 27.406 | 20.087 | 1.00 | 24.52 |
| ATOM | 4974 | CD2 | LEU | B | 828 | 25.180 | 29.058 | 21.001 | 1.00 | 24.55 |
| ATOM | 4975 | C | LEU | B | 828 | 27.768 | 26.289 | 23.218 | 1.00 | 23.20 |
| ATOM | 4976 | O | LEU | B | 828 | 28.824 | 26.307 | 22.586 | 1.00 | 23.94 |
| ATOM | 4977 | N | TYR | B | 829 | 27.681 | 26.736 | 24.464 | 1.00 | 24.32 |
| ATOM | 4978 | CA | TYR | B | 829 | 28.866 | 27.266 | 25.122 | 1.00 | 24.21 |
| ATOM | 4979 | CB | TYR | B | 829 | 28.461 | 28.040 | 26.378 | 1.00 | 23.28 |
| ATOM | 4980 | CG | TYR | B | 829 | 27.724 | 29.315 | 26.015 | 1.00 | 21.35 |
| ATOM | 4981 | CD1 | TYR | B | 829 | 28.358 | 30.318 | 25.280 | 1.00 | 22.17 |
| ATOM | 4982 | CE1 | TYR | B | 829 | 27.673 | 31.462 | 24.866 | 1.00 | 21.77 |
| ATOM | 4983 | CD2 | TYR | B | 829 | 26.383 | 29.488 | 26.337 | 1.00 | 23.05 |
| ATOM | 4984 | CE2 | TYR | B | 829 | 25.686 | 30.630 | 25.928 | 1.00 | 21.86 |
| ATOM | 4985 | CZ | TYR | B | 829 | 26.338 | 31.609 | 25.192 | 1.00 | 22.76 |
| ATOM | 4986 | OH | TYR | B | 829 | 25.662 | 32.733 | 24.759 | 1.00 | 20.79 |
| ATOM | 4987 | C | TYR | B | 829 | 29.871 | 26.163 | 25.428 | 1.00 | 25.08 |
| ATOM | 4988 | O | TYR | B | 829 | 31.076 | 26.408 | 25.465 | 1.00 | 24.92 |
| ATOM | 4989 | N | GLU | B | 830 | 29.380 | 24.944 | 25.628 | 1.00 | 25.46 |
| ATOM | 4990 | CA | GLU | B | 830 | 30.278 | 23.826 | 25.892 | 1.00 | 27.34 |
| ATOM | 4991 | CB | GLU | B | 830 | 29.482 | 22.572 | 26.268 | 1.00 | 35.78 |
| ATOM | 4992 | CG | GLU | B | 830 | 28.822 | 22.637 | 27.635 | 1.00 | 36.70 |
| ATOM | 4993 | CD | GLU | B | 830 | 27.981 | 21.409 | 27.938 | 1.00 | 36.51 |
| ATOM | 4994 | OE1 | GLU | B | 830 | 27.433 | 21.318 | 29.056 | 1.00 | 36.91 |
| ATOM | 4995 | OE2 | GLU | B | 830 | 27.863 | 20.533 | 27.053 | 1.00 | 37.38 |
| ATOM | 4996 | C | GLU | B | 830 | 31.087 | 23.563 | 24.624 | 1.00 | 26.06 |
| ATOM | 4997 | O | GLU | B | 830 | 32.305 | 23.377 | 24.679 | 1.00 | 26.54 |
| ATOM | 4998 | N | ALA | B | 831 | 30.398 | 23.566 | 23.486 | 1.00 | 25.64 |
| ATOM | 4999 | CA | ALA | B | 831 | 31.026 | 23.332 | 22.189 | 1.00 | 23.78 |
| ATOM | 5000 | CB | ALA | B | 831 | 29.952 | 23.253 | 21.093 | 1.00 | 30.54 |
| ATOM | 5001 | C | ALA | B | 831 | 32.031 | 24.427 | 21.853 | 1.00 | 24.89 |
| ATOM | 5002 | O | ALA | B | 831 | 33.110 | 24.151 | 21.328 | 1.00 | 24.70 |
| ATOM | 5003 | N | LEU | B | 832 | 31.679 | 25.671 | 22.158 | 1.00 | 22.52 |
| ATOM | 5004 | CA | LEU | B | 832 | 32.578 | 26.778 | 21.872 | 1.00 | 23.49 |
| ATOM | 5005 | CB | LEU | B | 832 | 31.905 | 28.116 | 22.198 | 1.00 | 21.26 |
| ATOM | 5006 | CG | LEU | B | 832 | 32.754 | 29.333 | 21.824 | 1.00 | 22.68 |
| ATOM | 5007 | CD1 | LEU | B | 832 | 33.199 | 29.213 | 20.369 | 1.00 | 21.78 |
| ATOM | 5008 | CD2 | LEU | B | 832 | 31.963 | 30.613 | 22.042 | 1.00 | 22.23 |
| ATOM | 5009 | C | LEU | B | 832 | 33.854 | 26.627 | 22.694 | 1.00 | 22.63 |
| ATOM | 5010 | O | LEU | B | 832 | 34.955 | 26.916 | 22.220 | 1.00 | 23.62 |
| ATOM | 5011 | N | THR | B | 833 | 33.697 | 26.165 | 23.928 | 1.00 | 26.15 |
| ATOM | 5012 | CA | THR | B | 833 | 34.828 | 25.973 | 24.822 | 1.00 | 27.69 |
| ATOM | 5013 | CB | THR | B | 833 | 34.350 | 25.621 | 26.247 | 1.00 | 25.51 |
| ATOM | 5014 | OG1 | THR | B | 833 | 33.549 | 26.695 | 26.759 | 1.00 | 27.15 |
| ATOM | 5015 | CG2 | THR | B | 833 | 35.539 | 25.401 | 27.181 | 1.00 | 26.42 |
| ATOM | 5016 | C | THR | B | 833 | 35.733 | 24.862 | 24.293 | 1.00 | 27.86 |
| ATOM | 5017 | O | THR | B | 833 | 36.949 | 24.876 | 24.506 | 1.00 | 27.78 |
| ATOM | 5018 | N | HIS | B | 834 | 35.132 | 23.904 | 23.600 | 1.00 | 29.42 |
| ATOM | 5019 | CA | HIS | B | 834 | 35.885 | 22.795 | 23.026 | 1.00 | 30.11 |
| ATOM | 5020 | CB | HIS | B | 834 | 34.920 | 21.755 | 22.449 | 1.00 | 46.85 |
| ATOM | 5021 | CG | HIS | B | 834 | 35.571 | 20.457 | 22.084 | 1.00 | 47.63 |
| ATOM | 5022 | CD2 | HIS | B | 834 | 36.207 | 20.064 | 20.955 | 1.00 | 47.79 |
| ATOM | 5023 | ND1 | HIS | B | 834 | 35.621 | 19.382 | 22.945 | 1.00 | 47.75 |
| ATOM | 5024 | CE1 | HIS | B | 834 | 36.257 | 18.381 | 22.361 | 1.00 | 47.72 |
| ATOM | 5025 | NE2 | HIS | B | 834 | 36.623 | 18.769 | 21.153 | 1.00 | 47.50 |
| ATOM | 5026 | C | HIS | B | 834 | 36.773 | 23.351 | 21.912 | 1.00 | 30.08 |
| ATOM | 5027 | O | HIS | B | 834 | 37.939 | 22.972 | 21.773 | 1.00 | 30.93 |
| ATOM | 5028 | N | VAL | B | 835 | 36.209 | 24.259 | 21.119 | 1.00 | 25.19 |
| ATOM | 5029 | CA | VAL | B | 835 | 36.935 | 24.866 | 20.015 | 1.00 | 25.37 |
| ATOM | 5030 | CB | VAL | B | 835 | 35.986 | 25.730 | 19.144 | 1.00 | 27.06 |
| ATOM | 5031 | CG1 | VAL | B | 835 | 36.764 | 26.460 | 18.071 | 1.00 | 27.88 |
| ATOM | 5032 | CG2 | VAL | B | 835 | 34.932 | 24.842 | 18.505 | 1.00 | 27.32 |
| ATOM | 5033 | C | VAL | B | 835 | 38.107 | 25.704 | 20.515 | 1.00 | 25.36 |
| ATOM | 5034 | O | VAL | B | 835 | 39.204 | 25.626 | 19.971 | 1.00 | 25.68 |
| ATOM | 5035 | N | SER | B | 836 | 37.874 | 26.504 | 21.552 | 1.00 | 28.63 |
| ATOM | 5036 | CA | SER | B | 836 | 38.926 | 27.340 | 22.130 | 1.00 | 29.30 |
| ATOM | 5037 | CB | SER | B | 836 | 38.924 | 28.734 | 21.489 | 1.00 | 32.06 |
| ATOM | 5038 | OG | SER | B | 836 | 39.982 | 29.529 | 21.996 | 1.00 | 32.73 |
| ATOM | 5039 | C | SER | B | 836 | 38.674 | 27.454 | 23.629 | 1.00 | 29.51 |
| ATOM | 5040 | O | SER | B | 836 | 37.718 | 28.096 | 24.058 | 1.00 | 28.84 |
| ATOM | 5041 | N | GLU | B | 837 | 39.530 | 26.825 | 24.426 | 1.00 | 26.81 |
| ATOM | 5042 | CA | GLU | B | 837 | 39.359 | 26.846 | 25.874 | 1.00 | 27.10 |
| ATOM | 5043 | CB | GLU | B | 837 | 40.430 | 25.972 | 26.536 | 1.00 | 50.60 |
| ATOM | 5044 | CG | GLU | B | 837 | 41.843 | 26.245 | 26.062 | 1.00 | 51.48 |
| ATOM | 5045 | CD | GLU | B | 837 | 42.811 | 25.145 | 26.460 | 1.00 | 51.89 |
| ATOM | 5046 | OE1 | GLU | B | 837 | 42.985 | 24.912 | 27.677 | 1.00 | 51.52 |
| ATOM | 5047 | OE2 | GLU | B | 837 | 43.394 | 24.512 | 25.552 | 1.00 | 51.73 |
| ATOM | 5048 | C | GLU | B | 837 | 39.348 | 28.242 | 26.494 | 1.00 | 26.43 |
| ATOM | 5049 | O | GLU | B | 837 | 38.902 | 28.413 | 27.628 | 1.00 | 27.28 |
| ATOM | 5050 | N | ASP | B | 838 | 39.819 | 29.238 | 25.749 | 1.00 | 24.88 |
| ATOM | 5051 | CA | ASP | B | 838 | 39.846 | 30.604 | 26.254 | 1.00 | 25.77 |
| ATOM | 5052 | CB | ASP | B | 838 | 40.811 | 31.457 | 25.428 | 1.00 | 38.63 |
| ATOM | 5053 | CG | ASP | B | 838 | 42.256 | 31.011 | 25.582 | 1.00 | 39.53 |
| ATOM | 5054 | OD1 | ASP | B | 838 | 42.765 | 31.036 | 26.724 | 1.00 | 39.53 |
| ATOM | 5055 | OD2 | ASP | B | 838 | 42.877 | 30.633 | 24.568 | 1.00 | 39.29 |
| ATOM | 5056 | C | ASP | B | 838 | 38.449 | 31.226 | 26.252 | 1.00 | 25.03 |
| ATOM | 5057 | O | ASP | B | 838 | 38.277 | 32.360 | 26.699 | 1.00 | 25.25 |
| ATOM | 5058 | N | CYS | B | 839 | 37.464 | 30.483 | 25.748 | 1.00 | 29.32 |
| ATOM | 5059 | CA | CYS | B | 839 | 36.073 | 30.949 | 25.721 | 1.00 | 28.67 |
| ATOM | 5060 | CB | CYS | B | 839 | 35.383 | 30.561 | 24.400 | 1.00 | 25.46 |
| ATOM | 5061 | SG | CYS | B | 839 | 35.809 | 31.580 | 22.967 | 1.00 | 23.35 |
| ATOM | 5062 | C | CYS | B | 839 | 35.299 | 30.342 | 26.895 | 1.00 | 29.69 |
| ATOM | 5063 | O | CYS | B | 839 | 34.075 | 30.468 | 26.984 | 1.00 | 29.52 |
| ATOM | 5064 | N | PHE | B | 840 | 36.023 | 29.686 | 27.798 | 1.00 | 26.11 |
| ATOM | 5065 | CA | PHE | B | 840 | 35.405 | 29.055 | 28.960 | 1.00 | 25.54 |
| ATOM | 5066 | CB | PHE | B | 840 | 36.467 | 28.387 | 29.834 | 1.00 | 29.48 |
| ATOM | 5067 | CG | PHE | B | 840 | 35.916 | 27.797 | 31.101 | 1.00 | 29.46 |
| ATOM | 5068 | CD1 | PHE | B | 840 | 35.084 | 26.679 | 31.060 | 1.00 | 29.72 |
| ATOM | 5069 | CD2 | PHE | B | 840 | 36.204 | 28.373 | 32.333 | 1.00 | 29.95 |
| ATOM | 5070 | CE1 | PHE | B | 840 | 34.546 | 26.143 | 32.231 | 1.00 | 30.22 |
| ATOM | 5071 | CE2 | PHE | B | 840 | 35.671 | 27.845 | 33.512 | 1.00 | 29.42 |
| ATOM | 5072 | CZ | PHE | B | 840 | 34.840 | 26.729 | 33.460 | 1.00 | 29.68 |
| ATOM | 5073 | C | PHE | B | 840 | 34.567 | 29.996 | 29.831 | 1.00 | 26.06 |
| ATOM | 5074 | O | PHE | B | 840 | 33.531 | 29.593 | 30.354 | 1.00 | 26.34 |
| ATOM | 5075 | N | PRO | B | 841 | 35.007 | 31.253 | 30.013 | 1.00 | 25.13 |
| ATOM | 5076 | CD | PRO | B | 841 | 36.255 | 31.904 | 29.575 | 1.00 | 24.36 |
| ATOM | 5077 | CA | PRO | B | 841 | 34.210 | 32.164 | 30.846 | 1.00 | 24.99 |
| ATOM | 5078 | CB | PRO | B | 841 | 34.927 | 33.502 | 30.673 | 1.00 | 24.78 |
| ATOM | 5079 | CG | PRO | B | 841 | 36.368 | 33.073 | 30.535 | 1.00 | 24.46 |
| ATOM | 5080 | C | PRO | B | 841 | 32.729 | 32.229 | 30.446 | 1.00 | 24.81 |
| ATOM | 5081 | O | PRO | B | 841 | 31.865 | 32.393 | 31.300 | 1.00 | 25.33 |
| ATOM | 5082 | N | LEU | B | 842 | 32.435 | 32.096 | 29.155 | 1.00 | 24.08 |
| ATOM | 5083 | CA | LEU | B | 842 | 31.044 | 32.143 | 28.707 | 1.00 | 22.49 |
| ATOM | 5084 | CB | LEU | B | 842 | 30.972 | 32.171 | 27.177 | 1.00 | 19.33 |
| ATOM | 5085 | CG | LEU | B | 842 | 31.614 | 33.377 | 26.488 | 1.00 | 20.20 |
| ATOM | 5086 | CD1 | LEU | B | 842 | 31.712 | 33.118 | 24.985 | 1.00 | 19.25 |
| ATOM | 5087 | CD2 | LEU | B | 842 | 30.794 | 34.632 | 26.783 | 1.00 | 20.71 |
| ATOM | 5088 | C | LEU | B | 842 | 30.260 | 30.944 | 29.242 | 1.00 | 22.87 |
| ATOM | 5089 | O | LEU | B | 842 | 29.143 | 31.088 | 29.732 | 1.00 | 23.13 |
| ATOM | 5090 | N | LEU | B | 843 | 30.855 | 29.760 | 29.158 | 1.00 | 22.66 |
| ATOM | 5091 | CA | LEU | B | 843 | 30.195 | 28.553 | 29.640 | 1.00 | 22.53 |
| ATOM | 5092 | CB | LEU | B | 843 | 31.032 | 27.317 | 29.298 | 1.00 | 33.57 |
| ATOM | 5093 | CG | LEU | B | 843 | 30.366 | 25.935 | 29.275 | 1.00 | 34.94 |
| ATOM | 5094 | CD1 | LEU | B | 843 | 31.458 | 24.882 | 29.157 | 1.00 | 34.20 |
| ATOM | 5095 | CD2 | LEU | B | 843 | 29.540 | 25.691 | 30.515 | 1.00 | 34.62 |
| ATOM | 5096 | C | LEU | B | 843 | 30.024 | 28.635 | 31.153 | 1.00 | 22.75 |
| ATOM | 5097 | O | LEU | B | 843 | 28.976 | 28.280 | 31.690 | 1.00 | 22.24 |
| ATOM | 5098 | N | ASP | B | 844 | 31.068 | 29.101 | 31.831 | 1.00 | 25.80 |
| ATOM | 5099 | CA | ASP | B | 844 | 31.061 | 29.228 | 33.283 | 1.00 | 26.51 |
| ATOM | 5100 | CB | ASP | B | 844 | 32.425 | 29.733 | 33.761 | 1.00 | 37.17 |
| ATOM | 5101 | CG | ASP | B | 844 | 32.584 | 29.653 | 35.267 | 1.00 | 37.71 |
| ATOM | 5102 | OD1 | ASP | B | 844 | 32.277 | 28.589 | 35.844 | 1.00 | 37.75 |
| ATOM | 5103 | OD2 | ASP | B | 844 | 33.028 | 30.654 | 35.871 | 1.00 | 38.29 |
| ATOM | 5104 | C | ASP | B | 844 | 29.953 | 30.171 | 33.750 | 1.00 | 26.88 |
| ATOM | 5105 | O | ASP | B | 844 | 29.215 | 29.855 | 34.683 | 1.00 | 25.76 |
| ATOM | 5106 | N | GLY | B | 845 | 29.840 | 31.323 | 33.093 | 1.00 | 27.19 |
| ATOM | 5107 | CA | GLY | B | 845 | 28.811 | 32.281 | 33.457 | 1.00 | 27.20 |
| ATOM | 5108 | C | GLY | B | 845 | 27.418 | 31.719 | 33.239 | 1.00 | 27.51 |
| ATOM | 5109 | O | GLY | B | 845 | 26.511 | 31.944 | 34.043 | 1.00 | 26.76 |
| ATOM | 5110 | N | CYS | B | 846 | 27.246 | 30.982 | 32.147 | 1.00 | 23.86 |
| ATOM | 5111 | CA | CYS | B | 846 | 25.956 | 30.381 | 31.838 | 1.00 | 24.52 |
| ATOM | 5112 | CB | CYS | B | 846 | 26.026 | 29.636 | 30.505 | 1.00 | 23.78 |
| ATOM | 5113 | SG | CYS | B | 846 | 24.460 | 28.924 | 29.962 | 1.00 | 22.31 |
| ATOM | 5114 | C | CYS | B | 846 | 25.538 | 29.414 | 32.938 | 1.00 | 24.60 |
| ATOM | 5115 | O | CYS | B | 846 | 24.388 | 29.414 | 33.373 | 1.00 | 24.31 |
| ATOM | 5116 | N | ARG | B | 847 | 26.477 | 28.589 | 33.394 | 1.00 | 26.53 |
| ATOM | 5117 | CA | ARG | B | 847 | 26.174 | 27.621 | 34.438 | 1.00 | 26.50 |
| ATOM | 5118 | CB | ARG | B | 847 | 27.317 | 26.605 | 34.556 | 1.00 | 46.23 |
| ATOM | 5119 | CG | ARG | B | 847 | 27.486 | 25.797 | 33.273 | 1.00 | 46.99 |
| ATOM | 5120 | CD | ARG | B | 847 | 28.277 | 24.507 | 33.443 | 1.00 | 47.45 |
| ATOM | 5121 | NE | ARG | B | 847 | 29.724 | 24.700 | 33.406 | 1.00 | 47.23 |
| ATOM | 5122 | CZ | ARG | B | 847 | 30.594 | 23.737 | 33.111 | 1.00 | 47.33 |
| ATOM | 5123 | NH1 | ARG | B | 847 | 30.165 | 22.517 | 32.822 | 1.00 | 47.47 |
| ATOM | 5124 | NH2 | ARG | B | 847 | 31.896 | 23.989 | 33.109 | 1.00 | 46.97 |
| ATOM | 5125 | C | ARG | B | 847 | 25.893 | 28.301 | 35.776 | 1.00 | 26.91 |
| ATOM | 5126 | O | ARG | B | 847 | 25.046 | 27.842 | 36.549 | 1.00 | 27.77 |
| ATOM | 5127 | N | LYS | B | 848 | 26.592 | 29.400 | 36.039 | 1.00 | 28.28 |
| ATOM | 5128 | CA | LYS | B | 848 | 26.384 | 30.155 | 37.271 | 1.00 | 27.83 |
| ATOM | 5129 | CB | LYS | B | 848 | 27.420 | 31.277 | 37.399 | 1.00 | 32.07 |
| ATOM | 5130 | CG | LYS | B | 848 | 28.831 | 30.798 | 37.731 | 1.00 | 32.23 |
| ATOM | 5131 | CD | LYS | B | 848 | 29.798 | 31.975 | 37.858 | 1.00 | 32.55 |
| ATOM | 5132 | CE | LYS | B | 848 | 31.198 | 31.513 | 38.248 | 1.00 | 32.52 |
| ATOM | 5133 | NZ | LYS | B | 848 | 32.179 | 32.642 | 38.241 | 1.00 | 33.18 |
| ATOM | 5134 | C | LYS | B | 848 | 24.973 | 30.740 | 37.257 | 1.00 | 28.34 |
| ATOM | 5135 | O | LYS | B | 848 | 24.291 | 30.758 | 38.282 | 1.00 | 28.54 |
| ATOM | 5136 | N | ASN | B | 849 | 24.525 | 31.213 | 36.097 | 1.00 | 24.72 |
| ATOM | 5137 | CA | ASN | B | 849 | 23.179 | 31.765 | 36.026 | 1.00 | 24.90 |
| ATOM | 5138 | CB | ASN | B | 849 | 22.975 | 32.558 | 34.734 | 1.00 | 24.86 |
| ATOM | 5139 | CG | ASN | B | 849 | 23.561 | 33.949 | 34.817 | 1.00 | 25.77 |
| ATOM | 5140 | OD1 | ASN | B | 849 | 23.482 | 34.602 | 35.865 | 1.00 | 26.99 |
| ATOM | 5141 | ND2 | ASN | B | 849 | 24.139 | 34.422 | 33.717 | 1.00 | 25.44 |
| ATOM | 5142 | C | ASN | B | 849 | 22.117 | 30.676 | 36.149 | 1.00 | 24.90 |
| ATOM | 5143 | O | ASN | B | 849 | 21.041 | 30.909 | 36.698 | 1.00 | 25.65 |
| ATOM | 5144 | N | ARG | B | 850 | 22.410 | 29.484 | 35.639 | 1.00 | 26.25 |
| ATOM | 5145 | CA | ARG | B | 850 | 21.452 | 28.392 | 35.738 | 1.00 | 26.05 |
| ATOM | 5146 | CB | ARG | B | 850 | 21.991 | 27.128 | 35.071 | 1.00 | 37.42 |
| ATOM | 5147 | CG | ARG | B | 850 | 21.057 | 25.945 | 35.237 | 1.00 | 37.84 |
| ATOM | 5148 | CD | ARG | B | 850 | 21.721 | 24.635 | 34.879 | 1.00 | 37.92 |
| ATOM | 5149 | NE | ARG | B | 850 | 20.875 | 23.509 | 35.261 | 1.00 | 38.86 |
| ATOM | 5150 | CZ | ARG | B | 850 | 19.666 | 23.286 | 34.761 | 1.00 | 38.08 |
| ATOM | 5151 | NH1 | ARG | B | 850 | 19.168 | 24.114 | 33.854 | 1.00 | 39.16 |
| ATOM | 5152 | NH2 | ARG | B | 850 | 18.950 | 22.247 | 35.172 | 1.00 | 37.88 |
| ATOM | 5153 | C | ARG | B | 850 | 21.209 | 28.112 | 37.217 | 1.00 | 26.02 |
| ATOM | 5154 | O | ARG | B | 850 | 20.087 | 27.861 | 37.646 | 1.00 | 25.93 |
| ATOM | 5155 | N | GLN | B | 851 | 22.290 | 28.148 | 37.986 | 1.00 | 29.35 |
| ATOM | 5156 | CA | GLN | B | 851 | 22.231 | 27.915 | 39.419 | 1.00 | 29.80 |
| ATOM | 5157 | CB | GLN | B | 851 | 23.641 | 28.042 | 40.006 | 1.00 | 57.33 |
| ATOM | 5158 | CG | GLN | B | 851 | 23.706 | 28.453 | 41.467 | 1.00 | 58.18 |
| ATOM | 5159 | CD | GLN | B | 851 | 25.130 | 28.731 | 41.920 | 1.00 | 58.63 |
| ATOM | 5160 | OE1 | GLN | B | 851 | 25.355 | 29.314 | 42.983 | 1.00 | 58.50 |
| ATOM | 5161 | NE2 | GLN | B | 851 | 26.101 | 28.309 | 41.114 | 1.00 | 58.38 |
| ATOM | 5162 | C | GLN | B | 851 | 21.289 | 28.920 | 40.068 | 1.00 | 29.02 |
| ATOM | 5163 | O | GLN | B | 851 | 20.413 | 28.550 | 40.852 | 1.00 | 29.23 |
| ATOM | 5164 | N | LYS | B | 852 | 21.464 | 30.192 | 39.724 | 1.00 | 28.14 |
| ATOM | 5165 | CA | LYS | B | 852 | 20.635 | 31.253 | 40.283 | 1.00 | 28.58 |
| ATOM | 5166 | CB | LYS | B | 852 | 21.150 | 32.616 | 39.815 | 1.00 | 42.42 |
| ATOM | 5167 | CG | LYS | B | 852 | 22.495 | 33.007 | 40.409 | 1.00 | 42.77 |
| ATOM | 5168 | CD | LYS | B | 852 | 22.371 | 33.238 | 41.906 | 1.00 | 42.79 |
| ATOM | 5169 | CE | LYS | B | 852 | 23.700 | 33.633 | 42.527 | 1.00 | 43.26 |
| ATOM | 5170 | NZ | LYS | B | 852 | 23.538 | 34.014 | 43.959 | 1.00 | 43.16 |
| ATOM | 5171 | C | LYS | B | 852 | 19.153 | 31.116 | 39.941 | 1.00 | 28.34 |
| ATOM | 5172 | O | LYS | B | 852 | 18.296 | 31.254 | 40.819 | 1.00 | 28.28 |
| ATOM | 5173 | N | TRP | B | 853 | 18.846 | 30.854 | 38.672 | 1.00 | 28.99 |
| ATOM | 5174 | CA | TRP | B | 853 | 17.454 | 30.709 | 38.252 | 1.00 | 29.17 |
| ATOM | 5175 | CB | TRP | B | 853 | 17.355 | 30.629 | 36.719 | 1.00 | 25.14 |
| ATOM | 5176 | CG | TRP | B | 853 | 17.517 | 31.963 | 36.032 | 1.00 | 23.87 |
| ATOM | 5177 | CD2 | TRP | B | 853 | 16.695 | 33.121 | 36.210 | 1.00 | 23.86 |
| ATOM | 5178 | CE2 | TRP | B | 853 | 17.216 | 34.136 | 35.377 | 1.00 | 23.34 |
| ATOM | 5179 | CE3 | TRP | B | 853 | 15.567 | 33.400 | 36.995 | 1.00 | 24.36 |
| ATOM | 5180 | CD1 | TRP | B | 853 | 18.475 | 32.311 | 35.121 | 1.00 | 24.42 |
| ATOM | 5181 | NE1 | TRP | B | 853 | 18.302 | 33.614 | 34.725 | 1.00 | 24.36 |
| ATOM | 5182 | CZ2 | TRP | B | 853 | 16.648 | 35.412 | 35.305 | 1.00 | 23.69 |
| ATOM | 5183 | CZ3 | TRP | B | 853 | 15.002 | 34.669 | 36.924 | 1.00 | 23.64 |
| ATOM | 5184 | CH2 | TRP | B | 853 | 15.545 | 35.657 | 36.085 | 1.00 | 24.37 |
| ATOM | 5185 | C | TRP | B | 853 | 16.783 | 29.488 | 38.882 | 1.00 | 29.77 |
| ATOM | 5186 | O | TRP | B | 853 | 15.632 | 29.561 | 39.309 | 1.00 | 29.78 |
| ATOM | 5187 | N | GLN | B | 854 | 17.504 | 28.371 | 38.946 | 1.00 | 29.55 |
| ATOM | 5188 | CA | GLN | B | 854 | 16.951 | 27.150 | 39.526 | 1.00 | 30.02 |
| ATOM | 5189 | CB | GLN | B | 854 | 17.937 | 25.993 | 39.358 | 1.00 | 34.76 |
| ATOM | 5190 | CG | GLN | B | 854 | 17.451 | 24.662 | 39.918 | 1.00 | 34.86 |
| ATOM | 5191 | CD | GLN | B | 854 | 16.141 | 24.207 | 39.305 | 1.00 | 35.26 |
| ATOM | 5192 | OE1 | GLN | B | 854 | 15.984 | 24.197 | 38.085 | 1.00 | 35.00 |
| ATOM | 5193 | NE2 | GLN | B | 854 | 15.195 | 23.815 | 40.151 | 1.00 | 35.46 |
| ATOM | 5194 | C | GLN | B | 854 | 16.611 | 27.328 | 41.005 | 1.00 | 30.99 |
| ATOM | 5195 | O | GLN | B | 854 | 15.559 | 26.884 | 41.462 | 1.00 | 30.12 |
| ATOM | 5196 | N | ALA | B | 855 | 17.501 | 27.976 | 41.748 | 1.00 | 37.27 |
| ATOM | 5197 | CA | ALA | B | 855 | 17.270 | 28.206 | 43.171 | 1.00 | 38.24 |
| ATOM | 5198 | CB | ALA | B | 855 | 18.445 | 28.958 | 43.777 | 1.00 | 27.97 |
| ATOM | 5199 | C | ALA | B | 855 | 15.976 | 28.988 | 43.380 | 1.00 | 38.50 |
| ATOM | 5200 | O | ALA | B | 855 | 15.308 | 28.840 | 44.404 | 1.00 | 38.79 |
| ATOM | 5201 | N | LEU | B | 856 | 15.622 | 29.817 | 42.404 | 1.00 | 35.46 |
| ATOM | 5202 | CA | LEU | B | 856 | 14.398 | 30.613 | 42.484 | 1.00 | 35.13 |
| ATOM | 5203 | CB | LEU | B | 856 | 14.507 | 31.858 | 41.597 | 1.00 | 30.40 |
| ATOM | 5204 | CG | LEU | B | 856 | 15.541 | 32.924 | 41.965 | 1.00 | 29.57 |
| ATOM | 5205 | CD1 | LEU | B | 856 | 15.493 | 34.056 | 40.956 | 1.00 | 30.17 |
| ATOM | 5206 | CD2 | LEU | B | 856 | 15.266 | 33.443 | 43.367 | 1.00 | 30.22 |
| ATOM | 5207 | C | LEU | B | 856 | 13.179 | 29.805 | 42.054 | 1.00 | 34.94 |
| ATOM | 5208 | O | LEU | B | 856 | 12.076 | 30.012 | 42.555 | 1.00 | 34.78 |
| ATOM | 5209 | N | ALA | B | 857 | 13.385 | 28.876 | 41.127 | 1.00 | 36.42 |
| ATOM | 5210 | CA | ALA | B | 857 | 12.299 | 28.055 | 40.609 | 1.00 | 36.87 |
| ATOM | 5211 | CB | ALA | B | 857 | 12.648 | 27.577 | 39.206 | 1.00 | 33.84 |
| ATOM | 5212 | C | ALA | B | 857 | 11.926 | 26.859 | 41.482 | 1.00 | 37.18 |
| ATOM | 5213 | O | ALA | B | 857 | 10.774 | 26.429 | 41.482 | 1.00 | 37.43 |
| ATOM | 5214 | N | GLU | B | 858 | 12.892 | 26.319 | 42.216 | 1.00 | 41.64 |
| ATOM | 5215 | CA | GLU | B | 858 | 12.634 | 25.161 | 43.069 | 1.00 | 42.85 |
| ATOM | 5216 | CB | GLU | B | 858 | 13.921 | 24.368 | 43.295 | 1.00 | 56.15 |
| ATOM | 5217 | CG | GLU | B | 858 | 15.034 | 25.173 | 43.930 | 1.00 | 56.44 |
| ATOM | 5218 | CD | GLU | B | 858 | 16.249 | 24.329 | 44.252 | 1.00 | 56.70 |
| ATOM | 5219 | OE1 | GLU | B | 858 | 16.752 | 23.629 | 43.346 | 1.00 | 56.58 |
| ATOM | 5220 | OE2 | GLU | B | 858 | 16.700 | 24.370 | 45.415 | 1.00 | 56.71 |
| ATOM | 5221 | C | GLU | B | 858 | 12.037 | 25.541 | 44.416 | 1.00 | 42.83 |
| ATOM | 5222 | O | GLU | B | 858 | 11.799 | 26.744 | 44.645 | 1.00 | 42.70 |
| ATOM | 5223 | OXT | GLU | B | 858 | 11.814 | 24.618 | 45.227 | 1.00 | 56.34 |
| TER | | | | | | | | | | |
| ATOM | 5225 | N1 | SIL | A | 1 | −13.215 | −5.663 | 26.629 | 1.00 | 24.07 |
| ATOM | 5226 | C18 | SIL | A | 1 | −13.515 | −5.899 | 27.857 | 1.00 | 24.70 |
| ATOM | 5227 | C19 | SIL | A | 1 | −14.726 | −6.742 | 28.146 | 1.00 | 24.74 |
| ATOM | 5228 | C43 | SIL | A | 1 | −15.702 | −6.365 | 29.114 | 1.00 | 25.67 |
| ATOM | 5229 | O44 | SIL | A | 1 | −15.506 | −5.152 | 29.812 | 1.00 | 24.76 |
| ATOM | 5230 | C45 | SIL | A | 1 | −16.389 | −4.626 | 30.824 | 1.00 | 25.28 |
| ATOM | 5231 | C47 | SIL | A | 1 | −15.790 | −3.294 | 31.325 | 1.00 | 25.55 |
| ATOM | 5232 | C41 | SIL | A | 1 | −16.830 | −7.231 | 29.333 | 1.00 | 25.19 |
| ATOM | 5233 | C39 | SIL | A | 1 | −16.955 | −8.443 | 28.594 | 1.00 | 26.15 |
| ATOM | 5234 | C22 | SIL | A | 1 | −15.963 | −8.801 | 27.633 | 1.00 | 25.41 |
| ATOM | 5235 | S23 | SIL | A | 1 | −16.074 | −10.311 | 26.687 | 1.00 | 26.04 |
| ATOM | 5236 | O38 | SIL | A | 1 | −17.300 | −10.973 | 27.102 | 1.00 | 25.69 |
| ATOM | 5237 | O37 | SIL | A | 1 | −14.845 | −11.046 | 26.958 | 1.00 | 26.01 |
| ATOM | 5238 | N24 | SIL | A | 1 | −16.149 | −10.048 | 25.039 | 1.00 | 26.05 |
| ATOM | 5239 | C35 | SIL | A | 1 | −17.168 | −8.998 | 24.670 | 1.00 | 26.28 |
| ATOM | 5240 | C33 | SIL | A | 1 | −17.218 | −8.772 | 23.126 | 1.00 | 26.30 |
| ATOM | 5241 | N29 | SIL | A | 1 | −15.854 | −8.482 | 22.563 | 1.00 | 26.54 |
| ATOM | 5242 | C31 | SIL | A | 1 | −15.924 | −8.260 | 21.089 | 1.00 | 26.02 |
| ATOM | 5243 | C27 | SIL | A | 1 | −14.887 | −9.587 | 22.889 | 1.00 | 26.44 |
| ATOM | 5244 | C25 | SIL | A | 1 | −14.787 | −9.831 | 24.428 | 1.00 | 25.61 |
| ATOM | 5245 | C20 | SIL | A | 1 | −14.868 | −7.946 | 27.427 | 1.00 | 25.44 |
| ATOM | 5246 | N17 | SIL | A | 1 | −12.749 | −5.402 | 28.877 | 1.00 | 24.74 |
| ATOM | 5247 | C15 | SIL | A | 1 | −11.635 | −4.635 | 28.662 | 1.00 | 25.23 |
| ATOM | 5248 | O16 | SIL | A | 1 | −10.964 | −4.202 | 29.580 | 1.00 | 24.95 |
| ATOM | 5249 | C14 | SIL | A | 1 | −11.266 | −4.350 | 27.234 | 1.00 | 25.33 |
| ATOM | 5250 | N11 | SIL | A | 1 | −10.297 | −3.680 | 26.649 | 1.00 | 24.67 |
| ATOM | 5251 | C12 | SIL | A | 1 | −9.206 | −2.961 | 27.299 | 1.00 | 25.39 |
| ATOM | 5252 | N10 | SIL | A | 1 | −10.467 | −3.764 | 25.300 | 1.00 | 25.03 |
| ATOM | 5253 | C3 | SIL | A | 1 | −11.495 | −4.455 | 25.004 | 1.00 | 24.25 |
| ATOM | 5254 | C4 | SIL | A | 1 | −12.014 | −4.783 | 23.619 | 1.00 | 25.73 |
| ATOM | 5255 | C6 | SIL | A | 1 | −12.905 | −3.666 | 22.952 | 1.00 | 26.42 |
| ATOM | 5256 | C8 | SIL | A | 1 | −13.128 | −2.379 | 23.800 | 1.00 | 25.69 |
| ATOM | 5257 | C2 | SIL | A | 1 | −12.059 | −4.866 | 26.277 | 1.00 | 24.93 |
| TER | | | | | | | | | | |
| ATOM | 5259 | N1 | SIL | B | 1 | 15.164 | 33.812 | 25.541 | 1.00 | 25.10 |
| ATOM | 5260 | C18 | SIL | B | 1 | 14.895 | 33.565 | 26.769 | 1.00 | 24.27 |
| ATOM | 5261 | C19 | SIL | B | 1 | 13.700 | 32.702 | 27.070 | 1.00 | 24.84 |
| ATOM | 5262 | C43 | SIL | B | 1 | 12.729 | 33.045 | 28.058 | 1.00 | 24.56 |
| ATOM | 5263 | O44 | SIL | B | 1 | 12.899 | 34.250 | 28.780 | 1.00 | 26.57 |
| ATOM | 5264 | C45 | SIL | B | 1 | 12.006 | 34.722 | 29.810 | 1.00 | 25.69 |
| ATOM | 5265 | C47 | SIL | B | 1 | 12.558 | 36.064 | 30.336 | 1.00 | 25.09 |
| ATOM | 5266 | C41 | SIL | B | 1 | 11.622 | 32.157 | 28.280 | 1.00 | 25.59 |
| ATOM | 5267 | C39 | SIL | B | 1 | 11.507 | 30.960 | 27.526 | 1.00 | 25.77 |
| ATOM | 5268 | C22 | SIL | B | 1 | 12.487 | 30.633 | 26.544 | 1.00 | 25.79 |
| ATOM | 5269 | S23 | SIL | B | 1 | 12.368 | 29.137 | 25.587 | 1.00 | 28.36 |
| ATOM | 5270 | O38 | SIL | B | 1 | 11.164 | 28.457 | 26.018 | 1.00 | 26.14 |
| ATOM | 5271 | O37 | SIL | B | 1 | 13.594 | 28.402 | 25.827 | 1.00 | 26.31 |
| ATOM | 5272 | N24 | SIL | B | 1 | 12.244 | 29.398 | 23.942 | 1.00 | 26.57 |
| ATOM | 5273 | C35 | SIL | B | 1 | 11.267 | 30.495 | 23.609 | 1.00 | 25.93 |
| ATOM | 5274 | C33 | SIL | B | 1 | 11.168 | 30.716 | 22.066 | 1.00 | 26.61 |
| ATOM | 5275 | N29 | SIL | B | 1 | 12.519 | 30.913 | 21.439 | 1.00 | 26.95 |
| ATOM | 5276 | C31 | SIL | B | 1 | 12.402 | 31.132 | 19.969 | 1.00 | 25.72 |
| ATOM | 5277 | C27 | SIL | B | 1 | 13.436 | 29.763 | 21.739 | 1.00 | 26.60 |
| ATOM | 5278 | C25 | SIL | B | 1 | 13.583 | 29.525 | 23.276 | 1.00 | 26.54 |
| ATOM | 5279 | C20 | SIL | B | 1 | 13.569 | 31.507 | 26.332 | 1.00 | 24.95 |
| ATOM | 5280 | N17 | SIL | B | 1 | 15.678 | 34.063 | 27.775 | 1.00 | 25.01 |
| ATOM | 5281 | C15 | SIL | B | 1 | 16.778 | 34.843 | 27.541 | 1.00 | 25.62 |
| ATOM | 5282 | O16 | SIL | B | 1 | 17.471 | 35.279 | 28.442 | 1.00 | 25.56 |
| ATOM | 5283 | C14 | SIL | B | 1 | 17.109 | 35.142 | 26.111 | 1.00 | 24.85 |
| ATOM | 5284 | N11 | SIL | B | 1 | 18.054 | 35.828 | 25.516 | 1.00 | 24.72 |
| ATOM | 5285 | C12 | SIL | B | 1 | 19.147 | 36.556 | 26.153 | 1.00 | 25.78 |
| ATOM | 5286 | N10 | SIL | B | 1 | 17.863 | 35.755 | 24.172 | 1.00 | 25.21 |
| ATOM | 5287 | C3 | SIL | B | 1 | 16.841 | 35.056 | 23.885 | 1.00 | 25.43 |
| ATOM | 5288 | C4 | SIL | B | 1 | 16.304 | 34.734 | 22.500 | 1.00 | 25.15 |
| ATOM | 5289 | C6 | SIL | B | 1 | 15.454 | 35.880 | 21.819 | 1.00 | 26.50 |
| ATOM | 5290 | C8 | SIL | B | 1 | 15.284 | 37.187 | 22.646 | 1.00 | 26.25 |
| ATOM | 5291 | C2 | SIL | B | 1 | 16.305 | 34.627 | 25.167 | 1.00 | 24.69 |
| TER | | | | | | | | | | |
| ATOM | 5293 | ZN | ZN | A | 864 | −8.302 | 0.061 | 19.726 | 1.00 | 33.93 |
| ATOM | 5294 | MG | MG | A | 865 | −10.882 | −0.915 | 17.074 | 1.00 | 20.57 |
| TER | | | | | | | | | | |
| ATOM | 5296 | ZN | ZN | B | 864 | 19.969 | 39.499 | 18.508 | 1.00 | 25.29 |
| ATOM | 5297 | MG | MG | B | 865 | 0.014 | 0.254 | −1.283 | 1.00 | 32.82 |
| END | | | | | | | | | | |

TABLE 4
Atomic coordinates for PDE5* apo protein
Atom Number, Atom Type, Residue Type, Residue Number
Cartesian Coordinates X, Y, Z, Atom Occupancy (O),
Temperature Factor (B)

| | | | | | | X | Y | Z | O | B |
|---|---|---|---|---|---|---|---|---|---|---|
| ATOM | 1 | CB | GLU | A | 536 | 9.887 | 25.182 | 24.787 | 1.00 | 33.98 |
| ATOM | 2 | CG | GLU | A | 536 | 11.305 | 25.697 | 24.635 | 1.00 | 42.32 |
| ATOM | 3 | CD | GLU | A | 536 | 11.492 | 27.074 | 25.245 | 1.00 | 47.12 |
| ATOM | 4 | OE1 | GLU | A | 536 | 11.290 | 27.223 | 26.470 | 1.00 | 54.45 |
| ATOM | 5 | OE2 | GLU | A | 536 | 11.842 | 28.012 | 24.499 | 1.00 | 51.15 |
| ATOM | 6 | C | GLU | A | 536 | 7.942 | 25.044 | 26.327 | 1.00 | 37.22 |
| ATOM | 7 | O | GLU | A | 536 | 7.222 | 24.272 | 25.690 | 1.00 | 36.76 |
| ATOM | 8 | N | GLU | A | 536 | 9.991 | 23.667 | 26.752 | 1.00 | 36.93 |
| ATOM | 9 | CA | GLU | A | 536 | 9.456 | 24.961 | 26.239 | 1.00 | 37.47 |
| ATOM | 10 | N | THR | A | 537 | 7.463 | 25.990 | 27.125 | 1.00 | 37.34 |
| ATOM | 11 | CA | THR | A | 537 | 6.035 | 26.189 | 27.289 | 1.00 | 36.78 |
| ATOM | 12 | CB | THR | A | 537 | 5.750 | 27.235 | 28.387 | 1.00 | 43.61 |
| ATOM | 13 | OG1 | THR | A | 537 | 6.425 | 28.457 | 28.069 | 1.00 | 45.23 |
| ATOM | 14 | CG2 | THR | A | 537 | 6.241 | 26.731 | 29.739 | 1.00 | 34.90 |
| ATOM | 15 | C | THR | A | 537 | 5.452 | 26.660 | 25.957 | 1.00 | 35.15 |
| ATOM | 16 | O | THR | A | 537 | 4.239 | 26.661 | 25.764 | 1.00 | 34.30 |
| ATOM | 17 | N | ARG | A | 538 | 6.336 | 27.046 | 25.040 | 1.00 | 32.72 |
| ATOM | 18 | CA | ARG | A | 538 | 5.943 | 27.514 | 23.712 | 1.00 | 31.35 |
| ATOM | 19 | CB | ARG | A | 538 | 7.143 | 28.133 | 23.002 | 1.00 | 33.78 |
| ATOM | 20 | CG | ARG | A | 538 | 6.910 | 28.452 | 21.531 | 1.00 | 42.77 |
| ATOM | 21 | CD | ARG | A | 538 | 6.041 | 29.690 | 21.340 | 1.00 | 51.85 |
| ATOM | 22 | NE | ARG | A | 538 | 4.638 | 29.475 | 21.687 | 1.00 | 50.21 |
| ATOM | 23 | CZ | ARG | A | 538 | 3.715 | 30.433 | 21.672 | 1.00 | 49.57 |
| ATOM | 24 | NH1 | ARG | A | 538 | 4.047 | 31.673 | 21.330 | 1.00 | 48.08 |
| ATOM | 25 | NH2 | ARG | A | 538 | 2.458 | 30.154 | 21.992 | 1.00 | 49.14 |
| ATOM | 26 | C | ARG | A | 538 | 5.405 | 26.369 | 22.861 | 1.00 | 30.33 |
| ATOM | 27 | O | ARG | A | 538 | 4.381 | 26.501 | 22.187 | 1.00 | 28.72 |
| ATOM | 28 | N | GLU | A | 539 | 6.120 | 25.250 | 22.876 | 1.00 | 28.94 |
| ATOM | 29 | CA | GLU | A | 539 | 5.709 | 24.080 | 22.115 | 1.00 | 25.47 |
| ATOM | 30 | CB | GLU | A | 539 | 6.807 | 23.024 | 22.141 | 1.00 | 30.35 |
| ATOM | 31 | CG | GLU | A | 539 | 8.055 | 23.426 | 21.391 | 1.00 | 31.75 |
| ATOM | 32 | CD | GLU | A | 539 | 9.188 | 22.463 | 21.628 | 1.00 | 31.48 |
| ATOM | 33 | OE1 | GLU | A | 539 | 9.689 | 22.410 | 22.771 | 1.00 | 46.55 |
| ATOM | 34 | OE2 | GLU | A | 539 | 9.575 | 21.754 | 20.676 | 1.00 | 57.06 |
| ATOM | 35 | C | GLU | A | 539 | 4.437 | 23.511 | 22.718 | 1.00 | 23.49 |
| ATOM | 36 | O | GLU | A | 539 | 3.599 | 22.952 | 22.011 | 1.00 | 21.01 |
| ATOM | 37 | N | LEU | A | 540 | 4.299 | 23.653 | 24.032 | 1.00 | 22.37 |
| ATOM | 38 | CA | LEU | A | 540 | 3.114 | 23.159 | 24.713 | 1.00 | 22.10 |
| ATOM | 39 | CB | LEU | A | 540 | 3.302 | 23.223 | 26.231 | 1.00 | 19.44 |
| ATOM | 40 | CG | LEU | A | 540 | 2.098 | 22.763 | 27.059 | 1.00 | 22.04 |
| ATOM | 41 | CD1 | LEU | A | 540 | 1.686 | 21.365 | 26.627 | 1.00 | 19.81 |
| ATOM | 42 | CD2 | LEU | A | 540 | 2.441 | 22.783 | 28.539 | 1.00 | 38.12 |
| ATOM | 43 | C | LEU | A | 540 | 1.907 | 23.995 | 24.300 | 1.00 | 21.23 |
| ATOM | 44 | O | LEU | A | 540 | 0.844 | 23.461 | 23.990 | 1.00 | 19.37 |
| ATOM | 45 | N | GLN | A | 541 | 2.074 | 25.313 | 24.283 | 1.00 | 20.36 |
| ATOM | 46 | CA | GLN | A | 541 | 0.972 | 26.185 | 23.905 | 1.00 | 18.42 |
| ATOM | 47 | CB | GLN | A | 541 | 1.410 | 27.651 | 23.966 | 1.00 | 20.67 |
| ATOM | 48 | CG | GLN | A | 541 | 1.873 | 28.081 | 25.350 | 1.00 | 24.12 |
| ATOM | 49 | CD | GLN | A | 541 | 2.356 | 29.519 | 25.401 | 1.00 | 24.37 |
| ATOM | 50 | OE1 | GLN | A | 541 | 3.047 | 29.986 | 24.497 | 1.00 | 17.86 |
| ATOM | 51 | NE2 | GLN | A | 541 | 2.008 | 30.223 | 26.474 | 1.00 | 20.12 |
| ATOM | 52 | C | GLN | A | 541 | 0.477 | 25.833 | 22.505 | 1.00 | 17.74 |
| ATOM | 53 | O | GLN | A | 541 | −0.726 | 25.799 | 22.254 | 1.00 | 19.08 |
| ATOM | 54 | N | SER | A | 542 | 1.404 | 25.552 | 21.596 | 1.00 | 15.82 |
| ATOM | 55 | CA | SER | A | 542 | 1.026 | 25.207 | 20.231 | 1.00 | 18.42 |
| ATOM | 56 | CB | SER | A | 542 | 2.263 | 25.179 | 19.329 | 1.00 | 20.91 |
| ATOM | 57 | OG | SER | A | 542 | 2.857 | 26.463 | 19.251 | 1.00 | 37.22 |
| ATOM | 58 | C | SER | A | 542 | 0.320 | 23.858 | 20.174 | 1.00 | 16.82 |
| ATOM | 59 | O | SER | A | 542 | −0.710 | 23.709 | 19.520 | 1.00 | 17.71 |
| ATOM | 60 | N | LEU | A | 543 | 0.875 | 22.874 | 20.872 | 1.00 | 17.64 |
| ATOM | 61 | CA | LEU | A | 543 | 0.297 | 21.538 | 20.878 | 1.00 | 17.88 |
| ATOM | 62 | CB | LEU | A | 543 | 1.269 | 20.550 | 21.531 | 1.00 | 13.54 |
| ATOM | 63 | CG | LEU | A | 543 | 0.762 | 19.113 | 21.695 | 1.00 | 17.02 |
| ATOM | 64 | CD1 | LEU | A | 543 | 0.501 | 18.504 | 20.329 | 1.00 | 14.15 |
| ATOM | 65 | CD2 | LEU | A | 543 | 1.789 | 18.284 | 22.465 | 1.00 | 10.59 |
| ATOM | 66 | C | LEU | A | 543 | −1.049 | 21.468 | 21.592 | 1.00 | 18.33 |
| ATOM | 67 | O | LEU | A | 543 | −2.013 | 20.914 | 21.064 | 1.00 | 19.18 |
| ATOM | 68 | N | ALA | A | 544 | −1.112 | 22.038 | 22.790 | 1.00 | 18.60 |
| ATOM | 69 | CA | ALA | A | 544 | −2.332 | 22.003 | 23.588 | 1.00 | 19.09 |
| ATOM | 70 | CB | ALA | A | 544 | −2.053 | 22.573 | 24.972 | 1.00 | 17.83 |
| ATOM | 71 | C | ALA | A | 544 | −3.527 | 22.717 | 22.967 | 1.00 | 21.56 |
| ATOM | 72 | O | ALA | A | 544 | −4.667 | 22.306 | 23.165 | 1.00 | 23.51 |
| ATOM | 73 | N | ALA | A | 545 | −3.269 | 23.782 | 22.217 | 1.00 | 23.98 |
| ATOM | 74 | CA | ALA | A | 545 | −4.348 | 24.547 | 21.599 | 1.00 | 24.34 |
| ATOM | 75 | CB | ALA | A | 545 | −3.861 | 25.952 | 21.274 | 1.00 | 26.53 |
| ATOM | 76 | C | ALA | A | 545 | −4.901 | 23.891 | 20.341 | 1.00 | 25.76 |
| ATOM | 77 | O | ALA | A | 545 | −6.065 | 24.079 | 19.992 | 1.00 | 28.16 |
| ATOM | 78 | N | ALA | A | 546 | −4.059 | 23.115 | 19.668 | 1.00 | 24.74 |
| ATOM | 79 | CA | ALA | A | 546 | −4.441 | 22.443 | 18.430 | 1.00 | 24.22 |
| ATOM | 80 | CB | ALA | A | 546 | −3.239 | 21.701 | 17.861 | 1.00 | 22.00 |
| ATOM | 81 | C | ALA | A | 546 | −5.621 | 21.485 | 18.529 | 1.00 | 21.89 |
| ATOM | 82 | O | ALA | A | 546 | −5.803 | 20.791 | 19.529 | 1.00 | 23.27 |
| ATOM | 83 | N | VAL | A | 547 | −6.433 | 21.464 | 17.478 | 1.00 | 21.72 |
| ATOM | 84 | CA | VAL | A | 547 | −7.560 | 20.548 | 17.418 | 1.00 | 21.65 |
| ATOM | 85 | CB | VAL | A | 547 | −8.660 | 21.045 | 16.454 | 1.00 | 23.33 |
| ATOM | 86 | CG1 | VAL | A | 547 | −9.754 | 19.993 | 16.326 | 1.00 | 25.11 |
| ATOM | 87 | CG2 | VAL | A | 547 | −9.250 | 22.355 | 16.965 | 1.00 | 23.58 |
| ATOM | 88 | C | VAL | A | 547 | −6.925 | 19.284 | 16.850 | 1.00 | 22.28 |
| ATOM | 89 | O | VAL | A | 547 | −6.280 | 19.329 | 15.805 | 1.00 | 24.96 |
| ATOM | 90 | N | VAL | A | 548 | −7.090 | 18.165 | 17.542 | 1.00 | 21.82 |
| ATOM | 91 | CA | VAL | A | 548 | −6.498 | 16.909 | 17.098 | 1.00 | 19.25 |
| ATOM | 92 | CB | VAL | A | 548 | −6.387 | 15.925 | 18.278 | 1.00 | 19.56 |
| ATOM | 93 | CG1 | VAL | A | 548 | −5.669 | 14.659 | 17.840 | 1.00 | 23.78 |
| ATOM | 94 | CG2 | VAL | A | 548 | −5.656 | 16.593 | 19.438 | 1.00 | 24.95 |
| ATOM | 95 | C | VAL | A | 548 | −7.284 | 16.241 | 15.975 | 1.00 | 17.26 |
| ATOM | 96 | O | VAL | A | 548 | −8.453 | 15.904 | 16.146 | 1.00 | 19.24 |
| ATOM | 97 | N | PRO | A | 549 | −6.646 | 16.038 | 14.808 | 1.00 | 17.33 |
| ATOM | 98 | CD | PRO | A | 549 | −5.293 | 16.478 | 14.431 | 1.00 | 18.64 |
| ATOM | 99 | CA | PRO | A | 549 | −7.315 | 15.401 | 13.670 | 1.00 | 19.43 |
| ATOM | 100 | CB | PRO | A | 549 | −6.245 | 15.425 | 12.575 | 1.00 | 17.30 |
| ATOM | 101 | CG | PRO | A | 549 | −5.420 | 16.615 | 12.931 | 1.00 | 23.24 |
| ATOM | 102 | C | PRO | A | 549 | −7.742 | 13.982 | 14.023 | 1.00 | 20.23 |
| ATOM | 103 | O | PRO | A | 549 | −7.259 | 13.402 | 15.001 | 1.00 | 16.82 |
| ATOM | 104 | N | SER | A | 550 | −8.645 | 13.431 | 13.220 | 1.00 | 17.57 |
| ATOM | 105 | CA | SER | A | 550 | −9.153 | 12.081 | 13.432 | 1.00 | 20.68 |
| ATOM | 106 | CB | SER | A | 550 | −10.343 | 11.822 | 12.512 | 1.00 | 17.19 |
| ATOM | 107 | OG | SER | A | 550 | −9.934 | 11.861 | 11.153 | 1.00 | 18.60 |
| ATOM | 108 | C | SER | A | 550 | −8.075 | 11.055 | 13.127 | 1.00 | 17.34 |
| ATOM | 109 | O | SER | A | 550 | −7.096 | 11.357 | 12.443 | 1.00 | 16.71 |
| ATOM | 110 | N | ALA | A | 551 | −8.257 | 9.841 | 13.637 | 1.00 | 18.46 |
| ATOM | 111 | CA | ALA | A | 551 | −7.304 | 8.769 | 13.380 | 1.00 | 19.34 |
| ATOM | 112 | CB | ALA | A | 551 | −7.706 | 7.507 | 14.143 | 1.00 | 17.06 |
| ATOM | 113 | C | ALA | A | 551 | −7.322 | 8.502 | 11.879 | 1.00 | 18.62 |
| ATOM | 114 | O | ALA | A | 551 | −6.284 | 8.262 | 11.261 | 1.00 | 20.73 |
| ATOM | 115 | N | GLN | A | 552 | −8.518 | 8.554 | 11.300 | 1.00 | 19.78 |
| ATOM | 116 | CA | GLN | A | 552 | −8.700 | 8.327 | 9.869 | 1.00 | 21.36 |
| ATOM | 117 | CB | GLN | A | 552 | −10.175 | 8.528 | 9.502 | 1.00 | 23.40 |
| ATOM | 118 | CG | GLN | A | 552 | −10.541 | 8.181 | 8.067 | 1.00 | 31.79 |
| ATOM | 119 | CD | GLN | A | 552 | −12.026 | 8.358 | 7.797 | 1.00 | 35.80 |
| ATOM | 120 | OE1 | GLN | A | 552 | −12.864 | 7.675 | 8.390 | 1.00 | 46.36 |
| ATOM | 121 | NE2 | GLN | A | 552 | −12.358 | 9.283 | 6.902 | 1.00 | 49.03 |
| ATOM | 122 | C | GLN | A | 552 | −7.820 | 9.301 | 9.086 | 1.00 | 21.35 |
| ATOM | 123 | O | GLN | A | 552 | −7.040 | 8.897 | 8.219 | 1.00 | 21.48 |
| ATOM | 124 | N | THR | A | 553 | −7.938 | 10.584 | 9.408 | 1.00 | 19.75 |
| ATOM | 125 | CA | THR | A | 553 | −7.151 | 11.612 | 8.739 | 1.00 | 19.52 |
| ATOM | 126 | CB | THR | A | 553 | −7.501 | 13.017 | 9.268 | 1.00 | 18.18 |
| ATOM | 127 | OG1 | THR | A | 553 | −8.871 | 13.318 | 8.971 | 1.00 | 19.96 |
| ATOM | 128 | CG2 | THR | A | 553 | −6.597 | 14.066 | 8.627 | 1.00 | 18.92 |
| ATOM | 129 | C | THR | A | 553 | −5.654 | 11.396 | 8.926 | 1.00 | 20.00 |
| ATOM | 130 | O | THR | A | 553 | −4.875 | 11.523 | 7.980 | 1.00 | 19.53 |
| ATOM | 131 | N | LEU | A | 554 | −5.260 | 11.062 | 10.150 | 1.00 | 19.10 |
| ATOM | 132 | CA | LEU | A | 554 | −3.855 | 10.853 | 10.474 | 1.00 | 20.03 |
| ATOM | 133 | CB | LEU | A | 554 | −3.650 | 10.974 | 11.987 | 1.00 | 17.91 |
| ATOM | 134 | CG | LEU | A | 554 | −4.091 | 12.323 | 12.571 | 1.00 | 18.40 |
| ATOM | 135 | CD1 | LEU | A | 554 | −3.972 | 12.316 | 14.082 | 1.00 | 16.11 |
| ATOM | 136 | CD2 | LEU | A | 554 | −3.237 | 13.436 | 11.977 | 1.00 | 23.68 |
| ATOM | 137 | C | LEU | A | 554 | −3.303 | 9.521 | 9.970 | 1.00 | 19.88 |
| ATOM | 138 | O | LEU | A | 554 | −2.093 | 9.304 | 9.994 | 1.00 | 19.74 |
| ATOM | 139 | N | LYS | A | 555 | −4.198 | 8.637 | 9.533 | 1.00 | 17.37 |
| ATOM | 140 | CA | LYS | A | 555 | −3.833 | 7.327 | 8.997 | 1.00 | 19.69 |
| ATOM | 141 | CB | LYS | A | 555 | −3.032 | 7.523 | 7.708 | 1.00 | 26.45 |
| ATOM | 142 | CG | LYS | A | 555 | −3.740 | 8.429 | 6.708 | 1.00 | 33.54 |
| ATOM | 143 | CD | LYS | A | 555 | −2.829 | 8.837 | 5.565 | 1.00 | 36.95 |
| ATOM | 144 | CE | LYS | A | 555 | −3.542 | 9.803 | 4.633 | 1.00 | 36.53 |
| ATOM | 145 | NZ | LYS | A | 555 | −2.645 | 10.308 | 3.562 | 1.00 | 43.43 |
| ATOM | 146 | C | LYS | A | 555 | −3.043 | 6.454 | 9.976 | 1.00 | 19.45 |
| ATOM | 147 | O | LYS | A | 555 | −2.294 | 5.569 | 9.565 | 1.00 | 18.29 |
| ATOM | 148 | N | ILE | A | 556 | −3.227 | 6.688 | 11.271 | 1.00 | 17.06 |
| ATOM | 149 | CA | ILE | A | 556 | −2.501 | 5.917 | 12.273 | 1.00 | 16.83 |
| ATOM | 150 | CB | ILE | A | 556 | −2.475 | 6.638 | 13.641 | 1.00 | 13.95 |
| ATOM | 151 | CG2 | ILE | A | 556 | −1.565 | 7.856 | 13.569 | 1.00 | 16.87 |
| ATOM | 152 | CG1 | ILE | A | 556 | −3.897 | 7.017 | 14.062 | 1.00 | 14.93 |
| ATOM | 153 | CD1 | ILE | A | 556 | −3.981 | 7.598 | 15.460 | 1.00 | 19.73 |
| ATOM | 154 | C | ILE | A | 556 | −3.019 | 4.496 | 12.489 | 1.00 | 14.60 |
| ATOM | 155 | O | ILE | A | 556 | −2.443 | 3.745 | 13.271 | 1.00 | 12.74 |
| ATOM | 156 | N | THR | A | 557 | −4.099 | 4.125 | 11.806 | 1.00 | 14.27 |
| ATOM | 157 | CA | THR | A | 557 | −4.653 | 2.776 | 11.938 | 1.00 | 15.05 |
| ATOM | 158 | CB | THR | A | 557 | −6.187 | 2.772 | 11.699 | 1.00 | 21.22 |
| ATOM | 159 | OG1 | THR | A | 557 | −6.813 | 3.726 | 12.567 | 1.00 | 20.77 |
| ATOM | 160 | CG2 | THR | A | 557 | −6.773 | 1.398 | 11.982 | 1.00 | 21.26 |
| ATOM | 161 | C | THR | A | 557 | −3.991 | 1.855 | 10.907 | 1.00 | 14.39 |
| ATOM | 162 | O | THR | A | 557 | −4.131 | 0.631 | 10.955 | 1.00 | 16.89 |
| ATOM | 163 | N | ASP | A | 558 | −3.257 | 2.456 | 9.979 | 1.00 | 14.56 |
| ATOM | 164 | CA | ASP | A | 558 | −2.593 | 1.702 | 8.923 | 1.00 | 14.77 |
| ATOM | 165 | CB | ASP | A | 558 | −2.374 | 2.613 | 7.713 | 1.00 | 20.77 |
| ATOM | 166 | CG | ASP | A | 558 | −3.648 | 3.312 | 7.274 | 1.00 | 30.09 |
| ATOM | 167 | OD1 | ASP | A | 558 | −4.657 | 2.616 | 7.031 | 1.00 | 38.75 |
| ATOM | 168 | OD2 | ASP | A | 558 | −3.642 | 4.555 | 7.173 | 1.00 | 46.69 |
| ATOM | 169 | C | ASP | A | 558 | −1.258 | 1.086 | 9.351 | 1.00 | 14.68 |
| ATOM | 170 | O | ASP | A | 558 | −0.365 | 1.787 | 9.829 | 1.00 | 13.87 |
| ATOM | 171 | N | PHE | A | 559 | −1.124 | −0.227 | 9.175 | 1.00 | 13.82 |
| ATOM | 172 | CA | PHE | A | 559 | 0.119 | −0.916 | 9.526 | 1.00 | 13.07 |
| ATOM | 173 | CB | PHE | A | 559 | 0.000 | −2.427 | 9.283 | 1.00 | 14.41 |
| ATOM | 174 | CG | PHE | A | 559 | −0.732 | −3.175 | 10.366 | 1.00 | 18.21 |
| ATOM | 175 | CD1 | PHE | A | 559 | −0.289 | −3.134 | 11.680 | 1.00 | 14.30 |
| ATOM | 176 | CD2 | PHE | A | 559 | −1.836 | −3.960 | 10.060 | 1.00 | 16.61 |
| ATOM | 177 | CE1 | PHE | A | 559 | −0.931 | −3.866 | 12.668 | 1.00 | 15.41 |
| ATOM | 178 | CE2 | PHE | A | 559 | −2.482 | −4.692 | 11.043 | 1.00 | 15.41 |
| ATOM | 179 | CZ | PHE | A | 559 | −2.031 | −4.647 | 12.344 | 1.00 | 12.10 |
| ATOM | 180 | C | PHE | A | 559 | 1.277 | −0.386 | 8.680 | 1.00 | 11.71 |
| ATOM | 181 | O | PHE | A | 559 | 2.431 | −0.411 | 9.109 | 1.00 | 12.25 |
| ATOM | 182 | N | SER | A | 560 | 0.965 | 0.079 | 7.473 | 1.00 | 13.82 |
| ATOM | 183 | CA | SER | A | 560 | 1.979 | 0.597 | 6.555 | 1.00 | 13.86 |
| ATOM | 184 | CB | SER | A | 560 | 1.538 | 0.384 | 5.106 | 1.00 | 19.52 |
| ATOM | 185 | OG | SER | A | 560 | 1.411 | −0.996 | 4.816 | 1.00 | 19.35 |
| ATOM | 186 | C | SER | A | 560 | 2.294 | 2.071 | 6.770 | 1.00 | 12.88 |
| ATOM | 187 | O | SER | A | 560 | 2.959 | 2.698 | 5.945 | 1.00 | 14.29 |
| ATOM | 188 | N | PHE | A | 561 | 1.815 | 2.608 | 7.886 | 1.00 | 12.82 |
| ATOM | 189 | CA | PHE | A | 561 | 2.021 | 4.009 | 8.261 | 1.00 | 13.63 |
| ATOM | 190 | CB | PHE | A | 561 | 1.567 | 4.207 | 9.713 | 1.00 | 14.59 |
| ATOM | 191 | CG | PHE | A | 561 | 1.835 | 5.583 | 10.258 | 1.00 | 12.08 |
| ATOM | 192 | CD1 | PHE | A | 561 | 0.956 | 6.625 | 10.014 | 1.00 | 17.74 |
| ATOM | 193 | CD2 | PHE | A | 561 | 2.967 | 5.829 | 11.022 | 1.00 | 10.31 |
| ATOM | 194 | CE1 | PHE | A | 561 | 1.199 | 7.891 | 10.524 | 1.00 | 21.75 |
| ATOM | 195 | CE2 | PHE | A | 561 | 3.220 | 7.093 | 11.537 | 1.00 | 13.27 |
| ATOM | 196 | CZ | PHE | A | 561 | 2.333 | 8.126 | 11.287 | 1.00 | 18.85 |
| ATOM | 197 | C | PHE | A | 561 | 3.466 | 4.498 | 8.125 | 1.00 | 14.38 |
| ATOM | 198 | O | PHE | A | 561 | 4.413 | 3.791 | 8.485 | 1.00 | 11.10 |
| ATOM | 199 | N | SER | A | 562 | 3.625 | 5.716 | 7.607 | 1.00 | 14.38 |
| ATOM | 200 | CA | SER | A | 562 | 4.939 | 6.334 | 7.453 | 1.00 | 13.72 |
| ATOM | 201 | CB | SER | A | 562 | 5.283 | 6.520 | 5.972 | 1.00 | 15.96 |
| ATOM | 202 | OG | SER | A | 562 | 6.542 | 7.159 | 5.835 | 1.00 | 23.25 |
| ATOM | 203 | C | SER | A | 562 | 4.940 | 7.695 | 8.159 | 1.00 | 12.53 |
| ATOM | 204 | O | SER | A | 562 | 4.009 | 8.490 | 8.014 | 1.00 | 12.55 |
| ATOM | 205 | N | ASP | A | 563 | 5.992 | 7.965 | 8.922 | 1.00 | 13.45 |
| ATOM | 206 | CA | ASP | A | 563 | 6.094 | 9.223 | 9.661 | 1.00 | 15.79 |
| ATOM | 207 | CB | ASP | A | 563 | 6.662 | 8.953 | 11.050 | 1.00 | 13.73 |
| ATOM | 208 | CG | ASP | A | 563 | 8.166 | 8.765 | 11.022 | 1.00 | 15.49 |
| ATOM | 209 | OD1 | ASP | A | 563 | 8.895 | 9.680 | 11.465 | 1.00 | 18.81 |
| ATOM | 210 | OD2 | ASP | A | 563 | 8.625 | 7.712 | 10.537 | 1.00 | 16.12 |
| ATOM | 211 | C | ASP | A | 563 | 7.009 | 10.232 | 8.974 | 1.00 | 16.63 |
| ATOM | 212 | O | ASP | A | 563 | 7.112 | 11.383 | 9.404 | 1.00 | 17.82 |
| ATOM | 213 | N | PHE | A | 564 | 7.673 | 9.796 | 7.911 | 1.00 | 16.51 |
| ATOM | 214 | CA | PHE | A | 564 | 8.633 | 10.635 | 7.205 | 1.00 | 18.23 |
| ATOM | 215 | CB | PHE | A | 564 | 9.207 | 9.871 | 6.011 | 1.00 | 15.87 |
| ATOM | 216 | CG | PHE | A | 564 | 10.446 | 10.494 | 5.438 | 1.00 | 26.61 |
| ATOM | 217 | CD1 | PHE | A | 564 | 11.599 | 10.592 | 6.201 | 1.00 | 20.64 |
| ATOM | 218 | CD2 | PHE | A | 564 | 10.458 | 10.991 | 4.144 | 1.00 | 27.32 |
| ATOM | 219 | CE1 | PHE | A | 564 | 12.742 | 11.174 | 5.685 | 1.00 | 30.38 |
| ATOM | 220 | CE2 | PHE | A | 564 | 11.599 | 11.574 | 3.622 | 1.00 | 33.45 |
| ATOM | 221 | CZ | PHE | A | 564 | 12.742 | 11.665 | 4.394 | 1.00 | 33.80 |
| ATOM | 222 | C | PHE | A | 564 | 8.157 | 12.010 | 6.740 | 1.00 | 18.52 |
| ATOM | 223 | O | PHE | A | 564 | 8.942 | 12.959 | 6.728 | 1.00 | 19.54 |
| ATOM | 224 | N | GLU | A | 565 | 6.885 | 12.122 | 6.369 | 1.00 | 16.14 |
| ATOM | 225 | CA | GLU | A | 565 | 6.340 | 13.391 | 5.890 | 1.00 | 19.03 |
| ATOM | 226 | CB | GLU | A | 565 | 5.277 | 13.142 | 4.813 | 1.00 | 18.54 |
| ATOM | 227 | CG | GLU | A | 565 | 5.708 | 12.238 | 3.668 | 1.00 | 33.46 |
| ATOM | 228 | CD | GLU | A | 565 | 6.004 | 10.818 | 4.117 | 1.00 | 40.92 |
| ATOM | 229 | OE1 | GLU | A | 565 | 5.205 | 10.258 | 4.899 | 1.00 | 41.02 |
| ATOM | 230 | OE2 | GLU | A | 565 | 7.032 | 10.259 | 3.680 | 1.00 | 50.71 |
| ATOM | 231 | C | GLU | A | 565 | 5.710 | 14.229 | 7.002 | 1.00 | 19.43 |
| ATOM | 232 | O | GLU | A | 565 | 5.158 | 15.301 | 6.740 | 1.00 | 20.33 |
| ATOM | 233 | N | LEU | A | 566 | 5.786 | 13.746 | 8.238 | 1.00 | 13.91 |
| ATOM | 234 | CA | LEU | A | 566 | 5.193 | 14.463 | 9.362 | 1.00 | 15.26 |
| ATOM | 235 | CB | LEU | A | 566 | 4.515 | 13.482 | 10.321 | 1.00 | 15.63 |
| ATOM | 236 | CG | LEU | A | 566 | 3.329 | 12.669 | 9.799 | 1.00 | 24.63 |
| ATOM | 237 | CD1 | LEU | A | 566 | 2.864 | 11.698 | 10.881 | 1.00 | 23.72 |
| ATOM | 238 | CD2 | LEU | A | 566 | 2.199 | 13.607 | 9.399 | 1.00 | 33.15 |
| ATOM | 239 | C | LEU | A | 566 | 6.187 | 15.306 | 10.147 | 1.00 | 14.43 |
| ATOM | 240 | O | LEU | A | 566 | 7.358 | 14.960 | 10.265 | 1.00 | 17.81 |
| ATOM | 241 | N | SER | A | 567 | 5.699 | 16.419 | 10.685 | 1.00 | 14.78 |
| ATOM | 242 | CA | SER | A | 567 | 6.517 | 17.317 | 11.486 | 1.00 | 14.98 |
| ATOM | 243 | CB | SER | A | 567 | 5.928 | 18.727 | 11.467 | 1.00 | 21.89 |
| ATOM | 244 | OG | SER | A | 567 | 4.679 | 18.752 | 12.144 | 1.00 | 17.63 |
| ATOM | 245 | C | SER | A | 567 | 6.492 | 16.797 | 12.922 | 1.00 | 13.89 |
| ATOM | 246 | O | SER | A | 567 | 5.654 | 15.963 | 13.268 | 1.00 | 13.82 |
| ATOM | 247 | N | ASP | A | 568 | 7.403 | 17.289 | 13.753 | 1.00 | 15.43 |
| ATOM | 248 | CA | ASP | A | 568 | 7.428 | 16.869 | 15.149 | 1.00 | 15.57 |
| ATOM | 249 | CB | ASP | A | 568 | 8.536 | 17.603 | 15.904 | 1.00 | 19.60 |
| ATOM | 250 | CG | ASP | A | 568 | 9.888 | 16.947 | 15.727 | 1.00 | 16.75 |
| ATOM | 251 | OD1 | ASP | A | 568 | 9.989 | 16.021 | 14.899 | 1.00 | 19.05 |
| ATOM | 252 | OD2 | ASP | A | 568 | 10.848 | 17.355 | 16.415 | 1.00 | 23.47 |
| ATOM | 253 | C | ASP | A | 568 | 6.076 | 17.145 | 15.805 | 1.00 | 16.52 |
| ATOM | 254 | O | ASP | A | 568 | 5.559 | 16.313 | 16.556 | 1.00 | 15.97 |
| ATOM | 255 | N | LEU | A | 569 | 5.505 | 18.311 | 15.516 | 1.00 | 15.09 |
| ATOM | 256 | CA | LEU | A | 569 | 4.213 | 18.683 | 16.076 | 1.00 | 15.08 |
| ATOM | 257 | CB | LEU | A | 569 | 3.815 | 20.093 | 15.620 | 1.00 | 16.05 |
| ATOM | 258 | CG | LEU | A | 569 | 2.385 | 20.516 | 15.974 | 1.00 | 23.47 |
| ATOM | 259 | CD1 | LEU | A | 569 | 2.203 | 20.527 | 17.489 | 1.00 | 21.65 |
| ATOM | 260 | CD2 | LEU | A | 569 | 2.103 | 21.896 | 15.398 | 1.00 | 27.54 |
| ATOM | 261 | C | LEU | A | 569 | 3.136 | 17.694 | 15.647 | 1.00 | 12.90 |
| ATOM | 262 | O | LEU | A | 569 | 2.279 | 17.308 | 16.441 | 1.00 | 16.26 |
| ATOM | 263 | N | GLU | A | 570 | 3.184 | 17.281 | 14.386 | 1.00 | 13.40 |
| ATOM | 264 | CA | GLU | A | 570 | 2.202 | 16.340 | 13.869 | 1.00 | 14.08 |
| ATOM | 265 | CB | GLU | A | 570 | 2.354 | 16.196 | 12.352 | 1.00 | 14.43 |
| ATOM | 266 | CG | GLU | A | 570 | 1.885 | 17.432 | 11.579 | 1.00 | 20.85 |
| ATOM | 267 | CD | GLU | A | 570 | 2.062 | 17.291 | 10.081 | 1.00 | 23.63 |
| ATOM | 268 | OE1 | GLU | A | 570 | 3.218 | 17.158 | 9.632 | 1.00 | 21.68 |
| ATOM | 269 | OE2 | GLU | A | 570 | 1.047 | 17.311 | 9.352 | 1.00 | 28.09 |
| ATOM | 270 | C | GLU | A | 570 | 2.309 | 14.978 | 14.547 | 1.00 | 14.43 |
| ATOM | 271 | O | GLU | A | 570 | 1.293 | 14.319 | 14.780 | 1.00 | 15.20 |
| ATOM | 272 | N | THR | A | 571 | 3.529 | 14.551 | 14.866 | 1.00 | 12.67 |
| ATOM | 273 | CA | THR | A | 571 | 3.694 | 13.259 | 15.538 | 1.00 | 12.83 |
| ATOM | 274 | CB | THR | A | 571 | 5.182 | 12.816 | 15.638 | 1.00 | 13.30 |
| ATOM | 275 | OG1 | THR | A | 571 | 5.915 | 13.737 | 16.452 | 1.00 | 12.68 |
| ATOM | 276 | CG2 | THR | A | 571 | 5.817 | 12.738 | 14.250 | 1.00 | 12.38 |
| ATOM | 277 | C | THR | A | 571 | 3.110 | 13.360 | 16.945 | 1.00 | 11.29 |
| ATOM | 278 | O | THR | A | 571 | 2.589 | 12.381 | 17.483 | 1.00 | 12.14 |
| ATOM | 279 | N | ALA | A | 572 | 3.202 | 14.548 | 17.538 | 1.00 | 12.42 |
| ATOM | 280 | CA | ALA | A | 572 | 2.659 | 14.780 | 18.869 | 1.00 | 11.84 |
| ATOM | 281 | CB | ALA | A | 572 | 3.090 | 16.142 | 19.388 | 1.00 | 12.18 |
| ATOM | 282 | C | ALA | A | 572 | 1.136 | 14.701 | 18.803 | 1.00 | 13.34 |
| ATOM | 283 | O | ALA | A | 572 | 0.500 | 14.128 | 19.683 | 1.00 | 11.72 |
| ATOM | 284 | N | LEU | A | 573 | 0.554 | 15.283 | 17.758 | 1.00 | 12.67 |
| ATOM | 285 | CA | LEU | A | 573 | −0.894 | 15.244 | 17.583 | 1.00 | 13.98 |
| ATOM | 286 | CB | LEU | A | 573 | −1.307 | 16.104 | 16.384 | 1.00 | 14.33 |
| ATOM | 287 | CG | LEU | A | 573 | −1.135 | 17.619 | 16.544 | 1.00 | 14.55 |
| ATOM | 288 | CD1 | LEU | A | 573 | −1.445 | 18.310 | 15.224 | 1.00 | 21.57 |
| ATOM | 289 | CD2 | LEU | A | 573 | −2.061 | 18.125 | 17.647 | 1.00 | 20.27 |
| ATOM | 290 | C | LEU | A | 573 | −1.338 | 13.795 | 17.366 | 1.00 | 15.26 |
| ATOM | 291 | O | LEU | A | 573 | −2.394 | 13.378 | 17.853 | 1.00 | 15.01 |
| ATOM | 292 | N | CYS | A | 574 | −0.534 | 13.030 | 16.632 | 1.00 | 13.44 |
| ATOM | 293 | CA | CYS | A | 574 | −0.852 | 11.625 | 16.385 | 1.00 | 13.06 |
| ATOM | 294 | CB | CYS | A | 574 | 0.187 | 10.974 | 15.470 | 1.00 | 12.27 |
| ATOM | 295 | SG | CYS | A | 574 | 0.049 | 11.382 | 13.714 | 1.00 | 15.24 |
| ATOM | 296 | C | CYS | A | 574 | −0.873 | 10.864 | 17.703 | 1.00 | 12.15 |
| ATOM | 297 | O | CYS | A | 574 | −1.710 | 9.991 | 17.919 | 1.00 | 13.51 |
| ATOM | 298 | N | THR | A | 575 | 0.066 | 11.194 | 18.581 | 1.00 | 11.60 |
| ATOM | 299 | CA | THR | A | 575 | 0.158 | 10.532 | 19.872 | 1.00 | 12.28 |
| ATOM | 300 | CB | THR | A | 575 | 1.453 | 10.949 | 20.592 | 1.00 | 10.90 |
| ATOM | 301 | OG1 | THR | A | 575 | 2.575 | 10.576 | 19.777 | 1.00 | 13.47 |
| ATOM | 302 | CG2 | THR | A | 575 | 1.565 | 10.264 | 21.944 | 1.00 | 10.06 |
| ATOM | 303 | C | THR | A | 575 | −1.068 | 10.841 | 20.726 | 1.00 | 11.74 |
| ATOM | 304 | O | THR | A | 575 | −1.613 | 9.952 | 21.379 | 1.00 | 10.78 |
| ATOM | 305 | N | ILE | A | 576 | −1.514 | 12.095 | 20.717 | 1.00 | 12.29 |
| ATOM | 306 | CA | ILE | A | 576 | −2.700 | 12.453 | 21.484 | 1.00 | 11.92 |
| ATOM | 307 | CB | ILE | A | 576 | −3.046 | 13.945 | 21.354 | 1.00 | 11.38 |
| ATOM | 308 | CG2 | ILE | A | 576 | −4.371 | 14.222 | 22.053 | 1.00 | 12.83 |
| ATOM | 309 | CG1 | ILE | A | 576 | −1.933 | 14.802 | 21.965 | 1.00 | 17.73 |
| ATOM | 310 | CD1 | ILE | A | 576 | −2.154 | 16.292 | 21.798 | 1.00 | 13.34 |
| ATOM | 311 | C | ILE | A | 576 | −3.880 | 11.636 | 20.960 | 1.00 | 12.34 |
| ATOM | 312 | O | ILE | A | 576 | −4.674 | 11.103 | 21.737 | 1.00 | 13.64 |
| ATOM | 313 | N | ARG | A | 577 | −3.985 | 11.532 | 19.638 | 1.00 | 13.14 |
| ATOM | 314 | CA | ARG | A | 577 | −5.071 | 10.772 | 19.032 | 1.00 | 13.22 |
| ATOM | 315 | CB | ARG | A | 577 | −5.029 | 10.888 | 17.503 | 1.00 | 12.21 |
| ATOM | 316 | CG | ARG | A | 577 | −6.113 | 10.084 | 16.795 | 1.00 | 12.42 |
| ATOM | 317 | CD | ARG | A | 577 | −7.492 | 10.383 | 17.365 | 1.00 | 10.79 |
| ATOM | 318 | NE | ARG | A | 577 | −7.912 | 11.757 | 17.111 | 1.00 | 18.46 |
| ATOM | 319 | CZ | ARG | A | 577 | −9.015 | 12.304 | 17.612 | 1.00 | 16.47 |
| ATOM | 320 | NH1 | ARG | A | 577 | −9.319 | 13.564 | 17.325 | 1.00 | 24.31 |
| ATOM | 321 | NH2 | ARG | A | 577 | −9.809 | 11.596 | 18.405 | 1.00 | 18.21 |
| ATOM | 322 | C | ARG | A | 577 | −5.029 | 9.304 | 19.455 | 1.00 | 12.45 |
| ATOM | 323 | O | ARG | A | 577 | −6.069 | 8.675 | 19.625 | 1.00 | 11.98 |
| ATOM | 324 | N | MET | A | 578 | −3.831 | 8.756 | 19.630 | 1.00 | 10.43 |
| ATOM | 325 | CA | MET | A | 578 | −3.716 | 7.367 | 20.058 | 1.00 | 11.28 |
| ATOM | 326 | CB | MET | A | 578 | −2.245 | 6.934 | 20.090 | 1.00 | 11.91 |
| ATOM | 327 | CG | MET | A | 578 | −1.605 | 6.808 | 18.716 | 1.00 | 12.93 |
| ATOM | 328 | SD | MET | A | 578 | 0.134 | 6.324 | 18.830 | 1.00 | 16.07 |
| ATOM | 329 | CE | MET | A | 578 | 0.781 | 7.021 | 17.300 | 1.00 | 19.76 |
| ATOM | 330 | C | MET | A | 578 | −4.332 | 7.191 | 21.445 | 1.00 | 12.18 |
| ATOM | 331 | O | MET | A | 578 | −5.103 | 6.260 | 21.675 | 1.00 | 11.26 |
| ATOM | 332 | N | PHE | A | 579 | −3.982 | 8.080 | 22.372 | 1.00 | 11.60 |
| ATOM | 333 | CA | PHE | A | 579 | −4.521 | 8.004 | 23.725 | 1.00 | 11.60 |
| ATOM | 334 | CB | PHE | A | 579 | −3.921 | 9.097 | 24.619 | 1.00 | 12.17 |
| ATOM | 335 | CG | PHE | A | 579 | −2.584 | 8.748 | 25.201 | 1.00 | 9.68 |
| ATOM | 336 | CD1 | PHE | A | 579 | −1.415 | 9.023 | 24.510 | 1.00 | 13.42 |
| ATOM | 337 | CD2 | PHE | A | 579 | −2.497 | 8.148 | 26.450 | 1.00 | 12.49 |
| ATOM | 338 | CE1 | PHE | A | 579 | −0.178 | 8.709 | 25.054 | 1.00 | 10.87 |
| ATOM | 339 | CE2 | PHE | A | 579 | −1.264 | 7.829 | 27.000 | 1.00 | 13.01 |
| ATOM | 340 | CZ | PHE | A | 579 | −0.103 | 8.113 | 26.301 | 1.00 | 12.83 |
| ATOM | 341 | C | PHE | A | 579 | −6.040 | 8.170 | 23.708 | 1.00 | 12.84 |
| ATOM | 342 | O | PHE | A | 579 | −6.765 | 7.429 | 24.367 | 1.00 | 12.73 |
| ATOM | 343 | N | THR | A | 580 | −6.504 | 9.151 | 22.943 | 1.00 | 15.04 |
| ATOM | 344 | CA | THR | A | 580 | −7.926 | 9.459 | 22.839 | 1.00 | 14.30 |
| ATOM | 345 | CB | THR | A | 580 | −8.145 | 10.702 | 21.967 | 1.00 | 10.78 |
| ATOM | 346 | OG1 | THR | A | 580 | −7.344 | 11.780 | 22.469 | 1.00 | 18.08 |
| ATOM | 347 | CG2 | THR | A | 580 | −9.610 | 11.115 | 21.987 | 1.00 | 13.16 |
| ATOM | 348 | C | THR | A | 580 | −8.772 | 8.327 | 22.273 | 1.00 | 14.34 |
| ATOM | 349 | O | THR | A | 580 | −9.774 | 7.936 | 22.871 | 1.00 | 14.67 |
| ATOM | 350 | N | ASP | A | 581 | −8.367 | 7.806 | 21.119 | 1.00 | 15.75 |
| ATOM | 351 | CA | ASP | A | 581 | −9.106 | 6.731 | 20.466 | 1.00 | 14.61 |
| ATOM | 352 | CB | ASP | A | 581 | −8.724 | 6.668 | 18.990 | 1.00 | 16.27 |
| ATOM | 353 | CG | ASP | A | 581 | −9.398 | 7.757 | 18.181 | 1.00 | 20.38 |
| ATOM | 354 | OD1 | ASP | A | 581 | −9.762 | 8.797 | 18.776 | 1.00 | 15.70 |
| ATOM | 355 | OD2 | ASP | A | 581 | −9.560 | 7.580 | 16.958 | 1.00 | 17.57 |
| ATOM | 356 | C | ASP | A | 581 | −8.970 | 5.365 | 21.123 | 1.00 | 15.19 |
| ATOM | 357 | O | ASP | A | 581 | −9.627 | 4.402 | 20.719 | 1.00 | 14.62 |
| ATOM | 358 | N | LEU | A | 582 | −8.110 | 5.275 | 22.130 | 1.00 | 13.55 |
| ATOM | 359 | CA | LEU | A | 582 | −7.957 | 4.032 | 22.867 | 1.00 | 14.36 |
| ATOM | 360 | CB | LEU | A | 582 | −6.484 | 3.739 | 23.156 | 1.00 | 16.24 |
| ATOM | 361 | CG | LEU | A | 582 | −5.770 | 3.024 | 22.006 | 1.00 | 14.56 |
| ATOM | 362 | CD1 | LEU | A | 582 | −4.272 | 2.968 | 22.250 | 1.00 | 12.82 |
| ATOM | 363 | CD2 | LEU | A | 582 | −6.351 | 1.625 | 21.865 | 1.00 | 12.05 |
| ATOM | 364 | C | LEU | A | 582 | −8.739 | 4.189 | 24.168 | 1.00 | 17.31 |
| ATOM | 365 | O | LEU | A | 582 | −8.617 | 3.378 | 25.081 | 1.00 | 17.82 |
| ATOM | 366 | N | ASN | A | 583 | −9.533 | 5.255 | 24.246 | 1.00 | 17.93 |
| ATOM | 367 | CA | ASN | A | 583 | −10.361 | 5.500 | 25.420 | 1.00 | 21.01 |
| ATOM | 368 | CB | ASN | A | 583 | −11.405 | 4.377 | 25.525 | 1.00 | 22.07 |
| ATOM | 369 | CG | ASN | A | 583 | −12.368 | 4.564 | 26.682 | 1.00 | 34.95 |
| ATOM | 370 | OD1 | ASN | A | 583 | −12.921 | 5.645 | 26.879 | 1.00 | 34.93 |
| ATOM | 371 | ND2 | ASN | A | 583 | −12.589 | 3.496 | 27.444 | 1.00 | 39.94 |
| ATOM | 372 | C | ASN | A | 583 | −9.516 | 5.575 | 26.693 | 1.00 | 23.16 |
| ATOM | 373 | O | ASN | A | 583 | −9.866 | 4.978 | 27.713 | 1.00 | 27.41 |
| ATOM | 374 | N | LEU | A | 584 | −8.406 | 6.313 | 26.634 | 1.00 | 18.24 |
| ATOM | 375 | CA | LEU | A | 584 | −7.517 | 6.446 | 27.791 | 1.00 | 18.39 |
| ATOM | 376 | CB | LEU | A | 584 | −6.060 | 6.188 | 27.379 | 1.00 | 13.74 |
| ATOM | 377 | CG | LEU | A | 584 | −5.713 | 4.823 | 26.778 | 1.00 | 13.61 |
| ATOM | 378 | CD1 | LEU | A | 584 | −4.226 | 4.789 | 26.412 | 1.00 | 16.18 |
| ATOM | 379 | CD2 | LEU | A | 584 | −6.050 | 3.726 | 27.769 | 1.00 | 15.78 |
| ATOM | 380 | C | LEU | A | 584 | −7.591 | 7.807 | 28.486 | 1.00 | 18.85 |
| ATOM | 381 | O | LEU | A | 584 | −7.377 | 7.904 | 29.695 | 1.00 | 18.54 |
| ATOM | 382 | N | VAL | A | 585 | −7.890 | 8.853 | 27.721 | 1.00 | 18.53 |
| ATOM | 383 | CA | VAL | A | 585 | −7.956 | 10.211 | 28.258 | 1.00 | 20.90 |
| ATOM | 384 | CB | VAL | A | 585 | −8.157 | 11.234 | 27.123 | 1.00 | 25.63 |
| ATOM | 385 | CG1 | VAL | A | 585 | −8.083 | 12.648 | 27.672 | 1.00 | 26.57 |
| ATOM | 386 | CG2 | VAL | A | 585 | −7.102 | 11.024 | 26.054 | 1.00 | 26.78 |
| ATOM | 387 | C | VAL | A | 585 | −9.049 | 10.416 | 29.307 | 1.00 | 21.72 |
| ATOM | 388 | O | VAL | A | 585 | −8.771 | 10.839 | 30.429 | 1.00 | 21.94 |
| ATOM | 389 | N | GLN | A | 586 | −10.291 | 10.118 | 28.945 | 1.00 | 22.79 |
| ATOM | 390 | CA | GLN | A | 586 | −11.394 | 10.290 | 29.881 | 1.00 | 25.11 |
| ATOM | 391 | CB | GLN | A | 586 | −12.728 | 10.306 | 29.131 | 1.00 | 34.29 |
| ATOM | 392 | CG | GLN | A | 586 | −13.719 | 11.317 | 29.678 | 1.00 | 39.65 |
| ATOM | 393 | CD | GLN | A | 586 | −13.155 | 12.725 | 29.663 | 1.00 | 50.17 |
| ATOM | 394 | OE1 | GLN | A | 586 | −12.715 | 13.218 | 28.622 | 1.00 | 52.20 |
| ATOM | 395 | NE2 | GLN | A | 586 | −13.160 | 13.380 | 30.820 | 1.00 | 48.68 |
| ATOM | 396 | C | GLN | A | 586 | −11.402 | 9.172 | 30.914 | 1.00 | 22.36 |
| ATOM | 397 | O | GLN | A | 586 | −11.587 | 9.413 | 32.108 | 1.00 | 23.81 |
| ATOM | 398 | N | ASN | A | 587 | −11.195 | 7.948 | 30.441 | 1.00 | 22.47 |
| ATOM | 399 | CA | ASN | A | 587 | −11.181 | 6.769 | 31.298 | 1.00 | 19.07 |
| ATOM | 400 | CB | ASN | A | 587 | −10.651 | 5.565 | 30.507 | 1.00 | 21.06 |
| ATOM | 401 | CG | ASN | A | 587 | −10.944 | 4.236 | 31.179 | 1.00 | 29.14 |
| ATOM | 402 | OD1 | ASN | A | 587 | −11.098 | 4.157 | 32.397 | 1.00 | 21.26 |
| ATOM | 403 | ND2 | ASN | A | 587 | −11.002 | 3.174 | 30.382 | 1.00 | 33.44 |
| ATOM | 404 | C | ASN | A | 587 | −10.319 | 6.981 | 32.542 | 1.00 | 20.86 |
| ATOM | 405 | O | ASN | A | 587 | −10.727 | 6.646 | 33.656 | 1.00 | 19.43 |
| ATOM | 406 | N | PHE | A | 588 | −9.134 | 7.553 | 32.357 | 1.00 | 18.57 |
| ATOM | 407 | CA | PHE | A | 588 | −8.227 | 7.753 | 33.480 | 1.00 | 18.65 |
| ATOM | 408 | CB | PHE | A | 588 | −6.880 | 7.109 | 33.145 | 1.00 | 13.88 |
| ATOM | 409 | CG | PHE | A | 588 | −6.976 | 5.624 | 32.948 | 1.00 | 14.75 |
| ATOM | 410 | CD1 | PHE | A | 588 | −7.272 | 4.791 | 34.022 | 1.00 | 13.58 |
| ATOM | 411 | CD2 | PHE | A | 588 | −6.860 | 5.065 | 31.686 | 1.00 | 12.05 |
| ATOM | 412 | CE1 | PHE | A | 588 | −7.456 | 3.431 | 33.837 | 1.00 | 13.16 |
| ATOM | 413 | CE2 | PHE | A | 588 | −7.044 | 3.704 | 31.496 | 1.00 | 10.45 |
| ATOM | 414 | CZ | PHE | A | 588 | −7.344 | 2.888 | 32.576 | 1.00 | 16.21 |
| ATOM | 415 | C | PHE | A | 588 | −8.063 | 9.190 | 33.966 | 1.00 | 20.57 |
| ATOM | 416 | O | PHE | A | 588 | −7.072 | 9.538 | 34.611 | 1.00 | 18.90 |
| ATOM | 417 | N | GLN | A | 589 | −9.052 | 10.020 | 33.662 | 1.00 | 21.41 |
| ATOM | 418 | CA | GLN | A | 589 | −9.057 | 11.410 | 34.110 | 1.00 | 23.36 |
| ATOM | 419 | CB | GLN | A | 589 | −9.213 | 11.449 | 35.634 | 1.00 | 28.73 |
| ATOM | 420 | CG | GLN | A | 589 | −10.311 | 10.551 | 36.194 | 1.00 | 40.43 |
| ATOM | 421 | CD | GLN | A | 589 | −11.701 | 11.003 | 35.797 | 1.00 | 42.17 |
| ATOM | 422 | OE1 | GLN | A | 589 | −12.058 | 12.167 | 35.971 | 1.00 | 38.12 |
| ATOM | 423 | NE2 | GLN | A | 589 | −12.498 | 10.080 | 35.270 | 1.00 | 47.35 |
| ATOM | 424 | C | GLN | A | 589 | −7.805 | 12.201 | 33.725 | 1.00 | 20.90 |
| ATOM | 425 | O | GLN | A | 589 | −7.282 | 12.972 | 34.531 | 1.00 | 21.16 |
| ATOM | 426 | N | MET | A | 590 | −7.321 | 12.019 | 32.502 | 1.00 | 22.12 |
| ATOM | 427 | CA | MET | A | 590 | −6.133 | 12.745 | 32.069 | 1.00 | 21.73 |
| ATOM | 428 | CB | MET | A | 590 | −5.463 | 12.035 | 30.893 | 1.00 | 22.93 |
| ATOM | 429 | CG | MET | A | 590 | −4.751 | 10.752 | 31.257 | 1.00 | 24.06 |
| ATOM | 430 | SD | MET | A | 590 | −3.865 | 10.087 | 29.838 | 1.00 | 19.72 |
| ATOM | 431 | CE | MET | A | 590 | −3.631 | 8.404 | 30.357 | 1.00 | 17.35 |
| ATOM | 432 | C | MET | A | 590 | −6.455 | 14.172 | 31.654 | 1.00 | 22.68 |
| ATOM | 433 | O | MET | A | 590 | −7.295 | 14.398 | 30.784 | 1.00 | 23.69 |
| ATOM | 434 | N | LYS | A | 591 | −5.792 | 15.138 | 32.278 | 1.00 | 20.35 |
| ATOM | 435 | CA | LYS | A | 591 | −6.013 | 16.530 | 31.919 | 1.00 | 20.85 |
| ATOM | 436 | CB | LYS | A | 591 | −5.554 | 17.460 | 33.045 | 1.00 | 22.95 |
| ATOM | 437 | CG | LYS | A | 591 | −6.472 | 17.422 | 34.262 | 1.00 | 28.61 |
| ATOM | 438 | CD | LYS | A | 591 | −6.065 | 18.438 | 35.315 | 1.00 | 37.59 |
| ATOM | 439 | CE | LYS | A | 591 | −7.106 | 18.527 | 36.423 | 1.00 | 42.92 |
| ATOM | 440 | NZ | LYS | A | 591 | −8.417 | 19.026 | 35.915 | 1.00 | 51.43 |
| ATOM | 441 | C | LYS | A | 591 | −5.235 | 16.800 | 30.636 | 1.00 | 20.72 |
| ATOM | 442 | O | LYS | A | 591 | −4.048 | 16.486 | 30.537 | 1.00 | 21.29 |
| ATOM | 443 | N | HIS | A | 592 | −5.920 | 17.375 | 29.654 | 1.00 | 20.84 |
| ATOM | 444 | CA | HIS | A | 592 | −5.333 | 17.670 | 28.351 | 1.00 | 21.11 |
| ATOM | 445 | CB | HIS | A | 592 | −6.273 | 18.577 | 27.553 | 1.00 | 21.39 |
| ATOM | 446 | CG | HIS | A | 592 | −5.884 | 18.737 | 26.125 | 1.00 | 18.05 |
| ATOM | 447 | CD2 | HIS | A | 592 | −5.427 | 19.798 | 25.424 | 1.00 | 24.75 |
| ATOM | 448 | ND1 | HIS | A | 592 | −5.926 | 17.685 | 25.213 | 1.00 | 22.74 |
| ATOM | 449 | CE1 | HIS | A | 592 | −5.518 | 18.102 | 24.044 | 1.00 | 29.20 |
| ATOM | 450 | NE2 | HIS | A | 592 | −5.204 | 19.393 | 24.135 | 1.00 | 27.94 |
| ATOM | 451 | C | HIS | A | 592 | −3.937 | 18.295 | 28.377 | 1.00 | 20.49 |
| ATOM | 452 | O | HIS | A | 592 | −3.034 | 17.848 | 27.661 | 1.00 | 19.49 |
| ATOM | 453 | N | GLU | A | 593 | −3.765 | 19.338 | 29.184 | 1.00 | 19.12 |
| ATOM | 454 | CA | GLU | A | 593 | −2.481 | 20.019 | 29.279 | 1.00 | 18.26 |
| ATOM | 455 | CB | GLU | A | 593 | −2.592 | 21.243 | 30.192 | 1.00 | 23.02 |
| ATOM | 456 | CG | GLU | A | 593 | −1.267 | 21.957 | 30.416 | 1.00 | 32.52 |
| ATOM | 457 | CD | GLU | A | 593 | −1.422 | 23.296 | 31.116 | 1.00 | 44.01 |
| ATOM | 458 | OE1 | GLU | A | 593 | −2.062 | 24.200 | 30.535 | 1.00 | 41.08 |
| ATOM | 459 | OE2 | GLU | A | 593 | −0.901 | 23.446 | 32.244 | 1.00 | 45.85 |
| ATOM | 460 | C | GLU | A | 593 | −1.381 | 19.099 | 29.793 | 1.00 | 18.42 |
| ATOM | 461 | O | GLU | A | 593 | −0.230 | 19.195 | 29.365 | 1.00 | 16.87 |
| ATOM | 462 | N | VAL | A | 594 | −1.738 | 18.209 | 30.710 | 1.00 | 18.22 |
| ATOM | 463 | CA | VAL | A | 594 | −0.770 | 17.275 | 31.277 | 1.00 | 17.13 |
| ATOM | 464 | CB | VAL | A | 594 | −1.372 | 16.506 | 32.465 | 1.00 | 16.16 |
| ATOM | 465 | CG1 | VAL | A | 594 | −0.314 | 15.605 | 33.085 | 1.00 | 20.37 |
| ATOM | 466 | CG2 | VAL | A | 594 | −1.911 | 17.481 | 33.491 | 1.00 | 22.54 |
| ATOM | 467 | C | VAL | A | 594 | −0.299 | 16.263 | 30.235 | 1.00 | 15.25 |
| ATOM | 468 | O | VAL | A | 594 | 0.902 | 16.005 | 30.101 | 1.00 | 16.26 |
| ATOM | 469 | N | LEU | A | 595 | −1.246 | 15.681 | 29.504 | 1.00 | 13.83 |
| ATOM | 470 | CA | LEU | A | 595 | −0.897 | 14.705 | 28.478 | 1.00 | 14.01 |
| ATOM | 471 | CB | LEU | A | 595 | −2.163 | 14.147 | 27.818 | 1.00 | 9.50 |
| ATOM | 472 | CG | LEU | A | 595 | −1.969 | 13.167 | 26.653 | 1.00 | 18.45 |
| ATOM | 473 | CD1 | LEU | A | 595 | −1.068 | 12.014 | 27.076 | 1.00 | 12.38 |
| ATOM | 474 | CD2 | LEU | A | 595 | −3.318 | 12.643 | 26.201 | 1.00 | 14.12 |
| ATOM | 475 | C | LEU | A | 595 | −0.002 | 15.372 | 27.438 | 1.00 | 13.61 |
| ATOM | 476 | O | LEU | A | 595 | 1.004 | 14.804 | 27.010 | 1.00 | 13.98 |
| ATOM | 477 | N | CYS | A | 596 | −0.358 | 16.588 | 27.037 | 1.00 | 14.70 |
| ATOM | 478 | CA | CYS | A | 596 | 0.448 | 17.303 | 26.058 | 1.00 | 12.24 |
| ATOM | 479 | CB | CYS | A | 596 | −0.191 | 18.649 | 25.709 | 1.00 | 15.21 |
| ATOM | 480 | SG | CYS | A | 596 | −1.702 | 18.507 | 24.749 | 1.00 | 17.45 |
| ATOM | 481 | C | CYS | A | 596 | 1.861 | 17.534 | 26.586 | 1.00 | 12.70 |
| ATOM | 482 | O | CYS | A | 596 | 2.839 | 17.327 | 25.868 | 1.00 | 11.06 |
| ATOM | 483 | N | ARG | A | 597 | 1.970 | 17.959 | 27.841 | 1.00 | 11.36 |
| ATOM | 484 | CA | ARG | A | 597 | 3.283 | 18.211 | 28.420 | 1.00 | 11.51 |
| ATOM | 485 | CB | ARG | A | 597 | 3.141 | 18.836 | 29.815 | 1.00 | 14.74 |
| ATOM | 486 | CG | ARG | A | 597 | 4.463 | 19.343 | 30.388 | 1.00 | 15.86 |
| ATOM | 487 | CD | ARG | A | 597 | 4.294 | 20.067 | 31.727 | 1.00 | 20.22 |
| ATOM | 488 | NE | ARG | A | 597 | 3.565 | 21.332 | 31.614 | 1.00 | 21.93 |
| ATOM | 489 | CZ | ARG | A | 597 | 2.279 | 21.493 | 31.916 | 1.00 | 27.00 |
| ATOM | 490 | NH1 | ARG | A | 597 | 1.560 | 20.469 | 32.357 | 1.00 | 22.77 |
| ATOM | 491 | NH2 | ARG | A | 597 | 1.708 | 22.683 | 31.782 | 1.00 | 30.27 |
| ATOM | 492 | C | ARG | A | 597 | 4.100 | 16.919 | 28.494 | 1.00 | 11.80 |
| ATOM | 493 | O | ARG | A | 597 | 5.287 | 16.909 | 28.162 | 1.00 | 10.48 |
| ATOM | 494 | N | TRP | A | 598 | 3.454 | 15.830 | 28.905 | 1.00 | 11.97 |
| ATOM | 495 | CA | TRP | A | 598 | 4.112 | 14.530 | 29.022 | 1.00 | 9.62 |
| ATOM | 496 | CB | TRP | A | 598 | 3.125 | 13.488 | 29.556 | 1.00 | 11.57 |
| ATOM | 497 | CG | TRP | A | 598 | 3.717 | 12.110 | 29.702 | 1.00 | 10.65 |
| ATOM | 498 | CD2 | TRP | A | 598 | 3.609 | 11.028 | 28.771 | 1.00 | 9.66 |
| ATOM | 499 | CE2 | TRP | A | 598 | 4.350 | 9.948 | 29.298 | 1.00 | 8.24 |
| ATOM | 500 | CE3 | TRP | A | 598 | 2.964 | 10.866 | 27.541 | 1.00 | 11.87 |
| ATOM | 501 | CD1 | TRP | A | 598 | 4.494 | 11.656 | 30.728 | 1.00 | 9.74 |
| ATOM | 502 | NE1 | TRP | A | 598 | 4.878 | 10.359 | 30.492 | 1.00 | 11.42 |
| ATOM | 503 | CZ2 | TRP | A | 598 | 4.459 | 8.720 | 28.635 | 1.00 | 7.38 |
| ATOM | 504 | CZ3 | TRP | A | 598 | 3.075 | 9.646 | 26.886 | 1.00 | 9.43 |
| ATOM | 505 | CH2 | TRP | A | 598 | 3.818 | 8.593 | 27.434 | 1.00 | 10.02 |
| ATOM | 506 | C | TRP | A | 598 | 4.656 | 14.064 | 27.670 | 1.00 | 12.66 |
| ATOM | 507 | O | TRP | A | 598 | 5.804 | 13.617 | 27.568 | 1.00 | 11.33 |
| ATOM | 508 | N | ILE | A | 599 | 3.823 | 14.160 | 26.637 | 1.00 | 9.84 |
| ATOM | 509 | CA | ILE | A | 599 | 4.232 | 13.748 | 25.298 | 1.00 | 12.03 |
| ATOM | 510 | CB | ILE | A | 599 | 3.101 | 13.974 | 24.270 | 1.00 | 11.30 |
| ATOM | 511 | CG2 | ILE | A | 599 | 3.648 | 13.807 | 22.856 | 1.00 | 13.41 |
| ATOM | 512 | CG1 | ILE | A | 599 | 1.953 | 13.000 | 24.546 | 1.00 | 13.16 |
| ATOM | 513 | CD1 | ILE | A | 599 | 0.732 | 13.218 | 23.668 | 1.00 | 13.06 |
| ATOM | 514 | C | ILE | A | 599 | 5.460 | 14.533 | 24.855 | 1.00 | 14.21 |
| ATOM | 515 | O | ILE | A | 599 | 6.414 | 13.969 | 24.308 | 1.00 | 11.47 |
| ATOM | 516 | N | LEU | A | 600 | 5.439 | 15.839 | 25.094 | 1.00 | 14.33 |
| ATOM | 517 | CA | LEU | A | 600 | 6.566 | 16.670 | 24.707 | 1.00 | 13.61 |
| ATOM | 518 | CB | LEU | A | 600 | 6.214 | 18.151 | 24.882 | 1.00 | 13.53 |
| ATOM | 519 | CG | LEU | A | 600 | 5.073 | 18.621 | 23.971 | 1.00 | 12.80 |
| ATOM | 520 | CD1 | LEU | A | 600 | 4.652 | 20.036 | 24.336 | 1.00 | 15.66 |
| ATOM | 521 | CD2 | LEU | A | 600 | 5.518 | 18.557 | 22.520 | 1.00 | 13.09 |
| ATOM | 522 | C | LEU | A | 600 | 7.800 | 16.297 | 25.523 | 1.00 | 10.78 |
| ATOM | 523 | O | LEU | A | 600 | 8.917 | 16.324 | 25.011 | 1.00 | 10.47 |
| ATOM | 524 | N | SER | A | 601 | 7.601 | 15.925 | 26.788 | 1.00 | 10.50 |
| ATOM | 525 | CA | SER | A | 601 | 8.734 | 15.540 | 27.624 | 1.00 | 8.54 |
| ATOM | 526 | CB | SER | A | 601 | 8.303 | 15.398 | 29.085 | 1.00 | 9.00 |
| ATOM | 527 | OG | SER | A | 601 | 8.036 | 16.678 | 29.634 | 1.00 | 9.87 |
| ATOM | 528 | C | SER | A | 601 | 9.342 | 14.239 | 27.129 | 1.00 | 9.99 |
| ATOM | 529 | O | SER | A | 601 | 10.563 | 14.082 | 27.104 | 1.00 | 10.43 |
| ATOM | 530 | N | VAL | A | 602 | 8.487 | 13.300 | 26.739 | 1.00 | 10.77 |
| ATOM | 531 | CA | VAL | A | 602 | 8.968 | 12.024 | 26.223 | 1.00 | 10.98 |
| ATOM | 532 | CB | VAL | A | 602 | 7.791 | 11.081 | 25.874 | 1.00 | 12.52 |
| ATOM | 533 | CG1 | VAL | A | 602 | 8.303 | 9.856 | 25.138 | 1.00 | 9.22 |
| ATOM | 534 | CG2 | VAL | A | 602 | 7.068 | 10.658 | 27.152 | 1.00 | 11.71 |
| ATOM | 535 | C | VAL | A | 602 | 9.794 | 12.290 | 24.967 | 1.00 | 12.57 |
| ATOM | 536 | O | VAL | A | 602 | 10.935 | 11.848 | 24.855 | 1.00 | 11.99 |
| ATOM | 537 | N | LYS | A | 603 | 9.220 | 13.043 | 24.034 | 1.00 | 12.97 |
| ATOM | 538 | CA | LYS | A | 603 | 9.906 | 13.363 | 22.792 | 1.00 | 11.73 |
| ATOM | 539 | CB | LYS | A | 603 | 9.025 | 14.268 | 21.928 | 1.00 | 14.00 |
| ATOM | 540 | CG | LYS | A | 603 | 9.666 | 14.688 | 20.620 | 1.00 | 14.56 |
| ATOM | 541 | CD | LYS | A | 603 | 8.656 | 15.317 | 19.686 | 1.00 | 16.82 |
| ATOM | 542 | CE | LYS | A | 603 | 8.088 | 16.603 | 20.262 | 1.00 | 24.97 |
| ATOM | 543 | NZ | LYS | A | 603 | 7.169 | 17.256 | 19.300 | 1.00 | 39.23 |
| ATOM | 544 | C | LYS | A | 603 | 11.256 | 14.035 | 23.042 | 1.00 | 11.50 |
| ATOM | 545 | O | LYS | A | 603 | 12.252 | 13.694 | 22.414 | 1.00 | 14.04 |
| ATOM | 546 | N | LYS | A | 604 | 11.279 | 14.985 | 23.968 | 1.00 | 11.12 |
| ATOM | 547 | CA | LYS | A | 604 | 12.501 | 15.714 | 24.293 | 1.00 | 13.84 |
| ATOM | 548 | CB | LYS | A | 604 | 12.181 | 16.849 | 25.274 | 1.00 | 16.59 |
| ATOM | 549 | CG | LYS | A | 604 | 13.403 | 17.589 | 25.788 | 1.00 | 18.98 |
| ATOM | 550 | CD | LYS | A | 604 | 13.016 | 18.673 | 26.784 | 1.00 | 25.53 |
| ATOM | 551 | CE | LYS | A | 604 | 14.251 | 19.335 | 27.391 | 1.00 | 31.51 |
| ATOM | 552 | NZ | LYS | A | 604 | 13.893 | 20.351 | 28.424 | 1.00 | 29.80 |
| ATOM | 553 | C | LYS | A | 604 | 13.582 | 14.824 | 24.890 | 1.00 | 12.98 |
| ATOM | 554 | O | LYS | A | 604 | 14.776 | 15.073 | 24.710 | 1.00 | 13.10 |
| ATOM | 555 | N | ASN | A | 605 | 13.173 | 13.773 | 25.587 | 1.00 | 12.62 |
| ATOM | 556 | CA | ASN | A | 605 | 14.146 | 12.901 | 26.220 | 1.00 | 13.04 |
| ATOM | 557 | CB | ASN | A | 605 | 13.555 | 12.336 | 27.507 | 1.00 | 13.13 |
| ATOM | 558 | CG | ASN | A | 605 | 13.579 | 13.356 | 28.638 | 1.00 | 32.58 |
| ATOM | 559 | OD1 | ASN | A | 605 | 14.638 | 13.650 | 29.197 | 1.00 | 25.76 |
| ATOM | 560 | ND2 | ASN | A | 605 | 12.418 | 13.918 | 28.962 | 1.00 | 30.95 |
| ATOM | 561 | C | ASN | A | 605 | 14.757 | 11.809 | 25.352 | 1.00 | 15.12 |
| ATOM | 562 | O | ASN | A | 605 | 15.342 | 10.848 | 25.858 | 1.00 | 18.48 |
| ATOM | 563 | N | TYR | A | 606 | 14.612 | 11.961 | 24.042 | 1.00 | 10.60 |
| ATOM | 564 | CA | TYR | A | 606 | 15.233 | 11.051 | 23.093 | 1.00 | 10.45 |
| ATOM | 565 | CB | TYR | A | 606 | 14.254 | 10.645 | 21.982 | 1.00 | 10.34 |
| ATOM | 566 | CG | TYR | A | 606 | 13.420 | 9.444 | 22.358 | 1.00 | 11.02 |
| ATOM | 567 | CD1 | TYR | A | 606 | 13.950 | 8.156 | 22.297 | 1.00 | 8.31 |
| ATOM | 568 | CE1 | TYR | A | 606 | 13.222 | 7.061 | 22.752 | 1.00 | 8.28 |
| ATOM | 569 | CD2 | TYR | A | 606 | 12.139 | 9.601 | 22.874 | 1.00 | 12.47 |
| ATOM | 570 | CE2 | TYR | A | 606 | 11.410 | 8.516 | 23.335 | 1.00 | 15.05 |
| ATOM | 571 | CZ | TYR | A | 606 | 11.955 | 7.252 | 23.275 | 1.00 | 15.46 |
| ATOM | 572 | OH | TYR | A | 606 | 11.227 | 6.188 | 23.773 | 1.00 | 12.36 |
| ATOM | 573 | C | TYR | A | 606 | 16.362 | 11.908 | 22.539 | 1.00 | 11.41 |
| ATOM | 574 | O | TYR | A | 606 | 16.216 | 13.129 | 22.428 | 1.00 | 14.91 |
| ATOM | 575 | N | ARG | A | 607 | 17.499 | 11.293 | 22.234 | 1.00 | 11.41 |
| ATOM | 576 | CA | ARG | A | 607 | 18.627 | 12.042 | 21.700 | 1.00 | 10.90 |
| ATOM | 577 | CB | ARG | A | 607 | 19.933 | 11.322 | 22.034 | 1.00 | 12.68 |
| ATOM | 578 | CG | ARG | A | 607 | 20.078 | 11.019 | 23.520 | 1.00 | 10.36 |
| ATOM | 579 | CD | ARG | A | 607 | 21.460 | 10.489 | 23.870 | 1.00 | 13.25 |
| ATOM | 580 | NE | ARG | A | 607 | 21.492 | 9.929 | 25.221 | 1.00 | 9.55 |
| ATOM | 581 | CZ | ARG | A | 607 | 22.601 | 9.556 | 25.854 | 1.00 | 18.33 |
| ATOM | 582 | NH1 | ARG | A | 607 | 23.781 | 9.686 | 25.262 | 1.00 | 13.50 |
| ATOM | 583 | NH2 | ARG | A | 607 | 22.530 | 9.045 | 27.077 | 1.00 | 17.68 |
| ATOM | 584 | C | ARG | A | 607 | 18.425 | 12.150 | 20.194 | 1.00 | 10.45 |
| ATOM | 585 | O | ARG | A | 607 | 18.616 | 11.182 | 19.458 | 1.00 | 12.53 |
| ATOM | 586 | N | LYS | A | 608 | 18.052 | 13.340 | 19.738 | 1.00 | 11.88 |
| ATOM | 587 | CA | LYS | A | 608 | 17.755 | 13.555 | 18.323 | 1.00 | 13.33 |
| ATOM | 588 | CB | LYS | A | 608 | 17.273 | 14.993 | 18.104 | 1.00 | 18.33 |
| ATOM | 589 | CG | LYS | A | 608 | 18.226 | 16.064 | 18.574 | 1.00 | 34.41 |
| ATOM | 590 | CD | LYS | A | 608 | 17.558 | 17.429 | 18.538 | 1.00 | 39.16 |
| ATOM | 591 | CE | LYS | A | 608 | 18.430 | 18.485 | 19.193 | 1.00 | 44.56 |
| ATOM | 592 | NZ | LYS | A | 608 | 19.755 | 18.595 | 18.525 | 1.00 | 49.34 |
| ATOM | 593 | C | LYS | A | 608 | 18.837 | 13.220 | 17.311 | 1.00 | 13.41 |
| ATOM | 594 | O | LYS | A | 608 | 18.527 | 12.904 | 16.164 | 1.00 | 14.71 |
| ATOM | 595 | N | ASN | A | 609 | 20.098 | 13.272 | 17.724 | 1.00 | 12.65 |
| ATOM | 596 | CA | ASN | A | 609 | 21.185 | 12.977 | 16.804 | 1.00 | 15.37 |
| ATOM | 597 | CB | ASN | A | 609 | 22.383 | 13.872 | 17.121 | 1.00 | 19.55 |
| ATOM | 598 | CG | ASN | A | 609 | 22.059 | 15.347 | 16.952 | 1.00 | 21.29 |
| ATOM | 599 | OD1 | ASN | A | 609 | 21.630 | 15.779 | 15.882 | 1.00 | 28.72 |
| ATOM | 600 | ND2 | ASN | A | 609 | 22.261 | 16.126 | 18.009 | 1.00 | 36.76 |
| ATOM | 601 | C | ASN | A | 609 | 21.593 | 11.507 | 16.743 | 1.00 | 14.47 |
| ATOM | 602 | O | ASN | A | 609 | 22.486 | 11.135 | 15.981 | 1.00 | 15.29 |
| ATOM | 603 | N | VAL | A | 610 | 20.955 | 10.670 | 17.557 | 1.00 | 11.89 |
| ATOM | 604 | CA | VAL | A | 610 | 21.221 | 9.233 | 17.511 | 1.00 | 11.43 |
| ATOM | 605 | CB | VAL | A | 610 | 20.696 | 8.531 | 18.777 | 1.00 | 11.38 |
| ATOM | 606 | CG1 | VAL | A | 610 | 20.649 | 7.019 | 18.571 | 1.00 | 10.56 |
| ATOM | 607 | CG2 | VAL | A | 610 | 21.608 | 8.882 | 19.955 | 1.00 | 10.47 |
| ATOM | 608 | C | VAL | A | 610 | 20.432 | 8.809 | 16.272 | 1.00 | 12.97 |
| ATOM | 609 | O | VAL | A | 610 | 19.221 | 9.025 | 16.198 | 1.00 | 11.59 |
| ATOM | 610 | N | ALA | A | 611 | 21.131 | 8.223 | 15.302 | 1.00 | 11.37 |
| ATOM | 611 | CA | ALA | A | 611 | 20.554 | 7.833 | 14.018 | 1.00 | 12.77 |
| ATOM | 612 | CB | ALA | A | 611 | 21.579 | 7.021 | 13.223 | 1.00 | 12.83 |
| ATOM | 613 | C | ALA | A | 611 | 19.208 | 7.117 | 13.981 | 1.00 | 12.88 |
| ATOM | 614 | O | ALA | A | 611 | 18.305 | 7.522 | 13.247 | 1.00 | 14.79 |
| ATOM | 615 | N | TYR | A | 612 | 19.071 | 6.050 | 14.756 | 1.00 | 12.18 |
| ATOM | 616 | CA | TYR | A | 612 | 17.830 | 5.288 | 14.743 | 1.00 | 10.69 |
| ATOM | 617 | CB | TYR | A | 612 | 18.147 | 3.824 | 14.408 | 1.00 | 13.15 |
| ATOM | 618 | CG | TYR | A | 612 | 16.968 | 2.877 | 14.509 | 1.00 | 12.63 |
| ATOM | 619 | CD1 | TYR | A | 612 | 15.832 | 3.054 | 13.729 | 1.00 | 19.45 |
| ATOM | 620 | CE1 | TYR | A | 612 | 14.752 | 2.185 | 13.833 | 1.00 | 24.00 |
| ATOM | 621 | CD2 | TYR | A | 612 | 16.996 | 1.805 | 15.392 | 1.00 | 13.47 |
| ATOM | 622 | CE2 | TYR | A | 612 | 15.930 | 0.938 | 15.503 | 1.00 | 24.95 |
| ATOM | 623 | CZ | TYR | A | 612 | 14.812 | 1.129 | 14.722 | 1.00 | 24.27 |
| ATOM | 624 | OH | TYR | A | 612 | 13.761 | 0.245 | 14.831 | 1.00 | 25.94 |
| ATOM | 625 | C | TYR | A | 612 | 17.017 | 5.371 | 16.028 | 1.00 | 11.05 |
| ATOM | 626 | O | TYR | A | 612 | 15.812 | 5.632 | 15.989 | 1.00 | 9.25 |
| ATOM | 627 | N | HIS | A | 613 | 17.667 | 5.159 | 17.166 | 1.00 | 10.71 |
| ATOM | 628 | CA | HIS | A | 613 | 16.951 | 5.199 | 18.432 | 1.00 | 9.46 |
| ATOM | 629 | CB | HIS | A | 613 | 17.694 | 4.391 | 19.499 | 1.00 | 12.29 |
| ATOM | 630 | CG | HIS | A | 613 | 17.706 | 2.923 | 19.232 | 1.00 | 13.22 |
| ATOM | 631 | CD2 | HIS | A | 613 | 16.742 | 1.983 | 19.364 | 1.00 | 13.53 |
| ATOM | 632 | ND1 | HIS | A | 613 | 18.811 | 2.262 | 18.726 | 1.00 | 8.83 |
| ATOM | 633 | CE1 | HIS | A | 613 | 18.523 | 0.989 | 18.562 | 1.00 | 12.86 |
| ATOM | 634 | NE2 | HIS | A | 613 | 17.268 | 0.789 | 18.942 | 1.00 | 16.65 |
| ATOM | 635 | C | HIS | A | 613 | 16.686 | 6.606 | 18.936 | 1.00 | 11.81 |
| ATOM | 636 | O | HIS | A | 613 | 17.226 | 7.023 | 19.957 | 1.00 | 11.26 |
| ATOM | 637 | N | ASN | A | 614 | 15.859 | 7.333 | 18.192 | 1.00 | 12.20 |
| ATOM | 638 | CA | ASN | A | 614 | 15.459 | 8.685 | 18.556 | 1.00 | 11.65 |
| ATOM | 639 | CB | ASN | A | 614 | 15.955 | 9.702 | 17.516 | 1.00 | 19.08 |
| ATOM | 640 | CG | ASN | A | 614 | 15.631 | 9.295 | 16.090 | 1.00 | 17.63 |
| ATOM | 641 | OD1 | ASN | A | 614 | 16.529 | 9.151 | 15.253 | 1.00 | 16.40 |
| ATOM | 642 | ND2 | ASN | A | 614 | 14.349 | 9.115 | 15.802 | 1.00 | 11.39 |
| ATOM | 643 | C | ASN | A | 614 | 13.933 | 8.690 | 18.644 | 1.00 | 11.53 |
| ATOM | 644 | O | ASN | A | 614 | 13.298 | 7.647 | 18.493 | 1.00 | 9.32 |
| ATOM | 645 | N | TRP | A | 615 | 13.342 | 9.850 | 18.890 | 1.00 | 8.95 |
| ATOM | 646 | CA | TRP | A | 615 | 11.893 | 9.929 | 19.015 | 1.00 | 9.02 |
| ATOM | 647 | CB | TRP | A | 615 | 11.457 | 11.397 | 19.127 | 1.00 | 9.00 |
| ATOM | 648 | CG | TRP | A | 615 | 10.002 | 11.638 | 18.809 | 1.00 | 8.75 |
| ATOM | 649 | CD2 | TRP | A | 615 | 8.860 | 11.228 | 19.581 | 1.00 | 10.58 |
| ATOM | 650 | CE2 | TRP | A | 615 | 7.709 | 11.655 | 18.883 | 1.00 | 12.68 |
| ATOM | 651 | CE3 | TRP | A | 615 | 8.698 | 10.542 | 20.789 | 1.00 | 12.70 |
| ATOM | 652 | CD1 | TRP | A | 615 | 9.504 | 12.279 | 17.709 | 1.00 | 11.21 |
| ATOM | 653 | NE1 | TRP | A | 615 | 8.132 | 12.292 | 17.747 | 1.00 | 11.25 |
| ATOM | 654 | CZ2 | TRP | A | 615 | 6.414 | 11.418 | 19.354 | 1.00 | 12.28 |
| ATOM | 655 | CZ3 | TRP | A | 615 | 7.410 | 10.307 | 21.255 | 1.00 | 18.15 |
| ATOM | 656 | CH2 | TRP | A | 615 | 6.286 | 10.744 | 20.536 | 1.00 | 13.05 |
| ATOM | 657 | C | TRP | A | 615 | 11.090 | 9.237 | 17.910 | 1.00 | 8.08 |
| ATOM | 658 | O | TRP | A | 615 | 10.093 | 8.579 | 18.197 | 1.00 | 9.24 |
| ATOM | 659 | N | ARG | A | 616 | 11.514 | 9.365 | 16.656 | 1.00 | 9.61 |
| ATOM | 660 | CA | ARG | A | 616 | 10.750 | 8.759 | 15.570 | 1.00 | 11.24 |
| ATOM | 661 | CB | ARG | A | 616 | 11.346 | 9.129 | 14.206 | 1.00 | 9.33 |
| ATOM | 662 | CG | ARG | A | 616 | 11.241 | 10.630 | 13.885 | 1.00 | 13.33 |
| ATOM | 663 | CD | ARG | A | 616 | 9.817 | 11.169 | 14.120 | 1.00 | 11.22 |
| ATOM | 664 | NE | ARG | A | 616 | 9.705 | 12.598 | 13.823 | 1.00 | 13.61 |
| ATOM | 665 | CZ | ARG | A | 616 | 9.119 | 13.103 | 12.741 | 1.00 | 15.86 |
| ATOM | 666 | NH1 | ARG | A | 616 | 8.577 | 12.300 | 11.834 | 1.00 | 15.89 |
| ATOM | 667 | NH2 | ARG | A | 616 | 9.073 | 14.419 | 12.564 | 1.00 | 19.53 |
| ATOM | 668 | C | ARG | A | 616 | 10.607 | 7.250 | 15.705 | 1.00 | 9.88 |
| ATOM | 669 | O | ARG | A | 616 | 9.575 | 6.692 | 15.328 | 1.00 | 10.40 |
| ATOM | 670 | N | HIS | A | 617 | 11.625 | 6.582 | 16.242 | 1.00 | 7.87 |
| ATOM | 671 | CA | HIS | A | 617 | 11.514 | 5.141 | 16.419 | 1.00 | 7.37 |
| ATOM | 672 | CB | HIS | A | 617 | 12.862 | 4.523 | 16.782 | 1.00 | 11.98 |
| ATOM | 673 | CG | HIS | A | 617 | 12.742 | 3.163 | 17.390 | 1.00 | 8.70 |
| ATOM | 674 | CD2 | HIS | A | 617 | 13.043 | 2.715 | 18.632 | 1.00 | 9.51 |
| ATOM | 675 | ND1 | HIS | A | 617 | 12.206 | 2.088 | 16.713 | 1.00 | 8.99 |
| ATOM | 676 | CE1 | HIS | A | 617 | 12.183 | 1.036 | 17.510 | 1.00 | 11.83 |
| ATOM | 677 | NE2 | HIS | A | 617 | 12.684 | 1.391 | 18.681 | 1.00 | 10.58 |
| ATOM | 678 | C | HIS | A | 617 | 10.488 | 4.816 | 17.506 | 1.00 | 8.63 |
| ATOM | 679 | O | HIS | A | 617 | 9.692 | 3.888 | 17.361 | 1.00 | 10.24 |
| ATOM | 680 | N | ALA | A | 618 | 10.498 | 5.586 | 18.591 | 1.00 | 7.45 |
| ATOM | 681 | CA | ALA | A | 618 | 9.549 | 5.358 | 19.687 | 1.00 | 8.54 |
| ATOM | 682 | CB | ALA | A | 618 | 9.887 | 6.261 | 20.864 | 1.00 | 11.31 |
| ATOM | 683 | C | ALA | A | 618 | 8.126 | 5.637 | 19.210 | 1.00 | 8.39 |
| ATOM | 684 | O | ALA | A | 618 | 7.184 | 4.900 | 19.523 | 1.00 | 9.03 |
| ATOM | 685 | N | PHE | A | 619 | 7.981 | 6.725 | 18.465 | 1.00 | 8.88 |
| ATOM | 686 | CA | PHE | A | 619 | 6.695 | 7.118 | 17.911 | 1.00 | 9.63 |
| ATOM | 687 | CB | PHE | A | 619 | 6.850 | 8.418 | 17.119 | 1.00 | 12.92 |
| ATOM | 688 | CG | PHE | A | 619 | 5.618 | 8.809 | 16.357 | 1.00 | 10.44 |
| ATOM | 689 | CD1 | PHE | A | 619 | 4.432 | 9.066 | 17.021 | 1.00 | 18.21 |
| ATOM | 690 | CD2 | PHE | A | 619 | 5.646 | 8.914 | 14.974 | 1.00 | 12.41 |
| ATOM | 691 | CE1 | PHE | A | 619 | 3.291 | 9.412 | 16.324 | 1.00 | 14.74 |
| ATOM | 692 | CE2 | PHE | A | 619 | 4.508 | 9.260 | 14.270 | 1.00 | 13.16 |
| ATOM | 693 | CZ | PHE | A | 619 | 3.328 | 9.512 | 14.947 | 1.00 | 11.01 |
| ATOM | 694 | C | PHE | A | 619 | 6.150 | 6.017 | 16.996 | 1.00 | 9.52 |
| ATOM | 695 | O | PHE | A | 619 | 4.975 | 5.654 | 17.079 | 1.00 | 9.30 |
| ATOM | 696 | N | ASN | A | 620 | 7.001 | 5.490 | 16.119 | 1.00 | 10.15 |
| ATOM | 697 | CA | ASN | A | 620 | 6.583 | 4.428 | 15.204 | 1.00 | 11.34 |
| ATOM | 698 | CB | ASN | A | 620 | 7.694 | 4.123 | 14.194 | 1.00 | 9.10 |
| ATOM | 699 | CG | ASN | A | 620 | 7.658 | 5.052 | 12.999 | 1.00 | 17.16 |
| ATOM | 700 | OD1 | ASN | A | 620 | 8.665 | 5.656 | 12.634 | 1.00 | 19.51 |
| ATOM | 701 | ND2 | ASN | A | 620 | 6.490 | 5.166 | 12.382 | 1.00 | 12.88 |
| ATOM | 702 | C | ASN | A | 620 | 6.206 | 3.161 | 15.956 | 1.00 | 9.23 |
| ATOM | 703 | O | ASN | A | 620 | 5.273 | 2.457 | 15.574 | 1.00 | 10.76 |
| ATOM | 704 | N | THR | A | 621 | 6.936 | 2.871 | 17.026 | 1.00 | 8.56 |
| ATOM | 705 | CA | THR | A | 621 | 6.650 | 1.688 | 17.819 | 1.00 | 9.52 |
| ATOM | 706 | CB | THR | A | 621 | 7.707 | 1.509 | 18.928 | 1.00 | 8.96 |
| ATOM | 707 | OG1 | THR | A | 621 | 9.010 | 1.404 | 18.328 | 1.00 | 9.14 |
| ATOM | 708 | CG2 | THR | A | 621 | 7.428 | 0.244 | 19.736 | 1.00 | 7.93 |
| ATOM | 709 | C | THR | A | 621 | 5.252 | 1.821 | 18.425 | 1.00 | 10.48 |
| ATOM | 710 | O | THR | A | 621 | 4.455 | 0.879 | 18.382 | 1.00 | 10.11 |
| ATOM | 711 | N | ALA | A | 622 | 4.954 | 2.995 | 18.978 | 1.00 | 8.32 |
| ATOM | 712 | CA | ALA | A | 622 | 3.644 | 3.244 | 19.573 | 1.00 | 9.28 |
| ATOM | 713 | CB | ALA | A | 622 | 3.642 | 4.583 | 20.294 | 1.00 | 9.90 |
| ATOM | 714 | C | ALA | A | 622 | 2.567 | 3.224 | 18.488 | 1.00 | 10.02 |
| ATOM | 715 | O | ALA | A | 622 | 1.447 | 2.769 | 18.721 | 1.00 | 8.06 |
| ATOM | 716 | N | GLN | A | 623 | 2.902 | 3.708 | 17.297 | 1.00 | 7.82 |
| ATOM | 717 | CA | GLN | A | 623 | 1.927 | 3.708 | 16.211 | 1.00 | 10.17 |
| ATOM | 718 | CB | GLN | A | 623 | 2.456 | 4.487 | 15.003 | 1.00 | 8.39 |
| ATOM | 719 | CG | GLN | A | 623 | 1.473 | 4.578 | 13.825 | 1.00 | 8.23 |
| ATOM | 720 | CD | GLN | A | 623 | 1.444 | 3.319 | 12.967 | 1.00 | 13.63 |
| ATOM | 721 | OE1 | GLN | A | 623 | 2.484 | 2.730 | 12.682 | 1.00 | 12.75 |
| ATOM | 722 | NE2 | GLN | A | 623 | 0.258 | 2.919 | 12.535 | 1.00 | 11.69 |
| ATOM | 723 | C | GLN | A | 623 | 1.597 | 2.278 | 15.806 | 1.00 | 10.35 |
| ATOM | 724 | O | GLN | A | 623 | 0.449 | 1.962 | 15.501 | 1.00 | 9.61 |
| ATOM | 725 | N | CYS | A | 624 | 2.601 | 1.406 | 15.802 | 1.00 | 9.08 |
| ATOM | 726 | CA | CYS | A | 624 | 2.355 | 0.017 | 15.439 | 1.00 | 9.34 |
| ATOM | 727 | CB | CYS | A | 624 | 3.679 | −0.740 | 15.273 | 1.00 | 11.00 |
| ATOM | 728 | SG | CYS | A | 624 | 3.481 | −2.446 | 14.713 | 1.00 | 11.71 |
| ATOM | 729 | C | CYS | A | 624 | 1.495 | −0.625 | 16.529 | 1.00 | 10.17 |
| ATOM | 730 | O | CYS | A | 624 | 0.631 | −1.448 | 16.239 | 1.00 | 10.64 |
| ATOM | 731 | N | MET | A | 625 | 1.728 | −0.238 | 17.781 | 1.00 | 9.06 |
| ATOM | 732 | CA | MET | A | 625 | 0.933 | −0.764 | 18.892 | 1.00 | 9.38 |
| ATOM | 733 | CB | MET | A | 625 | 1.418 | −0.180 | 20.225 | 1.00 | 8.21 |
| ATOM | 734 | CG | MET | A | 625 | 0.692 | −0.718 | 21.466 | 1.00 | 8.16 |
| ATOM | 735 | SD | MET | A | 625 | 0.904 | −2.512 | 21.728 | 1.00 | 11.88 |
| ATOM | 736 | CE | MET | A | 625 | 2.600 | −2.586 | 22.309 | 1.00 | 11.24 |
| ATOM | 737 | C | MET | A | 625 | −0.529 | −0.376 | 18.661 | 1.00 | 9.31 |
| ATOM | 738 | O | MET | A | 625 | −1.434 | −1.208 | 18.757 | 1.00 | 10.06 |
| ATOM | 739 | N | PHE | A | 626 | −0.757 | 0.896 | 18.353 | 1.00 | 9.47 |
| ATOM | 740 | CA | PHE | A | 626 | −2.112 | 1.377 | 18.092 | 1.00 | 9.60 |
| ATOM | 741 | CB | PHE | A | 626 | −2.077 | 2.859 | 17.725 | 1.00 | 9.70 |
| ATOM | 742 | CG | PHE | A | 626 | −3.432 | 3.451 | 17.461 | 1.00 | 7.25 |
| ATOM | 743 | CD1 | PHE | A | 626 | −4.241 | 3.858 | 18.509 | 1.00 | 13.86 |
| ATOM | 744 | CD2 | PHE | A | 626 | −3.897 | 3.598 | 16.163 | 1.00 | 13.47 |
| ATOM | 745 | CE1 | PHE | A | 626 | −5.491 | 4.411 | 18.267 | 1.00 | 18.88 |
| ATOM | 746 | CE2 | PHE | A | 626 | −5.144 | 4.147 | 15.917 | 1.00 | 11.81 |
| ATOM | 747 | CZ | PHE | A | 626 | −5.942 | 4.552 | 16.970 | 1.00 | 16.53 |
| ATOM | 748 | C | PHE | A | 626 | −2.737 | 0.579 | 16.940 | 1.00 | 10.98 |
| ATOM | 749 | O | PHE | A | 626 | −3.862 | 0.084 | 17.047 | 1.00 | 12.71 |
| ATOM | 750 | N | ALA | A | 627 | −1.999 | 0.455 | 15.838 | 1.00 | 7.24 |
| ATOM | 751 | CA | ALA | A | 627 | −2.486 | −0.279 | 14.675 | 1.00 | 9.31 |
| ATOM | 752 | CB | ALA | A | 627 | −1.454 | −0.220 | 13.549 | 1.00 | 14.42 |
| ATOM | 753 | C | ALA | A | 627 | −2.803 | −1.734 | 15.024 | 1.00 | 11.79 |
| ATOM | 754 | O | ALA | A | 627 | −3.815 | −2.276 | 14.583 | 1.00 | 11.59 |
| ATOM | 755 | N | ALA | A | 628 | −1.945 | −2.361 | 15.824 | 1.00 | 10.63 |
| ATOM | 756 | CA | ALA | A | 628 | −2.158 | −3.749 | 16.219 | 1.00 | 11.00 |
| ATOM | 757 | CB | ALA | A | 628 | −0.934 | −4.280 | 16.961 | 1.00 | 11.43 |
| ATOM | 758 | C | ALA | A | 628 | −3.398 | −3.877 | 17.099 | 1.00 | 11.16 |
| ATOM | 759 | O | ALA | A | 628 | −4.152 | −4.845 | 16.994 | 1.00 | 12.30 |
| ATOM | 760 | N | LEU | A | 629 | −3.608 | −2.896 | 17.970 | 1.00 | 9.63 |
| ATOM | 761 | CA | LEU | A | 629 | −4.765 | −2.924 | 18.856 | 1.00 | 9.96 |
| ATOM | 762 | CB | LEU | A | 629 | −4.627 | −1.858 | 19.945 | 1.00 | 12.79 |
| ATOM | 763 | CG | LEU | A | 629 | −3.538 | −2.115 | 20.990 | 1.00 | 10.87 |
| ATOM | 764 | CD1 | LEU | A | 629 | −3.286 | −0.859 | 21.792 | 1.00 | 11.38 |
| ATOM | 765 | CD2 | LEU | A | 629 | −3.956 | −3.264 | 21.897 | 1.00 | 14.99 |
| ATOM | 766 | C | LEU | A | 629 | −6.051 | −2.692 | 18.073 | 1.00 | 12.31 |
| ATOM | 767 | O | LEU | A | 629 | −7.084 | −3.300 | 18.364 | 1.00 | 11.63 |
| ATOM | 768 | N | LYS | A | 630 | −5.973 | −1.822 | 17.070 | 1.00 | 11.64 |
| ATOM | 769 | CA | LYS | A | 630 | −7.125 | −1.479 | 16.242 | 1.00 | 12.87 |
| ATOM | 770 | CB | LYS | A | 630 | −6.984 | −0.037 | 15.738 | 1.00 | 15.53 |
| ATOM | 771 | CG | LYS | A | 630 | −7.084 | 1.030 | 16.820 | 1.00 | 13.30 |
| ATOM | 772 | CD | LYS | A | 630 | −8.495 | 1.104 | 17.385 | 1.00 | 19.60 |
| ATOM | 773 | CE | LYS | A | 630 | −8.698 | 2.349 | 18.245 | 1.00 | 18.93 |
| ATOM | 774 | NZ | LYS | A | 630 | −7.877 | 2.334 | 19.481 | 1.00 | 40.94 |
| ATOM | 775 | C | LYS | A | 630 | −7.323 | −2.422 | 15.049 | 1.00 | 13.54 |
| ATOM | 776 | O | LYS | A | 630 | −8.122 | −3.350 | 15.107 | 1.00 | 12.81 |
| ATOM | 777 | N | ALA | A | 631 | −6.597 | −2.170 | 13.967 | 1.00 | 13.05 |
| ATOM | 778 | CA | ALA | A | 631 | −6.701 | −2.997 | 12.770 | 1.00 | 14.94 |
| ATOM | 779 | CB | ALA | A | 631 | −5.763 | −2.460 | 11.688 | 1.00 | 17.58 |
| ATOM | 780 | C | ALA | A | 631 | −6.374 | −4.461 | 13.068 | 1.00 | 16.22 |
| ATOM | 781 | O | ALA | A | 631 | −6.960 | −5.375 | 12.479 | 1.00 | 15.44 |
| ATOM | 782 | N | GLY | A | 632 | −5.439 | −4.670 | 13.990 | 1.00 | 12.88 |
| ATOM | 783 | CA | GLY | A | 632 | −5.028 | −6.014 | 14.359 | 1.00 | 13.10 |
| ATOM | 784 | C | GLY | A | 632 | −5.995 | −6.711 | 15.298 | 1.00 | 13.12 |
| ATOM | 785 | O | GLY | A | 632 | −5.850 | −7.902 | 15.576 | 1.00 | 15.03 |
| ATOM | 786 | N | LYS | A | 633 | −6.968 | −5.959 | 15.803 | 1.00 | 13.75 |
| ATOM | 787 | CA | LYS | A | 633 | −7.997 | −6.497 | 16.692 | 1.00 | 14.20 |
| ATOM | 788 | CB | LYS | A | 633 | −8.815 | −7.566 | 15.954 | 1.00 | 13.72 |
| ATOM | 789 | CG | LYS | A | 633 | −9.438 | −7.109 | 14.631 | 1.00 | 23.64 |
| ATOM | 790 | CD | LYS | A | 633 | −10.532 | −6.072 | 14.834 | 1.00 | 30.20 |
| ATOM | 791 | CE | LYS | A | 633 | −11.182 | −5.672 | 13.510 | 1.00 | 34.87 |
| ATOM | 792 | NZ | LYS | A | 633 | −10.227 | −4.989 | 12.594 | 1.00 | 46.21 |
| ATOM | 793 | C | LYS | A | 633 | −7.464 | −7.085 | 17.997 | 1.00 | 14.18 |
| ATOM | 794 | O | LYS | A | 633 | −8.048 | −8.024 | 18.540 | 1.00 | 14.72 |
| ATOM | 795 | N | ILE | A | 634 | −6.366 | −6.536 | 18.507 | 1.00 | 13.07 |
| ATOM | 796 | CA | ILE | A | 634 | −5.801 | −7.037 | 19.755 | 1.00 | 12.44 |
| ATOM | 797 | CB | ILE | A | 634 | −4.262 | −6.819 | 19.799 | 1.00 | 13.52 |
| ATOM | 798 | CG2 | ILE | A | 634 | −3.709 | −7.236 | 21.156 | 1.00 | 14.73 |
| ATOM | 799 | CG1 | ILE | A | 634 | −3.581 | −7.632 | 18.694 | 1.00 | 14.10 |
| ATOM | 800 | CD1 | ILE | A | 634 | −3.757 | −9.128 | 18.829 | 1.00 | 14.44 |
| ATOM | 801 | C | ILE | A | 634 | −6.439 | −6.346 | 20.967 | 1.00 | 12.23 |
| ATOM | 802 | O | ILE | A | 634 | −6.456 | −6.894 | 22.067 | 1.00 | 12.06 |
| ATOM | 803 | N | GLN | A | 635 | −6.990 | −5.155 | 20.752 | 1.00 | 13.17 |
| ATOM | 804 | CA | GLN | A | 635 | −7.597 | −4.377 | 21.834 | 1.00 | 12.59 |
| ATOM | 805 | CB | GLN | A | 635 | −8.330 | −3.158 | 21.267 | 1.00 | 14.77 |
| ATOM | 806 | CG | GLN | A | 635 | −8.796 | −2.180 | 22.345 | 1.00 | 17.07 |
| ATOM | 807 | CD | GLN | A | 635 | −9.479 | −0.949 | 21.778 | 1.00 | 17.87 |
| ATOM | 808 | OE1 | GLN | A | 635 | −9.080 | −0.425 | 20.741 | 1.00 | 15.93 |
| ATOM | 809 | NE2 | GLN | A | 635 | −10.509 | −0.473 | 22.471 | 1.00 | 27.59 |
| ATOM | 810 | C | GLN | A | 635 | −8.549 | −5.127 | 22.762 | 1.00 | 13.46 |
| ATOM | 811 | O | GLN | A | 635 | −8.382 | −5.107 | 23.984 | 1.00 | 12.66 |
| ATOM | 812 | N | ASN | A | 636 | −9.554 | −5.778 | 22.188 | 1.00 | 12.36 |
| ATOM | 813 | CA | ASN | A | 636 | −10.533 | −6.493 | 22.996 | 1.00 | 15.16 |
| ATOM | 814 | CB | ASN | A | 636 | −11.736 | −6.872 | 22.130 | 1.00 | 13.54 |
| ATOM | 815 | CG | ASN | A | 636 | −12.508 | −5.655 | 21.658 | 1.00 | 19.96 |
| ATOM | 816 | OD1 | ASN | A | 636 | −12.211 | −4.526 | 22.059 | 1.00 | 19.43 |
| ATOM | 817 | ND2 | ASN | A | 636 | −13.506 | −5.874 | 20.810 | 1.00 | 20.88 |
| ATOM | 818 | C | ASN | A | 636 | −10.002 | −7.708 | 23.743 | 1.00 | 14.39 |
| ATOM | 819 | O | ASN | A | 636 | −10.720 | −8.312 | 24.539 | 1.00 | 17.61 |
| ATOM | 820 | N | LYS | A | 637 | −8.744 | −8.060 | 23.500 | 1.00 | 13.69 |
| ATOM | 821 | CA | LYS | A | 637 | −8.132 | −9.194 | 24.180 | 1.00 | 13.94 |
| ATOM | 822 | CB | LYS | A | 637 | −7.124 | −9.893 | 23.262 | 1.00 | 13.19 |
| ATOM | 823 | CG | LYS | A | 637 | −7.667 | −10.328 | 21.910 | 1.00 | 16.41 |
| ATOM | 824 | CD | LYS | A | 637 | −6.599 | −11.079 | 21.128 | 1.00 | 18.79 |
| ATOM | 825 | CE | LYS | A | 637 | −7.110 | −11.542 | 19.773 | 1.00 | 21.00 |
| ATOM | 826 | NZ | LYS | A | 637 | −8.266 | −12.467 | 19.893 | 1.00 | 27.72 |
| ATOM | 827 | C | LYS | A | 637 | −7.400 | −8.708 | 25.435 | 1.00 | 14.29 |
| ATOM | 828 | O | LYS | A | 637 | −6.930 | −9.514 | 26.237 | 1.00 | 15.91 |
| ATOM | 829 | N | LEU | A | 638 | −7.316 | −7.391 | 25.611 | 1.00 | 13.88 |
| ATOM | 830 | CA | LEU | A | 638 | −6.602 | −6.828 | 26.757 | 1.00 | 14.02 |
| ATOM | 831 | CB | LEU | A | 638 | −5.426 | −5.988 | 26.256 | 1.00 | 14.42 |
| ATOM | 832 | CG | LEU | A | 638 | −4.496 | −6.595 | 25.201 | 1.00 | 13.65 |
| ATOM | 833 | CD1 | LEU | A | 638 | −3.405 | −5.589 | 24.860 | 1.00 | 13.98 |
| ATOM | 834 | CD2 | LEU | A | 638 | −3.881 | −7.885 | 25.718 | 1.00 | 12.70 |
| ATOM | 835 | C | LEU | A | 638 | −7.464 | −5.973 | 27.687 | 1.00 | 13.83 |
| ATOM | 836 | O | LEU | A | 638 | −8.551 | −5.529 | 27.313 | 1.00 | 16.24 |
| ATOM | 837 | N | THR | A | 639 | −6.968 | −5.745 | 28.902 | 1.00 | 13.04 |
| ATOM | 838 | CA | THR | A | 639 | −7.665 | −4.913 | 29.885 | 1.00 | 12.97 |
| ATOM | 839 | CB | THR | A | 639 | −7.225 | −5.244 | 31.323 | 1.00 | 9.72 |
| ATOM | 840 | OG1 | THR | A | 639 | −5.883 | −4.777 | 31.534 | 1.00 | 11.58 |
| ATOM | 841 | CG2 | THR | A | 639 | −7.284 | −6.752 | 31.574 | 1.00 | 13.01 |
| ATOM | 842 | C | THR | A | 639 | −7.320 | −3.443 | 29.627 | 1.00 | 11.14 |
| ATOM | 843 | O | THR | A | 639 | −6.369 | −3.147 | 28.910 | 1.00 | 12.39 |
| ATOM | 844 | N | ASP | A | 640 | −8.082 | −2.524 | 30.216 | 1.00 | 11.83 |
| ATOM | 845 | CA | ASP | A | 640 | −7.814 | −1.098 | 30.027 | 1.00 | 11.03 |
| ATOM | 846 | CB | ASP | A | 640 | −8.846 | −0.243 | 30.774 | 1.00 | 9.58 |
| ATOM | 847 | CG | ASP | A | 640 | −10.238 | −0.368 | 30.191 | 1.00 | 23.27 |
| ATOM | 848 | OD1 | ASP | A | 640 | −10.357 | −0.828 | 29.037 | 1.00 | 21.74 |
| ATOM | 849 | OD2 | ASP | A | 640 | −11.211 | 0.011 | 30.879 | 1.00 | 18.96 |
| ATOM | 850 | C | ASP | A | 640 | −6.419 | −0.724 | 30.519 | 1.00 | 10.85 |
| ATOM | 851 | O | ASP | A | 640 | −5.708 | 0.058 | 29.881 | 1.00 | 11.05 |
| ATOM | 852 | N | LEU | A | 641 | −6.031 | −1.290 | 31.655 | 1.00 | 11.16 |
| ATOM | 853 | CA | LEU | A | 641 | −4.727 | −1.004 | 32.235 | 1.00 | 10.51 |
| ATOM | 854 | CB | LEU | A | 641 | −4.628 | −1.606 | 33.640 | 1.00 | 13.05 |
| ATOM | 855 | CG | LEU | A | 641 | −5.633 | −1.081 | 34.674 | 1.00 | 13.28 |
| ATOM | 856 | CD1 | LEU | A | 641 | −5.383 | −1.762 | 36.005 | 1.00 | 14.91 |
| ATOM | 857 | CD2 | LEU | A | 641 | −5.506 | 0.432 | 34.813 | 1.00 | 12.98 |
| ATOM | 858 | C | LEU | A | 641 | −3.593 | −1.527 | 31.359 | 1.00 | 10.02 |
| ATOM | 859 | O | LEU | A | 641 | −2.550 | −0.883 | 31.237 | 1.00 | 9.79 |
| ATOM | 860 | N | GLU | A | 642 | −3.791 | −2.692 | 30.748 | 1.00 | 9.49 |
| ATOM | 861 | CA | GLU | A | 642 | −2.756 | −3.253 | 29.886 | 1.00 | 9.55 |
| ATOM | 862 | CB | GLU | A | 642 | −3.120 | −4.687 | 29.493 | 1.00 | 13.24 |
| ATOM | 863 | CG | GLU | A | 642 | −3.215 | −5.605 | 30.705 | 1.00 | 17.56 |
| ATOM | 864 | CD | GLU | A | 642 | −3.645 | −7.021 | 30.373 | 1.00 | 14.47 |
| ATOM | 865 | OE1 | GLU | A | 642 | −4.411 | −7.212 | 29.404 | 1.00 | 12.63 |
| ATOM | 866 | OE2 | GLU | A | 642 | −3.233 | −7.949 | 31.108 | 1.00 | 15.36 |
| ATOM | 867 | C | GLU | A | 642 | −2.568 | −2.366 | 28.656 | 1.00 | 9.56 |
| ATOM | 868 | O | GLU | A | 642 | −1.442 | −2.135 | 28.217 | 1.00 | 10.36 |
| ATOM | 869 | N | ILE | A | 643 | −3.665 | −1.847 | 28.112 | 1.00 | 9.98 |
| ATOM | 870 | CA | ILE | A | 643 | −3.585 | −0.978 | 26.945 | 1.00 | 11.53 |
| ATOM | 871 | CB | ILE | A | 643 | −4.992 | −0.683 | 26.391 | 1.00 | 12.52 |
| ATOM | 872 | CG2 | ILE | A | 643 | −4.915 | 0.367 | 25.281 | 1.00 | 11.83 |
| ATOM | 873 | CG1 | ILE | A | 643 | −5.614 | −1.984 | 25.885 | 1.00 | 11.82 |
| ATOM | 874 | CD1 | ILE | A | 643 | −7.089 | −1.872 | 25.553 | 1.00 | 18.49 |
| ATOM | 875 | C | ILE | A | 643 | −2.883 | 0.323 | 27.339 | 1.00 | 9.56 |
| ATOM | 876 | O | ILE | A | 643 | −1.993 | 0.806 | 26.635 | 1.00 | 10.76 |
| ATOM | 877 | N | LEU | A | 644 | −3.278 | 0.880 | 28.479 | 1.00 | 9.68 |
| ATOM | 878 | CA | LEU | A | 644 | −2.663 | 2.105 | 28.974 | 1.00 | 9.78 |
| ATOM | 879 | CB | LEU | A | 644 | −3.285 | 2.482 | 30.321 | 1.00 | 11.15 |
| ATOM | 880 | CG | LEU | A | 644 | −2.658 | 3.643 | 31.093 | 1.00 | 14.02 |
| ATOM | 881 | CD1 | LEU | A | 644 | −2.853 | 4.946 | 30.328 | 1.00 | 14.15 |
| ATOM | 882 | CD2 | LEU | A | 644 | −3.315 | 3.735 | 32.471 | 1.00 | 14.10 |
| ATOM | 883 | C | LEU | A | 644 | −1.152 | 1.899 | 29.139 | 1.00 | 9.98 |
| ATOM | 884 | O | LEU | A | 644 | −0.342 | 2.720 | 28.694 | 1.00 | 9.97 |
| ATOM | 885 | N | ALA | A | 645 | −0.778 | 0.788 | 29.766 | 1.00 | 8.98 |
| ATOM | 886 | CA | ALA | A | 645 | 0.634 | 0.492 | 30.008 | 1.00 | 8.84 |
| ATOM | 887 | CB | ALA | A | 645 | 0.763 | −0.724 | 30.928 | 1.00 | 8.17 |
| ATOM | 888 | C | ALA | A | 645 | 1.414 | 0.256 | 28.718 | 1.00 | 9.39 |
| ATOM | 889 | O | ALA | A | 645 | 2.556 | 0.687 | 28.598 | 1.00 | 9.92 |
| ATOM | 890 | N | LEU | A | 646 | 0.799 | −0.430 | 27.761 | 1.00 | 8.76 |
| ATOM | 891 | CA | LEU | A | 646 | 1.462 | −0.708 | 26.488 | 1.00 | 8.34 |
| ATOM | 892 | CB | LEU | A | 646 | 0.607 | −1.652 | 25.644 | 1.00 | 10.30 |
| ATOM | 893 | CG | LEU | A | 646 | 0.541 | −3.101 | 26.128 | 1.00 | 11.47 |
| ATOM | 894 | CD1 | LEU | A | 646 | −0.557 | −3.832 | 25.379 | 1.00 | 15.28 |
| ATOM | 895 | CD2 | LEU | A | 646 | 1.879 | −3.779 | 25.906 | 1.00 | 10.48 |
| ATOM | 896 | C | LEU | A | 646 | 1.761 | 0.550 | 25.682 | 1.00 | 7.55 |
| ATOM | 897 | O | LEU | A | 646 | 2.837 | 0.676 | 25.094 | 1.00 | 9.05 |
| ATOM | 898 | N | LEU | A | 647 | 0.812 | 1.479 | 25.643 | 1.00 | 7.57 |
| ATOM | 899 | CA | LEU | A | 647 | 1.020 | 2.713 | 24.889 | 1.00 | 8.87 |
| ATOM | 900 | CB | LEU | A | 647 | −0.283 | 3.516 | 24.803 | 1.00 | 7.82 |
| ATOM | 901 | CG | LEU | A | 647 | −0.214 | 4.722 | 23.862 | 1.00 | 10.06 |
| ATOM | 902 | CD1 | LEU | A | 647 | 0.194 | 4.251 | 22.470 | 1.00 | 13.81 |
| ATOM | 903 | CD2 | LEU | A | 647 | −1.560 | 5.437 | 23.820 | 1.00 | 12.05 |
| ATOM | 904 | C | LEU | A | 647 | 2.120 | 3.550 | 25.540 | 1.00 | 8.12 |
| ATOM | 905 | O | LEU | A | 647 | 3.011 | 4.070 | 24.863 | 1.00 | 8.78 |
| ATOM | 906 | N | ILE | A | 648 | 2.056 | 3.676 | 26.860 | 1.00 | 9.66 |
| ATOM | 907 | CA | ILE | A | 648 | 3.065 | 4.429 | 27.597 | 1.00 | 8.86 |
| ATOM | 908 | CB | ILE | A | 648 | 2.692 | 4.501 | 29.100 | 1.00 | 10.81 |
| ATOM | 909 | CG2 | ILE | A | 648 | 3.851 | 5.063 | 29.917 | 1.00 | 7.70 |
| ATOM | 910 | CG1 | ILE | A | 648 | 1.439 | 5.364 | 29.266 | 1.00 | 11.45 |
| ATOM | 911 | CD1 | ILE | A | 648 | 0.921 | 5.437 | 30.688 | 1.00 | 9.57 |
| ATOM | 912 | C | ILE | A | 648 | 4.427 | 3.756 | 27.424 | 1.00 | 8.38 |
| ATOM | 913 | O | ILE | A | 648 | 5.439 | 4.427 | 27.180 | 1.00 | 9.63 |
| ATOM | 914 | N | ALA | A | 649 | 4.456 | 2.430 | 27.536 | 1.00 | 9.32 |
| ATOM | 915 | CA | ALA | A | 649 | 5.710 | 1.697 | 27.388 | 1.00 | 9.98 |
| ATOM | 916 | CB | ALA | A | 649 | 5.506 | 0.214 | 27.703 | 1.00 | 11.64 |
| ATOM | 917 | C | ALA | A | 649 | 6.285 | 1.853 | 25.989 | 1.00 | 9.64 |
| ATOM | 918 | O | ALA | A | 649 | 7.471 | 2.126 | 25.831 | 1.00 | 10.34 |
| ATOM | 919 | N | ALA | A | 650 | 5.449 | 1.680 | 24.971 | 1.00 | 11.02 |
| ATOM | 920 | CA | ALA | A | 650 | 5.914 | 1.814 | 23.591 | 1.00 | 9.43 |
| ATOM | 921 | CB | ALA | A | 650 | 4.747 | 1.618 | 22.617 | 1.00 | 8.79 |
| ATOM | 922 | C | ALA | A | 650 | 6.561 | 3.183 | 23.359 | 1.00 | 8.11 |
| ATOM | 923 | O | ALA | A | 650 | 7.628 | 3.292 | 22.748 | 1.00 | 9.60 |
| ATOM | 924 | N | LEU | A | 651 | 5.914 | 4.233 | 23.851 | 1.00 | 9.58 |
| ATOM | 925 | CA | LEU | A | 651 | 6.443 | 5.582 | 23.679 | 1.00 | 10.27 |
| ATOM | 926 | CB | LEU | A | 651 | 5.383 | 6.613 | 24.066 | 1.00 | 9.84 |
| ATOM | 927 | CG | LEU | A | 651 | 4.198 | 6.717 | 23.101 | 1.00 | 6.24 |
| ATOM | 928 | CD1 | LEU | A | 651 | 3.038 | 7.432 | 23.772 | 1.00 | 14.57 |
| ATOM | 929 | CD2 | LEU | A | 651 | 4.637 | 7.463 | 21.846 | 1.00 | 11.43 |
| ATOM | 930 | C | LEU | A | 651 | 7.706 | 5.848 | 24.488 | 1.00 | 10.46 |
| ATOM | 931 | O | LEU | A | 651 | 8.573 | 6.611 | 24.066 | 1.00 | 9.75 |
| ATOM | 932 | N | SER | A | 652 | 7.807 | 5.194 | 25.640 | 1.00 | 9.58 |
| ATOM | 933 | CA | SER | A | 652 | 8.935 | 5.394 | 26.542 | 1.00 | 10.30 |
| ATOM | 934 | CB | SER | A | 652 | 8.422 | 5.453 | 27.983 | 1.00 | 9.30 |
| ATOM | 935 | OG | SER | A | 652 | 7.367 | 6.379 | 28.133 | 1.00 | 11.78 |
| ATOM | 936 | C | SER | A | 652 | 10.026 | 4.334 | 26.504 | 1.00 | 8.64 |
| ATOM | 937 | O | SER | A | 652 | 11.068 | 4.520 | 27.125 | 1.00 | 9.90 |
| ATOM | 938 | N | HIS | A | 653 | 9.805 | 3.249 | 25.765 | 1.00 | 9.64 |
| ATOM | 939 | CA | HIS | A | 653 | 10.739 | 2.128 | 25.769 | 1.00 | 9.99 |
| ATOM | 940 | CB | HIS | A | 653 | 10.164 | 0.963 | 24.952 | 1.00 | 8.26 |
| ATOM | 941 | CG | HIS | A | 653 | 10.546 | 0.980 | 23.510 | 1.00 | 8.55 |
| ATOM | 942 | CD2 | HIS | A | 653 | 11.529 | 0.333 | 22.842 | 1.00 | 9.53 |
| ATOM | 943 | ND1 | HIS | A | 653 | 9.881 | 1.740 | 22.569 | 1.00 | 10.01 |
| ATOM | 944 | CE1 | HIS | A | 653 | 10.440 | 1.559 | 21.387 | 1.00 | 6.46 |
| ATOM | 945 | NE2 | HIS | A | 653 | 11.444 | 0.708 | 21.526 | 1.00 | 6.65 |
| ATOM | 946 | C | HIS | A | 653 | 12.210 | 2.321 | 25.412 | 1.00 | 10.62 |
| ATOM | 947 | O | HIS | A | 653 | 13.024 | 1.459 | 25.741 | 1.00 | 8.86 |
| ATOM | 948 | N | ASP | A | 654 | 12.559 | 3.424 | 24.752 | 1.00 | 9.82 |
| ATOM | 949 | CA | ASP | A | 654 | 13.959 | 3.673 | 24.406 | 1.00 | 9.46 |
| ATOM | 950 | CB | ASP | A | 654 | 14.184 | 3.580 | 22.889 | 1.00 | 10.10 |
| ATOM | 951 | CG | ASP | A | 654 | 14.509 | 2.172 | 22.432 | 1.00 | 10.77 |
| ATOM | 952 | OD1 | ASP | A | 654 | 15.159 | 1.444 | 23.204 | 1.00 | 9.55 |
| ATOM | 953 | OD2 | ASP | A | 654 | 14.141 | 1.812 | 21.294 | 1.00 | 8.85 |
| ATOM | 954 | C | ASP | A | 654 | 14.477 | 5.024 | 24.896 | 1.00 | 9.92 |
| ATOM | 955 | O | ASP | A | 654 | 15.500 | 5.507 | 24.413 | 1.00 | 9.67 |
| ATOM | 956 | N | LEU | A | 655 | 13.783 | 5.619 | 25.865 | 1.00 | 9.19 |
| ATOM | 957 | CA | LEU | A | 655 | 14.169 | 6.922 | 26.408 | 1.00 | 10.06 |
| ATOM | 958 | CB | LEU | A | 655 | 13.364 | 7.214 | 27.677 | 1.00 | 8.55 |
| ATOM | 959 | CG | LEU | A | 655 | 11.900 | 7.603 | 27.479 | 1.00 | 10.24 |
| ATOM | 960 | CD1 | LEU | A | 655 | 11.151 | 7.468 | 28.805 | 1.00 | 9.10 |
| ATOM | 961 | CD2 | LEU | A | 655 | 11.819 | 9.028 | 26.944 | 1.00 | 15.71 |
| ATOM | 962 | C | LEU | A | 655 | 15.658 | 7.101 | 26.713 | 1.00 | 10.50 |
| ATOM | 963 | O | LEU | A | 655 | 16.279 | 6.268 | 27.372 | 1.00 | 9.92 |
| ATOM | 964 | N | ASP | A | 656 | 16.215 | 8.209 | 26.232 | 1.00 | 8.66 |
| ATOM | 965 | CA | ASP | A | 656 | 17.622 | 8.542 | 26.448 | 1.00 | 8.66 |
| ATOM | 966 | CB | ASP | A | 656 | 17.867 | 8.811 | 27.941 | 1.00 | 11.08 |
| ATOM | 967 | CG | ASP | A | 656 | 19.211 | 9.475 | 28.207 | 1.00 | 16.07 |
| ATOM | 968 | OD1 | ASP | A | 656 | 19.576 | 10.406 | 27.460 | 1.00 | 11.04 |
| ATOM | 969 | OD2 | ASP | A | 656 | 19.897 | 9.074 | 29.173 | 1.00 | 13.88 |
| ATOM | 970 | C | ASP | A | 656 | 18.600 | 7.483 | 25.939 | 1.00 | 8.59 |
| ATOM | 971 | O | ASP | A | 656 | 19.670 | 7.288 | 26.511 | 1.00 | 9.89 |
| ATOM | 972 | N | HIS | A | 657 | 18.242 | 6.806 | 24.851 | 1.00 | 9.07 |
| ATOM | 973 | CA | HIS | A | 657 | 19.118 | 5.790 | 24.284 | 1.00 | 10.63 |
| ATOM | 974 | CB | HIS | A | 657 | 18.412 | 5.068 | 23.137 | 1.00 | 9.06 |
| ATOM | 975 | CG | HIS | A | 657 | 19.095 | 3.810 | 22.712 | 1.00 | 8.47 |
| ATOM | 976 | CD2 | HIS | A | 657 | 20.347 | 3.589 | 22.245 | 1.00 | 7.97 |
| ATOM | 977 | ND1 | HIS | A | 657 | 18.480 | 2.575 | 22.748 | 1.00 | 12.46 |
| ATOM | 978 | CE1 | HIS | A | 657 | 19.322 | 1.652 | 22.322 | 1.00 | 5.38 |
| ATOM | 979 | NE2 | HIS | A | 657 | 20.464 | 2.242 | 22.011 | 1.00 | 12.33 |
| ATOM | 980 | C | HIS | A | 657 | 20.404 | 6.453 | 23.778 | 1.00 | 11.40 |
| ATOM | 981 | O | HIS | A | 657 | 20.362 | 7.404 | 22.993 | 1.00 | 11.45 |
| ATOM | 982 | N | PRO | A | 658 | 21.567 | 5.957 | 24.230 | 1.00 | 11.01 |
| ATOM | 983 | CD | PRO | A | 658 | 21.697 | 4.963 | 25.311 | 1.00 | 10.94 |
| ATOM | 984 | CA | PRO | A | 658 | 22.884 | 6.479 | 23.850 | 1.00 | 10.19 |
| ATOM | 985 | CB | PRO | A | 658 | 23.792 | 5.930 | 24.944 | 1.00 | 9.66 |
| ATOM | 986 | CG | PRO | A | 658 | 23.171 | 4.618 | 25.252 | 1.00 | 16.61 |
| ATOM | 987 | C | PRO | A | 658 | 23.386 | 6.123 | 22.451 | 1.00 | 11.02 |
| ATOM | 988 | O | PRO | A | 658 | 24.428 | 6.627 | 22.021 | 1.00 | 12.41 |
| ATOM | 989 | N | GLY | A | 659 | 22.659 | 5.265 | 21.744 | 1.00 | 8.82 |
| ATOM | 990 | CA | GLY | A | 659 | 23.080 | 4.888 | 20.405 | 1.00 | 8.78 |
| ATOM | 991 | C | GLY | A | 659 | 24.115 | 3.781 | 20.356 | 1.00 | 9.97 |
| ATOM | 992 | O | GLY | A | 659 | 24.750 | 3.566 | 19.316 | 1.00 | 12.32 |
| ATOM | 993 | N | VAL | A | 660 | 24.305 | 3.095 | 21.480 | 1.00 | 8.93 |
| ATOM | 994 | CA | VAL | A | 660 | 25.248 | 1.983 | 21.564 | 1.00 | 9.69 |
| ATOM | 995 | CB | VAL | A | 660 | 26.554 | 2.387 | 22.301 | 1.00 | 10.19 |
| ATOM | 996 | CG1 | VAL | A | 660 | 27.358 | 3.355 | 21.434 | 1.00 | 12.07 |
| ATOM | 997 | CG2 | VAL | A | 660 | 26.236 | 3.020 | 23.642 | 1.00 | 12.93 |
| ATOM | 998 | C | VAL | A | 660 | 24.545 | 0.834 | 22.289 | 1.00 | 10.76 |
| ATOM | 999 | O | VAL | A | 660 | 23.601 | 1.059 | 23.052 | 1.00 | 10.23 |
| ATOM | 1000 | N | SER | A | 661 | 25.002 | −0.392 | 22.049 | 1.00 | 7.63 |
| ATOM | 1001 | CA | SER | A | 661 | 24.371 | −1.577 | 22.624 | 1.00 | 10.33 |
| ATOM | 1002 | CB | SER | A | 661 | 24.748 | −2.801 | 21.793 | 1.00 | 8.76 |
| ATOM | 1003 | OG | SER | A | 661 | 26.099 | −3.151 | 22.021 | 1.00 | 9.56 |
| ATOM | 1004 | C | SER | A | 661 | 24.687 | −1.864 | 24.088 | 1.00 | 9.36 |
| ATOM | 1005 | O | SER | A | 661 | 25.549 | −1.230 | 24.696 | 1.00 | 10.42 |
| ATOM | 1006 | N | ASN | A | 662 | 23.969 | −2.838 | 24.643 | 1.00 | 10.10 |
| ATOM | 1007 | CA | ASN | A | 662 | 24.165 | −3.261 | 26.026 | 1.00 | 8.94 |
| ATOM | 1008 | CB | ASN | A | 662 | 23.177 | −4.369 | 26.384 | 1.00 | 12.24 |
| ATOM | 1009 | CG | ASN | A | 662 | 21.832 | −3.836 | 26.824 | 1.00 | 11.27 |
| ATOM | 1010 | OD1 | ASN | A | 662 | 20.794 | −4.457 | 26.592 | 1.00 | 19.20 |
| ATOM | 1011 | ND2 | ASN | A | 662 | 21.844 | −2.689 | 27.483 | 1.00 | 5.90 |
| ATOM | 1012 | C | ASN | A | 662 | 25.586 | −3.781 | 26.161 | 1.00 | 11.89 |
| ATOM | 1013 | O | ASN | A | 662 | 26.289 | −3.459 | 27.116 | 1.00 | 12.39 |
| ATOM | 1014 | N | GLN | A | 663 | 26.010 | −4.598 | 25.199 | 1.00 | 10.34 |
| ATOM | 1015 | CA | GLN | A | 663 | 27.356 | −5.139 | 25.246 | 1.00 | 11.06 |
| ATOM | 1016 | CB | GLN | A | 663 | 27.578 | −6.120 | 24.083 | 1.00 | 14.83 |
| ATOM | 1017 | CG | GLN | A | 663 | 26.677 | −7.373 | 24.197 | 1.00 | 25.28 |
| ATOM | 1018 | CD | GLN | A | 663 | 25.283 | −7.191 | 23.575 | 1.00 | 24.96 |
| ATOM | 1019 | OE1 | GLN | A | 663 | 24.919 | −6.113 | 23.106 | 1.00 | 31.10 |
| ATOM | 1020 | NE2 | GLN | A | 663 | 24.497 | −8.271 | 23.572 | 1.00 | 46.85 |
| ATOM | 1021 | C | GLN | A | 663 | 28.422 | −4.039 | 25.236 | 1.00 | 13.51 |
| ATOM | 1022 | O | GLN | A | 663 | 29.439 | −4.161 | 25.921 | 1.00 | 12.70 |
| ATOM | 1023 | N | PHE | A | 664 | 28.184 | −2.956 | 24.504 | 1.00 | 12.64 |
| ATOM | 1024 | CA | PHE | A | 664 | 29.145 | −1.852 | 24.465 | 1.00 | 11.20 |
| ATOM | 1025 | CB | PHE | A | 664 | 28.754 | −0.838 | 23.380 | 1.00 | 12.60 |
| ATOM | 1026 | CG | PHE | A | 664 | 29.669 | 0.355 | 23.296 | 1.00 | 15.21 |
| ATOM | 1027 | CD1 | PHE | A | 664 | 29.554 | 1.407 | 24.198 | 1.00 | 13.93 |
| ATOM | 1028 | CD2 | PHE | A | 664 | 30.668 | 0.409 | 22.336 | 1.00 | 10.34 |
| ATOM | 1029 | CE1 | PHE | A | 664 | 30.419 | 2.484 | 24.144 | 1.00 | 11.59 |
| ATOM | 1030 | CE2 | PHE | A | 664 | 31.541 | 1.487 | 22.275 | 1.00 | 16.42 |
| ATOM | 1031 | CZ | PHE | A | 664 | 31.418 | 2.527 | 23.182 | 1.00 | 18.27 |
| ATOM | 1032 | C | PHE | A | 664 | 29.207 | −1.171 | 25.830 | 1.00 | 11.75 |
| ATOM | 1033 | O | PHE | A | 664 | 30.290 | −0.818 | 26.306 | 1.00 | 11.28 |
| ATOM | 1034 | N | LEU | A | 665 | 28.046 | −0.980 | 26.452 | 1.00 | 11.17 |
| ATOM | 1035 | CA | LEU | A | 665 | 27.982 | −0.346 | 27.768 | 1.00 | 11.46 |
| ATOM | 1036 | CB | LEU | A | 665 | 26.524 | −0.163 | 28.204 | 1.00 | 11.38 |
| ATOM | 1037 | CG | LEU | A | 665 | 25.703 | 0.864 | 27.420 | 1.00 | 9.72 |
| ATOM | 1038 | CD1 | LEU | A | 665 | 24.304 | 0.959 | 28.015 | 1.00 | 16.35 |
| ATOM | 1039 | CD2 | LEU | A | 665 | 26.399 | 2.215 | 27.470 | 1.00 | 13.26 |
| ATOM | 1040 | C | LEU | A | 665 | 28.726 | −1.200 | 28.793 | 1.00 | 13.75 |
| ATOM | 1041 | O | LEU | A | 665 | 29.404 | −0.686 | 29.681 | 1.00 | 11.85 |
| ATOM | 1042 | N | ILE | A | 666 | 28.591 | −2.512 | 28.661 | 1.00 | 12.41 |
| ATOM | 1043 | CA | ILE | A | 666 | 29.259 | −3.444 | 29.559 | 1.00 | 13.52 |
| ATOM | 1044 | CB | ILE | A | 666 | 28.773 | −4.887 | 29.300 | 1.00 | 13.10 |
| ATOM | 1045 | CG2 | ILE | A | 666 | 29.667 | −5.882 | 30.020 | 1.00 | 13.00 |
| ATOM | 1046 | CG1 | ILE | A | 666 | 27.322 | −5.036 | 29.761 | 1.00 | 10.73 |
| ATOM | 1047 | CD1 | ILE | A | 666 | 26.687 | −6.346 | 29.369 | 1.00 | 14.67 |
| ATOM | 1048 | C | ILE | A | 666 | 30.770 | −3.381 | 29.356 | 1.00 | 11.71 |
| ATOM | 1049 | O | ILE | A | 666 | 31.541 | −3.297 | 30.317 | 1.00 | 14.40 |
| ATOM | 1050 | N | ASN | A | 667 | 31.183 | −3.408 | 28.094 | 1.00 | 12.52 |
| ATOM | 1051 | CA | ASN | A | 667 | 32.599 | −3.382 | 27.733 | 1.00 | 11.69 |
| ATOM | 1052 | CB | ASN | A | 667 | 32.745 | −3.652 | 26.232 | 1.00 | 15.94 |
| ATOM | 1053 | CG | ASN | A | 667 | 32.340 | −5.065 | 25.848 | 1.00 | 13.91 |
| ATOM | 1054 | OD1 | ASN | A | 667 | 32.138 | −5.363 | 24.669 | 1.00 | 17.63 |
| ATOM | 1055 | ND2 | ASN | A | 667 | 32.233 | −5.946 | 26.837 | 1.00 | 11.25 |
| ATOM | 1056 | C | ASN | A | 667 | 33.323 | −2.090 | 28.091 | 1.00 | 15.59 |
| ATOM | 1057 | O | ASN | A | 667 | 34.530 | −2.104 | 28.355 | 1.00 | 16.72 |
| ATOM | 1058 | N | THR | A | 668 | 32.601 | −0.974 | 28.098 | 1.00 | 15.05 |
| ATOM | 1059 | CA | THR | A | 668 | 33.211 | 0.308 | 28.428 | 1.00 | 16.29 |
| ATOM | 1060 | CB | THR | A | 668 | 32.670 | 1.432 | 27.520 | 1.00 | 16.12 |
| ATOM | 1061 | OG1 | THR | A | 668 | 31.237 | 1.448 | 27.567 | 1.00 | 19.49 |
| ATOM | 1062 | CG2 | THR | A | 668 | 33.133 | 1.207 | 26.078 | 1.00 | 15.93 |
| ATOM | 1063 | C | THR | A | 668 | 32.995 | 0.672 | 29.895 | 1.00 | 18.10 |
| ATOM | 1064 | O | THR | A | 668 | 33.158 | 1.830 | 30.297 | 1.00 | 20.65 |
| ATOM | 1065 | N | ASN | A | 669 | 32.617 | −0.330 | 30.682 | 1.00 | 17.75 |
| ATOM | 1066 | CA | ASN | A | 669 | 32.402 | −0.177 | 32.115 | 1.00 | 20.15 |
| ATOM | 1067 | CB | ASN | A | 669 | 33.749 | 0.053 | 32.797 | 1.00 | 28.92 |
| ATOM | 1068 | CG | ASN | A | 669 | 34.791 | −0.955 | 32.361 | 1.00 | 34.79 |
| ATOM | 1069 | OD1 | ASN | A | 669 | 34.619 | −2.160 | 32.543 | 1.00 | 40.75 |
| ATOM | 1070 | ND2 | ASN | A | 669 | 35.877 | −0.468 | 31.772 | 1.00 | 36.76 |
| ATOM | 1071 | C | ASN | A | 669 | 31.428 | 0.926 | 32.515 | 1.00 | 20.87 |
| ATOM | 1072 | O | ASN | A | 669 | 31.690 | 1.696 | 33.443 | 1.00 | 21.59 |
| ATOM | 1073 | N | SER | A | 670 | 30.302 | 0.996 | 31.819 | 1.00 | 17.34 |
| ATOM | 1074 | CA | SER | A | 670 | 29.286 | 1.992 | 32.120 | 1.00 | 19.60 |
| ATOM | 1075 | CB | SER | A | 670 | 28.103 | 1.836 | 31.160 | 1.00 | 18.96 |
| ATOM | 1076 | OG | SER | A | 670 | 27.023 | 2.672 | 31.535 | 1.00 | 22.69 |
| ATOM | 1077 | C | SER | A | 670 | 28.803 | 1.787 | 33.551 | 1.00 | 16.24 |
| ATOM | 1078 | O | SER | A | 670 | 28.755 | 0.655 | 34.041 | 1.00 | 14.46 |
| ATOM | 1079 | N | GLU | A | 671 | 28.455 | 2.879 | 34.227 | 1.00 | 18.45 |
| ATOM | 1080 | CA | GLU | A | 671 | 27.951 | 2.775 | 35.589 | 1.00 | 19.69 |
| ATOM | 1081 | CB | GLU | A | 671 | 27.756 | 4.154 | 36.209 | 1.00 | 17.48 |
| ATOM | 1082 | CG | GLU | A | 671 | 28.985 | 5.024 | 36.189 | 1.00 | 31.14 |
| ATOM | 1083 | CD | GLU | A | 671 | 28.901 | 6.156 | 37.191 | 1.00 | 32.78 |
| ATOM | 1084 | OE1 | GLU | A | 671 | 27.830 | 6.796 | 37.291 | 1.00 | 31.62 |
| ATOM | 1085 | OE2 | GLU | A | 671 | 29.914 | 6.406 | 37.875 | 1.00 | 26.52 |
| ATOM | 1086 | C | GLU | A | 671 | 26.611 | 2.058 | 35.549 | 1.00 | 18.77 |
| ATOM | 1087 | O | GLU | A | 671 | 26.253 | 1.332 | 36.482 | 1.00 | 19.75 |
| ATOM | 1088 | N | LEU | A | 672 | 25.868 | 2.281 | 34.466 | 1.00 | 17.92 |
| ATOM | 1089 | CA | LEU | A | 672 | 24.569 | 1.646 | 34.279 | 1.00 | 15.76 |
| ATOM | 1090 | CB | LEU | A | 672 | 23.954 | 2.067 | 32.942 | 1.00 | 12.50 |
| ATOM | 1091 | CG | LEU | A | 672 | 23.478 | 3.509 | 32.771 | 1.00 | 18.92 |
| ATOM | 1092 | CD1 | LEU | A | 672 | 23.101 | 3.741 | 31.314 | 1.00 | 18.65 |
| ATOM | 1093 | CD2 | LEU | A | 672 | 22.292 | 3.766 | 33.678 | 1.00 | 24.52 |
| ATOM | 1094 | C | LEU | A | 672 | 24.738 | 0.130 | 34.291 | 1.00 | 13.50 |
| ATOM | 1095 | O | LEU | A | 672 | 23.961 | −0.586 | 34.912 | 1.00 | 12.92 |
| ATOM | 1096 | N | ALA | A | 673 | 25.755 | −0.358 | 33.590 | 1.00 | 13.11 |
| ATOM | 1097 | CA | ALA | A | 673 | 26.001 | −1.792 | 33.546 | 1.00 | 13.53 |
| ATOM | 1098 | CB | ALA | A | 673 | 27.169 | −2.099 | 32.594 | 1.00 | 11.10 |
| ATOM | 1099 | C | ALA | A | 673 | 26.318 | −2.283 | 34.956 | 1.00 | 13.78 |
| ATOM | 1100 | O | ALA | A | 673 | 25.925 | −3.380 | 35.354 | 1.00 | 12.36 |
| ATOM | 1101 | N | LEU | A | 674 | 27.028 | −1.458 | 35.718 | 1.00 | 15.22 |
| ATOM | 1102 | CA | LEU | A | 674 | 27.394 | −1.829 | 37.076 | 1.00 | 16.65 |
| ATOM | 1103 | CB | LEU | A | 674 | 28.414 | −0.832 | 37.632 | 1.00 | 18.96 |
| ATOM | 1104 | CG | LEU | A | 674 | 29.225 | −1.323 | 38.832 | 1.00 | 26.52 |
| ATOM | 1105 | CD1 | LEU | A | 674 | 29.939 | −2.620 | 38.460 | 1.00 | 24.45 |
| ATOM | 1106 | CD2 | LEU | A | 674 | 30.231 | −0.258 | 39.246 | 1.00 | 20.57 |
| ATOM | 1107 | C | LEU | A | 674 | 26.152 | −1.866 | 37.960 | 1.00 | 15.94 |
| ATOM | 1108 | O | LEU | A | 674 | 25.930 | −2.818 | 38.709 | 1.00 | 16.86 |
| ATOM | 1109 | N | MET | A | 675 | 25.335 | −0.822 | 37.851 | 1.00 | 19.35 |
| ATOM | 1110 | CA | MET | A | 675 | 24.107 | −0.694 | 38.627 | 1.00 | 18.57 |
| ATOM | 1111 | CB | MET | A | 675 | 23.413 | 0.641 | 38.312 | 1.00 | 29.02 |
| ATOM | 1112 | CG | MET | A | 675 | 24.180 | 1.882 | 38.753 | 1.00 | 32.86 |
| ATOM | 1113 | SD | MET | A | 675 | 23.766 | 3.373 | 37.785 | 1.00 | 38.23 |
| ATOM | 1114 | CE | MET | A | 675 | 22.129 | 3.769 | 38.381 | 1.00 | 40.96 |
| ATOM | 1115 | C | MET | A | 675 | 23.133 | −1.830 | 38.346 | 1.00 | 17.83 |
| ATOM | 1116 | O | MET | A | 675 | 22.529 | −2.383 | 39.265 | 1.00 | 17.84 |
| ATOM | 1117 | N | TYR | A | 676 | 23.000 | −2.184 | 37.073 | 1.00 | 15.75 |
| ATOM | 1118 | CA | TYR | A | 676 | 22.052 | −3.212 | 36.679 | 1.00 | 16.85 |
| ATOM | 1119 | CB | TYR | A | 676 | 21.293 | −2.720 | 35.442 | 1.00 | 16.19 |
| ATOM | 1120 | CG | TYR | A | 676 | 20.619 | −1.374 | 35.668 | 1.00 | 20.30 |
| ATOM | 1121 | CD1 | TYR | A | 676 | 19.694 | −1.197 | 36.691 | 1.00 | 31.51 |
| ATOM | 1122 | CE1 | TYR | A | 676 | 19.107 | 0.045 | 36.922 | 1.00 | 18.89 |
| ATOM | 1123 | CD2 | TYR | A | 676 | 20.934 | −0.276 | 34.877 | 1.00 | 23.78 |
| ATOM | 1124 | CE2 | TYR | A | 676 | 20.354 | 0.962 | 35.097 | 1.00 | 27.90 |
| ATOM | 1125 | CZ | TYR | A | 676 | 19.443 | 1.117 | 36.118 | 1.00 | 24.78 |
| ATOM | 1126 | OH | TYR | A | 676 | 18.866 | 2.354 | 36.326 | 1.00 | 28.03 |
| ATOM | 1127 | C | TYR | A | 676 | 22.605 | −4.623 | 36.471 | 1.00 | 16.48 |
| ATOM | 1128 | O | TYR | A | 676 | 21.949 | −5.478 | 35.872 | 1.00 | 15.88 |
| ATOM | 1129 | N | ASN | A | 677 | 23.814 | −4.863 | 36.968 | 1.00 | 16.14 |
| ATOM | 1130 | CA | ASN | A | 677 | 24.433 | −6.182 | 36.888 | 1.00 | 14.23 |
| ATOM | 1131 | CB | ASN | A | 677 | 23.666 | −7.138 | 37.812 | 1.00 | 21.33 |
| ATOM | 1132 | CG | ASN | A | 677 | 24.382 | −8.452 | 38.022 | 1.00 | 29.37 |
| ATOM | 1133 | OD1 | ASN | A | 677 | 25.570 | −8.478 | 38.349 | 1.00 | 38.44 |
| ATOM | 1134 | ND2 | ASN | A | 677 | 23.662 | −9.555 | 37.847 | 1.00 | 42.62 |
| ATOM | 1135 | C | ASN | A | 677 | 24.516 | −6.765 | 35.474 | 1.00 | 15.05 |
| ATOM | 1136 | O | ASN | A | 677 | 24.332 | −7.966 | 35.275 | 1.00 | 14.59 |
| ATOM | 1137 | N | ASP | A | 678 | 24.802 | −5.904 | 34.502 | 1.00 | 13.69 |
| ATOM | 1138 | CA | ASP | A | 678 | 24.926 | −6.302 | 33.100 | 1.00 | 16.08 |
| ATOM | 1139 | CB | ASP | A | 678 | 26.080 | −7.293 | 32.915 | 1.00 | 12.96 |
| ATOM | 1140 | CG | ASP | A | 678 | 27.430 | −6.691 | 33.234 | 1.00 | 18.27 |
| ATOM | 1141 | OD1 | ASP | A | 678 | 27.519 | −5.452 | 33.377 | 1.00 | 16.21 |
| ATOM | 1142 | OD2 | ASP | A | 678 | 28.406 | −7.464 | 33.334 | 1.00 | 20.95 |
| ATOM | 1143 | C | ASP | A | 678 | 23.667 | −6.919 | 32.499 | 1.00 | 16.35 |
| ATOM | 1144 | O | ASP | A | 678 | 23.731 | −7.509 | 31.420 | 1.00 | 17.49 |
| ATOM | 1145 | N | GLU | A | 679 | 22.534 | −6.790 | 33.184 | 1.00 | 14.03 |
| ATOM | 1146 | CA | GLU | A | 679 | 21.278 | −7.358 | 32.690 | 1.00 | 14.39 |
| ATOM | 1147 | CB | GLU | A | 679 | 20.567 | −8.133 | 33.799 | 1.00 | 14.04 |
| ATOM | 1148 | CG | GLU | A | 679 | 21.314 | −9.351 | 34.300 | 1.00 | 22.79 |
| ATOM | 1149 | CD | GLU | A | 679 | 20.514 | −10.128 | 35.330 | 1.00 | 43.42 |
| ATOM | 1150 | OE1 | GLU | A | 679 | 19.464 | −10.699 | 34.964 | 1.00 | 51.30 |
| ATOM | 1151 | OE2 | GLU | A | 679 | 20.931 | −10.162 | 36.507 | 1.00 | 41.34 |
| ATOM | 1152 | C | GLU | A | 679 | 20.320 | −6.301 | 32.151 | 1.00 | 12.89 |
| ATOM | 1153 | O | GLU | A | 679 | 19.873 | −5.430 | 32.895 | 1.00 | 13.61 |
| ATOM | 1154 | N | SER | A | 680 | 19.993 | −6.401 | 30.863 | 1.00 | 10.87 |
| ATOM | 1155 | CA | SER | A | 680 | 19.086 | −5.456 | 30.209 | 1.00 | 12.50 |
| ATOM | 1156 | CB | SER | A | 680 | 17.627 | −5.787 | 30.555 | 1.00 | 12.08 |
| ATOM | 1157 | OG | SER | A | 680 | 17.267 | −7.087 | 30.111 | 1.00 | 11.26 |
| ATOM | 1158 | C | SER | A | 680 | 19.409 | −4.038 | 30.663 | 1.00 | 11.78 |
| ATOM | 1159 | O | SER | A | 680 | 18.517 | −3.280 | 31.045 | 1.00 | 12.76 |
| ATOM | 1160 | N | VAL | A | 681 | 20.693 | −3.693 | 30.617 | 1.00 | 9.67 |
| ATOM | 1161 | CA | VAL | A | 681 | 21.183 | −2.386 | 31.049 | 1.00 | 7.19 |
| ATOM | 1162 | CB | VAL | A | 681 | 22.688 | −2.248 | 30.725 | 1.00 | 11.97 |
| ATOM | 1163 | CG1 | VAL | A | 681 | 23.219 | −0.917 | 31.245 | 1.00 | 10.98 |
| ATOM | 1164 | CG2 | VAL | A | 681 | 23.460 | −3.423 | 31.346 | 1.00 | 12.93 |
| ATOM | 1165 | C | VAL | A | 681 | 20.426 | −1.195 | 30.453 | 1.00 | 9.58 |
| ATOM | 1166 | O | VAL | A | 681 | 19.870 | −0.378 | 31.185 | 1.00 | 10.02 |
| ATOM | 1167 | N | LEU | A | 681A | 20.420 | −1.094 | 29.128 | 1.00 | 9.51 |
| ATOM | 1168 | CA | LEU | A | 681A | 19.718 | −0.011 | 28.443 | 1.00 | 10.16 |
| ATOM | 1169 | CB | LEU | A | 681A | 19.773 | −0.216 | 26.929 | 1.00 | 7.24 |
| ATOM | 1170 | CG | LEU | A | 681A | 21.079 | 0.035 | 26.178 | 1.00 | 9.74 |
| ATOM | 1171 | CD1 | LEU | A | 681A | 20.979 | −0.563 | 24.788 | 1.00 | 13.02 |
| ATOM | 1172 | CD2 | LEU | A | 681A | 21.345 | 1.526 | 26.102 | 1.00 | 15.84 |
| ATOM | 1173 | C | LEU | A | 681A | 18.253 | 0.049 | 28.851 | 1.00 | 8.34 |
| ATOM | 1174 | O | LEU | A | 681A | 17.725 | 1.118 | 29.174 | 1.00 | 10.62 |
| ATOM | 1175 | N | GLU | A | 682 | 17.599 | −1.110 | 28.834 | 1.00 | 10.08 |
| ATOM | 1176 | CA | GLU | A | 682 | 16.183 | −1.191 | 29.161 | 1.00 | 7.57 |
| ATOM | 1177 | CB | GLU | A | 682 | 15.667 | −2.599 | 28.855 | 1.00 | 6.60 |
| ATOM | 1178 | CG | GLU | A | 682 | 15.731 | −2.954 | 27.348 | 1.00 | 8.78 |
| ATOM | 1179 | CD | GLU | A | 682 | 17.151 | −3.199 | 26.835 | 1.00 | 12.12 |
| ATOM | 1180 | OE1 | GLU | A | 682 | 18.001 | −3.693 | 27.614 | 1.00 | 10.38 |
| ATOM | 1181 | OE2 | GLU | A | 682 | 17.418 | −2.919 | 25.646 | 1.00 | 10.27 |
| ATOM | 1182 | C | GLU | A | 682 | 15.867 | −0.773 | 30.594 | 1.00 | 8.81 |
| ATOM | 1183 | O | GLU | A | 682 | 14.829 | −0.158 | 30.854 | 1.00 | 7.94 |
| ATOM | 1184 | N | HIS | A | 683 | 16.752 | −1.099 | 31.526 | 1.00 | 9.05 |
| ATOM | 1185 | CA | HIS | A | 683 | 16.531 | −0.677 | 32.897 | 1.00 | 10.40 |
| ATOM | 1186 | CB | HIS | A | 683 | 17.561 | −1.324 | 33.830 | 1.00 | 11.92 |
| ATOM | 1187 | CG | HIS | A | 683 | 17.161 | −2.686 | 34.296 | 1.00 | 10.38 |
| ATOM | 1188 | CD2 | HIS | A | 683 | 17.557 | −3.920 | 33.903 | 1.00 | 9.93 |
| ATOM | 1189 | ND1 | HIS | A | 683 | 16.172 | −2.888 | 35.238 | 1.00 | 14.33 |
| ATOM | 1190 | CE1 | HIS | A | 683 | 15.977 | −4.183 | 35.400 | 1.00 | 7.54 |
| ATOM | 1191 | NE2 | HIS | A | 683 | 16.806 | −4.831 | 34.599 | 1.00 | 15.67 |
| ATOM | 1192 | C | HIS | A | 683 | 16.631 | 0.846 | 32.925 | 1.00 | 11.08 |
| ATOM | 1193 | O | HIS | A | 683 | 15.868 | 1.512 | 33.621 | 1.00 | 10.48 |
| ATOM | 1194 | N | HIS | A | 684 | 17.553 | 1.397 | 32.138 | 1.00 | 10.86 |
| ATOM | 1195 | CA | HIS | A | 684 | 17.710 | 2.844 | 32.076 | 1.00 | 9.98 |
| ATOM | 1196 | CB | HIS | A | 684 | 18.941 | 3.233 | 31.256 | 1.00 | 9.09 |
| ATOM | 1197 | CG | HIS | A | 684 | 19.229 | 4.697 | 31.284 | 1.00 | 8.54 |
| ATOM | 1198 | CD2 | HIS | A | 684 | 19.399 | 5.555 | 32.322 | 1.00 | 5.20 |
| ATOM | 1199 | ND1 | HIS | A | 684 | 19.313 | 5.467 | 30.142 | 1.00 | 17.15 |
| ATOM | 1200 | CE1 | HIS | A | 684 | 19.518 | 6.728 | 30.474 | 1.00 | 9.30 |
| ATOM | 1201 | NE2 | HIS | A | 684 | 19.573 | 6.807 | 31.795 | 1.00 | 15.79 |
| ATOM | 1202 | C | HIS | A | 684 | 16.473 | 3.502 | 31.465 | 1.00 | 9.44 |
| ATOM | 1203 | O | HIS | A | 684 | 16.020 | 4.542 | 31.941 | 1.00 | 11.75 |
| ATOM | 1204 | N | HIS | A | 685 | 15.929 | 2.898 | 30.411 | 1.00 | 9.42 |
| ATOM | 1205 | CA | HIS | A | 685 | 14.740 | 3.447 | 29.757 | 1.00 | 7.57 |
| ATOM | 1206 | CB | HIS | A | 685 | 14.361 | 2.621 | 28.521 | 1.00 | 14.86 |
| ATOM | 1207 | CG | HIS | A | 685 | 15.472 | 2.468 | 27.521 | 1.00 | 7.24 |
| ATOM | 1208 | CD2 | HIS | A | 685 | 15.846 | 1.402 | 26.773 | 1.00 | 9.32 |
| ATOM | 1209 | ND1 | HIS | A | 685 | 16.299 | 3.506 | 27.161 | 1.00 | 8.86 |
| ATOM | 1210 | CE1 | HIS | A | 685 | 17.141 | 3.088 | 26.227 | 1.00 | 14.03 |
| ATOM | 1211 | NE2 | HIS | A | 685 | 16.886 | 1.819 | 25.974 | 1.00 | 8.62 |
| ATOM | 1212 | C | HIS | A | 685 | 13.574 | 3.446 | 30.747 | 1.00 | 9.27 |
| ATOM | 1213 | O | HIS | A | 685 | 12.798 | 4.401 | 30.805 | 1.00 | 9.78 |
| ATOM | 1214 | N | PHE | A | 686 | 13.439 | 2.376 | 31.523 | 1.00 | 8.10 |
| ATOM | 1215 | CA | PHE | A | 686 | 12.351 | 2.314 | 32.507 | 1.00 | 10.05 |
| ATOM | 1216 | CB | PHE | A | 686 | 12.291 | 0.923 | 33.151 | 1.00 | 8.82 |
| ATOM | 1217 | CG | PHE | A | 686 | 11.281 | 0.808 | 34.263 | 1.00 | 10.55 |
| ATOM | 1218 | CD1 | PHE | A | 686 | 9.937 | 1.078 | 34.035 | 1.00 | 10.53 |
| ATOM | 1219 | CD2 | PHE | A | 686 | 11.674 | 0.442 | 35.538 | 1.00 | 11.42 |
| ATOM | 1220 | CE1 | PHE | A | 686 | 9.013 | 0.985 | 35.061 | 1.00 | 14.12 |
| ATOM | 1221 | CE2 | PHE | A | 686 | 10.754 | 0.348 | 36.568 | 1.00 | 15.20 |
| ATOM | 1222 | CZ | PHE | A | 686 | 9.423 | 0.618 | 36.328 | 1.00 | 13.66 |
| ATOM | 1223 | C | PHE | A | 686 | 12.554 | 3.391 | 33.570 | 1.00 | 9.59 |
| ATOM | 1224 | O | PHE | A | 686 | 11.599 | 4.036 | 34.022 | 1.00 | 11.10 |
| ATOM | 1225 | N | ASP | A | 687 | 13.807 | 3.603 | 33.965 | 1.00 | 10.38 |
| ATOM | 1226 | CA | ASP | A | 687 | 14.169 | 4.620 | 34.950 | 1.00 | 13.63 |
| ATOM | 1227 | CB | ASP | A | 687 | 15.699 | 4.646 | 35.147 | 1.00 | 15.63 |
| ATOM | 1228 | CG | ASP | A | 687 | 16.163 | 5.810 | 36.032 | 1.00 | 35.43 |
| ATOM | 1229 | OD1 | ASP | A | 687 | 15.842 | 5.812 | 37.238 | 1.00 | 39.21 |
| ATOM | 1230 | OD2 | ASP | A | 687 | 16.855 | 6.721 | 35.514 | 1.00 | 46.67 |
| ATOM | 1231 | C | ASP | A | 687 | 13.701 | 5.983 | 34.453 | 1.00 | 11.19 |
| ATOM | 1232 | O | ASP | A | 687 | 13.079 | 6.742 | 35.191 | 1.00 | 12.63 |
| ATOM | 1233 | N | GLN | A | 688 | 14.026 | 6.266 | 33.194 | 1.00 | 10.97 |
| ATOM | 1234 | CA | GLN | A | 688 | 13.664 | 7.531 | 32.571 | 1.00 | 10.87 |
| ATOM | 1235 | CB | GLN | A | 688 | 14.331 | 7.623 | 31.193 | 1.00 | 15.23 |
| ATOM | 1236 | CG | GLN | A | 688 | 15.844 | 7.876 | 31.260 | 1.00 | 11.96 |
| ATOM | 1237 | CD | GLN | A | 688 | 16.179 | 9.350 | 31.456 | 1.00 | 14.39 |
| ATOM | 1238 | OE1 | GLN | A | 688 | 15.805 | 10.193 | 30.640 | 1.00 | 20.66 |
| ATOM | 1239 | NE2 | GLN | A | 688 | 16.881 | 9.663 | 32.535 | 1.00 | 29.50 |
| ATOM | 1240 | C | GLN | A | 688 | 12.158 | 7.651 | 32.455 | 1.00 | 10.36 |
| ATOM | 1241 | O | GLN | A | 688 | 11.587 | 8.729 | 32.641 | 1.00 | 10.53 |
| ATOM | 1242 | N | CYS | A | 689 | 11.495 | 6.536 | 32.160 | 1.00 | 8.48 |
| ATOM | 1243 | CA | CYS | A | 689 | 10.043 | 6.517 | 32.040 | 1.00 | 9.59 |
| ATOM | 1244 | CB | CYS | A | 689 | 9.558 | 5.097 | 31.706 | 1.00 | 11.54 |
| ATOM | 1245 | SG | CYS | A | 689 | 7.772 | 4.940 | 31.573 | 1.00 | 11.98 |
| ATOM | 1246 | C | CYS | A | 689 | 9.406 | 7.005 | 33.343 | 1.00 | 10.54 |
| ATOM | 1247 | O | CYS | A | 689 | 8.559 | 7.905 | 33.331 | 1.00 | 10.86 |
| ATOM | 1248 | N | LEU | A | 690 | 9.822 | 6.417 | 34.463 | 1.00 | 12.43 |
| ATOM | 1249 | CA | LEU | A | 690 | 9.316 | 6.786 | 35.787 | 1.00 | 13.70 |
| ATOM | 1250 | CB | LEU | A | 690 | 9.968 | 5.912 | 36.863 | 1.00 | 15.35 |
| ATOM | 1251 | CG | LEU | A | 690 | 9.620 | 4.423 | 36.853 | 1.00 | 18.35 |
| ATOM | 1252 | CD1 | LEU | A | 690 | 10.326 | 3.723 | 38.008 | 1.00 | 25.25 |
| ATOM | 1253 | CD2 | LEU | A | 690 | 8.118 | 4.248 | 36.969 | 1.00 | 17.06 |
| ATOM | 1254 | C | LEU | A | 690 | 9.605 | 8.247 | 36.093 | 1.00 | 14.05 |
| ATOM | 1255 | O | LEU | A | 690 | 8.767 | 8.975 | 36.633 | 1.00 | 15.63 |
| ATOM | 1256 | N | MET | A | 691 | 10.817 | 8.681 | 35.750 | 1.00 | 13.87 |
| ATOM | 1257 | CA | MET | A | 691 | 11.256 | 10.042 | 35.975 | 1.00 | 14.41 |
| ATOM | 1258 | CB | MET | A | 691 | 12.676 | 10.212 | 35.411 | 1.00 | 14.62 |
| ATOM | 1259 | CG | MET | A | 691 | 13.371 | 11.496 | 35.757 | 1.00 | 32.36 |
| ATOM | 1260 | SD | MET | A | 691 | 12.902 | 12.755 | 34.635 | 1.00 | 45.55 |
| ATOM | 1261 | CE | MET | A | 691 | 13.918 | 12.471 | 33.200 | 1.00 | 37.72 |
| ATOM | 1262 | C | MET | A | 691 | 10.285 | 11.024 | 35.336 | 1.00 | 13.72 |
| ATOM | 1263 | O | MET | A | 691 | 9.863 | 11.990 | 35.971 | 1.00 | 14.45 |
| ATOM | 1264 | N | ILE | A | 692 | 9.903 | 10.758 | 34.091 | 1.00 | 10.48 |
| ATOM | 1265 | CA | ILE | A | 692 | 8.980 | 11.644 | 33.400 | 1.00 | 9.98 |
| ATOM | 1266 | CB | ILE | A | 692 | 8.966 | 11.371 | 31.895 | 1.00 | 10.00 |
| ATOM | 1267 | CG2 | ILE | A | 692 | 7.909 | 12.240 | 31.224 | 1.00 | 12.51 |
| ATOM | 1268 | CG1 | ILE | A | 692 | 10.346 | 11.691 | 31.315 | 1.00 | 14.40 |
| ATOM | 1269 | CD1 | ILE | A | 692 | 10.482 | 11.400 | 29.841 | 1.00 | 19.81 |
| ATOM | 1270 | C | ILE | A | 692 | 7.578 | 11.534 | 33.973 | 1.00 | 10.09 |
| ATOM | 1271 | O | ILE | A | 692 | 6.906 | 12.547 | 34.166 | 1.00 | 11.41 |
| ATOM | 1272 | N | LEU | A | 693 | 7.149 | 10.316 | 34.288 | 1.00 | 12.13 |
| ATOM | 1273 | CA | LEU | A | 693 | 5.821 | 10.111 | 34.862 | 1.00 | 13.85 |
| ATOM | 1274 | CB | LEU | A | 693 | 5.548 | 8.615 | 35.058 | 1.00 | 13.96 |
| ATOM | 1275 | CG | LEU | A | 693 | 5.212 | 7.826 | 33.789 | 1.00 | 10.33 |
| ATOM | 1276 | CD1 | LEU | A | 693 | 5.183 | 6.335 | 34.082 | 1.00 | 17.28 |
| ATOM | 1277 | CD2 | LEU | A | 693 | 3.868 | 8.305 | 33.243 | 1.00 | 9.40 |
| ATOM | 1278 | C | LEU | A | 693 | 5.644 | 10.837 | 36.193 | 1.00 | 14.19 |
| ATOM | 1279 | O | LEU | A | 693 | 4.524 | 11.179 | 36.577 | 1.00 | 16.64 |
| ATOM | 1280 | N | ASN | A | 694 | 6.747 | 11.073 | 36.893 | 1.00 | 16.46 |
| ATOM | 1281 | CA | ASN | A | 694 | 6.697 | 11.755 | 38.183 | 1.00 | 16.63 |
| ATOM | 1282 | CB | ASN | A | 694 | 7.686 | 11.116 | 39.159 | 1.00 | 20.12 |
| ATOM | 1283 | CG | ASN | A | 694 | 7.231 | 9.760 | 39.641 | 1.00 | 24.99 |
| ATOM | 1284 | OD1 | ASN | A | 694 | 7.869 | 8.742 | 39.373 | 1.00 | 30.33 |
| ATOM | 1285 | ND2 | ASN | A | 694 | 6.115 | 9.736 | 40.360 | 1.00 | 25.93 |
| ATOM | 1286 | C | ASN | A | 694 | 6.989 | 13.247 | 38.101 | 1.00 | 18.31 |
| ATOM | 1287 | O | ASN | A | 694 | 6.855 | 13.958 | 39.093 | 1.00 | 18.26 |
| ATOM | 1288 | N | SER | A | 695 | 7.395 | 13.721 | 36.930 | 1.00 | 16.05 |
| ATOM | 1289 | CA | SER | A | 695 | 7.704 | 15.139 | 36.755 | 1.00 | 17.70 |
| ATOM | 1290 | CB | SER | A | 695 | 8.321 | 15.376 | 35.376 | 1.00 | 19.30 |
| ATOM | 1291 | OG | SER | A | 695 | 9.607 | 14.793 | 35.298 | 1.00 | 34.04 |
| ATOM | 1292 | C | SER | A | 695 | 6.471 | 16.022 | 36.914 | 1.00 | 15.83 |
| ATOM | 1293 | O | SER | A | 695 | 5.400 | 15.710 | 36.406 | 1.00 | 17.46 |
| ATOM | 1294 | N | PRO | A | 696 | 6.611 | 17.144 | 37.630 | 1.00 | 18.97 |
| ATOM | 1295 | CD | PRO | A | 696 | 7.779 | 17.577 | 38.415 | 1.00 | 26.97 |
| ATOM | 1296 | CA | PRO | A | 696 | 5.480 | 18.053 | 37.830 | 1.00 | 18.99 |
| ATOM | 1297 | CB | PRO | A | 696 | 6.113 | 19.220 | 38.579 | 1.00 | 22.14 |
| ATOM | 1298 | CG | PRO | A | 696 | 7.154 | 18.534 | 39.414 | 1.00 | 28.71 |
| ATOM | 1299 | C | PRO | A | 696 | 4.866 | 18.480 | 36.498 | 1.00 | 16.02 |
| ATOM | 1300 | O | PRO | A | 696 | 5.585 | 18.839 | 35.564 | 1.00 | 18.08 |
| ATOM | 1301 | N | GLY | A | 697 | 3.539 | 18.425 | 36.418 | 1.00 | 16.17 |
| ATOM | 1302 | CA | GLY | A | 697 | 2.843 | 18.804 | 35.201 | 1.00 | 16.65 |
| ATOM | 1303 | C | GLY | A | 697 | 2.862 | 17.725 | 34.130 | 1.00 | 14.72 |
| ATOM | 1304 | O | GLY | A | 697 | 2.283 | 17.901 | 33.060 | 1.00 | 15.86 |
| ATOM | 1305 | N | ASN | A | 698 | 3.511 | 16.601 | 34.424 | 1.00 | 14.68 |
| ATOM | 1306 | CA | ASN | A | 698 | 3.621 | 15.493 | 33.471 | 1.00 | 14.66 |
| ATOM | 1307 | CB | ASN | A | 698 | 5.099 | 15.209 | 33.174 | 1.00 | 15.25 |
| ATOM | 1308 | CG | ASN | A | 698 | 5.760 | 16.304 | 32.365 | 1.00 | 19.94 |
| ATOM | 1309 | OD1 | ASN | A | 698 | 5.883 | 16.199 | 31.146 | 1.00 | 13.89 |
| ATOM | 1310 | ND2 | ASN | A | 698 | 6.186 | 17.367 | 33.039 | 1.00 | 16.33 |
| ATOM | 1311 | C | ASN | A | 698 | 2.990 | 14.198 | 33.973 | 1.00 | 14.82 |
| ATOM | 1312 | O | ASN | A | 698 | 3.044 | 13.174 | 33.293 | 1.00 | 13.20 |
| ATOM | 1313 | N | GLN | A | 699 | 2.379 | 14.243 | 35.151 | 1.00 | 13.46 |
| ATOM | 1314 | CA | GLN | A | 699 | 1.807 | 13.041 | 35.749 | 1.00 | 14.53 |
| ATOM | 1315 | CB | GLN | A | 699 | 1.652 | 13.281 | 37.251 | 1.00 | 16.25 |
| ATOM | 1316 | CG | GLN | A | 699 | 2.957 | 13.713 | 37.902 | 1.00 | 17.08 |
| ATOM | 1317 | CD | GLN | A | 699 | 2.787 | 14.103 | 39.350 | 1.00 | 37.15 |
| ATOM | 1318 | OE1 | GLN | A | 699 | 2.147 | 15.105 | 39.661 | 1.00 | 41.13 |
| ATOM | 1319 | NE2 | GLN | A | 699 | 3.357 | 13.307 | 40.248 | 1.00 | 41.91 |
| ATOM | 1320 | C | GLN | A | 699 | 0.499 | 12.522 | 35.158 | 1.00 | 13.96 |
| ATOM | 1321 | O | GLN | A | 699 | −0.567 | 12.676 | 35.754 | 1.00 | 15.67 |
| ATOM | 1322 | N | ILE | A | 700 | 0.582 | 11.867 | 34.003 | 1.00 | 13.44 |
| ATOM | 1323 | CA | ILE | A | 700 | −0.617 | 11.348 | 33.356 | 1.00 | 14.25 |
| ATOM | 1324 | CB | ILE | A | 700 | −0.345 | 10.974 | 31.881 | 1.00 | 15.57 |
| ATOM | 1325 | CG2 | ILE | A | 700 | 0.010 | 12.222 | 31.086 | 1.00 | 15.78 |
| ATOM | 1326 | CG1 | ILE | A | 700 | 0.777 | 9.941 | 31.793 | 1.00 | 16.74 |
| ATOM | 1327 | CD1 | ILE | A | 700 | 0.940 | 9.361 | 30.407 | 1.00 | 12.98 |
| ATOM | 1328 | C | ILE | A | 700 | −1.262 | 10.151 | 34.064 | 1.00 | 13.88 |
| ATOM | 1329 | O | ILE | A | 700 | −2.342 | 9.709 | 33.673 | 1.00 | 14.53 |
| ATOM | 1330 | N | LEU | A | 701 | −0.609 | 9.624 | 35.098 | 1.00 | 11.60 |
| ATOM | 1331 | CA | LEU | A | 701 | −1.169 | 8.495 | 35.845 | 1.00 | 14.58 |
| ATOM | 1332 | CB | LEU | A | 701 | −0.150 | 7.358 | 35.965 | 1.00 | 13.85 |
| ATOM | 1333 | CG | LEU | A | 701 | 0.310 | 6.673 | 34.677 | 1.00 | 11.07 |
| ATOM | 1334 | CD1 | LEU | A | 701 | 1.270 | 5.539 | 35.031 | 1.00 | 13.34 |
| ATOM | 1335 | CD2 | LEU | A | 701 | −0.895 | 6.128 | 33.921 | 1.00 | 12.89 |
| ATOM | 1336 | C | LEU | A | 701 | −1.612 | 8.914 | 37.247 | 1.00 | 13.75 |
| ATOM | 1337 | O | LEU | A | 701 | −2.053 | 8.084 | 38.040 | 1.00 | 15.74 |
| ATOM | 1338 | N | SER | A | 702 | −1.497 | 10.201 | 37.547 | 1.00 | 15.74 |
| ATOM | 1339 | CA | SER | A | 702 | −1.872 | 10.717 | 38.860 | 1.00 | 18.36 |
| ATOM | 1340 | CB | SER | A | 702 | −1.573 | 12.217 | 38.949 | 1.00 | 20.19 |
| ATOM | 1341 | OG | SER | A | 702 | −2.446 | 12.958 | 38.113 | 1.00 | 21.94 |
| ATOM | 1342 | C | SER | A | 702 | −3.347 | 10.490 | 39.176 | 1.00 | 19.34 |
| ATOM | 1343 | O | SER | A | 702 | −3.754 | 10.559 | 40.336 | 1.00 | 20.75 |
| ATOM | 1344 | N | GLY | A | 703 | −4.144 | 10.224 | 38.148 | 1.00 | 18.92 |
| ATOM | 1345 | CA | GLY | A | 703 | −5.565 | 10.012 | 38.359 | 1.00 | 20.40 |
| ATOM | 1346 | C | GLY | A | 703 | −5.944 | 8.600 | 38.764 | 1.00 | 21.19 |
| ATOM | 1347 | O | GLY | A | 703 | −7.036 | 8.369 | 39.284 | 1.00 | 20.43 |
| ATOM | 1348 | N | LEU | A | 704 | −5.045 | 7.649 | 38.539 | 1.00 | 16.00 |
| ATOM | 1349 | CA | LEU | A | 704 | −5.326 | 6.259 | 38.876 | 1.00 | 15.59 |
| ATOM | 1350 | CB | LEU | A | 704 | −4.262 | 5.335 | 38.278 | 1.00 | 9.29 |
| ATOM | 1351 | CG | LEU | A | 704 | −4.459 | 4.921 | 36.823 | 1.00 | 14.10 |
| ATOM | 1352 | CD1 | LEU | A | 704 | −4.510 | 6.143 | 35.926 | 1.00 | 17.73 |
| ATOM | 1353 | CD2 | LEU | A | 704 | −3.312 | 3.998 | 36.418 | 1.00 | 11.19 |
| ATOM | 1354 | C | LEU | A | 704 | −5.419 | 5.993 | 40.368 | 1.00 | 16.40 |
| ATOM | 1355 | O | LEU | A | 704 | −4.773 | 6.659 | 41.178 | 1.00 | 16.88 |
| ATOM | 1356 | N | SER | A | 705 | −6.233 | 5.004 | 40.721 | 1.00 | 15.43 |
| ATOM | 1357 | CA | SER | A | 705 | −6.395 | 4.621 | 42.111 | 1.00 | 15.96 |
| ATOM | 1358 | CB | SER | A | 705 | −7.556 | 3.640 | 42.268 | 1.00 | 15.57 |
| ATOM | 1359 | OG | SER | A | 705 | −7.242 | 2.402 | 41.658 | 1.00 | 15.44 |
| ATOM | 1360 | C | SER | A | 705 | −5.092 | 3.947 | 42.515 | 1.00 | 16.32 |
| ATOM | 1361 | O | SER | A | 705 | −4.280 | 3.583 | 41.662 | 1.00 | 17.25 |
| ATOM | 1362 | N | ILE | A | 706 | −4.886 | 3.782 | 43.812 | 1.00 | 17.56 |
| ATOM | 1363 | CA | ILE | A | 706 | −3.666 | 3.164 | 44.292 | 1.00 | 16.88 |
| ATOM | 1364 | CB | ILE | A | 706 | −3.679 | 3.086 | 45.836 | 1.00 | 21.33 |
| ATOM | 1365 | CG2 | ILE | A | 706 | −2.466 | 2.331 | 46.335 | 1.00 | 23.53 |
| ATOM | 1366 | CG1 | ILE | A | 706 | −3.712 | 4.507 | 46.411 | 1.00 | 21.96 |
| ATOM | 1367 | CD1 | ILE | A | 706 | −3.892 | 4.577 | 47.918 | 1.00 | 22.24 |
| ATOM | 1368 | C | ILE | A | 706 | −3.415 | 1.782 | 43.685 | 1.00 | 16.48 |
| ATOM | 1369 | O | ILE | A | 706 | −2.291 | 1.470 | 43.291 | 1.00 | 18.62 |
| ATOM | 1370 | N | GLU | A | 707 | −4.456 | 0.962 | 43.584 | 1.00 | 15.92 |
| ATOM | 1371 | CA | GLU | A | 707 | −4.304 | −0.376 | 43.019 | 1.00 | 14.53 |
| ATOM | 1372 | CB | GLU | A | 707 | −5.509 | −1.250 | 43.378 | 1.00 | 19.33 |
| ATOM | 1373 | CG | GLU | A | 707 | −5.518 | −1.682 | 44.836 | 1.00 | 24.82 |
| ATOM | 1374 | CD | GLU | A | 707 | −4.269 | −2.460 | 45.214 | 1.00 | 31.26 |
| ATOM | 1375 | OE1 | GLU | A | 707 | −4.029 | −3.525 | 44.610 | 1.00 | 34.47 |
| ATOM | 1376 | OE2 | GLU | A | 707 | −3.525 | −2.006 | 46.110 | 1.00 | 30.79 |
| ATOM | 1377 | C | GLU | A | 707 | −4.093 | −0.385 | 41.507 | 1.00 | 14.27 |
| ATOM | 1378 | O | GLU | A | 707 | −3.283 | −1.156 | 41.002 | 1.00 | 15.02 |
| ATOM | 1379 | N | GLU | A | 708 | −4.818 | 0.458 | 40.782 | 1.00 | 11.81 |
| ATOM | 1380 | CA | GLU | A | 708 | −4.644 | 0.495 | 39.337 | 1.00 | 12.49 |
| ATOM | 1381 | CB | GLU | A | 708 | −5.756 | 1.317 | 38.673 | 1.00 | 14.73 |
| ATOM | 1382 | CG | GLU | A | 708 | −7.134 | 0.663 | 38.813 | 1.00 | 11.98 |
| ATOM | 1383 | CD | GLU | A | 708 | −8.088 | 1.020 | 37.683 | 1.00 | 18.01 |
| ATOM | 1384 | OE1 | GLU | A | 708 | −8.121 | 2.196 | 37.274 | 1.00 | 19.76 |
| ATOM | 1385 | OE2 | GLU | A | 708 | −8.816 | 0.120 | 37.216 | 1.00 | 22.22 |
| ATOM | 1386 | C | GLU | A | 708 | −3.264 | 1.054 | 38.996 | 1.00 | 14.00 |
| ATOM | 1387 | O | GLU | A | 708 | −2.624 | 0.607 | 38.047 | 1.00 | 12.79 |
| ATOM | 1388 | N | TYR | A | 709 | −2.798 | 2.018 | 39.784 | 1.00 | 12.70 |
| ATOM | 1389 | CA | TYR | A | 709 | −1.482 | 2.604 | 39.551 | 1.00 | 13.42 |
| ATOM | 1390 | CB | TYR | A | 709 | −1.204 | 3.724 | 40.557 | 1.00 | 12.05 |
| ATOM | 1391 | CG | TYR | A | 709 | 0.129 | 4.407 | 40.339 | 1.00 | 19.13 |
| ATOM | 1392 | CD1 | TYR | A | 709 | 0.311 | 5.302 | 39.296 | 1.00 | 14.81 |
| ATOM | 1393 | CE1 | TYR | A | 709 | 1.530 | 5.928 | 39.091 | 1.00 | 15.81 |
| ATOM | 1394 | CD2 | TYR | A | 709 | 1.207 | 4.149 | 41.174 | 1.00 | 17.39 |
| ATOM | 1395 | CE2 | TYR | A | 709 | 2.427 | 4.767 | 40.978 | 1.00 | 18.97 |
| ATOM | 1396 | CZ | TYR | A | 709 | 2.583 | 5.658 | 39.934 | 1.00 | 17.67 |
| ATOM | 1397 | OH | TYR | A | 709 | 3.793 | 6.284 | 39.737 | 1.00 | 18.77 |
| ATOM | 1398 | C | TYR | A | 709 | −0.424 | 1.515 | 39.708 | 1.00 | 15.44 |
| ATOM | 1399 | O | TYR | A | 709 | 0.452 | 1.348 | 38.855 | 1.00 | 15.06 |
| ATOM | 1400 | N | LYS | A | 710 | −0.520 | 0.774 | 40.808 | 1.00 | 15.25 |
| ATOM | 1401 | CA | LYS | A | 710 | 0.409 | −0.311 | 41.095 | 1.00 | 15.31 |
| ATOM | 1402 | CB | LYS | A | 710 | −0.020 | −1.026 | 42.379 | 1.00 | 22.10 |
| ATOM | 1403 | CG | LYS | A | 710 | 0.809 | −2.249 | 42.716 | 1.00 | 23.74 |
| ATOM | 1404 | CD | LYS | A | 710 | 0.299 | −2.931 | 43.978 | 1.00 | 33.24 |
| ATOM | 1405 | CE | LYS | A | 710 | 0.996 | −4.263 | 44.204 | 1.00 | 39.08 |
| ATOM | 1406 | NZ | LYS | A | 710 | 2.478 | −4.117 | 44.248 | 1.00 | 42.37 |
| ATOM | 1407 | C | LYS | A | 710 | 0.474 | −1.309 | 39.939 | 1.00 | 14.94 |
| ATOM | 1408 | O | LYS | A | 710 | 1.555 | −1.645 | 39.448 | 1.00 | 12.87 |
| ATOM | 1409 | N | THR | A | 711 | −0.692 | −1.777 | 39.506 | 1.00 | 13.50 |
| ATOM | 1410 | CA | THR | A | 711 | −0.783 | −2.736 | 38.411 | 1.00 | 12.45 |
| ATOM | 1411 | CB | THR | A | 711 | −2.249 | −3.092 | 38.128 | 1.00 | 17.44 |
| ATOM | 1412 | OG1 | THR | A | 711 | −2.791 | −3.792 | 39.255 | 1.00 | 21.96 |
| ATOM | 1413 | CG2 | THR | A | 711 | −2.365 | −3.953 | 36.872 | 1.00 | 16.75 |
| ATOM | 1414 | C | THR | A | 711 | −0.161 | −2.198 | 37.129 | 1.00 | 11.72 |
| ATOM | 1415 | O | THR | A | 711 | 0.597 | −2.896 | 36.442 | 1.00 | 13.19 |
| ATOM | 1416 | N | THR | A | 712 | −0.487 | −0.954 | 36.809 | 1.00 | 11.15 |
| ATOM | 1417 | CA | THR | A | 712 | 0.015 | −0.319 | 35.599 | 1.00 | 13.99 |
| ATOM | 1418 | CB | THR | A | 712 | −0.598 | 1.081 | 35.424 | 1.00 | 9.97 |
| ATOM | 1419 | OG1 | THR | A | 712 | −2.029 | 0.967 | 35.357 | 1.00 | 14.40 |
| ATOM | 1420 | CG2 | THR | A | 712 | −0.088 | 1.729 | 34.133 | 1.00 | 12.92 |
| ATOM | 1421 | C | THR | A | 712 | 1.541 | −0.221 | 35.556 | 1.00 | 12.77 |
| ATOM | 1422 | O | THR | A | 712 | 2.158 | −0.596 | 34.559 | 1.00 | 12.54 |
| ATOM | 1423 | N | LEU | A | 713 | 2.156 | 0.275 | 36.625 | 1.00 | 12.50 |
| ATOM | 1424 | CA | LEU | A | 713 | 3.612 | 0.384 | 36.638 | 1.00 | 11.02 |
| ATOM | 1425 | CB | LEU | A | 713 | 4.105 | 1.023 | 37.940 | 1.00 | 13.58 |
| ATOM | 1426 | CG | LEU | A | 713 | 3.889 | 2.528 | 38.100 | 1.00 | 22.03 |
| ATOM | 1427 | CD1 | LEU | A | 713 | 4.754 | 3.042 | 39.250 | 1.00 | 21.64 |
| ATOM | 1428 | CD2 | LEU | A | 713 | 4.261 | 3.241 | 36.809 | 1.00 | 27.88 |
| ATOM | 1429 | C | LEU | A | 713 | 4.298 | −0.967 | 36.438 | 1.00 | 13.04 |
| ATOM | 1430 | O | LEU | A | 713 | 5.340 | −1.048 | 35.785 | 1.00 | 12.26 |
| ATOM | 1431 | N | LYS | A | 714 | 3.718 | −2.026 | 36.995 | 1.00 | 11.66 |
| ATOM | 1432 | CA | LYS | A | 714 | 4.304 | −3.356 | 36.852 | 1.00 | 12.24 |
| ATOM | 1433 | CB | LYS | A | 714 | 3.540 | −4.383 | 37.690 | 1.00 | 17.46 |
| ATOM | 1434 | CG | LYS | A | 714 | 3.582 | −4.123 | 39.182 | 1.00 | 27.51 |
| ATOM | 1435 | CD | LYS | A | 714 | 2.878 | −5.236 | 39.939 | 1.00 | 33.99 |
| ATOM | 1436 | CE | LYS | A | 714 | 2.899 | −4.988 | 41.433 | 1.00 | 42.93 |
| ATOM | 1437 | NZ | LYS | A | 714 | 2.222 | −6.092 | 42.168 | 1.00 | 46.33 |
| ATOM | 1438 | C | LYS | A | 714 | 4.284 | −3.784 | 35.390 | 1.00 | 12.70 |
| ATOM | 1439 | O | LYS | A | 714 | 5.259 | −4.338 | 34.886 | 1.00 | 12.91 |
| ATOM | 1440 | N | ILE | A | 715 | 3.168 | −3.525 | 34.717 | 1.00 | 10.95 |
| ATOM | 1441 | CA | ILE | A | 715 | 3.023 | −3.869 | 33.308 | 1.00 | 10.57 |
| ATOM | 1442 | CB | ILE | A | 715 | 1.586 | −3.590 | 32.820 | 1.00 | 11.39 |
| ATOM | 1443 | CG2 | ILE | A | 715 | 1.468 | −3.896 | 31.339 | 1.00 | 11.35 |
| ATOM | 1444 | CG1 | ILE | A | 715 | 0.596 | −4.450 | 33.613 | 1.00 | 12.69 |
| ATOM | 1445 | CD1 | ILE | A | 715 | −0.854 | −4.130 | 33.332 | 1.00 | 13.44 |
| ATOM | 1446 | C | ILE | A | 715 | 4.017 | −3.052 | 32.482 | 1.00 | 10.34 |
| ATOM | 1447 | O | ILE | A | 715 | 4.676 | −3.581 | 31.588 | 1.00 | 11.86 |
| ATOM | 1448 | N | ILE | A | 716 | 4.133 | −1.762 | 32.793 | 1.00 | 10.78 |
| ATOM | 1449 | CA | ILE | A | 716 | 5.069 | −0.886 | 32.081 | 1.00 | 9.90 |
| ATOM | 1450 | CB | ILE | A | 716 | 4.989 | 0.563 | 32.621 | 1.00 | 10.08 |
| ATOM | 1451 | CG2 | ILE | A | 716 | 6.171 | 1.387 | 32.115 | 1.00 | 10.86 |
| ATOM | 1452 | CG1 | ILE | A | 716 | 3.661 | 1.197 | 32.189 | 1.00 | 10.91 |
| ATOM | 1453 | CD1 | ILE | A | 716 | 3.413 | 2.564 | 32.792 | 1.00 | 11.27 |
| ATOM | 1454 | C | ILE | A | 716 | 6.508 | −1.393 | 32.196 | 1.00 | 8.77 |
| ATOM | 1455 | O | ILE | A | 716 | 7.229 | −1.487 | 31.199 | 1.00 | 9.34 |
| ATOM | 1456 | N | LYS | A | 717 | 6.926 | −1.724 | 33.413 | 1.00 | 9.18 |
| ATOM | 1457 | CA | LYS | A | 717 | 8.275 | −2.235 | 33.623 | 1.00 | 10.88 |
| ATOM | 1458 | CB | LYS | A | 717 | 8.489 | −2.606 | 35.087 | 1.00 | 12.47 |
| ATOM | 1459 | CG | LYS | A | 717 | 9.790 | −3.366 | 35.337 | 1.00 | 14.78 |
| ATOM | 1460 | CD | LYS | A | 717 | 9.865 | −3.864 | 36.775 | 1.00 | 17.70 |
| ATOM | 1461 | CE | LYS | A | 717 | 11.045 | −4.809 | 36.977 | 1.00 | 29.85 |
| ATOM | 1462 | NZ | LYS | A | 717 | 11.082 | −5.374 | 38.355 | 1.00 | 25.71 |
| ATOM | 1463 | C | LYS | A | 717 | 8.540 | −3.469 | 32.752 | 1.00 | 10.54 |
| ATOM | 1464 | O | LYS | A | 717 | 9.536 | −3.524 | 32.041 | 1.00 | 10.05 |
| ATOM | 1465 | N | GLN | A | 718 | 7.647 | −4.451 | 32.847 | 1.00 | 11.40 |
| ATOM | 1466 | CA | GLN | A | 718 | 7.774 | −5.682 | 32.067 | 1.00 | 11.80 |
| ATOM | 1467 | CB | GLN | A | 718 | 6.635 | −6.628 | 32.434 | 1.00 | 11.33 |
| ATOM | 1468 | CG | GLN | A | 718 | 6.683 | −6.965 | 33.899 | 1.00 | 14.75 |
| ATOM | 1469 | CD | GLN | A | 718 | 7.954 | −7.691 | 34.293 | 1.00 | 35.80 |
| ATOM | 1470 | OE1 | GLN | A | 718 | 8.774 | −7.176 | 35.053 | 1.00 | 49.66 |
| ATOM | 1471 | NE2 | GLN | A | 718 | 8.115 | −8.909 | 33.784 | 1.00 | 43.62 |
| ATOM | 1472 | C | GLN | A | 718 | 7.778 | −5.422 | 30.559 | 1.00 | 9.82 |
| ATOM | 1473 | O | GLN | A | 718 | 8.559 | −6.027 | 29.831 | 1.00 | 9.86 |
| ATOM | 1474 | N | ALA | A | 719 | 6.920 | −4.518 | 30.108 | 1.00 | 8.83 |
| ATOM | 1475 | CA | ALA | A | 719 | 6.842 | −4.205 | 28.686 | 1.00 | 10.49 |
| ATOM | 1476 | CB | ALA | A | 719 | 5.657 | −3.292 | 28.436 | 1.00 | 5.96 |
| ATOM | 1477 | C | ALA | A | 719 | 8.129 | −3.560 | 28.186 | 1.00 | 9.98 |
| ATOM | 1478 | O | ALA | A | 719 | 8.618 | −3.892 | 27.101 | 1.00 | 9.19 |
| ATOM | 1479 | N | ILE | A | 720 | 8.688 | −2.641 | 28.971 | 1.00 | 7.25 |
| ATOM | 1480 | CA | ILE | A | 720 | 9.920 | −1.988 | 28.548 | 1.00 | 8.66 |
| ATOM | 1481 | CB | ILE | A | 720 | 10.233 | −0.753 | 29.426 | 1.00 | 10.86 |
| ATOM | 1482 | CG2 | ILE | A | 720 | 11.616 | −0.193 | 29.086 | 1.00 | 11.64 |
| ATOM | 1483 | CG1 | ILE | A | 720 | 9.164 | 0.320 | 29.181 | 1.00 | 11.59 |
| ATOM | 1484 | CD1 | ILE | A | 720 | 9.367 | 1.605 | 29.976 | 1.00 | 12.21 |
| ATOM | 1485 | C | ILE | A | 720 | 11.085 | −2.974 | 28.580 | 1.00 | 10.02 |
| ATOM | 1486 | O | ILE | A | 720 | 11.883 | −3.040 | 27.641 | 1.00 | 8.95 |
| ATOM | 1487 | N | LEU | A | 721 | 11.178 | −3.762 | 29.643 | 1.00 | 8.10 |
| ATOM | 1488 | CA | LEU | A | 721 | 12.264 | −4.733 | 29.719 | 1.00 | 8.45 |
| ATOM | 1489 | CB | LEU | A | 721 | 12.251 | −5.446 | 31.077 | 1.00 | 10.55 |
| ATOM | 1490 | CG | LEU | A | 721 | 12.613 | −4.553 | 32.267 | 1.00 | 13.26 |
| ATOM | 1491 | CD1 | LEU | A | 721 | 12.539 | −5.350 | 33.574 | 1.00 | 15.78 |
| ATOM | 1492 | CD2 | LEU | A | 721 | 14.006 | −3.991 | 32.059 | 1.00 | 24.08 |
| ATOM | 1493 | C | LEU | A | 721 | 12.173 | −5.750 | 28.576 | 1.00 | 9.96 |
| ATOM | 1494 | O | LEU | A | 721 | 13.195 | −6.196 | 28.049 | 1.00 | 8.91 |
| ATOM | 1495 | N | ALA | A | 722 | 10.948 | −6.082 | 28.175 | 1.00 | 8.77 |
| ATOM | 1496 | CA | ALA | A | 722 | 10.715 | −7.047 | 27.100 | 1.00 | 8.74 |
| ATOM | 1497 | CB | ALA | A | 722 | 9.219 | −7.218 | 26.879 | 1.00 | 9.06 |
| ATOM | 1498 | C | ALA | A | 722 | 11.383 | −6.648 | 25.786 | 1.00 | 10.10 |
| ATOM | 1499 | O | ALA | A | 722 | 11.704 | −7.507 | 24.965 | 1.00 | 13.26 |
| ATOM | 1500 | N | THR | A | 723 | 11.598 | −5.350 | 25.585 | 1.00 | 8.97 |
| ATOM | 1501 | CA | THR | A | 723 | 12.213 | −4.884 | 24.344 | 1.00 | 10.94 |
| ATOM | 1502 | CB | THR | A | 723 | 11.943 | −3.374 | 24.127 | 1.00 | 8.53 |
| ATOM | 1503 | OG1 | THR | A | 723 | 12.542 | −2.615 | 25.181 | 1.00 | 8.41 |
| ATOM | 1504 | CG2 | THR | A | 723 | 10.437 | −3.114 | 24.118 | 1.00 | 8.22 |
| ATOM | 1505 | C | THR | A | 723 | 13.713 | −5.187 | 24.203 | 1.00 | 9.21 |
| ATOM | 1506 | O | THR | A | 723 | 14.327 | −4.862 | 23.186 | 1.00 | 12.03 |
| ATOM | 1507 | N | ASP | A | 724 | 14.308 | −5.797 | 25.223 | 1.00 | 9.75 |
| ATOM | 1508 | CA | ASP | A | 724 | 15.710 | −6.203 | 25.133 | 1.00 | 7.82 |
| ATOM | 1509 | CB | ASP | A | 724 | 16.257 | −6.533 | 26.525 | 1.00 | 12.18 |
| ATOM | 1510 | CG | ASP | A | 724 | 17.648 | −7.147 | 26.490 | 1.00 | 8.16 |
| ATOM | 1511 | OD1 | ASP | A | 724 | 18.232 | −7.299 | 25.394 | 1.00 | 11.70 |
| ATOM | 1512 | OD2 | ASP | A | 724 | 18.161 | −7.481 | 27.581 | 1.00 | 11.61 |
| ATOM | 1513 | C | ASP | A | 724 | 15.600 | −7.475 | 24.293 | 1.00 | 8.26 |
| ATOM | 1514 | O | ASP | A | 724 | 15.022 | −8.459 | 24.742 | 1.00 | 10.28 |
| ATOM | 1515 | N | LEU | A | 725 | 16.127 | −7.452 | 23.073 | 1.00 | 9.50 |
| ATOM | 1516 | CA | LEU | A | 725 | 16.037 | −8.611 | 22.189 | 1.00 | 8.83 |
| ATOM | 1517 | CB | LEU | A | 725 | 16.791 | −8.343 | 20.883 | 1.00 | 11.33 |
| ATOM | 1518 | CG | LEU | A | 725 | 16.365 | −9.236 | 19.717 | 1.00 | 16.17 |
| ATOM | 1519 | CD1 | LEU | A | 725 | 14.910 | −8.953 | 19.367 | 1.00 | 12.02 |
| ATOM | 1520 | CD2 | LEU | A | 725 | 17.262 | −8.974 | 18.516 | 1.00 | 20.09 |
| ATOM | 1521 | C | LEU | A | 725 | 16.573 | −9.880 | 22.849 | 1.00 | 12.30 |
| ATOM | 1522 | O | LEU | A | 725 | 16.179 | −10.994 | 22.498 | 1.00 | 11.52 |
| ATOM | 1523 | N | ALA | A | 726 | 17.476 | −9.712 | 23.806 | 1.00 | 11.08 |
| ATOM | 1524 | CA | ALA | A | 726 | 18.028 | −10.855 | 24.517 | 1.00 | 12.63 |
| ATOM | 1525 | CB | ALA | A | 726 | 19.104 | −10.394 | 25.489 | 1.00 | 21.41 |
| ATOM | 1526 | C | ALA | A | 726 | 16.918 | −11.583 | 25.274 | 1.00 | 12.40 |
| ATOM | 1527 | O | ALA | A | 726 | 16.914 | −12.813 | 25.354 | 1.00 | 13.56 |
| ATOM | 1528 | N | LEU | A | 727 | 15.985 | −10.823 | 25.843 | 1.00 | 12.79 |
| ATOM | 1529 | CA | LEU | A | 727 | 14.884 | −11.421 | 26.591 | 1.00 | 14.99 |
| ATOM | 1530 | CB | LEU | A | 727 | 14.206 | −10.380 | 27.485 | 1.00 | 11.71 |
| ATOM | 1531 | CG | LEU | A | 727 | 15.096 | −9.856 | 28.612 | 1.00 | 16.19 |
| ATOM | 1532 | CD1 | LEU | A | 727 | 14.302 | −8.925 | 29.511 | 1.00 | 13.74 |
| ATOM | 1533 | CD2 | LEU | A | 727 | 15.631 | −11.036 | 29.409 | 1.00 | 19.43 |
| ATOM | 1534 | C | LEU | A | 727 | 13.868 | −12.045 | 25.646 | 1.00 | 15.02 |
| ATOM | 1535 | O | LEU | A | 727 | 13.173 | −12.996 | 26.005 | 1.00 | 15.57 |
| ATOM | 1536 | N | TYR | A | 728 | 13.777 | −11.500 | 24.438 | 1.00 | 12.38 |
| ATOM | 1537 | CA | TYR | A | 728 | 12.862 | −12.038 | 23.435 | 1.00 | 12.71 |
| ATOM | 1538 | CB | TYR | A | 728 | 12.844 | −11.136 | 22.194 | 1.00 | 10.64 |
| ATOM | 1539 | CG | TYR | A | 728 | 12.303 | −11.823 | 20.960 | 1.00 | 12.57 |
| ATOM | 1540 | CD1 | TYR | A | 728 | 10.966 | −12.197 | 20.875 | 1.00 | 14.71 |
| ATOM | 1541 | CE1 | TYR | A | 728 | 10.473 | −12.854 | 19.752 | 1.00 | 12.42 |
| ATOM | 1542 | CD2 | TYR | A | 728 | 13.139 | −12.124 | 19.888 | 1.00 | 13.44 |
| ATOM | 1543 | CE2 | TYR | A | 728 | 12.659 | −12.777 | 18.763 | 1.00 | 13.13 |
| ATOM | 1544 | CZ | TYR | A | 728 | 11.329 | −13.142 | 18.699 | 1.00 | 15.27 |
| ATOM | 1545 | OH | TYR | A | 728 | 10.857 | −13.801 | 17.580 | 1.00 | 18.62 |
| ATOM | 1546 | C | TYR | A | 728 | 13.350 | −13.430 | 23.041 | 1.00 | 12.62 |
| ATOM | 1547 | O | TYR | A | 728 | 12.588 | −14.401 | 23.028 | 1.00 | 14.23 |
| ATOM | 1548 | N | ILE | A | 729 | 14.636 | −13.522 | 22.723 | 1.00 | 12.98 |
| ATOM | 1549 | CA | ILE | A | 729 | 15.232 | −14.788 | 22.327 | 1.00 | 13.19 |
| ATOM | 1550 | CB | ILE | A | 729 | 16.723 | −14.595 | 21.983 | 1.00 | 14.56 |
| ATOM | 1551 | CG2 | ILE | A | 729 | 17.395 | −15.942 | 21.797 | 1.00 | 19.39 |
| ATOM | 1552 | CG1 | ILE | A | 729 | 16.849 | −13.727 | 20.727 | 1.00 | 18.02 |
| ATOM | 1553 | CD1 | ILE | A | 729 | 18.268 | −13.309 | 20.406 | 1.00 | 26.38 |
| ATOM | 1554 | C | ILE | A | 729 | 15.088 | −15.826 | 23.439 | 1.00 | 15.70 |
| ATOM | 1555 | O | ILE | A | 729 | 14.830 | −17.001 | 23.178 | 1.00 | 14.36 |
| ATOM | 1556 | N | LYS | A | 730 | 15.227 | −15.375 | 24.681 | 1.00 | 15.21 |
| ATOM | 1557 | CA | LYS | A | 730 | 15.127 | −16.257 | 25.837 | 1.00 | 16.44 |
| ATOM | 1558 | CB | LYS | A | 730 | 15.657 | −15.528 | 27.075 | 1.00 | 21.43 |
| ATOM | 1559 | CG | LYS | A | 730 | 15.494 | −16.291 | 28.376 | 1.00 | 27.38 |
| ATOM | 1560 | CD | LYS | A | 730 | 16.133 | −15.528 | 29.523 | 1.00 | 31.22 |
| ATOM | 1561 | CE | LYS | A | 730 | 15.975 | −16.267 | 30.839 | 1.00 | 36.43 |
| ATOM | 1562 | NZ | LYS | A | 730 | 16.672 | −15.556 | 31.947 | 1.00 | 41.27 |
| ATOM | 1563 | C | LYS | A | 730 | 13.727 | −16.802 | 26.133 | 1.00 | 16.48 |
| ATOM | 1564 | O | LYS | A | 730 | 13.573 | −17.972 | 26.498 | 1.00 | 17.06 |
| ATOM | 1565 | N | ARG | A | 731 | 12.707 | −15.969 | 25.968 | 1.00 | 14.55 |
| ATOM | 1566 | CA | ARG | A | 731 | 11.347 | −16.395 | 26.283 | 1.00 | 15.57 |
| ATOM | 1567 | CB | ARG | A | 731 | 10.622 | −15.291 | 27.060 | 1.00 | 19.59 |
| ATOM | 1568 | CG | ARG | A | 731 | 11.309 | −14.842 | 28.335 | 1.00 | 18.25 |
| ATOM | 1569 | CD | ARG | A | 731 | 10.437 | −13.846 | 29.086 | 1.00 | 33.93 |
| ATOM | 1570 | NE | ARG | A | 731 | 9.209 | −14.462 | 29.588 | 1.00 | 32.78 |
| ATOM | 1571 | CZ | ARG | A | 731 | 8.168 | −13.781 | 30.058 | 1.00 | 29.98 |
| ATOM | 1572 | NH1 | ARG | A | 731 | 8.197 | −12.456 | 30.091 | 1.00 | 30.21 |
| ATOM | 1573 | NH2 | ARG | A | 731 | 7.097 | −14.426 | 30.501 | 1.00 | 36.09 |
| ATOM | 1574 | C | ARG | A | 731 | 10.461 | −16.807 | 25.118 | 1.00 | 12.75 |
| ATOM | 1575 | O | ARG | A | 731 | 9.436 | −17.454 | 25.325 | 1.00 | 15.30 |
| ATOM | 1576 | N | ARG | A | 732 | 10.841 | −16.445 | 23.897 | 1.00 | 13.21 |
| ATOM | 1577 | CA | ARG | A | 732 | 10.002 | −16.759 | 22.751 | 1.00 | 11.06 |
| ATOM | 1578 | CB | ARG | A | 732 | 10.589 | −16.162 | 21.474 | 1.00 | 10.97 |
| ATOM | 1579 | CG | ARG | A | 732 | 11.872 | −16.812 | 21.021 | 1.00 | 12.09 |
| ATOM | 1580 | CD | ARG | A | 732 | 12.325 | −16.192 | 19.724 | 1.00 | 12.79 |
| ATOM | 1581 | NE | ARG | A | 732 | 13.525 | −16.822 | 19.193 | 1.00 | 13.80 |
| ATOM | 1582 | CZ | ARG | A | 732 | 13.919 | −16.704 | 17.930 | 1.00 | 20.20 |
| ATOM | 1583 | NH1 | ARG | A | 732 | 13.201 | −15.980 | 17.077 | 1.00 | 23.11 |
| ATOM | 1584 | NH2 | ARG | A | 732 | 15.025 | −17.307 | 17.518 | 1.00 | 22.17 |
| ATOM | 1585 | C | ARG | A | 732 | 9.751 | −18.245 | 22.549 | 1.00 | 11.37 |
| ATOM | 1586 | O | ARG | A | 732 | 8.704 | −18.625 | 22.031 | 1.00 | 11.95 |
| ATOM | 1587 | N | GLY | A | 733 | 10.703 | −19.078 | 22.962 | 1.00 | 13.25 |
| ATOM | 1588 | CA | GLY | A | 733 | 10.543 | −20.512 | 22.804 | 1.00 | 13.40 |
| ATOM | 1589 | C | GLY | A | 733 | 9.298 | −21.009 | 23.508 | 1.00 | 14.10 |
| ATOM | 1590 | O | GLY | A | 733 | 8.543 | −21.823 | 22.970 | 1.00 | 15.18 |
| ATOM | 1591 | N | GLU | A | 734 | 9.080 | −20.512 | 24.719 | 1.00 | 11.89 |
| ATOM | 1592 | CA | GLU | A | 734 | 7.919 | −20.900 | 25.505 | 1.00 | 14.43 |
| ATOM | 1593 | CB | GLU | A | 734 | 7.988 | −20.264 | 26.895 | 1.00 | 14.05 |
| ATOM | 1594 | CG | GLU | A | 734 | 6.675 | −20.312 | 27.661 | 1.00 | 18.08 |
| ATOM | 1595 | CD | GLU | A | 734 | 6.810 | −19.828 | 29.091 | 1.00 | 21.97 |
| ATOM | 1596 | OE1 | GLU | A | 734 | 7.742 | −19.048 | 29.372 | 1.00 | 19.31 |
| ATOM | 1597 | OE2 | GLU | A | 734 | 5.977 | −20.221 | 29.932 | 1.00 | 32.96 |
| ATOM | 1598 | C | GLU | A | 734 | 6.632 | −20.478 | 24.806 | 1.00 | 14.65 |
| ATOM | 1599 | O | GLU | A | 734 | 5.663 | −21.235 | 24.760 | 1.00 | 14.67 |
| ATOM | 1600 | N | PHE | A | 735 | 6.631 | −19.264 | 24.267 | 1.00 | 15.01 |
| ATOM | 1601 | CA | PHE | A | 735 | 5.470 | −18.729 | 23.566 | 1.00 | 11.90 |
| ATOM | 1602 | CB | PHE | A | 735 | 5.743 | −17.278 | 23.157 | 1.00 | 11.17 |
| ATOM | 1603 | CG | PHE | A | 735 | 4.632 | −16.635 | 22.366 | 1.00 | 16.06 |
| ATOM | 1604 | CD1 | PHE | A | 735 | 3.354 | −16.515 | 22.892 | 1.00 | 16.32 |
| ATOM | 1605 | CD2 | PHE | A | 735 | 4.885 | −16.102 | 21.111 | 1.00 | 13.68 |
| ATOM | 1606 | CE1 | PHE | A | 735 | 2.355 | −15.869 | 22.178 | 1.00 | 13.80 |
| ATOM | 1607 | CE2 | PHE | A | 735 | 3.897 | −15.458 | 20.400 | 1.00 | 12.16 |
| ATOM | 1608 | CZ | PHE | A | 735 | 2.631 | −15.340 | 20.931 | 1.00 | 14.03 |
| ATOM | 1609 | C | PHE | A | 735 | 5.142 | −19.566 | 22.329 | 1.00 | 13.78 |
| ATOM | 1610 | O | PHE | A | 735 | 3.989 | −19.946 | 22.117 | 1.00 | 13.01 |
| ATOM | 1611 | N | PHE | A | 736 | 6.156 | −19.853 | 21.517 | 1.00 | 12.56 |
| ATOM | 1612 | CA | PHE | A | 736 | 5.952 | −20.632 | 20.299 | 1.00 | 14.09 |
| ATOM | 1613 | CB | PHE | A | 736 | 7.242 | −20.691 | 19.472 | 1.00 | 15.66 |
| ATOM | 1614 | CG | PHE | A | 736 | 7.752 | −19.342 | 19.031 | 1.00 | 17.77 |
| ATOM | 1615 | CD1 | PHE | A | 736 | 6.888 | −18.268 | 18.894 | 1.00 | 16.30 |
| ATOM | 1616 | CD2 | PHE | A | 736 | 9.092 | −19.166 | 18.701 | 1.00 | 14.18 |
| ATOM | 1617 | CE1 | PHE | A | 736 | 7.345 | −17.040 | 18.435 | 1.00 | 13.31 |
| ATOM | 1618 | CE2 | PHE | A | 736 | 9.558 | −17.939 | 18.242 | 1.00 | 13.12 |
| ATOM | 1619 | CZ | PHE | A | 736 | 8.686 | −16.877 | 18.106 | 1.00 | 17.60 |
| ATOM | 1620 | C | PHE | A | 736 | 5.483 | −22.054 | 20.598 | 1.00 | 14.02 |
| ATOM | 1621 | O | PHE | A | 736 | 4.653 | −22.608 | 19.874 | 1.00 | 15.27 |
| ATOM | 1622 | N | GLU | A | 737 | 6.022 | −22.640 | 21.661 | 1.00 | 15.53 |
| ATOM | 1623 | CA | GLU | A | 737 | 5.667 | −24.000 | 22.047 | 1.00 | 16.49 |
| ATOM | 1624 | CB | GLU | A | 737 | 6.598 | −24.493 | 23.155 | 1.00 | 18.16 |
| ATOM | 1625 | CG | GLU | A | 737 | 6.296 | −25.908 | 23.623 | 1.00 | 32.20 |
| ATOM | 1626 | CD | GLU | A | 737 | 7.266 | −26.397 | 24.685 | 1.00 | 44.41 |
| ATOM | 1627 | OE1 | GLU | A | 737 | 7.368 | −25.748 | 25.748 | 1.00 | 43.95 |
| ATOM | 1628 | OE2 | GLU | A | 737 | 7.926 | −27.433 | 24.457 | 1.00 | 45.00 |
| ATOM | 1629 | C | GLU | A | 737 | 4.219 | −24.105 | 22.505 | 1.00 | 16.86 |
| ATOM | 1630 | O | GLU | A | 737 | 3.517 | −25.050 | 22.137 | 1.00 | 18.64 |
| ATOM | 1631 | N | LEU | A | 738 | 3.774 | −23.144 | 23.308 | 1.00 | 15.93 |
| ATOM | 1632 | CA | LEU | A | 738 | 2.396 | −23.140 | 23.791 | 1.00 | 14.96 |
| ATOM | 1633 | CB | LEU | A | 738 | 2.137 | −21.910 | 24.665 | 1.00 | 13.51 |
| ATOM | 1634 | CG | LEU | A | 738 | 2.817 | −21.890 | 26.037 | 1.00 | 13.44 |
| ATOM | 1635 | CD1 | LEU | A | 738 | 2.813 | −20.474 | 26.589 | 1.00 | 14.08 |
| ATOM | 1636 | CD2 | LEU | A | 738 | 2.103 | −22.844 | 26.987 | 1.00 | 21.77 |
| ATOM | 1637 | C | LEU | A | 738 | 1.435 | −23.137 | 22.610 | 1.00 | 15.65 |
| ATOM | 1638 | O | LEU | A | 738 | 0.431 | −23.854 | 22.610 | 1.00 | 17.64 |
| ATOM | 1639 | N | ILE | A | 739 | 1.743 | −22.323 | 21.604 | 1.00 | 13.75 |
| ATOM | 1640 | CA | ILE | A | 739 | 0.904 | −22.236 | 20.415 | 1.00 | 14.28 |
| ATOM | 1641 | CB | ILE | A | 739 | 1.388 | −21.112 | 19.464 | 1.00 | 12.79 |
| ATOM | 1642 | CG2 | ILE | A | 739 | 0.686 | −21.224 | 18.118 | 1.00 | 23.84 |
| ATOM | 1643 | CG1 | ILE | A | 739 | 1.122 | −19.744 | 20.096 | 1.00 | 16.58 |
| ATOM | 1644 | CD1 | ILE | A | 739 | 1.656 | −18.576 | 19.279 | 1.00 | 13.81 |
| ATOM | 1645 | C | ILE | A | 739 | 0.925 | −23.557 | 19.654 | 1.00 | 16.17 |
| ATOM | 1646 | O | ILE | A | 739 | −0.126 | −24.107 | 19.309 | 1.00 | 18.24 |
| ATOM | 1647 | N | ARG | A | 740 | 2.124 | −24.075 | 19.410 | 1.00 | 15.35 |
| ATOM | 1648 | CA | ARG | A | 740 | 2.274 | −25.322 | 18.671 | 1.00 | 18.48 |
| ATOM | 1649 | CB | ARG | A | 740 | 3.758 | −25.648 | 18.474 | 1.00 | 23.15 |
| ATOM | 1650 | CG | ARG | A | 740 | 3.996 | −26.804 | 17.508 | 1.00 | 35.46 |
| ATOM | 1651 | CD | ARG | A | 740 | 5.475 | −27.059 | 17.224 | 1.00 | 39.66 |
| ATOM | 1652 | NE | ARG | A | 740 | 6.174 | −27.694 | 18.340 | 1.00 | 40.53 |
| ATOM | 1653 | CZ | ARG | A | 740 | 6.703 | −27.043 | 19.371 | 1.00 | 42.20 |
| ATOM | 1654 | NH1 | ARG | A | 740 | 6.623 | −25.721 | 19.441 | 1.00 | 52.88 |
| ATOM | 1655 | NH2 | ARG | A | 740 | 7.317 | −27.718 | 20.335 | 1.00 | 44.19 |
| ATOM | 1656 | C | ARG | A | 740 | 1.582 | −26.495 | 19.360 | 1.00 | 19.41 |
| ATOM | 1657 | O | ARG | A | 740 | 1.093 | −27.410 | 18.698 | 1.00 | 18.06 |
| ATOM | 1658 | N | LYS | A | 741 | 1.539 | −26.470 | 20.686 | 1.00 | 18.03 |
| ATOM | 1659 | CA | LYS | A | 741 | 0.906 | −27.553 | 21.430 | 1.00 | 18.11 |
| ATOM | 1660 | CB | LYS | A | 741 | 1.696 | −27.853 | 22.699 | 1.00 | 14.50 |
| ATOM | 1661 | CG | LYS | A | 741 | 3.074 | −28.426 | 22.451 | 1.00 | 21.67 |
| ATOM | 1662 | CD | LYS | A | 741 | 3.717 | −28.812 | 23.763 | 1.00 | 23.36 |
| ATOM | 1663 | CE | LYS | A | 741 | 5.087 | −29.430 | 23.558 | 1.00 | 27.38 |
| ATOM | 1664 | NZ | LYS | A | 741 | 5.715 | −29.747 | 24.871 | 1.00 | 23.91 |
| ATOM | 1665 | C | LYS | A | 741 | −0.540 | −27.248 | 21.798 | 1.00 | 17.80 |
| ATOM | 1666 | O | LYS | A | 741 | −1.134 | −27.940 | 22.623 | 1.00 | 18.63 |
| ATOM | 1667 | N | ASN | A | 742 | −1.102 | −26.214 | 21.183 | 1.00 | 17.40 |
| ATOM | 1668 | CA | ASN | A | 742 | −2.478 | −25.822 | 21.456 | 1.00 | 17.84 |
| ATOM | 1669 | CB | ASN | A | 742 | −3.443 | −26.841 | 20.849 | 1.00 | 16.88 |
| ATOM | 1670 | CG | ASN | A | 742 | −3.442 | −26.801 | 19.335 | 1.00 | 15.02 |
| ATOM | 1671 | OD1 | ASN | A | 742 | −3.741 | −25.772 | 18.733 | 1.00 | 28.97 |
| ATOM | 1672 | ND2 | ASN | A | 742 | −3.100 | −27.923 | 18.709 | 1.00 | 20.44 |
| ATOM | 1673 | C | ASN | A | 742 | −2.734 | −25.671 | 22.949 | 1.00 | 17.96 |
| ATOM | 1674 | O | ASN | A | 742 | −3.727 | −26.174 | 23.486 | 1.00 | 17.06 |
| ATOM | 1675 | N | GLN | A | 743 | −1.827 | −24.964 | 23.615 | 1.00 | 16.68 |
| ATOM | 1676 | CA | GLN | A | 743 | −1.937 | −24.727 | 25.045 | 1.00 | 17.92 |
| ATOM | 1677 | CB | GLN | A | 743 | −0.738 | −25.338 | 25.777 | 1.00 | 20.38 |
| ATOM | 1678 | CG | GLN | A | 743 | −0.461 | −26.787 | 25.408 | 1.00 | 28.60 |
| ATOM | 1679 | CD | GLN | A | 743 | 0.746 | −27.350 | 26.131 | 1.00 | 30.94 |
| ATOM | 1680 | OE1 | GLN | A | 743 | 1.775 | −26.682 | 26.255 | 1.00 | 32.52 |
| ATOM | 1681 | NE2 | GLN | A | 743 | 0.634 | −28.588 | 26.602 | 1.00 | 20.21 |
| ATOM | 1682 | C | GLN | A | 743 | −2.002 | −23.233 | 25.341 | 1.00 | 16.35 |
| ATOM | 1683 | O | GLN | A | 743 | −2.175 | −22.836 | 26.490 | 1.00 | 18.44 |
| ATOM | 1684 | N | PHE | A | 744 | −1.877 | −22.407 | 24.307 | 1.00 | 16.26 |
| ATOM | 1685 | CA | PHE | A | 744 | −1.916 | −20.959 | 24.501 | 1.00 | 19.79 |
| ATOM | 1686 | CB | PHE | A | 744 | −1.627 | −20.229 | 23.184 | 1.00 | 21.58 |
| ATOM | 1687 | CG | PHE | A | 744 | −1.465 | −18.740 | 23.340 | 1.00 | 17.97 |
| ATOM | 1688 | CD1 | PHE | A | 744 | −0.441 | −18.216 | 24.117 | 1.00 | 20.04 |
| ATOM | 1689 | CD2 | PHE | A | 744 | −2.345 | −17.865 | 22.720 | 1.00 | 24.29 |
| ATOM | 1690 | CE1 | PHE | A | 744 | −0.298 | −16.847 | 24.271 | 1.00 | 24.80 |
| ATOM | 1691 | CE2 | PHE | A | 744 | −2.208 | −16.499 | 22.869 | 1.00 | 20.38 |
| ATOM | 1692 | CZ | PHE | A | 744 | −1.184 | −15.988 | 23.647 | 1.00 | 26.24 |
| ATOM | 1693 | C | PHE | A | 744 | −3.265 | −20.515 | 25.060 | 1.00 | 19.74 |
| ATOM | 1694 | O | PHE | A | 744 | −4.316 | −20.860 | 24.526 | 1.00 | 21.03 |
| ATOM | 1695 | N | ASN | A | 745 | −3.221 | −19.746 | 26.142 | 1.00 | 19.11 |
| ATOM | 1696 | CA | ASN | A | 745 | −4.427 | −19.260 | 26.791 | 1.00 | 18.83 |
| ATOM | 1697 | CB | ASN | A | 745 | −4.893 | −20.270 | 27.839 | 1.00 | 14.11 |
| ATOM | 1698 | CG | ASN | A | 745 | −6.067 | −19.764 | 28.653 | 1.00 | 17.45 |
| ATOM | 1699 | OD1 | ASN | A | 745 | −6.924 | −19.042 | 28.143 | 1.00 | 22.19 |
| ATOM | 1700 | ND2 | ASN | A | 745 | −6.120 | −20.152 | 29.921 | 1.00 | 30.84 |
| ATOM | 1701 | C | ASN | A | 745 | −4.183 | −17.906 | 27.446 | 1.00 | 17.46 |
| ATOM | 1702 | O | ASN | A | 745 | −3.518 | −17.818 | 28.481 | 1.00 | 17.84 |
| ATOM | 1703 | N | LEU | A | 746 | −4.720 | −16.857 | 26.826 | 1.00 | 16.56 |
| ATOM | 1704 | CA | LEU | A | 746 | −4.573 | −15.497 | 27.323 | 1.00 | 18.48 |
| ATOM | 1705 | CB | LEU | A | 746 | −5.163 | −14.495 | 26.323 | 1.00 | 15.11 |
| ATOM | 1706 | CG | LEU | A | 746 | −4.319 | −14.192 | 25.082 | 1.00 | 17.98 |
| ATOM | 1707 | CD1 | LEU | A | 746 | −5.031 | −13.165 | 24.217 | 1.00 | 16.50 |
| ATOM | 1708 | CD2 | LEU | A | 746 | −2.960 | −13.664 | 25.507 | 1.00 | 17.71 |
| ATOM | 1709 | C | LEU | A | 746 | −5.212 | −15.284 | 28.688 | 1.00 | 21.08 |
| ATOM | 1710 | O | LEU | A | 746 | −4.911 | −14.304 | 29.366 | 1.00 | 20.60 |
| ATOM | 1711 | N | GLU | A | 747 | −6.098 | −16.192 | 29.089 | 1.00 | 21.14 |
| ATOM | 1712 | CA | GLU | A | 747 | −6.748 | −16.075 | 30.389 | 1.00 | 21.90 |
| ATOM | 1713 | CB | GLU | A | 747 | −7.916 | −17.058 | 30.504 | 1.00 | 20.33 |
| ATOM | 1714 | CG | GLU | A | 747 | −9.179 | −16.586 | 29.813 | 1.00 | 31.82 |
| ATOM | 1715 | CD | GLU | A | 747 | −10.324 | −17.569 | 29.959 | 1.00 | 40.12 |
| ATOM | 1716 | OE1 | GLU | A | 747 | −10.550 | −18.057 | 31.086 | 1.00 | 43.30 |
| ATOM | 1717 | OE2 | GLU | A | 747 | −11.003 | −17.846 | 28.949 | 1.00 | 40.94 |
| ATOM | 1718 | C | GLU | A | 747 | −5.740 | −16.331 | 31.498 | 1.00 | 22.98 |
| ATOM | 1719 | O | GLU | A | 747 | −5.885 | −15.826 | 32.612 | 1.00 | 23.46 |
| ATOM | 1720 | N | ASP | A | 748 | −4.719 | −17.124 | 31.188 | 1.00 | 21.44 |
| ATOM | 1721 | CA | ASP | A | 748 | −3.670 | −17.424 | 32.151 | 1.00 | 21.15 |
| ATOM | 1722 | CB | ASP | A | 748 | −2.800 | −18.579 | 31.651 | 1.00 | 19.25 |
| ATOM | 1723 | CG | ASP | A | 748 | −1.656 | −18.899 | 32.595 | 1.00 | 19.90 |
| ATOM | 1724 | OD1 | ASP | A | 748 | −0.710 | −18.088 | 32.692 | 1.00 | 26.86 |
| ATOM | 1725 | OD2 | ASP | A | 748 | −1.704 | −19.962 | 33.246 | 1.00 | 42.46 |
| ATOM | 1726 | C | ASP | A | 748 | −2.819 | −16.171 | 32.314 | 1.00 | 20.94 |
| ATOM | 1727 | O | ASP | A | 748 | −2.240 | −15.677 | 31.351 | 1.00 | 19.01 |
| ATOM | 1728 | N | PRO | A | 749 | −2.742 | −15.637 | 33.541 | 1.00 | 18.53 |
| ATOM | 1729 | CD | PRO | A | 749 | −3.443 | −16.108 | 34.750 | 1.00 | 26.08 |
| ATOM | 1730 | CA | PRO | A | 749 | −1.958 | −14.432 | 33.825 | 1.00 | 19.83 |
| ATOM | 1731 | CB | PRO | A | 749 | −2.009 | −14.346 | 35.347 | 1.00 | 25.90 |
| ATOM | 1732 | CG | PRO | A | 749 | −3.377 | −14.896 | 35.654 | 1.00 | 25.60 |
| ATOM | 1733 | C | PRO | A | 749 | −0.528 | −14.476 | 33.286 | 1.00 | 17.96 |
| ATOM | 1734 | O | PRO | A | 749 | −0.047 | −13.506 | 32.708 | 1.00 | 18.09 |
| ATOM | 1735 | N | HIS | A | 750 | 0.149 | −15.603 | 33.475 | 1.00 | 17.27 |
| ATOM | 1736 | CA | HIS | A | 750 | 1.520 | −15.737 | 32.998 | 1.00 | 16.70 |
| ATOM | 1737 | CB | HIS | A | 750 | 2.138 | −17.039 | 33.509 | 1.00 | 14.57 |
| ATOM | 1738 | CG | HIS | A | 750 | 3.506 | −17.301 | 32.975 | 1.00 | 13.17 |
| ATOM | 1739 | CD2 | HIS | A | 750 | 4.710 | −16.780 | 33.299 | 1.00 | 15.22 |
| ATOM | 1740 | ND1 | HIS | A | 750 | 3.748 | −18.181 | 31.933 | 1.00 | 20.55 |
| ATOM | 1741 | CE1 | HIS | A | 750 | 5.030 | −18.183 | 31.649 | 1.00 | 21.44 |
| ATOM | 1742 | NE2 | HIS | A | 750 | 5.644 | −17.338 | 32.465 | 1.00 | 27.33 |
| ATOM | 1743 | C | HIS | A | 750 | 1.615 | −15.691 | 31.475 | 1.00 | 16.04 |
| ATOM | 1744 | O | HIS | A | 750 | 2.521 | −15.069 | 30.925 | 1.00 | 16.50 |
| ATOM | 1745 | N | GLU | A | 751 | 0.680 | −16.346 | 30.794 | 1.00 | 14.76 |
| ATOM | 1746 | CA | GLU | A | 751 | 0.689 | −16.359 | 29.333 | 1.00 | 14.97 |
| ATOM | 1747 | CB | GLU | A | 751 | −0.269 | −17.427 | 28.798 | 1.00 | 11.89 |
| ATOM | 1748 | CG | GLU | A | 751 | 0.173 | −18.848 | 29.123 | 1.00 | 16.94 |
| ATOM | 1749 | CD | GLU | A | 751 | −0.601 | −19.892 | 28.354 | 1.00 | 17.06 |
| ATOM | 1750 | OE1 | GLU | A | 751 | −0.804 | −19.697 | 27.140 | 1.00 | 17.15 |
| ATOM | 1751 | OE2 | GLU | A | 751 | −0.993 | −20.912 | 28.959 | 1.00 | 27.83 |
| ATOM | 1752 | C | GLU | A | 751 | 0.325 | −14.988 | 28.764 | 1.00 | 14.11 |
| ATOM | 1753 | O | GLU | A | 751 | 0.782 | −14.616 | 27.685 | 1.00 | 14.18 |
| ATOM | 1754 | N | LYS | A | 752 | −0.501 | −14.248 | 29.498 | 1.00 | 13.06 |
| ATOM | 1755 | CA | LYS | A | 752 | −0.900 | −12.910 | 29.092 | 1.00 | 11.63 |
| ATOM | 1756 | CB | LYS | A | 752 | −1.984 | −12.370 | 30.041 | 1.00 | 11.74 |
| ATOM | 1757 | CG | LYS | A | 752 | −2.200 | −10.865 | 29.981 | 1.00 | 11.33 |
| ATOM | 1758 | CD | LYS | A | 752 | −2.667 | −10.402 | 28.608 | 1.00 | 18.51 |
| ATOM | 1759 | CE | LYS | A | 752 | −3.921 | −11.138 | 28.184 | 1.00 | 31.49 |
| ATOM | 1760 | NZ | LYS | A | 752 | −4.536 | −10.523 | 26.978 | 1.00 | 47.29 |
| ATOM | 1761 | C | LYS | A | 752 | 0.362 | −12.047 | 29.176 | 1.00 | 14.58 |
| ATOM | 1762 | O | LYS | A | 752 | 0.665 | −11.293 | 28.249 | 1.00 | 11.11 |
| ATOM | 1763 | N | GLU | A | 753 | 1.093 | −12.196 | 30.270 | 1.00 | 12.46 |
| ATOM | 1764 | CA | GLU | A | 753 | 2.315 | −11.409 | 30.445 | 1.00 | 13.89 |
| ATOM | 1765 | CB | GLU | A | 753 | 2.939 | −11.745 | 31.801 | 1.00 | 15.41 |
| ATOM | 1766 | CG | GLU | A | 753 | 4.187 | −10.950 | 32.098 | 1.00 | 17.51 |
| ATOM | 1767 | CD | GLU | A | 753 | 4.946 | −11.478 | 33.300 | 1.00 | 36.21 |
| ATOM | 1768 | OE1 | GLU | A | 753 | 4.335 | −11.579 | 34.384 | 1.00 | 28.23 |
| ATOM | 1769 | OE2 | GLU | A | 753 | 6.150 | −11.782 | 33.162 | 1.00 | 36.81 |
| ATOM | 1770 | C | GLU | A | 753 | 3.267 | −11.729 | 29.313 | 1.00 | 14.45 |
| ATOM | 1771 | O | GLU | A | 753 | 3.852 | −10.839 | 28.671 | 1.00 | 11.34 |
| ATOM | 1772 | N | LEU | A | 754 | 3.441 | −13.018 | 29.035 | 1.00 | 12.90 |
| ATOM | 1773 | CA | LEU | A | 754 | 4.329 | −13.471 | 27.974 | 1.00 | 13.51 |
| ATOM | 1774 | CB | LEU | A | 754 | 4.377 | −15.011 | 27.908 | 1.00 | 13.76 |
| ATOM | 1775 | CG | LEU | A | 754 | 5.196 | −15.682 | 26.793 | 1.00 | 15.32 |
| ATOM | 1776 | CD1 | LEU | A | 754 | 6.663 | −15.286 | 26.877 | 1.00 | 15.56 |
| ATOM | 1777 | CD2 | LEU | A | 754 | 5.044 | −17.193 | 26.930 | 1.00 | 14.90 |
| ATOM | 1778 | C | LEU | A | 754 | 3.915 | −12.900 | 26.616 | 1.00 | 12.21 |
| ATOM | 1779 | O | LEU | A | 754 | 4.765 | −12.478 | 25.821 | 1.00 | 11.37 |
| ATOM | 1780 | N | PHE | A | 755 | 2.613 | −12.867 | 26.351 | 1.00 | 11.00 |
| ATOM | 1781 | CA | PHE | A | 755 | 2.119 | −12.336 | 25.088 | 1.00 | 9.92 |
| ATOM | 1782 | CB | PHE | A | 755 | 0.609 | −12.551 | 24.966 | 1.00 | 12.44 |
| ATOM | 1783 | CG | PHE | A | 755 | 0.002 | −11.867 | 23.780 | 1.00 | 11.67 |
| ATOM | 1784 | CD1 | PHE | A | 755 | 0.338 | −12.259 | 22.492 | 1.00 | 13.36 |
| ATOM | 1785 | CD2 | PHE | A | 755 | −0.858 | −10.792 | 23.948 | 1.00 | 9.93 |
| ATOM | 1786 | CE1 | PHE | A | 755 | −0.170 | −11.590 | 21.393 | 1.00 | 14.56 |
| ATOM | 1787 | CE2 | PHE | A | 755 | −1.369 | −10.118 | 22.854 | 1.00 | 13.93 |
| ATOM | 1788 | CZ | PHE | A | 755 | −1.021 | −10.517 | 21.574 | 1.00 | 18.65 |
| ATOM | 1789 | C | PHE | A | 755 | 2.431 | −10.840 | 24.972 | 1.00 | 9.95 |
| ATOM | 1790 | O | PHE | A | 755 | 2.856 | −10.367 | 23.916 | 1.00 | 10.28 |
| ATOM | 1791 | N | LEU | A | 756 | 2.225 | −10.099 | 26.057 | 1.00 | 9.98 |
| ATOM | 1792 | CA | LEU | A | 756 | 2.489 | −8.664 | 26.034 | 1.00 | 9.03 |
| ATOM | 1793 | CB | LEU | A | 756 | 2.102 | −8.017 | 27.370 | 1.00 | 9.14 |
| ATOM | 1794 | CG | LEU | A | 756 | 0.610 | −8.119 | 27.706 | 1.00 | 13.27 |
| ATOM | 1795 | CD1 | LEU | A | 756 | 0.331 | −7.487 | 29.065 | 1.00 | 21.21 |
| ATOM | 1796 | CD2 | LEU | A | 756 | −0.198 | −7.432 | 26.613 | 1.00 | 18.57 |
| ATOM | 1797 | C | LEU | A | 756 | 3.959 | −8.410 | 25.721 | 1.00 | 9.36 |
| ATOM | 1798 | O | LEU | A | 756 | 4.291 | −7.437 | 25.045 | 1.00 | 8.42 |
| ATOM | 1799 | N | ALA | A | 757 | 4.844 | −9.285 | 26.197 | 1.00 | 7.41 |
| ATOM | 1800 | CA | ALA | A | 757 | 6.267 | −9.118 | 25.915 | 1.00 | 10.17 |
| ATOM | 1801 | CB | ALA | A | 757 | 7.096 | −10.112 | 26.720 | 1.00 | 10.57 |
| ATOM | 1802 | C | ALA | A | 757 | 6.514 | −9.315 | 24.421 | 1.00 | 9.99 |
| ATOM | 1803 | O | ALA | A | 757 | 7.254 | −8.548 | 23.798 | 1.00 | 10.22 |
| ATOM | 1804 | N | MET | A | 758 | 5.890 | −10.341 | 23.845 | 1.00 | 8.41 |
| ATOM | 1805 | CA | MET | A | 758 | 6.046 | −10.627 | 22.422 | 1.00 | 8.55 |
| ATOM | 1806 | CB | MET | A | 758 | 5.348 | −11.946 | 22.061 | 1.00 | 8.57 |
| ATOM | 1807 | CG | MET | A | 758 | 5.898 | −13.175 | 22.783 | 1.00 | 9.97 |
| ATOM | 1808 | SD | MET | A | 758 | 7.613 | −13.597 | 22.367 | 1.00 | 15.99 |
| ATOM | 1809 | CE | MET | A | 758 | 7.436 | −14.226 | 20.702 | 1.00 | 36.66 |
| ATOM | 1810 | C | MET | A | 758 | 5.469 | −9.499 | 21.572 | 1.00 | 9.31 |
| ATOM | 1811 | O | MET | A | 758 | 6.035 | −9.134 | 20.534 | 1.00 | 8.67 |
| ATOM | 1812 | N | LEU | A | 759 | 4.334 | −8.962 | 22.008 | 1.00 | 10.68 |
| ATOM | 1813 | CA | LEU | A | 759 | 3.682 | −7.877 | 21.290 | 1.00 | 7.26 |
| ATOM | 1814 | CB | LEU | A | 759 | 2.348 | −7.536 | 21.954 | 1.00 | 6.70 |
| ATOM | 1815 | CG | LEU | A | 759 | 1.521 | −6.419 | 21.316 | 1.00 | 9.88 |
| ATOM | 1816 | CD1 | LEU | A | 759 | 1.217 | −6.751 | 19.858 | 1.00 | 13.46 |
| ATOM | 1817 | CD2 | LEU | A | 759 | 0.232 | −6.241 | 22.103 | 1.00 | 8.91 |
| ATOM | 1818 | C | LEU | A | 759 | 4.589 | −6.650 | 21.269 | 1.00 | 10.65 |
| ATOM | 1819 | O | LEU | A | 759 | 4.677 | −5.945 | 20.259 | 1.00 | 8.66 |
| ATOM | 1820 | N | MET | A | 760 | 5.258 | −6.388 | 22.387 | 1.00 | 8.87 |
| ATOM | 1821 | CA | MET | A | 760 | 6.168 | −5.251 | 22.454 | 1.00 | 9.10 |
| ATOM | 1822 | CB | MET | A | 760. | 6.760 | −5.114 | 23.859 | 1.00 | 9.37 |
| ATOM | 1823 | CG | MET | A | 760 | 5.831 | −4.491 | 24.889 | 1.00 | 7.94 |
| ATOM | 1824 | SD | MET | A | 760 | 5.392 | −2.762 | 24.513 | 1.00 | 11.15 |
| ATOM | 1825 | CE | MET | A | 760 | 6.981 | −1.951 | 24.748 | 1.00 | 10.58 |
| ATOM | 1826 | C | MET | A | 760 | 7.292 | −5.445 | 21.444 | 1.00 | 9.36 |
| ATOM | 1827 | O | MET | A | 760 | 7.693 | −4.501 | 20.757 | 1.00 | 8.44 |
| ATOM | 1828 | N | THR | A | 761 | 7.812 | −6.667 | 21.359 | 1.00 | 8.62 |
| ATOM | 1829 | CA | THR | A | 761 | 8.890 | −6.948 | 20.416 | 1.00 | 9.46 |
| ATOM | 1830 | CB | THR | A | 761 | 9.442 | −8.379 | 20.597 | 1.00 | 12.42 |
| ATOM | 1831 | OG1 | THR | A | 761 | 9.916 | −8.540 | 21.940 | 1.00 | 11.86 |
| ATOM | 1832 | CG2 | THR | A | 761 | 10.598 | −8.636 | 19.620 | 1.00 | 9.90 |
| ATOM | 1833 | C | THR | A | 761 | 8.389 | −6.785 | 18.984 | 1.00 | 10.31 |
| ATOM | 1834 | O | THR | A | 761 | 9.088 | −6.238 | 18.128 | 1.00 | 10.55 |
| ATOM | 1835 | N | ALA | A | 762 | 7.172 | −7.254 | 18.722 | 1.00 | 10.42 |
| ATOM | 1836 | CA | ALA | A | 762 | 6.598 | −7.148 | 17.389 | 1.00 | 7.73 |
| ATOM | 1837 | CB | ALA | A | 762 | 5.202 | −7.777 | 17.362 | 1.00 | 10.79 |
| ATOM | 1838 | C | ALA | A | 762 | 6.524 | −5.689 | 16.948 | 1.00 | 9.57 |
| ATOM | 1839 | O | ALA | A | 762 | 6.794 | −5.370 | 15.787 | 1.00 | 9.59 |
| ATOM | 1840 | N | CYS | A | 763 | 6.161 | −4.801 | 17.872 | 1.00 | 7.53 |
| ATOM | 1841 | CA | CYS | A | 763 | 6.063 | −3.378 | 17.551 | 1.00 | 7.89 |
| ATOM | 1842 | CB | CYS | A | 763 | 5.237 | −2.641 | 18.611 | 1.00 | 11.70 |
| ATOM | 1843 | SG | CYS | A | 763 | 3.503 | −3.143 | 18.670 | 1.00 | 11.19 |
| ATOM | 1844 | C | CYS | A | 763 | 7.441 | −2.744 | 17.443 | 1.00 | 9.03 |
| ATOM | 1845 | O | CYS | A | 763 | 7.689 | −1.911 | 16.574 | 1.00 | 10.26 |
| ATOM | 1846 | N | ASP | A | 764 | 8.340 | −3.157 | 18.329 | 1.00 | 8.73 |
| ATOM | 1847 | CA | ASP | A | 764 | 9.703 | −2.641 | 18.352 | 1.00 | 10.03 |
| ATOM | 1848 | CB | ASP | A | 764 | 10.448 | −3.269 | 19.541 | 1.00 | 11.85 |
| ATOM | 1849 | CG | ASP | A | 764 | 11.766 | −2.585 | 19.840 | 1.00 | 12.03 |
| ATOM | 1850 | OD1 | ASP | A | 764 | 11.963 | −1.451 | 19.361 | 1.00 | 11.46 |
| ATOM | 1851 | OD2 | ASP | A | 764 | 12.592 | −3.176 | 20.571 | 1.00 | 15.03 |
| ATOM | 1852 | C | ASP | A | 764 | 10.447 | −2.924 | 17.036 | 1.00 | 10.20 |
| ATOM | 1853 | O | ASP | A | 764 | 11.225 | −2.097 | 16.562 | 1.00 | 11.71 |
| ATOM | 1854 | N | LEU | A | 765 | 10.194 | −4.086 | 16.442 | 1.00 | 11.54 |
| ATOM | 1855 | CA | LEU | A | 765 | 10.867 | −4.474 | 15.202 | 1.00 | 11.71 |
| ATOM | 1856 | CB | LEU | A | 765 | 11.208 | −5.965 | 15.252 | 1.00 | 13.42 |
| ATOM | 1857 | CG | LEU | A | 765 | 12.017 | −6.454 | 16.453 | 1.00 | 15.02 |
| ATOM | 1858 | CD1 | LEU | A | 765 | 12.233 | −7.957 | 16.348 | 1.00 | 13.91 |
| ATOM | 1859 | CD2 | LEU | A | 765 | 13.345 | −5.721 | 16.499 | 1.00 | 16.31 |
| ATOM | 1860 | C | LEU | A | 765 | 10.049 | −4.221 | 13.941 | 1.00 | 12.10 |
| ATOM | 1861 | O | LEU | A | 765 | 10.490 | −4.560 | 12.837 | 1.00 | 11.30 |
| ATOM | 1862 | N | SER | A | 766 | 8.881 | −3.606 | 14.101 | 1.00 | 12.76 |
| ATOM | 1863 | CA | SER | A | 766 | 7.965 | −3.383 | 12.983 | 1.00 | 10.86 |
| ATOM | 1864 | CB | SER | A | 766 | 6.672 | −2.733 | 13.488 | 1.00 | 8.49 |
| ATOM | 1865 | OG | SER | A | 766 | 6.902 | −1.438 | 13.991 | 1.00 | 14.83 |
| ATOM | 1866 | C | SER | A | 766 | 8.446 | −2.657 | 11.729 | 1.00 | 7.60 |
| ATOM | 1867 | O | SER | A | 766 | 7.782 | −2.735 | 10.700 | 1.00 | 9.56 |
| ATOM | 1868 | N | ALA | A | 767 | 9.575 | −1.956 | 11.787 | 1.00 | 8.02 |
| ATOM | 1869 | CA | ALA | A | 767 | 10.073 | −1.296 | 10.581 | 1.00 | 9.45 |
| ATOM | 1870 | CB | ALA | A | 767 | 11.364 | −0.522 | 10.881 | 1.00 | 11.41 |
| ATOM | 1871 | C | ALA | A | 767 | 10.340 | −2.377 | 9.521 | 1.00 | 10.04 |
| ATOM | 1872 | O | ALA | A | 767 | 10.253 | −2.124 | 8.319 | 1.00 | 10.04 |
| ATOM | 1873 | N | ILE | A | 768 | 10.655 | −3.586 | 9.976 | 1.00 | 9.49 |
| ATOM | 1874 | CA | ILE | A | 768 | 10.942 | −4.694 | 9.066 | 1.00 | 12.99 |
| ATOM | 1875 | CB | ILE | A | 768 | 11.555 | −5.894 | 9.840 | 1.00 | 9.64 |
| ATOM | 1876 | CG2 | ILE | A | 768 | 10.473 | −6.631 | 10.612 | 1.00 | 7.91 |
| ATOM | 1877 | CG1 | ILE | A | 768 | 12.270 | −6.836 | 8.868 | 1.00 | 12.83 |
| ATOM | 1878 | CD1 | ILE | A | 768 | 13.471 | −6.199 | 8.190 | 1.00 | 15.69 |
| ATOM | 1879 | C | ILE | A | 768 | 9.704 | −5.173 | 8.296 | 1.00 | 11.57 |
| ATOM | 1880 | O | ILE | A | 768 | 9.812 | −5.986 | 7.376 | 1.00 | 12.45 |
| ATOM | 1881 | N | THR | A | 769 | 8.530 | −4.665 | 8.662 | 1.00 | 12.70 |
| ATOM | 1882 | CA | THR | A | 769 | 7.286 | −5.062 | 7.999 | 1.00 | 10.88 |
| ATOM | 1883 | CB | THR | A | 769 | 6.167 | −5.326 | 9.025 | 1.00 | 13.25 |
| ATOM | 1884 | OG1 | THR | A | 769 | 5.760 | −4.083 | 9.617 | 1.00 | 12.15 |
| ATOM | 1885 | CG2 | THR | A | 769 | 6.654 | −6.271 | 10.124 | 1.00 | 16.86 |
| ATOM | 1886 | C | THR | A | 769 | 6.762 | −3.991 | 7.036 | 1.00 | 12.18 |
| ATOM | 1887 | O | THR | A | 769 | 5.795 | −4.223 | 6.308 | 1.00 | 13.18 |
| ATOM | 1888 | N | LYS | A | 770 | 7.402 | −2.824 | 7.043 | 1.00 | 12.64 |
| ATOM | 1889 | CA | LYS | A | 770 | 6.986 | −1.686 | 6.216 | 1.00 | 10.43 |
| ATOM | 1890 | CB | LYS | A | 770 | 7.684 | −0.413 | 6.714 | 1.00 | 12.22 |
| ATOM | 1891 | CG | LYS | A | 770 | 7.375 | −0.029 | 8.165 | 1.00 | 7.87 |
| ATOM | 1892 | CD | LYS | A | 770 | 5.948 | 0.457 | 8.330 | 1.00 | 13.33 |
| ATOM | 1893 | CE | LYS | A | 770 | 5.644 | 0.766 | 9.794 | 1.00 | 14.40 |
| ATOM | 1894 | NZ | LYS | A | 770 | 4.263 | 1.293 | 9.973 | 1.00 | 14.45 |
| ATOM | 1895 | C | LYS | A | 770 | 7.247 | −1.829 | 4.716 | 1.00 | 11.91 |
| ATOM | 1896 | O | LYS | A | 770 | 8.069 | −2.638 | 4.290 | 1.00 | 13.63 |
| ATOM | 1897 | N | PRO | A | 771 | 6.529 | −1.041 | 3.892 | 1.00 | 11.52 |
| ATOM | 1898 | CD | PRO | A | 771 | 5.408 | −0.150 | 4.240 | 1.00 | 14.60 |
| ATOM | 1899 | CA | PRO | A | 771 | 6.718 | −1.096 | 2.441 | 1.00 | 12.80 |
| ATOM | 1900 | CB | PRO | A | 771 | 5.914 | 0.099 | 1.950 | 1.00 | 15.88 |
| ATOM | 1901 | CG | PRO | A | 771 | 4.751 | 0.098 | 2.886 | 1.00 | 11.15 |
| ATOM | 1902 | C | PRO | A | 771 | 8.206 | −0.933 | 2.183 | 1.00 | 14.66 |
| ATOM | 1903 | O | PRO | A | 771 | 8.880 | −0.201 | 2.908 | 1.00 | 15.01 |
| ATOM | 1904 | N | TRP | A | 772 | 8.711 | −1.604 | 1.156 | 1.00 | 14.26 |
| ATOM | 1905 | CA | TRP | A | 772 | 10.133 | −1.555 | 0.827 | 1.00 | 15.34 |
| ATOM | 1906 | CB | TRP | A | 772 | 10.367 | −2.121 | −0.578 | 1.00 | 14.10 |
| ATOM | 1907 | CG | TRP | A | 772 | 11.809 | −2.101 | −1.009 | 1.00 | 17.47 |
| ATOM | 1908 | CD2 | TRP | A | 772 | 12.931 | −2.659 | −0.307 | 1.00 | 17.61 |
| ATOM | 1909 | CE2 | TRP | A | 772 | 14.079 | −2.404 | −1.086 | 1.00 | 19.63 |
| ATOM | 1910 | CE3 | TRP | A | 772 | 13.076 | −3.348 | 0.901 | 1.00 | 16.69 |
| ATOM | 1911 | CD1 | TRP | A | 772 | 12.313 | −1.547 | −2.152 | 1.00 | 16.69 |
| ATOM | 1912 | NE1 | TRP | A | 772 | 13.675 | −1.725 | −2.204 | 1.00 | 24.41 |
| ATOM | 1913 | CZ2 | TRP | A | 772 | 15.354 | −2.816 | −0.694 | 1.00 | 24.29 |
| ATOM | 1914 | CZ3 | TRP | A | 772 | 14.344 | −3.755 | 1.289 | 1.00 | 17.48 |
| ATOM | 1915 | CH2 | TRP | A | 772 | 15.466 | −3.488 | 0.493 | 1.00 | 21.25 |
| ATOM | 1916 | C | TRP | A | 772 | 10.820 | −0.190 | 0.952 | 1.00 | 17.78 |
| ATOM | 1917 | O | TRP | A | 772 | 11.834 | −0.070 | 1.639 | 1.00 | 15.81 |
| ATOM | 1918 | N | PRO | A | 773 | 10.286 | 0.853 | 0.291 | 1.00 | 19.35 |
| ATOM | 1919 | CD | PRO | A | 773 | 9.132 | 0.877 | −0.624 | 1.00 | 18.85 |
| ATOM | 1920 | CA | PRO | A | 773 | 10.909 | 2.180 | 0.377 | 1.00 | 18.24 |
| ATOM | 1921 | CB | PRO | A | 773 | 9.926 | 3.068 | −0.382 | 1.00 | 21.17 |
| ATOM | 1922 | CG | PRO | A | 773 | 9.386 | 2.138 | −1.422 | 1.00 | 23.60 |
| ATOM | 1923 | C | PRO | A | 773 | 11.129 | 2.654 | 1.814 | 1.00 | 16.33 |
| ATOM | 1924 | O | PRO | A | 773 | 12.167 | 3.235 | 2.140 | 1.00 | 16.01 |
| ATOM | 1925 | N | ILE | A | 774 | 10.143 | 2.406 | 2.668 | 1.00 | 16.23 |
| ATOM | 1926 | CA | ILE | A | 774 | 10.228 | 2.805 | 4.066 | 1.00 | 15.30 |
| ATOM | 1927 | CB | ILE | A | 774 | 8.851 | 2.670 | 4.751 | 1.00 | 15.99 |
| ATOM | 1928 | CG2 | ILE | A | 774 | 8.977 | 2.922 | 6.250 | 1.00 | 13.24 |
| ATOM | 1929 | CG1 | ILE | A | 774 | 7.867 | 3.658 | 4.110 | 1.00 | 20.68 |
| ATOM | 1930 | CD1 | ILE | A | 774 | 6.441 | 3.501 | 4.588 | 1.00 | 21.74 |
| ATOM | 1931 | C | ILE | A | 774 | 11.264 | 1.958 | 4.804 | 1.00 | 16.31 |
| ATOM | 1932 | O | ILE | A | 774 | 12.133 | 2.491 | 5.497 | 1.00 | 15.63 |
| ATOM | 1933 | N | GLN | A | 775 | 11.173 | 0.640 | 4.650 | 1.00 | 13.80 |
| ATOM | 1934 | CA | GLN | A | 775 | 12.120 | −0.274 | 5.279 | 1.00 | 12.85 |
| ATOM | 1935 | CB | GLN | A | 775 | 11.786 | −1.727 | 4.901 | 1.00 | 13.34 |
| ATOM | 1936 | CG | GLN | A | 775 | 12.987 | −2.688 | 4.921 | 1.00 | 14.20 |
| ATOM | 1937 | CD | GLN | A | 775 | 13.582 | −2.897 | 6.305 | 1.00 | 21.70 |
| ATOM | 1938 | OE1 | GLN | A | 775 | 14.663 | −3.472 | 6.441 | 1.00 | 13.81 |
| ATOM | 1939 | NE2 | GLN | A | 775 | 12.880 | −2.440 | 7.336 | 1.00 | 14.50 |
| ATOM | 1940 | C | GLN | A | 775 | 13.549 | 0.057 | 4.877 | 1.00 | 13.13 |
| ATOM | 1941 | O | GLN | A | 775 | 14.458 | 0.060 | 5.694 | 1.00 | 12.67 |
| ATOM | 1942 | N | GLN | A | 776 | 13.749 | 0.352 | 3.590 | 1.00 | 10.04 |
| ATOM | 1943 | CA | GLN | A | 776 | 15.068 | 0.674 | 3.090 | 1.00 | 12.84 |
| ATOM | 1944 | CB | GLN | A | 776 | 15.004 | 0.820 | 1.564 | 1.00 | 19.00 |
| ATOM | 1945 | CG | GLN | A | 776 | 16.326 | 0.943 | 0.890 | 1.00 | 25.33 |
| ATOM | 1946 | CD | GLN | A | 776 | 16.172 | 0.760 | −0.595 | 1.00 | 36.37 |
| ATOM | 1947 | OE1 | GLN | A | 776 | 15.258 | 1.315 | −1.209 | 1.00 | 39.80 |
| ATOM | 1948 | NE2 | GLN | A | 776 | 17.058 | −0.023 | −1.186 | 1.00 | 41.16 |
| ATOM | 1949 | C | GLN | A | 776 | 15.627 | 1.934 | 3.738 | 1.00 | 10.61 |
| ATOM | 1950 | O | GLN | A | 776 | 16.796 | 1.988 | 4.109 | 1.00 | 13.39 |
| ATOM | 1951 | N | ARG | A | 777 | 14.776 | 2.946 | 3.922 | 1.00 | 10.52 |
| ATOM | 1952 | CA | ARG | A | 777 | 15.199 | 4.197 | 4.534 | 1.00 | 11.43 |
| ATOM | 1953 | CB | ARG | A | 777 | 14.090 | 5.253 | 4.419 | 1.00 | 15.26 |
| ATOM | 1954 | CG | ARG | A | 777 | 14.487 | 6.607 | 4.948 | 1.00 | 20.06 |
| ATOM | 1955 | CD | ARG | A | 777 | 13.343 | 7.586 | 4.765 | 1.00 | 25.19 |
| ATOM | 1956 | NE | ARG | A | 777 | 12.206 | 7.273 | 5.627 | 1.00 | 34.37 |
| ATOM | 1957 | CZ | ARG | A | 777 | 12.232 | 7.359 | 6.954 | 1.00 | 35.62 |
| ATOM | 1958 | NH1 | ARG | A | 777 | 13.340 | 7.747 | 7.575 | 1.00 | 37.87 |
| ATOM | 1959 | NH2 | ARG | A | 777 | 11.149 | 7.068 | 7.661 | 1.00 | 29.66 |
| ATOM | 1960 | C | ARG | A | 777 | 15.560 | 3.979 | 5.997 | 1.00 | 10.83 |
| ATOM | 1961 | O | ARG | A | 777 | 16.601 | 4.447 | 6.472 | 1.00 | 13.15 |
| ATOM | 1962 | N | ILE | A | 778 | 14.715 | 3.238 | 6.705 | 1.00 | 12.38 |
| ATOM | 1963 | CA | ILE | A | 778 | 14.953 | 2.944 | 8.112 | 1.00 | 13.18 |
| ATOM | 1964 | CB | ILE | A | 778 | 13.753 | 2.209 | 8.744 | 1.00 | 15.50 |
| ATOM | 1965 | CG2 | ILE | A | 778 | 14.091 | 1.788 | 10.170 | 1.00 | 13.56 |
| ATOM | 1966 | CG1 | ILE | A | 778 | 12.516 | 3.108 | 8.721 | 1.00 | 22.29 |
| ATOM | 1967 | CD1 | ILE | A | 778 | 12.685 | 4.399 | 9.478 | 1.00 | 26.81 |
| ATOM | 1968 | C | ILE | A | 778 | 16.198 | 2.086 | 8.305 | 1.00 | 12.49 |
| ATOM | 1969 | O | ILE | A | 778 | 16.963 | 2.298 | 9.245 | 1.00 | 12.70 |
| ATOM | 1970 | N | ALA | A | 779 | 16.399 | 1.117 | 7.417 | 1.00 | 12.47 |
| ATOM | 1971 | CA | ALA | A | 779 | 17.563 | 0.242 | 7.510 | 1.00 | 13.19 |
| ATOM | 1972 | CB | ALA | A | 779 | 17.538 | −0.789 | 6.391 | 1.00 | 14.05 |
| ATOM | 1973 | C | ALA | A | 779 | 18.856 | 1.051 | 7.452 | 1.00 | 13.71 |
| ATOM | 1974 | O | ALA | A | 779 | 19.839 | 0.700 | 8.106 | 1.00 | 11.99 |
| ATOM | 1975 | N | GLU | A | 780 | 18.858 | 2.131 | 6.672 | 1.00 | 14.86 |
| ATOM | 1976 | CA | GLU | A | 780 | 20.048 | 2.976 | 6.560 | 1.00 | 13.62 |
| ATOM | 1977 | CB | GLU | A | 780 | 19.860 | 4.053 | 5.487 | 1.00 | 17.29 |
| ATOM | 1978 | CG | GLU | A | 780 | 19.742 | 3.519 | 4.075 | 1.00 | 30.45 |
| ATOM | 1979 | CD | GLU | A | 780 | 19.744 | 4.628 | 3.038 | 1.00 | 42.90 |
| ATOM | 1980 | OE1 | GLU | A | 780 | 20.761 | 5.347 | 2.939 | 1.00 | 42.08 |
| ATOM | 1981 | OE2 | GLU | A | 780 | 18.728 | 4.783 | 2.326 | 1.00 | 56.88 |
| ATOM | 1982 | C | GLU | A | 780 | 20.350 | 3.649 | 7.897 | 1.00 | 13.45 |
| ATOM | 1983 | O | GLU | A | 780 | 21.506 | 3.791 | 8.277 | 1.00 | 14.75 |
| ATOM | 1984 | N | LEU | A | 781 | 19.303 | 4.074 | 8.597 | 1.00 | 12.82 |
| ATOM | 1985 | CA | LEU | A | 781 | 19.464 | 4.716 | 9.898 | 1.00 | 10.66 |
| ATOM | 1986 | CB | LEU | A | 781 | 18.113 | 5.215 | 10.410 | 1.00 | 12.60 |
| ATOM | 1987 | CG | LEU | A | 781 | 17.428 | 6.296 | 9.574 | 1.00 | 15.49 |
| ATOM | 1988 | CD1 | LEU | A | 781 | 16.101 | 6.670 | 10.215 | 1.00 | 14.52 |
| ATOM | 1989 | CD2 | LEU | A | 781 | 18.339 | 7.513 | 9.475 | 1.00 | 17.00 |
| ATOM | 1990 | C | LEU | A | 781 | 20.032 | 3.704 | 10.887 | 1.00 | 12.39 |
| ATOM | 1991 | O | LEU | A | 781 | 20.935 | 4.014 | 11.673 | 1.00 | 11.98 |
| ATOM | 1992 | N | VAL | A | 782 | 19.490 | 2.490 | 10.839 | 1.00 | 9.90 |
| ATOM | 1993 | CA | VAL | A | 782 | 19.933 | 1.418 | 11.719 | 1.00 | 9.65 |
| ATOM | 1994 | CB | VAL | A | 782 | 19.116 | 0.126 | 11.463 | 1.00 | 10.42 |
| ATOM | 1995 | CG1 | VAL | A | 782 | 19.714 | −1.029 | 12.240 | 1.00 | 13.17 |
| ATOM | 1996 | CG2 | VAL | A | 782 | 17.657 | 0.339 | 11.868 | 1.00 | 16.63 |
| ATOM | 1997 | C | VAL | A | 782 | 21.418 | 1.154 | 11.477 | 1.00 | 10.70 |
| ATOM | 1998 | O | VAL | A | 782 | 22.210 | 1.068 | 12.418 | 1.00 | 13.37 |
| ATOM | 1999 | N | ALA | A | 783 | 21.793 | 1.053 | 10.206 | 1.00 | 11.25 |
| ATOM | 2000 | CA | ALA | A | 783 | 23.185 | 0.810 | 9.839 | 1.00 | 11.83 |
| ATOM | 2001 | CB | ALA | A | 783 | 23.299 | 0.632 | 8.337 | 1.00 | 15.10 |
| ATOM | 2002 | C | ALA | A | 783 | 24.074 | 1.961 | 10.298 | 1.00 | 12.03 |
| ATOM | 2003 | O | ALA | A | 783 | 25.170 | 1.746 | 10.820 | 1.00 | 12.81 |
| ATOM | 2004 | N | THR | A | 784 | 23.599 | 3.187 | 10.099 | 1.00 | 11.94 |
| ATOM | 2005 | CA | THR | A | 784 | 24.358 | 4.359 | 10.504 | 1.00 | 10.90 |
| ATOM | 2006 | CB | THR | A | 784 | 23.572 | 5.659 | 10.211 | 1.00 | 12.78 |
| ATOM | 2007 | OG1 | THR | A | 784 | 23.359 | 5.778 | 8.797 | 1.00 | 15.62 |
| ATOM | 2008 | CG2 | THR | A | 784 | 24.341 | 6.883 | 10.706 | 1.00 | 13.70 |
| ATOM | 2009 | C | THR | A | 784 | 24.676 | 4.282 | 11.995 | 1.00 | 11.74 |
| ATOM | 2010 | O | THR | A | 784 | 25.819 | 4.490 | 12.408 | 1.00 | 10.51 |
| ATOM | 2011 | N | GLU | A | 785 | 23.670 | 3.969 | 12.805 | 1.00 | 10.01 |
| ATOM | 2012 | CA | GLU | A | 785 | 23.881 | 3.880 | 14.243 | 1.00 | 10.75 |
| ATOM | 2013 | CB | GLU | A | 785 | 22.549 | 3.669 | 14.968 | 1.00 | 7.51 |
| ATOM | 2014 | CG | GLU | A | 785 | 22.675 | 3.740 | 16.483 | 1.00 | 9.65 |
| ATOM | 2015 | CD | GLU | A | 785 | 21.335 | 3.755 | 17.178 | 1.00 | 7.52 |
| ATOM | 2016 | OE1 | GLU | A | 785 | 20.415 | 4.436 | 16.674 | 1.00 | 10.93 |
| ATOM | 2017 | OE2 | GLU | A | 785 | 21.208 | 3.099 | 18.236 | 1.00 | 12.56 |
| ATOM | 2018 | C | GLU | A | 785 | 24.847 | 2.760 | 14.612 | 1.00 | 11.99 |
| ATOM | 2019 | O | GLU | A | 785 | 25.737 | 2.948 | 15.440 | 1.00 | 11.68 |
| ATOM | 2020 | N | PHE | A | 786 | 24.671 | 1.594 | 13.997 | 1.00 | 9.83 |
| ATOM | 2021 | CA | PHE | A | 786 | 25.541 | 0.464 | 14.285 | 1.00 | 10.39 |
| ATOM | 2022 | CB | PHE | A | 786 | 25.069 | −0.794 | 13.550 | 1.00 | 11.58 |
| ATOM | 2023 | CG | PHE | A | 786 | 23.876 | −1.455 | 14.176 | 1.00 | 16.59 |
| ATOM | 2024 | CD1 | PHE | A | 786 | 23.632 | −1.327 | 15.533 | 1.00 | 18.18 |
| ATOM | 2025 | CD2 | PHE | A | 786 | 23.034 | −2.254 | 13.419 | 1.00 | 19.90 |
| ATOM | 2026 | CE1 | PHE | A | 786 | 22.570 | −1.986 | 16.125 | 1.00 | 25.35 |
| ATOM | 2027 | CE2 | PHE | A | 786 | 21.972 | −2.916 | 14.004 | 1.00 | 17.12 |
| ATOM | 2028 | CZ | PHE | A | 786 | 21.740 | −2.781 | 15.360 | 1.00 | 16.84 |
| ATOM | 2029 | C | PHE | A | 786 | 26.985 | 0.760 | 13.898 | 1.00 | 9.62 |
| ATOM | 2030 | O | PHE | A | 786 | 27.907 | 0.444 | 14.652 | 1.00 | 10.46 |
| ATOM | 2031 | N | PHE | A | 787 | 27.183 | 1.348 | 12.720 | 1.00 | 8.90 |
| ATOM | 2032 | CA | PHE | A | 787 | 28.531 | 1.680 | 12.273 | 1.00 | 9.83 |
| ATOM | 2033 | CB | PHE | A | 787 | 28.515 | 2.141 | 10.809 | 1.00 | 10.96 |
| ATOM | 2034 | CG | PHE | A | 787 | 28.116 | 1.056 | 9.849 | 1.00 | 8.28 |
| ATOM | 2035 | CD1 | PHE | A | 787 | 28.335 | −0.276 | 10.172 | 1.00 | 10.72 |
| ATOM | 2036 | CD2 | PHE | A | 787 | 27.546 | 1.357 | 8.626 | 1.00 | 11.47 |
| ATOM | 2037 | CE1 | PHE | A | 787 | 27.993 | −1.287 | 9.296 | 1.00 | 12.45 |
| ATOM | 2038 | CE2 | PHE | A | 787 | 27.201 | 0.344 | 7.739 | 1.00 | 12.21 |
| ATOM | 2039 | CZ | PHE | A | 787 | 27.424 | −0.977 | 8.075 | 1.00 | 8.97 |
| ATOM | 2040 | C | PHE | A | 787 | 29.165 | 2.747 | 13.149 | 1.00 | 12.00 |
| ATOM | 2041 | O | PHE | A | 787 | 30.387 | 2.787 | 13.311 | 1.00 | 11.70 |
| ATOM | 2042 | N | ASP | A | 788 | 28.340 | 3.620 | 13.713 | 1.00 | 10.51 |
| ATOM | 2043 | CA | ASP | A | 788 | 28.867 | 4.655 | 14.583 | 1.00 | 13.07 |
| ATOM | 2044 | CB | ASP | A | 788 | 27.770 | 5.654 | 14.953 | 1.00 | 9.28 |
| ATOM | 2045 | CG | ASP | A | 788 | 28.275 | 6.751 | 15.869 | 1.00 | 17.65 |
| ATOM | 2046 | OD1 | ASP | A | 788 | 27.862 | 6.784 | 17.046 | 1.00 | 22.26 |
| ATOM | 2047 | OD2 | ASP | A | 788 | 29.097 | 7.575 | 15.414 | 1.00 | 23.70 |
| ATOM | 2048 | C | ASP | A | 788 | 29.427 | 3.975 | 15.832 | 1.00 | 11.61 |
| ATOM | 2049 | O | ASP | A | 788 | 30.491 | 4.349 | 16.324 | 1.00 | 11.23 |
| ATOM | 2050 | N | GLN | A | 789 | 28.719 | 2.965 | 16.338 | 1.00 | 10.39 |
| ATOM | 2051 | CA | GLN | A | 789 | 29.201 | 2.243 | 17.515 | 1.00 | 10.66 |
| ATOM | 2052 | CB | GLN | A | 789 | 28.185 | 1.199 | 17.979 | 1.00 | 14.04 |
| ATOM | 2053 | CG | GLN | A | 789 | 28.667 | 0.416 | 19.191 | 1.00 | 13.95 |
| ATOM | 2054 | CD | GLN | A | 789 | 27.696 | −0.652 | 19.641 | 1.00 | 13.88 |
| ATOM | 2055 | OE1 | GLN | A | 789 | 26.488 | −0.445 | 19.641 | 1.00 | 10.68 |
| ATOM | 2056 | NE2 | GLN | A | 789 | 28.226 | −1.799 | 20.051 | 1.00 | 10.88 |
| ATOM | 2057 | C | GLN | A | 789 | 30.509 | 1.541 | 17.155 | 1.00 | 12.25 |
| ATOM | 2058 | O | GLN | A | 789 | 31.452 | 1.504 | 17.952 | 1.00 | 12.92 |
| ATOM | 2059 | N | GLY | A | 790 | 30.552 | 0.979 | 15.950 | 1.00 | 12.27 |
| ATOM | 2060 | CA | GLY | A | 790 | 31.746 | 0.292 | 15.493 | 1.00 | 13.69 |
| ATOM | 2061 | C | GLY | A | 790 | 32.948 | 1.218 | 15.519 | 1.00 | 12.41 |
| ATOM | 2062 | O | GLY | A | 790 | 34.050 | 0.805 | 15.885 | 1.00 | 14.19 |
| ATOM | 2063 | N | ASP | A | 791 | 32.743 | 2.476 | 15.135 | 1.00 | 13.24 |
| ATOM | 2064 | CA | ASP | A | 791 | 33.837 | 3.448 | 15.135 | 1.00 | 15.89 |
| ATOM | 2065 | CB | ASP | A | 791 | 33.383 | 4.802 | 14.582 | 1.00 | 11.78 |
| ATOM | 2066 | CG | ASP | A | 791 | 33.022 | 4.748 | 13.108 | 1.00 | 11.72 |
| ATOM | 2067 | OD1 | ASP | A | 791 | 33.525 | 3.853 | 12.401 | 1.00 | 16.07 |
| ATOM | 2068 | OD2 | ASP | A | 791 | 32.249 | 5.619 | 12.657 | 1.00 | 16.56 |
| ATOM | 2069 | C | ASP | A | 791 | 34.350 | 3.653 | 16.554 | 1.00 | 15.06 |
| ATOM | 2070 | O | ASP | A | 791 | 35.555 | 3.751 | 16.794 | 1.00 | 15.97 |
| ATOM | 2071 | N | ARG | A | 792 | 33.423 | 3.723 | 17.496 | 1.00 | 16.00 |
| ATOM | 2072 | CA | ARG | A | 792 | 33.783 | 3.925 | 18.885 | 1.00 | 13.38 |
| ATOM | 2073 | CB | ARG | A | 792 | 32.544 | 4.308 | 19.680 | 1.00 | 15.67 |
| ATOM | 2074 | CG | ARG | A | 792 | 32.056 | 5.689 | 19.304 | 1.00 | 20.98 |
| ATOM | 2075 | CD | ARG | A | 792 | 30.791 | 6.043 | 20.014 | 1.00 | 22.55 |
| ATOM | 2076 | NE | ARG | A | 792 | 30.929 | 6.004 | 21.465 | 1.00 | 21.96 |
| ATOM | 2077 | CZ | ARG | A | 792 | 29.945 | 6.332 | 22.290 | 1.00 | 19.00 |
| ATOM | 2078 | NH1 | ARG | A | 792 | 28.782 | 6.726 | 21.791 | 1.00 | 18.95 |
| ATOM | 2079 | NH2 | ARG | A | 792 | 30.105 | 6.236 | 23.601 | 1.00 | 23.88 |
| ATOM | 2080 | C | ARG | A | 792 | 34.466 | 2.716 | 19.501 | 1.00 | 13.13 |
| ATOM | 2081 | O | ARG | A | 792 | 35.296 | 2.865 | 20.390 | 1.00 | 14.33 |
| ATOM | 2082 | N | GLU | A | 793 | 34.112 | 1.522 | 19.036 | 1.00 | 14.11 |
| ATOM | 2083 | CA | GLU | A | 793 | 34.730 | 0.305 | 19.545 | 1.00 | 12.55 |
| ATOM | 2084 | CB | GLU | A | 793 | 33.969 | −0.932 | 19.051 | 1.00 | 12.93 |
| ATOM | 2085 | CG | GLU | A | 793 | 32.531 | −0.986 | 19.543 | 1.00 | 15.09 |
| ATOM | 2086 | CD | GLU | A | 793 | 31.816 | −2.269 | 19.165 | 1.00 | 17.10 |
| ATOM | 2087 | OE1 | GLU | A | 793 | 32.105 | −2.807 | 18.078 | 1.00 | 14.75 |
| ATOM | 2088 | OE2 | GLU | A | 793 | 30.951 | −2.728 | 19.949 | 1.00 | 13.37 |
| ATOM | 2089 | C | GLU | A | 793 | 36.184 | 0.251 | 19.080 | 1.00 | 13.70 |
| ATOM | 2090 | O | GLU | A | 793 | 37.073 | −0.143 | 19.837 | 1.00 | 14.48 |
| ATOM | 2091 | N | ARG | A | 794 | 36.429 | 0.655 | 17.838 | 1.00 | 12.88 |
| ATOM | 2092 | CA | ARG | A | 794 | 37.792 | 0.647 | 17.318 | 1.00 | 16.50 |
| ATOM | 2093 | CB | ARG | A | 794 | 37.828 | 1.083 | 15.847 | 1.00 | 16.91 |
| ATOM | 2094 | CG | ARG | A | 794 | 37.330 | 0.047 | 14.838 | 1.00 | 16.40 |
| ATOM | 2095 | CD | ARG | A | 794 | 37.399 | 0.609 | 13.422 | 1.00 | 20.95 |
| ATOM | 2096 | NE | ARG | A | 794 | 36.790 | −0.268 | 12.422 | 1.00 | 21.25 |
| ATOM | 2097 | CZ | ARG | A | 794 | 37.323 | −1.407 | 11.996 | 1.00 | 16.41 |
| ATOM | 2098 | NH1 | ARG | A | 794 | 38.486 | −1.822 | 12.480 | 1.00 | 26.91 |
| ATOM | 2099 | NH2 | ARG | A | 794 | 36.691 | −2.134 | 11.082 | 1.00 | 18.30 |
| ATOM | 2100 | C | ARG | A | 794 | 38.648 | 1.603 | 18.141 | 1.00 | 17.48 |
| ATOM | 2101 | O | ARG | A | 794 | 39.781 | 1.287 | 18.503 | 1.00 | 18.61 |
| ATOM | 2102 | N | LYS | A | 795 | 38.087 | 2.769 | 18.443 | 1.00 | 19.00 |
| ATOM | 2103 | CA | LYS | A | 795 | 38.793 | 3.795 | 19.199 | 1.00 | 19.03 |
| ATOM | 2104 | CB | LYS | A | 795 | 38.146 | 5.161 | 18.947 | 1.00 | 22.77 |
| ATOM | 2105 | CG | LYS | A | 795 | 38.839 | 6.312 | 19.661 | 1.00 | 30.54 |
| ATOM | 2106 | CD | LYS | A | 795 | 38.128 | 7.633 | 19.410 | 1.00 | 38.45 |
| ATOM | 2107 | CE | LYS | A | 795 | 38.902 | 8.796 | 20.009 | 1.00 | 34.57 |
| ATOM | 2108 | NZ | LYS | A | 795 | 40.267 | 8.886 | 19.424 | 1.00 | 42.21 |
| ATOM | 2109 | C | LYS | A | 795 | 38.866 | 3.554 | 20.704 | 1.00 | 19.86 |
| ATOM | 2110 | O | LYS | A | 795 | 39.923 | 3.716 | 21.310 | 1.00 | 21.68 |
| ATOM | 2111 | N | GLU | A | 796 | 37.747 | 3.166 | 21.304 | 1.00 | 17.50 |
| ATOM | 2112 | CA | GLU | A | 796 | 37.696 | 2.953 | 22.745 | 1.00 | 18.20 |
| ATOM | 2113 | CB | GLU | A | 796 | 36.286 | 3.272 | 23.255 | 1.00 | 16.63 |
| ATOM | 2114 | CG | GLU | A | 796 | 35.872 | 4.722 | 23.048 | 1.00 | 19.55 |
| ATOM | 2115 | CD | GLU | A | 796 | 34.425 | 4.982 | 23.414 | 1.00 | 17.22 |
| ATOM | 2116 | OE1 | GLU | A | 796 | 33.986 | 4.520 | 24.489 | 1.00 | 17.11 |
| ATOM | 2117 | OE2 | GLU | A | 796 | 33.729 | 5.660 | 22.629 | 1.00 | 17.31 |
| ATOM | 2118 | C | GLU | A | 796 | 38.116 | 1.568 | 23.238 | 1.00 | 18.82 |
| ATOM | 2119 | O | GLU | A | 796 | 38.604 | 1.432 | 24.359 | 1.00 | 19.88 |
| ATOM | 2120 | N | LEU | A | 797 | 37.936 | 0.544 | 22.408 | 1.00 | 17.76 |
| ATOM | 2121 | CA | LEU | A | 797 | 38.279 | −0.817 | 22.809 | 1.00 | 17.55 |
| ATOM | 2122 | CB | LEU | A | 797 | 37.032 | −1.708 | 22.732 | 1.00 | 14.21 |
| ATOM | 2123 | CG | LEU | A | 797 | 35.816 | −1.230 | 23.535 | 1.00 | 21.04 |
| ATOM | 2124 | CD1 | LEU | A | 797 | 34.655 | −2.194 | 23.333 | 1.00 | 19.81 |
| ATOM | 2125 | CD2 | LEU | A | 797 | 36.177 | −1.134 | 25.008 | 1.00 | 22.47 |
| ATOM | 2126 | C | LEU | A | 797 | 39.400 | −1.441 | 21.980 | 1.00 | 18.06 |
| ATOM | 2127 | O | LEU | A | 797 | 39.899 | −2.518 | 22.312 | 1.00 | 18.10 |
| ATOM | 2128 | N | ASN | A | 798 | 39.795 | −0.760 | 20.910 | 1.00 | 17.87 |
| ATOM | 2129 | CA | ASN | A | 798 | 40.846 | −1.244 | 20.016 | 1.00 | 17.14 |
| ATOM | 2130 | CB | ASN | A | 798 | 42.202 | −1.278 | 20.732 | 1.00 | 18.53 |
| ATOM | 2131 | CG | ASN | A | 798 | 43.357 | −1.501 | 19.770 | 1.00 | 19.84 |
| ATOM | 2132 | OD1 | ASN | A | 798 | 43.497 | −0.774 | 18.788 | 1.00 | 22.65 |
| ATOM | 2133 | ND2 | ASN | A | 798 | 44.186 | −2.507 | 20.043 | 1.00 | 17.46 |
| ATOM | 2134 | C | ASN | A | 798 | 40.522 | −2.632 | 19.472 | 1.00 | 19.40 |
| ATOM | 2135 | O | ASN | A | 798 | 41.400 | −3.485 | 19.352 | 1.00 | 18.37 |
| ATOM | 2136 | N | ILE | A | 799 | 39.254 | −2.852 | 19.141 | 1.00 | 18.23 |
| ATOM | 2137 | CA | ILE | A | 799 | 38.816 | −4.134 | 18.603 | 1.00 | 20.72 |
| ATOM | 2138 | CB | ILE | A | 799 | 37.779 | −4.811 | 19.529 | 1.00 | 23.69 |
| ATOM | 2139 | CG2 | ILE | A | 799 | 38.348 | −4.970 | 20.930 | 1.00 | 25.12 |
| ATOM | 2140 | CG1 | ILE | A | 799 | 36.500 | −3.968 | 19.570 | 1.00 | 27.29 |
| ATOM | 2141 | CD1 | ILE | A | 799 | 35.381 | −4.581 | 20.373 | 1.00 | 23.09 |
| ATOM | 2142 | C | ILE | A | 799 | 38.158 | −3.926 | 17.243 | 1.00 | 20.66 |
| ATOM | 2143 | O | ILE | A | 799 | 37.636 | −2.846 | 16.956 | 1.00 | 20.48 |
| ATOM | 2144 | N | GLU | A | 800 | 38.199 | −4.951 | 16.399 | 1.00 | 20.84 |
| ATOM | 2145 | CA | GLU | A | 800 | 37.558 | −4.855 | 15.096 | 1.00 | 19.43 |
| ATOM | 2146 | CB | GLU | A | 800 | 38.162 | −5.856 | 14.109 | 1.00 | 25.89 |
| ATOM | 2147 | CG | GLU | A | 800 | 37.507 | −5.813 | 12.735 | 1.00 | 37.89 |
| ATOM | 2148 | CD | GLU | A | 800 | 38.245 | −6.637 | 11.699 | 1.00 | 48.08 |
| ATOM | 2149 | OE1 | GLU | A | 800 | 38.421 | −7.856 | 11.915 | 1.00 | 47.23 |
| ATOM | 2150 | OE2 | GLU | A | 800 | 38.647 | −6.063 | 10.663 | 1.00 | 52.27 |
| ATOM | 2151 | C | GLU | A | 800 | 36.095 | −5.188 | 15.345 | 1.00 | 16.86 |
| ATOM | 2152 | O | GLU | A | 800 | 35.774 | −6.249 | 15.879 | 1.00 | 20.00 |
| ATOM | 2153 | N | PRO | A | 801 | 35.186 | −4.277 | 14.979 | 1.00 | 16.72 |
| ATOM | 2154 | CD | PRO | A | 801 | 35.413 | −2.971 | 14.340 | 1.00 | 13.94 |
| ATOM | 2155 | CA | PRO | A | 801 | 33.755 | −4.518 | 15.188 | 1.00 | 15.17 |
| ATOM | 2156 | CB | PRO | A | 801 | 33.101 | −3.242 | 14.651 | 1.00 | 11.35 |
| ATOM | 2157 | CG | PRO | A | 801 | 34.189 | −2.204 | 14.760 | 1.00 | 18.39 |
| ATOM | 2158 | C | PRO | A | 801 | 33.249 | −5.750 | 14.446 | 1.00 | 14.88 |
| ATOM | 2159 | O | PRO | A | 801 | 33.836 | −6.168 | 13.437 | 1.00 | 14.57 |
| ATOM | 2160 | N | THR | A | 802 | 32.166 | −6.333 | 14.952 | 1.00 | 13.89 |
| ATOM | 2161 | CA | THR | A | 802 | 31.554 | −7.473 | 14.282 | 1.00 | 13.70 |
| ATOM | 2162 | CB | THR | A | 802 | 30.367 | −8.057 | 15.090 | 1.00 | 18.66 |
| ATOM | 2163 | OG1 | THR | A | 802 | 29.339 | −7.067 | 15.227 | 1.00 | 22.82 |
| ATOM | 2164 | CG2 | THR | A | 802 | 30.824 | −8.491 | 16.475 | 1.00 | 28.52 |
| ATOM | 2165 | C | THR | A | 802 | 31.028 | −6.886 | 12.974 | 1.00 | 13.06 |
| ATOM | 2166 | O | THR | A | 802 | 30.896 | −5.666 | 12.853 | 1.00 | 13.12 |
| ATOM | 2167 | N | ASP | A | 803 | 30.730 | −7.737 | 11.997 | 1.00 | 13.68 |
| ATOM | 2168 | CA | ASP | A | 803 | 30.239 | −7.260 | 10.706 | 1.00 | 13.10 |
| ATOM | 2169 | CB | ASP | A | 803 | 29.784 | −8.436 | 9.840 | 1.00 | 15.94 |
| ATOM | 2170 | CG | ASP | A | 803 | 30.942 | −9.281 | 9.340 | 1.00 | 19.70 |
| ATOM | 2171 | OD1 | ASP | A | 803 | 32.108 | −8.965 | 9.661 | 1.00 | 16.86 |
| ATOM | 2172 | OD2 | ASP | A | 803 | 30.681 | −10.265 | 8.619 | 1.00 | 17.57 |
| ATOM | 2173 | C | ASP | A | 803 | 29.092 | −6.262 | 10.829 | 1.00 | 14.26 |
| ATOM | 2174 | O | ASP | A | 803 | 29.071 | −5.239 | 10.141 | 1.00 | 15.59 |
| ATOM | 2175 | N | LEU | A | 804 | 28.142 | −6.561 | 11.706 | 1.00 | 12.76 |
| ATOM | 2176 | CA | LEU | A | 804 | 26.982 | −5.700 | 11.907 | 1.00 | 13.21 |
| ATOM | 2177 | CB | LEU | A | 804 | 26.116 | −6.251 | 13.042 | 1.00 | 14.65 |
| ATOM | 2178 | CG | LEU | A | 804 | 24.861 | −5.430 | 13.347 | 1.00 | 15.24 |
| ATOM | 2179 | CD1 | LEU | A | 804 | 24.020 | −5.317 | 12.092 | 1.00 | 16.29 |
| ATOM | 2180 | CD2 | LEU | A | 804 | 24.068 | −6.089 | 14.461 | 1.00 | 23.77 |
| ATOM | 2181 | C | LEU | A | 804 | 27.356 | −4.252 | 12.214 | 1.00 | 13.48 |
| ATOM | 2182 | O | LEU | A | 804 | 26.646 | −3.317 | 11.825 | 1.00 | 13.22 |
| ATOM | 2183 | N | MET | A | 805 | 28.476 | −4.067 | 12.904 | 1.00 | 12.28 |
| ATOM | 2184 | CA | MET | A | 805 | 28.909 | −2.725 | 13.276 | 1.00 | 11.75 |
| ATOM | 2185 | CB | MET | A | 805 | 29.141 | −2.671 | 14.790 | 1.00 | 13.28 |
| ATOM | 2186 | CG | MET | A | 805 | 27.859 | −2.865 | 15.589 | 1.00 | 33.42 |
| ATOM | 2187 | SD | MET | A | 805 | 28.125 | −3.166 | 17.338 | 1.00 | 47.91 |
| ATOM | 2188 | CE | MET | A | 805 | 26.447 | −3.244 | 17.925 | 1.00 | 28.62 |
| ATOM | 2189 | C | MET | A | 805 | 30.160 | −2.268 | 12.542 | 1.00 | 11.31 |
| ATOM | 2190 | O | MET | A | 805 | 30.761 | −1.259 | 12.908 | 1.00 | 12.42 |
| ATOM | 2191 | N | ASN | A | 806 | 30.530 | −2.992 | 11.490 | 1.00 | 11.94 |
| ATOM | 2192 | CA | ASN | A | 806 | 31.726 | −2.673 | 10.712 | 1.00 | 13.21 |
| ATOM | 2193 | CB | ASN | A | 806 | 32.537 | −3.954 | 10.471 | 1.00 | 13.42 |
| ATOM | 2194 | CG | ASN | A | 806 | 33.907 | −3.676 | 9.876 | 1.00 | 16.11 |
| ATOM | 2195 | OD1 | ASN | A | 806 | 34.209 | −2.549 | 9.484 | 1.00 | 17.92 |
| ATOM | 2196 | ND2 | ASN | A | 806 | 34.740 | −4.706 | 9.802 | 1.00 | 15.54 |
| ATOM | 2197 | C | ASN | A | 806 | 31.371 | −2.023 | 9.374 | 1.00 | 14.20 |
| ATOM | 2198 | O | ASN | A | 806 | 30.988 | −2.715 | 8.430 | 1.00 | 14.08 |
| ATOM | 2199 | N | ARG | A | 807 | 31.508 | −0.699 | 9.296 | 1.00 | 12.56 |
| ATOM | 2200 | CA | ARG | A | 807 | 31.189 | 0.037 | 8.075 | 1.00 | 14.91 |
| ATOM | 2201 | CB | ARG | A | 807 | 31.410 | 1.543 | 8.275 | 1.00 | 16.99 |
| ATOM | 2202 | CG | ARG | A | 807 | 30.891 | 2.385 | 7.116 | 1.00 | 25.83 |
| ATOM | 2203 | CD | ARG | A | 807 | 31.162 | 3.881 | 7.280 | 1.00 | 30.30 |
| ATOM | 2204 | NE | ARG | A | 807 | 30.520 | 4.470 | 8.457 | 1.00 | 20.02 |
| ATOM | 2205 | CZ | ARG | A | 807 | 31.078 | 4.525 | 9.662 | 1.00 | 16.27 |
| ATOM | 2206 | NH1 | ARG | A | 807 | 32.290 | 4.025 | 9.854 | 1.00 | 20.90 |
| ATOM | 2207 | NH2 | ARG | A | 807 | 30.431 | 5.095 | 10.670 | 1.00 | 15.75 |
| ATOM | 2208 | C | ARG | A | 807 | 32.021 | −0.442 | 6.889 | 1.00 | 13.89 |
| ATOM | 2209 | O | ARG | A | 807 | 31.573 | −0.392 | 5.736 | 1.00 | 13.84 |
| ATOM | 2210 | N | GLU | A | 808 | 33.228 | −0.910 | 7.177 | 1.00 | 11.68 |
| ATOM | 2211 | CA | GLU | A | 808 | 34.122 | −1.397 | 6.137 | 1.00 | 17.06 |
| ATOM | 2212 | CB | GLU | A | 808 | 35.502 | −1.675 | 6.731 | 1.00 | 16.87 |
| ATOM | 2213 | CG | GLU | A | 808 | 36.256 | −0.394 | 7.085 | 1.00 | 22.64 |
| ATOM | 2214 | CD | GLU | A | 808 | 37.556 | −0.645 | 7.819 | 1.00 | 24.85 |
| ATOM | 2215 | OE1 | GLU | A | 808 | 38.293 | −1.576 | 7.430 | 1.00 | 26.32 |
| ATOM | 2216 | OE2 | GLU | A | 808 | 37.846 | 0.100 | 8.780 | 1.00 | 36.15 |
| ATOM | 2217 | C | GLU | A | 808 | 33.565 | −2.641 | 5.461 | 1.00 | 15.58 |
| ATOM | 2218 | O | GLU | A | 808 | 33.929 | −2.956 | 4.329 | 1.00 | 15.69 |
| ATOM | 2219 | N | LYS | A | 809 | 32.677 | −3.343 | 6.156 | 1.00 | 14.84 |
| ATOM | 2220 | CA | LYS | A | 809 | 32.062 | −4.544 | 5.600 | 1.00 | 16.67 |
| ATOM | 2221 | CB | LYS | A | 809 | 32.381 | −5.765 | 6.478 | 1.00 | 12.30 |
| ATOM | 2222 | CG | LYS | A | 809 | 33.845 | −6.184 | 6.405 | 1.00 | 13.87 |
| ATOM | 2223 | CD | LYS | A | 809 | 34.151 | −7.408 | 7.259 | 1.00 | 14.82 |
| ATOM | 2224 | CE | LYS | A | 809 | 33.430 | −8.641 | 6.745 | 1.00 | 25.64 |
| ATOM | 2225 | NZ | LYS | A | 809 | 33.789 | −9.854 | 7.534 | 1.00 | 21.95 |
| ATOM | 2226 | C | LYS | A | 809 | 30.555 | −4.363 | 5.445 | 1.00 | 15.95 |
| ATOM | 2227 | O | LYS | A | 809 | 29.770 | −5.281 | 5.693 | 1.00 | 18.05 |
| ATOM | 2228 | N | LYS | A | 810 | 30.156 | −3.167 | 5.022 | 1.00 | 15.13 |
| ATOM | 2229 | CA | LYS | A | 810 | 28.742 | −2.867 | 4.828 | 1.00 | 16.24 |
| ATOM | 2230 | CB | LYS | A | 810 | 28.549 | −1.383 | 4.494 | 1.00 | 14.35 |
| ATOM | 2231 | CG | LYS | A | 810 | 29.347 | −0.869 | 3.312 | 1.00 | 24.48 |
| ATOM | 2232 | CD | LYS | A | 810 | 29.222 | 0.647 | 3.226 | 1.00 | 29.64 |
| ATOM | 2233 | CE | LYS | A | 810 | 29.994 | 1.224 | 2.049 | 1.00 | 35.68 |
| ATOM | 2234 | NZ | LYS | A | 810 | 29.432 | 0.786 | 0.746 | 1.00 | 42.39 |
| ATOM | 2235 | C | LYS | A | 810 | 28.139 | −3.744 | 3.734 | 1.00 | 14.42 |
| ATOM | 2236 | O | LYS | A | 810 | 26.921 | −3.843 | 3.607 | 1.00 | 14.44 |
| ATOM | 2237 | N | ASN | A | 811 | 28.995 | −4.389 | 2.950 | 1.00 | 13.62 |
| ATOM | 2238 | CA | ASN | A | 811 | 28.511 | −5.270 | 1.893 | 1.00 | 13.60 |
| ATOM | 2239 | CB | ASN | A | 811 | 29.680 | −5.746 | 1.016 | 1.00 | 18.14 |
| ATOM | 2240 | CG | ASN | A | 811 | 30.815 | −6.340 | 1.826 | 1.00 | 21.65 |
| ATOM | 2241 | OD1 | ASN | A | 811 | 30.936 | −7.559 | 1.958 | 1.00 | 17.54 |
| ATOM | 2242 | ND2 | ASN | A | 811 | 31.651 | −5.472 | 2.388 | 1.00 | 13.93 |
| ATOM | 2243 | C | ASN | A | 811 | 27.777 | −6.474 | 2.487 | 1.00 | 14.60 |
| ATOM | 2244 | O | ASN | A | 811 | 27.084 | −7.197 | 1.777 | 1.00 | 13.25 |
| ATOM | 2245 | N | LYS | A | 812 | 27.922 | −6.677 | 3.796 | 1.00 | 14.05 |
| ATOM | 2246 | CA | LYS | A | 812 | 27.279 | −7.797 | 4.478 | 1.00 | 15.17 |
| ATOM | 2247 | CB | LYS | A | 812 | 28.057 | −8.150 | 5.753 | 1.00 | 15.23 |
| ATOM | 2248 | CG | LYS | A | 812 | 29.401 | −8.803 | 5.500 | 1.00 | 20.27 |
| ATOM | 2249 | CD | LYS | A | 812 | 29.198 | −10.137 | 4.817 | 1.00 | 28.44 |
| ATOM | 2250 | CE | LYS | A | 812 | 30.497 | −10.886 | 4.658 | 1.00 | 35.44 |
| ATOM | 2251 | NZ | LYS | A | 812 | 30.257 | −12.223 | 4.048 | 1.00 | 38.51 |
| ATOM | 2252 | C | LYS | A | 812 | 25.818 | −7.542 | 4.859 | 1.00 | 15.78 |
| ATOM | 2253 | O | LYS | A | 812 | 25.109 | −8.475 | 5.258 | 1.00 | 13.98 |
| ATOM | 2254 | N | ILE | A | 813 | 25.366 | −6.297 | 4.724 | 1.00 | 14.64 |
| ATOM | 2255 | CA | ILE | A | 813 | 24.012 | −5.964 | 5.144 | 1.00 | 14.83 |
| ATOM | 2256 | CB | ILE | A | 813 | 23.770 | −4.460 | 5.007 | 1.00 | 15.21 |
| ATOM | 2257 | CG2 | ILE | A | 813 | 22.264 | −4.158 | 5.091 | 1.00 | 15.81 |
| ATOM | 2258 | CG1 | ILE | A | 813 | 24.616 | −3.740 | 6.073 | 1.00 | 13.99 |
| ATOM | 2259 | CD1 | ILE | A | 813 | 24.445 | −2.265 | 6.070 | 1.00 | 27.43 |
| ATOM | 2260 | C | ILE | A | 813 | 22.862 | −6.792 | 4.546 | 1.00 | 14.67 |
| ATOM | 2261 | O | ILE | A | 813 | 22.042 | −7.309 | 5.270 | 1.00 | 13.44 |
| ATOM | 2262 | N | PRO | A | 814 | 22.807 | −6.936 | 3.224 | 1.00 | 12.07 |
| ATOM | 2263 | CD | PRO | A | 814 | 23.603 | −6.274 | 2.191 | 1.00 | 14.86 |
| ATOM | 2264 | CA | PRO | A | 814 | 21.709 | −7.724 | 2.623 | 1.00 | 14.39 |
| ATOM | 2265 | CB | PRO | A | 814 | 22.060 | −7.714 | 1.152 | 1.00 | 13.45 |
| ATOM | 2266 | CG | PRO | A | 814 | 22.693 | −6.351 | 1.001 | 1.00 | 14.67 |
| ATOM | 2267 | C | PRO | A | 814 | 21.605 | −9.144 | 3.185 | 1.00 | 11.82 |
| ATOM | 2268 | O | PRO | A | 814 | 20.525 | −9.603 | 3.570 | 1.00 | 12.87 |
| ATOM | 2269 | N | SER | A | 815 | 22.730 | −9.846 | 3.215 | 1.00 | 11.71 |
| ATOM | 2270 | CA | SER | A | 815 | 22.730 | −11.205 | 3.729 | 1.00 | 13.13 |
| ATOM | 2271 | CB | SER | A | 815 | 24.102 | −11.863 | 3.526 | 1.00 | 14.66 |
| ATOM | 2272 | OG | SER | A | 815 | 25.124 | −11.185 | 4.233 | 1.00 | 27.24 |
| ATOM | 2273 | C | SER | A | 815 | 22.366 | −11.220 | 5.212 | 1.00 | 14.09 |
| ATOM | 2274 | O | SER | A | 815 | 21.677 | −12.120 | 5.682 | 1.00 | 14.61 |
| ATOM | 2275 | N | MET | A | 816 | 22.809 | −10.211 | 5.937 | 1.00 | 14.82 |
| ATOM | 2276 | CA | MET | A | 816 | 22.498 | −10.197 | 7.353 | 1.00 | 14.08 |
| ATOM | 2277 | CB | MET | A | 816 | 23.449 | −9.254 | 8.079 | 1.00 | 16.95 |
| ATOM | 2278 | CG | MET | A | 816 | 24.829 | −9.872 | 8.232 | 1.00 | 19.08 |
| ATOM | 2279 | SD | MET | A | 816 | 26.019 | −8.905 | 9.168 | 1.00 | 23.16 |
| ATOM | 2280 | CE | MET | A | 816 | 25.502 | −9.310 | 10.812 | 1.00 | 29.41 |
| ATOM | 2281 | C | MET | A | 816 | 21.057 | −9.838 | 7.628 | 1.00 | 12.78 |
| ATOM | 2282 | O | MET | A | 816 | 20.477 | −10.281 | 8.613 | 1.00 | 13.06 |
| ATOM | 2283 | N | GLN | A | 817 | 20.464 | −9.010 | 6.765 | 1.00 | 11.62 |
| ATOM | 2284 | CA | GLN | A | 817 | 19.061 | −8.637 | 6.915 | 1.00 | 11.33 |
| ATOM | 2285 | CB | GLN | A | 817 | 18.666 | −7.547 | 5.899 | 1.00 | 12.94 |
| ATOM | 2286 | CG | GLN | A | 817 | 19.186 | −6.179 | 6.283 | 1.00 | 11.95 |
| ATOM | 2287 | CD | GLN | A | 817 | 18.469 | −5.624 | 7.500 | 1.00 | 17.94 |
| ATOM | 2288 | OE1 | GLN | A | 817 | 18.544 | −6.183 | 8.595 | 1.00 | 34.56 |
| ATOM | 2289 | NE2 | GLN | A | 817 | 17.759 | −4.523 | 7.309 | 1.00 | 34.13 |
| ATOM | 2290 | C | GLN | A | 817 | 18.202 | −9.882 | 6.714 | 1.00 | 13.31 |
| ATOM | 2291 | O | GLN | A | 817 | 17.254 | −10.116 | 7.472 | 1.00 | 10.14 |
| ATOM | 2292 | N | VAL | A | 818 | 18.537 | −10.698 | 5.715 | 1.00 | 12.18 |
| ATOM | 2293 | CA | VAL | A | 818 | 17.774 | −11.918 | 5.479 | 1.00 | 11.53 |
| ATOM | 2294 | CB | VAL | A | 818 | 18.252 | −12.659 | 4.210 | 1.00 | 13.56 |
| ATOM | 2295 | CG1 | VAL | A | 818 | 17.500 | −13.972 | 4.063 | 1.00 | 10.58 |
| ATOM | 2296 | CG2 | VAL | A | 818 | 18.032 | −11.781 | 2.982 | 1.00 | 17.92 |
| ATOM | 2297 | C | VAL | A | 818 | 17.903 | −12.858 | 6.678 | 1.00 | 11.51 |
| ATOM | 2298 | O | VAL | A | 818 | 16.930 | −13.494 | 7.090 | 1.00 | 12.55 |
| ATOM | 2299 | N | GLY | A | 819 | 19.110 | −12.934 | 7.231 | 1.00 | 11.44 |
| ATOM | 2300 | CA | GLY | A | 819 | 19.356 | −13.791 | 8.376 | 1.00 | 12.74 |
| ATOM | 2301 | C | GLY | A | 819 | 18.556 | −13.335 | 9.579 | 1.00 | 13.96 |
| ATOM | 2302 | O | GLY | A | 819 | 18.068 | −14.149 | 10.360 | 1.00 | 13.49 |
| ATOM | 2303 | N | PHE | A | 820 | 18.430 | −12.022 | 9.725 | 1.00 | 14.20 |
| ATOM | 2304 | CA | PHE | A | 820 | 17.682 | −11.426 | 10.825 | 1.00 | 11.70 |
| ATOM | 2305 | CB | PHE | A | 820 | 17.927 | −9.913 | 10.833 | 1.00 | 11.32 |
| ATOM | 2306 | CG | PHE | A | 820 | 17.122 | −9.167 | 11.858 | 1.00 | 9.99 |
| ATOM | 2307 | CD1 | PHE | A | 820 | 17.268 | −9.436 | 13.209 | 1.00 | 23.52 |
| ATOM | 2308 | CD2 | PHE | A | 820 | 16.225 | −8.184 | 11.466 | 1.00 | 17.26 |
| ATOM | 2309 | CE1 | PHE | A | 820 | 16.529 | −8.740 | 14.151 | 1.00 | 19.87 |
| ATOM | 2310 | CE2 | PHE | A | 820 | 15.485 | −7.488 | 12.403 | 1.00 | 16.59 |
| ATOM | 2311 | CZ | PHE | A | 820 | 15.640 | −7.767 | 13.746 | 1.00 | 17.66 |
| ATOM | 2312 | C | PHE | A | 820 | 16.191 | −11.731 | 10.651 | 1.00 | 12.70 |
| ATOM | 2313 | O | PHE | A | 820 | 15.498 | −12.103 | 11.604 | 1.00 | 10.72 |
| ATOM | 2314 | N | ILE | A | 821 | 15.697 | −11.571 | 9.429 | 1.00 | 10.04 |
| ATOM | 2315 | CA | ILE | A | 821 | 14.298 | −11.850 | 9.150 | 1.00 | 9.29 |
| ATOM | 2316 | CB | ILE | A | 821 | 13.957 | −11.540 | 7.679 | 1.00 | 10.14 |
| ATOM | 2317 | CG2 | ILE | A | 821 | 12.617 | −12.166 | 7.307 | 1.00 | 12.87 |
| ATOM | 2318 | CG1 | ILE | A | 821 | 13.931 | −10.022 | 7.479 | 1.00 | 12.03 |
| ATOM | 2319 | CD1 | ILE | A | 821 | 13.795 | −9.580 | 6.037 | 1.00 | 14.77 |
| ATOM | 2320 | C | ILE | A | 821 | 13.991 | −13.311 | 9.462 | 1.00 | 11.67 |
| ATOM | 2321 | O | ILE | A | 821 | 13.012 | −13.617 | 10.140 | 1.00 | 11.27 |
| ATOM | 2322 | N | ASP | A | 822 | 14.841 | −14.215 | 8.985 | 1.00 | 11.97 |
| ATOM | 2323 | CA | ASP | A | 822 | 14.634 | −15.637 | 9.237 | 1.00 | 14.30 |
| ATOM | 2324 | CB | ASP | A | 822 | 15.630 | −16.479 | 8.428 | 1.00 | 17.59 |
| ATOM | 2325 | CG | ASP | A | 822 | 15.317 | −16.499 | 6.942 | 1.00 | 17.92 |
| ATOM | 2326 | OD1 | ASP | A | 822 | 14.177 | −16.153 | 6.564 | 1.00 | 15.98 |
| ATOM | 2327 | OD2 | ASP | A | 822 | 16.212 | −16.880 | 6.152 | 1.00 | 13.97 |
| ATOM | 2328 | C | ASP | A | 822 | 14.759 | −16.021 | 10.714 | 1.00 | 15.42 |
| ATOM | 2329 | O | ASP | A | 822 | 13.876 | −16.671 | 11.275 | 1.00 | 16.53 |
| ATOM | 2330 | N | ALA | A | 823 | 15.849 | −15.608 | 11.345 | 1.00 | 13.55 |
| ATOM | 2331 | CA | ALA | A | 823 | 16.101 | −15.971 | 12.737 | 1.00 | 15.03 |
| ATOM | 2332 | CB | ALA | A | 823 | 17.580 | −15.756 | 13.058 | 1.00 | 13.84 |
| ATOM | 2333 | C | ALA | A | 823 | 15.254 | −15.299 | 13.810 | 1.00 | 15.13 |
| ATOM | 2334 | O | ALA | A | 823 | 14.907 | −15.928 | 14.810 | 1.00 | 17.91 |
| ATOM | 2335 | N | ILE | A | 824 | 14.914 | −14.032 | 13.606 | 1.00 | 13.91 |
| ATOM | 2336 | CA | ILE | A | 824 | 14.169 | −13.281 | 14.611 | 1.00 | 14.87 |
| ATOM | 2337 | CB | ILE | A | 824 | 14.929 | −11.971 | 14.966 | 1.00 | 16.51 |
| ATOM | 2338 | CG2 | ILE | A | 824 | 14.085 | −11.106 | 15.891 | 1.00 | 11.92 |
| ATOM | 2339 | CG1 | ILE | A | 824 | 16.291 | −12.293 | 15.589 | 1.00 | 19.03 |
| ATOM | 2340 | CD1 | ILE | A | 824 | 16.211 | −12.966 | 16.938 | 1.00 | 17.79 |
| ATOM | 2341 | C | ILE | A | 824 | 12.736 | −12.872 | 14.271 | 1.00 | 15.38 |
| ATOM | 2342 | O | ILE | A | 824 | 11.817 | −13.051 | 15.077 | 1.00 | 15.58 |
| ATOM | 2343 | N | CYS | A | 825 | 12.548 | −12.332 | 13.074 | 1.00 | 11.66 |
| ATOM | 2344 | CA | CYS | A | 825 | 11.248 | −11.798 | 12.682 | 1.00 | 10.83 |
| ATOM | 2345 | CB | CYS | A | 825 | 11.470 | −10.717 | 11.629 | 1.00 | 14.06 |
| ATOM | 2346 | SG | CYS | A | 825 | 12.684 | −9.498 | 12.156 | 1.00 | 12.72 |
| ATOM | 2347 | C | CYS | A | 825 | 10.094 | −12.685 | 12.231 | 1.00 | 12.83 |
| ATOM | 2348 | O | CYS | A | 825 | 9.003 | −12.606 | 12.792 | 1.00 | 11.78 |
| ATOM | 2349 | N | LEU | A | 826 | 10.312 | −13.508 | 11.213 | 1.00 | 10.64 |
| ATOM | 2350 | CA | LEU | A | 826 | 9.230 | −14.343 | 10.699 | 1.00 | 12.29 |
| ATOM | 2351 | CB | LEU | A | 826 | 9.759 | −15.291 | 9.621 | 1.00 | 17.45 |
| ATOM | 2352 | CG | LEU | A | 826 | 10.132 | −14.585 | 8.314 | 1.00 | 14.44 |
| ATOM | 2353 | CD1 | LEU | A | 826 | 10.756 | −15.585 | 7.355 | 1.00 | 19.69 |
| ATOM | 2354 | CD2 | LEU | A | 826 | 8.884 | −13.965 | 7.693 | 1.00 | 17.13 |
| ATOM | 2355 | C | LEU | A | 826 | 8.428 | −15.131 | 11.735 | 1.00 | 11.75 |
| ATOM | 2356 | O | LEU | A | 826 | 7.194 | −15.094 | 11.728 | 1.00 | 12.87 |
| ATOM | 2357 | N | GLN | A | 827 | 9.115 | −15.842 | 12.620 | 1.00 | 9.56 |
| ATOM | 2358 | CA | GLN | A | 827 | 8.426 | −16.637 | 13.632 | 1.00 | 10.96 |
| ATOM | 2359 | CB | GLN | A | 827 | 9.438 | −17.368 | 14.507 | 1.00 | 9.95 |
| ATOM | 2360 | CG | GLN | A | 827 | 10.207 | −18.465 | 13.805 | 1.00 | 14.60 |
| ATOM | 2361 | CD | GLN | A | 827 | 11.365 | −18.949 | 14.646 | 1.00 | 27.21 |
| ATOM | 2362 | OE1 | GLN | A | 827 | 12.420 | −18.312 | 14.695 | 1.00 | 20.68 |
| ATOM | 2363 | NE2 | GLN | A | 827 | 11.169 | −20.068 | 15.335 | 1.00 | 24.19 |
| ATOM | 2364 | C | GLN | A | 827 | 7.516 | −15.798 | 14.524 | 1.00 | 13.14 |
| ATOM | 2365 | O | GLN | A | 827 | 6.428 | −16.235 | 14.906 | 1.00 | 13.21 |
| ATOM | 2366 | N | LEU | A | 828 | 7.966 | −14.596 | 14.862 | 1.00 | 10.02 |
| ATOM | 2367 | CA | LEU | A | 828 | 7.182 | −13.713 | 15.720 | 1.00 | 10.78 |
| ATOM | 2368 | CB | LEU | A | 828 | 8.037 | −12.521 | 16.166 | 1.00 | 9.67 |
| ATOM | 2369 | CG | LEU | A | 828 | 7.315 | −11.431 | 16.966 | 1.00 | 14.41 |
| ATOM | 2370 | CD1 | LEU | A | 828 | 6.653 | −12.048 | 18.185 | 1.00 | 16.13 |
| ATOM | 2371 | CD2 | LEU | A | 828 | 8.300 | −10.351 | 17.389 | 1.00 | 14.03 |
| ATOM | 2372 | C | LEU | A | 828 | 5.902 | −13.218 | 15.036 | 1.00 | 11.55 |
| ATOM | 2373 | O | LEU | A | 828 | 4.814 | −13.290 | 15.609 | 1.00 | 12.50 |
| ATOM | 2374 | N | TYR | A | 829 | 6.024 | −12.717 | 13.812 | 1.00 | 10.38 |
| ATOM | 2375 | CA | TYR | A | 829 | 4.851 | −12.217 | 13.109 | 1.00 | 11.38 |
| ATOM | 2376 | CB | TYR | A | 829 | 5.286 | −11.392 | 11.892 | 1.00 | 9.07 |
| ATOM | 2377 | CG | TYR | A | 829 | 5.967 | −10.112 | 12.324 | 1.00 | 13.32 |
| ATOM | 2378 | CD1 | TYR | A | 829 | 5.298 | −9.189 | 13.118 | 1.00 | 8.62 |
| ATOM | 2379 | CE1 | TYR | A | 829 | 5.926 | −8.037 | 13.569 | 1.00 | 10.58 |
| ATOM | 2380 | CD2 | TYR | A | 829 | 7.290 | −9.849 | 11.989 | 1.00 | 10.78 |
| ATOM | 2381 | CE2 | TYR | A | 829 | 7.930 | −8.699 | 12.440 | 1.00 | 8.02 |
| ATOM | 2382 | CZ | TYR | A | 829 | 7.241 | −7.800 | 13.228 | 1.00 | 8.64 |
| ATOM | 2383 | OH | TYR | A | 829 | 7.867 | −6.659 | 13.688 | 1.00 | 10.63 |
| ATOM | 2384 | C | TYR | A | 829 | 3.908 | −13.349 | 12.731 | 1.00 | 12.07 |
| ATOM | 2385 | O | TYR | A | 829 | 2.704 | −13.140 | 12.594 | 1.00 | 14.07 |
| ATOM | 2386 | N | GLU | A | 830 | 4.452 | −14.555 | 12.585 | 1.00 | 14.19 |
| ATOM | 2387 | CA | GLU | A | 830 | 3.614 | −15.709 | 12.274 | 1.00 | 14.53 |
| ATOM | 2388 | CB | GLU | A | 830 | 4.469 | −16.919 | 11.881 | 1.00 | 15.21 |
| ATOM | 2389 | CG | GLU | A | 830 | 5.196 | −16.751 | 10.557 | 1.00 | 18.48 |
| ATOM | 2390 | CD | GLU | A | 830 | 6.057 | −17.948 | 10.208 | 1.00 | 27.35 |
| ATOM | 2391 | OE1 | GLU | A | 830 | 6.422 | −18.701 | 11.133 | 1.00 | 25.55 |
| ATOM | 2392 | OE2 | GLU | A | 830 | 6.380 | −18.127 | 9.014 | 1.00 | 25.64 |
| ATOM | 2393 | C | GLU | A | 830 | 2.806 | −16.027 | 13.531 | 1.00 | 15.34 |
| ATOM | 2394 | O | GLU | A | 830 | 1.594 | −16.245 | 13.462 | 1.00 | 15.05 |
| ATOM | 2395 | N | ALA | A | 831 | 3.484 | −16.037 | 14.679 | 1.00 | 12.88 |
| ATOM | 2396 | CA | ALA | A | 831 | 2.836 | −16.319 | 15.958 | 1.00 | 13.60 |
| ATOM | 2397 | CB | ALA | A | 831 | 3.877 | −16.341 | 17.080 | 1.00 | 14.75 |
| ATOM | 2398 | C | ALA | A | 831 | 1.764 | −15.279 | 16.264 | 1.00 | 14.36 |
| ATOM | 2399 | O | ALA | A | 831 | 0.666 | −15.617 | 16.714 | 1.00 | 13.70 |
| ATOM | 2400 | N | LEU | A | 832 | 2.085 | −14.010 | 16.026 | 1.00 | 11.70 |
| ATOM | 2401 | CA | LEU | A | 832 | 1.135 | −12.935 | 16.276 | 1.00 | 12.90 |
| ATOM | 2402 | CB | LEU | A | 832 | 1.800 | −11.577 | 16.030 | 1.00 | 12.02 |
| ATOM | 2403 | CG | LEU | A | 832 | 0.935 | −10.334 | 16.256 | 1.00 | 13.07 |
| ATOM | 2404 | CD1 | LEU | A | 832 | 0.367 | −10.354 | 17.670 | 1.00 | 16.97 |
| ATOM | 2405 | CD2 | LEU | A | 832 | 1.764 | −9.076 | 16.018 | 1.00 | 12.70 |
| ATOM | 2406 | C | LEU | A | 832 | −0.093 | −13.098 | 15.382 | 1.00 | 13.07 |
| ATOM | 2407 | O | LEU | A | 832 | −1.221 | −12.851 | 15.811 | 1.00 | 12.63 |
| ATOM | 2408 | N | THR | A | 833 | 0.125 | −13.522 | 14.140 | 1.00 | 10.33 |
| ATOM | 2409 | CA | THR | A | 833 | −0.985 | −13.720 | 13.213 | 1.00 | 11.65 |
| ATOM | 2410 | CB | THR | A | 833 | −0.471 | −14.019 | 11.798 | 1.00 | 15.54 |
| ATOM | 2411 | OG1 | THR | A | 833 | 0.250 | −12.881 | 11.309 | 1.00 | 13.44 |
| ATOM | 2412 | CG2 | THR | A | 833 | −1.631 | −14.319 | 10.856 | 1.00 | 20.09 |
| ATOM | 2413 | C | THR | A | 833 | −1.856 | −14.875 | 13.709 | 1.00 | 13.24 |
| ATOM | 2414 | O | THR | A | 833 | −3.070 | −14.879 | 13.512 | 1.00 | 14.62 |
| ATOM | 2415 | N | HIS | A | 834 | −1.233 | −15.855 | 14.358 | 1.00 | 12.04 |
| ATOM | 2416 | CA | HIS | A | 834 | −1.974 | −16.988 | 14.901 | 1.00 | 15.35 |
| ATOM | 2417 | CB | HIS | A | 834 | −1.010 | −18.045 | 15.451 | 1.00 | 17.31 |
| ATOM | 2418 | CG | HIS | A | 834 | −1.686 | −19.161 | 16.174 | 1.00 | 21.70 |
| ATOM | 2419 | CD2 | HIS | A | 834 | −2.005 | −20.422 | 15.792 | 1.00 | 27.35 |
| ATOM | 2420 | ND1 | HIS | A | 834 | −2.158 | −19.040 | 17.471 | 1.00 | 27.23 |
| ATOM | 2421 | CE1 | HIS | A | 834 | −2.728 | −20.162 | 17.843 | 1.00 | 22.73 |
| ATOM | 2422 | NE2 | HIS | A | 834 | −2.650 | −21.026 | 16.836 | 1.00 | 26.03 |
| ATOM | 2423 | C | HIS | A | 834 | −2.907 | −16.495 | 16.011 | 1.00 | 16.43 |
| ATOM | 2424 | O | HIS | A | 834 | −4.061 | −16.917 | 16.101 | 1.00 | 16.32 |
| ATOM | 2425 | N | VAL | A | 835 | −2.402 | −15.600 | 16.852 | 1.00 | 14.39 |
| ATOM | 2426 | CA | VAL | A | 835 | −3.201 | −15.042 | 17.940 | 1.00 | 16.17 |
| ATOM | 2427 | CB | VAL | A | 835 | −2.329 | −14.177 | 18.879 | 1.00 | 20.06 |
| ATOM | 2428 | CG1 | VAL | A | 835 | −3.203 | −13.433 | 19.878 | 1.00 | 26.18 |
| ATOM | 2429 | CG2 | VAL | A | 835 | −1.336 | −15.067 | 19.616 | 1.00 | 22.85 |
| ATOM | 2430 | C | VAL | A | 835 | −4.345 | −14.196 | 17.376 | 1.00 | 18.30 |
| ATOM | 2431 | O | VAL | A | 835 | −5.462 | −14.219 | 17.899 | 1.00 | 16.68 |
| ATOM | 2432 | N | SER | A | 836 | −4.061 | −13.461 | 16.302 | 1.00 | 15.55 |
| ATOM | 2433 | CA | SER | A | 836 | −5.060 | −12.620 | 15.646 | 1.00 | 15.62 |
| ATOM | 2434 | CB | SER | A | 836 | −5.081 | −11.222 | 16.267 | 1.00 | 18.44 |
| ATOM | 2435 | OG | SER | A | 836 | −6.092 | −10.430 | 15.665 | 1.00 | 24.05 |
| ATOM | 2436 | C | SER | A | 836 | −4.744 | −12.516 | 14.153 | 1.00 | 15.03 |
| ATOM | 2437 | O | SER | A | 836 | −3.754 | −11.901 | 13.759 | 1.00 | 14.82 |
| ATOM | 2438 | N | GLU | A | 837 | −5.596 | −13.123 | 13.334 | 1.00 | 16.29 |
| ATOM | 2439 | CA | GLU | A | 837 | −5.423 | −13.139 | 11.886 | 1.00 | 16.40 |
| ATOM | 2440 | CB | GLU | A | 837 | −6.578 | −13.922 | 11.244 | 1.00 | 19.29 |
| ATOM | 2441 | CG | GLU | A | 837 | −6.524 | −14.017 | 9.722 | 1.00 | 30.56 |
| ATOM | 2442 | CD | GLU | A | 837 | −5.259 | −14.685 | 9.213 | 1.00 | 40.78 |
| ATOM | 2443 | OE1 | GLU | A | 837 | −4.925 | −15.785 | 9.704 | 1.00 | 33.63 |
| ATOM | 2444 | OE2 | GLU | A | 837 | −4.603 | −14.112 | 8.316 | 1.00 | 45.55 |
| ATOM | 2445 | C | GLU | A | 837 | −5.324 | −11.749 | 11.256 | 1.00 | 14.00 |
| ATOM | 2446 | O | GLU | A | 837 | −4.672 | −11.574 | 10.227 | 1.00 | 16.56 |
| ATOM | 2447 | N | ASP | A | 838 | −5.967 | −10.762 | 11.871 | 1.00 | 14.38 |
| ATOM | 2448 | CA | ASP | A | 838 | −5.938 | −9.402 | 11.349 | 1.00 | 12.78 |
| ATOM | 2449 | CB | ASP | A | 838 | −6.990 | −8.547 | 12.060 | 1.00 | 12.80 |
| ATOM | 2450 | CG | ASP | A | 838 | −8.410 | −8.929 | 11.669 | 1.00 | 31.66 |
| ATOM | 2451 | OD1 | ASP | A | 838 | −8.770 | −8.750 | 10.487 | 1.00 | 25.15 |
| ATOM | 2452 | OD2 | ASP | A | 838 | −9.162 | −9.414 | 12.540 | 1.00 | 29.94 |
| ATOM | 2453 | C | ASP | A | 838 | −4.558 | −8.751 | 11.468 | 1.00 | 11.81 |
| ATOM | 2454 | O | ASP | A | 838 | −4.355 | −7.625 | 11.017 | 1.00 | 12.87 |
| ATOM | 2455 | N | CYS | A | 839 | −3.610 | −9.456 | 12.080 | 1.00 | 14.34 |
| ATOM | 2456 | CA | CYS | A | 839 | −2.254 | −8.929 | 12.203 | 1.00 | 14.23 |
| ATOM | 2457 | CB | CYS | A | 839 | −1.619 | −9.354 | 13.533 | 1.00 | 12.27 |
| ATOM | 2458 | SG | CYS | A | 839 | −2.115 | −8.349 | 14.969 | 1.00 | 12.53 |
| ATOM | 2459 | C | CYS | A | 839 | −1.412 | −9.440 | 11.038 | 1.00 | 15.74 |
| ATOM | 2460 | O | CYS | A | 839 | −0.222 | −9.142 | 10.941 | 1.00 | 13.52 |
| ATOM | 2461 | N | PHE | A | 840 | −2.044 | −10.209 | 10.152 | 1.00 | 14.15 |
| ATOM | 2462 | CA | PHE | A | 840 | −1.364 | −10.765 | 8.983 | 1.00 | 14.85 |
| ATOM | 2463 | CB | PHE | A | 840 | −2.370 | −11.451 | 8.052 | 1.00 | 18.01 |
| ATOM | 2464 | CG | PHE | A | 840 | −1.760 | −11.926 | 6.764 | 1.00 | 16.92 |
| ATOM | 2465 | CD1 | PHE | A | 840 | −0.886 | −13.001 | 6.751 | 1.00 | 13.52 |
| ATOM | 2466 | CD2 | PHE | A | 840 | −2.010 | −11.257 | 5.575 | 1.00 | 19.38 |
| ATOM | 2467 | CE1 | PHE | A | 840 | −0.269 | −13.398 | 5.575 | 1.00 | 23.30 |
| ATOM | 2468 | CE2 | PHE | A | 840 | −1.397 | −11.649 | 4.396 | 1.00 | 23.75 |
| ATOM | 2469 | CZ | PHE | A | 840 | −0.525 | −12.720 | 4.397 | 1.00 | 22.76 |
| ATOM | 2470 | C | PHE | A | 840 | −0.541 | −9.768 | 8.155 | 1.00 | 13.70 |
| ATOM | 2471 | O | PHE | A | 840 | 0.546 | −10.105 | 7.678 | 1.00 | 13.33 |
| ATOM | 2472 | N | PRO | A | 841 | −1.049 | −8.537 | 7.958 | 1.00 | 12.97 |
| ATOM | 2473 | CD | PRO | A | 841 | −2.369 | −8.002 | 8.339 | 1.00 | 14.47 |
| ATOM | 2474 | CA | PRO | A | 841 | −0.294 | −7.557 | 7.168 | 1.00 | 12.84 |
| ATOM | 2475 | CB | PRO | A | 841 | −1.127 | −6.287 | 7.312 | 1.00 | 14.37 |
| ATOM | 2476 | CG | PRO | A | 841 | −2.533 | −6.826 | 7.391 | 1.00 | 14.58 |
| ATOM | 2477 | C | PRO | A | 841 | 1.151 | −7.367 | 7.630 | 1.00 | 14.54 |
| ATOM | 2478 | O | PRO | A | 841 | 2.034 | −7.065 | 6.826 | 1.00 | 13.75 |
| ATOM | 2479 | N | LEU | A | 842 | 1.393 | −7.530 | 8.926 | 1.00 | 11.66 |
| ATOM | 2480 | CA | LEU | A | 842 | 2.747 | −7.384 | 9.444 | 1.00 | 12.35 |
| ATOM | 2481 | CB | LEU | A | 842 | 2.732 | −7.430 | 10.977 | 1.00 | 11.85 |
| ATOM | 2482 | CG | LEU | A | 842 | 2.039 | −6.228 | 11.624 | 1.00 | 12.78 |
| ATOM | 2483 | CD1 | LEU | A | 842 | 1.900 | −6.434 | 13.131 | 1.00 | 14.23 |
| ATOM | 2484 | CD2 | LEU | A | 842 | 2.837 | −4.975 | 11.321 | 1.00 | 14.25 |
| ATOM | 2485 | C | LEU | A | 842 | 3.613 | −8.513 | 8.880 | 1.00 | 13.95 |
| ATOM | 2486 | O | LEU | A | 842 | 4.741 | −8.290 | 8.444 | 1.00 | 13.84 |
| ATOM | 2487 | N | LEU | A | 843 | 3.067 | −9.727 | 8.882 | 1.00 | 11.90 |
| ATOM | 2488 | CA | LEU | A | 843 | 3.779 | −10.889 | 8.359 | 1.00 | 11.64 |
| ATOM | 2489 | CB | LEU | A | 843 | 2.995 | −12.161 | 8.670 | 1.00 | 10.07 |
| ATOM | 2490 | CG | LEU | A | 843 | 3.532 | −13.468 | 8.084 | 1.00 | 9.50 |
| ATOM | 2491 | CD1 | LEU | A | 843 | 4.940 | −13.735 | 8.588 | 1.00 | 13.64 |
| ATOM | 2492 | CD2 | LEU | A | 843 | 2.597 | −14.608 | 8.466 | 1.00 | 12.37 |
| ATOM | 2493 | C | LEU | A | 843 | 3.971 | −10.751 | 6.847 | 1.00 | 11.74 |
| ATOM | 2494 | O | LEU | A | 843 | 5.054 | −11.003 | 6.319 | 1.00 | 13.14 |
| ATOM | 2495 | N | ASP | A | 844 | 2.909 | −10.342 | 6.160 | 1.00 | 13.31 |
| ATOM | 2496 | CA | ASP | A | 844 | 2.943 | −10.154 | 4.711 | 1.00 | 14.45 |
| ATOM | 2497 | CB | ASP | A | 844 | 1.573 | −9.639 | 4.245 | 1.00 | 18.19 |
| ATOM | 2498 | CG | ASP | A | 844 | 1.354 | −9.771 | 2.739 | 1.00 | 13.51 |
| ATOM | 2499 | OD1 | ASP | A | 844 | 2.141 | −10.459 | 2.057 | 1.00 | 18.86 |
| ATOM | 2500 | OD2 | ASP | A | 844 | 0.367 | −9.187 | 2.243 | 1.00 | 17.91 |
| ATOM | 2501 | C | ASP | A | 844 | 4.053 | −9.153 | 4.366 | 1.00 | 13.87 |
| ATOM | 2502 | O | ASP | A | 844 | 4.854 | −9.386 | 3.460 | 1.00 | 13.07 |
| ATOM | 2503 | N | GLY | A | 845 | 4.100 | −8.046 | 5.104 | 1.00 | 12.67 |
| ATOM | 2504 | CA | GLY | A | 845 | 5.115 | −7.035 | 4.862 | 1.00 | 11.69 |
| ATOM | 2505 | C | GLY | A | 845 | 6.529 | −7.540 | 5.097 | 1.00 | 11.05 |
| ATOM | 2506 | O | GLY | A | 845 | 7.447 | −7.240 | 4.330 | 1.00 | 12.51 |
| ATOM | 2507 | N | CYS | A | 846 | 6.704 | −8.314 | 6.161 | 1.00 | 10.28 |
| ATOM | 2508 | CA | CYS | A | 846 | 8.005 | −8.882 | 6.507 | 1.00 | 10.93 |
| ATOM | 2509 | CB | CYS | A | 846 | 7.889 | −9.643 | 7.831 | 1.00 | 14.58 |
| ATOM | 2510 | SG | CYS | A | 846 | 9.443 | −10.273 | 8.485 | 1.00 | 12.82 |
| ATOM | 2511 | C | CYS | A | 846 | 8.497 | −9.821 | 5.398 | 1.00 | 11.92 |
| ATOM | 2512 | O | CYS | A | 846 | 9.661 | −9.762 | 4.985 | 1.00 | 11.24 |
| ATOM | 2513 | N | ARG | A | 847 | 7.605 | −10.683 | 4.914 | 1.00 | 12.77 |
| ATOM | 2514 | CA | ARG | A | 847 | 7.953 | −11.618 | 3.853 | 1.00 | 13.72 |
| ATOM | 2515 | CB | ARG | A | 847 | 6.762 | −12.527 | 3.535 | 1.00 | 11.63 |
| ATOM | 2516 | CG | ARG | A | 847 | 6.457 | −13.545 | 4.619 | 1.00 | 13.10 |
| ATOM | 2517 | CD | ARG | A | 847 | 5.180 | −14.308 | 4.316 | 1.00 | 17.35 |
| ATOM | 2518 | NE | ARG | A | 847 | 4.978 | −15.402 | 5.262 | 1.00 | 19.23 |
| ATOM | 2519 | CZ | ARG | A | 847 | 3.840 | −16.077 | 5.389 | 1.00 | 21.47 |
| ATOM | 2520 | NH1 | ARG | A | 847 | 2.796 | −15.769 | 4.632 | 1.00 | 24.29 |
| ATOM | 2521 | NH2 | ARG | A | 847 | 3.750 | −17.059 | 6.275 | 1.00 | 27.21 |
| ATOM | 2522 | C | ARG | A | 847 | 8.371 | −10.870 | 2.592 | 1.00 | 13.53 |
| ATOM | 2523 | O | ARG | A | 847 | 9.320 | −11.265 | 1.906 | 1.00 | 13.20 |
| ATOM | 2524 | N | LYS | A | 848 | 7.659 | −9.793 | 2.283 | 1.00 | 12.35 |
| ATOM | 2525 | CA | LYS | A | 848 | 7.977 | −9.007 | 1.099 | 1.00 | 14.65 |
| ATOM | 2526 | CB | LYS | A | 848 | 6.925 | −7.915 | 0.880 | 1.00 | 16.22 |
| ATOM | 2527 | CG | LYS | A | 848 | 5.567 | −8.447 | 0.413 | 1.00 | 14.77 |
| ATOM | 2528 | CD | LYS | A | 848 | 4.579 | −7.308 | 0.195 | 1.00 | 19.65 |
| ATOM | 2529 | CE | LYS | A | 848 | 3.210 | −7.817 | −0.206 | 1.00 | 19.42 |
| ATOM | 2530 | NZ | LYS | A | 848 | 2.214 | −6.709 | −0.212 | 1.00 | 23.11 |
| ATOM | 2531 | C | LYS | A | 848 | 9.364 | −8.392 | 1.229 | 1.00 | 14.80 |
| ATOM | 2532 | O | LYS | A | 848 | 10.112 | −8.319 | 0.253 | 1.00 | 13.12 |
| ATOM | 2533 | N | ASN | A | 849 | 9.713 | −7.953 | 2.435 | 1.00 | 13.42 |
| ATOM | 2534 | CA | ASN | A | 849 | 11.034 | −7.369 | 2.648 | 1.00 | 13.07 |
| ATOM | 2535 | CB | ASN | A | 849 | 11.086 | −6.646 | 3.995 | 1.00 | 13.67 |
| ATOM | 2536 | CG | ASN | A | 849 | 10.412 | −5.288 | 3.950 | 1.00 | 17.85 |
| ATOM | 2537 | OD1 | ASN | A | 849 | 9.863 | −4.821 | 4.949 | 1.00 | 16.87 |
| ATOM | 2538 | ND2 | ASN | A | 849 | 10.461 | −4.638 | 2.790 | 1.00 | 13.21 |
| ATOM | 2539 | C | ASN | A | 849 | 12.122 | −8.434 | 2.568 | 1.00 | 12.00 |
| ATOM | 2540 | O | ASN | A | 849 | 13.252 | −8.148 | 2.165 | 1.00 | 11.88 |
| ATOM | 2541 | N | ARG | A | 850 | 11.797 | −9.667 | 2.945 | 1.00 | 11.53 |
| ATOM | 2542 | CA | ARG | A | 850 | 12.793 | −10.729 | 2.860 | 1.00 | 11.98 |
| ATOM | 2543 | CB | ARG | A | 850 | 12.285 | −12.037 | 3.473 | 1.00 | 14.13 |
| ATOM | 2544 | CG | ARG | A | 850 | 13.373 | −13.110 | 3.508 | 1.00 | 13.55 |
| ATOM | 2545 | CD | ARG | A | 850 | 12.838 | −14.511 | 3.781 | 1.00 | 18.02 |
| ATOM | 2546 | NE | ARG | A | 850 | 13.930 | −15.457 | 4.001 | 1.00 | 16.95 |
| ATOM | 2547 | CZ | ARG | A | 850 | 14.743 | −15.919 | 3.052 | 1.00 | 14.40 |
| ATOM | 2548 | NH1 | ARG | A | 850 | 14.595 | −15.533 | 1.790 | 1.00 | 16.03 |
| ATOM | 2549 | NH2 | ARG | A | 850 | 15.727 | −16.754 | 3.373 | 1.00 | 12.51 |
| ATOM | 2550 | C | ARG | A | 850 | 13.138 | −10.972 | 1.399 | 1.00 | 13.81 |
| ATOM | 2551 | O | ARG | A | 850 | 14.304 | −11.151 | 1.054 | 1.00 | 15.14 |
| ATOM | 2552 | N | GLN | A | 851 | 12.118 | −10.985 | 0.542 | 1.00 | 16.03 |
| ATOM | 2553 | CA | GLN | A | 851 | 12.317 | −11.193 | −0.892 | 1.00 | 15.42 |
| ATOM | 2554 | CB | GLN | A | 851 | 10.969 | −11.156 | −1.622 | 1.00 | 17.30 |
| ATOM | 2555 | CG | GLN | A | 851 | 11.039 | −11.399 | −3.127 | 1.00 | 30.46 |
| ATOM | 2556 | CD | GLN | A | 851 | 11.475 | −10.174 | −3.912 | 1.00 | 34.81 |
| ATOM | 2557 | OE1 | GLN | A | 851 | 10.862 | −9.110 | −3.814 | 1.00 | 37.41 |
| ATOM | 2558 | NE2 | GLN | A | 851 | 12.534 | −10.320 | −4.704 | 1.00 | 40.89 |
| ATOM | 2559 | C | GLN | A | 851 | 13.229 | −10.109 | −1.436 | 1.00 | 16.49 |
| ATOM | 2560 | O | GLN | A | 851 | 14.151 | −10.371 | −2.208 | 1.00 | 15.70 |
| ATOM | 2561 | N | LYS | A | 852 | 12.953 | −8.867 | −1.014 | 1.00 | 15.86 |
| ATOM | 2562 | CA | LYS | A | 852 | 13.705 | −7.685 | −1.428 | 1.00 | 16.63 |
| ATOM | 2563 | CB | LYS | A | 852 | 13.065 | −6.405 | −0.838 | 1.00 | 13.58 |
| ATOM | 2564 | CG | LYS | A | 852 | 11.802 | −6.008 | −1.561 | 1.00 | 18.37 |
| ATOM | 2565 | CD | LYS | A | 852 | 12.183 | −5.582 | −2.958 | 1.00 | 31.95 |
| ATOM | 2566 | CE | LYS | A | 852 | 11.002 | −5.087 | −3.751 | 1.00 | 37.83 |
| ATOM | 2567 | NZ | LYS | A | 852 | 11.421 | −4.599 | −5.099 | 1.00 | 41.92 |
| ATOM | 2568 | C | LYS | A | 852 | 15.175 | −7.754 | −1.037 | 1.00 | 15.46 |
| ATOM | 2569 | O | LYS | A | 852 | 16.055 | −7.542 | −1.874 | 1.00 | 14.58 |
| ATOM | 2570 | N | TRP | A | 853 | 15.446 | −8.063 | 0.225 | 1.00 | 12.99 |
| ATOM | 2571 | CA | TRP | A | 853 | 16.821 | −8.168 | 0.705 | 1.00 | 12.72 |
| ATOM | 2572 | CB | TRP | A | 853 | 16.843 | −8.299 | 2.228 | 1.00 | 11.94 |
| ATOM | 2573 | CG | TRP | A | 853 | 16.640 | −7.011 | 2.957 | 1.00 | 11.40 |
| ATOM | 2574 | CD2 | TRP | A | 853 | 17.485 | −5.864 | 2.913 | 1.00 | 14.74 |
| ATOM | 2575 | CE2 | TRP | A | 853 | 16.929 | −4.888 | 3.763 | 1.00 | 12.41 |
| ATOM | 2576 | CE3 | TRP | A | 853 | 18.675 | −5.554 | 2.236 | 1.00 | 10.90 |
| ATOM | 2577 | CD1 | TRP | A | 853 | 15.618 | −6.701 | 3.813 | 1.00 | 11.02 |
| ATOM | 2578 | NE1 | TRP | A | 853 | 15.781 | −5.429 | 4.303 | 1.00 | 14.15 |
| ATOM | 2579 | CZ2 | TRP | A | 853 | 17.502 | −3.638 | 3.958 | 1.00 | 12.03 |
| ATOM | 2580 | CZ3 | TRP | A | 853 | 19.246 | −4.312 | 2.427 | 1.00 | 13.77 |
| ATOM | 2581 | CH2 | TRP | A | 853 | 18.663 | −3.370 | 3.280 | 1.00 | 16.08 |
| ATOM | 2582 | C | TRP | A | 853 | 17.543 | −9.357 | 0.074 | 1.00 | 13.55 |
| ATOM | 2583 | O | TRP | A | 853 | 18.722 | −9.265 | −0.282 | 1.00 | 13.19 |
| ATOM | 2584 | N | GLN | A | 854 | 16.836 | −10.472 | −0.062 | 1.00 | 12.06 |
| ATOM | 2585 | CA | GLN | A | 854 | 17.427 | −11.667 | −0.657 | 1.00 | 13.49 |
| ATOM | 2586 | CB | GLN | A | 854 | 16.426 | −12.822 | −0.630 | 1.00 | 13.86 |
| ATOM | 2587 | CG | GLN | A | 854 | 16.934 | −14.128 | −1.242 | 1.00 | 15.91 |
| ATOM | 2588 | CD | GLN | A | 854 | 18.267 | −14.585 | −0.665 | 1.00 | 23.66 |
| ATOM | 2589 | OE1 | GLN | A | 854 | 18.544 | −14.404 | 0.520 | 1.00 | 20.44 |
| ATOM | 2590 | NE2 | GLN | A | 854 | 19.091 | −15.199 | −1.504 | 1.00 | 26.84 |
| ATOM | 2591 | C | GLN | A | 854 | 17.869 | −11.400 | −2.094 | 1.00 | 13.14 |
| ATOM | 2592 | O | GLN | A | 854 | 18.920 | −11.880 | −2.524 | 1.00 | 15.55 |
| ATOM | 2593 | N | ALA | A | 855 | 17.069 | −10.635 | −2.832 | 1.00 | 14.63 |
| ATOM | 2594 | CA | ALA | A | 855 | 17.408 | −10.318 | −4.219 | 1.00 | 15.08 |
| ATOM | 2595 | CB | ALA | A | 855 | 16.264 | −9.563 | −4.884 | 1.00 | 17.06 |
| ATOM | 2596 | C | ALA | A | 855 | 18.697 | −9.504 | −4.303 | 1.00 | 16.23 |
| ATOM | 2597 | O | ALA | A | 855 | 19.439 | −9.611 | −5.278 | 1.00 | 17.06 |
| ATOM | 2598 | N | LEU | A | 856 | 18.959 | −8.689 | −3.287 | 1.00 | 15.11 |
| ATOM | 2599 | CA | LEU | A | 856 | 20.178 | −7.889 | −3.261 | 1.00 | 17.23 |
| ATOM | 2600 | CB | LEU | A | 856 | 20.031 | −6.715 | −2.284 | 1.00 | 11.53 |
| ATOM | 2601 | CG | LEU | A | 856 | 18.968 | −5.676 | −2.643 | 1.00 | 15.60 |
| ATOM | 2602 | CD1 | LEU | A | 856 | 18.996 | −4.549 | −1.634 | 1.00 | 19.73 |
| ATOM | 2603 | CD2 | LEU | A | 856 | 19.226 | −5.145 | −4.047 | 1.00 | 12.95 |
| ATOM | 2604 | C | LEU | A | 856 | 21.362 | −8.754 | −2.835 | 1.00 | 17.35 |
| ATOM | 2605 | O | LEU | A | 856 | 22.478 | −8.601 | −3.337 | 1.00 | 17.30 |
| ATOM | 2606 | N | ALA | A | 857 | 21.109 | −9.674 | −1.911 | 1.00 | 16.52 |
| ATOM | 2607 | CA | ALA | A | 857 | 22.156 | −10.545 | −1.396 | 1.00 | 17.82 |
| ATOM | 2608 | CB | ALA | A | 857 | 21.708 | −11.155 | −0.079 | 1.00 | 15.78 |
| ATOM | 2609 | C | ALA | A | 857 | 22.616 | −11.651 | −2.339 | 1.00 | 19.95 |
| ATOM | 2610 | O | ALA | A | 857 | 23.781 | −12.048 | −2.305 | 1.00 | 21.30 |
| ATOM | 2611 | N | GLU | A | 858 | 21.715 | −12.144 | −3.181 | 1.00 | 19.75 |
| ATOM | 2612 | CA | GLU | A | 858 | 22.059 | −13.233 | −4.091 | 1.00 | 23.14 |
| ATOM | 2613 | CB | GLU | A | 858 | 20.798 | −14.008 | −4.475 | 1.00 | 28.23 |
| ATOM | 2614 | CG | GLU | A | 858 | 19.735 | −13.155 | −5.139 | 1.00 | 35.46 |
| ATOM | 2615 | CD | GLU | A | 858 | 18.516 | −13.957 | −5.548 | 1.00 | 41.30 |
| ATOM | 2616 | OE1 | GLU | A | 858 | 17.928 | −14.635 | −4.679 | 1.00 | 46.06 |
| ATOM | 2617 | OE2 | GLU | A | 858 | 18.144 | −13.905 | −6.739 | 1.00 | 53.40 |
| ATOM | 2618 | C | GLU | A | 858 | 22.773 | −12.775 | −5.356 | 1.00 | 24.66 |
| ATOM | 2619 | O | GLU | A | 858 | 22.769 | −11.561 | −5.640 | 1.00 | 27.06 |
| ATOM | 2620 | OXT | GLU | A | 858 | 23.319 | −13.654 | −6.053 | 1.00 | 33.90 |
| TER | 2621 | | GLU | A | 858 | | | | | |
| ATOM | 2622 | O | HOH | W | 1 | 14.659 | 12.373 | 18.930 | 1.00 | 11.90 |
| ATOM | 2623 | O | HOH | W | 2 | 9.029 | 0.616 | 15.467 | 1.00 | 15.60 |
| ATOM | 2624 | O | HOH | W | 3 | 17.339 | −0.310 | 24.269 | 1.00 | 9.87 |
| ATOM | 2625 | O | HOH | W | 4 | 17.787 | 8.492 | 22.312 | 1.00 | 9.91 |
| ATOM | 2626 | O | HOH | W | 5 | 22.322 | 0.101 | 41.405 | 1.00 | 10.58 |
| ATOM | 2627 | O | HOH | W | 6 | 7.037 | −4.929 | 2.938 | 1.00 | 15.52 |
| ATOM | 2628 | O | HOH | W | 7 | 24.844 | 0.942 | 17.890 | 1.00 | 12.19 |
| ATOM | 2629 | O | HOH | W | 8 | 17.812 | −5.285 | 21.940 | 1.00 | 13.09 |
| ATOM | 2630 | O | HOH | W | 9 | 19.355 | −1.244 | 21.749 | 1.00 | 11.09 |
| ATOM | 2631 | O | HOH | W | 10 | 11.998 | −15.929 | 13.123 | 1.00 | 15.08 |
| ATOM | 2632 | O | HOH | W | 11 | 17.595 | 6.868 | 5.858 | 1.00 | 17.40 |
| ATOM | 2633 | O | HOH | W | 12 | 20.522 | −8.124 | 28.490 | 1.00 | 16.30 |
| ATOM | 2634 | O | HOH | W | 13 | 7.122 | 8.426 | 30.706 | 1.00 | 12.74 |
| ATOM | 2635 | O | HOH | W | 14 | 14.281 | −0.736 | 24.882 | 1.00 | 10.20 |
| ATOM | 2636 | O | HOH | W | 15 | 27.778 | −4.812 | 7.714 | 1.00 | 14.34 |
| ATOM | 2637 | O | HOH | W | 16 | 45.697 | −5.856 | 19.373 | 1.00 | 13.79 |
| ATOM | 2638 | O | HOH | W | 17 | 32.028 | 0.851 | 11.859 | 1.00 | 12.49 |
| ATOM | 2639 | O | HOH | W | 18 | 12.716 | −7.833 | 22.260 | 1.00 | 11.62 |
| ATOM | 2640 | O | HOH | W | 19 | 20.770 | −7.019 | 24.953 | 1.00 | 13.66 |
| ATOM | 2641 | O | HOH | W | 20 | 9.679 | −8.387 | 30.457 | 1.00 | 18.04 |
| ATOM | 2642 | O | HOH | W | 21 | 20.996 | −1.566 | 19.584 | 1.00 | 15.27 |
| ATOM | 2643 | O | HOH | W | 22 | 5.367 | −8.570 | 29.548 | 1.00 | 17.36 |
| ATOM | 2644 | O | HOH | W | 23 | 25.138 | −9.337 | 1.725 | 1.00 | 17.63 |
| ATOM | 2645 | O | HOH | W | 24 | −9.020 | −0.116 | 43.708 | 1.00 | 15.91 |
| ATOM | 2646 | O | HOH | W | 25 | 22.657 | −6.174 | 29.194 | 1.00 | 17.18 |
| ATOM | 2647 | O | HOH | W | 26 | −4.280 | 19.557 | 21.192 | 1.00 | 19.85 |
| ATOM | 2648 | O | HOH | W | 27 | 14.289 | 6.604 | 13.852 | 1.00 | 14.85 |
| ATOM | 2649 | O | HOH | W | 28 | 11.411 | −1.302 | 13.935 | 1.00 | 12.80 |
| ATOM | 2650 | O | HOH | W | 29 | 11.700 | 13.882 | 15.200 | 1.00 | 14.97 |
| ATOM | 2651 | O | HOH | W | 30 | 18.410 | 10.076 | 12.196 | 1.00 | 19.59 |
| ATOM | 2652 | O | HOH | W | 31 | 26.709 | 7.571 | 23.378 | 1.00 | 17.44 |
| ATOM | 2653 | O | HOH | W | 32 | 25.420 | 5.805 | 17.773 | 1.00 | 17.40 |
| ATOM | 2654 | O | HOH | W | 33 | 11.087 | 10.589 | 10.070 | 1.00 | 17.81 |
| ATOM | 2655 | O | HOH | W | 34 | 11.500 | −19.954 | 26.231 | 1.00 | 16.07 |
| ATOM | 2656 | O | HOH | W | 35 | −5.204 | −5.042 | 34.106 | 1.00 | 17.24 |
| ATOM | 2657 | O | HOH | W | 36 | 1.941 | 9.940 | 36.428 | 1.00 | 13.95 |
| ATOM | 2658 | O | HOH | W | 37 | 23.916 | 7.238 | 15.924 | 1.00 | 14.00 |
| ATOM | 2659 | O | HOH | W | 38 | 4.522 | 21.473 | 19.911 | 1.00 | 20.60 |
| ATOM | 2660 | O | HOH | W | 39 | 7.654 | 32.374 | 24.586 | 1.00 | 19.77 |
| ATOM | 2661 | O | HOH | W | 40 | −11.105 | 8.617 | 12.732 | 1.00 | 20.19 |
| ATOM | 2662 | O | HOH | W | 41 | 14.266 | 14.289 | 20.855 | 1.00 | 16.50 |
| ATOM | 2663 | O | HOH | W | 42 | 27.797 | −9.140 | 13.072 | 1.00 | 17.28 |
| ATOM | 2664 | O | HOH | W | 43 | 13.361 | −5.781 | 20.636 | 1.00 | 17.62 |
| ATOM | 2665 | O | HOH | W | 44 | 9.439 | 17.952 | 22.920 | 1.00 | 20.34 |
| ATOM | 2666 | O | HOH | W | 45 | 13.506 | −19.339 | 23.851 | 1.00 | 17.51 |
| ATOM | 2667 | O | HOH | W | 46 | −9.608 | −2.484 | 44.794 | 1.00 | 18.03 |
| ATOM | 2668 | O | HOH | W | 47 | 19.179 | −3.517 | 23.479 | 1.00 | 14.63 |
| ATOM | 2669 | O | HOH | W | 48 | 20.825 | −1.771 | 7.357 | 1.00 | 15.56 |
| ATOM | 2670 | O | HOH | W | 49 | 3.953 | −2.401 | 8.103 | 1.00 | 15.98 |
| ATOM | 2671 | O | HOH | W | 50 | 5.164 | 2.828 | 12.389 | 1.00 | 17.57 |
| ATOM | 2672 | O | HOH | W | 51 | 22.475 | −6.004 | 8.390 | 1.00 | 20.31 |
| ATOM | 2673 | O | HOH | W | 52 | −1.505 | −7.713 | 3.922 | 1.00 | 20.93 |
| ATOM | 2674 | O | HOH | W | 53 | 16.030 | 13.344 | 15.066 | 1.00 | 22.86 |
| ATOM | 2675 | O | HOH | W | 54 | 3.120 | 33.114 | 17.327 | 1.00 | 18.81 |
| ATOM | 2676 | O | HOH | W | 55 | 6.517 | 19.676 | 19.112 | 1.00 | 20.43 |
| ATOM | 2677 | O | HOH | W | 56 | 27.812 | 5.941 | 11.269 | 1.00 | 19.39 |
| ATOM | 2678 | O | HOH | W | 57 | 14.704 | 9.138 | 12.638 | 1.00 | 17.37 |
| ATOM | 2679 | O | HOH | W | 58 | 10.813 | −19.129 | 28.734 | 1.00 | 23.50 |
| ATOM | 2680 | O | HOH | W | 59 | 9.603 | −13.958 | 1.403 | 1.00 | 20.05 |
| ATOM | 2681 | O | HOH | W | 60 | 46.209 | −3.550 | 17.835 | 1.00 | 18.22 |
| ATOM | 2682 | O | HOH | W | 61 | 24.760 | −2.396 | 10.006 | 1.00 | 20.80 |
| ATOM | 2683 | O | HOH | W | 62 | 11.077 | 3.087 | 12.585 | 1.00 | 21.05 |
| ATOM | 2684 | O | HOH | W | 63 | 10.534 | −10.726 | 28.998 | 1.00 | 21.18 |
| ATOM | 2685 | O | HOH | W | 64 | −0.961 | −11.064 | 33.440 | 1.00 | 18.63 |
| ATOM | 2686 | O | HOH | W | 65 | 20.890 | 0.577 | 15.388 | 1.00 | 19.77 |
| ATOM | 2687 | O | HOH | W | 66 | 21.685 | 13.866 | 20.281 | 1.00 | 24.54 |
| ATOM | 2688 | O | HOH | W | 67 | −5.012 | −3.878 | 8.180 | 1.00 | 21.24 |
| ATOM | 2689 | O | HOH | W | 68 | −10.443 | 9.371 | 15.194 | 1.00 | 19.03 |
| ATOM | 2690 | O | HOH | W | 69 | 26.635 | 8.578 | 39.152 | 1.00 | 19.48 |
| ATOM | 2691 | O | HOH | W | 70 | 21.837 | −4.044 | 23.094 | 1.00 | 14.46 |
| ATOM | 2692 | O | HOH | W | 71 | 18.979 | 0.534 | 3.275 | 1.00 | 19.32 |
| ATOM | 2693 | O | HOH | W | 72 | −7.687 | −4.529 | 35.484 | 1.00 | 19.76 |
| ATOM | 2694 | O | HOH | W | 73 | −4.441 | 9.972 | 35.483 | 1.00 | 21.75 |
| ATOM | 2695 | O | HOH | W | 74 | 22.147 | 0.611 | 18.233 | 1.00 | 21.06 |
| ATOM | 2696 | O | HOH | W | 75 | 25.625 | 7.694 | 19.834 | 1.00 | 17.79 |
| ATOM | 2697 | O | HOH | W | 76 | −9.716 | 14.941 | 11.027 | 1.00 | 20.22 |
| ATOM | 2698 | O | HOH | W | 77 | 14.015 | −1.710 | 16.866 | 1.00 | 19.03 |
| ATOM | 2699 | O | HOH | W | 78 | 21.861 | −3.953 | 20.354 | 1.00 | 17.68 |
| ATOM | 2700 | O | HOH | W | 79 | 21.854 | −11.471 | 10.659 | 1.00 | 21.05 |
| ATOM | 2701 | O | HOH | W | 80 | 15.795 | −5.944 | −4.295 | 1.00 | 17.34 |
| ATOM | 2702 | O | HOH | W | 81 | 26.427 | −5.333 | 20.748 | 1.00 | 28.65 |
| ATOM | 2703 | O | HOH | W | 82 | 0.806 | 16.637 | 36.547 | 1.00 | 22.11 |
| ATOM | 2704 | O | HOH | W | 83 | 28.883 | 5.909 | 33.300 | 1.00 | 35.99 |
| ATOM | 2705 | O | HOH | W | 84 | 0.615 | −5.836 | 36.804 | 1.00 | 21.63 |
| ATOM | 2706 | O | HOH | W | 85 | 14.416 | −12.696 | −3.692 | 1.00 | 22.36 |
| ATOM | 2707 | O | HOH | W | 86 | −4.841 | 7.644 | 43.980 | 1.00 | 19.92 |
| ATOM | 2708 | O | HOH | W | 87 | −6.059 | 5.564 | 10.293 | 1.00 | 22.49 |
| ATOM | 2709 | O | HOH | W | 88 | 7.210 | 1.031 | 13.002 | 1.00 | 19.78 |
| ATOM | 2710 | O | HOH | W | 89 | −5.786 | −6.329 | 9.023 | 1.00 | 20.32 |
| ATOM | 2711 | O | HOH | W | 90 | 19.117 | 4.909 | 35.027 | 1.00 | 17.30 |
| ATOM | 2712 | O | HOH | W | 91 | 10.592 | 13.017 | 38.293 | 1.00 | 23.66 |
| ATOM | 2713 | O | HOH | W | 92 | −9.801 | 5.234 | 16.132 | 1.00 | 20.64 |
| ATOM | 2714 | O | HOH | W | 93 | −6.370 | −17.172 | 24.540 | 1.00 | 23.65 |
| ATOM | 2715 | O | HOH | W | 94 | −6.833 | 1.342 | 45.207 | 1.00 | 17.92 |
| ATOM | 2716 | O | HOH | W | 95 | 33.715 | −7.279 | 11.000 | 1.00 | 17.67 |
| ATOM | 2717 | O | HOH | W | 96 | 5.722 | 31.659 | 29.252 | 1.00 | 19.35 |
| ATOM | 2718 | O | HOH | W | 97 | 14.155 | −2.890 | 10.083 | 1.00 | 16.30 |
| ATOM | 2719 | O | HOH | W | 98 | 19.709 | −5.898 | 20.414 | 1.00 | 27.45 |
| ATOM | 2720 | O | HOH | W | 99 | 4.418 | −3.958 | 2.629 | 1.00 | 23.70 |
| ATOM | 2721 | O | HOH | W | 100 | 5.739 | 5.419 | 41.566 | 1.00 | 26.30 |
| ATOM | 2722 | O | HOH | W | 101 | 1.882 | −10.334 | 12.547 | 1.00 | 21.56 |
| ATOM | 2723 | O | HOH | W | 102 | 1.506 | 9.558 | 7.516 | 1.00 | 21.76 |
| ATOM | 2724 | O | HOH | W | 103 | 23.211 | 1.757 | 4.698 | 1.00 | 23.21 |
| ATOM | 2725 | O | HOH | W | 104 | 36.282 | 2.363 | 9.440 | 1.00 | 27.13 |
| ATOM | 2726 | O | HOH | W | 105 | 27.600 | −11.267 | 2.472 | 1.00 | 18.59 |
| ATOM | 2727 | O | HOH | W | 106 | 16.305 | 16.757 | 22.862 | 1.00 | 25.14 |
| ATOM | 2728 | O | HOH | W | 107 | 21.223 | −0.443 | 4.807 | 1.00 | 20.51 |
| ATOM | 2729 | O | HOH | W | 108 | 12.939 | 13.840 | 12.680 | 1.00 | 26.10 |
| ATOM | 2730 | O | HOH | W | 109 | 10.191 | −9.652 | 24.549 | 1.00 | 17.97 |
| ATOM | 2731 | O | HOH | W | 110 | 9.527 | −19.990 | 11.160 | 1.00 | 34.52 |
| ATOM | 2732 | O | HOH | W | 111 | 2.300 | −4.117 | 6.982 | 1.00 | 21.40 |
| ATOM | 2733 | O | HOH | W | 112 | 31.807 | 5.599 | 25.384 | 1.00 | 19.28 |
| ATOM | 2734 | O | HOH | W | 113 | 11.858 | 5.625 | 12.858 | 1.00 | 18.80 |
| ATOM | 2735 | O | HOH | W | 114 | 2.975 | 3.851 | 3.583 | 1.00 | 28.02 |
| ATOM | 2736 | O | HOH | W | 115 | 22.328 | −3.325 | 9.278 | 1.00 | 18.13 |
| ATOM | 2737 | O | HOH | W | 116 | 7.581 | 21.278 | 17.210 | 1.00 | 26.33 |
| ATOM | 2738 | O | HOH | W | 117 | −8.959 | 18.330 | 19.702 | 1.00 | 25.41 |
| ATOM | 2739 | O | HOH | W | 118 | −7.913 | −3.625 | 46.688 | 1.00 | 16.16 |
| ATOM | 2740 | O | HOH | W | 119 | 29.369 | −4.122 | 35.016 | 1.00 | 21.35 |
| ATOM | 2741 | O | HOH | W | 120 | −10.313 | 4.301 | 39.676 | 1.00 | 20.84 |
| ATOM | 2742 | O | HOH | W | 121 | 36.288 | −8.163 | 10.383 | 1.00 | 29.41 |
| ATOM | 2743 | O | HOH | W | 122 | −5.305 | 0.154 | 7.066 | 1.00 | 32.03 |
| ATOM | 2744 | O | HOH | W | 123 | 15.892 | 9.345 | 6.948 | 1.00 | 31.98 |
| ATOM | 2745 | O | HOH | W | 124 | −7.934 | −11.981 | 26.594 | 1.00 | 24.88 |
| ATOM | 2746 | O | HOH | W | 125 | −7.700 | −14.762 | 14.417 | 1.00 | 27.16 |
| ATOM | 2747 | O | HOH | W | 126 | 7.924 | 19.345 | 32.073 | 1.00 | 27.10 |
| ATOM | 2748 | O | HOH | W | 127 | 22.963 | −15.271 | −0.194 | 1.00 | 34.57 |
| ATOM | 2749 | O | HOH | W | 128 | 14.018 | 4.418 | 0.344 | 1.00 | 27.94 |
| ATOM | 2750 | O | HOH | W | 129 | 13.334 | 16.377 | 17.301 | 1.00 | 27.05 |
| ATOM | 2751 | O | HOH | W | 130 | 30.641 | −11.391 | 0.176 | 1.00 | 21.79 |
| ATOM | 2752 | O | HOH | W | 131 | −5.143 | −9.116 | 6.397 | 1.00 | 26.22 |
| ATOM | 2753 | O | HOH | W | 132 | 44.452 | 2.118 | 22.189 | 1.00 | 25.35 |
| ATOM | 2754 | O | HOH | W | 133 | −0.942 | −8.268 | 32.526 | 1.00 | 21.73 |
| ATOM | 2755 | O | HOH | W | 134 | 32.286 | 8.100 | 17.531 | 1.00 | 30.18 |
| ATOM | 2756 | O | HOH | W | 135 | 4.471 | −22.268 | 17.254 | 1.00 | 22.18 |
| ATOM | 2757 | O | HOH | W | 136 | 20.964 | −7.154 | 10.051 | 1.00 | 23.34 |
| ATOM | 2758 | O | HOH | W | 137 | −6.984 | 26.107 | 22.746 | 1.00 | 26.33 |
| ATOM | 2759 | O | HOH | W | 138 | 17.856 | 14.348 | 24.807 | 1.00 | 20.33 |
| ATOM | 2760 | O | HOH | W | 139 | 10.038 | −5.546 | 41.193 | 1.00 | 26.62 |
| ATOM | 2761 | O | HOH | W | 140 | 13.989 | 13.837 | 16.687 | 1.00 | 27.53 |
| ATOM | 2762 | O | HOH | W | 141 | 23.629 | −13.084 | 9.502 | 1.00 | 28.21 |
| ATOM | 2763 | O | HOH | W | 142 | 31.635 | 7.713 | 14.307 | 1.00 | 30.30 |
| ATOM | 2764 | O | HOH | W | 143 | 6.811 | −2.344 | −0.868 | 1.00 | 24.47 |
| ATOM | 2765 | O | HOH | W | 144 | 7.278 | −9.981 | 30.756 | 1.00 | 27.03 |
| ATOM | 2766 | O | HOH | W | 145 | −6.981 | 7.089 | 6.281 | 1.00 | 26.94 |
| ATOM | 2767 | O | HOH | W | 146 | −8.635 | 18.558 | 29.861 | 1.00 | 27.91 |
| ATOM | 2768 | O | HOH | W | 147 | 30.525 | −1.680 | 34.334 | 1.00 | 24.09 |
| ATOM | 2769 | O | HOH | W | 148 | 2.251 | −13.867 | 2.121 | 1.00 | 25.49 |
| ATOM | 2770 | O | HOH | W | 149 | −8.063 | −9.740 | 29.191 | 1.00 | 24.34 |
| ATOM | 2771 | O | HOH | W | 150 | −1.333 | 15.279 | 37.020 | 1.00 | 28.81 |
| ATOM | 2772 | O | HOH | W | 151 | 18.429 | 15.610 | 21.408 | 1.00 | 22.50 |
| ATOM | 2773 | O | HOH | W | 152 | 13.471 | −4.264 | 12.528 | 1.00 | 19.38 |
| ATOM | 2774 | O | HOH | W | 153 | 18.621 | −17.600 | 7.222 | 1.00 | 21.52 |
| ATOM | 2775 | O | HOH | W | 154 | 19.181 | −5.396 | 13.023 | 1.00 | 33.83 |
| ATOM | 2776 | O | HOH | W | 155 | −3.698 | −11.419 | 33.622 | 1.00 | 22.32 |
| ATOM | 2777 | O | HOH | W | 156 | 24.773 | −7.687 | 26.828 | 1.00 | 25.23 |
| ATOM | 2778 | O | HOH | W | 157 | 11.338 | 6.981 | 10.450 | 1.00 | 25.98 |
| ATOM | 2779 | O | HOH | W | 158 | 7.187 | 4.097 | 9.722 | 1.00 | 25.73 |
| ATOM | 2780 | O | HOH | W | 159 | 10.571 | 18.276 | 18.940 | 1.00 | 26.56 |
| ATOM | 2781 | O | HOH | W | 160 | 29.444 | 8.138 | 18.655 | 1.00 | 25.10 |
| ATOM | 2782 | O | HOH | W | 161 | −4.733 | −16.782 | 12.321 | 1.00 | 23.51 |
| ATOM | 2783 | O | HOH | W | 162 | 24.194 | 7.218 | 40.602 | 1.00 | 28.88 |
| ATOM | 2784 | O | HOH | W | 163 | 13.925 | −7.040 | −5.961 | 1.00 | 29.58 |
| ATOM | 2785 | O | HOH | W | 164 | −8.456 | 17.425 | 10.469 | 1.00 | 25.03 |
| ATOM | 2786 | O | HOH | W | 165 | 20.283 | 7.809 | 6.109 | 1.00 | 25.58 |
| ATOM | 2787 | O | HOH | W | 166 | 11.226 | 1.360 | 14.389 | 1.00 | 28.28 |
| ATOM | 2788 | O | HOH | W | 167 | 17.600 | 11.985 | 25.766 | 1.00 | 28.21 |
| ATOM | 2789 | O | HOH | W | 168 | −1.369 | 0.364 | 6.150 | 1.00 | 26.61 |
| ATOM | 2790 | O | HOH | W | 169 | 1.250 | 6.912 | 6.112 | 1.00 | 22.96 |
| ATOM | 2791 | O | HOH | W | 170 | 13.982 | 16.820 | 19.827 | 1.00 | 26.70 |
| ATOM | 2792 | O | HOH | W | 171 | 8.401 | 6.117 | 8.285 | 1.00 | 29.37 |
| ATOM | 2793 | O | HOH | W | 172 | 1.561 | 32.980 | 20.315 | 1.00 | 25.71 |
| ATOM | 2794 | O | HOH | W | 173 | 8.687 | −4.960 | 0.724 | 1.00 | 20.63 |
| ATOM | 2795 | O | HOH | W | 174 | 16.762 | −8.584 | 32.541 | 1.00 | 22.53 |
| ATOM | 2796 | O | HOH | W | 175 | −8.928 | 23.790 | 25.122 | 1.00 | 26.68 |
| ATOM | 2797 | O | HOH | W | 176 | 30.774 | −10.692 | 12.781 | 1.00 | 29.33 |
| ATOM | 2798 | O | HOH | W | 177 | −4.491 | −0.583 | 48.435 | 1.00 | 27.59 |
| ATOM | 2799 | O | HOH | W | 178 | −10.254 | 8.887 | 25.677 | 1.00 | 27.41 |
| ATOM | 2800 | O | HOH | W | 179 | 12.487 | −14.347 | 0.366 | 1.00 | 29.02 |
| ATOM | 2801 | O | HOH | W | 180 | −2.194 | −3.193 | 5.572 | 1.00 | 37.26 |
| ATOM | 2802 | O | HOH | W | 181 | 21.240 | −14.747 | 4.723 | 1.00 | 24.12 |
| ATOM | 2803 | O | HOH | W | 182 | 25.238 | 15.872 | 21.083 | 1.00 | 33.15 |
| ATOM | 2804 | O | HOH | W | 183 | 27.650 | 8.541 | 12.497 | 1.00 | 23.15 |
| ATOM | 2805 | O | HOH | W | 184 | 35.303 | 3.837 | 26.688 | 1.00 | 23.28 |
| ATOM | 2806 | O | HOH | W | 185 | −3.730 | 14.555 | 34.107 | 1.00 | 23.34 |
| ATOM | 2807 | O | HOH | W | 186 | −8.927 | −4.660 | 10.381 | 1.00 | 28.70 |
| ATOM | 2808 | O | HOH | W | 187 | 40.695 | −1.179 | 15.166 | 1.00 | 31.35 |
| ATOM | 2809 | O | HOH | W | 188 | −0.356 | −22.615 | 31.607 | 1.00 | 35.28 |
| ATOM | 2810 | O | HOH | W | 189 | 3.463 | 34.674 | 22.382 | 1.00 | 27.20 |
| ATOM | 2811 | O | HOH | W | 190 | 8.911 | 2.196 | 11.376 | 1.00 | 19.30 |
| ATOM | 2812 | O | HOH | W | 191 | −2.598 | −24.115 | 28.861 | 1.00 | 26.04 |
| ATOM | 2813 | O | HOH | W | 192 | 8.418 | −15.510 | 34.179 | 1.00 | 38.02 |
| ATOM | 2814 | O | HOH | W | 193 | 15.703 | −17.633 | −0.060 | 1.00 | 35.88 |
| ATOM | 2815 | O | HOH | W | 194 | 32.518 | 0.997 | 3.614 | 1.00 | 34.25 |
| ATOM | 2816 | O | HOH | W | 195 | 19.062 | −16.798 | 10.198 | 1.00 | 27.69 |
| ATOM | 2817 | O | HOH | W | 196 | 13.216 | 8.976 | 10.392 | 1.00 | 27.61 |
| ATOM | 2818 | O | HOH | W | 197 | 2.647 | −0.086 | 12.019 | 1.00 | 23.91 |
| ATOM | 2819 | O | HOH | W | 198 | −1.276 | −7.561 | 35.325 | 1.00 | 25.96 |
| ATOM | 2820 | O | HOH | W | 199 | 4.267 | −22.362 | 30.170 | 1.00 | 35.68 |
| ATOM | 2821 | O | HOH | W | 200 | 21.787 | 10.595 | 30.558 | 1.00 | 32.19 |
| ATOM | 2822 | O | HOH | W | 201 | 21.638 | 20.389 | 14.210 | 1.00 | 43.09 |
| ATOM | 2823 | O | HOH | W | 202 | 5.373 | −10.297 | 38.927 | 1.00 | 36.29 |
| ATOM | 2824 | O | HOH | W | 203 | 29.035 | −9.588 | 1.139 | 1.00 | 25.61 |
| ATOM | 2825 | O | HOH | W | 204 | 0.403 | −17.629 | 11.462 | 1.00 | 25.52 |
| ATOM | 2826 | O | HOH | W | 205 | 15.896 | −2.824 | 23.429 | 1.00 | 9.81 |
| ATOM | 2827 | O | HOH | W | 206 | 13.783 | −1.283 | 21.923 | 1.00 | 12.03 |
| ATOM | 2828 | O | HOH | W | 207 | 16.854 | −0.738 | 21.328 | 1.00 | 11.76 |
| ATOM | 2829 | O | HOH | W | 208 | 14.946 | −0.465 | 19.263 | 1.00 | 10.55 |
| ATOM | 2830 | O | HOH | W | 209 | −5.566 | −9.500 | 29.835 | 1.00 | 34.12 |
| ATOM | 2831 | O | HOH | W | 210 | 11.435 | 14.251 | 33.693 | 1.00 | 48.96 |
| ATOM | 2832 | O | HOH | W | 211 | 3.944 | 31.281 | 18.816 | 1.00 | 43.83 |
| ATOM | 2833 | O | HOH | W | 212 | 21.511 | 8.201 | 40.401 | 1.00 | 42.14 |
| ATOM | 2834 | O | HOH | W | 213 | 44.825 | 1.178 | 19.793 | 1.00 | 52.54 |
| ATOM | 2835 | O | HOH | W | 214 | 9.397 | 20.244 | 23.958 | 1.00 | 30.91 |
| ATOM | 2836 | O | HOH | W | 215 | 18.636 | −1.182 | 15.865 | 1.00 | 23.82 |
| ATOM | 2837 | O | HOH | W | 216 | 18.955 | −2.693 | 17.957 | 1.00 | 28.82 |
| ATOM | 2838 | O | HOH | W | 217 | 26.248 | −9.541 | 27.965 | 1.00 | 25.10 |
| ATOM | 2839 | O | HOH | W | 218 | 24.703 | −9.867 | 30.146 | 1.00 | 25.52 |
| ATOM | 2840 | O | HOH | W | 219 | 22.600 | −9.260 | 25.832 | 1.00 | 26.71 |
| ATOM | 2841 | O | HOH | W | 220 | 1.825 | 10.054 | 39.457 | 1.00 | 25.12 |
| ATOM | 2842 | O | HOH | W | 221 | 3.585 | 4.590 | 45.058 | 1.00 | 28.59 |
| ATOM | 2843 | O | HOH | W | 222 | 3.777 | −6.424 | 30.447 | 1.00 | 24.42 |
| TER | 2844 | | HOH | W | 222 | | | | | |
| ATOM | 2845 | ZN | ZN | B | 864 | 13.210 | 0.069 | 20.406 | 1.00 | 15.15 |
| ATOM | 2846 | MG | MG | B | 865 | 15.487 | −0.725 | 23.006 | 1.00 | 8.84 |
| TER | 2847 | | MG | B | 865 | | | | | |
| END | | | | | | | | | | |

TABLE 5
Atomic coordinates for PDE5* complex with UK-088,800
Atom Number, Atom Type, Residue Type, Residue Number Cartesian Coordinates
X, Y, Z, Atom Occupancy (O), Temperature Factor (B) and Atom Type

| | | | | | | X | Y | Z | O | B |
|---|---|---|---|---|---|---|---|---|---|---|
| ATOM | 1 | CB | GLU | A | 536 | 9.887 | 25.182 | 24.787 | 1.00 | 33.98 |
| ATOM | 2 | CG | GLU | A | 536 | 11.305 | 25.697 | 24.635 | 1.00 | 42.32 |
| ATOM | 3 | CD | GLU | A | 536 | 11.492 | 27.074 | 25.245 | 1.00 | 47.12 |
| ATOM | 4 | OE1 | GLU | A | 536 | 11.290 | 27.223 | 26.470 | 1.00 | 54.45 |
| ATOM | 5 | OE2 | GLU | A | 536 | 11.842 | 28.012 | 24.499 | 1.00 | 51.15 |
| ATOM | 6 | C | GLU | A | 536 | 7.942 | 25.044 | 26.327 | 1.00 | 37.22 |
| ATOM | 7 | O | GLU | A | 536 | 7.222 | 24.272 | 25.690 | 1.00 | 36.76 |
| ATOM | 8 | N | GLU | A | 536 | 9.991 | 23.667 | 26.752 | 1.00 | 36.93 |
| ATOM | 9 | CA | GLU | A | 536 | 9.456 | 24.961 | 26.239 | 1.00 | 37.47 |
| ATOM | 10 | N | THR | A | 537 | 7.463 | 25.990 | 27.125 | 1.00 | 37.34 |
| ATOM | 11 | CA | THR | A | 537 | 6.035 | 26.189 | 27.289 | 1.00 | 36.78 |
| ATOM | 12 | CB | THR | A | 537 | 5.750 | 27.235 | 28.387 | 1.00 | 43.61 |
| ATOM | 13 | OG1 | THR | A | 537 | 6.425 | 28.457 | 28.069 | 1.00 | 45.23 |
| ATOM | 14 | CG2 | THR | A | 537 | 6.241 | 26.731 | 29.739 | 1.00 | 34.90 |
| ATOM | 15 | C | THR | A | 537 | 5.452 | 26.660 | 25.957 | 1.00 | 35.15 |
| ATOM | 16 | O | THR | A | 537 | 4.239 | 26.661 | 25.764 | 1.00 | 34.30 |
| ATOM | 17 | N | ARG | A | 538 | 6.336 | 27.046 | 25.040 | 1.00 | 32.72 |
| ATOM | 18 | CA | ARG | A | 538 | 5.943 | 27.514 | 23.712 | 1.00 | 31.35 |
| ATOM | 19 | CB | ARG | A | 538 | 7.143 | 28.133 | 23.002 | 1.00 | 33.78 |
| ATOM | 20 | CG | ARG | A | 538 | 6.910 | 28.452 | 21.531 | 1.00 | 42.77 |
| ATOM | 21 | CD | ARG | A | 538 | 6.041 | 29.690 | 21.340 | 1.00 | 51.85 |
| ATOM | 22 | NE | ARG | A | 538 | 4.638 | 29.475 | 21.687 | 1.00 | 50.21 |
| ATOM | 23 | CZ | ARG | A | 538 | 3.715 | 30.433 | 21.672 | 1.00 | 49.57 |
| ATOM | 24 | NH1 | ARG | A | 538 | 4.047 | 31.673 | 21.330 | 1.00 | 48.08 |
| ATOM | 25 | NH2 | ARG | A | 538 | 2.458 | 30.154 | 21.992 | 1.00 | 49.14 |
| ATOM | 26 | C | ARG | A | 538 | 5.405 | 26.369 | 22.861 | 1.00 | 30.33 |
| ATOM | 27 | O | ARG | A | 538 | 4.381 | 26.501 | 22.187 | 1.00 | 28.72 |
| ATOM | 28 | N | GLU | A | 539 | 6.120 | 25.250 | 22.876 | 1.00 | 28.94 |
| ATOM | 29 | CA | GLU | A | 539 | 5.709 | 24.080 | 22.115 | 1.00 | 25.47 |
| ATOM | 30 | CB | GLU | A | 539 | 6.807 | 23.024 | 22.141 | 1.00 | 30.35 |
| ATOM | 31 | CG | GLU | A | 539 | 8.055 | 23.426 | 21.391 | 1.00 | 31.75 |
| ATOM | 32 | CD | GLU | A | 539 | 9.188 | 22.463 | 21.628 | 1.00 | 31.48 |
| ATOM | 33 | OE1 | GLU | A | 539 | 9.689 | 22.410 | 22.771 | 1.00 | 46.55 |
| ATOM | 34 | OE2 | GLU | A | 539 | 9.575 | 21.754 | 20.676 | 1.00 | 57.06 |
| ATOM | 35 | C | GLU | A | 539 | 4.437 | 23.511 | 22.718 | 1.00 | 23.49 |
| ATOM | 36 | O | GLU | A | 539 | 3.599 | 22.952 | 22.011 | 1.00 | 21.01 |
| ATOM | 37 | N | LEU | A | 540 | 4.299 | 23.653 | 24.032 | 1.00 | 22.37 |
| ATOM | 38 | CA | LEU | A | 540 | 3.114 | 23.159 | 24.713 | 1.00 | 22.10 |
| ATOM | 39 | CB | LEU | A | 540 | 3.302 | 23.223 | 26.231 | 1.00 | 19.44 |
| ATOM | 40 | CG | LEU | A | 540 | 2.098 | 22.763 | 27.059 | 1.00 | 22.04 |
| ATOM | 41 | CD1 | LEU | A | 540 | 1.686 | 21.365 | 26.627 | 1.00 | 19.81 |
| ATOM | 42 | CD2 | LEU | A | 540 | 2.441 | 22.783 | 28.539 | 1.00 | 38.12 |
| ATOM | 43 | C | LEU | A | 540 | 1.907 | 23.995 | 24.300 | 1.00 | 21.23 |
| ATOM | 44 | O | LEU | A | 540 | 0.844 | 23.461 | 23.990 | 1.00 | 19.37 |
| ATOM | 45 | N | GLN | A | 541 | 2.074 | 25.313 | 24.283 | 1.00 | 20.36 |
| ATOM | 46 | CA | GLN | A | 541 | 0.972 | 26.185 | 23.905 | 1.00 | 18.42 |
| ATOM | 47 | CB | GLN | A | 541 | 1.410 | 27.651 | 23.966 | 1.00 | 20.67 |
| ATOM | 48 | CG | GLN | A | 541 | 1.873 | 28.081 | 25.350 | 1.00 | 24.12 |
| ATOM | 49 | CD | GLN | A | 541 | 2.356 | 29.519 | 25.401 | 1.00 | 24.37 |
| ATOM | 50 | OE1 | GLN | A | 541 | 3.047 | 29.986 | 24.497 | 1.00 | 17.86 |
| ATOM | 51 | NE2 | GLN | A | 541 | 2.008 | 30.223 | 26.474 | 1.00 | 20.12 |
| ATOM | 52 | C | GLN | A | 541 | 0.477 | 25.833 | 22.505 | 1.00 | 17.74 |
| ATOM | 53 | O | GLN | A | 541 | −0.726 | 25.799 | 22.254 | 1.00 | 19.08 |
| ATOM | 54 | N | SER | A | 542 | 1.404 | 25.552 | 21.596 | 1.00 | 15.82 |
| ATOM | 55 | CA | SER | A | 542 | 1.026 | 25.207 | 20.231 | 1.00 | 18.42 |
| ATOM | 56 | CB | SER | A | 542 | 2.263 | 25.179 | 19.329 | 1.00 | 20.91 |
| ATOM | 57 | OG | SER | A | 542 | 2.857 | 26.463 | 19.251 | 1.00 | 37.22 |
| ATOM | 58 | C | SER | A | 542 | 0.320 | 23.858 | 20.174 | 1.00 | 16.82 |
| ATOM | 59 | O | SER | A | 542 | −0.710 | 23.709 | 19.520 | 1.00 | 17.71 |
| ATOM | 60 | N | LEU | A | 543 | 0.875 | 22.874 | 20.872 | 1.00 | 17.64 |
| ATOM | 61 | CA | LEU | A | 543 | 0.297 | 21.538 | 20.878 | 1.00 | 17.88 |
| ATOM | 62 | CB | LEU | A | 543 | 1.269 | 20.550 | 21.531 | 1.00 | 13.54 |
| ATOM | 63 | CG | LEU | A | 543 | 0.762 | 19.113 | 21.695 | 1.00 | 17.02 |
| ATOM | 64 | CD1 | LEU | A | 543 | 0.501 | 18.504 | 20.329 | 1.00 | 14.15 |
| ATOM | 65 | CD2 | LEU | A | 543 | 1.789 | 18.284 | 22.465 | 1.00 | 10.59 |
| ATOM | 66 | C | LEU | A | 543 | −1.049 | 21.468 | 21.592 | 1.00 | 18.33 |
| ATOM | 67 | O | LEU | A | 543 | −2.013 | 20.914 | 21.064 | 1.00 | 19.18 |
| ATOM | 68 | N | ALA | A | 544 | −1.112 | 22.038 | 22.790 | 1.00 | 18.60 |
| ATOM | 69 | CA | ALA | A | 544 | −2.332 | 22.003 | 23.588 | 1.00 | 19.09 |
| ATOM | 70 | CB | ALA | A | 544 | −2.053 | 22.573 | 24.972 | 1.00 | 17.83 |
| ATOM | 71 | C | ALA | A | 544 | −3.527 | 22.717 | 22.967 | 1.00 | 21.56 |
| ATOM | 72 | O | ALA | A | 544 | −4.667 | 22.306 | 23.165 | 1.00 | 23.51 |
| ATOM | 73 | N | ALA | A | 545 | −3.269 | 23.782 | 22.217 | 1.00 | 23.98 |
| ATOM | 74 | CA | ALA | A | 545 | −4.348 | 24.547 | 21.599 | 1.00 | 24.34 |
| ATOM | 75 | CB | ALA | A | 545 | −3.861 | 25.952 | 21.274 | 1.00 | 26.53 |
| ATOM | 76 | C | ALA | A | 545 | −4.901 | 23.891 | 20.341 | 1.00 | 25.76 |
| ATOM | 77 | O | ALA | A | 545 | −6.065 | 24.079 | 19.992 | 1.00 | 28.16 |
| ATOM | 78 | N | ALA | A | 546 | −4.059 | 23.115 | 19.668 | 1.00 | 24.74 |
| ATOM | 79 | CA | ALA | A | 546 | −4.441 | 22.443 | 18.430 | 1.00 | 24.22 |
| ATOM | 80 | CB | ALA | A | 546 | −3.239 | 21.701 | 17.861 | 1.00 | 22.00 |
| ATOM | 81 | C | ALA | A | 546 | −5.621 | 21.485 | 18.529 | 1.00 | 21.89 |
| ATOM | 82 | O | ALA | A | 546 | −5.803 | 20.791 | 19.529 | 1.00 | 23.27 |
| ATOM | 83 | N | VAL | A | 547 | −6.433 | 21.464 | 17.478 | 1.00 | 21.72 |
| ATOM | 84 | CA | VAL | A | 547 | −7.560 | 20.548 | 17.418 | 1.00 | 21.65 |
| ATOM | 85 | CB | VAL | A | 547 | −8.660 | 21.045 | 16.454 | 1.00 | 23.33 |
| ATOM | 86 | CG1 | VAL | A | 547 | −9.754 | 19.993 | 16.326 | 1.00 | 25.11 |
| ATOM | 87 | CG2 | VAL | A | 547 | −9.250 | 22.355 | 16.965 | 1.00 | 23.58 |
| ATOM | 88 | C | VAL | A | 547 | −6.925 | 19.284 | 16.850 | 1.00 | 22.28 |
| ATOM | 89 | O | VAL | A | 547 | −6.280 | 19.329 | 15.805 | 1.00 | 24.96 |
| ATOM | 90 | N | VAL | A | 548 | −7.090 | 18.165 | 17.542 | 1.00 | 21.82 |
| ATOM | 91 | CA | VAL | A | 548 | −6.498 | 16.909 | 17.098 | 1.00 | 19.25 |
| ATOM | 92 | CB | VAL | A | 548 | −6.387 | 15.925 | 18.278 | 1.00 | 19.56 |
| ATOM | 93 | CG1 | VAL | A | 548 | −5.669 | 14.659 | 17.840 | 1.00 | 23.78 |
| ATOM | 94 | CG2 | VAL | A | 548 | −5.656 | 16.593 | 19.438 | 1.00 | 24.95 |
| ATOM | 95 | C | VAL | A | 548 | −7.284 | 16.241 | 15.975 | 1.00 | 17.26 |
| ATOM | 96 | O | VAL | A | 548 | −8.453 | 15.904 | 16.146 | 1.00 | 19.24 |
| ATOM | 97 | N | PRO | A | 549 | −6.646 | 16.038 | 14.808 | 1.00 | 17.33 |
| ATOM | 98 | CD | PRO | A | 549 | −5.293 | 16.478 | 14.431 | 1.00 | 18.64 |
| ATOM | 99 | CA | PRO | A | 549 | −7.315 | 15.401 | 13.670 | 1.00 | 19.43 |
| ATOM | 100 | CB | PRO | A | 549 | −6.245 | 15.425 | 12.575 | 1.00 | 17.30 |
| ATOM | 101 | CG | PRO | A | 549 | −5.420 | 16.615 | 12.931 | 1.00 | 23.24 |
| ATOM | 102 | C | PRO | A | 549 | −7.742 | 13.982 | 14.023 | 1.00 | 20.23 |
| ATOM | 103 | O | PRO | A | 549 | −7.259 | 13.402 | 15.001 | 1.00 | 16.82 |
| ATOM | 104 | N | SER | A | 550 | −8.645 | 13.431 | 13.220 | 1.00 | 17.57 |
| ATOM | 105 | CA | SER | A | 550 | −9.153 | 12.081 | 13.432 | 1.00 | 20.68 |
| ATOM | 106 | CB | SER | A | 550 | −10.343 | 11.822 | 12.512 | 1.00 | 17.19 |
| ATOM | 107 | OG | SER | A | 550 | −9.934 | 11.861 | 11.153 | 1.00 | 18.60 |
| ATOM | 108 | C | SER | A | 550 | −8.075 | 11.055 | 13.127 | 1.00 | 17.34 |
| ATOM | 109 | O | SER | A | 550 | −7.096 | 11.357 | 12.443 | 1.00 | 16.71 |
| ATOM | 110 | N | ALA | A | 551 | −8.257 | 9.841 | 13.637 | 1.00 | 18.46 |
| ATOM | 111 | CA | ALA | A | 551 | −7.304 | 8.769 | 13.380 | 1.00 | 19.34 |
| ATOM | 112 | CB | ALA | A | 551 | −7.706 | 7.507 | 14.143 | 1.00 | 17.06 |
| ATOM | 113 | C | ALA | A | 551 | −7.322 | 8.502 | 11.879 | 1.00 | 18.62 |
| ATOM | 114 | O | ALA | A | 551 | −6.284 | 8.262 | 11.261 | 1.00 | 20.73 |
| ATOM | 115 | N | GLN | A | 552 | −8.518 | 8.554 | 11.300 | 1.00 | 19.78 |
| ATOM | 116 | CA | GLN | A | 552 | −8.700 | 8.327 | 9.869 | 1.00 | 21.36 |
| ATOM | 117 | CB | GLN | A | 552 | −10.175 | 8.528 | 9.502 | 1.00 | 23.40 |
| ATOM | 118 | CG | GLN | A | 552 | −10.541 | 8.181 | 8.067 | 1.00 | 31.79 |
| ATOM | 119 | CD | GLN | A | 552 | −12.026 | 8.358 | 7.797 | 1.00 | 35.80 |
| ATOM | 120 | OE1 | GLN | A | 552 | −12.864 | 7.675 | 8.390 | 1.00 | 46.36 |
| ATOM | 121 | NE2 | GLN | A | 552 | −12.358 | 9.283 | 6.902 | 1.00 | 49.03 |
| ATOM | 122 | C | GLN | A | 552 | −7.820 | 9.301 | 9.086 | 1.00 | 21.35 |
| ATOM | 123 | O | GLN | A | 552 | −7.040 | 8.897 | 8.219 | 1.00 | 21.48 |
| ATOM | 124 | N | THR | A | 553 | −7.938 | 10.584 | 9.408 | 1.00 | 19.75 |
| ATOM | 125 | CA | THR | A | 553 | −7.151 | 11.612 | 8.739 | 1.00 | 19.52 |
| ATOM | 126 | CB | THR | A | 553 | −7.501 | 13.017 | 9.268 | 1.00 | 18.18 |
| ATOM | 127 | OG1 | THR | A | 553 | −8.871 | 13.318 | 8.971 | 1.00 | 19.96 |
| ATOM | 128 | CG2 | THR | A | 553 | −6.597 | 14.066 | 8.627 | 1.00 | 18.92 |
| ATOM | 129 | C | THR | A | 553 | −5.654 | 11.396 | 8.926 | 1.00 | 20.00 |
| ATOM | 130 | O | THR | A | 553 | −4.875 | 11.523 | 7.980 | 1.00 | 19.53 |
| ATOM | 131 | N | LEU | A | 554 | −5.260 | 11.062 | 10.150 | 1.00 | 19.10 |
| ATOM | 132 | CA | LEU | A | 554 | −3.855 | 10.853 | 10.474 | 1.00 | 20.03 |
| ATOM | 133 | CB | LEU | A | 554 | −3.650 | 10.974 | 11.987 | 1.00 | 17.91 |
| ATOM | 134 | CG | LEU | A | 554 | −4.091 | 12.323 | 12.571 | 1.00 | 18.40 |
| ATOM | 135 | CD1 | LEU | A | 554 | −3.972 | 12.316 | 14.082 | 1.00 | 16.11 |
| ATOM | 136 | CD2 | LEU | A | 554 | −3.237 | 13.436 | 11.977 | 1.00 | 23.68 |
| ATOM | 137 | C | LEU | A | 554 | −3.303 | 9.521 | 9.970 | 1.00 | 19.88 |
| ATOM | 138 | O | LEU | A | 554 | −2.093 | 9.304 | 9.994 | 1.00 | 19.74 |
| ATOM | 139 | N | LYS | A | 555 | −4.198 | 8.637 | 9.533 | 1.00 | 17.37 |
| ATOM | 140 | CA | LYS | A | 555 | −3.833 | 7.327 | 8.997 | 1.00 | 19.69 |
| ATOM | 141 | CB | LYS | A | 555 | −3.032 | 7.523 | 7.708 | 1.00 | 26.45 |
| ATOM | 142 | CG | LYS | A | 555 | −3.740 | 8.429 | 6.708 | 1.00 | 33.54 |
| ATOM | 143 | CD | LYS | A | 555 | −2.829 | 8.837 | 5.565 | 1.00 | 36.95 |
| ATOM | 144 | CE | LYS | A | 555 | −3.542 | 9.803 | 4.633 | 1.00 | 36.53 |
| ATOM | 145 | NZ | LYS | A | 555 | −2.645 | 10.308 | 3.562 | 1.00 | 43.43 |
| ATOM | 146 | C | LYS | A | 555 | −3.043 | 6.454 | 9.976 | 1.00 | 19.45 |
| ATOM | 147 | O | LYS | A | 555 | −2.294 | 5.569 | 9.565 | 1.00 | 18.29 |
| ATOM | 148 | N | ILE | A | 556 | −3.227 | 6.688 | 11.271 | 1.00 | 17.06 |
| ATOM | 149 | CA | ILE | A | 556 | −2.501 | 5.917 | 12.273 | 1.00 | 16.83 |
| ATOM | 150 | CB | ILE | A | 556 | −2.475 | 6.638 | 13.641 | 1.00 | 13.95 |
| ATOM | 151 | CG2 | ILE | A | 556 | −1.565 | 7.856 | 13.569 | 1.00 | 16.87 |
| ATOM | 152 | CG1 | ILE | A | 556 | −3.897 | 7.017 | 14.062 | 1.00 | 14.93 |
| ATOM | 153 | CD1 | ILE | A | 556 | −3.981 | 7.598 | 15.460 | 1.00 | 19.73 |
| ATOM | 154 | C | ILE | A | 556 | −3.019 | 4.496 | 12.489 | 1.00 | 14.60 |
| ATOM | 155 | O | ILE | A | 556 | −2.443 | 3.745 | 13.271 | 1.00 | 12.74 |
| ATOM | 156 | N | THR | A | 557 | −4.099 | 4.125 | 11.806 | 1.00 | 14.27 |
| ATOM | 157 | CA | THR | A | 557 | −4.653 | 2.776 | 11.938 | 1.00 | 15.05 |
| ATOM | 158 | CB | THR | A | 557 | −6.187 | 2.772 | 11.699 | 1.00 | 21.22 |
| ATOM | 159 | OG1 | THR | A | 557 | −6.813 | 3.726 | 12.567 | 1.00 | 20.77 |
| ATOM | 160 | CG2 | THR | A | 557 | −6.773 | 1.398 | 11.982 | 1.00 | 21.26 |
| ATOM | 161 | C | THR | A | 557 | −3.991 | 1.855 | 10.907 | 1.00 | 14.39 |
| ATOM | 162 | O | THR | A | 557 | −4.131 | 0.631 | 10.955 | 1.00 | 16.89 |
| ATOM | 163 | N | ASP | A | 558 | −3.257 | 2.456 | 9.979 | 1.00 | 14.56 |
| ATOM | 164 | CA | ASP | A | 558 | −2.593 | 1.702 | 8.923 | 1.00 | 14.77 |
| ATOM | 165 | CB | ASP | A | 558 | −2.374 | 2.613 | 7.713 | 1.00 | 20.77 |
| ATOM | 166 | CG | ASP | A | 558 | −3.648 | 3.312 | 7.274 | 1.00 | 30.09 |
| ATOM | 167 | OD1 | ASP | A | 558 | −4.657 | 2.616 | 7.031 | 1.00 | 38.75 |
| ATOM | 168 | OD2 | ASP | A | 558 | −3.642 | 4.555 | 7.173 | 1.00 | 46.69 |
| ATOM | 169 | C | ASP | A | 558 | −1.258 | 1.086 | 9.351 | 1.00 | 14.68 |
| ATOM | 170 | O | ASP | A | 558 | −0.365 | 1.787 | 9.829 | 1.00 | 13.87 |
| ATOM | 171 | N | PHE | A | 559 | −1.124 | −0.227 | 9.175 | 1.00 | 13.82 |
| ATOM | 172 | CA | PHE | A | 559 | 0.119 | −0.916 | 9.526 | 1.00 | 13.07 |
| ATOM | 173 | CB | PHE | A | 559 | 0.000 | −2.427 | 9.283 | 1.00 | 14.41 |
| ATOM | 174 | CG | PHE | A | 559 | −0.732 | −3.175 | 10.366 | 1.00 | 18.21 |
| ATOM | 175 | CD1 | PHE | A | 559 | −0.289 | −3.134 | 11.680 | 1.00 | 14.30 |
| ATOM | 176 | CD2 | PHE | A | 559 | −1.836 | −3.960 | 10.060 | 1.00 | 16.61 |
| ATOM | 177 | CE1 | PHE | A | 559 | −0.931 | −3.866 | 12.668 | 1.00 | 15.41 |
| ATOM | 178 | CE2 | PHE | A | 559 | −2.482 | −4.692 | 11.043 | 1.00 | 15.41 |
| ATOM | 179 | CZ | PHE | A | 559 | −2.031 | −4.647 | 12.344 | 1.00 | 12.10 |
| ATOM | 180 | C | PHE | A | 559 | 1.277 | −0.386 | 8.680 | 1.00 | 11.71 |
| ATOM | 181 | O | PHE | A | 559 | 2.431 | −0.411 | 9.109 | 1.00 | 12.25 |
| ATOM | 182 | N | SER | A | 560 | 0.965 | 0.079 | 7.473 | 1.00 | 13.82 |
| ATOM | 183 | CA | SER | A | 560 | 1.979 | 0.597 | 6.555 | 1.00 | 13.86 |
| ATOM | 184 | CB | SER | A | 560 | 1.538 | 0.384 | 5.106 | 1.00 | 19.52 |
| ATOM | 185 | OG | SER | A | 560 | 1.411 | −0.996 | 4.816 | 1.00 | 19.35 |
| ATOM | 186 | C | SER | A | 560 | 2.294 | 2.071 | 6.770 | 1.00 | 12.88 |
| ATOM | 187 | O | SER | A | 560 | 2.959 | 2.698 | 5.945 | 1.00 | 14.29 |
| ATOM | 188 | N | PHE | A | 561 | 1.815 | 2.608 | 7.886 | 1.00 | 12.82 |
| ATOM | 189 | CA | PHE | A | 561 | 2.021 | 4.009 | 8.261 | 1.00 | 13.63 |
| ATOM | 190 | CB | PHE | A | 561 | 1.567 | 4.207 | 9.713 | 1.00 | 14.59 |
| ATOM | 191 | CG | PHE | A | 561 | 1.835 | 5.583 | 10.258 | 1.00 | 12.08 |
| ATOM | 192 | CD1 | PHE | A | 561 | 0.956 | 6.625 | 10.014 | 1.00 | 17.74 |
| ATOM | 193 | CD2 | PHE | A | 561 | 2.967 | 5.829 | 11.022 | 1.00 | 10.31 |
| ATOM | 194 | CE1 | PHE | A | 561 | 1.199 | 7.891 | 10.524 | 1.00 | 21.75 |
| ATOM | 195 | CE2 | PHE | A | 561 | 3.220 | 7.093 | 11.537 | 1.00 | 13.27 |
| ATOM | 196 | CZ | PHE | A | 561 | 2.333 | 8.126 | 11.287 | 1.00 | 18.85 |
| ATOM | 197 | C | PHE | A | 561 | 3.466 | 4.498 | 8.125 | 1.00 | 14.38 |
| ATOM | 198 | O | PHE | A | 561 | 4.413 | 3.791 | 8.485 | 1.00 | 11.10 |
| ATOM | 199 | N | SER | A | 562 | 3.625 | 5.716 | 7.607 | 1.00 | 14.38 |
| ATOM | 200 | CA | SER | A | 562 | 4.939 | 6.334 | 7.453 | 1.00 | 13.72 |
| ATOM | 201 | CB | SER | A | 562 | 5.283 | 6.520 | 5.972 | 1.00 | 15.96 |
| ATOM | 202 | OG | SER | A | 562 | 6.542 | 7.159 | 5.835 | 1.00 | 23.25 |
| ATOM | 203 | C | SER | A | 562 | 4.940 | 7.695 | 8.159 | 1.00 | 12.53 |
| ATOM | 204 | O | SER | A | 562 | 4.009 | 8.490 | 8.014 | 1.00 | 12.55 |
| ATOM | 205 | N | ASP | A | 563 | 5.992 | 7.965 | 8.922 | 1.00 | 13.45 |
| ATOM | 206 | CA | ASP | A | 563 | 6.094 | 9.223 | 9.661 | 1.00 | 15.79 |
| ATOM | 207 | CB | ASP | A | 563 | 6.662 | 8.953 | 11.050 | 1.00 | 13.73 |
| ATOM | 208 | CG | ASP | A | 563 | 8.166 | 8.765 | 11.022 | 1.00 | 15.49 |
| ATOM | 209 | OD1 | ASP | A | 563 | 8.895 | 9.680 | 11.465 | 1.00 | 18.81 |
| ATOM | 210 | OD2 | ASP | A | 563 | 8.625 | 7.712 | 10.537 | 1.00 | 16.12 |
| ATOM | 211 | C | ASP | A | 563 | 7.009 | 10.232 | 8.974 | 1.00 | 16.63 |
| ATOM | 212 | O | ASP | A | 563 | 7.112 | 11.383 | 9.404 | 1.00 | 17.82 |
| ATOM | 213 | N | PHE | A | 564 | 7.673 | 9.796 | 7.911 | 1.00 | 16.51 |
| ATOM | 214 | CA | PHE | A | 564 | 8.633 | 10.635 | 7.205 | 1.00 | 18.23 |
| ATOM | 215 | CB | PHE | A | 564 | 9.207 | 9.871 | 6.011 | 1.00 | 15.87 |
| ATOM | 216 | CG | PHE | A | 564 | 10.446 | 10.494 | 5.438 | 1.00 | 26.61 |
| ATOM | 217 | CD1 | PHE | A | 564 | 11.599 | 10.592 | 6.201 | 1.00 | 20.64 |
| ATOM | 218 | CD2 | PHE | A | 564 | 10.458 | 10.991 | 4.144 | 1.00 | 27.32 |
| ATOM | 219 | CE1 | PHE | A | 564 | 12.742 | 11.174 | 5.685 | 1.00 | 30.38 |
| ATOM | 220 | CE2 | PHE | A | 564 | 11.599 | 11.574 | 3.622 | 1.00 | 33.45 |
| ATOM | 221 | CZ | PHE | A | 564 | 12.742 | 11.665 | 4.394 | 1.00 | 33.80 |
| ATOM | 222 | C | PHE | A | 564 | 8.157 | 12.010 | 6.740 | 1.00 | 18.52 |
| ATOM | 223 | O | PHE | A | 564 | 8.942 | 12.959 | 6.728 | 1.00 | 19.54 |
| ATOM | 224 | N | GLU | A | 565 | 6.885 | 12.122 | 6.369 | 1.00 | 16.14 |
| ATOM | 225 | CA | GLU | A | 565 | 6.340 | 13.391 | 5.890 | 1.00 | 19.03 |
| ATOM | 226 | CB | GLU | A | 565 | 5.277 | 13.142 | 4.813 | 1.00 | 18.54 |
| ATOM | 227 | CG | GLU | A | 565 | 5.708 | 12.238 | 3.668 | 1.00 | 33.46 |
| ATOM | 228 | CD | GLU | A | 565 | 6.004 | 10.818 | 4.117 | 1.00 | 40.92 |
| ATOM | 229 | OE1 | GLU | A | 565 | 5.205 | 10.258 | 4.899 | 1.00 | 41.02 |
| ATOM | 230 | OE2 | GLU | A | 565 | 7.032 | 10.259 | 3.680 | 1.00 | 50.71 |
| ATOM | 231 | C | GLU | A | 565 | 5.710 | 14.229 | 7.002 | 1.00 | 19.43 |
| ATOM | 232 | O | GLU | A | 565 | 5.158 | 15.301 | 6.740 | 1.00 | 20.33 |
| ATOM | 233 | N | LEU | A | 566 | 5.786 | 13.746 | 8.238 | 1.00 | 13.91 |
| ATOM | 234 | CA | LEU | A | 566 | 5.193 | 14.463 | 9.362 | 1.00 | 15.26 |
| ATOM | 235 | CB | LEU | A | 566 | 4.515 | 13.482 | 10.321 | 1.00 | 15.63 |
| ATOM | 236 | CG | LEU | A | 566 | 3.329 | 12.669 | 9.799 | 1.00 | 24.63 |
| ATOM | 237 | CD1 | LEU | A | 566 | 2.864 | 11.698 | 10.881 | 1.00 | 23.72 |
| ATOM | 238 | CD2 | LEU | A | 566 | 2.199 | 13.607 | 9.399 | 1.00 | 33.15 |
| ATOM | 239 | C | LEU | A | 566 | 6.187 | 15.306 | 10.147 | 1.00 | 14.43 |
| ATOM | 240 | O | LEU | A | 566 | 7.358 | 14.960 | 10.265 | 1.00 | 17.81 |
| ATOM | 241 | N | SER | A | 567 | 5.699 | 16.419 | 10.685 | 1.00 | 14.78 |
| ATOM | 242 | CA | SER | A | 567 | 6.517 | 17.317 | 11.486 | 1.00 | 14.98 |
| ATOM | 243 | CB | SER | A | 567 | 5.928 | 18.727 | 11.467 | 1.00 | 21.89 |
| ATOM | 244 | OG | SER | A | 567 | 4.679 | 18.752 | 12.144 | 1.00 | 17.63 |
| ATOM | 245 | C | SER | A | 567 | 6.492 | 16.797 | 12.922 | 1.00 | 13.89 |
| ATOM | 246 | O | SER | A | 567 | 5.654 | 15.963 | 13.268 | 1.00 | 13.82 |
| ATOM | 247 | N | ASP | A | 568 | 7.403 | 17.289 | 13.753 | 1.00 | 15.43 |
| ATOM | 248 | CA | ASP | A | 568 | 7.428 | 16.869 | 15.149 | 1.00 | 15.57 |
| ATOM | 249 | CB | ASP | A | 568 | 8.536 | 17.603 | 15.904 | 1.00 | 19.60 |
| ATOM | 250 | CG | ASP | A | 568 | 9.888 | 16.947 | 15.727 | 1.00 | 16.75 |
| ATOM | 251 | OD1 | ASP | A | 568 | 9.989 | 16.021 | 14.899 | 1.00 | 19.05 |
| ATOM | 252 | OD2 | ASP | A | 568 | 10.848 | 17.355 | 16.415 | 1.00 | 23.47 |
| ATOM | 253 | C | ASP | A | 568 | 6.076 | 17.145 | 15.805 | 1.00 | 16.52 |
| ATOM | 254 | O | ASP | A | 568 | 5.559 | 16.313 | 16.556 | 1.00 | 15.97 |
| ATOM | 255 | N | LEU | A | 569 | 5.505 | 18.311 | 15.516 | 1.00 | 15.09 |
| ATOM | 256 | CA | LEU | A | 569 | 4.213 | 18.683 | 16.076 | 1.00 | 15.08 |
| ATOM | 257 | CB | LEU | A | 569 | 3.815 | 20.093 | 15.620 | 1.00 | 16.05 |
| ATOM | 258 | CG | LEU | A | 569 | 2.385 | 20.516 | 15.974 | 1.00 | 23.47 |
| ATOM | 259 | CD1 | LEU | A | 569 | 2.203 | 20.527 | 17.489 | 1.00 | 21.65 |
| ATOM | 260 | CD2 | LEU | A | 569 | 2.103 | 21.896 | 15.398 | 1.00 | 27.54 |
| ATOM | 261 | C | LEU | A | 569 | 3.136 | 17.694 | 15.647 | 1.00 | 12.90 |
| ATOM | 262 | O | LEU | A | 569 | 2.279 | 17.308 | 16.441 | 1.00 | 16.26 |
| ATOM | 263 | N | GLU | A | 570 | 3.184 | 17.281 | 14.386 | 1.00 | 13.40 |
| ATOM | 264 | CA | GLU | A | 570 | 2.202 | 16.340 | 13.869 | 1.00 | 14.08 |
| ATOM | 265 | CB | GLU | A | 570 | 2.354 | 16.196 | 12.352 | 1.00 | 14.43 |
| ATOM | 266 | CG | GLU | A | 570 | 1.885 | 17.432 | 11.579 | 1.00 | 20.85 |
| ATOM | 267 | CD | GLU | A | 570 | 2.062 | 17.291 | 10.081 | 1.00 | 23.63 |
| ATOM | 268 | OE1 | GLU | A | 570 | 3.218 | 17.158 | 9.632 | 1.00 | 21.68 |
| ATOM | 269 | OE2 | GLU | A | 570 | 1.047 | 17.311 | 9.352 | 1.00 | 28.09 |
| ATOM | 270 | C | GLU | A | 570 | 2.309 | 14.978 | 14.547 | 1.00 | 14.43 |
| ATOM | 271 | O | GLU | A | 570 | 1.293 | 14.319 | 14.780 | 1.00 | 15.20 |
| ATOM | 272 | N | THR | A | 571 | 3.529 | 14.551 | 14.866 | 1.00 | 12.67 |
| ATOM | 273 | CA | THR | A | 571 | 3.694 | 13.259 | 15.538 | 1.00 | 12.83 |
| ATOM | 274 | CB | THR | A | 571 | 5.182 | 12.816 | 15.638 | 1.00 | 13.30 |
| ATOM | 275 | OG1 | THR | A | 571 | 5.915 | 13.737 | 16.452 | 1.00 | 12.68 |
| ATOM | 276 | CG2 | THR | A | 571 | 5.817 | 12.738 | 14.250 | 1.00 | 12.38 |
| ATOM | 277 | C | THR | A | 571 | 3.110 | 13.360 | 16.945 | 1.00 | 11.29 |
| ATOM | 278 | O | THR | A | 571 | 2.589 | 12.381 | 17.483 | 1.00 | 12.14 |
| ATOM | 279 | N | ALA | A | 572 | 3.202 | 14.548 | 17.538 | 1.00 | 12.42 |
| ATOM | 280 | CA | ALA | A | 572 | 2.659 | 14.780 | 18.869 | 1.00 | 11.84 |
| ATOM | 281 | CB | ALA | A | 572 | 3.090 | 16.142 | 19.388 | 1.00 | 12.18 |
| ATOM | 282 | C | ALA | A | 572 | 1.136 | 14.701 | 18.803 | 1.00 | 13.34 |
| ATOM | 283 | O | ALA | A | 572 | 0.500 | 14.128 | 19.683 | 1.00 | 11.72 |
| ATOM | 284 | N | LEU | A | 573 | 0.554 | 15.283 | 17.758 | 1.00 | 12.67 |
| ATOM | 285 | CA | LEU | A | 573 | −0.894 | 15.244 | 17.583 | 1.00 | 13.98 |
| ATOM | 286 | CB | LEU | A | 573 | −1.307 | 16.104 | 16.384 | 1.00 | 14.33 |
| ATOM | 287 | CG | LEU | A | 573 | −1.135 | 17.619 | 16.544 | 1.00 | 14.55 |
| ATOM | 288 | CD1 | LEU | A | 573 | −1.445 | 18.310 | 15.224 | 1.00 | 21.57 |
| ATOM | 289 | CD2 | LEU | A | 573 | −2.061 | 18.125 | 17.647 | 1.00 | 20.27 |
| ATOM | 290 | C | LEU | A | 573 | −1.338 | 13.795 | 17.366 | 1.00 | 15.26 |
| ATOM | 291 | O | LEU | A | 573 | −2.394 | 13.378 | 17.853 | 1.00 | 15.01 |
| ATOM | 292 | N | CYS | A | 574 | −0.534 | 13.030 | 16.632 | 1.00 | 13.44 |
| ATOM | 293 | CA | CYS | A | 574 | −0.852 | 11.625 | 16.385 | 1.00 | 13.06 |
| ATOM | 294 | CB | CYS | A | 574 | 0.187 | 10.974 | 15.470 | 1.00 | 12.27 |
| ATOM | 295 | SG | CYS | A | 574 | 0.049 | 11.382 | 13.714 | 1.00 | 15.24 |
| ATOM | 296 | C | CYS | A | 574 | −0.873 | 10.864 | 17.703 | 1.00 | 12.15 |
| ATOM | 297 | O | CYS | A | 574 | −1.710 | 9.991 | 17.919 | 1.00 | 13.51 |
| ATOM | 298 | N | THR | A | 575 | 0.066 | 11.194 | 18.581 | 1.00 | 11.60 |
| ATOM | 299 | CA | THR | A | 575 | 0.158 | 10.532 | 19.872 | 1.00 | 12.28 |
| ATOM | 300 | CB | THR | A | 575 | 1.453 | 10.949 | 20.592 | 1.00 | 10.90 |
| ATOM | 301 | OG1 | THR | A | 575 | 2.575 | 10.576 | 19.777 | 1.00 | 13.47 |
| ATOM | 302 | CG2 | THR | A | 575 | 1.565 | 10.264 | 21.944 | 1.00 | 10.06 |
| ATOM | 303 | C | THR | A | 575 | −1.068 | 10.841 | 20.726 | 1.00 | 11.74 |
| ATOM | 304 | O | THR | A | 575 | −1.613 | 9.952 | 21.379 | 1.00 | 10.78 |
| ATOM | 305 | N | ILE | A | 576 | −1.514 | 12.095 | 20.717 | 1.00 | 12.29 |
| ATOM | 306 | CA | ILE | A | 576 | −2.700 | 12.453 | 21.484 | 1.00 | 11.92 |
| ATOM | 307 | CB | ILE | A | 576 | −3.046 | 13.945 | 21.354 | 1.00 | 11.38 |
| ATOM | 308 | CG2 | ILE | A | 576 | −4.371 | 14.222 | 22.053 | 1.00 | 12.83 |
| ATOM | 309 | CG1 | ILE | A | 576 | −1.933 | 14.802 | 21.965 | 1.00 | 17.73 |
| ATOM | 310 | CD1 | ILE | A | 576 | −2.154 | 16.292 | 21.798 | 1.00 | 13.34 |
| ATOM | 311 | C | ILE | A | 576 | −3.880 | 11.636 | 20.960 | 1.00 | 12.34 |
| ATOM | 312 | O | ILE | A | 576 | −4.674 | 11.103 | 21.737 | 1.00 | 13.64 |
| ATOM | 313 | N | ARG | A | 577 | −3.985 | 11.532 | 19.638 | 1.00 | 13.14 |
| ATOM | 314 | CA | ARG | A | 577 | −5.071 | 10.772 | 19.032 | 1.00 | 13.22 |
| ATOM | 315 | CB | ARG | A | 577 | −5.029 | 10.888 | 17.503 | 1.00 | 12.21 |
| ATOM | 316 | CG | ARG | A | 577 | −6.113 | 10.084 | 16.795 | 1.00 | 12.42 |
| ATOM | 317 | CD | ARG | A | 577 | −7.492 | 10.383 | 17.365 | 1.00 | 10.79 |
| ATOM | 318 | NE | ARG | A | 577 | −7.912 | 11.757 | 17.111 | 1.00 | 18.46 |
| ATOM | 319 | CZ | ARG | A | 577 | −9.015 | 12.304 | 17.612 | 1.00 | 16.47 |
| ATOM | 320 | NH1 | ARG | A | 577 | −9.319 | 13.564 | 17.325 | 1.00 | 24.31 |
| ATOM | 321 | NH2 | ARG | A | 577 | −9.809 | 11.596 | 18.405 | 1.00 | 18.21 |
| ATOM | 322 | C | ARG | A | 577 | −5.029 | 9.304 | 19.455 | 1.00 | 12.45 |
| ATOM | 323 | O | ARG | A | 577 | −6.069 | 8.675 | 19.625 | 1.00 | 11.98 |
| ATOM | 324 | N | MET | A | 578 | −3.831 | 8.756 | 19.630 | 1.00 | 10.43 |
| ATOM | 325 | CA | MET | A | 578 | −3.716 | 7.367 | 20.058 | 1.00 | 11.28 |
| ATOM | 326 | CB | MET | A | 578 | −2.245 | 6.934 | 20.090 | 1.00 | 11.91 |
| ATOM | 327 | CG | MET | A | 578 | −1.605 | 6.808 | 18.716 | 1.00 | 12.93 |
| ATOM | 328 | SD | MET | A | 578 | 0.134 | 6.324 | 18.830 | 1.00 | 16.07 |
| ATOM | 329 | CE | MET | A | 578 | 0.781 | 7.021 | 17.300 | 1.00 | 19.76 |
| ATOM | 330 | C | MET | A | 578 | −4.332 | 7.191 | 21.445 | 1.00 | 12.18 |
| ATOM | 331 | O | MET | A | 578 | −5.103 | 6.260 | 21.675 | 1.00 | 11.26 |
| ATOM | 332 | N | PHE | A | 579 | −3.982 | 8.080 | 22.372 | 1.00 | 11.60 |
| ATOM | 333 | CA | PHE | A | 579 | −4.521 | 8.004 | 23.725 | 1.00 | 11.60 |
| ATOM | 334 | CB | PHE | A | 579 | −3.921 | 9.097 | 24.619 | 1.00 | 12.17 |
| ATOM | 335 | CG | PHE | A | 579 | −2.584 | 8.748 | 25.201 | 1.00 | 9.68 |
| ATOM | 336 | CD1 | PHE | A | 579 | −1.415 | 9.023 | 24.510 | 1.00 | 13.42 |
| ATOM | 337 | CD2 | PHE | A | 579 | −2.497 | 8.148 | 26.450 | 1.00 | 12.49 |
| ATOM | 338 | CE1 | PHE | A | 579 | −0.178 | 8.709 | 25.054 | 1.00 | 10.87 |
| ATOM | 339 | CE2 | PHE | A | 579 | −1.264 | 7.829 | 27.000 | 1.00 | 13.01 |
| ATOM | 340 | CZ | PHE | A | 579 | −0.103 | 8.113 | 26.301 | 1.00 | 12.83 |
| ATOM | 341 | C | PHE | A | 579 | −6.040 | 8.170 | 23.708 | 1.00 | 12.84 |
| ATOM | 342 | O | PHE | A | 579 | −6.765 | 7.429 | 24.367 | 1.00 | 12.73 |
| ATOM | 343 | N | THR | A | 580 | −6.504 | 9.151 | 22.943 | 1.00 | 15.04 |
| ATOM | 344 | CA | THR | A | 580 | −7.926 | 9.459 | 22.839 | 1.00 | 14.30 |
| ATOM | 345 | CB | THR | A | 580 | −8.145 | 10.702 | 21.967 | 1.00 | 10.78 |
| ATOM | 346 | OG1 | THR | A | 580 | −7.344 | 11.780 | 22.469 | 1.00 | 18.08 |
| ATOM | 347 | CG2 | THR | A | 580 | −9.610 | 11.115 | 21.987 | 1.00 | 13.16 |
| ATOM | 348 | C | THR | A | 580 | −8.772 | 8.327 | 22.273 | 1.00 | 14.34 |
| ATOM | 349 | O | THR | A | 580 | −9.774 | 7.936 | 22.871 | 1.00 | 14.67 |
| ATOM | 350 | N | ASP | A | 581 | −8.367 | 7.806 | 21.119 | 1.00 | 15.75 |
| ATOM | 351 | CA | ASP | A | 581 | −9.106 | 6.731 | 20.466 | 1.00 | 14.61 |
| ATOM | 352 | CB | ASP | A | 581 | −8.724 | 6.668 | 18.990 | 1.00 | 16.27 |
| ATOM | 353 | CG | ASP | A | 581 | −9.398 | 7.757 | 18.181 | 1.00 | 20.38 |
| ATOM | 354 | OD1 | ASP | A | 581 | −9.762 | 8.797 | 18.776 | 1.00 | 15.70 |
| ATOM | 355 | OD2 | ASP | A | 581 | −9.560 | 7.580 | 16.958 | 1.00 | 17.57 |
| ATOM | 356 | C | ASP | A | 581 | −8.970 | 5.365 | 21.123 | 1.00 | 15.19 |
| ATOM | 357 | O | ASP | A | 581 | −9.627 | 4.402 | 20.719 | 1.00 | 14.62 |
| ATOM | 358 | N | LEU | A | 582 | −8.110 | 5.275 | 22.130 | 1.00 | 13.55 |
| ATOM | 359 | CA | LEU | A | 582 | −7.957 | 4.032 | 22.867 | 1.00 | 14.36 |
| ATOM | 360 | CB | LEU | A | 582 | −6.484 | 3.739 | 23.156 | 1.00 | 16.24 |
| ATOM | 361 | CG | LEU | A | 582 | −5.770 | 3.024 | 22.006 | 1.00 | 14.56 |
| ATOM | 362 | CD1 | LEU | A | 582 | −4.272 | 2.968 | 22.250 | 1.00 | 12.82 |
| ATOM | 363 | CD2 | LEU | A | 582 | −6.351 | 1.625 | 21.865 | 1.00 | 12.05 |
| ATOM | 364 | C | LEU | A | 582 | −8.739 | 4.189 | 24.168 | 1.00 | 17.31 |
| ATOM | 365 | O | LEU | A | 582 | −8.617 | 3.378 | 25.081 | 1.00 | 17.82 |
| ATOM | 366 | N | ASN | A | 583 | −9.533 | 5.255 | 24.246 | 1.00 | 17.93 |
| ATOM | 367 | CA | ASN | A | 583 | −10.361 | 5.500 | 25.420 | 1.00 | 21.01 |
| ATOM | 368 | CB | ASN | A | 583 | −11.405 | 4.377 | 25.525 | 1.00 | 22.07 |
| ATOM | 369 | CG | ASN | A | 583 | −12.368 | 4.564 | 26.682 | 1.00 | 34.95 |
| ATOM | 370 | OD1 | ASN | A | 583 | −12.921 | 5.645 | 26.879 | 1.00 | 34.93 |
| ATOM | 371 | ND2 | ASN | A | 583 | −12.589 | 3.496 | 27.444 | 1.00 | 39.94 |
| ATOM | 372 | C | ASN | A | 583 | −9.516 | 5.575 | 26.693 | 1.00 | 23.16 |
| ATOM | 373 | O | ASN | A | 583 | −9.866 | 4.978 | 27.713 | 1.00 | 27.41 |
| ATOM | 374 | N | LEU | A | 584 | −8.406 | 6.313 | 26.634 | 1.00 | 18.24 |
| ATOM | 375 | CA | LEU | A | 584 | −7.517 | 6.446 | 27.791 | 1.00 | 18.39 |
| ATOM | 376 | CB | LEU | A | 584 | −6.060 | 6.188 | 27.379 | 1.00 | 13.74 |
| ATOM | 377 | CG | LEU | A | 584 | −5.713 | 4.823 | 26.778 | 1.00 | 13.61 |
| ATOM | 378 | CD1 | LEU | A | 584 | −4.226 | 4.789 | 26.412 | 1.00 | 16.18 |
| ATOM | 379 | CD2 | LEU | A | 584 | −6.050 | 3.726 | 27.769 | 1.00 | 15.78 |
| ATOM | 380 | C | LEU | A | 584 | −7.591 | 7.807 | 28.486 | 1.00 | 18.85 |
| ATOM | 381 | O | LEU | A | 584 | −7.377 | 7.904 | 29.695 | 1.00 | 18.54 |
| ATOM | 382 | N | VAL | A | 585 | −7.890 | 8.853 | 27.721 | 1.00 | 18.53 |
| ATOM | 383 | CA | VAL | A | 585 | −7.956 | 10.211 | 28.258 | 1.00 | 20.90 |
| ATOM | 384 | CB | VAL | A | 585 | −8.157 | 11.234 | 27.123 | 1.00 | 25.63 |
| ATOM | 385 | CG1 | VAL | A | 585 | −8.083 | 12.648 | 27.672 | 1.00 | 26.57 |
| ATOM | 386 | CG2 | VAL | A | 585 | −7.102 | 11.024 | 26.054 | 1.00 | 26.78 |
| ATOM | 387 | C | VAL | A | 585 | −9.049 | 10.416 | 29.307 | 1.00 | 21.72 |
| ATOM | 388 | O | VAL | A | 585 | −8.771 | 10.839 | 30.429 | 1.00 | 21.94 |
| ATOM | 389 | N | GLN | A | 586 | −10.291 | 10.118 | 28.945 | 1.00 | 22.79 |
| ATOM | 390 | CA | GLN | A | 586 | −11.394 | 10.290 | 29.881 | 1.00 | 25.11 |
| ATOM | 391 | CB | GLN | A | 586 | −12.728 | 10.306 | 29.131 | 1.00 | 34.29 |
| ATOM | 392 | CG | GLN | A | 586 | −13.719 | 11.317 | 29.678 | 1.00 | 39.65 |
| ATOM | 393 | CD | GLN | A | 586 | −13.155 | 12.725 | 29.663 | 1.00 | 50.17 |
| ATOM | 394 | OE1 | GLN | A | 586 | −12.715 | 13.218 | 28.622 | 1.00 | 52.20 |
| ATOM | 395 | NE2 | GLN | A | 586 | −13.160 | 13.380 | 30.820 | 1.00 | 48.68 |
| ATOM | 396 | C | GLN | A | 586 | −11.402 | 9.172 | 30.914 | 1.00 | 22.36 |
| ATOM | 397 | O | GLN | A | 586 | −11.587 | 9.413 | 32.108 | 1.00 | 23.81 |
| ATOM | 398 | N | ASN | A | 587 | −11.195 | 7.948 | 30.441 | 1.00 | 22.47 |
| ATOM | 399 | CA | ASN | A | 587 | −11.181 | 6.769 | 31.298 | 1.00 | 19.07 |
| ATOM | 400 | CB | ASN | A | 587 | −10.651 | 5.565 | 30.507 | 1.00 | 21.06 |
| ATOM | 401 | CG | ASN | A | 587 | −10.944 | 4.236 | 31.179 | 1.00 | 29.14 |
| ATOM | 402 | OD1 | ASN | A | 587 | −11.098 | 4.157 | 32.397 | 1.00 | 21.26 |
| ATOM | 403 | ND2 | ASN | A | 587 | −11.002 | 3.174 | 30.382 | 1.00 | 33.44 |
| ATOM | 404 | C | ASN | A | 587 | −10.319 | 6.981 | 32.542 | 1.00 | 20.86 |
| ATOM | 405 | O | ASN | A | 587 | −10.727 | 6.646 | 33.656 | 1.00 | 19.43 |
| ATOM | 406 | N | PHE | A | 588 | −9.134 | 7.553 | 32.357 | 1.00 | 18.57 |
| ATOM | 407 | CA | PHE | A | 588 | −8.227 | 7.753 | 33.480 | 1.00 | 18.65 |
| ATOM | 408 | CB | PHE | A | 588 | −6.880 | 7.109 | 33.145 | 1.00 | 13.88 |
| ATOM | 409 | CG | PHE | A | 588 | −6.976 | 5.624 | 32.948 | 1.00 | 14.75 |
| ATOM | 410 | CD1 | PHE | A | 588 | −7.272 | 4.791 | 34.022 | 1.00 | 13.58 |
| ATOM | 411 | CD2 | PHE | A | 588 | −6.860 | 5.065 | 31.686 | 1.00 | 12.05 |
| ATOM | 412 | CE1 | PHE | A | 588 | −7.456 | 3.431 | 33.837 | 1.00 | 13.16 |
| ATOM | 413 | CE2 | PHE | A | 588 | −7.044 | 3.704 | 31.496 | 1.00 | 10.45 |
| ATOM | 414 | CZ | PHE | A | 588 | −7.344 | 2.888 | 32.576 | 1.00 | 16.21 |
| ATOM | 415 | C | PHE | A | 588 | −8.063 | 9.190 | 33.966 | 1.00 | 20.57 |
| ATOM | 416 | O | PHE | A | 588 | −7.072 | 9.538 | 34.611 | 1.00 | 18.90 |
| ATOM | 417 | N | GLN | A | 589 | −9.052 | 10.020 | 33.662 | 1.00 | 21.41 |
| ATOM | 418 | CA | GLN | A | 589 | −9.057 | 11.410 | 34.110 | 1.00 | 23.36 |
| ATOM | 419 | CB | GLN | A | 589 | −9.213 | 11.449 | 35.634 | 1.00 | 28.73 |
| ATOM | 420 | CG | GLN | A | 589 | −10.311 | 10.551 | 36.194 | 1.00 | 40.43 |
| ATOM | 421 | CD | GLN | A | 589 | −11.701 | 11.003 | 35.797 | 1.00 | 42.17 |
| ATOM | 422 | OE1 | GLN | A | 589 | −12.058 | 12.167 | 35.971 | 1.00 | 38.12 |
| ATOM | 423 | NE2 | GLN | A | 589 | −12.498 | 10.080 | 35.270 | 1.00 | 47.35 |
| ATOM | 424 | C | GLN | A | 589 | −7.805 | 12.201 | 33.725 | 1.00 | 20.90 |
| ATOM | 425 | O | GLN | A | 589 | −7.282 | 12.972 | 34.531 | 1.00 | 21.16 |
| ATOM | 426 | N | MET | A | 590 | −7.321 | 12.019 | 32.502 | 1.00 | 22.12 |
| ATOM | 427 | CA | MET | A | 590 | −6.133 | 12.745 | 32.069 | 1.00 | 21.73 |
| ATOM | 428 | CB | MET | A | 590 | −5.463 | 12.035 | 30.893 | 1.00 | 22.93 |
| ATOM | 429 | CG | MET | A | 590 | −4.751 | 10.752 | 31.257 | 1.00 | 24.06 |
| ATOM | 430 | SD | MET | A | 590 | −3.865 | 10.087 | 29.838 | 1.00 | 19.72 |
| ATOM | 431 | CE | MET | A | 590 | −3.631 | 8.404 | 30.357 | 1.00 | 17.35 |
| ATOM | 432 | C | MET | A | 590 | −6.455 | 14.172 | 31.654 | 1.00 | 22.68 |
| ATOM | 433 | O | MET | A | 590 | −7.295 | 14.398 | 30.784 | 1.00 | 23.69 |
| ATOM | 434 | N | LYS | A | 591 | −5.792 | 15.138 | 32.278 | 1.00 | 20.35 |
| ATOM | 435 | CA | LYS | A | 591 | −6.013 | 16.530 | 31.919 | 1.00 | 20.85 |
| ATOM | 436 | CB | LYS | A | 591 | −5.554 | 17.460 | 33.045 | 1.00 | 22.95 |
| ATOM | 437 | CG | LYS | A | 591 | −6.472 | 17.422 | 34.262 | 1.00 | 28.61 |
| ATOM | 438 | CD | LYS | A | 591 | −6.065 | 18.438 | 35.315 | 1.00 | 37.59 |
| ATOM | 439 | CE | LYS | A | 591 | −7.106 | 18.527 | 36.423 | 1.00 | 42.92 |
| ATOM | 440 | NZ | LYS | A | 591 | −8.417 | 19.026 | 35.915 | 1.00 | 51.43 |
| ATOM | 441 | C | LYS | A | 591 | −5.235 | 16.800 | 30.636 | 1.00 | 20.72 |
| ATOM | 442 | O | LYS | A | 591 | −4.048 | 16.486 | 30.537 | 1.00 | 21.29 |
| ATOM | 443 | N | HIS | A | 592 | −5.920 | 17.375 | 29.654 | 1.00 | 20.84 |
| ATOM | 444 | CA | HIS | A | 592 | −5.333 | 17.670 | 28.351 | 1.00 | 21.11 |
| ATOM | 445 | CB | HIS | A | 592 | −6.273 | 18.577 | 27.553 | 1.00 | 21.39 |
| ATOM | 446 | CG | HIS | A | 592 | −5.884 | 18.737 | 26.125 | 1.00 | 18.05 |
| ATOM | 447 | CD2 | HIS | A | 592 | −5.427 | 19.798 | 25.424 | 1.00 | 24.75 |
| ATOM | 448 | ND1 | HIS | A | 592 | −5.926 | 17.685 | 25.213 | 1.00 | 22.74 |
| ATOM | 449 | CE1 | HIS | A | 592 | −5.518 | 18.102 | 24.044 | 1.00 | 29.20 |
| ATOM | 450 | NE2 | HIS | A | 592 | −5.204 | 19.393 | 24.135 | 1.00 | 27.94 |
| ATOM | 451 | C | HIS | A | 592 | −3.937 | 18.295 | 28.377 | 1.00 | 20.49 |
| ATOM | 452 | O | HIS | A | 592 | −3.034 | 17.848 | 27.661 | 1.00 | 19.49 |
| ATOM | 453 | N | GLU | A | 593 | −3.765 | 19.338 | 29.184 | 1.00 | 19.12 |
| ATOM | 454 | CA | GLU | A | 593 | −2.481 | 20.019 | 29.279 | 1.00 | 18.26 |
| ATOM | 455 | CB | GLU | A | 593 | −2.592 | 21.243 | 30.192 | 1.00 | 23.02 |
| ATOM | 456 | CG | GLU | A | 593 | −1.267 | 21.957 | 30.416 | 1.00 | 32.52 |
| ATOM | 457 | CD | GLU | A | 593 | −1.422 | 23.296 | 31.116 | 1.00 | 44.01 |
| ATOM | 458 | OE1 | GLU | A | 593 | −2.062 | 24.200 | 30.535 | 1.00 | 41.08 |
| ATOM | 459 | OE2 | GLU | A | 593 | −0.901 | 23.446 | 32.244 | 1.00 | 45.85 |
| ATOM | 460 | C | GLU | A | 593 | −1.381 | 19.099 | 29.793 | 1.00 | 18.42 |
| ATOM | 461 | O | GLU | A | 593 | −0.230 | 19.195 | 29.365 | 1.00 | 16.87 |
| ATOM | 462 | N | VAL | A | 594 | −1.738 | 18.209 | 30.710 | 1.00 | 18.22 |
| ATOM | 463 | CA | VAL | A | 594 | −0.770 | 17.275 | 31.277 | 1.00 | 17.13 |
| ATOM | 464 | CB | VAL | A | 594 | −1.372 | 16.506 | 32.465 | 1.00 | 16.16 |
| ATOM | 465 | CG1 | VAL | A | 594 | −0.314 | 15.605 | 33.085 | 1.00 | 20.37 |
| ATOM | 466 | CG2 | VAL | A | 594 | −1.911 | 17.481 | 33.491 | 1.00 | 22.54 |
| ATOM | 467 | C | VAL | A | 594 | −0.299 | 16.263 | 30.235 | 1.00 | 15.25 |
| ATOM | 468 | O | VAL | A | 594 | 0.902 | 16.005 | 30.101 | 1.00 | 16.26 |
| ATOM | 469 | N | LEU | A | 595 | −1.246 | 15.681 | 29.504 | 1.00 | 13.83 |
| ATOM | 470 | CA | LEU | A | 595 | −0.897 | 14.705 | 28.478 | 1.00 | 14.01 |
| ATOM | 471 | CB | LEU | A | 595 | −2.163 | 14.147 | 27.818 | 1.00 | 9.50 |
| ATOM | 472 | CG | LEU | A | 595 | −1.969 | 13.167 | 26.653 | 1.00 | 18.45 |
| ATOM | 473 | CD1 | LEU | A | 595 | −1.068 | 12.014 | 27.076 | 1.00 | 12.38 |
| ATOM | 474 | CD2 | LEU | A | 595 | −3.318 | 12.643 | 26.201 | 1.00 | 14.12 |
| ATOM | 475 | C | LEU | A | 595 | −0.002 | 15.372 | 27.438 | 1.00 | 13.61 |
| ATOM | 476 | O | LEU | A | 595 | 1.004 | 14.804 | 27.010 | 1.00 | 13.98 |
| ATOM | 477 | N | CYS | A | 596 | −0.358 | 16.588 | 27.037 | 1.00 | 14.70 |
| ATOM | 478 | CA | CYS | A | 596 | 0.448 | 17.303 | 26.058 | 1.00 | 12.24 |
| ATOM | 479 | CB | CYS | A | 596 | −0.191 | 18.649 | 25.709 | 1.00 | 15.21 |
| ATOM | 480 | SG | CYS | A | 596 | −1.702 | 18.507 | 24.749 | 1.00 | 17.45 |
| ATOM | 481 | C | CYS | A | 596 | 1.861 | 17.534 | 26.586 | 1.00 | 12.70 |
| ATOM | 482 | O | CYS | A | 596 | 2.839 | 17.327 | 25.868 | 1.00 | 11.06 |
| ATOM | 483 | N | ARG | A | 597 | 1.970 | 17.959 | 27.841 | 1.00 | 11.36 |
| ATOM | 484 | CA | ARG | A | 597 | 3.283 | 18.211 | 28.420 | 1.00 | 11.51 |
| ATOM | 485 | CB | ARG | A | 597 | 3.141 | 18.836 | 29.815 | 1.00 | 14.74 |
| ATOM | 486 | CG | ARG | A | 597 | 4.463 | 19.343 | 30.388 | 1.00 | 15.86 |
| ATOM | 487 | CD | ARG | A | 597 | 4.294 | 20.067 | 31.727 | 1.00 | 20.22 |
| ATOM | 488 | NE | ARG | A | 597 | 3.565 | 21.332 | 31.614 | 1.00 | 21.93 |
| ATOM | 489 | CZ | ARG | A | 597 | 2.279 | 21.493 | 31.916 | 1.00 | 27.00 |
| ATOM | 490 | NH1 | ARG | A | 597 | 1.560 | 20.469 | 32.357 | 1.00 | 22.77 |
| ATOM | 491 | NH2 | ARG | A | 597 | 1.708 | 22.683 | 31.782 | 1.00 | 30.27 |
| ATOM | 492 | C | ARG | A | 597 | 4.100 | 16.919 | 28.494 | 1.00 | 11.80 |
| ATOM | 493 | O | ARG | A | 597 | 5.287 | 16.909 | 28.162 | 1.00 | 10.48 |
| ATOM | 494 | N | TRP | A | 598 | 3.454 | 15.830 | 28.905 | 1.00 | 11.97 |
| ATOM | 495 | CA | TRP | A | 598 | 4.112 | 14.530 | 29.022 | 1.00 | 9.62 |
| ATOM | 496 | CB | TRP | A | 598 | 3.125 | 13.488 | 29.556 | 1.00 | 11.57 |
| ATOM | 497 | CG | TRP | A | 598 | 3.717 | 12.110 | 29.702 | 1.00 | 10.65 |
| ATOM | 498 | CD2 | TRP | A | 598 | 3.609 | 11.028 | 28.771 | 1.00 | 9.66 |
| ATOM | 499 | CE2 | TRP | A | 598 | 4.350 | 9.948 | 29.298 | 1.00 | 8.24 |
| ATOM | 500 | CE3 | TRP | A | 598 | 2.964 | 10.866 | 27.541 | 1.00 | 11.87 |
| ATOM | 501 | CD1 | TRP | A | 598 | 4.494 | 11.656 | 30.728 | 1.00 | 9.74 |
| ATOM | 502 | NE1 | TRP | A | 598 | 4.878 | 10.359 | 30.492 | 1.00 | 11.42 |
| ATOM | 503 | CZ2 | TRP | A | 598 | 4.459 | 8.720 | 28.635 | 1.00 | 7.38 |
| ATOM | 504 | CZ3 | TRP | A | 598 | 3.075 | 9.646 | 26.886 | 1.00 | 9.43 |
| ATOM | 505 | CH2 | TRP | A | 598 | 3.818 | 8.593 | 27.434 | 1.00 | 10.02 |
| ATOM | 506 | C | TRP | A | 598 | 4.656 | 14.064 | 27.670 | 1.00 | 12.66 |
| ATOM | 507 | O | TRP | A | 598 | 5.804 | 13.617 | 27.568 | 1.00 | 11.33 |
| ATOM | 508 | N | ILE | A | 599 | 3.823 | 14.160 | 26.637 | 1.00 | 9.84 |
| ATOM | 509 | CA | ILE | A | 599 | 4.232 | 13.748 | 25.298 | 1.00 | 12.03 |
| ATOM | 510 | CB | ILE | A | 599 | 3.101 | 13.974 | 24.270 | 1.00 | 11.30 |
| ATOM | 511 | CG2 | ILE | A | 599 | 3.648 | 13.807 | 22.856 | 1.00 | 13.41 |
| ATOM | 512 | CG1 | ILE | A | 599 | 1.953 | 13.000 | 24.546 | 1.00 | 13.16 |
| ATOM | 513 | CD1 | ILE | A | 599 | 0.732 | 13.218 | 23.668 | 1.00 | 13.06 |
| ATOM | 514 | C | ILE | A | 599 | 5.460 | 14.533 | 24.855 | 1.00 | 14.21 |
| ATOM | 515 | O | ILE | A | 599 | 6.414 | 13.969 | 24.308 | 1.00 | 11.47 |
| ATOM | 516 | N | LEU | A | 600 | 5.439 | 15.839 | 25.094 | 1.00 | 14.33 |
| ATOM | 517 | CA | LEU | A | 600 | 6.566 | 16.670 | 24.707 | 1.00 | 13.61 |
| ATOM | 518 | CB | LEU | A | 600 | 6.214 | 18.151 | 24.882 | 1.00 | 13.53 |
| ATOM | 519 | CG | LEU | A | 600 | 5.073 | 18.621 | 23.971 | 1.00 | 12.80 |
| ATOM | 520 | CD1 | LEU | A | 600 | 4.652 | 20.036 | 24.336 | 1.00 | 15.66 |
| ATOM | 521 | CD2 | LEU | A | 600 | 5.518 | 18.557 | 22.520 | 1.00 | 13.09 |
| ATOM | 522 | C | LEU | A | 600 | 7.800 | 16.297 | 25.523 | 1.00 | 10.78 |
| ATOM | 523 | O | LEU | A | 600 | 8.917 | 16.324 | 25.011 | 1.00 | 10.47 |
| ATOM | 524 | N | SER | A | 601 | 7.601 | 15.925 | 26.788 | 1.00 | 10.50 |
| ATOM | 525 | CA | SER | A | 601 | 8.734 | 15.540 | 27.624 | 1.00 | 8.54 |
| ATOM | 526 | CB | SER | A | 601 | 8.303 | 15.398 | 29.085 | 1.00 | 9.00 |
| ATOM | 527 | OG | SER | A | 601 | 8.036 | 16.678 | 29.634 | 1.00 | 9.87 |
| ATOM | 528 | C | SER | A | 601 | 9.342 | 14.239 | 27.129 | 1.00 | 9.99 |
| ATOM | 529 | O | SER | A | 601 | 10.563 | 14.082 | 27.104 | 1.00 | 10.43 |
| ATOM | 530 | N | VAL | A | 602 | 8.487 | 13.300 | 26.739 | 1.00 | 10.77 |
| ATOM | 531 | CA | VAL | A | 602 | 8.968 | 12.024 | 26.223 | 1.00 | 10.98 |
| ATOM | 532 | CB | VAL | A | 602 | 7.791 | 11.081 | 25.874 | 1.00 | 12.52 |
| ATOM | 533 | CG1 | VAL | A | 602 | 8.303 | 9.856 | 25.138 | 1.00 | 9.22 |
| ATOM | 534 | CG2 | VAL | A | 602 | 7.068 | 10.658 | 27.152 | 1.00 | 11.71 |
| ATOM | 535 | C | VAL | A | 602 | 9.794 | 12.290 | 24.967 | 1.00 | 12.57 |
| ATOM | 536 | O | VAL | A | 602 | 10.935 | 11.848 | 24.855 | 1.00 | 11.99 |
| ATOM | 537 | N | LYS | A | 603 | 9.220 | 13.043 | 24.034 | 1.00 | 12.97 |
| ATOM | 538 | CA | LYS | A | 603 | 9.906 | 13.363 | 22.792 | 1.00 | 11.73 |
| ATOM | 539 | CB | LYS | A | 603 | 9.025 | 14.268 | 21.928 | 1.00 | 14.00 |
| ATOM | 540 | CG | LYS | A | 603 | 9.666 | 14.688 | 20.620 | 1.00 | 14.56 |
| ATOM | 541 | CD | LYS | A | 603 | 8.656 | 15.317 | 19.686 | 1.00 | 16.82 |
| ATOM | 542 | CE | LYS | A | 603 | 8.088 | 16.603 | 20.262 | 1.00 | 24.97 |
| ATOM | 543 | NZ | LYS | A | 603 | 7.169 | 17.256 | 19.300 | 1.00 | 39.23 |
| ATOM | 544 | C | LYS | A | 603 | 11.256 | 14.035 | 23.042 | 1.00 | 11.50 |
| ATOM | 545 | O | LYS | A | 603 | 12.252 | 13.694 | 22.414 | 1.00 | 14.04 |
| ATOM | 546 | N | LYS | A | 604 | 11.279 | 14.985 | 23.968 | 1.00 | 11.12 |
| ATOM | 547 | CA | LYS | A | 604 | 12.501 | 15.714 | 24.293 | 1.00 | 13.84 |
| ATOM | 548 | CB | LYS | A | 604 | 12.181 | 16.849 | 25.274 | 1.00 | 16.59 |
| ATOM | 549 | CG | LYS | A | 604 | 13.403 | 17.589 | 25.788 | 1.00 | 18.98 |
| ATOM | 550 | CD | LYS | A | 604 | 13.016 | 18.673 | 26.784 | 1.00 | 25.53 |
| ATOM | 551 | CE | LYS | A | 604 | 14.251 | 19.335 | 27.391 | 1.00 | 31.51 |
| ATOM | 552 | NZ | LYS | A | 604 | 13.893 | 20.351 | 28.424 | 1.00 | 29.80 |
| ATOM | 553 | C | LYS | A | 604 | 13.582 | 14.824 | 24.890 | 1.00 | 12.98 |
| ATOM | 554 | O | LYS | A | 604 | 14.776 | 15.073 | 24.710 | 1.00 | 13.10 |
| ATOM | 555 | N | ASN | A | 605 | 13.173 | 13.773 | 25.587 | 1.00 | 12.62 |
| ATOM | 556 | CA | ASN | A | 605 | 14.146 | 12.901 | 26.220 | 1.00 | 13.04 |
| ATOM | 557 | CB | ASN | A | 605 | 13.555 | 12.336 | 27.507 | 1.00 | 13.13 |
| ATOM | 558 | CG | ASN | A | 605 | 13.579 | 13.356 | 28.638 | 1.00 | 32.58 |
| ATOM | 559 | OD1 | ASN | A | 605 | 14.638 | 13.650 | 29.197 | 1.00 | 25.76 |
| ATOM | 560 | ND2 | ASN | A | 605 | 12.418 | 13.918 | 28.962 | 1.00 | 30.95 |
| ATOM | 561 | C | ASN | A | 605 | 14.757 | 11.809 | 25.352 | 1.00 | 15.12 |
| ATOM | 562 | O | ASN | A | 605 | 15.342 | 10.848 | 25.858 | 1.00 | 18.48 |
| ATOM | 563 | N | TYR | A | 606 | 14.612 | 11.961 | 24.042 | 1.00 | 10.60 |
| ATOM | 564 | CA | TYR | A | 606 | 15.233 | 11.051 | 23.093 | 1.00 | 10.45 |
| ATOM | 565 | CB | TYR | A | 606 | 14.254 | 10.645 | 21.982 | 1.00 | 10.34 |
| ATOM | 566 | CG | TYR | A | 606 | 13.420 | 9.444 | 22.358 | 1.00 | 11.02 |
| ATOM | 567 | CD1 | TYR | A | 606 | 13.950 | 8.156 | 22.297 | 1.00 | 8.31 |
| ATOM | 568 | CE1 | TYR | A | 606 | 13.222 | 7.061 | 22.752 | 1.00 | 8.28 |
| ATOM | 569 | CD2 | TYR | A | 606 | 12.139 | 9.601 | 22.874 | 1.00 | 12.47 |
| ATOM | 570 | CE2 | TYR | A | 606 | 11.410 | 8.516 | 23.335 | 1.00 | 15.05 |
| ATOM | 571 | CZ | TYR | A | 606 | 11.955 | 7.252 | 23.275 | 1.00 | 15.46 |
| ATOM | 572 | OH | TYR | A | 606 | 11.227 | 6.188 | 23.773 | 1.00 | 12.36 |
| ATOM | 573 | C | TYR | A | 606 | 16.362 | 11.908 | 22.539 | 1.00 | 11.41 |
| ATOM | 574 | O | TYR | A | 606 | 16.216 | 13.129 | 22.428 | 1.00 | 14.91 |
| ATOM | 575 | N | ARG | A | 607 | 17.499 | 11.293 | 22.234 | 1.00 | 11.41 |
| ATOM | 576 | CA | ARG | A | 607 | 18.627 | 12.042 | 21.700 | 1.00 | 10.90 |
| ATOM | 577 | CB | ARG | A | 607 | 19.933 | 11.322 | 22.034 | 1.00 | 12.68 |
| ATOM | 578 | CG | ARG | A | 607 | 20.078 | 11.019 | 23.520 | 1.00 | 10.36 |
| ATOM | 579 | CD | ARG | A | 607 | 21.460 | 10.489 | 23.870 | 1.00 | 13.25 |
| ATOM | 580 | NE | ARG | A | 607 | 21.492 | 9.929 | 25.221 | 1.00 | 9.55 |
| ATOM | 581 | CZ | ARG | A | 607 | 22.601 | 9.556 | 25.854 | 1.00 | 18.33 |
| ATOM | 582 | NH1 | ARG | A | 607 | 23.781 | 9.686 | 25.262 | 1.00 | 13.50 |
| ATOM | 583 | NH2 | ARG | A | 607 | 22.530 | 9.045 | 27.077 | 1.00 | 17.68 |
| ATOM | 584 | C | ARG | A | 607 | 18.425 | 12.150 | 20.194 | 1.00 | 10.45 |
| ATOM | 585 | O | ARG | A | 607 | 18.616 | 11.182 | 19.458 | 1.00 | 12.53 |
| ATOM | 586 | N | LYS | A | 608 | 18.052 | 13.340 | 19.738 | 1.00 | 11.88 |
| ATOM | 587 | CA | LYS | A | 608 | 17.755 | 13.555 | 18.323 | 1.00 | 13.33 |
| ATOM | 588 | CB | LYS | A | 608 | 17.273 | 14.993 | 18.104 | 1.00 | 18.33 |
| ATOM | 589 | CG | LYS | A | 608 | 18.226 | 16.064 | 18.574 | 1.00 | 34.41 |
| ATOM | 590 | CD | LYS | A | 608 | 17.558 | 17.429 | 18.538 | 1.00 | 39.16 |
| ATOM | 591 | CE | LYS | A | 608 | 18.430 | 18.485 | 19.193 | 1.00 | 44.56 |
| ATOM | 592 | NZ | LYS | A | 608 | 19.755 | 18.595 | 18.525 | 1.00 | 49.34 |
| ATOM | 593 | C | LYS | A | 608 | 18.837 | 13.220 | 17.311 | 1.00 | 13.41 |
| ATOM | 594 | O | LYS | A | 608 | 18.527 | 12.904 | 16.164 | 1.00 | 14.71 |
| ATOM | 595 | N | ASN | A | 609 | 20.098 | 13.272 | 17.724 | 1.00 | 12.65 |
| ATOM | 596 | CA | ASN | A | 609 | 21.185 | 12.977 | 16.804 | 1.00 | 15.37 |
| ATOM | 597 | CB | ASN | A | 609 | 22.383 | 13.872 | 17.121 | 1.00 | 19.55 |
| ATOM | 598 | CG | ASN | A | 609 | 22.059 | 15.347 | 16.952 | 1.00 | 21.29 |
| ATOM | 599 | OD1 | ASN | A | 609 | 21.630 | 15.779 | 15.882 | 1.00 | 28.72 |
| ATOM | 600 | ND2 | ASN | A | 609 | 22.261 | 16.126 | 18.009 | 1.00 | 36.76 |
| ATOM | 601 | C | ASN | A | 609 | 21.593 | 11.507 | 16.743 | 1.00 | 14.47 |
| ATOM | 602 | O | ASN | A | 609 | 22.486 | 11.135 | 15.981 | 1.00 | 15.29 |
| ATOM | 603 | N | VAL | A | 610 | 20.955 | 10.670 | 17.557 | 1.00 | 11.89 |
| ATOM | 604 | CA | VAL | A | 610 | 21.221 | 9.233 | 17.511 | 1.00 | 11.43 |
| ATOM | 605 | CB | VAL | A | 610 | 20.696 | 8.531 | 18.777 | 1.00 | 11.38 |
| ATOM | 606 | CG1 | VAL | A | 610 | 20.649 | 7.019 | 18.571 | 1.00 | 10.56 |
| ATOM | 607 | CG2 | VAL | A | 610 | 21.608 | 8.882 | 19.955 | 1.00 | 10.47 |
| ATOM | 608 | C | VAL | A | 610 | 20.432 | 8.809 | 16.272 | 1.00 | 12.97 |
| ATOM | 609 | O | VAL | A | 610 | 19.221 | 9.025 | 16.198 | 1.00 | 11.59 |
| ATOM | 610 | N | ALA | A | 611 | 21.131 | 8.223 | 15.302 | 1.00 | 11.37 |
| ATOM | 611 | CA | ALA | A | 611 | 20.554 | 7.833 | 14.018 | 1.00 | 12.77 |
| ATOM | 612 | CB | ALA | A | 611 | 21.579 | 7.021 | 13.223 | 1.00 | 12.83 |
| ATOM | 613 | C | ALA | A | 611 | 19.208 | 7.117 | 13.981 | 1.00 | 12.88 |
| ATOM | 614 | O | ALA | A | 611 | 18.305 | 7.522 | 13.247 | 1.00 | 14.79 |
| ATOM | 615 | N | TYR | A | 612 | 19.071 | 6.050 | 14.756 | 1.00 | 12.18 |
| ATOM | 616 | CA | TYR | A | 612 | 17.830 | 5.288 | 14.743 | 1.00 | 10.69 |
| ATOM | 617 | CB | TYR | A | 612 | 18.147 | 3.824 | 14.408 | 1.00 | 13.15 |
| ATOM | 618 | CG | TYR | A | 612 | 16.968 | 2.877 | 14.509 | 1.00 | 12.63 |
| ATOM | 619 | CD1 | TYR | A | 612 | 15.832 | 3.054 | 13.729 | 1.00 | 19.45 |
| ATOM | 620 | CE1 | TYR | A | 612 | 14.752 | 2.185 | 13.833 | 1.00 | 24.00 |
| ATOM | 621 | CD2 | TYR | A | 612 | 16.996 | 1.805 | 15.392 | 1.00 | 13.47 |
| ATOM | 622 | CE2 | TYR | A | 612 | 15.930 | 0.938 | 15.503 | 1.00 | 24.95 |
| ATOM | 623 | CZ | TYR | A | 612 | 14.812 | 1.129 | 14.722 | 1.00 | 24.27 |
| ATOM | 624 | OH | TYR | A | 612 | 13.761 | 0.245 | 14.831 | 1.00 | 25.94 |
| ATOM | 625 | C | TYR | A | 612 | 17.017 | 5.371 | 16.028 | 1.00 | 11.05 |
| ATOM | 626 | O | TYR | A | 612 | 15.812 | 5.632 | 15.989 | 1.00 | 9.25 |
| ATOM | 627 | N | HIS | A | 613 | 17.667 | 5.159 | 17.166 | 1.00 | 10.71 |
| ATOM | 628 | CA | HIS | A | 613 | 16.951 | 5.199 | 18.432 | 1.00 | 9.46 |
| ATOM | 629 | CB | HIS | A | 613 | 17.694 | 4.391 | 19.499 | 1.00 | 12.29 |
| ATOM | 630 | CG | HIS | A | 613 | 17.706 | 2.923 | 19.232 | 1.00 | 13.22 |
| ATOM | 631 | CD2 | HIS | A | 613 | 16.742 | 1.983 | 19.364 | 1.00 | 13.53 |
| ATOM | 632 | ND1 | HIS | A | 613 | 18.811 | 2.262 | 18.726 | 1.00 | 8.83 |
| ATOM | 633 | CE1 | HIS | A | 613 | 18.523 | 0.989 | 18.562 | 1.00 | 12.86 |
| ATOM | 634 | NE2 | HIS | A | 613 | 17.268 | 0.789 | 18.942 | 1.00 | 16.65 |
| ATOM | 635 | C | HIS | A | 613 | 16.686 | 6.606 | 18.936 | 1.00 | 11.81 |
| ATOM | 636 | O | HIS | A | 613 | 17.226 | 7.023 | 19.957 | 1.00 | 11.26 |
| ATOM | 637 | N | ASN | A | 614 | 15.859 | 7.333 | 18.192 | 1.00 | 12.20 |
| ATOM | 638 | CA | ASN | A | 614 | 15.459 | 8.685 | 18.556 | 1.00 | 11.65 |
| ATOM | 639 | CB | ASN | A | 614 | 15.955 | 9.702 | 17.516 | 1.00 | 19.08 |
| ATOM | 640 | CG | ASN | A | 614 | 15.631 | 9.295 | 16.090 | 1.00 | 17.63 |
| ATOM | 641 | OD1 | ASN | A | 614 | 16.529 | 9.151 | 15.253 | 1.00 | 16.40 |
| ATOM | 642 | ND2 | ASN | A | 614 | 14.349 | 9.115 | 15.802 | 1.00 | 11.39 |
| ATOM | 643 | C | ASN | A | 614 | 13.933 | 8.690 | 18.644 | 1.00 | 11.53 |
| ATOM | 644 | O | ASN | A | 614 | 13.298 | 7.647 | 18.493 | 1.00 | 9.32 |
| ATOM | 645 | N | TRP | A | 615 | 13.342 | 9.850 | 18.890 | 1.00 | 8.95 |
| ATOM | 646 | CA | TRP | A | 615 | 11.893 | 9.929 | 19.015 | 1.00 | 9.02 |
| ATOM | 647 | CB | TRP | A | 615 | 11.457 | 11.397 | 19.127 | 1.00 | 9.00 |
| ATOM | 648 | CG | TRP | A | 615 | 10.002 | 11.638 | 18.809 | 1.00 | 8.75 |
| ATOM | 649 | CD2 | TRP | A | 615 | 8.860 | 11.228 | 19.581 | 1.00 | 10.58 |
| ATOM | 650 | CE2 | TRP | A | 615 | 7.709 | 11.655 | 18.883 | 1.00 | 12.68 |
| ATOM | 651 | CE3 | TRP | A | 615 | 8.698 | 10.542 | 20.789 | 1.00 | 12.70 |
| ATOM | 652 | CD1 | TRP | A | 615 | 9.504 | 12.279 | 17.709 | 1.00 | 11.21 |
| ATOM | 653 | NE1 | TRP | A | 615 | 8.132 | 12.292 | 17.747 | 1.00 | 11.25 |
| ATOM | 654 | CZ2 | TRP | A | 615 | 6.414 | 11.418 | 19.354 | 1.00 | 12.28 |
| ATOM | 655 | CZ3 | TRP | A | 615 | 7.410 | 10.307 | 21.255 | 1.00 | 18.15 |
| ATOM | 656 | CH2 | TRP | A | 615 | 6.286 | 10.744 | 20.536 | 1.00 | 13.05 |
| ATOM | 657 | C | TRP | A | 615 | 11.090 | 9.237 | 17.910 | 1.00 | 8.08 |
| ATOM | 658 | O | TRP | A | 615 | 10.093 | 8.579 | 18.197 | 1.00 | 9.24 |
| ATOM | 659 | N | ARG | A | 616 | 11.514 | 9.365 | 16.656 | 1.00 | 9.61 |
| ATOM | 660 | CA | ARG | A | 616 | 10.750 | 8.759 | 15.570 | 1.00 | 11.24 |
| ATOM | 661 | CB | ARG | A | 616 | 11.346 | 9.129 | 14.206 | 1.00 | 9.33 |
| ATOM | 662 | CG | ARG | A | 616 | 11.241 | 10.630 | 13.885 | 1.00 | 13.33 |
| ATOM | 663 | CD | ARG | A | 616 | 9.817 | 11.169 | 14.120 | 1.00 | 11.22 |
| ATOM | 664 | NE | ARG | A | 616 | 9.705 | 12.598 | 13.823 | 1.00 | 13.61 |
| ATOM | 665 | CZ | ARG | A | 616 | 9.119 | 13.103 | 12.741 | 1.00 | 15.86 |
| ATOM | 666 | NH1 | ARG | A | 616 | 8.577 | 12.300 | 11.834 | 1.00 | 15.89 |
| ATOM | 667 | NH2 | ARG | A | 616 | 9.073 | 14.419 | 12.564 | 1.00 | 19.53 |
| ATOM | 668 | C | ARG | A | 616 | 10.607 | 7.250 | 15.705 | 1.00 | 9.88 |
| ATOM | 669 | O | ARG | A | 616 | 9.575 | 6.692 | 15.328 | 1.00 | 10.40 |
| ATOM | 670 | N | HIS | A | 617 | 11.625 | 6.582 | 16.242 | 1.00 | 7.87 |
| ATOM | 671 | CA | HIS | A | 617 | 11.514 | 5.141 | 16.419 | 1.00 | 7.37 |
| ATOM | 672 | CB | HIS | A | 617 | 12.862 | 4.523 | 16.782 | 1.00 | 11.98 |
| ATOM | 673 | CG | HIS | A | 617 | 12.742 | 3.163 | 17.390 | 1.00 | 8.70 |
| ATOM | 674 | CD2 | HIS | A | 617 | 13.043 | 2.715 | 18.632 | 1.00 | 9.51 |
| ATOM | 675 | ND1 | HIS | A | 617 | 12.206 | 2.088 | 16.713 | 1.00 | 8.99 |
| ATOM | 676 | CE1 | HIS | A | 617 | 12.183 | 1.036 | 17.510 | 1.00 | 11.83 |
| ATOM | 677 | NE2 | HIS | A | 617 | 12.684 | 1.391 | 18.681 | 1.00 | 10.58 |
| ATOM | 678 | C | HIS | A | 617 | 10.488 | 4.816 | 17.506 | 1.00 | 8.63 |
| ATOM | 679 | O | HIS | A | 617 | 9.692 | 3.888 | 17.361 | 1.00 | 10.24 |
| ATOM | 680 | N | ALA | A | 618 | 10.498 | 5.586 | 18.591 | 1.00 | 7.45 |
| ATOM | 681 | CA | ALA | A | 618 | 9.549 | 5.358 | 19.687 | 1.00 | 8.54 |
| ATOM | 682 | CB | ALA | A | 618 | 9.887 | 6.261 | 20.864 | 1.00 | 11.31 |
| ATOM | 683 | C | ALA | A | 618 | 8.126 | 5.637 | 19.210 | 1.00 | 8.39 |
| ATOM | 684 | O | ALA | A | 618 | 7.184 | 4.900 | 19.523 | 1.00 | 9.03 |
| ATOM | 685 | N | PHE | A | 619 | 7.981 | 6.725 | 18.465 | 1.00 | 8.88 |
| ATOM | 686 | CA | PHE | A | 619 | 6.695 | 7.118 | 17.911 | 1.00 | 9.63 |
| ATOM | 687 | CB | PHE | A | 619 | 6.850 | 8.418 | 17.119 | 1.00 | 12.92 |
| ATOM | 688 | CG | PHE | A | 619 | 5.618 | 8.809 | 16.357 | 1.00 | 10.44 |
| ATOM | 689 | CD1 | PHE | A | 619 | 4.432 | 9.066 | 17.021 | 1.00 | 18.21 |
| ATOM | 690 | CD2 | PHE | A | 619 | 5.646 | 8.914 | 14.974 | 1.00 | 12.41 |
| ATOM | 691 | CE1 | PHE | A | 619 | 3.291 | 9.412 | 16.324 | 1.00 | 14.74 |
| ATOM | 692 | CE2 | PHE | A | 619 | 4.508 | 9.260 | 14.270 | 1.00 | 13.16 |
| ATOM | 693 | CZ | PHE | A | 619 | 3.328 | 9.512 | 14.947 | 1.00 | 11.01 |
| ATOM | 694 | C | PHE | A | 619 | 6.150 | 6.017 | 16.996 | 1.00 | 9.52 |
| ATOM | 695 | O | PHE | A | 619 | 4.975 | 5.654 | 17.079 | 1.00 | 9.30 |
| ATOM | 696 | N | ASN | A | 620 | 7.001 | 5.490 | 16.119 | 1.00 | 10.15 |
| ATOM | 697 | CA | ASN | A | 620 | 6.583 | 4.428 | 15.204 | 1.00 | 11.34 |
| ATOM | 698 | CB | ASN | A | 620 | 7.694 | 4.123 | 14.194 | 1.00 | 9.10 |
| ATOM | 699 | CG | ASN | A | 620 | 7.658 | 5.052 | 12.999 | 1.00 | 17.16 |
| ATOM | 700 | OD1 | ASN | A | 620 | 8.665 | 5.656 | 12.634 | 1.00 | 19.51 |
| ATOM | 701 | ND2 | ASN | A | 620 | 6.490 | 5.166 | 12.382 | 1.00 | 12.88 |
| ATOM | 702 | C | ASN | A | 620 | 6.206 | 3.161 | 15.956 | 1.00 | 9.23 |
| ATOM | 703 | O | ASN | A | 620 | 5.273 | 2.457 | 15.574 | 1.00 | 10.76 |
| ATOM | 704 | N | THR | A | 621 | 6.936 | 2.871 | 17.026 | 1.00 | 8.56 |
| ATOM | 705 | CA | THR | A | 621 | 6.650 | 1.688 | 17.819 | 1.00 | 9.52 |
| ATOM | 706 | CB | THR | A | 621 | 7.707 | 1.509 | 18.928 | 1.00 | 8.96 |
| ATOM | 707 | OG1 | THR | A | 621 | 9.010 | 1.404 | 18.328 | 1.00 | 9.14 |
| ATOM | 708 | CG2 | THR | A | 621 | 7.428 | 0.244 | 19.736 | 1.00 | 7.93 |
| ATOM | 709 | C | THR | A | 621 | 5.252 | 1.821 | 18.425 | 1.00 | 10.48 |
| ATOM | 710 | O | THR | A | 621 | 4.455 | 0.879 | 18.382 | 1.00 | 10.11 |
| ATOM | 711 | N | ALA | A | 622 | 4.954 | 2.995 | 18.978 | 1.00 | 8.32 |
| ATOM | 712 | CA | ALA | A | 622 | 3.644 | 3.244 | 19.573 | 1.00 | 9.28 |
| ATOM | 713 | CB | ALA | A | 622 | 3.642 | 4.583 | 20.294 | 1.00 | 9.90 |
| ATOM | 714 | C | ALA | A | 622 | 2.567 | 3.224 | 18.488 | 1.00 | 10.02 |
| ATOM | 715 | O | ALA | A | 622 | 1.447 | 2.769 | 18.721 | 1.00 | 8.06 |
| ATOM | 716 | N | GLN | A | 623 | 2.902 | 3.708 | 17.297 | 1.00 | 7.82 |
| ATOM | 717 | CA | GLN | A | 623 | 1.927 | 3.708 | 16.211 | 1.00 | 10.17 |
| ATOM | 718 | CB | GLN | A | 623 | 2.456 | 4.487 | 15.003 | 1.00 | 8.39 |
| ATOM | 719 | CG | GLN | A | 623 | 1.473 | 4.578 | 13.825 | 1.00 | 8.23 |
| ATOM | 720 | CD | GLN | A | 623 | 1.444 | 3.319 | 12.967 | 1.00 | 13.63 |
| ATOM | 721 | OE1 | GLN | A | 623 | 2.484 | 2.730 | 12.682 | 1.00 | 12.75 |
| ATOM | 722 | NE2 | GLN | A | 623 | 0.258 | 2.919 | 12.535 | 1.00 | 11.69 |
| ATOM | 723 | C | GLN | A | 623 | 1.597 | 2.278 | 15.806 | 1.00 | 10.35 |
| ATOM | 724 | O | GLN | A | 623 | 0.449 | 1.962 | 15.501 | 1.00 | 9.61 |
| ATOM | 725 | N | CYS | A | 624 | 2.601 | 1.406 | 15.802 | 1.00 | 9.08 |
| ATOM | 726 | CA | CYS | A | 624 | 2.355 | 0.017 | 15.439 | 1.00 | 9.34 |
| ATOM | 727 | CB | CYS | A | 624 | 3.679 | −0.740 | 15.273 | 1.00 | 11.00 |
| ATOM | 728 | SG | CYS | A | 624 | 3.481 | −2.446 | 14.713 | 1.00 | 11.71 |
| ATOM | 729 | C | CYS | A | 624 | 1.495 | −0.625 | 16.529 | 1.00 | 10.17 |
| ATOM | 730 | O | CYS | A | 624 | 0.631 | −1.448 | 16.239 | 1.00 | 10.64 |
| ATOM | 731 | N | MET | A | 625 | 1.728 | −0.238 | 17.781 | 1.00 | 9.06 |
| ATOM | 732 | CA | MET | A | 625 | 0.933 | −0.764 | 18.892 | 1.00 | 9.38 |
| ATOM | 733 | CB | MET | A | 625 | 1.418 | −0.180 | 20.225 | 1.00 | 8.21 |
| ATOM | 734 | CG | MET | A | 625 | 0.692 | −0.718 | 21.466 | 1.00 | 8.16 |
| ATOM | 735 | SD | MET | A | 625 | 0.904 | −2.512 | 21.728 | 1.00 | 11.88 |
| ATOM | 736 | CE | MET | A | 625 | 2.600 | −2.586 | 22.309 | 1.00 | 11.24 |
| ATOM | 737 | C | MET | A | 625 | −0.529 | −0.376 | 18.661 | 1.00 | 9.31 |
| ATOM | 738 | O | MET | A | 625 | −1.434 | −1.208 | 18.757 | 1.00 | 10.06 |
| ATOM | 739 | N | PHE | A | 626 | −0.757 | 0.896 | 18.353 | 1.00 | 9.47 |
| ATOM | 740 | CA | PHE | A | 626 | −2.112 | 1.377 | 18.092 | 1.00 | 9.60 |
| ATOM | 741 | CB | PHE | A | 626 | −2.077 | 2.859 | 17.725 | 1.00 | 9.70 |
| ATOM | 742 | CG | PHE | A | 626 | −3.432 | 3.451 | 17.461 | 1.00 | 7.25 |
| ATOM | 743 | CD1 | PHE | A | 626 | −4.241 | 3.858 | 18.509 | 1.00 | 13.86 |
| ATOM | 744 | CD2 | PHE | A | 626 | −3.897 | 3.598 | 16.163 | 1.00 | 13.47 |
| ATOM | 745 | CE1 | PHE | A | 626 | −5.491 | 4.411 | 18.267 | 1.00 | 18.88 |
| ATOM | 746 | CE2 | PHE | A | 626 | −5.144 | 4.147 | 15.917 | 1.00 | 11.81 |
| ATOM | 747 | CZ | PHE | A | 626 | −5.942 | 4.552 | 16.970 | 1.00 | 16.53 |
| ATOM | 748 | C | PHE | A | 626 | −2.737 | 0.579 | 16.940 | 1.00 | 10.98 |
| ATOM | 749 | O | PHE | A | 626 | −3.862 | 0.084 | 17.047 | 1.00 | 12.71 |
| ATOM | 750 | N | ALA | A | 627 | −1.999 | 0.455 | 15.838 | 1.00 | 7.24 |
| ATOM | 751 | CA | ALA | A | 627 | −2.486 | −0.279 | 14.675 | 1.00 | 9.31 |
| ATOM | 752 | CB | ALA | A | 627 | −1.454 | −0.220 | 13.549 | 1.00 | 14.42 |
| ATOM | 753 | C | ALA | A | 627 | −2.803 | −1.734 | 15.024 | 1.00 | 11.79 |
| ATOM | 754 | O | ALA | A | 627 | −3.815 | −2.276 | 14.583 | 1.00 | 11.59 |
| ATOM | 755 | N | ALA | A | 628 | −1.945 | −2.361 | 15.824 | 1.00 | 10.63 |
| ATOM | 756 | CA | ALA | A | 628 | −2.158 | −3.749 | 16.219 | 1.00 | 11.00 |
| ATOM | 757 | CB | ALA | A | 628 | −0.934 | −4.280 | 16.961 | 1.00 | 11.43 |
| ATOM | 758 | C | ALA | A | 628 | −3.398 | −3.877 | 17.099 | 1.00 | 11.16 |
| ATOM | 759 | O | ALA | A | 628 | −4.152 | −4.845 | 16.994 | 1.00 | 12.30 |
| ATOM | 760 | N | LEU | A | 629 | −3.608 | −2.896 | 17.970 | 1.00 | 9.63 |
| ATOM | 761 | CA | LEU | A | 629 | −4.765 | −2.924 | 18.856 | 1.00 | 9.96 |
| ATOM | 762 | CB | LEU | A | 629 | −4.627 | −1.858 | 19.945 | 1.00 | 12.79 |
| ATOM | 763 | CG | LEU | A | 629 | −3.538 | −2.115 | 20.990 | 1.00 | 10.87 |
| ATOM | 764 | CD1 | LEU | A | 629 | −3.286 | −0.859 | 21.792 | 1.00 | 11.38 |
| ATOM | 765 | CD2 | LEU | A | 629 | −3.956 | −3.264 | 21.897 | 1.00 | 14.99 |
| ATOM | 766 | C | LEU | A | 629 | −6.051 | −2.692 | 18.073 | 1.00 | 12.31 |
| ATOM | 767 | O | LEU | A | 629 | −7.084 | −3.300 | 18.364 | 1.00 | 11.63 |
| ATOM | 768 | N | LYS | A | 630 | −5.973 | −1.822 | 17.070 | 1.00 | 11.64 |
| ATOM | 769 | CA | LYS | A | 630 | −7.125 | −1.479 | 16.242 | 1.00 | 12.87 |
| ATOM | 770 | CB | LYS | A | 630 | −6.984 | −0.037 | 15.738 | 1.00 | 15.53 |
| ATOM | 771 | CG | LYS | A | 630 | −7.084 | 1.030 | 16.820 | 1.00 | 13.30 |
| ATOM | 772 | CD | LYS | A | 630 | −8.495 | 1.104 | 17.385 | 1.00 | 19.60 |
| ATOM | 773 | CE | LYS | A | 630 | −8.698 | 2.349 | 18.245 | 1.00 | 18.93 |
| ATOM | 774 | NZ | LYS | A | 630 | −7.877 | 2.334 | 19.481 | 1.00 | 40.94 |
| ATOM | 775 | C | LYS | A | 630 | −7.323 | −2.422 | 15.049 | 1.00 | 13.54 |
| ATOM | 776 | O | LYS | A | 630 | −8.122 | −3.350 | 15.107 | 1.00 | 12.81 |
| ATOM | 777 | N | ALA | A | 631 | −6.597 | −2.170 | 13.967 | 1.00 | 13.05 |
| ATOM | 778 | CA | ALA | A | 631 | −6.701 | −2.997 | 12.770 | 1.00 | 14.94 |
| ATOM | 779 | CB | ALA | A | 631 | −5.763 | −2.460 | 11.688 | 1.00 | 17.58 |
| ATOM | 780 | C | ALA | A | 631 | −6.374 | −4.461 | 13.068 | 1.00 | 16.22 |
| ATOM | 781 | O | ALA | A | 631 | −6.960 | −5.375 | 12.479 | 1.00 | 15.44 |
| ATOM | 782 | N | GLY | A | 632 | −5.439 | −4.670 | 13.990 | 1.00 | 12.88 |
| ATOM | 783 | CA | GLY | A | 632 | −5.028 | −6.014 | 14.359 | 1.00 | 13.10 |
| ATOM | 784 | C | GLY | A | 632 | −5.995 | −6.711 | 15.298 | 1.00 | 13.12 |
| ATOM | 785 | O | GLY | A | 632 | −5.850 | −7.902 | 15.576 | 1.00 | 15.03 |
| ATOM | 786 | N | LYS | A | 633 | −6.968 | −5.959 | 15.803 | 1.00 | 13.75 |
| ATOM | 787 | CA | LYS | A | 633 | −7.997 | −6.497 | 16.692 | 1.00 | 14.20 |
| ATOM | 788 | CB | LYS | A | 633 | −8.815 | −7.566 | 15.954 | 1.00 | 13.72 |
| ATOM | 789 | CG | LYS | A | 633 | −9.438 | −7.109 | 14.631 | 1.00 | 23.64 |
| ATOM | 790 | CD | LYS | A | 633 | −10.532 | −6.072 | 14.834 | 1.00 | 30.20 |
| ATOM | 791 | CE | LYS | A | 633 | −11.182 | −5.672 | 13.510 | 1.00 | 34.87 |
| ATOM | 792 | NZ | LYS | A | 633 | −10.227 | −4.989 | 12.594 | 1.00 | 46.21 |
| ATOM | 793 | C | LYS | A | 633 | −7.464 | −7.085 | 17.997 | 1.00 | 14.18 |
| ATOM | 794 | O | LYS | A | 633 | −8.048 | −8.024 | 18.540 | 1.00 | 14.72 |
| ATOM | 795 | N | ILE | A | 634 | −6.366 | −6.536 | 18.507 | 1.00 | 13.07 |
| ATOM | 796 | CA | ILE | A | 634 | −5.801 | −7.037 | 19.755 | 1.00 | 12.44 |
| ATOM | 797 | CB | ILE | A | 634 | −4.262 | −6.819 | 19.799 | 1.00 | 13.52 |
| ATOM | 798 | CG2 | ILE | A | 634 | −3.709 | −7.236 | 21.156 | 1.00 | 14.73 |
| ATOM | 799 | CG1 | ILE | A | 634 | −3.581 | −7.632 | 18.694 | 1.00 | 14.10 |
| ATOM | 800 | CD1 | ILE | A | 634 | −3.757 | −9.128 | 18.829 | 1.00 | 14.44 |
| ATOM | 801 | C | ILE | A | 634 | −6.439 | −6.346 | 20.967 | 1.00 | 12.23 |
| ATOM | 802 | O | ILE | A | 634 | −6.456 | −6.894 | 22.067 | 1.00 | 12.06 |
| ATOM | 803 | N | GLN | A | 635 | −6.990 | −5.155 | 20.752 | 1.00 | 13.17 |
| ATOM | 804 | CA | GLN | A | 635 | −7.597 | −4.377 | 21.834 | 1.00 | 12.59 |
| ATOM | 805 | CB | GLN | A | 635 | −8.330 | −3.158 | 21.267 | 1.00 | 14.77 |
| ATOM | 806 | CG | GLN | A | 635 | −8.796 | −2.180 | 22.345 | 1.00 | 17.07 |
| ATOM | 807 | CD | GLN | A | 635 | −9.479 | −0.949 | 21.778 | 1.00 | 17.87 |
| ATOM | 808 | OE1 | GLN | A | 635 | −9.080 | −0.425 | 20.741 | 1.00 | 15.93 |
| ATOM | 809 | NE2 | GLN | A | 635 | −10.509 | −0.473 | 22.471 | 1.00 | 27.59 |
| ATOM | 810 | C | GLN | A | 635 | −8.549 | −5.127 | 22.762 | 1.00 | 13.46 |
| ATOM | 811 | O | GLN | A | 635 | −8.382 | −5.107 | 23.984 | 1.00 | 12.66 |
| ATOM | 812 | N | ASN | A | 636 | −9.554 | −5.778 | 22.188 | 1.00 | 12.36 |
| ATOM | 813 | CA | ASN | A | 636 | −10.533 | −6.493 | 22.996 | 1.00 | 15.16 |
| ATOM | 814 | CB | ASN | A | 636 | −11.736 | −6.872 | 22.130 | 1.00 | 13.54 |
| ATOM | 815 | CG | ASN | A | 636 | −12.508 | −5.655 | 21.658 | 1.00 | 19.96 |
| ATOM | 816 | OD1 | ASN | A | 636 | −12.211 | −4.526 | 22.059 | 1.00 | 19.43 |
| ATOM | 817 | ND2 | ASN | A | 636 | −13.506 | −5.874 | 20.810 | 1.00 | 20.88 |
| ATOM | 818 | C | ASN | A | 636 | −10.002 | −7.708 | 23.743 | 1.00 | 14.39 |
| ATOM | 819 | O | ASN | A | 636 | −10.720 | −8.312 | 24.539 | 1.00 | 17.61 |
| ATOM | 820 | N | LYS | A | 637 | −8.744 | −8.060 | 23.500 | 1.00 | 13.69 |
| ATOM | 821 | CA | LYS | A | 637 | −8.132 | −9.194 | 24.180 | 1.00 | 13.94 |
| ATOM | 822 | CB | LYS | A | 637 | −7.124 | −9.893 | 23.262 | 1.00 | 13.19 |
| ATOM | 823 | CG | LYS | A | 637 | −7.667 | −10.328 | 21.910 | 1.00 | 16.41 |
| ATOM | 824 | CD | LYS | A | 637 | −6.599 | −11.079 | 21.128 | 1.00 | 18.79 |
| ATOM | 825 | CE | LYS | A | 637 | −7.110 | −11.542 | 19.773 | 1.00 | 21.00 |
| ATOM | 826 | NZ | LYS | A | 637 | −8.266 | −12.467 | 19.893 | 1.00 | 27.72 |
| ATOM | 827 | C | LYS | A | 637 | −7.400 | −8.708 | 25.435 | 1.00 | 14.29 |
| ATOM | 828 | O | LYS | A | 637 | −6.930 | −9.514 | 26.237 | 1.00 | 15.91 |
| ATOM | 829 | N | LEU | A | 638 | −7.316 | −7.391 | 25.611 | 1.00 | 13.88 |
| ATOM | 830 | CA | LEU | A | 638 | −6.602 | −6.828 | 26.757 | 1.00 | 14.02 |
| ATOM | 831 | CB | LEU | A | 638 | −5.426 | −5.988 | 26.256 | 1.00 | 14.42 |
| ATOM | 832 | CG | LEU | A | 638 | −4.496 | −6.595 | 25.201 | 1.00 | 13.65 |
| ATOM | 833 | CD1 | LEU | A | 638 | −3.405 | −5.589 | 24.860 | 1.00 | 13.98 |
| ATOM | 834 | CD2 | LEU | A | 638 | −3.881 | −7.885 | 25.718 | 1.00 | 12.70 |
| ATOM | 835 | C | LEU | A | 638 | −7.464 | −5.973 | 27.687 | 1.00 | 13.83 |
| ATOM | 836 | O | LEU | A | 638 | −8.551 | −5.529 | 27.313 | 1.00 | 16.24 |
| ATOM | 837 | N | THR | A | 639 | −6.968 | −5.745 | 28.902 | 1.00 | 13.04 |
| ATOM | 838 | CA | THR | A | 639 | −7.665 | −4.913 | 29.885 | 1.00 | 12.97 |
| ATOM | 839 | CB | THR | A | 639 | −7.225 | −5.244 | 31.323 | 1.00 | 9.72 |
| ATOM | 840 | OG1 | THR | A | 639 | −5.883 | −4.777 | 31.534 | 1.00 | 11.58 |
| ATOM | 841 | CG2 | THR | A | 639 | −7.284 | −6.752 | 31.574 | 1.00 | 13.01 |
| ATOM | 842 | C | THR | A | 639 | −7.320 | −3.443 | 29.627 | 1.00 | 11.14 |
| ATOM | 843 | O | THR | A | 639 | −6.369 | −3.147 | 28.910 | 1.00 | 12.39 |
| ATOM | 844 | N | ASP | A | 640 | −8.082 | −2.524 | 30.216 | 1.00 | 11.83 |
| ATOM | 845 | CA | ASP | A | 640 | −7.814 | −1.098 | 30.027 | 1.00 | 11.03 |
| ATOM | 846 | CB | ASP | A | 640 | −8.846 | −0.243 | 30.774 | 1.00 | 9.58 |
| ATOM | 847 | CG | ASP | A | 640 | −10.238 | −0.368 | 30.191 | 1.00 | 23.27 |
| ATOM | 848 | OD1 | ASP | A | 640 | −10.357 | −0.828 | 29.037 | 1.00 | 21.74 |
| ATOM | 849 | OD2 | ASP | A | 640 | −11.211 | 0.011 | 30.879 | 1.00 | 18.96 |
| ATOM | 850 | C | ASP | A | 640 | −6.419 | −0.724 | 30.519 | 1.00 | 10.85 |
| ATOM | 851 | O | ASP | A | 640 | −5.708 | 0.058 | 29.881 | 1.00 | 11.05 |
| ATOM | 852 | N | LEU | A | 641 | −6.031 | −1.290 | 31.655 | 1.00 | 11.16 |
| ATOM | 853 | CA | LEU | A | 641 | −4.727 | −1.004 | 32.235 | 1.00 | 10.51 |
| ATOM | 854 | CB | LEU | A | 641 | −4.628 | −1.606 | 33.640 | 1.00 | 13.05 |
| ATOM | 855 | CG | LEU | A | 641 | −5.633 | −1.081 | 34.674 | 1.00 | 13.28 |
| ATOM | 856 | CD1 | LEU | A | 641 | −5.383 | −1.762 | 36.005 | 1.00 | 14.91 |
| ATOM | 857 | CD2 | LEU | A | 641 | −5.506 | 0.432 | 34.813 | 1.00 | 12.98 |
| ATOM | 858 | C | LEU | A | 641 | −3.593 | −1.527 | 31.359 | 1.00 | 10.02 |
| ATOM | 859 | O | LEU | A | 641 | −2.550 | −0.883 | 31.237 | 1.00 | 9.79 |
| ATOM | 860 | N | GLU | A | 642 | −3.791 | −2.692 | 30.748 | 1.00 | 9.49 |
| ATOM | 861 | CA | GLU | A | 642 | −2.756 | −3.253 | 29.886 | 1.00 | 9.55 |
| ATOM | 862 | CB | GLU | A | 642 | −3.120 | −4.687 | 29.493 | 1.00 | 13.24 |
| ATOM | 863 | CG | GLU | A | 642 | −3.215 | −5.605 | 30.705 | 1.00 | 17.56 |
| ATOM | 864 | CD | GLU | A | 642 | −3.645 | −7.021 | 30.373 | 1.00 | 14.47 |
| ATOM | 865 | OE1 | GLU | A | 642 | −4.411 | −7.212 | 29.404 | 1.00 | 12.63 |
| ATOM | 866 | OE2 | GLU | A | 642 | −3.233 | −7.949 | 31.108 | 1.00 | 15.36 |
| ATOM | 867 | C | GLU | A | 642 | −2.568 | −2.366 | 28.656 | 1.00 | 9.56 |
| ATOM | 868 | O | GLU | A | 642 | −1.442 | −2.135 | 28.217 | 1.00 | 10.36 |
| ATOM | 869 | N | ILE | A | 643 | −3.665 | −1.847 | 28.112 | 1.00 | 9.98 |
| ATOM | 870 | CA | ILE | A | 643 | −3.585 | −0.978 | 26.945 | 1.00 | 11.53 |
| ATOM | 871 | CB | ILE | A | 643 | −4.992 | −0.683 | 26.391 | 1.00 | 12.52 |
| ATOM | 872 | CG2 | ILE | A | 643 | −4.915 | 0.367 | 25.281 | 1.00 | 11.83 |
| ATOM | 873 | CG1 | ILE | A | 643 | −5.614 | −1.984 | 25.885 | 1.00 | 11.82 |
| ATOM | 874 | CD1 | ILE | A | 643 | −7.089 | −1.872 | 25.553 | 1.00 | 18.49 |
| ATOM | 875 | C | ILE | A | 643 | −2.883 | 0.323 | 27.339 | 1.00 | 9.56 |
| ATOM | 876 | O | ILE | A | 643 | −1.993 | 0.806 | 26.635 | 1.00 | 10.76 |
| ATOM | 877 | N | LEU | A | 644 | −3.278 | 0.880 | 28.479 | 1.00 | 9.68 |
| ATOM | 878 | CA | LEU | A | 644 | −2.663 | 2.105 | 28.974 | 1.00 | 9.78 |
| ATOM | 879 | CB | LEU | A | 644 | −3.285 | 2.482 | 30.321 | 1.00 | 11.15 |
| ATOM | 880 | CG | LEU | A | 644 | −2.658 | 3.643 | 31.093 | 1.00 | 14.02 |
| ATOM | 881 | CD1 | LEU | A | 644 | −2.853 | 4.946 | 30.328 | 1.00 | 14.15 |
| ATOM | 882 | CD2 | LEU | A | 644 | −3.315 | 3.735 | 32.471 | 1.00 | 14.10 |
| ATOM | 883 | C | LEU | A | 644 | −1.152 | 1.899 | 29.139 | 1.00 | 9.98 |
| ATOM | 884 | O | LEU | A | 644 | −0.342 | 2.720 | 28.694 | 1.00 | 9.97 |
| ATOM | 885 | N | ALA | A | 645 | −0.778 | 0.788 | 29.766 | 1.00 | 8.98 |
| ATOM | 886 | CA | ALA | A | 645 | 0.634 | 0.492 | 30.008 | 1.00 | 8.84 |
| ATOM | 887 | CB | ALA | A | 645 | 0.763 | −0.724 | 30.928 | 1.00 | 8.17 |
| ATOM | 888 | C | ALA | A | 645 | 1.414 | 0.256 | 28.718 | 1.00 | 9.39 |
| ATOM | 889 | O | ALA | A | 645 | 2.556 | 0.687 | 28.598 | 1.00 | 9.92 |
| ATOM | 890 | N | LEU | A | 646 | 0.799 | −0.430 | 27.761 | 1.00 | 8.76 |
| ATOM | 891 | CA | LEU | A | 646 | 1.462 | −0.708 | 26.488 | 1.00 | 8.34 |
| ATOM | 892 | CB | LEU | A | 646 | 0.607 | −1.652 | 25.644 | 1.00 | 10.30 |
| ATOM | 893 | CG | LEU | A | 646 | 0.541 | −3.101 | 26.128 | 1.00 | 11.47 |
| ATOM | 894 | CD1 | LEU | A | 646 | −0.557 | −3.832 | 25.379 | 1.00 | 15.28 |
| ATOM | 895 | CD2 | LEU | A | 646 | 1.879 | −3.779 | 25.906 | 1.00 | 10.48 |
| ATOM | 896 | C | LEU | A | 646 | 1.761 | 0.550 | 25.682 | 1.00 | 7.55 |
| ATOM | 897 | O | LEU | A | 646 | 2.837 | 0.676 | 25.094 | 1.00 | 9.05 |
| ATOM | 898 | N | LEU | A | 647 | 0.812 | 1.479 | 25.643 | 1.00 | 7.57 |
| ATOM | 899 | CA | LEU | A | 647 | 1.020 | 2.713 | 24.889 | 1.00 | 8.87 |
| ATOM | 900 | CB | LEU | A | 647 | −0.283 | 3.516 | 24.803 | 1.00 | 7.82 |
| ATOM | 901 | CG | LEU | A | 647 | −0.214 | 4.722 | 23.862 | 1.00 | 10.06 |
| ATOM | 902 | CD1 | LEU | A | 647 | 0.194 | 4.251 | 22.470 | 1.00 | 13.81 |
| ATOM | 903 | CD2 | LEU | A | 647 | −1.560 | 5.437 | 23.820 | 1.00 | 12.05 |
| ATOM | 904 | C | LEU | A | 647 | 2.120 | 3.550 | 25.540 | 1.00 | 8.12 |
| ATOM | 905 | O | LEU | A | 647 | 3.011 | 4.070 | 24.863 | 1.00 | 8.78 |
| ATOM | 906 | N | ILE | A | 648 | 2.056 | 3.676 | 26.860 | 1.00 | 9.66 |
| ATOM | 907 | CA | ILE | A | 648 | 3.065 | 4.429 | 27.597 | 1.00 | 8.86 |
| ATOM | 908 | CB | ILE | A | 648 | 2.692 | 4.501 | 29.100 | 1.00 | 10.81 |
| ATOM | 909 | CG2 | ILE | A | 648 | 3.851 | 5.063 | 29.917 | 1.00 | 7.70 |
| ATOM | 910 | CG1 | ILE | A | 648 | 1.439 | 5.364 | 29.266 | 1.00 | 11.45 |
| ATOM | 911 | CD1 | ILE | A | 648 | 0.921 | 5.437 | 30.688 | 1.00 | 9.57 |
| ATOM | 912 | C | ILE | A | 648 | 4.427 | 3.756 | 27.424 | 1.00 | 8.38 |
| ATOM | 913 | O | ILE | A | 648 | 5.439 | 4.427 | 27.180 | 1.00 | 9.63 |
| ATOM | 914 | N | ALA | A | 649 | 4.456 | 2.430 | 27.536 | 1.00 | 9.32 |
| ATOM | 915 | CA | ALA | A | 649 | 5.710 | 1.697 | 27.388 | 1.00 | 9.98 |
| ATOM | 916 | CB | ALA | A | 649 | 5.506 | 0.214 | 27.703 | 1.00 | 11.64 |
| ATOM | 917 | C | ALA | A | 649 | 6.285 | 1.853 | 25.989 | 1.00 | 9.64 |
| ATOM | 918 | O | ALA | A | 649 | 7.471 | 2.126 | 25.831 | 1.00 | 10.34 |
| ATOM | 919 | N | ALA | A | 650 | 5.449 | 1.680 | 24.971 | 1.00 | 11.02 |
| ATOM | 920 | CA | ALA | A | 650 | 5.914 | 1.814 | 23.591 | 1.00 | 9.43 |
| ATOM | 921 | CB | ALA | A | 650 | 4.747 | 1.618 | 22.617 | 1.00 | 8.79 |
| ATOM | 922 | C | ALA | A | 650 | 6.561 | 3.183 | 23.359 | 1.00 | 8.11 |
| ATOM | 923 | O | ALA | A | 650 | 7.628 | 3.292 | 22.748 | 1.00 | 9.60 |
| ATOM | 924 | N | LEU | A | 651 | 5.914 | 4.233 | 23.851 | 1.00 | 9.58 |
| ATOM | 925 | CA | LEU | A | 651 | 6.443 | 5.582 | 23.679 | 1.00 | 10.27 |
| ATOM | 926 | CB | LEU | A | 651 | 5.383 | 6.613 | 24.066 | 1.00 | 9.84 |
| ATOM | 927 | CG | LEU | A | 651 | 4.198 | 6.717 | 23.101 | 1.00 | 6.24 |
| ATOM | 928 | CD1 | LEU | A | 651 | 3.038 | 7.432 | 23.772 | 1.00 | 14.57 |
| ATOM | 929 | CD2 | LEU | A | 651 | 4.637 | 7.463 | 21.846 | 1.00 | 11.43 |
| ATOM | 930 | C | LEU | A | 651 | 7.706 | 5.848 | 24.488 | 1.00 | 10.46 |
| ATOM | 931 | O | LEU | A | 651 | 8.573 | 6.611 | 24.066 | 1.00 | 9.75 |
| ATOM | 932 | N | SER | A | 652 | 7.807 | 5.194 | 25.640 | 1.00 | 9.58 |
| ATOM | 933 | CA | SER | A | 652 | 8.935 | 5.394 | 26.542 | 1.00 | 10.30 |
| ATOM | 934 | CB | SER | A | 652 | 8.422 | 5.453 | 27.983 | 1.00 | 9.30 |
| ATOM | 935 | OG | SER | A | 652 | 7.367 | 6.379 | 28.133 | 1.00 | 11.78 |
| ATOM | 936 | C | SER | A | 652 | 10.026 | 4.334 | 26.504 | 1.00 | 8.64 |
| ATOM | 937 | O | SER | A | 652 | 11.068 | 4.520 | 27.125 | 1.00 | 9.90 |
| ATOM | 938 | N | HIS | A | 653 | 9.805 | 3.249 | 25.765 | 1.00 | 9.64 |
| ATOM | 939 | CA | HIS | A | 653 | 10.739 | 2.128 | 25.769 | 1.00 | 9.99 |
| ATOM | 940 | CB | HIS | A | 653 | 10.164 | 0.963 | 24.952 | 1.00 | 8.26 |
| ATOM | 941 | CG | HIS | A | 653 | 10.546 | 0.980 | 23.510 | 1.00 | 8.55 |
| ATOM | 942 | CD2 | HIS | A | 653 | 11.529 | 0.333 | 22.842 | 1.00 | 9.53 |
| ATOM | 943 | ND1 | HIS | A | 653 | 9.881 | 1.740 | 22.569 | 1.00 | 10.01 |
| ATOM | 944 | CE1 | HIS | A | 653 | 10.440 | 1.559 | 21.387 | 1.00 | 6.46 |
| ATOM | 945 | NE2 | HIS | A | 653 | 11.444 | 0.708 | 21.526 | 1.00 | 6.65 |
| ATOM | 946 | C | HIS | A | 653 | 12.210 | 2.321 | 25.412 | 1.00 | 10.62 |
| ATOM | 947 | O | HIS | A | 653 | 13.024 | 1.459 | 25.741 | 1.00 | 8.86 |
| ATOM | 948 | N | ASP | A | 654 | 12.559 | 3.424 | 24.752 | 1.00 | 9.82 |
| ATOM | 949 | CA | ASP | A | 654 | 13.959 | 3.673 | 24.406 | 1.00 | 9.46 |
| ATOM | 950 | CB | ASP | A | 654 | 14.184 | 3.580 | 22.889 | 1.00 | 10.10 |
| ATOM | 951 | CG | ASP | A | 654 | 14.509 | 2.172 | 22.432 | 1.00 | 10.77 |
| ATOM | 952 | OD1 | ASP | A | 654 | 15.159 | 1.444 | 23.204 | 1.00 | 9.55 |
| ATOM | 953 | OD2 | ASP | A | 654 | 14.141 | 1.812 | 21.294 | 1.00 | 8.85 |
| ATOM | 954 | C | ASP | A | 654 | 14.477 | 5.024 | 24.896 | 1.00 | 9.92 |
| ATOM | 955 | O | ASP | A | 654 | 15.500 | 5.507 | 24.413 | 1.00 | 9.67 |
| ATOM | 956 | N | LEU | A | 655 | 13.783 | 5.619 | 25.865 | 1.00 | 9.19 |
| ATOM | 957 | CA | LEU | A | 655 | 14.169 | 6.922 | 26.408 | 1.00 | 10.06 |
| ATOM | 958 | CB | LEU | A | 655 | 13.364 | 7.214 | 27.677 | 1.00 | 8.55 |
| ATOM | 959 | CG | LEU | A | 655 | 11.900 | 7.603 | 27.479 | 1.00 | 10.24 |
| ATOM | 960 | CD1 | LEU | A | 655 | 11.151 | 7.468 | 28.805 | 1.00 | 9.10 |
| ATOM | 961 | CD2 | LEU | A | 655 | 11.819 | 9.028 | 26.944 | 1.00 | 15.71 |
| ATOM | 962 | C | LEU | A | 655 | 15.658 | 7.101 | 26.713 | 1.00 | 10.50 |
| ATOM | 963 | O | LEU | A | 655 | 16.279 | 6.268 | 27.372 | 1.00 | 9.92 |
| ATOM | 964 | N | ASP | A | 656 | 16.215 | 8.209 | 26.232 | 1.00 | 8.66 |
| ATOM | 965 | CA | ASP | A | 656 | 17.622 | 8.542 | 26.448 | 1.00 | 8.66 |
| ATOM | 966 | CB | ASP | A | 656 | 17.867 | 8.811 | 27.941 | 1.00 | 11.08 |
| ATOM | 967 | CG | ASP | A | 656 | 19.211 | 9.475 | 28.207 | 1.00 | 16.07 |
| ATOM | 968 | OD1 | ASP | A | 656 | 19.576 | 10.406 | 27.460 | 1.00 | 11.04 |
| ATOM | 969 | OD2 | ASP | A | 656 | 19.897 | 9.074 | 29.173 | 1.00 | 13.88 |
| ATOM | 970 | C | ASP | A | 656 | 18.600 | 7.483 | 25.939 | 1.00 | 8.59 |
| ATOM | 971 | O | ASP | A | 656 | 19.670 | 7.288 | 26.511 | 1.00 | 9.89 |
| ATOM | 972 | N | HIS | A | 657 | 18.242 | 6.806 | 24.851 | 1.00 | 9.07 |
| ATOM | 973 | CA | HIS | A | 657 | 19.118 | 5.790 | 24.284 | 1.00 | 10.63 |
| ATOM | 974 | CB | HIS | A | 657 | 18.412 | 5.068 | 23.137 | 1.00 | 9.06 |
| ATOM | 975 | CG | HIS | A | 657 | 19.095 | 3.810 | 22.712 | 1.00 | 8.47 |
| ATOM | 976 | CD2 | HIS | A | 657 | 20.347 | 3.589 | 22.245 | 1.00 | 7.97 |
| ATOM | 977 | ND1 | HIS | A | 657 | 18.480 | 2.575 | 22.748 | 1.00 | 12.46 |
| ATOM | 978 | CE1 | HIS | A | 657 | 19.322 | 1.652 | 22.322 | 1.00 | 5.38 |
| ATOM | 979 | NE2 | HIS | A | 657 | 20.464 | 2.242 | 22.011 | 1.00 | 12.33 |
| ATOM | 980 | C | HIS | A | 657 | 20.404 | 6.453 | 23.778 | 1.00 | 11.40 |
| ATOM | 981 | O | HIS | A | 657 | 20.362 | 7.404 | 22.993 | 1.00 | 11.45 |
| ATOM | 982 | N | PRO | A | 658 | 21.567 | 5.957 | 24.230 | 1.00 | 11.01 |
| ATOM | 983 | CD | PRO | A | 658 | 21.697 | 4.963 | 25.311 | 1.00 | 10.94 |
| ATOM | 984 | CA | PRO | A | 658 | 22.884 | 6.479 | 23.850 | 1.00 | 10.19 |
| ATOM | 985 | CB | PRO | A | 658 | 23.792 | 5.930 | 24.944 | 1.00 | 9.66 |
| ATOM | 986 | CG | PRO | A | 658 | 23.171 | 4.618 | 25.252 | 1.00 | 16.61 |
| ATOM | 987 | C | PRO | A | 658 | 23.386 | 6.123 | 22.451 | 1.00 | 11.02 |
| ATOM | 988 | O | PRO | A | 658 | 24.428 | 6.627 | 22.021 | 1.00 | 12.41 |
| ATOM | 989 | N | GLY | A | 659 | 22.659 | 5.265 | 21.744 | 1.00 | 8.82 |
| ATOM | 990 | CA | GLY | A | 659 | 23.080 | 4.888 | 20.405 | 1.00 | 8.78 |
| ATOM | 991 | C | GLY | A | 659 | 24.115 | 3.781 | 20.356 | 1.00 | 9.97 |
| ATOM | 992 | O | GLY | A | 659 | 24.750 | 3.566 | 19.316 | 1.00 | 12.32 |
| ATOM | 993 | N | VAL | A | 660 | 24.305 | 3.095 | 21.480 | 1.00 | 8.93 |
| ATOM | 994 | CA | VAL | A | 660 | 25.248 | 1.983 | 21.564 | 1.00 | 9.69 |
| ATOM | 995 | CB | VAL | A | 660 | 26.554 | 2.387 | 22.301 | 1.00 | 10.19 |
| ATOM | 996 | CG1 | VAL | A | 660 | 27.358 | 3.355 | 21.434 | 1.00 | 12.07 |
| ATOM | 997 | CG2 | VAL | A | 660 | 26.236 | 3.020 | 23.642 | 1.00 | 12.93 |
| ATOM | 998 | C | VAL | A | 660 | 24.545 | 0.834 | 22.289 | 1.00 | 10.76 |
| ATOM | 999 | O | VAL | A | 660 | 23.601 | 1.059 | 23.052 | 1.00 | 10.23 |
| ATOM | 1000 | N | SER | A | 661 | 25.002 | −0.392 | 22.049 | 1.00 | 7.63 |
| ATOM | 1001 | CA | SER | A | 661 | 24.371 | −1.577 | 22.624 | 1.00 | 10.33 |
| ATOM | 1002 | CB | SER | A | 661 | 24.748 | −2.801 | 21.793 | 1.00 | 8.76 |
| ATOM | 1003 | OG | SER | A | 661 | 26.099 | −3.151 | 22.021 | 1.00 | 9.56 |
| ATOM | 1004 | C | SER | A | 661 | 24.687 | −1.864 | 24.088 | 1.00 | 9.36 |
| ATOM | 1005 | O | SER | A | 661 | 25.549 | −1.230 | 24.696 | 1.00 | 10.42 |
| ATOM | 1006 | N | ASN | A | 662 | 23.969 | −2.838 | 24.643 | 1.00 | 10.10 |
| ATOM | 1007 | CA | ASN | A | 662 | 24.165 | −3.261 | 26.026 | 1.00 | 8.94 |
| ATOM | 1008 | CB | ASN | A | 662 | 23.177 | −4.369 | 26.384 | 1.00 | 12.24 |
| ATOM | 1009 | CG | ASN | A | 662 | 21.832 | −3.836 | 26.824 | 1.00 | 11.27 |
| ATOM | 1010 | OD1 | ASN | A | 662 | 20.794 | −4.457 | 26.592 | 1.00 | 19.20 |
| ATOM | 1011 | ND2 | ASN | A | 662 | 21.844 | −2.689 | 27.483 | 1.00 | 5.90 |
| ATOM | 1012 | C | ASN | A | 662 | 25.586 | −3.781 | 26.161 | 1.00 | 11.89 |
| ATOM | 1013 | O | ASN | A | 662 | 26.289 | −3.459 | 27.116 | 1.00 | 12.39 |
| ATOM | 1014 | N | GLN | A | 663 | 26.010 | −4.598 | 25.199 | 1.00 | 10.34 |
| ATOM | 1015 | CA | GLN | A | 663 | 27.356 | −5.139 | 25.246 | 1.00 | 11.06 |
| ATOM | 1016 | CB | GLN | A | 663 | 27.578 | −6.120 | 24.083 | 1.00 | 14.83 |
| ATOM | 1017 | CG | GLN | A | 663 | 26.677 | −7.373 | 24.197 | 1.00 | 25.28 |
| ATOM | 1018 | CD | GLN | A | 663 | 25.283 | −7.191 | 23.575 | 1.00 | 24.96 |
| ATOM | 1019 | OE1 | GLN | A | 663 | 24.919 | −6.113 | 23.106 | 1.00 | 31.10 |
| ATOM | 1020 | NE2 | GLN | A | 663 | 24.497 | −8.271 | 23.572 | 1.00 | 46.85 |
| ATOM | 1021 | C | GLN | A | 663 | 28.422 | −4.039 | 25.236 | 1.00 | 13.51 |
| ATOM | 1022 | O | GLN | A | 663 | 29.439 | −4.161 | 25.921 | 1.00 | 12.70 |
| ATOM | 1023 | N | PHE | A | 664 | 28.184 | −2.956 | 24.504 | 1.00 | 12.64 |
| ATOM | 1024 | CA | PHE | A | 664 | 29.145 | −1.852 | 24.465 | 1.00 | 11.20 |
| ATOM | 1025 | CB | PHE | A | 664 | 28.754 | −0.838 | 23.380 | 1.00 | 12.60 |
| ATOM | 1026 | CG | PHE | A | 664 | 29.669 | 0.355 | 23.296 | 1.00 | 15.21 |
| ATOM | 1027 | CD1 | PHE | A | 664 | 29.554 | 1.407 | 24.198 | 1.00 | 13.93 |
| ATOM | 1028 | CD2 | PHE | A | 664 | 30.668 | 0.409 | 22.336 | 1.00 | 10.34 |
| ATOM | 1029 | CE1 | PHE | A | 664 | 30.419 | 2.484 | 24.144 | 1.00 | 11.59 |
| ATOM | 1030 | CE2 | PHE | A | 664 | 31.541 | 1.487 | 22.275 | 1.00 | 16.42 |
| ATOM | 1031 | CZ | PHE | A | 664 | 31.418 | 2.527 | 23.182 | 1.00 | 18.27 |
| ATOM | 1032 | C | PHE | A | 664 | 29.207 | −1.171 | 25.830 | 1.00 | 11.75 |
| ATOM | 1033 | O | PHE | A | 664 | 30.290 | −0.818 | 26.306 | 1.00 | 11.28 |
| ATOM | 1034 | N | LEU | A | 665 | 28.046 | −0.980 | 26.452 | 1.00 | 11.17 |
| ATOM | 1035 | CA | LEU | A | 665 | 27.982 | −0.346 | 27.768 | 1.00 | 11.46 |
| ATOM | 1036 | CB | LEU | A | 665 | 26.524 | −0.163 | 28.204 | 1.00 | 11.38 |
| ATOM | 1037 | CG | LEU | A | 665 | 25.703 | 0.864 | 27.420 | 1.00 | 9.72 |
| ATOM | 1038 | CD1 | LEU | A | 665 | 24.304 | 0.959 | 28.015 | 1.00 | 16.35 |
| ATOM | 1039 | CD2 | LEU | A | 665 | 26.399 | 2.215 | 27.470 | 1.00 | 13.26 |
| ATOM | 1040 | C | LEU | A | 665 | 28.726 | −1.200 | 28.793 | 1.00 | 13.75 |
| ATOM | 1041 | O | LEU | A | 665 | 29.404 | −0.686 | 29.681 | 1.00 | 11.85 |
| ATOM | 1042 | N | ILE | A | 666 | 28.591 | −2.512 | 28.661 | 1.00 | 12.41 |
| ATOM | 1043 | CA | ILE | A | 666 | 29.259 | −3.444 | 29.559 | 1.00 | 13.52 |
| ATOM | 1044 | CB | ILE | A | 666 | 28.773 | −4.887 | 29.300 | 1.00 | 13.10 |
| ATOM | 1045 | CG2 | ILE | A | 666 | 29.667 | −5.882 | 30.020 | 1.00 | 13.00 |
| ATOM | 1046 | CG1 | ILE | A | 666 | 27.322 | −5.036 | 29.761 | 1.00 | 10.73 |
| ATOM | 1047 | CD1 | ILE | A | 666 | 26.687 | −6.346 | 29.369 | 1.00 | 14.67 |
| ATOM | 1048 | C | ILE | A | 666 | 30.770 | −3.381 | 29.356 | 1.00 | 11.71 |
| ATOM | 1049 | O | ILE | A | 666 | 31.541 | −3.297 | 30.317 | 1.00 | 14.40 |
| ATOM | 1050 | N | ASN | A | 667 | 31.183 | −3.408 | 28.094 | 1.00 | 12.52 |
| ATOM | 1051 | CA | ASN | A | 667 | 32.599 | −3.382 | 27.733 | 1.00 | 11.69 |
| ATOM | 1052 | CB | ASN | A | 667 | 32.745 | −3.652 | 26.232 | 1.00 | 15.94 |
| ATOM | 1053 | CG | ASN | A | 667 | 32.340 | −5.065 | 25.848 | 1.00 | 13.91 |
| ATOM | 1054 | OD1 | ASN | A | 667 | 32.138 | −5.363 | 24.669 | 1.00 | 17.63 |
| ATOM | 1055 | ND2 | ASN | A | 667 | 32.233 | −5.946 | 26.837 | 1.00 | 11.25 |
| ATOM | 1056 | C | ASN | A | 667 | 33.323 | −2.090 | 28.091 | 1.00 | 15.59 |
| ATOM | 1057 | O | ASN | A | 667 | 34.530 | −2.104 | 28.355 | 1.00 | 16.72 |
| ATOM | 1058 | N | THR | A | 668 | 32.601 | −0.974 | 28.098 | 1.00 | 15.05 |
| ATOM | 1059 | CA | THR | A | 668 | 33.211 | 0.308 | 28.428 | 1.00 | 16.29 |
| ATOM | 1060 | CB | THR | A | 668 | 32.670 | 1.432 | 27.520 | 1.00 | 16.12 |
| ATOM | 1061 | OG1 | THR | A | 668 | 31.237 | 1.448 | 27.567 | 1.00 | 19.49 |
| ATOM | 1062 | CG2 | THR | A | 668 | 33.133 | 1.207 | 26.078 | 1.00 | 15.93 |
| ATOM | 1063 | C | THR | A | 668 | 32.995 | 0.672 | 29.895 | 1.00 | 18.10 |
| ATOM | 1064 | O | THR | A | 668 | 33.158 | 1.830 | 30.297 | 1.00 | 20.65 |
| ATOM | 1065 | N | ASN | A | 669 | 32.617 | −0.330 | 30.682 | 1.00 | 17.75 |
| ATOM | 1066 | CA | ASN | A | 669 | 32.402 | −0.177 | 32.115 | 1.00 | 20.15 |
| ATOM | 1067 | CB | ASN | A | 669 | 33.749 | 0.053 | 32.797 | 1.00 | 28.92 |
| ATOM | 1068 | CG | ASN | A | 669 | 34.791 | −0.955 | 32.361 | 1.00 | 34.79 |
| ATOM | 1069 | OD1 | ASN | A | 669 | 34.619 | −2.160 | 32.543 | 1.00 | 40.75 |
| ATOM | 1070 | ND2 | ASN | A | 669 | 35.877 | −0.468 | 31.772 | 1.00 | 36.76 |
| ATOM | 1071 | C | ASN | A | 669 | 31.428 | 0.926 | 32.515 | 1.00 | 20.87 |
| ATOM | 1072 | O | ASN | A | 669 | 31.690 | 1.696 | 33.443 | 1.00 | 21.59 |
| ATOM | 1073 | N | SER | A | 670 | 30.302 | 0.996 | 31.819 | 1.00 | 17.34 |
| ATOM | 1074 | CA | SER | A | 670 | 29.286 | 1.992 | 32.120 | 1.00 | 19.60 |
| ATOM | 1075 | CB | SER | A | 670 | 28.103 | 1.836 | 31.160 | 1.00 | 18.96 |
| ATOM | 1076 | OG | SER | A | 670 | 27.023 | 2.672 | 31.535 | 1.00 | 22.69 |
| ATOM | 1077 | C | SER | A | 670 | 28.803 | 1.787 | 33.551 | 1.00 | 16.24 |
| ATOM | 1078 | O | SER | A | 670 | 28.755 | 0.655 | 34.041 | 1.00 | 14.46 |
| ATOM | 1079 | N | GLU | A | 671 | 28.455 | 2.879 | 34.227 | 1.00 | 18.45 |
| ATOM | 1080 | CA | GLU | A | 671 | 27.951 | 2.775 | 35.589 | 1.00 | 19.69 |
| ATOM | 1081 | CB | GLU | A | 671 | 27.756 | 4.154 | 36.209 | 1.00 | 17.48 |
| ATOM | 1082 | CG | GLU | A | 671 | 28.985 | 5.024 | 36.189 | 1.00 | 31.14 |
| ATOM | 1083 | CD | GLU | A | 671 | 28.901 | 6.156 | 37.191 | 1.00 | 32.78 |
| ATOM | 1084 | OE1 | GLU | A | 671 | 27.830 | 6.796 | 37.291 | 1.00 | 31.62 |
| ATOM | 1085 | OE2 | GLU | A | 671 | 29.914 | 6.406 | 37.875 | 1.00 | 26.52 |
| ATOM | 1086 | C | GLU | A | 671 | 26.611 | 2.058 | 35.549 | 1.00 | 18.77 |
| ATOM | 1087 | O | GLU | A | 671 | 26.253 | 1.332 | 36.482 | 1.00 | 19.75 |
| ATOM | 1088 | N | LEU | A | 672 | 25.868 | 2.281 | 34.466 | 1.00 | 17.92 |
| ATOM | 1089 | CA | LEU | A | 672 | 24.569 | 1.646 | 34.279 | 1.00 | 15.76 |
| ATOM | 1090 | CB | LEU | A | 672 | 23.954 | 2.067 | 32.942 | 1.00 | 12.50 |
| ATOM | 1091 | CG | LEU | A | 672 | 23.478 | 3.509 | 32.771 | 1.00 | 18.92 |
| ATOM | 1092 | CD1 | LEU | A | 672 | 23.101 | 3.741 | 31.314 | 1.00 | 18.65 |
| ATOM | 1093 | CD2 | LEU | A | 672 | 22.292 | 3.766 | 33.678 | 1.00 | 24.52 |
| ATOM | 1094 | C | LEU | A | 672 | 24.738 | 0.130 | 34.291 | 1.00 | 13.50 |
| ATOM | 1095 | O | LEU | A | 672 | 23.961 | −0.586 | 34.912 | 1.00 | 12.92 |
| ATOM | 1096 | N | ALA | A | 673 | 25.755 | −0.358 | 33.590 | 1.00 | 13.11 |
| ATOM | 1097 | CA | ALA | A | 673 | 26.001 | −1.792 | 33.546 | 1.00 | 13.53 |
| ATOM | 1098 | CB | ALA | A | 673 | 27.169 | −2.099 | 32.594 | 1.00 | 11.10 |
| ATOM | 1099 | C | ALA | A | 673 | 26.318 | −2.283 | 34.956 | 1.00 | 13.78 |
| ATOM | 1100 | O | ALA | A | 673 | 25.925 | −3.380 | 35.354 | 1.00 | 12.36 |
| ATOM | 1101 | N | LEU | A | 674 | 27.028 | −1.458 | 35.718 | 1.00 | 15.22 |
| ATOM | 1102 | CA | LEU | A | 674 | 27.394 | −1.829 | 37.076 | 1.00 | 16.65 |
| ATOM | 1103 | CB | LEU | A | 674 | 28.414 | −0.832 | 37.632 | 1.00 | 18.96 |
| ATOM | 1104 | CG | LEU | A | 674 | 29.225 | −1.323 | 38.832 | 1.00 | 26.52 |
| ATOM | 1105 | CD1 | LEU | A | 674 | 29.939 | −2.620 | 38.460 | 1.00 | 24.45 |
| ATOM | 1106 | CD2 | LEU | A | 674 | 30.231 | −0.258 | 39.246 | 1.00 | 20.57 |
| ATOM | 1107 | C | LEU | A | 674 | 26.152 | −1.866 | 37.960 | 1.00 | 15.94 |
| ATOM | 1108 | O | LEU | A | 674 | 25.930 | −2.818 | 38.709 | 1.00 | 16.86 |
| ATOM | 1109 | N | MET | A | 675 | 25.335 | −0.822 | 37.851 | 1.00 | 19.35 |
| ATOM | 1110 | CA | MET | A | 675 | 24.107 | −0.694 | 38.627 | 1.00 | 18.57 |
| ATOM | 1111 | CB | MET | A | 675 | 23.413 | 0.641 | 38.312 | 1.00 | 29.02 |
| ATOM | 1112 | CG | MET | A | 675 | 24.180 | 1.882 | 38.753 | 1.00 | 32.86 |
| ATOM | 1113 | SD | MET | A | 675 | 23.766 | 3.373 | 37.785 | 1.00 | 38.23 |
| ATOM | 1114 | CE | MET | A | 675 | 22.129 | 3.769 | 38.381 | 1.00 | 40.96 |
| ATOM | 1115 | C | MET | A | 675 | 23.133 | −1.830 | 38.346 | 1.00 | 17.83 |
| ATOM | 1116 | O | MET | A | 675 | 22.529 | −2.383 | 39.265 | 1.00 | 17.84 |
| ATOM | 1117 | N | TYR | A | 676 | 23.000 | −2.184 | 37.073 | 1.00 | 15.75 |
| ATOM | 1118 | CA | TYR | A | 676 | 22.052 | −3.212 | 36.679 | 1.00 | 16.85 |
| ATOM | 1119 | CB | TYR | A | 676 | 21.293 | −2.720 | 35.442 | 1.00 | 16.19 |
| ATOM | 1120 | CG | TYR | A | 676 | 20.619 | −1.374 | 35.668 | 1.00 | 20.30 |
| ATOM | 1121 | CD1 | TYR | A | 676 | 19.694 | −1.197 | 36.691 | 1.00 | 31.51 |
| ATOM | 1122 | CE1 | TYR | A | 676 | 19.107 | 0.045 | 36.922 | 1.00 | 18.89 |
| ATOM | 1123 | CD2 | TYR | A | 676 | 20.934 | −0.276 | 34.877 | 1.00 | 23.78 |
| ATOM | 1124 | CE2 | TYR | A | 676 | 20.354 | 0.962 | 35.097 | 1.00 | 27.90 |
| ATOM | 1125 | CZ | TYR | A | 676 | 19.443 | 1.117 | 36.118 | 1.00 | 24.78 |
| ATOM | 1126 | OH | TYR | A | 676 | 18.866 | 2.354 | 36.326 | 1.00 | 28.03 |
| ATOM | 1127 | C | TYR | A | 676 | 22.605 | −4.623 | 36.471 | 1.00 | 16.48 |
| ATOM | 1128 | O | TYR | A | 676 | 21.949 | −5.478 | 35.872 | 1.00 | 15.88 |
| ATOM | 1129 | N | ASN | A | 677 | 23.814 | −4.863 | 36.968 | 1.00 | 16.14 |
| ATOM | 1130 | CA | ASN | A | 677 | 24.433 | −6.182 | 36.888 | 1.00 | 14.23 |
| ATOM | 1131 | CB | ASN | A | 677 | 23.666 | −7.138 | 37.812 | 1.00 | 21.33 |
| ATOM | 1132 | CG | ASN | A | 677 | 24.382 | −8.452 | 38.022 | 1.00 | 29.37 |
| ATOM | 1133 | OD1 | ASN | A | 677 | 25.570 | −8.478 | 38.349 | 1.00 | 38.44 |
| ATOM | 1134 | ND2 | ASN | A | 677 | 23.662 | −9.555 | 37.847 | 1.00 | 42.62 |
| ATOM | 1135 | C | ASN | A | 677 | 24.516 | −6.765 | 35.474 | 1.00 | 15.05 |
| ATOM | 1136 | O | ASN | A | 677 | 24.332 | −7.966 | 35.275 | 1.00 | 14.59 |
| ATOM | 1137 | N | ASP | A | 678 | 24.802 | −5.904 | 34.502 | 1.00 | 13.69 |
| ATOM | 1138 | CA | ASP | A | 678 | 24.926 | −6.302 | 33.100 | 1.00 | 16.08 |
| ATOM | 1139 | CB | ASP | A | 678 | 26.080 | −7.293 | 32.915 | 1.00 | 12.96 |
| ATOM | 1140 | CG | ASP | A | 678 | 27.430 | −6.691 | 33.234 | 1.00 | 18.27 |
| ATOM | 1141 | OD1 | ASP | A | 678 | 27.519 | −5.452 | 33.377 | 1.00 | 16.21 |
| ATOM | 1142 | OD2 | ASP | A | 678 | 28.406 | −7.464 | 33.334 | 1.00 | 20.95 |
| ATOM | 1143 | C | ASP | A | 678 | 23.667 | −6.919 | 32.499 | 1.00 | 16.35 |
| ATOM | 1144 | O | ASP | A | 678 | 23.731 | −7.509 | 31.420 | 1.00 | 17.49 |
| ATOM | 1145 | N | GLU | A | 679 | 22.534 | −6.790 | 33.184 | 1.00 | 14.03 |
| ATOM | 1146 | CA | GLU | A | 679 | 21.278 | −7.358 | 32.690 | 1.00 | 14.39 |
| ATOM | 1147 | CB | GLU | A | 679 | 20.567 | −8.133 | 33.799 | 1.00 | 14.04 |
| ATOM | 1148 | CG | GLU | A | 679 | 21.314 | −9.351 | 34.300 | 1.00 | 22.79 |
| ATOM | 1149 | CD | GLU | A | 679 | 20.514 | −10.128 | 35.330 | 1.00 | 43.42 |
| ATOM | 1150 | OE1 | GLU | A | 679 | 19.464 | −10.699 | 34.964 | 1.00 | 51.30 |
| ATOM | 1151 | OE2 | GLU | A | 679 | 20.931 | −10.162 | 36.507 | 1.00 | 41.34 |
| ATOM | 1152 | C | GLU | A | 679 | 20.320 | −6.301 | 32.151 | 1.00 | 12.89 |
| ATOM | 1153 | O | GLU | A | 679 | 19.873 | −5.430 | 32.895 | 1.00 | 13.61 |
| ATOM | 1154 | N | SER | A | 680 | 19.993 | −6.401 | 30.863 | 1.00 | 10.87 |
| ATOM | 1155 | CA | SER | A | 680 | 19.086 | −5.456 | 30.209 | 1.00 | 12.50 |
| ATOM | 1156 | CB | SER | A | 680 | 17.627 | −5.787 | 30.555 | 1.00 | 12.08 |
| ATOM | 1157 | OG | SER | A | 680 | 17.267 | −7.087 | 30.111 | 1.00 | 11.26 |
| ATOM | 1158 | C | SER | A | 680 | 19.409 | −4.038 | 30.663 | 1.00 | 11.78 |
| ATOM | 1159 | O | SER | A | 680 | 18.517 | −3.280 | 31.045 | 1.00 | 12.76 |
| ATOM | 1160 | N | VAL | A | 681 | 20.693 | −3.693 | 30.617 | 1.00 | 9.67 |
| ATOM | 1161 | CA | VAL | A | 681 | 21.183 | −2.386 | 31.049 | 1.00 | 7.19 |
| ATOM | 1162 | CB | VAL | A | 681 | 22.688 | −2.248 | 30.725 | 1.00 | 11.97 |
| ATOM | 1163 | CG1 | VAL | A | 681 | 23.219 | −0.917 | 31.245 | 1.00 | 10.98 |
| ATOM | 1164 | CG2 | VAL | A | 681 | 23.460 | −3.423 | 31.346 | 1.00 | 12.93 |
| ATOM | 1165 | C | VAL | A | 681 | 20.426 | −1.195 | 30.453 | 1.00 | 9.58 |
| ATOM | 1166 | O | VAL | A | 681 | 19.870 | −0.378 | 31.185 | 1.00 | 10.02 |
| ATOM | 1167 | N | LEU | A | 681A | 20.420 | −1.094 | 29.128 | 1.00 | 9.51 |
| ATOM | 1168 | CA | LEU | A | 681A | 19.718 | −0.011 | 28.443 | 1.00 | 10.16 |
| ATOM | 1169 | CB | LEU | A | 681A | 19.773 | −0.216 | 26.929 | 1.00 | 7.24 |
| ATOM | 1170 | CG | LEU | A | 681A | 21.079 | 0.035 | 26.178 | 1.00 | 9.74 |
| ATOM | 1171 | CD1 | LEU | A | 681A | 20.979 | −0.563 | 24.788 | 1.00 | 13.02 |
| ATOM | 1172 | CD2 | LEU | A | 681A | 21.345 | 1.526 | 26.102 | 1.00 | 15.84 |
| ATOM | 1173 | C | LEU | A | 681A | 18.253 | 0.049 | 28.851 | 1.00 | 8.34 |
| ATOM | 1174 | O | LEU | A | 681A | 17.725 | 1.118 | 29.174 | 1.00 | 10.62 |
| ATOM | 1175 | N | GLU | A | 682 | 17.599 | −1.110 | 28.834 | 1.00 | 10.08 |
| ATOM | 1176 | CA | GLU | A | 682 | 16.183 | −1.191 | 29.161 | 1.00 | 7.57 |
| ATOM | 1177 | CB | GLU | A | 682 | 15.667 | −2.599 | 28.855 | 1.00 | 6.60 |
| ATOM | 1178 | CG | GLU | A | 682 | 15.731 | −2.954 | 27.348 | 1.00 | 8.78 |
| ATOM | 1179 | CD | GLU | A | 682 | 17.151 | −3.199 | 26.835 | 1.00 | 12.12 |
| ATOM | 1180 | OE1 | GLU | A | 682 | 18.001 | −3.693 | 27.614 | 1.00 | 10.38 |
| ATOM | 1181 | OE2 | GLU | A | 682 | 17.418 | −2.919 | 25.646 | 1.00 | 10.27 |
| ATOM | 1182 | C | GLU | A | 682 | 15.867 | −0.773 | 30.594 | 1.00 | 8.81 |
| ATOM | 1183 | O | GLU | A | 682 | 14.829 | −0.158 | 30.854 | 1.00 | 7.94 |
| ATOM | 1184 | N | HIS | A | 683 | 16.752 | −1.099 | 31.526 | 1.00 | 9.05 |
| ATOM | 1185 | CA | HIS | A | 683 | 16.531 | −0.677 | 32.897 | 1.00 | 10.40 |
| ATOM | 1186 | CB | HIS | A | 683 | 17.561 | −1.324 | 33.830 | 1.00 | 11.92 |
| ATOM | 1187 | CG | HIS | A | 683 | 17.161 | −2.686 | 34.296 | 1.00 | 10.38 |
| ATOM | 1188 | CD2 | HIS | A | 683 | 17.557 | −3.920 | 33.903 | 1.00 | 9.93 |
| ATOM | 1189 | ND1 | HIS | A | 683 | 16.172 | −2.888 | 35.238 | 1.00 | 14.33 |
| ATOM | 1190 | CE1 | HIS | A | 683 | 15.977 | −4.183 | 35.400 | 1.00 | 7.54 |
| ATOM | 1191 | NE2 | HIS | A | 683 | 16.806 | −4.831 | 34.599 | 1.00 | 15.67 |
| ATOM | 1192 | C | HIS | A | 683 | 16.631 | 0.846 | 32.925 | 1.00 | 11.08 |
| ATOM | 1193 | O | HIS | A | 683 | 15.868 | 1.512 | 33.621 | 1.00 | 10.48 |
| ATOM | 1194 | N | HIS | A | 684 | 17.553 | 1.397 | 32.138 | 1.00 | 10.86 |
| ATOM | 1195 | CA | HIS | A | 684 | 17.710 | 2.844 | 32.076 | 1.00 | 9.98 |
| ATOM | 1196 | CB | HIS | A | 684 | 18.941 | 3.233 | 31.256 | 1.00 | 9.09 |
| ATOM | 1197 | CG | HIS | A | 684 | 19.229 | 4.697 | 31.284 | 1.00 | 8.54 |
| ATOM | 1198 | CD2 | HIS | A | 684 | 19.399 | 5.555 | 32.322 | 1.00 | 5.20 |
| ATOM | 1199 | ND1 | HIS | A | 684 | 19.313 | 5.467 | 30.142 | 1.00 | 17.15 |
| ATOM | 1200 | CE1 | HIS | A | 684 | 19.518 | 6.728 | 30.474 | 1.00 | 9.30 |
| ATOM | 1201 | NE2 | HIS | A | 684 | 19.573 | 6.807 | 31.795 | 1.00 | 15.79 |
| ATOM | 1202 | C | HIS | A | 684 | 16.473 | 3.502 | 31.465 | 1.00 | 9.44 |
| ATOM | 1203 | O | HIS | A | 684 | 16.020 | 4.542 | 31.941 | 1.00 | 11.75 |
| ATOM | 1204 | N | HIS | A | 685 | 15.929 | 2.898 | 30.411 | 1.00 | 9.42 |
| ATOM | 1205 | CA | HIS | A | 685 | 14.740 | 3.447 | 29.757 | 1.00 | 7.57 |
| ATOM | 1206 | CB | HIS | A | 685 | 14.361 | 2.621 | 28.521 | 1.00 | 14.86 |
| ATOM | 1207 | CG | HIS | A | 685 | 15.472 | 2.468 | 27.521 | 1.00 | 7.24 |
| ATOM | 1208 | CD2 | HIS | A | 685 | 15.846 | 1.402 | 26.773 | 1.00 | 9.32 |
| ATOM | 1209 | ND1 | HIS | A | 685 | 16.299 | 3.506 | 27.161 | 1.00 | 8.86 |
| ATOM | 1210 | CE1 | HIS | A | 685 | 17.141 | 3.088 | 26.227 | 1.00 | 14.03 |
| ATOM | 1211 | NE2 | HIS | A | 685 | 16.886 | 1.819 | 25.974 | 1.00 | 8.62 |
| ATOM | 1212 | C | HIS | A | 685 | 13.574 | 3.446 | 30.747 | 1.00 | 9.27 |
| ATOM | 1213 | O | HIS | A | 685 | 12.798 | 4.401 | 30.805 | 1.00 | 9.78 |
| ATOM | 1214 | N | PHE | A | 686 | 13.439 | 2.376 | 31.523 | 1.00 | 8.10 |
| ATOM | 1215 | CA | PHE | A | 686 | 12.351 | 2.314 | 32.507 | 1.00 | 10.05 |
| ATOM | 1216 | CB | PHE | A | 686 | 12.291 | 0.923 | 33.151 | 1.00 | 8.82 |
| ATOM | 1217 | CG | PHE | A | 686 | 11.281 | 0.808 | 34.263 | 1.00 | 10.55 |
| ATOM | 1218 | CD1 | PHE | A | 686 | 9.937 | 1.078 | 34.035 | 1.00 | 10.53 |
| ATOM | 1219 | CD2 | PHE | A | 686 | 11.674 | 0.442 | 35.538 | 1.00 | 11.42 |
| ATOM | 1220 | CE1 | PHE | A | 686 | 9.013 | 0.985 | 35.061 | 1.00 | 14.12 |
| ATOM | 1221 | CE2 | PHE | A | 686 | 10.754 | 0.348 | 36.568 | 1.00 | 15.20 |
| ATOM | 1222 | CZ | PHE | A | 686 | 9.423 | 0.618 | 36.328 | 1.00 | 13.66 |
| ATOM | 1223 | C | PHE | A | 686 | 12.554 | 3.391 | 33.570 | 1.00 | 9.59 |
| ATOM | 1224 | O | PHE | A | 686 | 11.599 | 4.036 | 34.022 | 1.00 | 11.10 |
| ATOM | 1225 | N | ASP | A | 687 | 13.807 | 3.603 | 33.965 | 1.00 | 10.38 |
| ATOM | 1226 | CA | ASP | A | 687 | 14.169 | 4.620 | 34.950 | 1.00 | 13.63 |
| ATOM | 1227 | CB | ASP | A | 687 | 15.699 | 4.646 | 35.147 | 1.00 | 15.63 |
| ATOM | 1228 | CG | ASP | A | 687 | 16.163 | 5.810 | 36.032 | 1.00 | 35.43 |
| ATOM | 1229 | OD1 | ASP | A | 687 | 15.842 | 5.812 | 37.238 | 1.00 | 39.21 |
| ATOM | 1230 | OD2 | ASP | A | 687 | 16.855 | 6.721 | 35.514 | 1.00 | 46.67 |
| ATOM | 1231 | C | ASP | A | 687 | 13.701 | 5.983 | 34.453 | 1.00 | 11.19 |
| ATOM | 1232 | O | ASP | A | 687 | 13.079 | 6.742 | 35.191 | 1.00 | 12.63 |
| ATOM | 1233 | N | GLN | A | 688 | 14.026 | 6.266 | 33.194 | 1.00 | 10.97 |
| ATOM | 1234 | CA | GLN | A | 688 | 13.664 | 7.531 | 32.571 | 1.00 | 10.87 |
| ATOM | 1235 | CB | GLN | A | 688 | 14.331 | 7.623 | 31.193 | 1.00 | 15.23 |
| ATOM | 1236 | CG | GLN | A | 688 | 15.844 | 7.876 | 31.260 | 1.00 | 11.96 |
| ATOM | 1237 | CD | GLN | A | 688 | 16.179 | 9.350 | 31.456 | 1.00 | 14.39 |
| ATOM | 1238 | OE1 | GLN | A | 688 | 15.805 | 10.193 | 30.640 | 1.00 | 20.66 |
| ATOM | 1239 | NE2 | GLN | A | 688 | 16.881 | 9.663 | 32.535 | 1.00 | 29.50 |
| ATOM | 1240 | C | GLN | A | 688 | 12.158 | 7.651 | 32.455 | 1.00 | 10.36 |
| ATOM | 1241 | O | GLN | A | 688 | 11.587 | 8.729 | 32.641 | 1.00 | 10.53 |
| ATOM | 1242 | N | CYS | A | 689 | 11.495 | 6.536 | 32.160 | 1.00 | 8.48 |
| ATOM | 1243 | CA | CYS | A | 689 | 10.043 | 6.517 | 32.040 | 1.00 | 9.59 |
| ATOM | 1244 | CB | CYS | A | 689 | 9.558 | 5.097 | 31.706 | 1.00 | 11.54 |
| ATOM | 1245 | SG | CYS | A | 689 | 7.772 | 4.940 | 31.573 | 1.00 | 11.98 |
| ATOM | 1246 | C | CYS | A | 689 | 9.406 | 7.005 | 33.343 | 1.00 | 10.54 |
| ATOM | 1247 | O | CYS | A | 689 | 8.559 | 7.905 | 33.331 | 1.00 | 10.86 |
| ATOM | 1248 | N | LEU | A | 690 | 9.822 | 6.417 | 34.463 | 1.00 | 12.43 |
| ATOM | 1249 | CA | LEU | A | 690 | 9.316 | 6.786 | 35.787 | 1.00 | 13.70 |
| ATOM | 1250 | CB | LEU | A | 690 | 9.968 | 5.912 | 36.863 | 1.00 | 15.35 |
| ATOM | 1251 | CG | LEU | A | 690 | 9.620 | 4.423 | 36.853 | 1.00 | 18.35 |
| ATOM | 1252 | CD1 | LEU | A | 690 | 10.326 | 3.723 | 38.008 | 1.00 | 25.25 |
| ATOM | 1253 | CD2 | LEU | A | 690 | 8.118 | 4.248 | 36.969 | 1.00 | 17.06 |
| ATOM | 1254 | C | LEU | A | 690 | 9.605 | 8.247 | 36.093 | 1.00 | 14.05 |
| ATOM | 1255 | O | LEU | A | 690 | 8.767 | 8.975 | 36.633 | 1.00 | 15.63 |
| ATOM | 1256 | N | MET | A | 691 | 10.817 | 8.681 | 35.750 | 1.00 | 13.87 |
| ATOM | 1257 | CA | MET | A | 691 | 11.256 | 10.042 | 35.975 | 1.00 | 14.41 |
| ATOM | 1258 | CB | MET | A | 691 | 12.676 | 10.212 | 35.411 | 1.00 | 14.62 |
| ATOM | 1259 | CG | MET | A | 691 | 13.371 | 11.496 | 35.757 | 1.00 | 32.36 |
| ATOM | 1260 | SD | MET | A | 691 | 12.902 | 12.755 | 34.635 | 1.00 | 45.55 |
| ATOM | 1261 | CE | MET | A | 691 | 13.918 | 12.471 | 33.200 | 1.00 | 37.72 |
| ATOM | 1262 | C | MET | A | 691 | 10.285 | 11.024 | 35.336 | 1.00 | 13.72 |
| ATOM | 1263 | O | MET | A | 691 | 9.863 | 11.990 | 35.971 | 1.00 | 14.45 |
| ATOM | 1264 | N | ILE | A | 692 | 9.903 | 10.758 | 34.091 | 1.00 | 10.48 |
| ATOM | 1265 | CA | ILE | A | 692 | 8.980 | 11.644 | 33.400 | 1.00 | 9.98 |
| ATOM | 1266 | CB | ILE | A | 692 | 8.966 | 11.371 | 31.895 | 1.00 | 10.00 |
| ATOM | 1267 | CG2 | ILE | A | 692 | 7.909 | 12.240 | 31.224 | 1.00 | 12.51 |
| ATOM | 1268 | CG1 | ILE | A | 692 | 10.346 | 11.691 | 31.315 | 1.00 | 14.40 |
| ATOM | 1269 | CD1 | ILE | A | 692 | 10.482 | 11.400 | 29.841 | 1.00 | 19.81 |
| ATOM | 1270 | C | ILE | A | 692 | 7.578 | 11.534 | 33.973 | 1.00 | 10.09 |
| ATOM | 1271 | O | ILE | A | 692 | 6.906 | 12.547 | 34.166 | 1.00 | 11.41 |
| ATOM | 1272 | N | LEU | A | 693 | 7.149 | 10.316 | 34.288 | 1.00 | 12.13 |
| ATOM | 1273 | CA | LEU | A | 693 | 5.821 | 10.111 | 34.862 | 1.00 | 13.85 |
| ATOM | 1274 | CB | LEU | A | 693 | 5.548 | 8.615 | 35.058 | 1.00 | 13.96 |
| ATOM | 1275 | CG | LEU | A | 693 | 5.212 | 7.826 | 33.789 | 1.00 | 10.33 |
| ATOM | 1276 | CD1 | LEU | A | 693 | 5.183 | 6.335 | 34.082 | 1.00 | 17.28 |
| ATOM | 1277 | CD2 | LEU | A | 693 | 3.868 | 8.305 | 33.243 | 1.00 | 9.40 |
| ATOM | 1278 | C | LEU | A | 693 | 5.644 | 10.837 | 36.193 | 1.00 | 14.19 |
| ATOM | 1279 | O | LEU | A | 693 | 4.524 | 11.179 | 36.577 | 1.00 | 16.64 |
| ATOM | 1280 | N | ASN | A | 694 | 6.747 | 11.073 | 36.893 | 1.00 | 16.46 |
| ATOM | 1281 | CA | ASN | A | 694 | 6.697 | 11.755 | 38.183 | 1.00 | 16.63 |
| ATOM | 1282 | CB | ASN | A | 694 | 7.686 | 11.116 | 39.159 | 1.00 | 20.12 |
| ATOM | 1283 | CG | ASN | A | 694 | 7.231 | 9.760 | 39.641 | 1.00 | 24.99 |
| ATOM | 1284 | OD1 | ASN | A | 694 | 7.869 | 8.742 | 39.373 | 1.00 | 30.33 |
| ATOM | 1285 | ND2 | ASN | A | 694 | 6.115 | 9.736 | 40.360 | 1.00 | 25.93 |
| ATOM | 1286 | C | ASN | A | 694 | 6.989 | 13.247 | 38.101 | 1.00 | 18.31 |
| ATOM | 1287 | O | ASN | A | 694 | 6.855 | 13.958 | 39.093 | 1.00 | 18.26 |
| ATOM | 1288 | N | SER | A | 695 | 7.395 | 13.721 | 36.930 | 1.00 | 16.05 |
| ATOM | 1289 | CA | SER | A | 695 | 7.704 | 15.139 | 36.755 | 1.00 | 17.70 |
| ATOM | 1290 | CB | SER | A | 695 | 8.321 | 15.376 | 35.376 | 1.00 | 19.30 |
| ATOM | 1291 | OG | SER | A | 695 | 9.607 | 14.793 | 35.298 | 1.00 | 34.04 |
| ATOM | 1292 | C | SER | A | 695 | 6.471 | 16.022 | 36.914 | 1.00 | 15.83 |
| ATOM | 1293 | O | SER | A | 695 | 5.400 | 15.710 | 36.406 | 1.00 | 17.46 |
| ATOM | 1294 | N | PRO | A | 696 | 6.611 | 17.144 | 37.630 | 1.00 | 18.97 |
| ATOM | 1295 | CD | PRO | A | 696 | 7.779 | 17.577 | 38.415 | 1.00 | 26.97 |
| ATOM | 1296 | CA | PRO | A | 696 | 5.480 | 18.053 | 37.830 | 1.00 | 18.99 |
| ATOM | 1297 | CB | PRO | A | 696 | 6.113 | 19.220 | 38.579 | 1.00 | 22.14 |
| ATOM | 1298 | CG | PRO | A | 696 | 7.154 | 18.534 | 39.414 | 1.00 | 28.71 |
| ATOM | 1299 | C | PRO | A | 696 | 4.866 | 18.480 | 36.498 | 1.00 | 16.02 |
| ATOM | 1300 | O | PRO | A | 696 | 5.585 | 18.839 | 35.564 | 1.00 | 18.08 |
| ATOM | 1301 | N | GLY | A | 697 | 3.539 | 18.425 | 36.418 | 1.00 | 16.17 |
| ATOM | 1302 | CA | GLY | A | 697 | 2.843 | 18.804 | 35.201 | 1.00 | 16.65 |
| ATOM | 1303 | C | GLY | A | 697 | 2.862 | 17.725 | 34.130 | 1.00 | 14.72 |
| ATOM | 1304 | O | GLY | A | 697 | 2.283 | 17.901 | 33.060 | 1.00 | 15.86 |
| ATOM | 1305 | N | ASN | A | 698 | 3.511 | 16.601 | 34.424 | 1.00 | 14.68 |
| ATOM | 1306 | CA | ASN | A | 698 | 3.621 | 15.493 | 33.471 | 1.00 | 14.66 |
| ATOM | 1307 | CB | ASN | A | 698 | 5.099 | 15.209 | 33.174 | 1.00 | 15.25 |
| ATOM | 1308 | CG | ASN | A | 698 | 5.760 | 16.304 | 32.365 | 1.00 | 19.94 |
| ATOM | 1309 | OD1 | ASN | A | 698 | 5.883 | 16.199 | 31.146 | 1.00 | 13.89 |
| ATOM | 1310 | ND2 | ASN | A | 698 | 6.186 | 17.367 | 33.039 | 1.00 | 16.33 |
| ATOM | 1311 | C | ASN | A | 698 | 2.990 | 14.198 | 33.973 | 1.00 | 14.82 |
| ATOM | 1312 | O | ASN | A | 698 | 3.044 | 13.174 | 33.293 | 1.00 | 13.20 |
| ATOM | 1313 | N | GLN | A | 699 | 2.379 | 14.243 | 35.151 | 1.00 | 13.46 |
| ATOM | 1314 | CA | GLN | A | 699 | 1.807 | 13.041 | 35.749 | 1.00 | 14.53 |
| ATOM | 1315 | CB | GLN | A | 699 | 1.652 | 13.281 | 37.251 | 1.00 | 16.25 |
| ATOM | 1316 | CG | GLN | A | 699 | 2.957 | 13.713 | 37.902 | 1.00 | 17.08 |
| ATOM | 1317 | CD | GLN | A | 699 | 2.787 | 14.103 | 39.350 | 1.00 | 37.15 |
| ATOM | 1318 | OE1 | GLN | A | 699 | 2.147 | 15.105 | 39.661 | 1.00 | 41.13 |
| ATOM | 1319 | NE2 | GLN | A | 699 | 3.357 | 13.307 | 40.248 | 1.00 | 41.91 |
| ATOM | 1320 | C | GLN | A | 699 | 0.499 | 12.522 | 35.158 | 1.00 | 13.96 |
| ATOM | 1321 | O | GLN | A | 699 | −0.567 | 12.676 | 35.754 | 1.00 | 15.67 |
| ATOM | 1322 | N | ILE | A | 700 | 0.582 | 11.867 | 34.003 | 1.00 | 13.44 |
| ATOM | 1323 | CA | ILE | A | 700 | −0.617 | 11.348 | 33.356 | 1.00 | 14.25 |
| ATOM | 1324 | CB | ILE | A | 700 | −0.345 | 10.974 | 31.881 | 1.00 | 15.57 |
| ATOM | 1325 | CG2 | ILE | A | 700 | 0.010 | 12.222 | 31.086 | 1.00 | 15.78 |
| ATOM | 1326 | CG1 | ILE | A | 700 | 0.777 | 9.941 | 31.793 | 1.00 | 16.74 |
| ATOM | 1327 | CD1 | ILE | A | 700 | 0.940 | 9.361 | 30.407 | 1.00 | 12.98 |
| ATOM | 1328 | C | ILE | A | 700 | −1.262 | 10.151 | 34.064 | 1.00 | 13.88 |
| ATOM | 1329 | O | ILE | A | 700 | −2.342 | 9.709 | 33.673 | 1.00 | 14.53 |
| ATOM | 1330 | N | LEU | A | 701 | −0.609 | 9.624 | 35.098 | 1.00 | 11.60 |
| ATOM | 1331 | CA | LEU | A | 701 | −1.169 | 8.495 | 35.845 | 1.00 | 14.58 |
| ATOM | 1332 | CB | LEU | A | 701 | −0.150 | 7.358 | 35.965 | 1.00 | 13.85 |
| ATOM | 1333 | CG | LEU | A | 701 | 0.310 | 6.673 | 34.677 | 1.00 | 11.07 |
| ATOM | 1334 | CD1 | LEU | A | 701 | 1.270 | 5.539 | 35.031 | 1.00 | 13.34 |
| ATOM | 1335 | CD2 | LEU | A | 701 | −0.895 | 6.128 | 33.921 | 1.00 | 12.89 |
| ATOM | 1336 | C | LEU | A | 701 | −1.612 | 8.914 | 37.247 | 1.00 | 13.75 |
| ATOM | 1337 | O | LEU | A | 701 | −2.053 | 8.084 | 38.040 | 1.00 | 15.74 |
| ATOM | 1338 | N | SER | A | 702 | −1.497 | 10.201 | 37.547 | 1.00 | 15.74 |
| ATOM | 1339 | CA | SER | A | 702 | −1.872 | 10.717 | 38.860 | 1.00 | 18.36 |
| ATOM | 1340 | CB | SER | A | 702 | −1.573 | 12.217 | 38.949 | 1.00 | 20.19 |
| ATOM | 1341 | OG | SER | A | 702 | −2.446 | 12.958 | 38.113 | 1.00 | 21.94 |
| ATOM | 1342 | C | SER | A | 702 | −3.347 | 10.490 | 39.176 | 1.00 | 19.34 |
| ATOM | 1343 | O | SER | A | 702 | −3.754 | 10.559 | 40.336 | 1.00 | 20.75 |
| ATOM | 1344 | N | GLY | A | 703 | −4.144 | 10.224 | 38.148 | 1.00 | 18.92 |
| ATOM | 1345 | CA | GLY | A | 703 | −5.565 | 10.012 | 38.359 | 1.00 | 20.40 |
| ATOM | 1346 | C | GLY | A | 703 | −5.944 | 8.600 | 38.764 | 1.00 | 21.19 |
| ATOM | 1347 | O | GLY | A | 703 | −7.036 | 8.369 | 39.284 | 1.00 | 20.43 |
| ATOM | 1348 | N | LEU | A | 704 | −5.045 | 7.649 | 38.539 | 1.00 | 16.00 |
| ATOM | 1349 | CA | LEU | A | 704 | −5.326 | 6.259 | 38.876 | 1.00 | 15.59 |
| ATOM | 1350 | CB | LEU | A | 704 | −4.262 | 5.335 | 38.278 | 1.00 | 9.29 |
| ATOM | 1351 | CG | LEU | A | 704 | −4.459 | 4.921 | 36.823 | 1.00 | 14.10 |
| ATOM | 1352 | CD1 | LEU | A | 704 | −4.510 | 6.143 | 35.926 | 1.00 | 17.73 |
| ATOM | 1353 | CD2 | LEU | A | 704 | −3.312 | 3.998 | 36.418 | 1.00 | 11.19 |
| ATOM | 1354 | C | LEU | A | 704 | −5.419 | 5.993 | 40.368 | 1.00 | 16.40 |
| ATOM | 1355 | O | LEU | A | 704 | −4.773 | 6.659 | 41.178 | 1.00 | 16.88 |
| ATOM | 1356 | N | SER | A | 705 | −6.233 | 5.004 | 40.721 | 1.00 | 15.43 |
| ATOM | 1357 | CA | SER | A | 705 | −6.395 | 4.621 | 42.111 | 1.00 | 15.96 |
| ATOM | 1358 | CB | SER | A | 705 | −7.556 | 3.640 | 42.268 | 1.00 | 15.57 |
| ATOM | 1359 | OG | SER | A | 705 | −7.242 | 2.402 | 41.658 | 1.00 | 15.44 |
| ATOM | 1360 | C | SER | A | 705 | −5.092 | 3.947 | 42.515 | 1.00 | 16.32 |
| ATOM | 1361 | O | SER | A | 705 | −4.280 | 3.583 | 41.662 | 1.00 | 17.25 |
| ATOM | 1362 | N | ILE | A | 706 | −4.886 | 3.782 | 43.812 | 1.00 | 17.56 |
| ATOM | 1363 | CA | ILE | A | 706 | −3.666 | 3.164 | 44.292 | 1.00 | 16.88 |
| ATOM | 1364 | CB | ILE | A | 706 | −3.679 | 3.086 | 45.836 | 1.00 | 21.33 |
| ATOM | 1365 | CG2 | ILE | A | 706 | −2.466 | 2.331 | 46.335 | 1.00 | 23.53 |
| ATOM | 1366 | CG1 | ILE | A | 706 | −3.712 | 4.507 | 46.411 | 1.00 | 21.96 |
| ATOM | 1367 | CD1 | ILE | A | 706 | −3.892 | 4.577 | 47.918 | 1.00 | 22.24 |
| ATOM | 1368 | C | ILE | A | 706 | −3.415 | 1.782 | 43.685 | 1.00 | 16.48 |
| ATOM | 1369 | O | ILE | A | 706 | −2.291 | 1.470 | 43.291 | 1.00 | 18.62 |
| ATOM | 1370 | N | GLU | A | 707 | −4.456 | 0.962 | 43.584 | 1.00 | 15.92 |
| ATOM | 1371 | CA | GLU | A | 707 | −4.304 | −0.376 | 43.019 | 1.00 | 14.53 |
| ATOM | 1372 | CB | GLU | A | 707 | −5.509 | −1.250 | 43.378 | 1.00 | 19.33 |
| ATOM | 1373 | CG | GLU | A | 707 | −5.518 | −1.682 | 44.836 | 1.00 | 24.82 |
| ATOM | 1374 | CD | GLU | A | 707 | −4.269 | −2.460 | 45.214 | 1.00 | 31.26 |
| ATOM | 1375 | OE1 | GLU | A | 707 | −4.029 | −3.525 | 44.610 | 1.00 | 34.47 |
| ATOM | 1376 | OE2 | GLU | A | 707 | −3.525 | −2.006 | 46.110 | 1.00 | 30.79 |
| ATOM | 1377 | C | GLU | A | 707 | −4.093 | −0.385 | 41.507 | 1.00 | 14.27 |
| ATOM | 1378 | O | GLU | A | 707 | −3.283 | −1.156 | 41.002 | 1.00 | 15.02 |
| ATOM | 1379 | N | GLU | A | 708 | −4.818 | 0.458 | 40.782 | 1.00 | 11.81 |
| ATOM | 1380 | CA | GLU | A | 708 | −4.644 | 0.495 | 39.337 | 1.00 | 12.49 |
| ATOM | 1381 | CB | GLU | A | 708 | −5.756 | 1.317 | 38.673 | 1.00 | 14.73 |
| ATOM | 1382 | CG | GLU | A | 708 | −7.134 | 0.663 | 38.813 | 1.00 | 11.98 |
| ATOM | 1383 | CD | GLU | A | 708 | −8.088 | 1.020 | 37.683 | 1.00 | 18.01 |
| ATOM | 1384 | OE1 | GLU | A | 708 | −8.121 | 2.196 | 37.274 | 1.00 | 19.76 |
| ATOM | 1385 | OE2 | GLU | A | 708 | −8.816 | 0.120 | 37.216 | 1.00 | 22.22 |
| ATOM | 1386 | C | GLU | A | 708 | −3.264 | 1.054 | 38.996 | 1.00 | 14.00 |
| ATOM | 1387 | O | GLU | A | 708 | −2.624 | 0.607 | 38.047 | 1.00 | 12.79 |
| ATOM | 1388 | N | TYR | A | 709 | −2.798 | 2.018 | 39.784 | 1.00 | 12.70 |
| ATOM | 1389 | CA | TYR | A | 709 | −1.482 | 2.604 | 39.551 | 1.00 | 13.42 |
| ATOM | 1390 | CB | TYR | A | 709 | −1.204 | 3.724 | 40.557 | 1.00 | 12.05 |
| ATOM | 1391 | CG | TYR | A | 709 | 0.129 | 4.407 | 40.339 | 1.00 | 19.13 |
| ATOM | 1392 | CD1 | TYR | A | 709 | 0.311 | 5.302 | 39.296 | 1.00 | 14.81 |
| ATOM | 1393 | CE1 | TYR | A | 709 | 1.530 | 5.928 | 39.091 | 1.00 | 15.81 |
| ATOM | 1394 | CD2 | TYR | A | 709 | 1.207 | 4.149 | 41.174 | 1.00 | 17.39 |
| ATOM | 1395 | CE2 | TYR | A | 709 | 2.427 | 4.767 | 40.978 | 1.00 | 18.97 |
| ATOM | 1396 | CZ | TYR | A | 709 | 2.583 | 5.658 | 39.934 | 1.00 | 17.67 |
| ATOM | 1397 | OH | TYR | A | 709 | 3.793 | 6.284 | 39.737 | 1.00 | 18.77 |
| ATOM | 1398 | C | TYR | A | 709 | −0.424 | 1.515 | 39.708 | 1.00 | 15.44 |
| ATOM | 1399 | O | TYR | A | 709 | 0.452 | 1.348 | 38.855 | 1.00 | 15.06 |
| ATOM | 1400 | N | LYS | A | 710 | −0.520 | 0.774 | 40.808 | 1.00 | 15.25 |
| ATOM | 1401 | CA | LYS | A | 710 | 0.409 | −0.311 | 41.095 | 1.00 | 15.31 |
| ATOM | 1402 | CB | LYS | A | 710 | −0.020 | −1.026 | 42.379 | 1.00 | 22.10 |
| ATOM | 1403 | CG | LYS | A | 710 | 0.809 | −2.249 | 42.716 | 1.00 | 23.74 |
| ATOM | 1404 | CD | LYS | A | 710 | 0.299 | −2.931 | 43.978 | 1.00 | 33.24 |
| ATOM | 1405 | CE | LYS | A | 710 | 0.996 | −4.263 | 44.204 | 1.00 | 39.08 |
| ATOM | 1406 | NZ | LYS | A | 710 | 2.478 | −4.117 | 44.248 | 1.00 | 42.37 |
| ATOM | 1407 | C | LYS | A | 710 | 0.474 | −1.309 | 39.939 | 1.00 | 14.94 |
| ATOM | 1408 | O | LYS | A | 710 | 1.555 | −1.645 | 39.448 | 1.00 | 12.87 |
| ATOM | 1409 | N | THR | A | 711 | −0.692 | −1.777 | 39.506 | 1.00 | 13.50 |
| ATOM | 1410 | CA | THR | A | 711 | −0.783 | −2.736 | 38.411 | 1.00 | 12.45 |
| ATOM | 1411 | CB | THR | A | 711 | −2.249 | −3.092 | 38.128 | 1.00 | 17.44 |
| ATOM | 1412 | OG1 | THR | A | 711 | −2.791 | −3.792 | 39.255 | 1.00 | 21.96 |
| ATOM | 1413 | CG2 | THR | A | 711 | −2.365 | −3.953 | 36.872 | 1.00 | 16.75 |
| ATOM | 1414 | C | THR | A | 711 | −0.161 | −2.198 | 37.129 | 1.00 | 11.72 |
| ATOM | 1415 | O | THR | A | 711 | 0.597 | −2.896 | 36.442 | 1.00 | 13.19 |
| ATOM | 1416 | N | THR | A | 712 | −0.487 | −0.954 | 36.809 | 1.00 | 11.15 |
| ATOM | 1417 | CA | THR | A | 712 | 0.015 | −0.319 | 35.599 | 1.00 | 13.99 |
| ATOM | 1418 | CB | THR | A | 712 | −0.598 | 1.081 | 35.424 | 1.00 | 9.97 |
| ATOM | 1419 | OG1 | THR | A | 712 | −2.029 | 0.967 | 35.357 | 1.00 | 14.40 |
| ATOM | 1420 | CG2 | THR | A | 712 | −0.088 | 1.729 | 34.133 | 1.00 | 12.92 |
| ATOM | 1421 | C | THR | A | 712 | 1.541 | −0.221 | 35.556 | 1.00 | 12.77 |
| ATOM | 1422 | O | THR | A | 712 | 2.158 | −0.596 | 34.559 | 1.00 | 12.54 |
| ATOM | 1423 | N | LEU | A | 713 | 2.156 | 0.275 | 36.625 | 1.00 | 12.50 |
| ATOM | 1424 | CA | LEU | A | 713 | 3.612 | 0.384 | 36.638 | 1.00 | 11.02 |
| ATOM | 1425 | CB | LEU | A | 713 | 4.105 | 1.023 | 37.940 | 1.00 | 13.58 |
| ATOM | 1426 | CG | LEU | A | 713 | 3.889 | 2.528 | 38.100 | 1.00 | 22.03 |
| ATOM | 1427 | CD1 | LEU | A | 713 | 4.754 | 3.042 | 39.250 | 1.00 | 21.64 |
| ATOM | 1428 | CD2 | LEU | A | 713 | 4.261 | 3.241 | 36.809 | 1.00 | 27.88 |
| ATOM | 1429 | C | LEU | A | 713 | 4.298 | −0.967 | 36.438 | 1.00 | 13.04 |
| ATOM | 1430 | O | LEU | A | 713 | 5.340 | −1.048 | 35.785 | 1.00 | 12.26 |
| ATOM | 1431 | N | LYS | A | 714 | 3.718 | −2.026 | 36.995 | 1.00 | 11.66 |
| ATOM | 1432 | CA | LYS | A | 714 | 4.304 | −3.356 | 36.852 | 1.00 | 12.24 |
| ATOM | 1433 | CB | LYS | A | 714 | 3.540 | −4.383 | 37.690 | 1.00 | 17.46 |
| ATOM | 1434 | CG | LYS | A | 714 | 3.582 | −4.123 | 39.182 | 1.00 | 27.51 |
| ATOM | 1435 | CD | LYS | A | 714 | 2.878 | −5.236 | 39.939 | 1.00 | 33.99 |
| ATOM | 1436 | CE | LYS | A | 714 | 2.899 | −4.988 | 41.433 | 1.00 | 42.93 |
| ATOM | 1437 | NZ | LYS | A | 714 | 2.222 | −6.092 | 42.168 | 1.00 | 46.33 |
| ATOM | 1438 | C | LYS | A | 714 | 4.284 | −3.784 | 35.390 | 1.00 | 12.70 |
| ATOM | 1439 | O | LYS | A | 714 | 5.259 | −4.338 | 34.886 | 1.00 | 12.91 |
| ATOM | 1440 | N | ILE | A | 715 | 3.168 | −3.525 | 34.717 | 1.00 | 10.95 |
| ATOM | 1441 | CA | ILE | A | 715 | 3.023 | −3.869 | 33.308 | 1.00 | 10.57 |
| ATOM | 1442 | CB | ILE | A | 715 | 1.586 | −3.590 | 32.820 | 1.00 | 11.39 |
| ATOM | 1443 | CG2 | ILE | A | 715 | 1.468 | −3.896 | 31.339 | 1.00 | 11.35 |
| ATOM | 1444 | CG1 | ILE | A | 715 | 0.596 | −4.450 | 33.613 | 1.00 | 12.69 |
| ATOM | 1445 | CD1 | ILE | A | 715 | −0.854 | −4.130 | 33.332 | 1.00 | 13.44 |
| ATOM | 1446 | C | ILE | A | 715 | 4.017 | −3.052 | 32.482 | 1.00 | 10.34 |
| ATOM | 1447 | O | ILE | A | 715 | 4.676 | −3.581 | 31.588 | 1.00 | 11.86 |
| ATOM | 1448 | N | ILE | A | 716 | 4.133 | −1.762 | 32.793 | 1.00 | 10.78 |
| ATOM | 1449 | CA | ILE | A | 716 | 5.069 | −0.886 | 32.081 | 1.00 | 9.90 |
| ATOM | 1450 | CB | ILE | A | 716 | 4.989 | 0.563 | 32.621 | 1.00 | 10.08 |
| ATOM | 1451 | CG2 | ILE | A | 716 | 6.171 | 1.387 | 32.115 | 1.00 | 10.86 |
| ATOM | 1452 | CG1 | ILE | A | 716 | 3.661 | 1.197 | 32.189 | 1.00 | 10.91 |
| ATOM | 1453 | CD1 | ILE | A | 716 | 3.413 | 2.564 | 32.792 | 1.00 | 11.27 |
| ATOM | 1454 | C | ILE | A | 716 | 6.508 | −1.393 | 32.196 | 1.00 | 8.77 |
| ATOM | 1455 | O | ILE | A | 716 | 7.229 | −1.487 | 31.199 | 1.00 | 9.34 |
| ATOM | 1456 | N | LYS | A | 717 | 6.926 | −1.724 | 33.413 | 1.00 | 9.18 |
| ATOM | 1457 | CA | LYS | A | 717 | 8.275 | −2.235 | 33.623 | 1.00 | 10.88 |
| ATOM | 1458 | CB | LYS | A | 717 | 8.489 | −2.606 | 35.087 | 1.00 | 12.47 |
| ATOM | 1459 | CG | LYS | A | 717 | 9.790 | −3.366 | 35.337 | 1.00 | 14.78 |
| ATOM | 1460 | CD | LYS | A | 717 | 9.865 | −3.864 | 36.775 | 1.00 | 17.70 |
| ATOM | 1461 | CE | LYS | A | 717 | 11.045 | −4.809 | 36.977 | 1.00 | 29.85 |
| ATOM | 1462 | NZ | LYS | A | 717 | 11.082 | −5.374 | 38.355 | 1.00 | 25.71 |
| ATOM | 1463 | C | LYS | A | 717 | 8.540 | −3.469 | 32.752 | 1.00 | 10.54 |
| ATOM | 1464 | O | LYS | A | 717 | 9.536 | −3.524 | 32.041 | 1.00 | 10.05 |
| ATOM | 1465 | N | GLN | A | 718 | 7.647 | −4.451 | 32.847 | 1.00 | 11.40 |
| ATOM | 1466 | CA | GLN | A | 718 | 7.774 | −5.682 | 32.067 | 1.00 | 11.80 |
| ATOM | 1467 | CB | GLN | A | 718 | 6.635 | −6.628 | 32.434 | 1.00 | 11.33 |
| ATOM | 1468 | CG | GLN | A | 718 | 6.683 | −6.965 | 33.899 | 1.00 | 14.75 |
| ATOM | 1469 | CD | GLN | A | 718 | 7.954 | −7.691 | 34.293 | 1.00 | 35.80 |
| ATOM | 1470 | OE1 | GLN | A | 718 | 8.774 | −7.176 | 35.053 | 1.00 | 49.66 |
| ATOM | 1471 | NE2 | GLN | A | 718 | 8.115 | −8.909 | 33.784 | 1.00 | 43.62 |
| ATOM | 1472 | C | GLN | A | 718 | 7.778 | −5.422 | 30.559 | 1.00 | 9.82 |
| ATOM | 1473 | O | GLN | A | 718 | 8.559 | −6.027 | 29.831 | 1.00 | 9.86 |
| ATOM | 1474 | N | ALA | A | 719 | 6.920 | −4.518 | 30.108 | 1.00 | 8.83 |
| ATOM | 1475 | CA | ALA | A | 719 | 6.842 | −4.205 | 28.686 | 1.00 | 10.49 |
| ATOM | 1476 | CB | ALA | A | 719 | 5.657 | −3.292 | 28.436 | 1.00 | 5.96 |
| ATOM | 1477 | C | ALA | A | 719 | 8.129 | −3.560 | 28.186 | 1.00 | 9.98 |
| ATOM | 1478 | O | ALA | A | 719 | 8.618 | −3.892 | 27.101 | 1.00 | 9.19 |
| ATOM | 1479 | N | ILE | A | 720 | 8.688 | −2.641 | 28.971 | 1.00 | 7.25 |
| ATOM | 1480 | CA | ILE | A | 720 | 9.920 | −1.988 | 28.548 | 1.00 | 8.66 |
| ATOM | 1481 | CB | ILE | A | 720 | 10.233 | −0.753 | 29.426 | 1.00 | 10.86 |
| ATOM | 1482 | CG2 | ILE | A | 720 | 11.616 | −0.193 | 29.086 | 1.00 | 11.64 |
| ATOM | 1483 | CG1 | ILE | A | 720 | 9.164 | 0.320 | 29.181 | 1.00 | 11.59 |
| ATOM | 1484 | CD1 | ILE | A | 720 | 9.367 | 1.605 | 29.976 | 1.00 | 12.21 |
| ATOM | 1485 | C | ILE | A | 720 | 11.085 | −2.974 | 28.580 | 1.00 | 10.02 |
| ATOM | 1486 | O | ILE | A | 720 | 11.883 | −3.040 | 27.641 | 1.00 | 8.95 |
| ATOM | 1487 | N | LEU | A | 721 | 11.178 | −3.762 | 29.643 | 1.00 | 8.10 |
| ATOM | 1488 | CA | LEU | A | 721 | 12.264 | −4.733 | 29.719 | 1.00 | 8.45 |
| ATOM | 1489 | CB | LEU | A | 721 | 12.251 | −5.446 | 31.077 | 1.00 | 10.55 |
| ATOM | 1490 | CG | LEU | A | 721 | 12.613 | −4.553 | 32.267 | 1.00 | 13.26 |
| ATOM | 1491 | CD1 | LEU | A | 721 | 12.539 | −5.350 | 33.574 | 1.00 | 15.78 |
| ATOM | 1492 | CD2 | LEU | A | 721 | 14.006 | −3.991 | 32.059 | 1.00 | 24.08 |
| ATOM | 1493 | C | LEU | A | 721 | 12.173 | −5.750 | 28.576 | 1.00 | 9.96 |
| ATOM | 1494 | O | LEU | A | 721 | 13.195 | −6.196 | 28.049 | 1.00 | 8.91 |
| ATOM | 1495 | N | ALA | A | 722 | 10.948 | −6.082 | 28.175 | 1.00 | 8.77 |
| ATOM | 1496 | CA | ALA | A | 722 | 10.715 | −7.047 | 27.100 | 1.00 | 8.74 |
| ATOM | 1497 | CB | ALA | A | 722 | 9.219 | −7.218 | 26.879 | 1.00 | 9.06 |
| ATOM | 1498 | C | ALA | A | 722 | 11.383 | −6.648 | 25.786 | 1.00 | 10.10 |
| ATOM | 1499 | O | ALA | A | 722 | 11.704 | −7.507 | 24.965 | 1.00 | 13.26 |
| ATOM | 1500 | N | THR | A | 723 | 11.598 | −5.350 | 25.585 | 1.00 | 8.97 |
| ATOM | 1501 | CA | THR | A | 723 | 12.213 | −4.884 | 24.344 | 1.00 | 10.94 |
| ATOM | 1502 | CB | THR | A | 723 | 11.943 | −3.374 | 24.127 | 1.00 | 8.53 |
| ATOM | 1503 | OG1 | THR | A | 723 | 12.542 | −2.615 | 25.181 | 1.00 | 8.41 |
| ATOM | 1504 | CG2 | THR | A | 723 | 10.437 | −3.114 | 24.118 | 1.00 | 8.22 |
| ATOM | 1505 | C | THR | A | 723 | 13.713 | −5.187 | 24.203 | 1.00 | 9.21 |
| ATOM | 1506 | O | THR | A | 723 | 14.327 | −4.862 | 23.186 | 1.00 | 12.03 |
| ATOM | 1507 | N | ASP | A | 724 | 14.308 | −5.797 | 25.223 | 1.00 | 9.75 |
| ATOM | 1508 | CA | ASP | A | 724 | 15.710 | −6.203 | 25.133 | 1.00 | 7.82 |
| ATOM | 1509 | CB | ASP | A | 724 | 16.257 | −6.533 | 26.525 | 1.00 | 12.18 |
| ATOM | 1510 | CG | ASP | A | 724 | 17.648 | −7.147 | 26.490 | 1.00 | 8.16 |
| ATOM | 1511 | OD1 | ASP | A | 724 | 18.232 | −7.299 | 25.394 | 1.00 | 11.70 |
| ATOM | 1512 | OD2 | ASP | A | 724 | 18.161 | −7.481 | 27.581 | 1.00 | 11.61 |
| ATOM | 1513 | C | ASP | A | 724 | 15.600 | −7.475 | 24.293 | 1.00 | 8.26 |
| ATOM | 1514 | O | ASP | A | 724 | 15.022 | −8.459 | 24.742 | 1.00 | 10.28 |
| ATOM | 1515 | N | LEU | A | 725 | 16.127 | −7.452 | 23.073 | 1.00 | 9.50 |
| ATOM | 1516 | CA | LEU | A | 725 | 16.037 | −8.611 | 22.189 | 1.00 | 8.83 |
| ATOM | 1517 | CB | LEU | A | 725 | 16.791 | −8.343 | 20.883 | 1.00 | 11.33 |
| ATOM | 1518 | CG | LEU | A | 725 | 16.365 | −9.236 | 19.717 | 1.00 | 16.17 |
| ATOM | 1519 | CD1 | LEU | A | 725 | 14.910 | −8.953 | 19.367 | 1.00 | 12.02 |
| ATOM | 1520 | CD2 | LEU | A | 725 | 17.262 | −8.974 | 18.516 | 1.00 | 20.09 |
| ATOM | 1521 | C | LEU | A | 725 | 16.573 | −9.880 | 22.849 | 1.00 | 12.30 |
| ATOM | 1522 | O | LEU | A | 725 | 16.179 | −10.994 | 22.498 | 1.00 | 11.52 |
| ATOM | 1523 | N | ALA | A | 726 | 17.476 | −9.712 | 23.806 | 1.00 | 11.08 |
| ATOM | 1524 | CA | ALA | A | 726 | 18.028 | −10.855 | 24.517 | 1.00 | 12.63 |
| ATOM | 1525 | CB | ALA | A | 726 | 19.104 | −10.394 | 25.489 | 1.00 | 21.41 |
| ATOM | 1526 | C | ALA | A | 726 | 16.918 | −11.583 | 25.274 | 1.00 | 12.40 |
| ATOM | 1527 | O | ALA | A | 726 | 16.914 | −12.813 | 25.354 | 1.00 | 13.56 |
| ATOM | 1528 | N | LEU | A | 727 | 15.985 | −10.823 | 25.843 | 1.00 | 12.79 |
| ATOM | 1529 | CA | LEU | A | 727 | 14.884 | −11.421 | 26.591 | 1.00 | 14.99 |
| ATOM | 1530 | CB | LEU | A | 727 | 14.206 | −10.380 | 27.485 | 1.00 | 11.71 |
| ATOM | 1531 | CG | LEU | A | 727 | 15.096 | −9.856 | 28.612 | 1.00 | 16.19 |
| ATOM | 1532 | CD1 | LEU | A | 727 | 14.302 | −8.925 | 29.511 | 1.00 | 13.74 |
| ATOM | 1533 | CD2 | LEU | A | 727 | 15.631 | −11.036 | 29.409 | 1.00 | 19.43 |
| ATOM | 1534 | C | LEU | A | 727 | 13.868 | −12.045 | 25.646 | 1.00 | 15.02 |
| ATOM | 1535 | O | LEU | A | 727 | 13.173 | −12.996 | 26.005 | 1.00 | 15.57 |
| ATOM | 1536 | N | TYR | A | 728 | 13.777 | −11.500 | 24.438 | 1.00 | 12.38 |
| ATOM | 1537 | CA | TYR | A | 728 | 12.862 | −12.038 | 23.435 | 1.00 | 12.71 |
| ATOM | 1538 | CB | TYR | A | 728 | 12.844 | −11.136 | 22.194 | 1.00 | 10.64 |
| ATOM | 1539 | CG | TYR | A | 728 | 12.303 | −11.823 | 20.960 | 1.00 | 12.57 |
| ATOM | 1540 | CD1 | TYR | A | 728 | 10.966 | −12.197 | 20.875 | 1.00 | 14.71 |
| ATOM | 1541 | CE1 | TYR | A | 728 | 10.473 | −12.854 | 19.752 | 1.00 | 12.42 |
| ATOM | 1542 | CD2 | TYR | A | 728 | 13.139 | −12.124 | 19.888 | 1.00 | 13.44 |
| ATOM | 1543 | CE2 | TYR | A | 728 | 12.659 | −12.777 | 18.763 | 1.00 | 13.13 |
| ATOM | 1544 | CZ | TYR | A | 728 | 11.329 | −13.142 | 18.699 | 1.00 | 15.27 |
| ATOM | 1545 | OH | TYR | A | 728 | 10.857 | −13.801 | 17.580 | 1.00 | 18.62 |
| ATOM | 1546 | C | TYR | A | 728 | 13.350 | −13.430 | 23.041 | 1.00 | 12.62 |
| ATOM | 1547 | O | TYR | A | 728 | 12.588 | −14.401 | 23.028 | 1.00 | 14.23 |
| ATOM | 1548 | N | ILE | A | 729 | 14.636 | −13.522 | 22.723 | 1.00 | 12.98 |
| ATOM | 1549 | CA | ILE | A | 729 | 15.232 | −14.788 | 22.327 | 1.00 | 13.19 |
| ATOM | 1550 | CB | ILE | A | 729 | 16.723 | −14.595 | 21.983 | 1.00 | 14.56 |
| ATOM | 1551 | CG2 | ILE | A | 729 | 17.395 | −15.942 | 21.797 | 1.00 | 19.39 |
| ATOM | 1552 | CG1 | ILE | A | 729 | 16.849 | −13.727 | 20.727 | 1.00 | 18.02 |
| ATOM | 1553 | CD1 | ILE | A | 729 | 18.268 | −13.309 | 20.406 | 1.00 | 26.38 |
| ATOM | 1554 | C | ILE | A | 729 | 15.088 | −15.826 | 23.439 | 1.00 | 15.70 |
| ATOM | 1555 | O | ILE | A | 729 | 14.830 | −17.001 | 23.178 | 1.00 | 14.36 |
| ATOM | 1556 | N | LYS | A | 730 | 15.227 | −15.375 | 24.681 | 1.00 | 15.21 |
| ATOM | 1557 | CA | LYS | A | 730 | 15.127 | −16.257 | 25.837 | 1.00 | 16.44 |
| ATOM | 1558 | CB | LYS | A | 730 | 15.657 | −15.528 | 27.075 | 1.00 | 21.43 |
| ATOM | 1559 | CG | LYS | A | 730 | 15.494 | −16.291 | 28.376 | 1.00 | 27.38 |
| ATOM | 1560 | CD | LYS | A | 730 | 16.133 | −15.528 | 29.523 | 1.00 | 31.22 |
| ATOM | 1561 | CE | LYS | A | 730 | 15.975 | −16.267 | 30.839 | 1.00 | 36.43 |
| ATOM | 1562 | NZ | LYS | A | 730 | 16.672 | −15.556 | 31.947 | 1.00 | 41.27 |
| ATOM | 1563 | C | LYS | A | 730 | 13.727 | −16.802 | 26.133 | 1.00 | 16.48 |
| ATOM | 1564 | O | LYS | A | 730 | 13.573 | −17.972 | 26.498 | 1.00 | 17.06 |
| ATOM | 1565 | N | ARG | A | 731 | 12.707 | −15.969 | 25.968 | 1.00 | 14.55 |
| ATOM | 1566 | CA | ARG | A | 731 | 11.347 | −16.395 | 26.283 | 1.00 | 15.57 |
| ATOM | 1567 | CB | ARG | A | 731 | 10.622 | −15.291 | 27.060 | 1.00 | 19.59 |
| ATOM | 1568 | CG | ARG | A | 731 | 11.309 | −14.842 | 28.335 | 1.00 | 18.25 |
| ATOM | 1569 | CD | ARG | A | 731 | 10.437 | −13.846 | 29.086 | 1.00 | 33.93 |
| ATOM | 1570 | NE | ARG | A | 731 | 9.209 | −14.462 | 29.588 | 1.00 | 32.78 |
| ATOM | 1571 | CZ | ARG | A | 731 | 8.168 | −13.781 | 30.058 | 1.00 | 29.98 |
| ATOM | 1572 | NH1 | ARG | A | 731 | 8.197 | −12.456 | 30.091 | 1.00 | 30.21 |
| ATOM | 1573 | NH2 | ARG | A | 731 | 7.097 | −14.426 | 30.501 | 1.00 | 36.09 |
| ATOM | 1574 | C | ARG | A | 731 | 10.461 | −16.807 | 25.118 | 1.00 | 12.75 |
| ATOM | 1575 | O | ARG | A | 731 | 9.436 | −17.454 | 25.325 | 1.00 | 15.30 |
| ATOM | 1576 | N | ARG | A | 732 | 10.841 | −16.445 | 23.897 | 1.00 | 13.21 |
| ATOM | 1577 | CA | ARG | A | 732 | 10.002 | −16.759 | 22.751 | 1.00 | 11.06 |
| ATOM | 1578 | CB | ARG | A | 732 | 10.589 | −16.162 | 21.474 | 1.00 | 10.97 |
| ATOM | 1579 | CG | ARG | A | 732 | 11.872 | −16.812 | 21.021 | 1.00 | 12.09 |
| ATOM | 1580 | CD | ARG | A | 732 | 12.325 | −16.192 | 19.724 | 1.00 | 12.79 |
| ATOM | 1581 | NE | ARG | A | 732 | 13.525 | −16.822 | 19.193 | 1.00 | 13.80 |
| ATOM | 1582 | CZ | ARG | A | 732 | 13.919 | −16.704 | 17.930 | 1.00 | 20.20 |
| ATOM | 1583 | NH1 | ARG | A | 732 | 13.201 | −15.980 | 17.077 | 1.00 | 23.11 |
| ATOM | 1584 | NH2 | ARG | A | 732 | 15.025 | −17.307 | 17.518 | 1.00 | 22.17 |
| ATOM | 1585 | C | ARG | A | 732 | 9.751 | −18.245 | 22.549 | 1.00 | 11.37 |
| ATOM | 1586 | O | ARG | A | 732 | 8.704 | −18.625 | 22.031 | 1.00 | 11.95 |
| ATOM | 1587 | N | GLY | A | 733 | 10.703 | −19.078 | 22.962 | 1.00 | 13.25 |
| ATOM | 1588 | CA | GLY | A | 733 | 10.543 | −20.512 | 22.804 | 1.00 | 13.40 |
| ATOM | 1589 | C | GLY | A | 733 | 9.298 | −21.009 | 23.508 | 1.00 | 14.10 |
| ATOM | 1590 | O | GLY | A | 733 | 8.543 | −21.823 | 22.970 | 1.00 | 15.18 |
| ATOM | 1591 | N | GLU | A | 734 | 9.080 | −20.512 | 24.719 | 1.00 | 11.89 |
| ATOM | 1592 | CA | GLU | A | 734 | 7.919 | −20.900 | 25.505 | 1.00 | 14.43 |
| ATOM | 1593 | CB | GLU | A | 734 | 7.988 | −20.264 | 26.895 | 1.00 | 14.05 |
| ATOM | 1594 | CG | GLU | A | 734 | 6.675 | −20.312 | 27.661 | 1.00 | 18.08 |
| ATOM | 1595 | CD | GLU | A | 734 | 6.810 | −19.828 | 29.091 | 1.00 | 21.97 |
| ATOM | 1596 | OE1 | GLU | A | 734 | 7.742 | −19.048 | 29.372 | 1.00 | 19.31 |
| ATOM | 1597 | OE2 | GLU | A | 734 | 5.977 | −20.221 | 29.932 | 1.00 | 32.96 |
| ATOM | 1598 | C | GLU | A | 734 | 6.632 | −20.478 | 24.806 | 1.00 | 14.65 |
| ATOM | 1599 | O | GLU | A | 734 | 5.663 | −21.235 | 24.760 | 1.00 | 14.67 |
| ATOM | 1600 | N | PHE | A | 735 | 6.631 | −19.264 | 24.267 | 1.00 | 15.01 |
| ATOM | 1601 | CA | PHE | A | 735 | 5.470 | −18.729 | 23.566 | 1.00 | 11.90 |
| ATOM | 1602 | CB | PHE | A | 735 | 5.743 | −17.278 | 23.157 | 1.00 | 11.17 |
| ATOM | 1603 | CG | PHE | A | 735 | 4.632 | −16.635 | 22.366 | 1.00 | 16.06 |
| ATOM | 1604 | CD1 | PHE | A | 735 | 3.354 | −16.515 | 22.892 | 1.00 | 16.32 |
| ATOM | 1605 | CD2 | PHE | A | 735 | 4.885 | −16.102 | 21.111 | 1.00 | 13.68 |
| ATOM | 1606 | CE1 | PHE | A | 735 | 2.355 | −15.869 | 22.178 | 1.00 | 13.80 |
| ATOM | 1607 | CE2 | PHE | A | 735 | 3.897 | −15.458 | 20.400 | 1.00 | 12.16 |
| ATOM | 1608 | CZ | PHE | A | 735 | 2.631 | −15.340 | 20.931 | 1.00 | 14.03 |
| ATOM | 1609 | C | PHE | A | 735 | 5.142 | −19.566 | 22.329 | 1.00 | 13.78 |
| ATOM | 1610 | O | PHE | A | 735 | 3.989 | −19.946 | 22.117 | 1.00 | 13.01 |
| ATOM | 1611 | N | PHE | A | 736 | 6.156 | −19.853 | 21.517 | 1.00 | 12.56 |
| ATOM | 1612 | CA | PHE | A | 736 | 5.952 | −20.632 | 20.299 | 1.00 | 14.09 |
| ATOM | 1613 | CB | PHE | A | 736 | 7.242 | −20.691 | 19.472 | 1.00 | 15.66 |
| ATOM | 1614 | CG | PHE | A | 736 | 7.752 | −19.342 | 19.031 | 1.00 | 17.77 |
| ATOM | 1615 | CD1 | PHE | A | 736 | 6.888 | −18.268 | 18.894 | 1.00 | 16.30 |
| ATOM | 1616 | CD2 | PHE | A | 736 | 9.092 | −19.166 | 18.701 | 1.00 | 14.18 |
| ATOM | 1617 | CE1 | PHE | A | 736 | 7.345 | −17.040 | 18.435 | 1.00 | 13.31 |
| ATOM | 1618 | CE2 | PHE | A | 736 | 9.558 | −17.939 | 18.242 | 1.00 | 13.12 |
| ATOM | 1619 | CZ | PHE | A | 736 | 8.686 | −16.877 | 18.106 | 1.00 | 17.60 |
| ATOM | 1620 | C | PHE | A | 736 | 5.483 | −22.054 | 20.598 | 1.00 | 14.02 |
| ATOM | 1621 | O | PHE | A | 736 | 4.653 | −22.608 | 19.874 | 1.00 | 15.27 |
| ATOM | 1622 | N | GLU | A | 737 | 6.022 | −22.640 | 21.661 | 1.00 | 15.53 |
| ATOM | 1623 | CA | GLU | A | 737 | 5.667 | −24.000 | 22.047 | 1.00 | 16.49 |
| ATOM | 1624 | CB | GLU | A | 737 | 6.598 | −24.493 | 23.155 | 1.00 | 18.16 |
| ATOM | 1625 | CG | GLU | A | 737 | 6.296 | −25.908 | 23.623 | 1.00 | 32.20 |
| ATOM | 1626 | CD | GLU | A | 737 | 7.266 | −26.397 | 24.685 | 1.00 | 44.41 |
| ATOM | 1627 | OE1 | GLU | A | 737 | 7.368 | −25.748 | 25.748 | 1.00 | 43.95 |
| ATOM | 1628 | OE2 | GLU | A | 737 | 7.926 | −27.433 | 24.457 | 1.00 | 45.00 |
| ATOM | 1629 | C | GLU | A | 737 | 4.219 | −24.105 | 22.505 | 1.00 | 16.86 |
| ATOM | 1630 | O | GLU | A | 737 | 3.517 | −25.050 | 22.137 | 1.00 | 18.64 |
| ATOM | 1631 | N | LEU | A | 738 | 3.774 | −23.144 | 23.308 | 1.00 | 15.93 |
| ATOM | 1632 | CA | LEU | A | 738 | 2.396 | −23.140 | 23.791 | 1.00 | 14.96 |
| ATOM | 1633 | CB | LEU | A | 738 | 2.137 | −21.910 | 24.665 | 1.00 | 13.51 |
| ATOM | 1634 | CG | LEU | A | 738 | 2.817 | −21.890 | 26.037 | 1.00 | 13.44 |
| ATOM | 1635 | CD1 | LEU | A | 738 | 2.813 | −20.474 | 26.589 | 1.00 | 14.08 |
| ATOM | 1636 | CD2 | LEU | A | 738 | 2.103 | −22.844 | 26.987 | 1.00 | 21.77 |
| ATOM | 1637 | C | LEU | A | 738 | 1.435 | −23.137 | 22.610 | 1.00 | 15.65 |
| ATOM | 1638 | O | LEU | A | 738 | 0.431 | −23.854 | 22.610 | 1.00 | 17.64 |
| ATOM | 1639 | N | ILE | A | 739 | 1.743 | −22.323 | 21.604 | 1.00 | 13.75 |
| ATOM | 1640 | CA | ILE | A | 739 | 0.904 | −22.236 | 20.415 | 1.00 | 14.28 |
| ATOM | 1641 | CB | ILE | A | 739 | 1.388 | −21.112 | 19.464 | 1.00 | 12.79 |
| ATOM | 1642 | CG2 | ILE | A | 739 | 0.686 | −21.224 | 18.118 | 1.00 | 23.84 |
| ATOM | 1643 | CG1 | ILE | A | 739 | 1.122 | −19.744 | 20.096 | 1.00 | 16.58 |
| ATOM | 1644 | CD1 | ILE | A | 739 | 1.656 | −18.576 | 19.279 | 1.00 | 13.81 |
| ATOM | 1645 | C | ILE | A | 739 | 0.925 | −23.557 | 19.654 | 1.00 | 16.17 |
| ATOM | 1646 | O | ILE | A | 739 | −0.126 | −24.107 | 19.309 | 1.00 | 18.24 |
| ATOM | 1647 | N | ARG | A | 740 | 2.124 | −24.075 | 19.410 | 1.00 | 15.35 |
| ATOM | 1648 | CA | ARG | A | 740 | 2.274 | −25.322 | 18.671 | 1.00 | 18.48 |
| ATOM | 1649 | CB | ARG | A | 740 | 3.758 | −25.648 | 18.474 | 1.00 | 23.15 |
| ATOM | 1650 | CG | ARG | A | 740 | 3.996 | −26.804 | 17.508 | 1.00 | 35.46 |
| ATOM | 1651 | CD | ARG | A | 740 | 5.475 | −27.059 | 17.224 | 1.00 | 39.66 |
| ATOM | 1652 | NE | ARG | A | 740 | 6.174 | −27.694 | 18.340 | 1.00 | 40.53 |
| ATOM | 1653 | CZ | ARG | A | 740 | 6.703 | −27.043 | 19.371 | 1.00 | 42.20 |
| ATOM | 1654 | NH1 | ARG | A | 740 | 6.623 | −25.721 | 19.441 | 1.00 | 52.88 |
| ATOM | 1655 | NH2 | ARG | A | 740 | 7.317 | −27.718 | 20.335 | 1.00 | 44.19 |
| ATOM | 1656 | C | ARG | A | 740 | 1.582 | −26.495 | 19.360 | 1.00 | 19.41 |
| ATOM | 1657 | O | ARG | A | 740 | 1.093 | −27.410 | 18.698 | 1.00 | 18.06 |
| ATOM | 1658 | N | LYS | A | 741 | 1.539 | −26.470 | 20.686 | 1.00 | 18.03 |
| ATOM | 1659 | CA | LYS | A | 741 | 0.906 | −27.553 | 21.430 | 1.00 | 18.11 |
| ATOM | 1660 | CB | LYS | A | 741 | 1.696 | −27.853 | 22.699 | 1.00 | 14.50 |
| ATOM | 1661 | CG | LYS | A | 741 | 3.074 | −28.426 | 22.451 | 1.00 | 21.67 |
| ATOM | 1662 | CD | LYS | A | 741 | 3.717 | −28.812 | 23.763 | 1.00 | 23.36 |
| ATOM | 1663 | CE | LYS | A | 741 | 5.087 | −29.430 | 23.558 | 1.00 | 27.38 |
| ATOM | 1664 | NZ | LYS | A | 741 | 5.715 | −29.747 | 24.871 | 1.00 | 23.91 |
| ATOM | 1665 | C | LYS | A | 741 | −0.540 | −27.248 | 21.798 | 1.00 | 17.80 |
| ATOM | 1666 | O | LYS | A | 741 | −1.134 | −27.940 | 22.623 | 1.00 | 18.63 |
| ATOM | 1667 | N | ASN | A | 742 | −1.102 | −26.214 | 21.183 | 1.00 | 17.40 |
| ATOM | 1668 | CA | ASN | A | 742 | −2.478 | −25.822 | 21.456 | 1.00 | 17.84 |
| ATOM | 1669 | CB | ASN | A | 742 | −3.443 | −26.841 | 20.849 | 1.00 | 16.88 |
| ATOM | 1670 | CG | ASN | A | 742 | −3.442 | −26.801 | 19.335 | 1.00 | 15.02 |
| ATOM | 1671 | OD1 | ASN | A | 742 | −3.741 | −25.772 | 18.733 | 1.00 | 28.97 |
| ATOM | 1672 | ND2 | ASN | A | 742 | −3.100 | −27.923 | 18.709 | 1.00 | 20.44 |
| ATOM | 1673 | C | ASN | A | 742 | −2.734 | −25.671 | 22.949 | 1.00 | 17.96 |
| ATOM | 1674 | O | ASN | A | 742 | −3.727 | −26.174 | 23.486 | 1.00 | 17.06 |
| ATOM | 1675 | N | GLN | A | 743 | −1.827 | −24.964 | 23.615 | 1.00 | 16.68 |
| ATOM | 1676 | CA | GLN | A | 743 | −1.937 | −24.727 | 25.045 | 1.00 | 17.92 |
| ATOM | 1677 | CB | GLN | A | 743 | −0.738 | −25.338 | 25.777 | 1.00 | 20.38 |
| ATOM | 1678 | CG | GLN | A | 743 | −0.461 | −26.787 | 25.408 | 1.00 | 28.60 |
| ATOM | 1679 | CD | GLN | A | 743 | 0.746 | −27.350 | 26.131 | 1.00 | 30.94 |
| ATOM | 1680 | OE1 | GLN | A | 743 | 1.775 | −26.682 | 26.255 | 1.00 | 32.52 |
| ATOM | 1681 | NE2 | GLN | A | 743 | 0.634 | −28.588 | 26.602 | 1.00 | 20.21 |
| ATOM | 1682 | C | GLN | A | 743 | −2.002 | −23.233 | 25.341 | 1.00 | 16.35 |
| ATOM | 1683 | O | GLN | A | 743 | −2.175 | −22.836 | 26.490 | 1.00 | 18.44 |
| ATOM | 1684 | N | PHE | A | 744 | −1.877 | −22.407 | 24.307 | 1.00 | 16.26 |
| ATOM | 1685 | CA | PHE | A | 744 | −1.916 | −20.959 | 24.501 | 1.00 | 19.79 |
| ATOM | 1686 | CB | PHE | A | 744 | −1.627 | −20.229 | 23.184 | 1.00 | 21.58 |
| ATOM | 1687 | CG | PHE | A | 744 | −1.465 | −18.740 | 23.340 | 1.00 | 17.97 |
| ATOM | 1688 | CD1 | PHE | A | 744 | −0.441 | −18.216 | 24.117 | 1.00 | 20.04 |
| ATOM | 1689 | CD2 | PHE | A | 744 | −2.345 | −17.865 | 22.720 | 1.00 | 24.29 |
| ATOM | 1690 | CE1 | PHE | A | 744 | −0.298 | −16.847 | 24.271 | 1.00 | 24.80 |
| ATOM | 1691 | CE2 | PHE | A | 744 | −2.208 | −16.499 | 22.869 | 1.00 | 20.38 |
| ATOM | 1692 | CZ | PHE | A | 744 | −1.184 | −15.988 | 23.647 | 1.00 | 26.24 |
| ATOM | 1693 | C | PHE | A | 744 | −3.265 | −20.515 | 25.060 | 1.00 | 19.74 |
| ATOM | 1694 | O | PHE | A | 744 | −4.316 | −20.860 | 24.526 | 1.00 | 21.03 |
| ATOM | 1695 | N | ASN | A | 745 | −3.221 | −19.746 | 26.142 | 1.00 | 19.11 |
| ATOM | 1696 | CA | ASN | A | 745 | −4.427 | −19.260 | 26.791 | 1.00 | 18.83 |
| ATOM | 1697 | CB | ASN | A | 745 | −4.893 | −20.270 | 27.839 | 1.00 | 14.11 |
| ATOM | 1698 | CG | ASN | A | 745 | −6.067 | −19.764 | 28.653 | 1.00 | 17.45 |
| ATOM | 1699 | OD1 | ASN | A | 745 | −6.924 | −19.042 | 28.143 | 1.00 | 22.19 |
| ATOM | 1700 | ND2 | ASN | A | 745 | −6.120 | −20.152 | 29.921 | 1.00 | 30.84 |
| ATOM | 1701 | C | ASN | A | 745 | −4.183 | −17.906 | 27.446 | 1.00 | 17.46 |
| ATOM | 1702 | O | ASN | A | 745 | −3.518 | −17.818 | 28.481 | 1.00 | 17.84 |
| ATOM | 1703 | N | LEU | A | 746 | −4.720 | −16.857 | 26.826 | 1.00 | 16.56 |
| ATOM | 1704 | CA | LEU | A | 746 | −4.573 | −15.497 | 27.323 | 1.00 | 18.48 |
| ATOM | 1705 | CB | LEU | A | 746 | −5.163 | −14.495 | 26.323 | 1.00 | 15.11 |
| ATOM | 1706 | CG | LEU | A | 746 | −4.319 | −14.192 | 25.082 | 1.00 | 17.98 |
| ATOM | 1707 | CD1 | LEU | A | 746 | −5.031 | −13.165 | 24.217 | 1.00 | 16.50 |
| ATOM | 1708 | CD2 | LEU | A | 746 | −2.960 | −13.664 | 25.507 | 1.00 | 17.71 |
| ATOM | 1709 | C | LEU | A | 746 | −5.212 | −15.284 | 28.688 | 1.00 | 21.08 |
| ATOM | 1710 | O | LEU | A | 746 | −4.911 | −14.304 | 29.366 | 1.00 | 20.60 |
| ATOM | 1711 | N | GLU | A | 747 | −6.098 | −16.192 | 29.089 | 1.00 | 21.14 |
| ATOM | 1712 | CA | GLU | A | 747 | −6.748 | −16.075 | 30.389 | 1.00 | 21.90 |
| ATOM | 1713 | CB | GLU | A | 747 | −7.916 | −17.058 | 30.504 | 1.00 | 20.33 |
| ATOM | 1714 | CG | GLU | A | 747 | −9.179 | −16.586 | 29.813 | 1.00 | 31.82 |
| ATOM | 1715 | CD | GLU | A | 747 | −10.324 | −17.569 | 29.959 | 1.00 | 40.12 |
| ATOM | 1716 | OE1 | GLU | A | 747 | −10.550 | −18.057 | 31.086 | 1.00 | 43.30 |
| ATOM | 1717 | OE2 | GLU | A | 747 | −11.003 | −17.846 | 28.949 | 1.00 | 40.94 |
| ATOM | 1718 | C | GLU | A | 747 | −5.740 | −16.331 | 31.498 | 1.00 | 22.98 |
| ATOM | 1719 | O | GLU | A | 747 | −5.885 | −15.826 | 32.612 | 1.00 | 23.46 |
| ATOM | 1720 | N | ASP | A | 748 | −4.719 | −17.124 | 31.188 | 1.00 | 21.44 |
| ATOM | 1721 | CA | ASP | A | 748 | −3.670 | −17.424 | 32.151 | 1.00 | 21.15 |
| ATOM | 1722 | CB | ASP | A | 748 | −2.800 | −18.579 | 31.651 | 1.00 | 19.25 |
| ATOM | 1723 | CG | ASP | A | 748 | −1.656 | −18.899 | 32.595 | 1.00 | 19.90 |
| ATOM | 1724 | OD1 | ASP | A | 748 | −0.710 | −18.088 | 32.692 | 1.00 | 26.86 |
| ATOM | 1725 | OD2 | ASP | A | 748 | −1.704 | −19.962 | 33.246 | 1.00 | 42.46 |
| ATOM | 1726 | C | ASP | A | 748 | −2.819 | −16.171 | 32.314 | 1.00 | 20.94 |
| ATOM | 1727 | O | ASP | A | 748 | −2.240 | −15.677 | 31.351 | 1.00 | 19.01 |
| ATOM | 1728 | N | PRO | A | 749 | −2.742 | −15.637 | 33.541 | 1.00 | 18.53 |
| ATOM | 1729 | CD | PRO | A | 749 | −3.443 | −16.108 | 34.750 | 1.00 | 26.08 |
| ATOM | 1730 | CA | PRO | A | 749 | −1.958 | −14.432 | 33.825 | 1.00 | 19.83 |
| ATOM | 1731 | CB | PRO | A | 749 | −2.009 | −14.346 | 35.347 | 1.00 | 25.90 |
| ATOM | 1732 | CG | PRO | A | 749 | −3.377 | −14.896 | 35.654 | 1.00 | 25.60 |
| ATOM | 1733 | C | PRO | A | 749 | −0.528 | −14.476 | 33.286 | 1.00 | 17.96 |
| ATOM | 1734 | O | PRO | A | 749 | −0.047 | −13.506 | 32.708 | 1.00 | 18.09 |
| ATOM | 1735 | N | HIS | A | 750 | 0.149 | −15.603 | 33.475 | 1.00 | 17.27 |
| ATOM | 1736 | CA | HIS | A | 750 | 1.520 | −15.737 | 32.998 | 1.00 | 16.70 |
| ATOM | 1737 | CB | HIS | A | 750 | 2.138 | −17.039 | 33.509 | 1.00 | 14.57 |
| ATOM | 1738 | CG | HIS | A | 750 | 3.506 | −17.301 | 32.975 | 1.00 | 13.17 |
| ATOM | 1739 | CD2 | HIS | A | 750 | 4.710 | −16.780 | 33.299 | 1.00 | 15.22 |
| ATOM | 1740 | ND1 | HIS | A | 750 | 3.748 | −18.181 | 31.933 | 1.00 | 20.55 |
| ATOM | 1741 | CE1 | HIS | A | 750 | 5.030 | −18.183 | 31.649 | 1.00 | 21.44 |
| ATOM | 1742 | NE2 | HIS | A | 750 | 5.644 | −17.338 | 32.465 | 1.00 | 27.33 |
| ATOM | 1743 | C | HIS | A | 750 | 1.615 | −15.691 | 31.475 | 1.00 | 16.04 |
| ATOM | 1744 | O | HIS | A | 750 | 2.521 | −15.069 | 30.925 | 1.00 | 16.50 |
| ATOM | 1745 | N | GLU | A | 751 | 0.680 | −16.346 | 30.794 | 1.00 | 14.76 |
| ATOM | 1746 | CA | GLU | A | 751 | 0.689 | −16.359 | 29.333 | 1.00 | 14.97 |
| ATOM | 1747 | CB | GLU | A | 751 | −0.269 | −17.427 | 28.798 | 1.00 | 11.89 |
| ATOM | 1748 | CG | GLU | A | 751 | 0.173 | −18.848 | 29.123 | 1.00 | 16.94 |
| ATOM | 1749 | CD | GLU | A | 751 | −0.601 | −19.892 | 28.354 | 1.00 | 17.06 |
| ATOM | 1750 | OE1 | GLU | A | 751 | −0.804 | −19.697 | 27.140 | 1.00 | 17.15 |
| ATOM | 1751 | OE2 | GLU | A | 751 | −0.993 | −20.912 | 28.959 | 1.00 | 27.83 |
| ATOM | 1752 | C | GLU | A | 751 | 0.325 | −14.988 | 28.764 | 1.00 | 14.11 |
| ATOM | 1753 | O | GLU | A | 751 | 0.782 | −14.616 | 27.685 | 1.00 | 14.18 |
| ATOM | 1754 | N | LYS | A | 752 | −0.501 | −14.248 | 29.498 | 1.00 | 13.06 |
| ATOM | 1755 | CA | LYS | A | 752 | −0.900 | −12.910 | 29.092 | 1.00 | 11.63 |
| ATOM | 1756 | CB | LYS | A | 752 | −1.984 | −12.370 | 30.041 | 1.00 | 11.74 |
| ATOM | 1757 | CG | LYS | A | 752 | −2.200 | −10.865 | 29.981 | 1.00 | 11.33 |
| ATOM | 1758 | CD | LYS | A | 752 | −2.667 | −10.402 | 28.608 | 1.00 | 18.51 |
| ATOM | 1759 | CE | LYS | A | 752 | −3.921 | −11.138 | 28.184 | 1.00 | 31.49 |
| ATOM | 1760 | NZ | LYS | A | 752 | −4.536 | −10.523 | 26.978 | 1.00 | 47.29 |
| ATOM | 1761 | C | LYS | A | 752 | 0.362 | −12.047 | 29.176 | 1.00 | 14.58 |
| ATOM | 1762 | O | LYS | A | 752 | 0.665 | −11.293 | 28.249 | 1.00 | 11.11 |
| ATOM | 1763 | N | GLU | A | 753 | 1.093 | −12.196 | 30.270 | 1.00 | 12.46 |
| ATOM | 1764 | CA | GLU | A | 753 | 2.315 | −11.409 | 30.445 | 1.00 | 13.89 |
| ATOM | 1765 | CB | GLU | A | 753 | 2.939 | −11.745 | 31.801 | 1.00 | 15.41 |
| ATOM | 1766 | CG | GLU | A | 753 | 4.187 | −10.950 | 32.098 | 1.00 | 17.51 |
| ATOM | 1767 | CD | GLU | A | 753 | 4.946 | −11.478 | 33.300 | 1.00 | 36.21 |
| ATOM | 1768 | OE1 | GLU | A | 753 | 4.335 | −11.579 | 34.384 | 1.00 | 28.23 |
| ATOM | 1769 | OE2 | GLU | A | 753 | 6.150 | −11.782 | 33.162 | 1.00 | 36.81 |
| ATOM | 1770 | C | GLU | A | 753 | 3.267 | −11.729 | 29.313 | 1.00 | 14.45 |
| ATOM | 1771 | O | GLU | A | 753 | 3.852 | −10.839 | 28.671 | 1.00 | 11.34 |
| ATOM | 1772 | N | LEU | A | 754 | 3.441 | −13.018 | 29.035 | 1.00 | 12.90 |
| ATOM | 1773 | CA | LEU | A | 754 | 4.329 | −13.471 | 27.974 | 1.00 | 13.51 |
| ATOM | 1774 | CB | LEU | A | 754 | 4.377 | −15.011 | 27.908 | 1.00 | 13.76 |
| ATOM | 1775 | CG | LEU | A | 754 | 5.196 | −15.682 | 26.793 | 1.00 | 15.32 |
| ATOM | 1776 | CD1 | LEU | A | 754 | 6.663 | −15.286 | 26.877 | 1.00 | 15.56 |
| ATOM | 1777 | CD2 | LEU | A | 754 | 5.044 | −17.193 | 26.930 | 1.00 | 14.90 |
| ATOM | 1778 | C | LEU | A | 754 | 3.915 | −12.900 | 26.616 | 1.00 | 12.21 |
| ATOM | 1779 | O | LEU | A | 754 | 4.765 | −12.478 | 25.821 | 1.00 | 11.37 |
| ATOM | 1780 | N | PHE | A | 755 | 2.613 | −12.867 | 26.351 | 1.00 | 11.00 |
| ATOM | 1781 | CA | PHE | A | 755 | 2.119 | −12.336 | 25.088 | 1.00 | 9.92 |
| ATOM | 1782 | CB | PHE | A | 755 | 0.609 | −12.551 | 24.966 | 1.00 | 12.44 |
| ATOM | 1783 | CG | PHE | A | 755 | 0.002 | −11.867 | 23.780 | 1.00 | 11.67 |
| ATOM | 1784 | CD1 | PHE | A | 755 | 0.338 | −12.259 | 22.492 | 1.00 | 13.36 |
| ATOM | 1785 | CD2 | PHE | A | 755 | −0.858 | −10.792 | 23.948 | 1.00 | 9.93 |
| ATOM | 1786 | CE1 | PHE | A | 755 | −0.170 | −11.590 | 21.393 | 1.00 | 14.56 |
| ATOM | 1787 | CE2 | PHE | A | 755 | −1.369 | −10.118 | 22.854 | 1.00 | 13.93 |
| ATOM | 1788 | CZ | PHE | A | 755 | −1.021 | −10.517 | 21.574 | 1.00 | 18.65 |
| ATOM | 1789 | C | PHE | A | 755 | 2.431 | −10.840 | 24.972 | 1.00 | 9.95 |
| ATOM | 1790 | O | PHE | A | 755 | 2.856 | −10.367 | 23.916 | 1.00 | 10.28 |
| ATOM | 1791 | N | LEU | A | 756 | 2.225 | −10.099 | 26.057 | 1.00 | 9.98 |
| ATOM | 1792 | CA | LEU | A | 756 | 2.489 | −8.664 | 26.034 | 1.00 | 9.03 |
| ATOM | 1793 | CB | LEU | A | 756 | 2.102 | −8.017 | 27.370 | 1.00 | 9.14 |
| ATOM | 1794 | CG | LEU | A | 756 | 0.610 | −8.119 | 27.706 | 1.00 | 13.27 |
| ATOM | 1795 | CD1 | LEU | A | 756 | 0.331 | −7.487 | 29.065 | 1.00 | 21.21 |
| ATOM | 1796 | CD2 | LEU | A | 756 | −0.198 | −7.432 | 26.613 | 1.00 | 18.57 |
| ATOM | 1797 | C | LEU | A | 756 | 3.959 | −8.410 | 25.721 | 1.00 | 9.36 |
| ATOM | 1798 | O | LEU | A | 756 | 4.291 | −7.437 | 25.045 | 1.00 | 8.42 |
| ATOM | 1799 | N | ALA | A | 757 | 4.844 | −9.285 | 26.197 | 1.00 | 7.41 |
| ATOM | 1800 | CA | ALA | A | 757 | 6.267 | −9.118 | 25.915 | 1.00 | 10.17 |
| ATOM | 1801 | CB | ALA | A | 757 | 7.096 | −10.112 | 26.720 | 1.00 | 10.57 |
| ATOM | 1802 | C | ALA | A | 757 | 6.514 | −9.315 | 24.421 | 1.00 | 9.99 |
| ATOM | 1803 | O | ALA | A | 757 | 7.254 | −8.548 | 23.798 | 1.00 | 10.22 |
| ATOM | 1804 | N | MET | A | 758 | 5.890 | −10.341 | 23.845 | 1.00 | 8.41 |
| ATOM | 1805 | CA | MET | A | 758 | 6.046 | −10.627 | 22.422 | 1.00 | 8.55 |
| ATOM | 1806 | CB | MET | A | 758 | 5.348 | −11.946 | 22.061 | 1.00 | 8.57 |
| ATOM | 1807 | CG | MET | A | 758 | 5.898 | −13.175 | 22.783 | 1.00 | 9.97 |
| ATOM | 1808 | SD | MET | A | 758 | 7.613 | −13.597 | 22.367 | 1.00 | 15.99 |
| ATOM | 1809 | CE | MET | A | 758 | 7.436 | −14.226 | 20.702 | 1.00 | 36.66 |
| ATOM | 1810 | C | MET | A | 758 | 5.469 | −9.499 | 21.572 | 1.00 | 9.31 |
| ATOM | 1811 | O | MET | A | 758 | 6.035 | −9.134 | 20.534 | 1.00 | 8.67 |
| ATOM | 1812 | N | LEU | A | 759 | 4.334 | −8.962 | 22.008 | 1.00 | 10.68 |
| ATOM | 1813 | CA | LEU | A | 759 | 3.682 | −7.877 | 21.290 | 1.00 | 7.26 |
| ATOM | 1814 | CB | LEU | A | 759 | 2.348 | −7.536 | 21.954 | 1.00 | 6.70 |
| ATOM | 1815 | CG | LEU | A | 759 | 1.521 | −6.419 | 21.316 | 1.00 | 9.88 |
| ATOM | 1816 | CD1 | LEU | A | 759 | 1.217 | −6.751 | 19.858 | 1.00 | 13.46 |
| ATOM | 1817 | CD2 | LEU | A | 759 | 0.232 | −6.241 | 22.103 | 1.00 | 8.91 |
| ATOM | 1818 | C | LEU | A | 759 | 4.589 | −6.650 | 21.269 | 1.00 | 10.65 |
| ATOM | 1819 | O | LEU | A | 759 | 4.677 | −5.945 | 20.259 | 1.00 | 8.66 |
| ATOM | 1820 | N | MET | A | 760 | 5.258 | −6.388 | 22.387 | 1.00 | 8.87 |
| ATOM | 1821 | CA | MET | A | 760 | 6.168 | −5.251 | 22.454 | 1.00 | 9.10 |
| ATOM | 1822 | CB | MET | A | 760 | 6.760 | −5.114 | 23.859 | 1.00 | 9.37 |
| ATOM | 1823 | CG | MET | A | 760 | 5.831 | −4.491 | 24.889 | 1.00 | 7.94 |
| ATOM | 1824 | SD | MET | A | 760 | 5.392 | −2.762 | 24.513 | 1.00 | 11.15 |
| ATOM | 1825 | CE | MET | A | 760 | 6.981 | −1.951 | 24.748 | 1.00 | 10.58 |
| ATOM | 1826 | C | MET | A | 760 | 7.292 | −5.445 | 21.444 | 1.00 | 9.36 |
| ATOM | 1827 | O | MET | A | 760 | 7.693 | −4.501 | 20.757 | 1.00 | 8.44 |
| ATOM | 1828 | N | THR | A | 761 | 7.812 | −6.667 | 21.359 | 1.00 | 8.62 |
| ATOM | 1829 | CA | THR | A | 761 | 8.890 | −6.948 | 20.416 | 1.00 | 9.46 |
| ATOM | 1830 | CB | THR | A | 761 | 9.442 | −8.379 | 20.597 | 1.00 | 12.42 |
| ATOM | 1831 | OG1 | THR | A | 761 | 9.916 | −8.540 | 21.940 | 1.00 | 11.86 |
| ATOM | 1832 | CG2 | THR | A | 761 | 10.598 | −8.636 | 19.620 | 1.00 | 9.90 |
| ATOM | 1833 | C | THR | A | 761 | 8.389 | −6.785 | 18.984 | 1.00 | 10.31 |
| ATOM | 1834 | O | THR | A | 761 | 9.088 | −6.238 | 18.128 | 1.00 | 10.55 |
| ATOM | 1835 | N | ALA | A | 762 | 7.172 | −7.254 | 18.722 | 1.00 | 10.42 |
| ATOM | 1836 | CA | ALA | A | 762 | 6.598 | −7.148 | 17.389 | 1.00 | 7.73 |
| ATOM | 1837 | CB | ALA | A | 762 | 5.202 | −7.777 | 17.362 | 1.00 | 10.79 |
| ATOM | 1838 | C | ALA | A | 762 | 6.524 | −5.689 | 16.948 | 1.00 | 9.57 |
| ATOM | 1839 | O | ALA | A | 762 | 6.794 | −5.370 | 15.787 | 1.00 | 9.59 |
| ATOM | 1840 | N | CYS | A | 763 | 6.161 | −4.801 | 17.872 | 1.00 | 7.53 |
| ATOM | 1841 | CA | CYS | A | 763 | 6.063 | −3.378 | 17.551 | 1.00 | 7.89 |
| ATOM | 1842 | CB | CYS | A | 763 | 5.237 | −2.641 | 18.611 | 1.00 | 11.70 |
| ATOM | 1843 | SG | CYS | A | 763 | 3.503 | −3.143 | 18.670 | 1.00 | 11.19 |
| ATOM | 1844 | C | CYS | A | 763 | 7.441 | −2.744 | 17.443 | 1.00 | 9.03 |
| ATOM | 1845 | O | CYS | A | 763 | 7.689 | −1.911 | 16.574 | 1.00 | 10.26 |
| ATOM | 1846 | N | ASP | A | 764 | 8.340 | −3.157 | 18.329 | 1.00 | 8.73 |
| ATOM | 1847 | CA | ASP | A | 764 | 9.703 | −2.641 | 18.352 | 1.00 | 10.03 |
| ATOM | 1848 | CB | ASP | A | 764 | 10.448 | −3.269 | 19.541 | 1.00 | 11.85 |
| ATOM | 1849 | CG | ASP | A | 764 | 11.766 | −2.585 | 19.840 | 1.00 | 12.03 |
| ATOM | 1850 | OD1 | ASP | A | 764 | 11.963 | −1.451 | 19.361 | 1.00 | 11.46 |
| ATOM | 1851 | OD2 | ASP | A | 764 | 12.592 | −3.176 | 20.571 | 1.00 | 15.03 |
| ATOM | 1852 | C | ASP | A | 764 | 10.447 | −2.924 | 17.036 | 1.00 | 10.20 |
| ATOM | 1853 | O | ASP | A | 764 | 11.225 | −2.097 | 16.562 | 1.00 | 11.71 |
| ATOM | 1854 | N | LEU | A | 765 | 10.194 | −4.086 | 16.442 | 1.00 | 11.54 |
| ATOM | 1855 | CA | LEU | A | 765 | 10.867 | −4.474 | 15.202 | 1.00 | 11.71 |
| ATOM | 1856 | CB | LEU | A | 765 | 11.208 | −5.965 | 15.252 | 1.00 | 13.42 |
| ATOM | 1857 | CG | LEU | A | 765 | 12.017 | −6.454 | 16.453 | 1.00 | 15.02 |
| ATOM | 1858 | CD1 | LEU | A | 765 | 12.233 | −7.957 | 16.348 | 1.00 | 13.91 |
| ATOM | 1859 | CD2 | LEU | A | 765 | 13.345 | −5.721 | 16.499 | 1.00 | 16.31 |
| ATOM | 1860 | C | LEU | A | 765 | 10.049 | −4.221 | 13.941 | 1.00 | 12.10 |
| ATOM | 1861 | O | LEU | A | 765 | 10.490 | −4.560 | 12.837 | 1.00 | 11.30 |
| ATOM | 1862 | N | SER | A | 766 | 8.881 | −3.606 | 14.101 | 1.00 | 12.76 |
| ATOM | 1863 | CA | SER | A | 766 | 7.965 | −3.383 | 12.983 | 1.00 | 10.86 |
| ATOM | 1864 | CB | SER | A | 766 | 6.672 | −2.733 | 13.488 | 1.00 | 8.49 |
| ATOM | 1865 | OG | SER | A | 766 | 6.902 | −1.438 | 13.991 | 1.00 | 14.83 |
| ATOM | 1866 | C | SER | A | 766 | 8.446 | −2.657 | 11.729 | 1.00 | 7.60 |
| ATOM | 1867 | O | SER | A | 766 | 7.782 | −2.735 | 10.700 | 1.00 | 9.56 |
| ATOM | 1868 | N | ALA | A | 767 | 9.575 | −1.956 | 11.787 | 1.00 | 8.02 |
| ATOM | 1869 | CA | ALA | A | 767 | 10.073 | −1.296 | 10.581 | 1.00 | 9.45 |
| ATOM | 1870 | CB | ALA | A | 767 | 11.364 | −0.522 | 10.881 | 1.00 | 11.41 |
| ATOM | 1871 | C | ALA | A | 767 | 10.340 | −2.377 | 9.521 | 1.00 | 10.04 |
| ATOM | 1872 | O | ALA | A | 767 | 10.253 | −2.124 | 8.319 | 1.00 | 10.04 |
| ATOM | 1873 | N | ILE | A | 768 | 10.655 | −3.586 | 9.976 | 1.00 | 9.49 |
| ATOM | 1874 | CA | ILE | A | 768 | 10.942 | −4.694 | 9.066 | 1.00 | 12.99 |
| ATOM | 1875 | CB | ILE | A | 768 | 11.555 | −5.894 | 9.840 | 1.00 | 9.64 |
| ATOM | 1876 | CG2 | ILE | A | 768 | 10.473 | −6.631 | 10.612 | 1.00 | 7.91 |
| ATOM | 1877 | CG1 | ILE | A | 768 | 12.270 | −6.836 | 8.868 | 1.00 | 12.83 |
| ATOM | 1878 | CD1 | ILE | A | 768 | 13.471 | −6.199 | 8.190 | 1.00 | 15.69 |
| ATOM | 1879 | C | ILE | A | 768 | 9.704 | −5.173 | 8.296 | 1.00 | 11.57 |
| ATOM | 1880 | O | ILE | A | 768 | 9.812 | −5.986 | 7.376 | 1.00 | 12.45 |
| ATOM | 1881 | N | THR | A | 769 | 8.530 | −4.665 | 8.662 | 1.00 | 12.70 |
| ATOM | 1882 | CA | THR | A | 769 | 7.286 | −5.062 | 7.999 | 1.00 | 10.88 |
| ATOM | 1883 | CB | THR | A | 769 | 6.167 | −5.326 | 9.025 | 1.00 | 13.25 |
| ATOM | 1884 | OG1 | THR | A | 769 | 5.760 | −4.083 | 9.617 | 1.00 | 12.15 |
| ATOM | 1885 | CG2 | THR | A | 769 | 6.654 | −6.271 | 10.124 | 1.00 | 16.86 |
| ATOM | 1886 | C | THR | A | 769 | 6.762 | −3.991 | 7.036 | 1.00 | 12.18 |
| ATOM | 1887 | O | THR | A | 769 | 5.795 | −4.223 | 6.308 | 1.00 | 13.18 |
| ATOM | 1888 | N | LYS | A | 770 | 7.402 | −2.824 | 7.043 | 1.00 | 12.64 |
| ATOM | 1889 | CA | LYS | A | 770 | 6.986 | −1.686 | 6.216 | 1.00 | 10.43 |
| ATOM | 1890 | CB | LYS | A | 770 | 7.684 | −0.413 | 6.714 | 1.00 | 12.22 |
| ATOM | 1891 | CG | LYS | A | 770 | 7.375 | −0.029 | 8.165 | 1.00 | 7.87 |
| ATOM | 1892 | CD | LYS | A | 770 | 5.948 | 0.457 | 8.330 | 1.00 | 13.33 |
| ATOM | 1893 | CE | LYS | A | 770 | 5.644 | 0.766 | 9.794 | 1.00 | 14.40 |
| ATOM | 1894 | NZ | LYS | A | 770 | 4.263 | 1.293 | 9.973 | 1.00 | 14.45 |
| ATOM | 1895 | C | LYS | A | 770 | 7.247 | −1.829 | 4.716 | 1.00 | 11.91 |
| ATOM | 1896 | O | LYS | A | 770 | 8.069 | −2.638 | 4.290 | 1.00 | 13.63 |
| ATOM | 1897 | N | PRO | A | 771 | 6.529 | −1.041 | 3.892 | 1.00 | 11.52 |
| ATOM | 1898 | CD | PRO | A | 771 | 5.408 | −0.150 | 4.240 | 1.00 | 14.60 |
| ATOM | 1899 | CA | PRO | A | 771 | 6.718 | −1.096 | 2.441 | 1.00 | 12.80 |
| ATOM | 1900 | CB | PRO | A | 771 | 5.914 | 0.099 | 1.950 | 1.00 | 15.88 |
| ATOM | 1901 | CG | PRO | A | 771 | 4.751 | 0.098 | 2.886 | 1.00 | 11.15 |
| ATOM | 1902 | C | PRO | A | 771 | 8.206 | −0.933 | 2.183 | 1.00 | 14.66 |
| ATOM | 1903 | O | PRO | A | 771 | 8.880 | −0.201 | 2.908 | 1.00 | 15.01 |
| ATOM | 1904 | N | TRP | A | 772 | 8.711 | −1.604 | 1.156 | 1.00 | 14.26 |
| ATOM | 1905 | CA | TRP | A | 772 | 10.133 | −1.555 | 0.827 | 1.00 | 15.34 |
| ATOM | 1906 | CB | TRP | A | 772 | 10.367 | −2.121 | −0.578 | 1.00 | 14.10 |
| ATOM | 1907 | CG | TRP | A | 772 | 11.809 | −2.101 | −1.009 | 1.00 | 17.47 |
| ATOM | 1908 | CD2 | TRP | A | 772 | 12.931 | −2.659 | −0.307 | 1.00 | 17.61 |
| ATOM | 1909 | CE2 | TRP | A | 772 | 14.079 | −2.404 | −1.086 | 1.00 | 19.63 |
| ATOM | 1910 | CE3 | TRP | A | 772 | 13.076 | −3.348 | 0.901 | 1.00 | 16.69 |
| ATOM | 1911 | CD1 | TRP | A | 772 | 12.313 | −1.547 | −2.152 | 1.00 | 16.69 |
| ATOM | 1912 | NE1 | TRP | A | 772 | 13.675 | −1.725 | −2.204 | 1.00 | 24.41 |
| ATOM | 1913 | CZ2 | TRP | A | 772 | 15.354 | −2.816 | −0.694 | 1.00 | 24.29 |
| ATOM | 1914 | CZ3 | TRP | A | 772 | 14.344 | −3.755 | 1.289 | 1.00 | 17.48 |
| ATOM | 1915 | CH2 | TRP | A | 772 | 15.466 | −3.488 | 0.493 | 1.00 | 21.25 |
| ATOM | 1916 | C | TRP | A | 772 | 10.820 | −0.190 | 0.952 | 1.00 | 17.78 |
| ATOM | 1917 | O | TRP | A | 772 | 11.834 | −0.070 | 1.639 | 1.00 | 15.81 |
| ATOM | 1918 | N | PRO | A | 773 | 10.286 | 0.853 | 0.291 | 1.00 | 19.35 |
| ATOM | 1919 | CD | PRO | A | 773 | 9.132 | 0.877 | −0.624 | 1.00 | 18.85 |
| ATOM | 1920 | CA | PRO | A | 773 | 10.909 | 2.180 | 0.377 | 1.00 | 18.24 |
| ATOM | 1921 | CB | PRO | A | 773 | 9.926 | 3.068 | −0.382 | 1.00 | 21.17 |
| ATOM | 1922 | CG | PRO | A | 773 | 9.386 | 2.138 | −1.422 | 1.00 | 23.60 |
| ATOM | 1923 | C | PRO | A | 773 | 11.129 | 2.654 | 1.814 | 1.00 | 16.33 |
| ATOM | 1924 | O | PRO | A | 773 | 12.167 | 3.235 | 2.140 | 1.00 | 16.01 |
| ATOM | 1925 | N | ILE | A | 774 | 10.143 | 2.406 | 2.668 | 1.00 | 16.23 |
| ATOM | 1926 | CA | ILE | A | 774 | 10.228 | 2.805 | 4.066 | 1.00 | 15.30 |
| ATOM | 1927 | CB | ILE | A | 774 | 8.851 | 2.670 | 4.751 | 1.00 | 15.99 |
| ATOM | 1928 | CG2 | ILE | A | 774 | 8.977 | 2.922 | 6.250 | 1.00 | 13.24 |
| ATOM | 1929 | CG1 | ILE | A | 774 | 7.867 | 3.658 | 4.110 | 1.00 | 20.68 |
| ATOM | 1930 | CD1 | ILE | A | 774 | 6.441 | 3.501 | 4.588 | 1.00 | 21.74 |
| ATOM | 1931 | C | ILE | A | 774 | 11.264 | 1.958 | 4.804 | 1.00 | 16.31 |
| ATOM | 1932 | O | ILE | A | 774 | 12.133 | 2.491 | 5.497 | 1.00 | 15.63 |
| ATOM | 1933 | N | GLN | A | 775 | 11.173 | 0.640 | 4.650 | 1.00 | 13.80 |
| ATOM | 1934 | CA | GLN | A | 775 | 12.120 | −0.274 | 5.279 | 1.00 | 12.85 |
| ATOM | 1935 | CB | GLN | A | 775 | 11.786 | −1.727 | 4.901 | 1.00 | 13.34 |
| ATOM | 1936 | CG | GLN | A | 775 | 12.987 | −2.688 | 4.921 | 1.00 | 14.20 |
| ATOM | 1937 | CD | GLN | A | 775 | 13.582 | −2.897 | 6.305 | 1.00 | 21.70 |
| ATOM | 1938 | OE1 | GLN | A | 775 | 14.663 | −3.472 | 6.441 | 1.00 | 13.81 |
| ATOM | 1939 | NE2 | GLN | A | 775 | 12.880 | −2.440 | 7.336 | 1.00 | 14.50 |
| ATOM | 1940 | C | GLN | A | 775 | 13.549 | 0.057 | 4.877 | 1.00 | 13.13 |
| ATOM | 1941 | O | GLN | A | 775 | 14.458 | 0.060 | 5.694 | 1.00 | 12.67 |
| ATOM | 1942 | N | GLN | A | 776 | 13.749 | 0.352 | 3.590 | 1.00 | 10.04 |
| ATOM | 1943 | CA | GLN | A | 776 | 15.068 | 0.674 | 3.090 | 1.00 | 12.84 |
| ATOM | 1944 | CB | GLN | A | 776 | 15.004 | 0.820 | 1.564 | 1.00 | 19.00 |
| ATOM | 1945 | CG | GLN | A | 776 | 16.326 | 0.943 | 0.890 | 1.00 | 25.33 |
| ATOM | 1946 | CD | GLN | A | 776 | 16.172 | 0.760 | −0.595 | 1.00 | 36.37 |
| ATOM | 1947 | OE1 | GLN | A | 776 | 15.258 | 1.315 | −1.209 | 1.00 | 39.80 |
| ATOM | 1948 | NE2 | GLN | A | 776 | 17.058 | −0.023 | −1.186 | 1.00 | 41.16 |
| ATOM | 1949 | C | GLN | A | 776 | 15.627 | 1.934 | 3.738 | 1.00 | 10.61 |
| ATOM | 1950 | O | GLN | A | 776 | 16.796 | 1.988 | 4.109 | 1.00 | 13.39 |
| ATOM | 1951 | N | ARG | A | 777 | 14.776 | 2.946 | 3.922 | 1.00 | 10.52 |
| ATOM | 1952 | CA | ARG | A | 777 | 15.199 | 4.197 | 4.534 | 1.00 | 11.43 |
| ATOM | 1953 | CB | ARG | A | 777 | 14.090 | 5.253 | 4.419 | 1.00 | 15.26 |
| ATOM | 1954 | CG | ARG | A | 777 | 14.487 | 6.607 | 4.948 | 1.00 | 20.06 |
| ATOM | 1955 | CD | ARG | A | 777 | 13.343 | 7.586 | 4.765 | 1.00 | 25.19 |
| ATOM | 1956 | NE | ARG | A | 777 | 12.206 | 7.273 | 5.627 | 1.00 | 34.37 |
| ATOM | 1957 | CZ | ARG | A | 777 | 12.232 | 7.359 | 6.954 | 1.00 | 35.62 |
| ATOM | 1958 | NH1 | ARG | A | 777 | 13.340 | 7.747 | 7.575 | 1.00 | 37.87 |
| ATOM | 1959 | NH2 | ARG | A | 777 | 11.149 | 7.068 | 7.661 | 1.00 | 29.66 |
| ATOM | 1960 | C | ARG | A | 777 | 15.560 | 3.979 | 5.997 | 1.00 | 10.83 |
| ATOM | 1961 | O | ARG | A | 777 | 16.601 | 4.447 | 6.472 | 1.00 | 13.15 |
| ATOM | 1962 | N | ILE | A | 778 | 14.715 | 3.238 | 6.705 | 1.00 | 12.38 |
| ATOM | 1963 | CA | ILE | A | 778 | 14.953 | 2.944 | 8.112 | 1.00 | 13.18 |
| ATOM | 1964 | CB | ILE | A | 778 | 13.753 | 2.209 | 8.744 | 1.00 | 15.50 |
| ATOM | 1965 | CG2 | ILE | A | 778 | 14.091 | 1.788 | 10.170 | 1.00 | 13.56 |
| ATOM | 1966 | CG1 | ILE | A | 778 | 12.516 | 3.108 | 8.721 | 1.00 | 22.29 |
| ATOM | 1967 | CD1 | ILE | A | 778 | 12.685 | 4.399 | 9.478 | 1.00 | 26.81 |
| ATOM | 1968 | C | ILE | A | 778 | 16.198 | 2.086 | 8.305 | 1.00 | 12.49 |
| ATOM | 1969 | O | ILE | A | 778 | 16.963 | 2.298 | 9.245 | 1.00 | 12.70 |
| ATOM | 1970 | N | ALA | A | 779 | 16.399 | 1.117 | 7.417 | 1.00 | 12.47 |
| ATOM | 1971 | CA | ALA | A | 779 | 17.563 | 0.242 | 7.510 | 1.00 | 13.19 |
| ATOM | 1972 | CB | ALA | A | 779 | 17.538 | −0.789 | 6.391 | 1.00 | 14.05 |
| ATOM | 1973 | C | ALA | A | 779 | 18.856 | 1.051 | 7.452 | 1.00 | 13.71 |
| ATOM | 1974 | O | ALA | A | 779 | 19.839 | 0.700 | 8.106 | 1.00 | 11.99 |
| ATOM | 1975 | N | GLU | A | 780 | 18.858 | 2.131 | 6.672 | 1.00 | 14.86 |
| ATOM | 1976 | CA | GLU | A | 780 | 20.048 | 2.976 | 6.560 | 1.00 | 13.62 |
| ATOM | 1977 | CB | GLU | A | 780 | 19.860 | 4.053 | 5.487 | 1.00 | 17.29 |
| ATOM | 1978 | CG | GLU | A | 780 | 19.742 | 3.519 | 4.075 | 1.00 | 30.45 |
| ATOM | 1979 | CD | GLU | A | 780 | 19.744 | 4.628 | 3.038 | 1.00 | 42.90 |
| ATOM | 1980 | OE1 | GLU | A | 780 | 20.761 | 5.347 | 2.939 | 1.00 | 42.08 |
| ATOM | 1981 | OE2 | GLU | A | 780 | 18.728 | 4.783 | 2.326 | 1.00 | 56.88 |
| ATOM | 1982 | C | GLU | A | 780 | 20.350 | 3.649 | 7.897 | 1.00 | 13.45 |
| ATOM | 1983 | O | GLU | A | 780 | 21.506 | 3.791 | 8.277 | 1.00 | 14.75 |
| ATOM | 1984 | N | LEU | A | 781 | 19.303 | 4.074 | 8.597 | 1.00 | 12.82 |
| ATOM | 1985 | CA | LEU | A | 781 | 19.464 | 4.716 | 9.898 | 1.00 | 10.66 |
| ATOM | 1986 | CB | LEU | A | 781 | 18.113 | 5.215 | 10.410 | 1.00 | 12.60 |
| ATOM | 1987 | CG | LEU | A | 781 | 17.428 | 6.296 | 9.574 | 1.00 | 15.49 |
| ATOM | 1988 | CD1 | LEU | A | 781 | 16.101 | 6.670 | 10.215 | 1.00 | 14.52 |
| ATOM | 1989 | CD2 | LEU | A | 781 | 18.339 | 7.513 | 9.475 | 1.00 | 17.00 |
| ATOM | 1990 | C | LEU | A | 781 | 20.032 | 3.704 | 10.887 | 1.00 | 12.39 |
| ATOM | 1991 | O | LEU | A | 781 | 20.935 | 4.014 | 11.673 | 1.00 | 11.98 |
| ATOM | 1992 | N | VAL | A | 782 | 19.490 | 2.490 | 10.839 | 1.00 | 9.90 |
| ATOM | 1993 | CA | VAL | A | 782 | 19.933 | 1.418 | 11.719 | 1.00 | 9.65 |
| ATOM | 1994 | CB | VAL | A | 782 | 19.116 | 0.126 | 11.463 | 1.00 | 10.42 |
| ATOM | 1995 | CG1 | VAL | A | 782 | 19.714 | −1.029 | 12.240 | 1.00 | 13.17 |
| ATOM | 1996 | CG2 | VAL | A | 782 | 17.657 | 0.339 | 11.868 | 1.00 | 16.63 |
| ATOM | 1997 | C | VAL | A | 782 | 21.418 | 1.154 | 11.477 | 1.00 | 10.70 |
| ATOM | 1998 | O | VAL | A | 782 | 22.210 | 1.068 | 12.418 | 1.00 | 13.37 |
| ATOM | 1999 | N | ALA | A | 783 | 21.793 | 1.053 | 10.206 | 1.00 | 11.25 |
| ATOM | 2000 | CA | ALA | A | 783 | 23.185 | 0.810 | 9.839 | 1.00 | 11.83 |
| ATOM | 2001 | CB | ALA | A | 783 | 23.299 | 0.632 | 8.337 | 1.00 | 15.10 |
| ATOM | 2002 | C | ALA | A | 783 | 24.074 | 1.961 | 10.298 | 1.00 | 12.03 |
| ATOM | 2003 | O | ALA | A | 783 | 25.170 | 1.746 | 10.820 | 1.00 | 12.81 |
| ATOM | 2004 | N | THR | A | 784 | 23.599 | 3.187 | 10.099 | 1.00 | 11.94 |
| ATOM | 2005 | CA | THR | A | 784 | 24.358 | 4.359 | 10.504 | 1.00 | 10.90 |
| ATOM | 2006 | CB | THR | A | 784 | 23.572 | 5.659 | 10.211 | 1.00 | 12.78 |
| ATOM | 2007 | OG1 | THR | A | 784 | 23.359 | 5.778 | 8.797 | 1.00 | 15.62 |
| ATOM | 2008 | CG2 | THR | A | 784 | 24.341 | 6.883 | 10.706 | 1.00 | 13.70 |
| ATOM | 2009 | C | THR | A | 784 | 24.676 | 4.282 | 11.995 | 1.00 | 11.74 |
| ATOM | 2010 | O | THR | A | 784 | 25.819 | 4.490 | 12.408 | 1.00 | 10.51 |
| ATOM | 2011 | N | GLU | A | 785 | 23.670 | 3.969 | 12.805 | 1.00 | 10.01 |
| ATOM | 2012 | CA | GLU | A | 785 | 23.881 | 3.880 | 14.243 | 1.00 | 10.75 |
| ATOM | 2013 | CB | GLU | A | 785 | 22.549 | 3.669 | 14.968 | 1.00 | 7.51 |
| ATOM | 2014 | CG | GLU | A | 785 | 22.675 | 3.740 | 16.483 | 1.00 | 9.65 |
| ATOM | 2015 | CD | GLU | A | 785 | 21.335 | 3.755 | 17.178 | 1.00 | 7.52 |
| ATOM | 2016 | OE1 | GLU | A | 785 | 20.415 | 4.436 | 16.674 | 1.00 | 10.93 |
| ATOM | 2017 | OE2 | GLU | A | 785 | 21.208 | 3.099 | 18.236 | 1.00 | 12.56 |
| ATOM | 2018 | C | GLU | A | 785 | 24.847 | 2.760 | 14.612 | 1.00 | 11.99 |
| ATOM | 2019 | O | GLU | A | 785 | 25.737 | 2.948 | 15.440 | 1.00 | 11.68 |
| ATOM | 2020 | N | PHE | A | 786 | 24.671 | 1.594 | 13.997 | 1.00 | 9.83 |
| ATOM | 2021 | CA | PHE | A | 786 | 25.541 | 0.464 | 14.285 | 1.00 | 10.39 |
| ATOM | 2022 | CB | PHE | A | 786 | 25.069 | −0.794 | 13.550 | 1.00 | 11.58 |
| ATOM | 2023 | CG | PHE | A | 786 | 23.876 | −1.455 | 14.176 | 1.00 | 16.59 |
| ATOM | 2024 | CD1 | PHE | A | 786 | 23.632 | −1.327 | 15.533 | 1.00 | 18.18 |
| ATOM | 2025 | CD2 | PHE | A | 786 | 23.034 | −2.254 | 13.419 | 1.00 | 19.90 |
| ATOM | 2026 | CE1 | PHE | A | 786 | 22.570 | −1.986 | 16.125 | 1.00 | 25.35 |
| ATOM | 2027 | CE2 | PHE | A | 786 | 21.972 | −2.916 | 14.004 | 1.00 | 17.12 |
| ATOM | 2028 | CZ | PHE | A | 786 | 21.740 | −2.781 | 15.360 | 1.00 | 16.84 |
| ATOM | 2029 | C | PHE | A | 786 | 26.985 | 0.760 | 13.898 | 1.00 | 9.62 |
| ATOM | 2030 | O | PHE | A | 786 | 27.907 | 0.444 | 14.652 | 1.00 | 10.46 |
| ATOM | 2031 | N | PHE | A | 787 | 27.183 | 1.348 | 12.720 | 1.00 | 8.90 |
| ATOM | 2032 | CA | PHE | A | 787 | 28.531 | 1.680 | 12.273 | 1.00 | 9.83 |
| ATOM | 2033 | CB | PHE | A | 787 | 28.515 | 2.141 | 10.809 | 1.00 | 10.96 |
| ATOM | 2034 | CG | PHE | A | 787 | 28.116 | 1.056 | 9.849 | 1.00 | 8.28 |
| ATOM | 2035 | CD1 | PHE | A | 787 | 28.335 | −0.276 | 10.172 | 1.00 | 10.72 |
| ATOM | 2036 | CD2 | PHE | A | 787 | 27.546 | 1.357 | 8.626 | 1.00 | 11.47 |
| ATOM | 2037 | CE1 | PHE | A | 787 | 27.993 | −1.287 | 9.296 | 1.00 | 12.45 |
| ATOM | 2038 | CE2 | PHE | A | 787 | 27.201 | 0.344 | 7.739 | 1.00 | 12.21 |
| ATOM | 2039 | CZ | PHE | A | 787 | 27.424 | −0.977 | 8.075 | 1.00 | 8.97 |
| ATOM | 2040 | C | PHE | A | 787 | 29.165 | 2.747 | 13.149 | 1.00 | 12.00 |
| ATOM | 2041 | O | PHE | A | 787 | 30.387 | 2.787 | 13.311 | 1.00 | 11.70 |
| ATOM | 2042 | N | ASP | A | 788 | 28.340 | 3.620 | 13.713 | 1.00 | 10.51 |
| ATOM | 2043 | CA | ASP | A | 788 | 28.867 | 4.655 | 14.583 | 1.00 | 13.07 |
| ATOM | 2044 | CB | ASP | A | 788 | 27.770 | 5.654 | 14.953 | 1.00 | 9.28 |
| ATOM | 2045 | CG | ASP | A | 788 | 28.275 | 6.751 | 15.869 | 1.00 | 17.65 |
| ATOM | 2046 | OD1 | ASP | A | 788 | 27.862 | 6.784 | 17.046 | 1.00 | 22.26 |
| ATOM | 2047 | OD2 | ASP | A | 788 | 29.097 | 7.575 | 15.414 | 1.00 | 23.70 |
| ATOM | 2048 | C | ASP | A | 788 | 29.427 | 3.975 | 15.832 | 1.00 | 11.61 |
| ATOM | 2049 | O | ASP | A | 788 | 30.491 | 4.349 | 16.324 | 1.00 | 11.23 |
| ATOM | 2050 | N | GLN | A | 789 | 28.719 | 2.965 | 16.338 | 1.00 | 10.39 |
| ATOM | 2051 | CA | GLN | A | 789 | 29.201 | 2.243 | 17.515 | 1.00 | 10.66 |
| ATOM | 2052 | CB | GLN | A | 789 | 28.185 | 1.199 | 17.979 | 1.00 | 14.04 |
| ATOM | 2053 | CG | GLN | A | 789 | 28.667 | 0.416 | 19.191 | 1.00 | 13.95 |
| ATOM | 2054 | CD | GLN | A | 789 | 27.696 | −0.652 | 19.641 | 1.00 | 13.88 |
| ATOM | 2055 | OE1 | GLN | A | 789 | 26.488 | −0.445 | 19.641 | 1.00 | 10.68 |
| ATOM | 2056 | NE2 | GLN | A | 789 | 28.226 | −1.799 | 20.051 | 1.00 | 10.88 |
| ATOM | 2057 | C | GLN | A | 789 | 30.509 | 1.541 | 17.155 | 1.00 | 12.25 |
| ATOM | 2058 | O | GLN | A | 789 | 31.452 | 1.504 | 17.952 | 1.00 | 12.92 |
| ATOM | 2059 | N | GLY | A | 790 | 30.552 | 0.979 | 15.950 | 1.00 | 12.27 |
| ATOM | 2060 | CA | GLY | A | 790 | 31.746 | 0.292 | 15.493 | 1.00 | 13.69 |
| ATOM | 2061 | C | GLY | A | 790 | 32.948 | 1.218 | 15.519 | 1.00 | 12.41 |
| ATOM | 2062 | O | GLY | A | 790 | 34.050 | 0.805 | 15.885 | 1.00 | 14.19 |
| ATOM | 2063 | N | ASP | A | 791 | 32.743 | 2.476 | 15.135 | 1.00 | 13.24 |
| ATOM | 2064 | CA | ASP | A | 791 | 33.837 | 3.448 | 15.135 | 1.00 | 15.89 |
| ATOM | 2065 | CB | ASP | A | 791 | 33.383 | 4.802 | 14.582 | 1.00 | 11.78 |
| ATOM | 2066 | CG | ASP | A | 791 | 33.022 | 4.748 | 13.108 | 1.00 | 11.72 |
| ATOM | 2067 | OD1 | ASP | A | 791 | 33.525 | 3.853 | 12.401 | 1.00 | 16.07 |
| ATOM | 2068 | OD2 | ASP | A | 791 | 32.249 | 5.619 | 12.657 | 1.00 | 16.56 |
| ATOM | 2069 | C | ASP | A | 791 | 34.350 | 3.653 | 16.554 | 1.00 | 15.06 |
| ATOM | 2070 | O | ASP | A | 791 | 35.555 | 3.751 | 16.794 | 1.00 | 15.97 |
| ATOM | 2071 | N | ARG | A | 792 | 33.423 | 3.723 | 17.496 | 1.00 | 16.00 |
| ATOM | 2072 | CA | ARG | A | 792 | 33.783 | 3.925 | 18.885 | 1.00 | 13.38 |
| ATOM | 2073 | CB | ARG | A | 792 | 32.544 | 4.308 | 19.680 | 1.00 | 15.67 |
| ATOM | 2074 | CG | ARG | A | 792 | 32.056 | 5.689 | 19.304 | 1.00 | 20.98 |
| ATOM | 2075 | CD | ARG | A | 792 | 30.791 | 6.043 | 20.014 | 1.00 | 22.55 |
| ATOM | 2076 | NE | ARG | A | 792 | 30.929 | 6.004 | 21.465 | 1.00 | 21.96 |
| ATOM | 2077 | CZ | ARG | A | 792 | 29.945 | 6.332 | 22.290 | 1.00 | 19.00 |
| ATOM | 2078 | NH1 | ARG | A | 792 | 28.782 | 6.726 | 21.791 | 1.00 | 18.95 |
| ATOM | 2079 | NH2 | ARG | A | 792 | 30.105 | 6.236 | 23.601 | 1.00 | 23.88 |
| ATOM | 2080 | C | ARG | A | 792 | 34.466 | 2.716 | 19.501 | 1.00 | 13.13 |
| ATOM | 2081 | O | ARG | A | 792 | 35.296 | 2.865 | 20.390 | 1.00 | 14.33 |
| ATOM | 2082 | N | GLU | A | 793 | 34.112 | 1.522 | 19.036 | 1.00 | 14.11 |
| ATOM | 2083 | CA | GLU | A | 793 | 34.730 | 0.305 | 19.545 | 1.00 | 12.55 |
| ATOM | 2084 | CB | GLU | A | 793 | 33.969 | −0.932 | 19.051 | 1.00 | 12.93 |
| ATOM | 2085 | CG | GLU | A | 793 | 32.531 | −0.986 | 19.543 | 1.00 | 15.09 |
| ATOM | 2086 | CD | GLU | A | 793 | 31.816 | −2.269 | 19.165 | 1.00 | 17.10 |
| ATOM | 2087 | OE1 | GLU | A | 793 | 32.105 | −2.807 | 18.078 | 1.00 | 14.75 |
| ATOM | 2088 | OE2 | GLU | A | 793 | 30.951 | −2.728 | 19.949 | 1.00 | 13.37 |
| ATOM | 2089 | C | GLU | A | 793 | 36.184 | 0.251 | 19.080 | 1.00 | 13.70 |
| ATOM | 2090 | O | GLU | A | 793 | 37.073 | −0.143 | 19.837 | 1.00 | 14.48 |
| ATOM | 2091 | N | ARG | A | 794 | 36.429 | 0.655 | 17.838 | 1.00 | 12.88 |
| ATOM | 2092 | CA | ARG | A | 794 | 37.792 | 0.647 | 17.318 | 1.00 | 16.50 |
| ATOM | 2093 | CB | ARG | A | 794 | 37.828 | 1.083 | 15.847 | 1.00 | 16.91 |
| ATOM | 2094 | CG | ARG | A | 794 | 37.330 | 0.047 | 14.838 | 1.00 | 16.40 |
| ATOM | 2095 | CD | ARG | A | 794 | 37.399 | 0.609 | 13.422 | 1.00 | 20.95 |
| ATOM | 2096 | NE | ARG | A | 794 | 36.790 | −0.268 | 12.422 | 1.00 | 21.25 |
| ATOM | 2097 | CZ | ARG | A | 794 | 37.323 | −1.407 | 11.996 | 1.00 | 16.41 |
| ATOM | 2098 | NH1 | ARG | A | 794 | 38.486 | −1.822 | 12.480 | 1.00 | 26.91 |
| ATOM | 2099 | NH2 | ARG | A | 794 | 36.691 | −2.134 | 11.082 | 1.00 | 18.30 |
| ATOM | 2100 | C | ARG | A | 794 | 38.648 | 1.603 | 18.141 | 1.00 | 17.48 |
| ATOM | 2101 | O | ARG | A | 794 | 39.781 | 1.287 | 18.503 | 1.00 | 18.61 |
| ATOM | 2102 | N | LYS | A | 795 | 38.087 | 2.769 | 18.443 | 1.00 | 19.00 |
| ATOM | 2103 | CA | LYS | A | 795 | 38.793 | 3.795 | 19.199 | 1.00 | 19.03 |
| ATOM | 2104 | CB | LYS | A | 795 | 38.146 | 5.161 | 18.947 | 1.00 | 22.77 |
| ATOM | 2105 | CG | LYS | A | 795 | 38.839 | 6.312 | 19.661 | 1.00 | 30.54 |
| ATOM | 2106 | CD | LYS | A | 795 | 38.128 | 7.633 | 19.410 | 1.00 | 38.45 |
| ATOM | 2107 | CE | LYS | A | 795 | 38.902 | 8.796 | 20.009 | 1.00 | 34.57 |
| ATOM | 2108 | NZ | LYS | A | 795 | 40.267 | 8.886 | 19.424 | 1.00 | 42.21 |
| ATOM | 2109 | C | LYS | A | 795 | 38.866 | 3.554 | 20.704 | 1.00 | 19.86 |
| ATOM | 2110 | O | LYS | A | 795 | 39.923 | 3.716 | 21.310 | 1.00 | 21.68 |
| ATOM | 2111 | N | GLU | A | 796 | 37.747 | 3.166 | 21.304 | 1.00 | 17.50 |
| ATOM | 2112 | CA | GLU | A | 796 | 37.696 | 2.953 | 22.745 | 1.00 | 18.20 |
| ATOM | 2113 | CB | GLU | A | 796 | 36.286 | 3.272 | 23.255 | 1.00 | 16.63 |
| ATOM | 2114 | CG | GLU | A | 796 | 35.872 | 4.722 | 23.048 | 1.00 | 19.55 |
| ATOM | 2115 | CD | GLU | A | 796 | 34.425 | 4.982 | 23.414 | 1.00 | 17.22 |
| ATOM | 2116 | OE1 | GLU | A | 796 | 33.986 | 4.520 | 24.489 | 1.00 | 17.11 |
| ATOM | 2117 | OE2 | GLU | A | 796 | 33.729 | 5.660 | 22.629 | 1.00 | 17.31 |
| ATOM | 2118 | C | GLU | A | 796 | 38.116 | 1.568 | 23.238 | 1.00 | 18.82 |
| ATOM | 2119 | O | GLU | A | 796 | 38.604 | 1.432 | 24.359 | 1.00 | 19.88 |
| ATOM | 2120 | N | LEU | A | 797 | 37.936 | 0.544 | 22.408 | 1.00 | 17.76 |
| ATOM | 2121 | CA | LEU | A | 797 | 38.279 | −0.817 | 22.809 | 1.00 | 17.55 |
| ATOM | 2122 | CB | LEU | A | 797 | 37.032 | −1.708 | 22.732 | 1.00 | 14.21 |
| ATOM | 2123 | CG | LEU | A | 797 | 35.816 | −1.230 | 23.535 | 1.00 | 21.04 |
| ATOM | 2124 | CD1 | LEU | A | 797 | 34.655 | −2.194 | 23.333 | 1.00 | 19.81 |
| ATOM | 2125 | CD2 | LEU | A | 797 | 36.177 | −1.134 | 25.008 | 1.00 | 22.47 |
| ATOM | 2126 | C | LEU | A | 797 | 39.400 | −1.441 | 21.980 | 1.00 | 18.06 |
| ATOM | 2127 | O | LEU | A | 797 | 39.899 | −2.518 | 22.312 | 1.00 | 18.10 |
| ATOM | 2128 | N | ASN | A | 798 | 39.795 | −0.760 | 20.910 | 1.00 | 17.87 |
| ATOM | 2129 | CA | ASN | A | 798 | 40.846 | −1.244 | 20.016 | 1.00 | 17.14 |
| ATOM | 2130 | CB | ASN | A | 798 | 42.202 | −1.278 | 20.732 | 1.00 | 18.53 |
| ATOM | 2131 | CG | ASN | A | 798 | 43.357 | −1.501 | 19.770 | 1.00 | 19.84 |
| ATOM | 2132 | OD1 | ASN | A | 798 | 43.497 | −0.774 | 18.788 | 1.00 | 22.65 |
| ATOM | 2133 | ND2 | ASN | A | 798 | 44.186 | −2.507 | 20.043 | 1.00 | 17.46 |
| ATOM | 2134 | C | ASN | A | 798 | 40.522 | −2.632 | 19.472 | 1.00 | 19.40 |
| ATOM | 2135 | O | ASN | A | 798 | 41.400 | −3.485 | 19.352 | 1.00 | 18.37 |
| ATOM | 2136 | N | ILE | A | 799 | 39.254 | −2.852 | 19.141 | 1.00 | 18.23 |
| ATOM | 2137 | CA | ILE | A | 799 | 38.816 | −4.134 | 18.603 | 1.00 | 20.72 |
| ATOM | 2138 | CB | ILE | A | 799 | 37.779 | −4.811 | 19.529 | 1.00 | 23.69 |
| ATOM | 2139 | CG2 | ILE | A | 799 | 38.348 | −4.970 | 20.930 | 1.00 | 25.12 |
| ATOM | 2140 | CG1 | ILE | A | 799 | 36.500 | −3.968 | 19.570 | 1.00 | 27.29 |
| ATOM | 2141 | CD1 | ILE | A | 799 | 35.381 | −4.581 | 20.373 | 1.00 | 23.09 |
| ATOM | 2142 | C | ILE | A | 799 | 38.158 | −3.926 | 17.243 | 1.00 | 20.66 |
| ATOM | 2143 | O | ILE | A | 799 | 37.636 | −2.846 | 16.956 | 1.00 | 20.48 |
| ATOM | 2144 | N | GLU | A | 800 | 38.199 | −4.951 | 16.399 | 1.00 | 20.84 |
| ATOM | 2145 | CA | GLU | A | 800 | 37.558 | −4.855 | 15.096 | 1.00 | 19.43 |
| ATOM | 2146 | CB | GLU | A | 800 | 38.162 | −5.856 | 14.109 | 1.00 | 25.89 |
| ATOM | 2147 | CG | GLU | A | 800 | 37.507 | −5.813 | 12.735 | 1.00 | 37.89 |
| ATOM | 2148 | CD | GLU | A | 800 | 38.245 | −6.637 | 11.699 | 1.00 | 48.08 |
| ATOM | 2149 | OE1 | GLU | A | 800 | 38.421 | −7.856 | 11.915 | 1.00 | 47.23 |
| ATOM | 2150 | OE2 | GLU | A | 800 | 38.647 | −6.063 | 10.663 | 1.00 | 52.27 |
| ATOM | 2151 | C | GLU | A | 800 | 36.095 | −5.188 | 15.345 | 1.00 | 16.86 |
| ATOM | 2152 | O | GLU | A | 800 | 35.774 | −6.249 | 15.879 | 1.00 | 20.00 |
| ATOM | 2153 | N | PRO | A | 801 | 35.186 | −4.277 | 14.979 | 1.00 | 16.72 |
| ATOM | 2154 | CD | PRO | A | 801 | 35.413 | −2.971 | 14.340 | 1.00 | 13.94 |
| ATOM | 2155 | CA | PRO | A | 801 | 33.755 | −4.518 | 15.188 | 1.00 | 15.17 |
| ATOM | 2156 | CB | PRO | A | 801 | 33.101 | −3.242 | 14.651 | 1.00 | 11.35 |
| ATOM | 2157 | CG | PRO | A | 801 | 34.189 | −2.204 | 14.760 | 1.00 | 18.39 |
| ATOM | 2158 | C | PRO | A | 801 | 33.249 | −5.750 | 14.446 | 1.00 | 14.88 |
| ATOM | 2159 | O | PRO | A | 801 | 33.836 | −6.168 | 13.437 | 1.00 | 14.57 |
| ATOM | 2160 | N | THR | A | 802 | 32.166 | −6.333 | 14.952 | 1.00 | 13.89 |
| ATOM | 2161 | CA | THR | A | 802 | 31.554 | −7.473 | 14.282 | 1.00 | 13.70 |
| ATOM | 2162 | CB | THR | A | 802 | 30.367 | −8.057 | 15.090 | 1.00 | 18.66 |
| ATOM | 2163 | OG1 | THR | A | 802 | 29.339 | −7.067 | 15.227 | 1.00 | 22.82 |
| ATOM | 2164 | CG2 | THR | A | 802 | 30.824 | −8.491 | 16.475 | 1.00 | 28.52 |
| ATOM | 2165 | C | THR | A | 802 | 31.028 | −6.886 | 12.974 | 1.00 | 13.06 |
| ATOM | 2166 | O | THR | A | 802 | 30.896 | −5.666 | 12.853 | 1.00 | 13.12 |
| ATOM | 2167 | N | ASP | A | 803 | 30.730 | −7.737 | 11.997 | 1.00 | 13.68 |
| ATOM | 2168 | CA | ASP | A | 803 | 30.239 | −7.260 | 10.706 | 1.00 | 13.10 |
| ATOM | 2169 | CB | ASP | A | 803 | 29.784 | −8.436 | 9.840 | 1.00 | 15.94 |
| ATOM | 2170 | CG | ASP | A | 803 | 30.942 | −9.281 | 9.340 | 1.00 | 19.70 |
| ATOM | 2171 | OD1 | ASP | A | 803 | 32.108 | −8.965 | 9.661 | 1.00 | 16.86 |
| ATOM | 2172 | OD2 | ASP | A | 803 | 30.681 | −10.265 | 8.619 | 1.00 | 17.57 |
| ATOM | 2173 | C | ASP | A | 803 | 29.092 | −6.262 | 10.829 | 1.00 | 14.26 |
| ATOM | 2174 | O | ASP | A | 803 | 29.071 | −5.239 | 10.141 | 1.00 | 15.59 |
| ATOM | 2175 | N | LEU | A | 804 | 28.142 | −6.561 | 11.706 | 1.00 | 12.76 |
| ATOM | 2176 | CA | LEU | A | 804 | 26.982 | −5.700 | 11.907 | 1.00 | 13.21 |
| ATOM | 2177 | CB | LEU | A | 804 | 26.116 | −6.251 | 13.042 | 1.00 | 14.65 |
| ATOM | 2178 | CG | LEU | A | 804 | 24.861 | −5.430 | 13.347 | 1.00 | 15.24 |
| ATOM | 2179 | CD1 | LEU | A | 804 | 24.020 | −5.317 | 12.092 | 1.00 | 16.29 |
| ATOM | 2180 | CD2 | LEU | A | 804 | 24.068 | −6.089 | 14.461 | 1.00 | 23.77 |
| ATOM | 2181 | C | LEU | A | 804 | 27.356 | −4.252 | 12.214 | 1.00 | 13.48 |
| ATOM | 2182 | O | LEU | A | 804 | 26.646 | −3.317 | 11.825 | 1.00 | 13.22 |
| ATOM | 2183 | N | MET | A | 805 | 28.476 | −4.067 | 12.904 | 1.00 | 12.28 |
| ATOM | 2184 | CA | MET | A | 805 | 28.909 | −2.725 | 13.276 | 1.00 | 11.75 |
| ATOM | 2185 | CB | MET | A | 805 | 29.141 | −2.671 | 14.790 | 1.00 | 13.28 |
| ATOM | 2186 | CG | MET | A | 805 | 27.859 | −2.865 | 15.589 | 1.00 | 33.42 |
| ATOM | 2187 | SD | MET | A | 805 | 28.125 | −3.166 | 17.338 | 1.00 | 47.91 |
| ATOM | 2188 | CE | MET | A | 805 | 26.447 | −3.244 | 17.925 | 1.00 | 28.62 |
| ATOM | 2189 | C | MET | A | 805 | 30.160 | −2.268 | 12.542 | 1.00 | 11.31 |
| ATOM | 2190 | O | MET | A | 805 | 30.761 | −1.259 | 12.908 | 1.00 | 12.42 |
| ATOM | 2191 | N | ASN | A | 806 | 30.530 | −2.992 | 11.490 | 1.00 | 11.94 |
| ATOM | 2192 | CA | ASN | A | 806 | 31.726 | −2.673 | 10.712 | 1.00 | 13.21 |
| ATOM | 2193 | CB | ASN | A | 806 | 32.537 | −3.954 | 10.471 | 1.00 | 13.42 |
| ATOM | 2194 | CG | ASN | A | 806 | 33.907 | −3.676 | 9.876 | 1.00 | 16.11 |
| ATOM | 2195 | OD1 | ASN | A | 806 | 34.209 | −2.549 | 9.484 | 1.00 | 17.92 |
| ATOM | 2196 | ND2 | ASN | A | 806 | 34.740 | −4.706 | 9.802 | 1.00 | 15.54 |
| ATOM | 2197 | C | ASN | A | 806 | 31.371 | −2.023 | 9.374 | 1.00 | 14.20 |
| ATOM | 2198 | O | ASN | A | 806 | 30.988 | −2.715 | 8.430 | 1.00 | 14.08 |
| ATOM | 2199 | N | ARG | A | 807 | 31.508 | −0.699 | 9.296 | 1.00 | 12.56 |
| ATOM | 2200 | CA | ARG | A | 807 | 31.189 | 0.037 | 8.075 | 1.00 | 14.91 |
| ATOM | 2201 | CB | ARG | A | 807 | 31.410 | 1.543 | 8.275 | 1.00 | 16.99 |
| ATOM | 2202 | CG | ARG | A | 807 | 30.891 | 2.385 | 7.116 | 1.00 | 25.83 |
| ATOM | 2203 | CD | ARG | A | 807 | 31.162 | 3.881 | 7.280 | 1.00 | 30.30 |
| ATOM | 2204 | NE | ARG | A | 807 | 30.520 | 4.470 | 8.457 | 1.00 | 20.02 |
| ATOM | 2205 | CZ | ARG | A | 807 | 31.078 | 4.525 | 9.662 | 1.00 | 16.27 |
| ATOM | 2206 | NH1 | ARG | A | 807 | 32.290 | 4.025 | 9.854 | 1.00 | 20.90 |
| ATOM | 2207 | NH2 | ARG | A | 807 | 30.431 | 5.095 | 10.670 | 1.00 | 15.75 |
| ATOM | 2208 | C | ARG | A | 807 | 32.021 | −0.442 | 6.889 | 1.00 | 13.89 |
| ATOM | 2209 | O | ARG | A | 807 | 31.573 | −0.392 | 5.736 | 1.00 | 13.84 |
| ATOM | 2210 | N | GLU | A | 808 | 33.228 | −0.910 | 7.177 | 1.00 | 11.68 |
| ATOM | 2211 | CA | GLU | A | 808 | 34.122 | −1.397 | 6.137 | 1.00 | 17.06 |
| ATOM | 2212 | CB | GLU | A | 808 | 35.502 | −1.675 | 6.731 | 1.00 | 16.87 |
| ATOM | 2213 | CG | GLU | A | 808 | 36.256 | −0.394 | 7.085 | 1.00 | 22.64 |
| ATOM | 2214 | CD | GLU | A | 808 | 37.556 | −0.645 | 7.819 | 1.00 | 24.85 |
| ATOM | 2215 | OE1 | GLU | A | 808 | 38.293 | −1.576 | 7.430 | 1.00 | 26.32 |
| ATOM | 2216 | OE2 | GLU | A | 808 | 37.846 | 0.100 | 8.780 | 1.00 | 36.15 |
| ATOM | 2217 | C | GLU | A | 808 | 33.565 | −2.641 | 5.461 | 1.00 | 15.58 |
| ATOM | 2218 | O | GLU | A | 808 | 33.929 | −2.956 | 4.329 | 1.00 | 15.69 |
| ATOM | 2219 | N | LYS | A | 809 | 32.677 | −3.343 | 6.156 | 1.00 | 14.84 |
| ATOM | 2220 | CA | LYS | A | 809 | 32.062 | −4.544 | 5.600 | 1.00 | 16.67 |
| ATOM | 2221 | CB | LYS | A | 809 | 32.381 | −5.765 | 6.478 | 1.00 | 12.30 |
| ATOM | 2222 | CG | LYS | A | 809 | 33.845 | −6.184 | 6.405 | 1.00 | 13.87 |
| ATOM | 2223 | CD | LYS | A | 809 | 34.151 | −7.408 | 7.259 | 1.00 | 14.82 |
| ATOM | 2224 | CE | LYS | A | 809 | 33.430 | −8.641 | 6.745 | 1.00 | 25.64 |
| ATOM | 2225 | NZ | LYS | A | 809 | 33.789 | −9.854 | 7.534 | 1.00 | 21.95 |
| ATOM | 2226 | C | LYS | A | 809 | 30.555 | −4.363 | 5.445 | 1.00 | 15.95 |
| ATOM | 2227 | O | LYS | A | 809 | 29.770 | −5.281 | 5.693 | 1.00 | 18.05 |
| ATOM | 2228 | N | LYS | A | 810 | 30.156 | −3.167 | 5.022 | 1.00 | 15.13 |
| ATOM | 2229 | CA | LYS | A | 810 | 28.742 | −2.867 | 4.828 | 1.00 | 16.24 |
| ATOM | 2230 | CB | LYS | A | 810 | 28.549 | −1.383 | 4.494 | 1.00 | 14.35 |
| ATOM | 2231 | CG | LYS | A | 810 | 29.347 | −0.869 | 3.312 | 1.00 | 24.48 |
| ATOM | 2232 | CD | LYS | A | 810 | 29.222 | 0.647 | 3.226 | 1.00 | 29.64 |
| ATOM | 2233 | CE | LYS | A | 810 | 29.994 | 1.224 | 2.049 | 1.00 | 35.68 |
| ATOM | 2234 | NZ | LYS | A | 810 | 29.432 | 0.786 | 0.746 | 1.00 | 42.39 |
| ATOM | 2235 | C | LYS | A | 810 | 28.139 | −3.744 | 3.734 | 1.00 | 14.42 |
| ATOM | 2236 | O | LYS | A | 810 | 26.921 | −3.843 | 3.607 | 1.00 | 14.44 |
| ATOM | 2237 | N | ASN | A | 811 | 28.995 | −4.389 | 2.950 | 1.00 | 13.62 |
| ATOM | 2238 | CA | ASN | A | 811 | 28.511 | −5.270 | 1.893 | 1.00 | 13.60 |
| ATOM | 2239 | CB | ASN | A | 811 | 29.680 | −5.746 | 1.016 | 1.00 | 18.14 |
| ATOM | 2240 | CG | ASN | A | 811 | 30.815 | −6.340 | 1.826 | 1.00 | 21.65 |
| ATOM | 2241 | OD1 | ASN | A | 811 | 30.936 | −7.559 | 1.958 | 1.00 | 17.54 |
| ATOM | 2242 | ND2 | ASN | A | 811 | 31.651 | −5.472 | 2.388 | 1.00 | 13.93 |
| ATOM | 2243 | C | ASN | A | 811 | 27.777 | −6.474 | 2.487 | 1.00 | 14.60 |
| ATOM | 2244 | O | ASN | A | 811 | 27.084 | −7.197 | 1.777 | 1.00 | 13.25 |
| ATOM | 2245 | N | LYS | A | 812 | 27.922 | −6.677 | 3.796 | 1.00 | 14.05 |
| ATOM | 2246 | CA | LYS | A | 812 | 27.279 | −7.797 | 4.478 | 1.00 | 15.17 |
| ATOM | 2247 | CB | LYS | A | 812 | 28.057 | −8.150 | 5.753 | 1.00 | 15.23 |
| ATOM | 2248 | CG | LYS | A | 812 | 29.401 | −8.803 | 5.500 | 1.00 | 20.27 |
| ATOM | 2249 | CD | LYS | A | 812 | 29.198 | −10.137 | 4.817 | 1.00 | 28.44 |
| ATOM | 2250 | CE | LYS | A | 812 | 30.497 | −10.886 | 4.658 | 1.00 | 35.44 |
| ATOM | 2251 | NZ | LYS | A | 812 | 30.257 | −12.223 | 4.048 | 1.00 | 38.51 |
| ATOM | 2252 | C | LYS | A | 812 | 25.818 | −7.542 | 4.859 | 1.00 | 15.78 |
| ATOM | 2253 | O | LYS | A | 812 | 25.109 | −8.475 | 5.258 | 1.00 | 13.98 |
| ATOM | 2254 | N | ILE | A | 813 | 25.366 | −6.297 | 4.724 | 1.00 | 14.64 |
| ATOM | 2255 | CA | ILE | A | 813 | 24.012 | −5.964 | 5.144 | 1.00 | 14.83 |
| ATOM | 2256 | CB | ILE | A | 813 | 23.770 | −4.460 | 5.007 | 1.00 | 15.21 |
| ATOM | 2257 | CG2 | ILE | A | 813 | 22.264 | −4.158 | 5.091 | 1.00 | 15.81 |
| ATOM | 2258 | CG1 | ILE | A | 813 | 24.616 | −3.740 | 6.073 | 1.00 | 13.99 |
| ATOM | 2259 | CD1 | ILE | A | 813 | 24.445 | −2.265 | 6.070 | 1.00 | 27.43 |
| ATOM | 2260 | C | ILE | A | 813 | 22.862 | −6.792 | 4.546 | 1.00 | 14.67 |
| ATOM | 2261 | O | ILE | A | 813 | 22.042 | −7.309 | 5.270 | 1.00 | 13.44 |
| ATOM | 2262 | N | PRO | A | 814 | 22.807 | −6.936 | 3.224 | 1.00 | 12.07 |
| ATOM | 2263 | CD | PRO | A | 814 | 23.603 | −6.274 | 2.191 | 1.00 | 14.86 |
| ATOM | 2264 | CA | PRO | A | 814 | 21.709 | −7.724 | 2.623 | 1.00 | 14.39 |
| ATOM | 2265 | CB | PRO | A | 814 | 22.060 | −7.714 | 1.152 | 1.00 | 13.45 |
| ATOM | 2266 | CG | PRO | A | 814 | 22.693 | −6.351 | 1.001 | 1.00 | 14.67 |
| ATOM | 2267 | C | PRO | A | 814 | 21.605 | −9.144 | 3.185 | 1.00 | 11.82 |
| ATOM | 2268 | O | PRO | A | 814 | 20.525 | −9.603 | 3.570 | 1.00 | 12.87 |
| ATOM | 2269 | N | SER | A | 815 | 22.730 | −9.846 | 3.215 | 1.00 | 11.71 |
| ATOM | 2270 | CA | SER | A | 815 | 22.730 | −11.205 | 3.729 | 1.00 | 13.13 |
| ATOM | 2271 | CB | SER | A | 815 | 24.102 | −11.863 | 3.526 | 1.00 | 14.66 |
| ATOM | 2272 | OG | SER | A | 815 | 25.124 | −11.185 | 4.233 | 1.00 | 27.24 |
| ATOM | 2273 | C | SER | A | 815 | 22.366 | −11.220 | 5.212 | 1.00 | 14.09 |
| ATOM | 2274 | O | SER | A | 815 | 21.677 | −12.120 | 5.682 | 1.00 | 14.61 |
| ATOM | 2275 | N | MET | A | 816 | 22.809 | −10.211 | 5.937 | 1.00 | 14.82 |
| ATOM | 2276 | CA | MET | A | 816 | 22.498 | −10.197 | 7.353 | 1.00 | 14.08 |
| ATOM | 2277 | CB | MET | A | 816 | 23.449 | −9.254 | 8.079 | 1.00 | 16.95 |
| ATOM | 2278 | CG | MET | A | 816 | 24.829 | −9.872 | 8.232 | 1.00 | 19.08 |
| ATOM | 2279 | SD | MET | A | 816 | 26.019 | −8.905 | 9.168 | 1.00 | 23.16 |
| ATOM | 2280 | CE | MET | A | 816 | 25.502 | −9.310 | 10.812 | 1.00 | 29.41 |
| ATOM | 2281 | C | MET | A | 816 | 21.057 | −9.838 | 7.628 | 1.00 | 12.78 |
| ATOM | 2282 | O | MET | A | 816 | 20.477 | −10.281 | 8.613 | 1.00 | 13.06 |
| ATOM | 2283 | N | GLN | A | 817 | 20.464 | −9.010 | 6.765 | 1.00 | 11.62 |
| ATOM | 2284 | CA | GLN | A | 817 | 19.061 | −8.637 | 6.915 | 1.00 | 11.33 |
| ATOM | 2285 | CB | GLN | A | 817 | 18.666 | −7.547 | 5.899 | 1.00 | 12.94 |
| ATOM | 2286 | CG | GLN | A | 817 | 19.186 | −6.179 | 6.283 | 1.00 | 11.95 |
| ATOM | 2287 | CD | GLN | A | 817 | 18.469 | −5.624 | 7.500 | 1.00 | 17.94 |
| ATOM | 2288 | OE1 | GLN | A | 817 | 18.544 | −6.183 | 8.595 | 1.00 | 34.56 |
| ATOM | 2289 | NE2 | GLN | A | 817 | 17.759 | −4.523 | 7.309 | 1.00 | 34.13 |
| ATOM | 2290 | C | GLN | A | 817 | 18.202 | −9.882 | 6.714 | 1.00 | 13.31 |
| ATOM | 2291 | O | GLN | A | 817 | 17.254 | −10.116 | 7.472 | 1.00 | 10.14 |
| ATOM | 2292 | N | VAL | A | 818 | 18.537 | −10.698 | 5.715 | 1.00 | 12.18 |
| ATOM | 2293 | CA | VAL | A | 818 | 17.774 | −11.918 | 5.479 | 1.00 | 11.53 |
| ATOM | 2294 | CB | VAL | A | 818 | 18.252 | −12.659 | 4.210 | 1.00 | 13.56 |
| ATOM | 2295 | CG1 | VAL | A | 818 | 17.500 | −13.972 | 4.063 | 1.00 | 10.58 |
| ATOM | 2296 | CG2 | VAL | A | 818 | 18.032 | −11.781 | 2.982 | 1.00 | 17.92 |
| ATOM | 2297 | C | VAL | A | 818 | 17.903 | −12.858 | 6.678 | 1.00 | 11.51 |
| ATOM | 2298 | O | VAL | A | 818 | 16.930 | −13.494 | 7.090 | 1.00 | 12.55 |
| ATOM | 2299 | N | GLY | A | 819 | 19.110 | −12.934 | 7.231 | 1.00 | 11.44 |
| ATOM | 2300 | CA | GLY | A | 819 | 19.356 | −13.791 | 8.376 | 1.00 | 12.74 |
| ATOM | 2301 | C | GLY | A | 819 | 18.556 | −13.335 | 9.579 | 1.00 | 13.96 |
| ATOM | 2302 | O | GLY | A | 819 | 18.068 | −14.149 | 10.360 | 1.00 | 13.49 |
| ATOM | 2303 | N | PHE | A | 820 | 18.430 | −12.022 | 9.725 | 1.00 | 14.20 |
| ATOM | 2304 | CA | PHE | A | 820 | 17.682 | −11.426 | 10.825 | 1.00 | 11.70 |
| ATOM | 2305 | CB | PHE | A | 820 | 17.927 | −9.913 | 10.833 | 1.00 | 11.32 |
| ATOM | 2306 | CG | PHE | A | 820 | 17.122 | −9.167 | 11.858 | 1.00 | 9.99 |
| ATOM | 2307 | CD1 | PHE | A | 820 | 17.268 | −9.436 | 13.209 | 1.00 | 23.52 |
| ATOM | 2308 | CD2 | PHE | A | 820 | 16.225 | −8.184 | 11.466 | 1.00 | 17.26 |
| ATOM | 2309 | CE1 | PHE | A | 820 | 16.529 | −8.740 | 14.151 | 1.00 | 19.87 |
| ATOM | 2310 | CE2 | PHE | A | 820 | 15.485 | −7.488 | 12.403 | 1.00 | 16.59 |
| ATOM | 2311 | CZ | PHE | A | 820 | 15.640 | −7.767 | 13.746 | 1.00 | 17.66 |
| ATOM | 2312 | C | PHE | A | 820 | 16.191 | −11.731 | 10.651 | 1.00 | 12.70 |
| ATOM | 2313 | O | PHE | A | 820 | 15.498 | −12.103 | 11.604 | 1.00 | 10.72 |
| ATOM | 2314 | N | ILE | A | 821 | 15.697 | −11.571 | 9.429 | 1.00 | 10.04 |
| ATOM | 2315 | CA | ILE | A | 821 | 14.298 | −11.850 | 9.150 | 1.00 | 9.29 |
| ATOM | 2316 | CB | ILE | A | 821 | 13.957 | −11.540 | 7.679 | 1.00 | 10.14 |
| ATOM | 2317 | CG2 | ILE | A | 821 | 12.617 | −12.166 | 7.307 | 1.00 | 12.87 |
| ATOM | 2318 | CG1 | ILE | A | 821 | 13.931 | −10.022 | 7.479 | 1.00 | 12.03 |
| ATOM | 2319 | CD1 | ILE | A | 821 | 13.795 | −9.580 | 6.037 | 1.00 | 14.77 |
| ATOM | 2320 | C | ILE | A | 821 | 13.991 | −13.311 | 9.462 | 1.00 | 11.67 |
| ATOM | 2321 | O | ILE | A | 821 | 13.012 | −13.617 | 10.140 | 1.00 | 11.27 |
| ATOM | 2322 | N | ASP | A | 822 | 14.841 | −14.215 | 8.985 | 1.00 | 11.97 |
| ATOM | 2323 | CA | ASP | A | 822 | 14.634 | −15.637 | 9.237 | 1.00 | 14.30 |
| ATOM | 2324 | CB | ASP | A | 822 | 15.630 | −16.479 | 8.428 | 1.00 | 17.59 |
| ATOM | 2325 | CG | ASP | A | 822 | 15.317 | −16.499 | 6.942 | 1.00 | 17.92 |
| ATOM | 2326 | OD1 | ASP | A | 822 | 14.177 | −16.153 | 6.564 | 1.00 | 15.98 |
| ATOM | 2327 | OD2 | ASP | A | 822 | 16.212 | −16.880 | 6.152 | 1.00 | 13.97 |
| ATOM | 2328 | C | ASP | A | 822 | 14.759 | −16.021 | 10.714 | 1.00 | 15.42 |
| ATOM | 2329 | O | ASP | A | 822 | 13.876 | −16.671 | 11.275 | 1.00 | 16.53 |
| ATOM | 2330 | N | ALA | A | 823 | 15.849 | −15.608 | 11.345 | 1.00 | 13.55 |
| ATOM | 2331 | CA | ALA | A | 823 | 16.101 | −15.971 | 12.737 | 1.00 | 15.03 |
| ATOM | 2332 | CB | ALA | A | 823 | 17.580 | −15.756 | 13.058 | 1.00 | 13.84 |
| ATOM | 2333 | C | ALA | A | 823 | 15.254 | −15.299 | 13.810 | 1.00 | 15.13 |
| ATOM | 2334 | O | ALA | A | 823 | 14.907 | −15.928 | 14.810 | 1.00 | 17.91 |
| ATOM | 2335 | N | ILE | A | 824 | 14.914 | −14.032 | 13.606 | 1.00 | 13.91 |
| ATOM | 2336 | CA | ILE | A | 824 | 14.169 | −13.281 | 14.611 | 1.00 | 14.87 |
| ATOM | 2337 | CB | ILE | A | 824 | 14.929 | −11.971 | 14.966 | 1.00 | 16.51 |
| ATOM | 2338 | CG2 | ILE | A | 824 | 14.085 | −11.106 | 15.891 | 1.00 | 11.92 |
| ATOM | 2339 | CG1 | ILE | A | 824 | 16.291 | −12.293 | 15.589 | 1.00 | 19.03 |
| ATOM | 2340 | CD1 | ILE | A | 824 | 16.211 | −12.966 | 16.938 | 1.00 | 17.79 |
| ATOM | 2341 | C | ILE | A | 824 | 12.736 | −12.872 | 14.271 | 1.00 | 15.38 |
| ATOM | 2342 | O | ILE | A | 824 | 11.817 | −13.051 | 15.077 | 1.00 | 15.58 |
| ATOM | 2343 | N | CYS | A | 825 | 12.548 | −12.332 | 13.074 | 1.00 | 11.66 |
| ATOM | 2344 | CA | CYS | A | 825 | 11.248 | −11.798 | 12.682 | 1.00 | 10.83 |
| ATOM | 2345 | CB | CYS | A | 825 | 11.470 | −10.717 | 11.629 | 1.00 | 14.06 |
| ATOM | 2346 | SG | CYS | A | 825 | 12.684 | −9.498 | 12.156 | 1.00 | 12.72 |
| ATOM | 2347 | C | CYS | A | 825 | 10.094 | −12.685 | 12.231 | 1.00 | 12.83 |
| ATOM | 2348 | O | CYS | A | 825 | 9.003 | −12.606 | 12.792 | 1.00 | 11.78 |
| ATOM | 2349 | N | LEU | A | 826 | 10.312 | −13.508 | 11.213 | 1.00 | 10.64 |
| ATOM | 2350 | CA | LEU | A | 826 | 9.230 | −14.343 | 10.699 | 1.00 | 12.29 |
| ATOM | 2351 | CB | LEU | A | 826 | 9.759 | −15.291 | 9.621 | 1.00 | 17.45 |
| ATOM | 2352 | CG | LEU | A | 826 | 10.132 | −14.585 | 8.314 | 1.00 | 14.44 |
| ATOM | 2353 | CD1 | LEU | A | 826 | 10.756 | −15.585 | 7.355 | 1.00 | 19.69 |
| ATOM | 2354 | CD2 | LEU | A | 826 | 8.884 | −13.965 | 7.693 | 1.00 | 17.13 |
| ATOM | 2355 | C | LEU | A | 826 | 8.428 | −15.131 | 11.735 | 1.00 | 11.75 |
| ATOM | 2356 | O | LEU | A | 826 | 7.194 | −15.094 | 11.728 | 1.00 | 12.87 |
| ATOM | 2357 | N | GLN | A | 827 | 9.115 | −15.842 | 12.620 | 1.00 | 9.56 |
| ATOM | 2358 | CA | GLN | A | 827 | 8.426 | −16.637 | 13.632 | 1.00 | 10.96 |
| ATOM | 2359 | CB | GLN | A | 827 | 9.438 | −17.368 | 14.507 | 1.00 | 9.95 |
| ATOM | 2360 | CG | GLN | A | 827 | 10.207 | −18.465 | 13.805 | 1.00 | 14.60 |
| ATOM | 2361 | CD | GLN | A | 827 | 11.365 | −18.949 | 14.646 | 1.00 | 27.21 |
| ATOM | 2362 | OE1 | GLN | A | 827 | 12.420 | −18.312 | 14.695 | 1.00 | 20.68 |
| ATOM | 2363 | NE2 | GLN | A | 827 | 11.169 | −20.068 | 15.335 | 1.00 | 24.19 |
| ATOM | 2364 | C | GLN | A | 827 | 7.516 | −15.798 | 14.524 | 1.00 | 13.14 |
| ATOM | 2365 | O | GLN | A | 827 | 6.428 | −16.235 | 14.906 | 1.00 | 13.21 |
| ATOM | 2366 | N | LEU | A | 828 | 7.966 | −14.596 | 14.862 | 1.00 | 10.02 |
| ATOM | 2367 | CA | LEU | A | 828 | 7.182 | −13.713 | 15.720 | 1.00 | 10.78 |
| ATOM | 2368 | CB | LEU | A | 828 | 8.037 | −12.521 | 16.166 | 1.00 | 9.67 |
| ATOM | 2369 | CG | LEU | A | 828 | 7.315 | −11.431 | 16.966 | 1.00 | 14.41 |
| ATOM | 2370 | CD1 | LEU | A | 828 | 6.653 | −12.048 | 18.185 | 1.00 | 16.13 |
| ATOM | 2371 | CD2 | LEU | A | 828 | 8.300 | −10.351 | 17.389 | 1.00 | 14.03 |
| ATOM | 2372 | C | LEU | A | 828 | 5.902 | −13.218 | 15.036 | 1.00 | 11.55 |
| ATOM | 2373 | O | LEU | A | 828 | 4.814 | −13.290 | 15.609 | 1.00 | 12.50 |
| ATOM | 2374 | N | TYR | A | 829 | 6.024 | −12.717 | 13.812 | 1.00 | 10.38 |
| ATOM | 2375 | CA | TYR | A | 829 | 4.851 | −12.217 | 13.109 | 1.00 | 11.38 |
| ATOM | 2376 | CB | TYR | A | 829 | 5.286 | −11.392 | 11.892 | 1.00 | 9.07 |
| ATOM | 2377 | CG | TYR | A | 829 | 5.967 | −10.112 | 12.324 | 1.00 | 13.32 |
| ATOM | 2378 | CD1 | TYR | A | 829 | 5.298 | −9.189 | 13.118 | 1.00 | 8.62 |
| ATOM | 2379 | CE1 | TYR | A | 829 | 5.926 | −8.037 | 13.569 | 1.00 | 10.58 |
| ATOM | 2380 | CD2 | TYR | A | 829 | 7.290 | −9.849 | 11.989 | 1.00 | 10.78 |
| ATOM | 2381 | CE2 | TYR | A | 829 | 7.930 | −8.699 | 12.440 | 1.00 | 8.02 |
| ATOM | 2382 | CZ | TYR | A | 829 | 7.241 | −7.800 | 13.228 | 1.00 | 8.64 |
| ATOM | 2383 | OH | TYR | A | 829 | 7.867 | −6.659 | 13.688 | 1.00 | 10.63 |
| ATOM | 2384 | C | TYR | A | 829 | 3.908 | −13.349 | 12.731 | 1.00 | 12.07 |
| ATOM | 2385 | O | TYR | A | 829 | 2.704 | −13.140 | 12.594 | 1.00 | 14.07 |
| ATOM | 2386 | N | GLU | A | 830 | 4.452 | −14.555 | 12.585 | 1.00 | 14.19 |
| ATOM | 2387 | CA | GLU | A | 830 | 3.614 | −15.709 | 12.274 | 1.00 | 14.53 |
| ATOM | 2388 | CB | GLU | A | 830 | 4.469 | −16.919 | 11.881 | 1.00 | 15.21 |
| ATOM | 2389 | CG | GLU | A | 830 | 5.196 | −16.751 | 10.557 | 1.00 | 18.48 |
| ATOM | 2390 | CD | GLU | A | 830 | 6.057 | −17.948 | 10.208 | 1.00 | 27.35 |
| ATOM | 2391 | OE1 | GLU | A | 830 | 6.422 | −18.701 | 11.133 | 1.00 | 25.55 |
| ATOM | 2392 | OE2 | GLU | A | 830 | 6.380 | −18.127 | 9.014 | 1.00 | 25.64 |
| ATOM | 2393 | C | GLU | A | 830 | 2.806 | −16.027 | 13.531 | 1.00 | 15.34 |
| ATOM | 2394 | O | GLU | A | 830 | 1.594 | −16.245 | 13.462 | 1.00 | 15.05 |
| ATOM | 2395 | N | ALA | A | 831 | 3.484 | −16.037 | 14.679 | 1.00 | 12.88 |
| ATOM | 2396 | CA | ALA | A | 831 | 2.836 | −16.319 | 15.958 | 1.00 | 13.60 |
| ATOM | 2397 | CB | ALA | A | 831 | 3.877 | −16.341 | 17.080 | 1.00 | 14.75 |
| ATOM | 2398 | C | ALA | A | 831 | 1.764 | −15.279 | 16.264 | 1.00 | 14.36 |
| ATOM | 2399 | O | ALA | A | 831 | 0.666 | −15.617 | 16.714 | 1.00 | 13.70 |
| ATOM | 2400 | N | LEU | A | 832 | 2.085 | −14.010 | 16.026 | 1.00 | 11.70 |
| ATOM | 2401 | CA | LEU | A | 832 | 1.135 | −12.935 | 16.276 | 1.00 | 12.90 |
| ATOM | 2402 | CB | LEU | A | 832 | 1.800 | −11.577 | 16.030 | 1.00 | 12.02 |
| ATOM | 2403 | CG | LEU | A | 832 | 0.935 | −10.334 | 16.256 | 1.00 | 13.07 |
| ATOM | 2404 | CD1 | LEU | A | 832 | 0.367 | −10.354 | 17.670 | 1.00 | 16.97 |
| ATOM | 2405 | CD2 | LEU | A | 832 | 1.764 | −9.076 | 16.018 | 1.00 | 12.70 |
| ATOM | 2406 | C | LEU | A | 832 | −0.093 | −13.098 | 15.382 | 1.00 | 13.07 |
| ATOM | 2407 | O | LEU | A | 832 | −1.221 | −12.851 | 15.811 | 1.00 | 12.63 |
| ATOM | 2408 | N | THR | A | 833 | 0.125 | −13.522 | 14.140 | 1.00 | 10.33 |
| ATOM | 2409 | CA | THR | A | 833 | −0.985 | −13.720 | 13.213 | 1.00 | 11.65 |
| ATOM | 2410 | CB | THR | A | 833 | −0.471 | −14.019 | 11.798 | 1.00 | 15.54 |
| ATOM | 2411 | OG1 | THR | A | 833 | 0.250 | −12.881 | 11.309 | 1.00 | 13.44 |
| ATOM | 2412 | CG2 | THR | A | 833 | −1.631 | −14.319 | 10.856 | 1.00 | 20.09 |
| ATOM | 2413 | C | THR | A | 833 | −1.856 | −14.875 | 13.709 | 1.00 | 13.24 |
| ATOM | 2414 | O | THR | A | 833 | −3.070 | −14.879 | 13.512 | 1.00 | 14.62 |
| ATOM | 2415 | N | HIS | A | 834 | −1.233 | −15.855 | 14.358 | 1.00 | 12.04 |
| ATOM | 2416 | CA | HIS | A | 834 | −1.974 | −16.988 | 14.901 | 1.00 | 15.35 |
| ATOM | 2417 | CB | HIS | A | 834 | −1.010 | −18.045 | 15.451 | 1.00 | 17.31 |
| ATOM | 2418 | CG | HIS | A | 834 | −1.686 | −19.161 | 16.174 | 1.00 | 21.70 |
| ATOM | 2419 | CD2 | HIS | A | 834 | −2.005 | −20.422 | 15.792 | 1.00 | 27.35 |
| ATOM | 2420 | ND1 | HIS | A | 834 | −2.158 | −19.040 | 17.471 | 1.00 | 27.23 |
| ATOM | 2421 | CE1 | HIS | A | 834 | −2.728 | −20.162 | 17.843 | 1.00 | 22.73 |
| ATOM | 2422 | NE2 | HIS | A | 834 | −2.650 | −21.026 | 16.836 | 1.00 | 26.03 |
| ATOM | 2423 | C | HIS | A | 834 | −2.907 | −16.495 | 16.011 | 1.00 | 16.43 |
| ATOM | 2424 | O | HIS | A | 834 | −4.061 | −16.917 | 16.101 | 1.00 | 16.32 |
| ATOM | 2425 | N | VAL | A | 835 | −2.402 | −15.600 | 16.852 | 1.00 | 14.39 |
| ATOM | 2426 | CA | VAL | A | 835 | −3.201 | −15.042 | 17.940 | 1.00 | 16.17 |
| ATOM | 2427 | CB | VAL | A | 835 | −2.329 | −14.177 | 18.879 | 1.00 | 20.06 |
| ATOM | 2428 | CG1 | VAL | A | 835 | −3.203 | −13.433 | 19.878 | 1.00 | 26.18 |
| ATOM | 2429 | CG2 | VAL | A | 835 | −1.336 | −15.067 | 19.616 | 1.00 | 22.85 |
| ATOM | 2430 | C | VAL | A | 835 | −4.345 | −14.196 | 17.376 | 1.00 | 18.30 |
| ATOM | 2431 | O | VAL | A | 835 | −5.462 | −14.219 | 17.899 | 1.00 | 16.68 |
| ATOM | 2432 | N | SER | A | 836 | −4.061 | −13.461 | 16.302 | 1.00 | 15.55 |
| ATOM | 2433 | CA | SER | A | 836 | −5.060 | −12.620 | 15.646 | 1.00 | 15.62 |
| ATOM | 2434 | CB | SER | A | 836 | −5.081 | −11.222 | 16.267 | 1.00 | 18.44 |
| ATOM | 2435 | OG | SER | A | 836 | −6.092 | −10.430 | 15.665 | 1.00 | 24.05 |
| ATOM | 2436 | C | SER | A | 836 | −4.744 | −12.516 | 14.153 | 1.00 | 15.03 |
| ATOM | 2437 | O | SER | A | 836 | −3.754 | −11.901 | 13.759 | 1.00 | 14.82 |
| ATOM | 2438 | N | GLU | A | 837 | −5.596 | −13.123 | 13.334 | 1.00 | 16.29 |
| ATOM | 2439 | CA | GLU | A | 837 | −5.423 | −13.139 | 11.886 | 1.00 | 16.40 |
| ATOM | 2440 | CB | GLU | A | 837 | −6.578 | −13.922 | 11.244 | 1.00 | 19.29 |
| ATOM | 2441 | CG | GLU | A | 837 | −6.524 | −14.017 | 9.722 | 1.00 | 30.56 |
| ATOM | 2442 | CD | GLU | A | 837 | −5.259 | −14.685 | 9.213 | 1.00 | 40.78 |
| ATOM | 2443 | OE1 | GLU | A | 837 | −4.925 | −15.785 | 9.704 | 1.00 | 33.63 |
| ATOM | 2444 | OE2 | GLU | A | 837 | −4.603 | −14.112 | 8.316 | 1.00 | 45.55 |
| ATOM | 2445 | C | GLU | A | 837 | −5.324 | −11.749 | 11.256 | 1.00 | 14.00 |
| ATOM | 2446 | O | GLU | A | 837 | −4.672 | −11.574 | 10.227 | 1.00 | 16.56 |
| ATOM | 2447 | N | ASP | A | 838 | −5.967 | −10.762 | 11.871 | 1.00 | 14.38 |
| ATOM | 2448 | CA | ASP | A | 838 | −5.938 | −9.402 | 11.349 | 1.00 | 12.78 |
| ATOM | 2449 | CB | ASP | A | 838 | −6.990 | −8.547 | 12.060 | 1.00 | 12.80 |
| ATOM | 2450 | CG | ASP | A | 838 | −8.410 | −8.929 | 11.669 | 1.00 | 31.66 |
| ATOM | 2451 | OD1 | ASP | A | 838 | −8.770 | −8.750 | 10.487 | 1.00 | 25.15 |
| ATOM | 2452 | OD2 | ASP | A | 838 | −9.162 | −9.414 | 12.540 | 1.00 | 29.94 |
| ATOM | 2453 | C | ASP | A | 838 | −4.558 | −8.751 | 11.468 | 1.00 | 11.81 |
| ATOM | 2454 | O | ASP | A | 838 | −4.355 | −7.625 | 11.017 | 1.00 | 12.87 |
| ATOM | 2455 | N | CYS | A | 839 | −3.610 | −9.456 | 12.080 | 1.00 | 14.34 |
| ATOM | 2456 | CA | CYS | A | 839 | −2.254 | −8.929 | 12.203 | 1.00 | 14.23 |
| ATOM | 2457 | CB | CYS | A | 839 | −1.619 | −9.354 | 13.533 | 1.00 | 12.27 |
| ATOM | 2458 | SG | CYS | A | 839 | −2.115 | −8.349 | 14.969 | 1.00 | 12.53 |
| ATOM | 2459 | C | CYS | A | 839 | −1.412 | −9.440 | 11.038 | 1.00 | 15.74 |
| ATOM | 2460 | O | CYS | A | 839 | −0.222 | −9.142 | 10.941 | 1.00 | 13.52 |
| ATOM | 2461 | N | PHE | A | 840 | −2.044 | −10.209 | 10.152 | 1.00 | 14.15 |
| ATOM | 2462 | CA | PHE | A | 840 | −1.364 | −10.765 | 8.983 | 1.00 | 14.85 |
| ATOM | 2463 | CB | PHE | A | 840 | −2.370 | −11.451 | 8.052 | 1.00 | 18.01 |
| ATOM | 2464 | CG | PHE | A | 840 | −1.760 | −11.926 | 6.764 | 1.00 | 16.92 |
| ATOM | 2465 | CD1 | PHE | A | 840 | −0.886 | −13.001 | 6.751 | 1.00 | 13.52 |
| ATOM | 2466 | CD2 | PHE | A | 840 | −2.010 | −11.257 | 5.575 | 1.00 | 19.38 |
| ATOM | 2467 | CE1 | PHE | A | 840 | −0.269 | −13.398 | 5.575 | 1.00 | 23.30 |
| ATOM | 2468 | CE2 | PHE | A | 840 | −1.397 | −11.649 | 4.396 | 1.00 | 23.75 |
| ATOM | 2469 | CZ | PHE | A | 840 | −0.525 | −12.720 | 4.397 | 1.00 | 22.76 |
| ATOM | 2470 | C | PHE | A | 840 | −0.541 | −9.768 | 8.155 | 1.00 | 13.70 |
| ATOM | 2471 | O | PHE | A | 840 | 0.546 | −10.105 | 7.678 | 1.00 | 13.33 |
| ATOM | 2472 | N | PRO | A | 841 | −1.049 | −8.537 | 7.958 | 1.00 | 12.97 |
| ATOM | 2473 | CD | PRO | A | 841 | −2.369 | −8.002 | 8.339 | 1.00 | 14.47 |
| ATOM | 2474 | CA | PRO | A | 841 | −0.294 | −7.557 | 7.168 | 1.00 | 12.84 |
| ATOM | 2475 | CB | PRO | A | 841 | −1.127 | −6.287 | 7.312 | 1.00 | 14.37 |
| ATOM | 2476 | CG | PRO | A | 841 | −2.533 | −6.826 | 7.391 | 1.00 | 14.58 |
| ATOM | 2477 | C | PRO | A | 841 | 1.151 | −7.367 | 7.630 | 1.00 | 14.54 |
| ATOM | 2478 | O | PRO | A | 841 | 2.034 | −7.065 | 6.826 | 1.00 | 13.75 |
| ATOM | 2479 | N | LEU | A | 842 | 1.393 | −7.530 | 8.926 | 1.00 | 11.66 |
| ATOM | 2480 | CA | LEU | A | 842 | 2.747 | −7.384 | 9.444 | 1.00 | 12.35 |
| ATOM | 2481 | CB | LEU | A | 842 | 2.732 | −7.430 | 10.977 | 1.00 | 11.85 |
| ATOM | 2482 | CG | LEU | A | 842 | 2.039 | −6.228 | 11.624 | 1.00 | 12.78 |
| ATOM | 2483 | CD1 | LEU | A | 842 | 1.900 | −6.434 | 13.131 | 1.00 | 14.23 |
| ATOM | 2484 | CD2 | LEU | A | 842 | 2.837 | −4.975 | 11.321 | 1.00 | 14.25 |
| ATOM | 2485 | C | LEU | A | 842 | 3.613 | −8.513 | 8.880 | 1.00 | 13.95 |
| ATOM | 2486 | O | LEU | A | 842 | 4.741 | −8.290 | 8.444 | 1.00 | 13.84 |
| ATOM | 2487 | N | LEU | A | 843 | 3.067 | −9.727 | 8.882 | 1.00 | 11.90 |
| ATOM | 2488 | CA | LEU | A | 843 | 3.779 | −10.889 | 8.359 | 1.00 | 11.64 |
| ATOM | 2489 | CB | LEU | A | 843 | 2.995 | −12.161 | 8.670 | 1.00 | 10.07 |
| ATOM | 2490 | CG | LEU | A | 843 | 3.532 | −13.468 | 8.084 | 1.00 | 9.50 |
| ATOM | 2491 | CD1 | LEU | A | 843 | 4.940 | −13.735 | 8.588 | 1.00 | 13.64 |
| ATOM | 2492 | CD2 | LEU | A | 843 | 2.597 | −14.608 | 8.466 | 1.00 | 12.37 |
| ATOM | 2493 | C | LEU | A | 843 | 3.971 | −10.751 | 6.847 | 1.00 | 11.74 |
| ATOM | 2494 | O | LEU | A | 843 | 5.054 | −11.003 | 6.319 | 1.00 | 13.14 |
| ATOM | 2495 | N | ASP | A | 844 | 2.909 | −10.342 | 6.160 | 1.00 | 13.31 |
| ATOM | 2496 | CA | ASP | A | 844 | 2.943 | −10.154 | 4.711 | 1.00 | 14.45 |
| ATOM | 2497 | CB | ASP | A | 844 | 1.573 | −9.639 | 4.245 | 1.00 | 18.19 |
| ATOM | 2498 | CG | ASP | A | 844 | 1.354 | −9.771 | 2.739 | 1.00 | 13.51 |
| ATOM | 2499 | OD1 | ASP | A | 844 | 2.141 | −10.459 | 2.057 | 1.00 | 18.86 |
| ATOM | 2500 | OD2 | ASP | A | 844 | 0.367 | −9.187 | 2.243 | 1.00 | 17.91 |
| ATOM | 2501 | C | ASP | A | 844 | 4.053 | −9.153 | 4.366 | 1.00 | 13.87 |
| ATOM | 2502 | O | ASP | A | 844 | 4.854 | −9.386 | 3.460 | 1.00 | 13.07 |
| ATOM | 2503 | N | GLY | A | 845 | 4.100 | −8.046 | 5.104 | 1.00 | 12.67 |
| ATOM | 2504 | CA | GLY | A | 845 | 5.115 | −7.035 | 4.862 | 1.00 | 11.69 |
| ATOM | 2505 | C | GLY | A | 845 | 6.529 | −7.540 | 5.097 | 1.00 | 11.05 |
| ATOM | 2506 | O | GLY | A | 845 | 7.447 | −7.240 | 4.330 | 1.00 | 12.51 |
| ATOM | 2507 | N | CYS | A | 846 | 6.704 | −8.314 | 6.161 | 1.00 | 10.28 |
| ATOM | 2508 | CA | CYS | A | 846 | 8.005 | −8.882 | 6.507 | 1.00 | 10.93 |
| ATOM | 2509 | CB | CYS | A | 846 | 7.889 | −9.643 | 7.831 | 1.00 | 14.58 |
| ATOM | 2510 | SG | CYS | A | 846 | 9.443 | −10.273 | 8.485 | 1.00 | 12.82 |
| ATOM | 2511 | C | CYS | A | 846 | 8.497 | −9.821 | 5.398 | 1.00 | 11.92 |
| ATOM | 2512 | O | CYS | A | 846 | 9.661 | −9.762 | 4.985 | 1.00 | 11.24 |
| ATOM | 2513 | N | ARG | A | 847 | 7.605 | −10.683 | 4.914 | 1.00 | 12.77 |
| ATOM | 2514 | CA | ARG | A | 847 | 7.953 | −11.618 | 3.853 | 1.00 | 13.72 |
| ATOM | 2515 | CB | ARG | A | 847 | 6.762 | −12.527 | 3.535 | 1.00 | 11.63 |
| ATOM | 2516 | CG | ARG | A | 847 | 6.457 | −13.545 | 4.619 | 1.00 | 13.10 |
| ATOM | 2517 | CD | ARG | A | 847 | 5.180 | −14.308 | 4.316 | 1.00 | 17.35 |
| ATOM | 2518 | NE | ARG | A | 847 | 4.978 | −15.402 | 5.262 | 1.00 | 19.23 |
| ATOM | 2519 | CZ | ARG | A | 847 | 3.840 | −16.077 | 5.389 | 1.00 | 21.47 |
| ATOM | 2520 | NH1 | ARG | A | 847 | 2.796 | −15.769 | 4.632 | 1.00 | 24.29 |
| ATOM | 2521 | NH2 | ARG | A | 847 | 3.750 | −17.059 | 6.275 | 1.00 | 27.21 |
| ATOM | 2522 | C | ARG | A | 847 | 8.371 | −10.870 | 2.592 | 1.00 | 13.53 |
| ATOM | 2523 | O | ARG | A | 847 | 9.320 | −11.265 | 1.906 | 1.00 | 13.20 |
| ATOM | 2524 | N | LYS | A | 848 | 7.659 | −9.793 | 2.283 | 1.00 | 12.35 |
| ATOM | 2525 | CA | LYS | A | 848 | 7.977 | −9.007 | 1.099 | 1.00 | 14.65 |
| ATOM | 2526 | CB | LYS | A | 848 | 6.925 | −7.915 | 0.880 | 1.00 | 16.22 |
| ATOM | 2527 | CG | LYS | A | 848 | 5.567 | −8.447 | 0.413 | 1.00 | 14.77 |
| ATOM | 2528 | CD | LYS | A | 848 | 4.579 | −7.308 | 0.195 | 1.00 | 19.65 |
| ATOM | 2529 | CE | LYS | A | 848 | 3.210 | −7.817 | −0.206 | 1.00 | 19.42 |
| ATOM | 2530 | NZ | LYS | A | 848 | 2.214 | −6.709 | −0.212 | 1.00 | 23.11 |
| ATOM | 2531 | C | LYS | A | 848 | 9.364 | −8.392 | 1.229 | 1.00 | 14.80 |
| ATOM | 2532 | O | LYS | A | 848 | 10.112 | −8.319 | 0.253 | 1.00 | 13.12 |
| ATOM | 2533 | N | ASN | A | 849 | 9.713 | −7.953 | 2.435 | 1.00 | 13.42 |
| ATOM | 2534 | CA | ASN | A | 849 | 11.034 | −7.369 | 2.648 | 1.00 | 13.07 |
| ATOM | 2535 | CB | ASN | A | 849 | 11.086 | −6.646 | 3.995 | 1.00 | 13.67 |
| ATOM | 2536 | CG | ASN | A | 849 | 10.412 | −5.288 | 3.950 | 1.00 | 17.85 |
| ATOM | 2537 | OD1 | ASN | A | 849 | 9.863 | −4.821 | 4.949 | 1.00 | 16.87 |
| ATOM | 2538 | ND2 | ASN | A | 849 | 10.461 | −4.638 | 2.790 | 1.00 | 13.21 |
| ATOM | 2539 | C | ASN | A | 849 | 12.122 | −8.434 | 2.568 | 1.00 | 12.00 |
| ATOM | 2540 | O | ASN | A | 849 | 13.252 | −8.148 | 2.165 | 1.00 | 11.88 |
| ATOM | 2541 | N | ARG | A | 850 | 11.797 | −9.667 | 2.945 | 1.00 | 11.53 |
| ATOM | 2542 | CA | ARG | A | 850 | 12.793 | −10.729 | 2.860 | 1.00 | 11.98 |
| ATOM | 2543 | CB | ARG | A | 850 | 12.285 | −12.037 | 3.473 | 1.00 | 14.13 |
| ATOM | 2544 | CG | ARG | A | 850 | 13.373 | −13.110 | 3.508 | 1.00 | 13.55 |
| ATOM | 2545 | CD | ARG | A | 850 | 12.838 | −14.511 | 3.781 | 1.00 | 18.02 |
| ATOM | 2546 | NE | ARG | A | 850 | 13.930 | −15.457 | 4.001 | 1.00 | 16.95 |
| ATOM | 2547 | CZ | ARG | A | 850 | 14.743 | −15.919 | 3.052 | 1.00 | 14.40 |
| ATOM | 2548 | NH1 | ARG | A | 850 | 14.595 | −15.533 | 1.790 | 1.00 | 16.03 |
| ATOM | 2549 | NH2 | ARG | A | 850 | 15.727 | −16.754 | 3.373 | 1.00 | 12.51 |
| ATOM | 2550 | C | ARG | A | 850 | 13.138 | −10.972 | 1.399 | 1.00 | 13.81 |
| ATOM | 2551 | O | ARG | A | 850 | 14.304 | −11.151 | 1.054 | 1.00 | 15.14 |
| ATOM | 2552 | N | GLN | A | 851 | 12.118 | −10.985 | 0.542 | 1.00 | 16.03 |
| ATOM | 2553 | CA | GLN | A | 851 | 12.317 | −11.193 | −0.892 | 1.00 | 15.42 |
| ATOM | 2554 | CB | GLN | A | 851 | 10.969 | −11.156 | −1.622 | 1.00 | 17.30 |
| ATOM | 2555 | CG | GLN | A | 851 | 11.039 | −11.399 | −3.127 | 1.00 | 30.46 |
| ATOM | 2556 | CD | GLN | A | 851 | 11.475 | −10.174 | −3.912 | 1.00 | 34.81 |
| ATOM | 2557 | OE1 | GLN | A | 851 | 10.862 | −9.110 | −3.814 | 1.00 | 37.41 |
| ATOM | 2558 | NE2 | GLN | A | 851 | 12.534 | −10.320 | −4.704 | 1.00 | 40.89 |
| ATOM | 2559 | C | GLN | A | 851 | 13.229 | −10.109 | −1.436 | 1.00 | 16.49 |
| ATOM | 2560 | O | GLN | A | 851 | 14.151 | −10.371 | −2.208 | 1.00 | 15.70 |
| ATOM | 2561 | N | LYS | A | 852 | 12.953 | −8.867 | −1.014 | 1.00 | 15.86 |
| ATOM | 2562 | CA | LYS | A | 852 | 13.705 | −7.685 | −1.428 | 1.00 | 16.63 |
| ATOM | 2563 | CB | LYS | A | 852 | 13.065 | −6.405 | −0.838 | 1.00 | 13.58 |
| ATOM | 2564 | CG | LYS | A | 852 | 11.802 | −6.008 | −1.561 | 1.00 | 18.37 |
| ATOM | 2565 | CD | LYS | A | 852 | 12.183 | −5.582 | −2.958 | 1.00 | 31.95 |
| ATOM | 2566 | CE | LYS | A | 852 | 11.002 | −5.087 | −3.751 | 1.00 | 37.83 |
| ATOM | 2567 | NZ | LYS | A | 852 | 11.421 | −4.599 | −5.099 | 1.00 | 41.92 |
| ATOM | 2568 | C | LYS | A | 852 | 15.175 | −7.754 | −1.037 | 1.00 | 15.46 |
| ATOM | 2569 | O | LYS | A | 852 | 16.055 | −7.542 | −1.874 | 1.00 | 14.58 |
| ATOM | 2570 | N | TRP | A | 853 | 15.446 | −8.063 | 0.225 | 1.00 | 12.99 |
| ATOM | 2571 | CA | TRP | A | 853 | 16.821 | −8.168 | 0.705 | 1.00 | 12.72 |
| ATOM | 2572 | CB | TRP | A | 853 | 16.843 | −8.299 | 2.228 | 1.00 | 11.94 |
| ATOM | 2573 | CG | TRP | A | 853 | 16.640 | −7.011 | 2.957 | 1.00 | 11.40 |
| ATOM | 2574 | CD2 | TRP | A | 853 | 17.485 | −5.864 | 2.913 | 1.00 | 14.74 |
| ATOM | 2575 | CE2 | TRP | A | 853 | 16.929 | −4.888 | 3.763 | 1.00 | 12.41 |
| ATOM | 2576 | CE3 | TRP | A | 853 | 18.675 | −5.554 | 2.236 | 1.00 | 10.90 |
| ATOM | 2577 | CD1 | TRP | A | 853 | 15.618 | −6.701 | 3.813 | 1.00 | 11.02 |
| ATOM | 2578 | NE1 | TRP | A | 853 | 15.781 | −5.429 | 4.303 | 1.00 | 14.15 |
| ATOM | 2579 | CZ2 | TRP | A | 853 | 17.502 | −3.638 | 3.958 | 1.00 | 12.03 |
| ATOM | 2580 | CZ3 | TRP | A | 853 | 19.246 | −4.312 | 2.427 | 1.00 | 13.77 |
| ATOM | 2581 | CH2 | TRP | A | 853 | 18.663 | −3.370 | 3.280 | 1.00 | 16.08 |
| ATOM | 2582 | C | TRP | A | 853 | 17.543 | −9.357 | 0.074 | 1.00 | 13.55 |
| ATOM | 2583 | O | TRP | A | 853 | 18.722 | −9.265 | −0.282 | 1.00 | 13.19 |
| ATOM | 2584 | N | GLN | A | 854 | 16.836 | −10.472 | −0.062 | 1.00 | 12.06 |
| ATOM | 2585 | CA | GLN | A | 854 | 17.427 | −11.667 | −0.657 | 1.00 | 13.49 |
| ATOM | 2586 | CB | GLN | A | 854 | 16.426 | −12.822 | −0.630 | 1.00 | 13.86 |
| ATOM | 2587 | CG | GLN | A | 854 | 16.934 | −14.128 | −1.242 | 1.00 | 15.91 |
| ATOM | 2588 | CD | GLN | A | 854 | 18.267 | −14.585 | −0.665 | 1.00 | 23.66 |
| ATOM | 2589 | OE1 | GLN | A | 854 | 18.544 | −14.404 | 0.520 | 1.00 | 20.44 |
| ATOM | 2590 | NE2 | GLN | A | 854 | 19.091 | −15.199 | −1.504 | 1.00 | 26.84 |
| ATOM | 2591 | C | GLN | A | 854 | 17.869 | −11.400 | −2.094 | 1.00 | 13.14 |
| ATOM | 2592 | O | GLN | A | 854 | 18.920 | −11.880 | −2.524 | 1.00 | 15.55 |
| ATOM | 2593 | N | ALA | A | 855 | 17.069 | −10.635 | −2.832 | 1.00 | 14.63 |
| ATOM | 2594 | CA | ALA | A | 855 | 17.408 | −10.318 | −4.219 | 1.00 | 15.08 |
| ATOM | 2595 | CB | ALA | A | 855 | 16.264 | −9.563 | −4.884 | 1.00 | 17.06 |
| ATOM | 2596 | C | ALA | A | 855 | 18.697 | −9.504 | −4.303 | 1.00 | 16.23 |
| ATOM | 2597 | O | ALA | A | 855 | 19.439 | −9.611 | −5.278 | 1.00 | 17.06 |
| ATOM | 2598 | N | LEU | A | 856 | 18.959 | −8.689 | −3.287 | 1.00 | 15.11 |
| ATOM | 2599 | CA | LEU | A | 856 | 20.178 | −7.889 | −3.261 | 1.00 | 17.23 |
| ATOM | 2600 | CB | LEU | A | 856 | 20.031 | −6.715 | −2.284 | 1.00 | 11.53 |
| ATOM | 2601 | CG | LEU | A | 856 | 18.968 | −5.676 | −2.643 | 1.00 | 15.60 |
| ATOM | 2602 | CD1 | LEU | A | 856 | 18.996 | −4.549 | −1.634 | 1.00 | 19.73 |
| ATOM | 2603 | CD2 | LEU | A | 856 | 19.226 | −5.145 | −4.047 | 1.00 | 12.95 |
| ATOM | 2604 | C | LEU | A | 856 | 21.362 | −8.754 | −2.835 | 1.00 | 17.35 |
| ATOM | 2605 | O | LEU | A | 856 | 22.478 | −8.601 | −3.337 | 1.00 | 17.30 |
| ATOM | 2606 | N | ALA | A | 857 | 21.109 | −9.674 | −1.911 | 1.00 | 16.52 |
| ATOM | 2607 | CA | ALA | A | 857 | 22.156 | −10.545 | −1.396 | 1.00 | 17.82 |
| ATOM | 2608 | CB | ALA | A | 857 | 21.708 | −11.155 | −0.079 | 1.00 | 15.78 |
| ATOM | 2609 | C | ALA | A | 857 | 22.616 | −11.651 | −2.339 | 1.00 | 19.95 |
| ATOM | 2610 | O | ALA | A | 857 | 23.781 | −12.048 | −2.305 | 1.00 | 21.30 |
| ATOM | 2611 | N | GLU | A | 858 | 21.715 | −12.144 | −3.181 | 1.00 | 19.75 |
| ATOM | 2612 | CA | GLU | A | 858 | 22.059 | −13.233 | −4.091 | 1.00 | 23.14 |
| ATOM | 2613 | CB | GLU | A | 858 | 20.798 | −14.008 | −4.475 | 1.00 | 28.23 |
| ATOM | 2614 | CG | GLU | A | 858 | 19.735 | −13.155 | −5.139 | 1.00 | 35.46 |
| ATOM | 2615 | CD | GLU | A | 858 | 18.516 | −13.957 | −5.548 | 1.00 | 41.30 |
| ATOM | 2616 | OE1 | GLU | A | 858 | 17.928 | −14.635 | −4.679 | 1.00 | 46.06 |
| ATOM | 2617 | OE2 | GLU | A | 858 | 18.144 | −13.905 | −6.739 | 1.00 | 53.40 |
| ATOM | 2618 | C | GLU | A | 858 | 22.773 | −12.775 | −5.356 | 1.00 | 24.66 |
| ATOM | 2619 | O | GLU | A | 858 | 22.769 | −11.561 | −5.640 | 1.00 | 27.06 |
| ATOM | 2620 | OXT | GLU | A | 858 | 23.319 | −13.654 | −6.053 | 1.00 | 33.90 |
| TER | 2621 | | GLU | A | 858 | | | | | |
| ATOM | 2622 | O | HOH | W | 1 | 14.659 | 12.373 | 18.930 | 1.00 | 11.90 |
| ATOM | 2623 | O | HOH | W | 2 | 9.029 | 0.616 | 15.467 | 1.00 | 15.60 |
| ATOM | 2624 | O | HOH | W | 3 | 17.339 | −0.310 | 24.269 | 1.00 | 9.87 |
| ATOM | 2625 | O | HOH | W | 4 | 17.787 | 8.492 | 22.312 | 1.00 | 9.91 |
| ATOM | 2626 | O | HOH | W | 5 | 22.322 | 0.101 | 41.405 | 1.00 | 10.58 |
| ATOM | 2627 | O | HOH | W | 6 | 7.037 | −4.929 | 2.938 | 1.00 | 15.52 |
| ATOM | 2628 | O | HOH | W | 7 | 24.844 | 0.942 | 17.890 | 1.00 | 12.19 |
| ATOM | 2629 | O | HOH | W | 8 | 17.812 | −5.285 | 21.940 | 1.00 | 13.09 |
| ATOM | 2630 | O | HOH | W | 9 | 19.355 | −1.244 | 21.749 | 1.00 | 11.09 |
| ATOM | 2631 | O | HOH | W | 10 | 11.998 | −15.929 | 13.123 | 1.00 | 15.08 |
| ATOM | 2632 | O | HOH | W | 11 | 17.595 | 6.868 | 5.858 | 1.00 | 17.40 |
| ATOM | 2633 | O | HOH | W | 12 | 20.522 | −8.124 | 28.490 | 1.00 | 16.30 |
| ATOM | 2634 | O | HOH | W | 13 | 7.122 | 8.426 | 30.706 | 1.00 | 12.74 |
| ATOM | 2635 | O | HOH | W | 14 | 14.281 | −0.736 | 24.882 | 1.00 | 10.20 |
| ATOM | 2636 | O | HOH | W | 15 | 27.778 | −4.812 | 7.714 | 1.00 | 14.34 |
| ATOM | 2637 | O | HOH | W | 16 | 45.697 | −5.856 | 19.373 | 1.00 | 13.79 |
| ATOM | 2638 | O | HOH | W | 17 | 32.028 | 0.851 | 11.859 | 1.00 | 12.49 |
| ATOM | 2639 | O | HOH | W | 18 | 12.716 | −7.833 | 22.260 | 1.00 | 11.62 |
| ATOM | 2640 | O | HOH | W | 19 | 20.770 | −7.019 | 24.953 | 1.00 | 13.66 |
| ATOM | 2641 | O | HOH | W | 20 | 9.679 | −8.387 | 30.457 | 1.00 | 18.04 |
| ATOM | 2642 | O | HOH | W | 21 | 20.996 | −1.566 | 19.584 | 1.00 | 15.27 |
| ATOM | 2643 | O | HOH | W | 22 | 5.367 | −8.570 | 29.548 | 1.00 | 17.36 |
| ATOM | 2644 | O | HOH | W | 23 | 25.138 | −9.337 | 1.725 | 1.00 | 17.63 |
| ATOM | 2645 | O | HOH | W | 24 | −9.020 | −0.116 | 43.708 | 1.00 | 15.91 |
| ATOM | 2646 | O | HOH | W | 25 | 22.657 | −6.174 | 29.194 | 1.00 | 17.18 |
| ATOM | 2647 | O | HOH | W | 26 | −4.280 | 19.557 | 21.192 | 1.00 | 19.85 |
| ATOM | 2648 | O | HOH | W | 27 | 14.289 | 6.604 | 13.852 | 1.00 | 14.85 |
| ATOM | 2649 | O | HOH | W | 28 | 11.411 | −1.302 | 13.935 | 1.00 | 12.80 |
| ATOM | 2650 | O | HOH | W | 29 | 11.700 | 13.882 | 15.200 | 1.00 | 14.97 |
| ATOM | 2651 | O | HOH | W | 30 | 18.410 | 10.076 | 12.196 | 1.00 | 19.59 |
| ATOM | 2652 | O | HOH | W | 31 | 26.709 | 7.571 | 23.378 | 1.00 | 17.44 |
| ATOM | 2653 | O | HOH | W | 32 | 25.420 | 5.805 | 17.773 | 1.00 | 17.40 |
| ATOM | 2654 | O | HOH | W | 33 | 11.087 | 10.589 | 10.070 | 1.00 | 17.81 |
| ATOM | 2655 | O | HOH | W | 34 | 11.500 | −19.954 | 26.231 | 1.00 | 16.07 |
| ATOM | 2656 | O | HOH | W | 35 | −5.204 | −5.042 | 34.106 | 1.00 | 17.24 |
| ATOM | 2657 | O | HOH | W | 36 | 1.941 | 9.940 | 36.428 | 1.00 | 13.95 |
| ATOM | 2658 | O | HOH | W | 37 | 23.916 | 7.238 | 15.924 | 1.00 | 14.00 |
| ATOM | 2659 | O | HOH | W | 38 | 4.522 | 21.473 | 19.911 | 1.00 | 20.60 |
| ATOM | 2660 | O | HOH | W | 39 | 7.654 | 32.374 | 24.586 | 1.00 | 19.77 |
| ATOM | 2661 | O | HOH | W | 40 | −11.105 | 8.617 | 12.732 | 1.00 | 20.19 |
| ATOM | 2662 | O | HOH | W | 41 | 14.266 | 14.289 | 20.855 | 1.00 | 16.50 |
| ATOM | 2663 | O | HOH | W | 42 | 27.797 | −9.140 | 13.072 | 1.00 | 17.28 |
| ATOM | 2664 | O | HOH | W | 43 | 13.361 | −5.781 | 20.636 | 1.00 | 17.62 |
| ATOM | 2665 | O | HOH | W | 44 | 9.439 | 17.952 | 22.920 | 1.00 | 20.34 |
| ATOM | 2666 | O | HOH | W | 45 | 13.506 | −19.339 | 23.851 | 1.00 | 17.51 |
| ATOM | 2667 | O | HOH | W | 46 | −9.608 | −2.484 | 44.794 | 1.00 | 18.03 |
| ATOM | 2668 | O | HOH | W | 47 | 19.179 | −3.517 | 23.479 | 1.00 | 14.63 |
| ATOM | 2669 | O | HOH | W | 48 | 20.825 | −1.771 | 7.357 | 1.00 | 15.56 |
| ATOM | 2670 | O | HOH | W | 49 | 3.953 | −2.401 | 8.103 | 1.00 | 15.98 |
| ATOM | 2671 | O | HOH | W | 50 | 5.164 | 2.828 | 12.389 | 1.00 | 17.57 |
| ATOM | 2672 | O | HOH | W | 51 | 22.475 | −6.004 | 8.390 | 1.00 | 20.31 |
| ATOM | 2673 | O | HOH | W | 52 | −1.505 | −7.713 | 3.922 | 1.00 | 20.93 |
| ATOM | 2674 | O | HOH | W | 53 | 16.030 | 13.344 | 15.066 | 1.00 | 22.86 |
| ATOM | 2675 | O | HOH | W | 54 | 3.120 | 33.114 | 17.327 | 1.00 | 18.81 |
| ATOM | 2676 | O | HOH | W | 55 | 6.517 | 19.676 | 19.112 | 1.00 | 20.43 |
| ATOM | 2677 | O | HOH | W | 56 | 27.812 | 5.941 | 11.269 | 1.00 | 19.39 |
| ATOM | 2678 | O | HOH | W | 57 | 14.704 | 9.138 | 12.638 | 1.00 | 17.37 |
| ATOM | 2679 | O | HOH | W | 58 | 10.813 | −19.129 | 28.734 | 1.00 | 23.50 |
| ATOM | 2680 | O | HOH | W | 59 | 9.603 | −13.958 | 1.403 | 1.00 | 20.05 |
| ATOM | 2681 | O | HOH | W | 60 | 46.209 | −3.550 | 17.835 | 1.00 | 18.22 |
| ATOM | 2682 | O | HOH | W | 61 | 24.760 | −2.396 | 10.006 | 1.00 | 20.80 |
| ATOM | 2683 | O | HOH | W | 62 | 11.077 | 3.087 | 12.585 | 1.00 | 21.05 |
| ATOM | 2684 | O | HOH | W | 63 | 10.534 | −10.726 | 28.998 | 1.00 | 21.18 |
| ATOM | 2685 | O | HOH | W | 64 | −0.961 | −11.064 | 33.440 | 1.00 | 18.63 |
| ATOM | 2686 | O | HOH | W | 65 | 20.890 | 0.577 | 15.388 | 1.00 | 19.77 |
| ATOM | 2687 | O | HOH | W | 66 | 21.685 | 13.866 | 20.281 | 1.00 | 24.54 |
| ATOM | 2688 | O | HOH | W | 67 | −5.012 | −3.878 | 8.180 | 1.00 | 21.24 |
| ATOM | 2689 | O | HOH | W | 68 | −10.443 | 9.371 | 15.194 | 1.00 | 19.03 |
| ATOM | 2690 | O | HOH | W | 69 | 26.635 | 8.578 | 39.152 | 1.00 | 19.48 |
| ATOM | 2691 | O | HOH | W | 70 | 21.837 | −4.044 | 23.094 | 1.00 | 14.46 |
| ATOM | 2692 | O | HOH | W | 71 | 18.979 | 0.534 | 3.275 | 1.00 | 19.32 |
| ATOM | 2693 | O | HOH | W | 72 | −7.687 | −4.529 | 35.484 | 1.00 | 19.76 |
| ATOM | 2694 | O | HOH | W | 73 | −4.441 | 9.972 | 35.483 | 1.00 | 21.75 |
| ATOM | 2695 | O | HOH | W | 74 | 22.147 | 0.611 | 18.233 | 1.00 | 21.06 |
| ATOM | 2696 | O | HOH | W | 75 | 25.625 | 7.694 | 19.834 | 1.00 | 17.79 |
| ATOM | 2697 | O | HOH | W | 76 | −9.716 | 14.941 | 11.027 | 1.00 | 20.22 |
| ATOM | 2698 | O | HOH | W | 77 | 14.015 | −1.710 | 16.866 | 1.00 | 19.03 |
| ATOM | 2699 | O | HOH | W | 78 | 21.861 | −3.953 | 20.354 | 1.00 | 17.68 |
| ATOM | 2700 | O | HOH | W | 79 | 21.854 | −11.471 | 10.659 | 1.00 | 21.05 |
| ATOM | 2701 | O | HOH | W | 80 | 15.795 | −5.944 | −4.295 | 1.00 | 17.34 |
| ATOM | 2702 | O | HOH | W | 81 | 26.427 | −5.333 | 20.748 | 1.00 | 28.65 |
| ATOM | 2703 | O | HOH | W | 82 | 0.806 | 16.637 | 36.547 | 1.00 | 22.11 |
| ATOM | 2704 | O | HOH | W | 83 | 28.883 | 5.909 | 33.300 | 1.00 | 35.99 |
| ATOM | 2705 | O | HOH | W | 84 | 0.615 | −5.836 | 36.804 | 1.00 | 21.63 |
| ATOM | 2706 | O | HOH | W | 85 | 14.416 | −12.696 | −3.692 | 1.00 | 22.36 |
| ATOM | 2707 | O | HOH | W | 86 | −4.841 | 7.644 | 43.980 | 1.00 | 19.92 |
| ATOM | 2708 | O | HOH | W | 87 | −6.059 | 5.564 | 10.293 | 1.00 | 22.49 |
| ATOM | 2709 | O | HOH | W | 88 | 7.210 | 1.031 | 13.002 | 1.00 | 19.78 |
| ATOM | 2710 | O | HOH | W | 89 | −5.786 | −6.329 | 9.023 | 1.00 | 20.32 |
| ATOM | 2711 | O | HOH | W | 90 | 19.117 | 4.909 | 35.027 | 1.00 | 17.30 |
| ATOM | 2712 | O | HOH | W | 91 | 10.592 | 13.017 | 38.293 | 1.00 | 23.66 |
| ATOM | 2713 | O | HOH | W | 92 | −9.801 | 5.234 | 16.132 | 1.00 | 20.64 |
| ATOM | 2714 | O | HOH | W | 93 | −6.370 | −17.172 | 24.540 | 1.00 | 23.65 |
| ATOM | 2715 | O | HOH | W | 94 | −6.833 | 1.342 | 45.207 | 1.00 | 17.92 |
| ATOM | 2716 | O | HOH | W | 95 | 33.715 | −7.279 | 11.000 | 1.00 | 17.67 |
| ATOM | 2717 | O | HOH | W | 96 | 5.722 | 31.659 | 29.252 | 1.00 | 19.35 |
| ATOM | 2718 | O | HOH | W | 97 | 14.155 | −2.890 | 10.083 | 1.00 | 16.30 |
| ATOM | 2719 | O | HOH | W | 98 | 19.709 | −5.898 | 20.414 | 1.00 | 27.45 |
| ATOM | 2720 | O | HOH | W | 99 | 4.418 | −3.958 | 2.629 | 1.00 | 23.70 |
| ATOM | 2721 | O | HOH | W | 100 | 5.739 | 5.419 | 41.566 | 1.00 | 26.30 |
| ATOM | 2722 | O | HOH | W | 101 | 1.882 | −10.334 | 12.547 | 1.00 | 21.56 |
| ATOM | 2723 | O | HOH | W | 102 | 1.506 | 9.558 | 7.516 | 1.00 | 21.76 |
| ATOM | 2724 | O | HOH | W | 103 | 23.211 | 1.757 | 4.698 | 1.00 | 23.21 |
| ATOM | 2725 | O | HOH | W | 104 | 36.282 | 2.363 | 9.440 | 1.00 | 27.13 |
| ATOM | 2726 | O | HOH | W | 105 | 27.600 | −11.267 | 2.472 | 1.00 | 18.59 |
| ATOM | 2727 | O | HOH | W | 106 | 16.305 | 16.757 | 22.862 | 1.00 | 25.14 |
| ATOM | 2728 | O | HOH | W | 107 | 21.223 | −0.443 | 4.807 | 1.00 | 20.51 |
| ATOM | 2729 | O | HOH | W | 108 | 12.939 | 13.840 | 12.680 | 1.00 | 26.10 |
| ATOM | 2730 | O | HOH | W | 109 | 10.191 | −9.652 | 24.549 | 1.00 | 17.97 |
| ATOM | 2731 | O | HOH | W | 110 | 9.527 | −19.990 | 11.160 | 1.00 | 34.52 |
| ATOM | 2732 | O | HOH | W | 111 | 2.300 | −4.117 | 6.982 | 1.00 | 21.40 |
| ATOM | 2733 | O | HOH | W | 112 | 31.807 | 5.599 | 25.384 | 1.00 | 19.28 |
| ATOM | 2734 | O | HOH | W | 113 | 11.858 | 5.625 | 12.858 | 1.00 | 18.80 |
| ATOM | 2735 | O | HOH | W | 114 | 2.975 | 3.851 | 3.583 | 1.00 | 28.02 |
| ATOM | 2736 | O | HOH | W | 115 | 22.328 | −3.325 | 9.278 | 1.00 | 18.13 |
| ATOM | 2737 | O | HOH | W | 116 | 7.581 | 21.278 | 17.210 | 1.00 | 26.33 |
| ATOM | 2738 | O | HOH | W | 117 | −8.959 | 18.330 | 19.702 | 1.00 | 25.41 |
| ATOM | 2739 | O | HOH | W | 118 | −7.913 | −3.625 | 46.688 | 1.00 | 16.16 |
| ATOM | 2740 | O | HOH | W | 119 | 29.369 | −4.122 | 35.016 | 1.00 | 21.35 |
| ATOM | 2741 | O | HOH | W | 120 | −10.313 | 4.301 | 39.676 | 1.00 | 20.84 |
| ATOM | 2742 | O | HOH | W | 121 | 36.288 | −8.163 | 10.383 | 1.00 | 29.41 |
| ATOM | 2743 | O | HOH | W | 122 | −5.305 | 0.154 | 7.066 | 1.00 | 32.03 |
| ATOM | 2744 | O | HOH | W | 123 | 15.892 | 9.345 | 6.948 | 1.00 | 31.98 |
| ATOM | 2745 | O | HOH | W | 124 | −7.934 | −11.981 | 26.594 | 1.00 | 24.88 |
| ATOM | 2746 | O | HOH | W | 125 | −7.700 | −14.762 | 14.417 | 1.00 | 27.16 |
| ATOM | 2747 | O | HOH | W | 126 | 7.924 | 19.345 | 32.073 | 1.00 | 27.10 |
| ATOM | 2748 | O | HOH | W | 127 | 22.963 | −15.271 | −0.194 | 1.00 | 34.57 |
| ATOM | 2749 | O | HOH | W | 128 | 14.018 | 4.418 | 0.344 | 1.00 | 27.94 |
| ATOM | 2750 | O | HOH | W | 129 | 13.334 | 16.377 | 17.301 | 1.00 | 27.05 |
| ATOM | 2751 | O | HOH | W | 130 | 30.641 | −11.391 | 0.176 | 1.00 | 21.79 |
| ATOM | 2752 | O | HOH | W | 131 | −5.143 | −9.116 | 6.397 | 1.00 | 26.22 |
| ATOM | 2753 | O | HOH | W | 132 | 44.452 | 2.118 | 22.189 | 1.00 | 25.35 |
| ATOM | 2754 | O | HOH | W | 133 | −0.942 | −8.268 | 32.526 | 1.00 | 21.73 |
| ATOM | 2755 | O | HOH | W | 134 | 32.286 | 8.100 | 17.531 | 1.00 | 30.18 |
| ATOM | 2756 | O | HOH | W | 135 | 4.471 | −22.268 | 17.254 | 1.00 | 22.18 |
| ATOM | 2757 | O | HOH | W | 136 | 20.964 | −7.154 | 10.051 | 1.00 | 23.34 |
| ATOM | 2758 | O | HOH | W | 137 | −6.984 | 26.107 | 22.746 | 1.00 | 26.33 |
| ATOM | 2759 | O | HOH | W | 138 | 17.856 | 14.348 | 24.807 | 1.00 | 20.33 |
| ATOM | 2760 | O | HOH | W | 139 | 10.038 | −5.546 | 41.193 | 1.00 | 26.62 |
| ATOM | 2761 | O | HOH | W | 140 | 13.989 | 13.837 | 16.687 | 1.00 | 27.53 |
| ATOM | 2762 | O | HOH | W | 141 | 23.629 | −13.084 | 9.502 | 1.00 | 28.21 |
| ATOM | 2763 | O | HOH | W | 142 | 31.635 | 7.713 | 14.307 | 1.00 | 30.30 |
| ATOM | 2764 | O | HOH | W | 143 | 6.811 | −2.344 | −0.868 | 1.00 | 24.47 |
| ATOM | 2765 | O | HOH | W | 144 | 7.278 | −9.981 | 30.756 | 1.00 | 27.03 |
| ATOM | 2766 | O | HOH | W | 145 | −6.981 | 7.089 | 6.281 | 1.00 | 26.94 |
| ATOM | 2767 | O | HOH | W | 146 | −8.635 | 18.558 | 29.861 | 1.00 | 27.91 |
| ATOM | 2768 | O | HOH | W | 147 | 30.525 | −1.680 | 34.334 | 1.00 | 24.09 |
| ATOM | 2769 | O | HOH | W | 148 | 2.251 | −13.867 | 2.121 | 1.00 | 25.49 |
| ATOM | 2770 | O | HOH | W | 149 | −8.063 | −9.740 | 29.191 | 1.00 | 24.34 |
| ATOM | 2771 | O | HOH | W | 150 | −1.333 | 15.279 | 37.020 | 1.00 | 28.81 |
| ATOM | 2772 | O | HOH | W | 151 | 18.429 | 15.610 | 21.408 | 1.00 | 22.50 |
| ATOM | 2773 | O | HOH | W | 152 | 13.471 | −4.264 | 12.528 | 1.00 | 19.38 |
| ATOM | 2774 | O | HOH | W | 153 | 18.621 | −17.600 | 7.222 | 1.00 | 21.52 |
| ATOM | 2775 | O | HOH | W | 154 | 19.181 | −5.396 | 13.023 | 1.00 | 33.83 |
| ATOM | 2776 | O | HOH | W | 155 | −3.698 | −11.419 | 33.622 | 1.00 | 22.32 |
| ATOM | 2777 | O | HOH | W | 156 | 24.773 | −7.687 | 26.828 | 1.00 | 25.23 |
| ATOM | 2778 | O | HOH | W | 157 | 11.338 | 6.981 | 10.450 | 1.00 | 25.98 |
| ATOM | 2779 | O | HOH | W | 158 | 7.187 | 4.097 | 9.722 | 1.00 | 25.73 |
| ATOM | 2780 | O | HOH | W | 159 | 10.571 | 18.276 | 18.940 | 1.00 | 26.56 |
| ATOM | 2781 | O | HOH | W | 160 | 29.444 | 8.138 | 18.655 | 1.00 | 25.10 |
| ATOM | 2782 | O | HOH | W | 161 | −4.733 | −16.782 | 12.321 | 1.00 | 23.51 |
| ATOM | 2783 | O | HOH | W | 162 | 24.194 | 7.218 | 40.602 | 1.00 | 28.88 |
| ATOM | 2784 | O | HOH | W | 163 | 13.925 | −7.040 | −5.961 | 1.00 | 29.58 |
| ATOM | 2785 | O | HOH | W | 164 | −8.456 | 17.425 | 10.469 | 1.00 | 25.03 |
| ATOM | 2786 | O | HOH | W | 165 | 20.283 | 7.809 | 6.109 | 1.00 | 25.58 |
| ATOM | 2787 | O | HOH | W | 166 | 11.226 | 1.360 | 14.389 | 1.00 | 28.28 |
| ATOM | 2788 | O | HOH | W | 167 | 17.600 | 11.985 | 25.766 | 1.00 | 28.21 |
| ATOM | 2789 | O | HOH | W | 168 | −1.369 | 0.364 | 6.150 | 1.00 | 26.61 |
| ATOM | 2790 | O | HOH | W | 169 | 1.250 | 6.912 | 6.112 | 1.00 | 22.96 |
| ATOM | 2791 | O | HOH | W | 170 | 13.982 | 16.820 | 19.827 | 1.00 | 26.70 |
| ATOM | 2792 | O | HOH | W | 171 | 8.401 | 6.117 | 8.285 | 1.00 | 29.37 |
| ATOM | 2793 | O | HOH | W | 172 | 1.561 | 32.980 | 20.315 | 1.00 | 25.71 |
| ATOM | 2794 | O | HOH | W | 173 | 8.687 | −4.960 | 0.724 | 1.00 | 20.63 |
| ATOM | 2795 | O | HOH | W | 174 | 16.762 | −8.584 | 32.541 | 1.00 | 22.53 |
| ATOM | 2796 | O | HOH | W | 175 | −8.928 | 23.790 | 25.122 | 1.00 | 26.68 |
| ATOM | 2797 | O | HOH | W | 176 | 30.774 | −10.692 | 12.781 | 1.00 | 29.33 |
| ATOM | 2798 | O | HOH | W | 177 | −4.491 | −0.583 | 48.435 | 1.00 | 27.59 |
| ATOM | 2799 | O | HOH | W | 178 | −10.254 | 8.887 | 25.677 | 1.00 | 27.41 |
| ATOM | 2800 | O | HOH | W | 179 | 12.487 | −14.347 | 0.366 | 1.00 | 29.02 |
| ATOM | 2801 | O | HOH | W | 180 | −2.194 | −3.193 | 5.572 | 1.00 | 37.26 |
| ATOM | 2802 | O | HOH | W | 181 | 21.240 | −14.747 | 4.723 | 1.00 | 24.12 |
| ATOM | 2803 | O | HOH | W | 182 | 25.238 | 15.872 | 21.083 | 1.00 | 33.15 |
| ATOM | 2804 | O | HOH | W | 183 | 27.650 | 8.541 | 12.497 | 1.00 | 23.15 |
| ATOM | 2805 | O | HOH | W | 184 | 35.303 | 3.837 | 26.688 | 1.00 | 23.28 |
| ATOM | 2806 | O | HOH | W | 185 | −3.730 | 14.555 | 34.107 | 1.00 | 23.34 |
| ATOM | 2807 | O | HOH | W | 186 | −8.927 | −4.660 | 10.381 | 1.00 | 28.70 |
| ATOM | 2808 | O | HOH | W | 187 | 40.695 | −1.179 | 15.166 | 1.00 | 31.35 |
| ATOM | 2809 | O | HOH | W | 188 | −0.356 | −22.615 | 31.607 | 1.00 | 35.28 |
| ATOM | 2810 | O | HOH | W | 189 | 3.463 | 34.674 | 22.382 | 1.00 | 27.20 |
| ATOM | 2811 | O | HOH | W | 190 | 8.911 | 2.196 | 11.376 | 1.00 | 19.30 |
| ATOM | 2812 | O | HOH | W | 191 | −2.598 | −24.115 | 28.861 | 1.00 | 26.04 |
| ATOM | 2813 | O | HOH | W | 192 | 8.418 | −15.510 | 34.179 | 1.00 | 38.02 |
| ATOM | 2814 | O | HOH | W | 193 | 15.703 | −17.633 | −0.060 | 1.00 | 35.88 |
| ATOM | 2815 | O | HOH | W | 194 | 32.518 | 0.997 | 3.614 | 1.00 | 34.25 |
| ATOM | 2816 | O | HOH | W | 195 | 19.062 | −16.798 | 10.198 | 1.00 | 27.69 |
| ATOM | 2817 | O | HOH | W | 196 | 13.216 | 8.976 | 10.392 | 1.00 | 27.61 |
| ATOM | 2818 | O | HOH | W | 197 | 2.647 | −0.086 | 12.019 | 1.00 | 23.91 |
| ATOM | 2819 | O | HOH | W | 198 | −1.276 | −7.561 | 35.325 | 1.00 | 25.96 |
| ATOM | 2820 | O | HOH | W | 199 | 4.267 | −22.362 | 30.170 | 1.00 | 35.68 |
| ATOM | 2821 | O | HOH | W | 200 | 21.787 | 10.595 | 30.558 | 1.00 | 32.19 |
| ATOM | 2822 | O | HOH | W | 201 | 21.638 | 20.389 | 14.210 | 1.00 | 43.09 |
| ATOM | 2823 | O | HOH | W | 202 | 5.373 | −10.297 | 38.927 | 1.00 | 36.29 |
| ATOM | 2824 | O | HOH | W | 203 | 29.035 | −9.588 | 1.139 | 1.00 | 25.61 |
| ATOM | 2825 | O | HOH | W | 204 | 0.403 | −17.629 | 11.462 | 1.00 | 25.52 |
| ATOM | 2826 | O | HOH | W | 205 | 15.896 | −2.824 | 23.429 | 1.00 | 9.81 |
| ATOM | 2827 | O | HOH | W | 206 | 13.783 | −1.283 | 21.923 | 1.00 | 12.03 |
| ATOM | 2828 | O | HOH | W | 207 | 16.854 | −0.738 | 21.328 | 1.00 | 11.76 |
| ATOM | 2829 | O | HOH | W | 208 | 14.946 | −0.465 | 19.263 | 1.00 | 10.55 |
| ATOM | 2830 | O | HOH | W | 209 | −5.566 | −9.500 | 29.835 | 1.00 | 34.12 |
| ATOM | 2831 | O | HOH | W | 210 | 11.435 | 14.251 | 33.693 | 1.00 | 48.96 |
| ATOM | 2832 | O | HOH | W | 211 | 3.944 | 31.281 | 18.816 | 1.00 | 43.83 |
| ATOM | 2833 | O | HOH | W | 212 | 21.511 | 8.201 | 40.401 | 1.00 | 42.14 |
| ATOM | 2834 | O | HOH | W | 213 | 44.825 | 1.178 | 19.793 | 1.00 | 52.54 |
| ATOM | 2835 | O | HOH | W | 214 | 9.397 | 20.244 | 23.958 | 1.00 | 30.91 |
| ATOM | 2836 | O | HOH | W | 215 | 18.636 | −1.182 | 15.865 | 1.00 | 23.82 |
| ATOM | 2837 | O | HOH | W | 216 | 18.955 | −2.693 | 17.957 | 1.00 | 28.82 |
| ATOM | 2838 | O | HOH | W | 217 | 26.248 | −9.541 | 27.965 | 1.00 | 25.10 |
| ATOM | 2839 | O | HOH | W | 218 | 24.703 | −9.867 | 30.146 | 1.00 | 25.52 |
| ATOM | 2840 | O | HOH | W | 219 | 22.600 | −9.260 | 25.832 | 1.00 | 26.71 |
| ATOM | 2841 | O | HOH | W | 220 | 1.825 | 10.054 | 39.457 | 1.00 | 25.12 |
| ATOM | 2842 | O | HOH | W | 221 | 3.585 | 4.590 | 45.058 | 1.00 | 28.59 |
| ATOM | 2843 | O | HOH | W | 222 | 3.777 | −6.424 | 30.447 | 1.00 | 24.42 |
| TER | 2844 | | HOH | W | 222 | | | | | |
| ATOM | 2845 | ZN | ZN | B | 864 | 13.210 | 0.069 | 20.406 | 1.00 | 15.15 |
| ATOM | 2846 | MG | MG | B | 865 | 15.487 | −0.725 | 23.006 | 1.00 | 8.84 |
| TER | 2847 | | MG | B | 865 | | | | | |
| END | | | | | | | | | | |

### REFERENCES

Abrahams, J. P. & Leslie, A. (1996) Acta Crystallogr. D 52, 30-42.

Aravind, L. & Ponting, C. P. (1997) TIBS. 22, 458-459.

Brünger, A. et al. (1998), Acta Crystallogr. D 54, 905-921.

Butcher, R. W. & Sutherland, E. W. (1962) J. biol. Chem. 237, 1244-1250.

Charbonneau, H. (1990) in Cyclic Nucleotide Phosphodiesterases: Structure, Regulation and Drug Action (Beavo, J., and Houslay, M. D., eds) pp. 267-296, John Wiley & Sons, Inc., New York.

Charbonneau, H. et al., (1986) Proc. Natl. Acad. Sci. U. S. A., 83, 9308-9312.

Collaborative Computational Project Number 4 (1994) Acta Crystallogr. D 50, 760-763.

Corbin, J. D. & Francis, S. H. (1999) J. Biol. Chem. 274, 13729-13732.

de La Fortelle, E. & Bricogne, G. (1997) Methods Enzymol. 276, 472-494.

Francis, S. H. et al. (1994) J. Biol. Chem. 269, 22477-22480.

Goldberg, N. D. et al. (1980) J. Biol. Chem. 255, 10344-10347.

Hendrickson, W. A. et al. (1989) Basic Life Sci. 51 (Synchrotron Radiat. Struct. Biol.), 317-324.

Jiang, H. et al. (1992) J. Biol. Chem. 267, 1015-1019.

Laskowski, R. A. et al. (1993) J. Appl. Crystallogr. 26, 283-291.

Lohman, S. M. et al. (1997) Trends in Biochem. Sci. 22, 307-312.

Martinez, S. et al. (2001) Poster presented at the American Crystallography Association annual meeting, Los Angeles, USA.

Matthews, B. W. (1968) J. Mol. Biol. 33, 491-497.

Nicholls, A. et al. (1993) J. Biophys, A166.

Otwinowski, Z. & Minor, W. (1997) Methods Enzymol. 276, 307-326.

Quanta98, 1998, version 98.1111; Molecular Simulations Inc., San Diego, Calif. 92121-3752.

Rall, T. W. & Sutherland, E. W. (1958) J. Biol. Chem. 232, 1065-1076.

Soderling, S. H. & Beavo, J. A., (2000) Curr. Opin. Cell Biol. 12, 174-179.

Smith, G. D. et al. (1998) Acta Crystallographica Section D-Biological Crystallography. 54 (Pt 5):799-804

Terwilliger, T. C. & Berendzen, J. (1997) Acta Crystallogr. D 53, 571-579 Crystallogr. D 54, 799-804.

Thomas et al. (1990) J. Biol. Chem. 265, 14971-14978.

Xu, R. X. et al. (2000) Science 288, 1822-1825.

J.Navaza, (1994) Acta Cryst. A50, 157-163.

### Abbreviations

| | |
|---|---|
| cAMP | cyclic adenosine monophosphate |
| cGMP | cyclic guanosine monophosphate |
| PDE | phosphodiesterase |
| PGK | protein kinase G |
| MAD | multi-wavelength anomalous dispersion |
| PCR | polymerase chain reaction |
| 2YT | 16 g tryptone, 10 g yeast extract, 5 g NaCl per litre |
| | solution |
| Tris | tris[hydroxymethyl]amino-methane |
| E-64 | epoxysuccinyl-1-leucylamido-(4-guanidino) butane |
| DTT | DL-dithiothreitol |
| β-ME | β-mercaptoethanol |
| IPTG | β-D-isopropyl-thiogalactopyranoside |
| EDTA | ethylenediamine tetraacetic acid |
| Bis-Tris | bis[2-hydroxyethyl]imino-tris[hydroxymethyl]methane |
| PEG | polyethylene glycol |
| PEG2KMME | polyethylene glycol 2000 monomethyl ether |
| rmsd | root mean square deviation |
| rpm | revolutions per minute |
| Moi | multiplicity of infection |
| Sildenafil | 5-[2-ethoxy-5-(4-methyl-1-piperazinylsulphonyl)phenyl]- |
| | 1-methyl-3-n-propyl-1,6-dihydro-7H-pyrazolo[4,3-d] |
| | pyrimidin-7-one, which is also known as 1-[[3-(6,7- |
| | dihydro-1-methyl-7-oxo-3-propyl-1H-pyrazolo[4,3-d] |
| | pyrimidin-5-yl)-4-ethoxyphenyl]sulphonyl]-4- |
| | methylpiperazine (see EP-A-0463756) |
| UK-092,480 | see “Sildenafil” |
| UK-088,800 | 5-(2-ethoxyphenyl)-1-methyl-3-propyl-1,6-dihydro-7H- |
| | pyrazolo[4,3-d]pyrimidin-7-one. |

### List of Sequences

| | |
|---|---|
| Wild-type PDE5 “loop region” | |
| HRGVNNSYIQRSEHPLAQLYCHSIME | SEQ ID NO:1 |
| Wild-type PDE5 catalytic domain | |
| EEETRELQSLAAAVVPSAQTLKITDFSFSDFELSDLETALCTIRMFTDLNLVQNF | SEQ ID NO:2 |
| QMKHEVLCRWILSVKKNYRKNVAYHNWRHAFNTAQCMFAALKAGKIQNKL | |
| TDLEILALLIAALSHDLDHRGVNNSYIQRSEHPLAQLYCHSIMEHHHFDQCLMI | |
| LNSPGNQWSGLSIEEYKTTLKIIKQAWATDLALYTKRRGEFFEURKNQFNLEDP | |
| HQKELFLAMLMTACDLSAITKPWPIQQRIAELVATEFFDQGDRERKELNIEPTD | |
| LMNREKKNKIPSMQVGFTDAICLQLYEALTHVSEDCFPLLDGCRKNRQKWQA | |
| LAEQQEKMLINGESGQAKRN | |
| Full length wild-type PDE5 sequence | |
| ERAGPSFGQQRQQQQPQQQKQQQRDQDSVEAWLDDHWDFTFSYFVRKATRE | SEQ ID NO:3 |
| MVNAWFAERVHTIPVCKEGIRGHTESCSCPLQQSPRADNSVPGTPTRKISASEF | |
| DRPLRPIVVKDSEGTVSFLSDSEKKEQMPLTPPRFDHDEGDQCSRLLELVKDIS | |
| SHLDVTALCHKIELHIHGLISADRYSLFLVCEDSSNDKFLISRLFDVAEGSTLEEV | |
| SNNCTRLEWNIKIGIVGHVAALGEPLNTKDAYEDPRFNAEVDQITGYKTQSILCM | |
| PTKNITREEVVGVAQAINKKSGNGGTFTEKDEKDFAAYLAFCGIVLHNAQLYET | |
| SLLENKRNQVLLDLASLWEEQQSLEVILKKIAATHSFMQVQKCTIFIVDEDCSD | |
| SFSSVFHMECEELEKSSDTLTREHDANKINYMYAQYVKNTMEPLNIPDVSKDK | |
| RFPWTTENTGNVNQQCIRSLLCTPIKNGKKNKVIGVCQLVNKMEENTGKVKPF | |
| NRNDEQFLEAFVIIFCGLGIQNTQMYEAVERAMAKQMVTLEVLSYHASAAEEE | |
| TRELQSLAAAVVPSAQTLKITDFSFSDFELSDLETALCTIRMFTDLNLVQNFQM | |
| KHEVLCRWILSVKKINYRKNVAYHNWRHAFNTAQCMFAALKAGKIQNKLTDL | |
| EILALLIAALSHDLDHRGVNNSYIQRSEHPLAQLYCHSIMEHHHFDQCLMILNS | |
| PGNQILSGLSIEEYKTTLKIIKQAILATDLALYIKRRGEFFELIRKNQFNLEDPHQ | |
| KELFLAMLMTACDLSAITKPWPIQQRIAELVATEFFDQGDRERKELNWPTDLM | |
| NREKKNKIPSMQVGFIDAICLQLYEALTHVSEDCFPLLDGCRKNRQKWQALAE | |
| QQEKMLTNGESGQAKRN | |
| Wild-type PDE4 “loop region” | |
| HPGVSNQFLINTNSELALMYNDESVLE | SEQ ID NO:4 |
| Loop-swapped PDE5 catalytic domain =PDE5 * | |
| EEETRELQSLAAAVVPSAQTLKITDFSFSDFELSDLETALCTTRMFTDLNLVQNF | SEQ ID NO:5 |
| QMKHEVLCRWILSVKXNYRKNVAYHNWRHAFNTAQCMFAALKAGKIQNKL | |
| TDLEILALLIAALSHDLDHPGVSNQFLINTNSELALMYNDESVLEHHHFDQCLM | |
| ILNSPGNQILSGLSIEEYKTTLKIIKQAILATDLALYIKRRGEFFELIRKNQFNLED | |
| PHQKELFLAMLMTACDLSAITKPWPIQQRIAELVATEFFDQGDRERKELNIEPT | |
| DLMNREKKNKJPSMQVGFIDATCLQLYEALTHVSEDCFPLLDGCRKNRQKWQ | |
| ALAE | |
| Full length PDES sequence comprising PDE5 * | |
| ERAGPSFGQQRQQQQPQQQKQQQRDQDSVEAWLDDHWDFTFSYFVRKATRE | SEQ ID NO:6 |
| MVNAWFAERVHTIPVCKEGIRGHTESCSCPLQQSPRADNSVPGTPTRKISASEF | |
| DRPLRPIVVKDSEGTVSFLSDSEKKEQMPLTPPRFDHDEGDQCSRLLELVKDIS | |
| SHLDVTALCHWLHIHGLISADRYSLFLVCEDSSNPKFLISRLFDVAEGSTLEEV | |
| SNNCIIRLEWNKGIVGHVAALGEPLNIKDAYEDPRFNAEVDQITGYKTQSILCM | |
| PIKNHREEVVGVAQAINKKSGNGGTFTEKDEKDFAAYLAFCGIVLHNAQLYET | |
| SLLENKRNQVLLDLASLWEEQQSLEVILKKIAATIISFMQVQKCTWWDEDCSD | |
| SFSSVFHMECEELEKSSDTLTREHDANKINYMYAQYVKNTMEPLNIPDVSKDK | |
| RFPWTTENTGNVNQQCIRSLLCTPIKNGKKNKVIGVCQLVNKMEENTGKVKPF | |
| NRNDEQFLEAFVIFCGLGIQNTQMYEAVERAMAKQMVTLEVLSYHASAA | |
| EEETRELQSLAAAVVPSAQTLKITDFSFSDFELSDLETALCTIRMFTDLNLVQNF | |
| QMKHEVLCRWILSVKKNYRKNVAYHNWRHAFNTAQCMFAALKAGKIQNKL | |
| TDLEILALLIAALSHDLDHPGVSNQFLINTNSELALMYNDESVLEHHHFDQCLM | |
| ILNSPGNQILSGLSIEEYKTTLKIIKQAILATDLALYTKRRGEFFELIRKNQFNLED | |
| PHQKELFLAMLMTACDLSAITKPWPIQQRIAELVATEFFDQGDRERKELNIEPT | |
| DLMNREKKNXIPSMQVGFIIDAICLQLYEALTHVSEDCFPLLDGCRRNRQKWQ | |
| ALAE | |
| CGTGAATTCATGGAGGAAGAAACAAGAGAGCTAC | SEQ ID NO:7 |
| CGTTCTAGACTATCAGTTCCGCTTGGCCTGGCCGCTTTCCCC | SEQ ID NO:8 |
| CGTGAATTCATGCATCATCATCATCATCATCTTCTGGTTCCGCGTGGATCTG | SEQ ID NO:9 |
| CGCCCGAGGAAGAAACAAGAGAGCTAC | |
| CAAAGAAAGTTCTGAATTTGTGTTGATGAGAAACTGATTGGAGACTCCAG | SEQ ID NO:10 |
| GATGATCCAAATCGTGGCTTAG | |
| ATCAACACAAATTCAGAACTTGCTTTGATGTATAATGATGAATCTGTGTTG | SEQ ID NO:11 |
| GAACACCATCATTTTGACCAG | |
| CGTTCTAGACTATCATTCTGCAAGGGCCTGCCATTTCTG | SEQ ID NO:12 |
| CGTCATATGGAGGAAGAAACAAGAGAGCTAC | SEQ ID NO:13 |
| CGTCTCGAGCTATCATTCTGCAAGGGCCTGCCATTTCTG | SEQ ID NO:14 |

1. A crystal of phosphodiesterase 5 (PDE5).

2. The crystal of PDE5 according to claim 1, wherein the crystal is soakable.

3. The crystal of PDE5 according to any one of claims 1 or 2, wherein said PDE5 is from a mammal.

4. The crystal of PDE5 according to any one of claims 1 to 3, wherein said PDE5 is from a human.

5. The crystal of PDE5 according to any one of claims 1 to 4, wherein said PDE5 is an isoform selected from the group including PDE5A1, PDE5A2, PDE5A3 and PDE5A4.

6. The crystal of PDE5 according to any one of claims 1 to 5, wherein said PDE5 comprises SEQ ID NO: 1 or a homologue, fragment, variant, analogue or derivative thereof.

7. The crystal of PDE5 according to any one of claims 1 to 6, wherein said PDE5 comprises SEQ ID NO: 4 or a homologue, fragment, variant, analogue or derivative thereof.

8. The crystal of PDE5 according to any one of claims 1 to 7, wherein said PDE5 comprises SEQ ID NO: 5 or a homologue, fragment, variant, analogue or derivative thereof.

9. The crystal of PDE5 according to any one of claims 1 to 8, wherein said PDE5 comprises SEQ ID NO: 6 or a homologue, fragment, variant, analogue or derivative thereof.

10. The crystal of PDE5 according to any one of the claims 1 to 9 which is grown using polyethylene glycol as a precipitant.

11. The crystal of PDE5 according to any one of claims 1 to 10 which is grown in a buffer in the pH range of 6.5 to 8.0.

12. The crystal of PDE5 according to any one of the claims 1 to 11 which is grown in the presence of an alcohol.

13. The crystal of PDE5 according to any one of the claims 1 to 12 which is grown in a solution containing HEPES buffer, polyethylene glycol 4000 and iso-propanol.

14. The crystal of PDE5 as defined in any one of claims 1 to 13, which has one or more of the following characteristics:
(g) a space group C2;
(h) unit cell dimensions a˜56 Å±1%, b˜77 Å±1%, c˜81 Å±1%, α=γ=90°, β=103°±1%;
(i) 1 molecule per asymmetric unit;
(J) comprises a PDE5 of a molecular weight of approximately 4 OkDa±2 kDa;
(k) a calculated solvent content of approximately 47±5%; and
(1) a monoclinic crystal system.

15. The crystal of PDE5 as claimed in any one of claims 1 to 14, wherein PDE5 has an active site within the third sub-domain of the protein which is bounded by Helices 15 (H15 813-824) and 14 (H14 772-797), the C-terminus of Helix 13 (H13 749-25 765), and the C-terminus of Helix 11 (H11 706-721) along with the loop region between Helices 11 and 12a (H12a 725-731) as shown in FIG. 2.

16. The crystal of PDE5 as claimed in claim 15, wherein the active site is capable of accommodating a pyrazolo-pyrimidinone.

17. The crystal of PDE5 as claimed in any one of the claims 1 to 16, wherein PDE5 has an active site within the third sub-domain of the protein and comprises Leu 765, Ala 767 and Ile 768 and one or more of Phe 820, Val 782, Phe 786, Tyr 612, Leu 804, Ala 779, Ala 783, Ile 813, Met 816 and Gln 817.

18. The crystal of PDE5 as claimed in claim 17, wherein the active site is capable of accommodating a ligand or inhibitor.

19. The crystal of PDE5 as claimed in any one of the claims 1 to 18, wherein said PDE5 has a three-dimensional structure characterised by the atomic co-ordinates set out in Table 4 or a derivative set as expressed in any reference frame.

20. A heavy atom derivative of the crystal of PDE5 as claimed in any one of the claims 1 to 19.

21. The crystal of PDE5 as claimed in any one of claims 1 to 20, wherein a PDE5 ligand has been soaked in.

22. The crystal of PDE5 as claimed in claim 22, wherein said PDE5 ligand is a PDE5 inhibitor.

23. The crystal of PDE5 as claimed in claim 22, wherein said PDE5 inhibitor is a pyrazolo-pyrimidinone.

24. The crystal of PDE5 as claimed in any one of claims 21 to 23, wherein said PDE5 has a three-dimensional structure characterised by the atomic co-ordinates set out in Table 5 or a derivative set as expressed in any reference frame.

25. Use of the atomic co-ordinates determined from the crystal of PDE5 according to any one of claims 1 to 24 for deriving a three-dimensional structure of a PDE5 or a mutant, derivative, variant, analogue, homologue, sub-domain or fragment thereof.

26. Use according to claim 25, wherein the PDE5 sub-domain is the catalytic domain.

27. Use of the three-dimensional structure of PDE5 as derivable according to one of claim 25 or claim 26 to computationally or otherwise evaluate the binding interactions of a chemical compound with an active site on PDE5.

28. Use of the three-dimensional structure of PDE5 as derivable according to claim 27 to design a compound capable of associating with PDE5.

29. Use according to claim 27 or claim 28, wherein the compound is a PDE5 ligand.

30. Use according to claim 29, wherein said compound is a PDE5 inhibitor.

31. Use according to claim 30, wherein said PDE5 inhibitor is a pyrazolo-pyrimidinone.

32. A method of selecting a compound capable of associating with PDE5 from a group of potential PDE5 ligand compounds comprising the following steps:
i. soaking the crystal of PDE5 according to any one of claims 1 to 19 in a solution containing a potential PDE5 ligand compound;
ii. determining the three-dimensional structure from the soaked crystal; and
iii. assessing whether the compound is bound to PDE5.

33. A compound selected by the use according to claim 32.

34. A compound designed by the use according to any one of claims 28 to 31 or identified by the method of claim 32.

35. The compound according to claim 34, which is a PDE5 inhibitor.

36. A method of selecting a PDE5 ligand from a group of potential PDE5 ligands, comprising the following steps:
(a) computationally creating a three-dimensional representation of the structure of 30 PDE5 as derivable according to any one of claim 25 or claim 26, and a three-dimensional representation of the structure of the potential PDE5 ligand;
(b) co-displaying the three-dimensional representation of the potential PDE5 ligand with the three-dimensional representation of the PDE5 structure; and
(c) assessing whether the three-dimensional representation of the potential PDE5 ligand fits the three-dimensional representation of an active site of the PDE5 structure.

37. The method according to claim 36, further comprising the following steps:
(d) incorporating the potential PDE5 ligand in a biological PDE5 activity assay; and
(e) determining whether the potential PDE5 ligand modulates PDE5 activity in said assay.

38. The method according to any one of claim 36 or claim 37 wherein said potential PDE5 ligand is a potential PDE5 inhibitor compound and said potential PDE5 inhibitor compound inhibits PDE5 activity.

39. A PDE5 ligand selected by the method of any one of claims 36 to 38.

40. Use of a PDE5 ligand according to claim 39 as a pharmaceutical.

41. A pharmaceutical composition comprising one or more PDE5 ligands according to claim 39 and one or more pharmaceutically acceptable excipients.

42. Use of a PDE5 ligand according to claim 39 in the manufacture of a medicament for the prophylaxis or treatment of a condition, disease, disorder or dysfunction where the inhibition of PDE5 is prophylactically or therapeutically beneficial.

43. Use according to claim 42, wherein said disorder is a mammalian sexual disorder.

44. Use of the atomic co-ordinates determined from the crystal of PDE5 according to any one of claims 1 to 24, to solve the crystal structure of a mutant, derivative, fragment, variant, analogue, homologue or complex of PDE5.

45. Use of the atomic co-ordinates determined from the crystal of PDE5 according to any one of claims 1 to 24, to produce a model of the three-dimensional structure of PDE5-related proteins.

46. Use of the three-dimensional structure of PDE5 as derivable according to any one of claim 25 or claim 26 to design site-directed mutants that mimic other PDE5 isoforms or variants thereof.

47. A method of soaking a chemical compound into a crystal according to any one of claims 1 to 20 comprising the following steps:
a) incubating the crystal in an aqueous stabilising solution comprising buffer lo and polyethylene glycol;
b) combining the chemical compound with the stabilising solution; and
c) optionally adding a cryo-protectant to the stabilising solution.
